(19) United States
(12) Patent Application Publication
(10) Pub. No.: US 2025/0214970 A1
(43) Pub. Date: Jul. 3, 2025

(54) PYRIDINYLACETAMIDE DERIVATIVES AS SODIUM CHANNEL ACTIVATORS

(71) Applicant: Xenon Pharmaceuticals Inc., Burnaby (CA)

(72) Inventors: Verner Alexander LOFSTRAND, Burnaby (CA); Jung Yun KIM, Burnaby (CA); Helen CLEMENT, Burnaby (CA); Kristen Nicole BURFORD, Burnaby (CA); Paul CHARIFSON, Burnaby (CA); Shawn JOHNSTONE, Burnaby (CA); Juliette SABBATANI, Burnaby (CA); Jan Felix SCHOLTES, Burnaby (CA); Wei ZHANG, Burnaby (CA); Shaoyi SUN, Coquitlam (CA); Michael CLARK, Burnaby (CA); Steve WESOLOWSKI, Burnaby (CA); Ravi MUNUGANTI, Burnaby (CA); Ramkumar RAJAMANI, Burnaby (CA)

(21) Appl. No.: 18/937,863

(22) Filed: Nov. 5, 2024

Related U.S. Application Data
(63) Continuation of application No. 17/951,644, filed on Sep. 23, 2022, now abandoned.
(60) Provisional application No. 63/248,334, filed on Sep. 24, 2021.

(51) Int. Cl. C07D 401/14; C07D 213/74; C07D 213/82; C07D 401/04; C07D 403/04; C07D 403/14; C07D 405/14; C07D 413/14; C07D 417/14; C07D 471/04; C07D 471/08; C07D 471/10; C07D 471/14; C07D 487/04; C07D 487/10; C07D 491/107; C07D 498/08; C07F 7/08

(52) U.S. Cl. CPC C07D 401/14; C07D 213/74; C07D 213/82; C07D 401/04; C07D 403/04; C07D 403/14; C07D 405/14; C07D 413/14; C07D 417/14; C07D 471/04; C07D 471/08; C07D 471/10; C07D 471/14; C07D 487/04; C07D 487/10; C07D 491/107; C07D 498/08; C07F 7/0812

(57) ABSTRACT

The present disclosure is directed to compounds of formula (I):

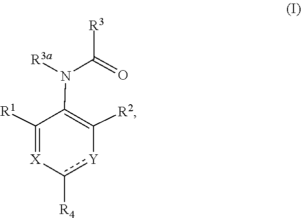

wherein R¹, R², R³, R^{3a}, R⁴, Y, and X are as described herein, as stereoisomers, enantiomers, or tautomers thereof or mixtures thereof; or pharmaceutically acceptable salts, solvates, or prodrugs thereof, and pharmaceutical compositions comprising the compounds of formula (I), as described herein, which are useful as voltage-gated sodium channel modulators and are therefore are useful in treating seizure disorders such as epilepsy.

### BACKGROUND

### Technical Field

This disclosure is directed to pyridinylacetamide derivatives, as stereoisomers, enantiomers, or tautomers thereof or mixtures thereof; or pharmaceutically acceptable salts, solvates, or prodrugs thereof, and pharmaceutical compositions comprising the pyridinylacetamide derivatives, which are useful as voltage-gated sodium channel activators and are therefore are useful in treating seizure disorders such as epilepsy.

### Description of the Related Art

Epilepsy is a common seizure disorder, with a worldwide estimated prevalence of 0.7% of the population (50 million people) (see Hirtz, D. et al., Neurology. (2007), 68:326-337). It is characterized by abnormal electrical activities in the brain leading to seizures. For epidemiological purposes, the definition requires more than one unprovoked seizure of any type.

Patients with epilepsy have an increased mortality risk compared with the general population due primarily to the etiology of the disease. However, in patients with uncontrolled epilepsy, the greatest seizure-related risk of mortality is due to sudden unexpected death in epilepsy (SUDEP) (see, Hitiris, N. et al., Epilepsy and Behavior (2007), 10:363-376. Patients who participate in clinical trials of investigational antiepileptic drugs (AEDs) generally have had epilepsy for more than 10 years and have failed multiple AED therapies.

The pathophysiology of most forms of epilepsy remains poorly understood, but it is known that epileptic seizures arise from an excessively synchronous and sustained firing of a group of neurons. Persistent increase in neuronal excitability is common to all epileptic syndromes. The therapeutic strategy in treating epilepsy involves reducing neuronal excitability through various mechanistic pathways. Over the past two decades, several new AEDs were developed and marketed to expand the therapeutic spectrum by targeting different mechanisms of action and to improve the risk/benefit profile. Currently available AEDs are considered to act by inhibition of synaptic vesicle glycoprotein, potentiation of the inhibitory GABAergic neurotransmission, reduction of glutamate-mediated excitatory neurotransmission, or inhibition of voltage-gated sodium or calcium channels. Despite this, up to 30% of patients remain refractory to conventional treatment and continue to have uncontrolled seizures (see Brown, D. A. et al., Nature (1980), 283:673-676, and Elger, C. E. et al., Epilepsy Behav. (2008), 12:501-539. The quality of life in refractory patients is poor; they cannot drive a car, and they have difficulty working or living independently. Additionally, many patients have behavioral, neurological, and/or intellectual disturbances as sequelae of their seizure disorder. Current agents have minimal to no effects on neuronal sodium-gated channels, in spite of the fact that these channels have a major role in the control of neuronal excitability. Medicines with novel mechanisms of action, or medicines that improve on the already marketed AEDs are therefore needed to address the significant unmet clinical need for seizure control in patients with treatment-resistant epilepsy.

Nav1.1 is a voltage-gated sodium channel (Nav), comprising one pore-forming α-subunit encoded by SCN1A and two associated β-subunits encoded by SCN1B-SCN4B. Nav1.1 as well as its subfamilies (Navl.2, Nav1.3 and Nav1.6), is predominantly expressed in the central nervous system (CNS) (Catterall, W. A., J Physiol (2012), Vol. 590, pp. 2577-2589, and Catterall, W. A., Neurochem Res (2017), Vol. 42, pp. 2495-2504). Nav1.1 is largely expressed in parvalbuminpositive fast spiking interneurons (FSINs) and is involved in membrane depolarization and action potential (AP) firing (Ogiwara, I. et al., J Neurosci (2007), Vol. 27, pp. 5903-5914). Therefore, loss of function of the Nav1.1 channels could lead to disinhibition of excitatory pyramidal neurons causing various diseases of the CNS (Han, S. et al., Nature (2012), Vol. 489, pp. 385-390, Oakley, J. C. et a/. Epilepsia (2011), Vol. 52(Suppl. 2), pp. 59-61, and Verret, L. et al., Cell (2012), Vol. 149, pp. 708-721). Dravet syndrome is a rare genetic epileptic encephalopathy, where more than 70% of patients have de novo heterozygous mutations of the SCN1A gene (Catterall, W. A., Ann Rev Pharmacol Toxicol (2014), Vol. 54, pp. 317-338). In these mutations, a loss of function of the Navl.1 channels has been reported (Mantegazza, M. et al., Proc Nat/Acad Sci USA (2005), Vol. 102, pp. 18177-18182). The genetic link between Dravet syndrome patients and Nav1.1 channels suggest that a brain penetrant Nav1.1 activator could possess significant therapeutic potential for treating Dravet syndrome (Jensen, H. S. et al., Trends Pharmacol Sci (2014), Vol. 35, pp. 113-118, and Richards, K. L. et al., Proc Nat/Acad Sci USA (2018), Vol. 115, pp. E8077-E8085). However, potent and selective Nav1.1 activators have not been reported to date. Recently, a few Nav1.1 activators have been reported by Lundbeck: a 2-methylbenzamide derivative (Crestey, F. et al., ACS Chem Neurosci (2015), Vol. 6, pp. 1302-1308), AA43279 (Frederiksen, K. et al., Eur J Neurosci (2017), Vol. 46, pp. 1887-1896) and Lu AE98134 (von Schoubyea, N. L. et a/., Neurosci Lett (2018), Vol. 662, pp. 29-35). The most recently developed activator, Lu AE98134, increases the total area under the curve for the duration of the depolarizing pulse from 1 μM in Nav1.1-expressing HEK cells, while issues of low selectivity against Navl.5 and moderate selectivity against Nav1.2 were observed. Biologically, Nav1.5 is a major cardiac sodium channel (Vincent, G. M., Annu Rev Med (1998), Vol. 49, pp. 263-274) and Nav1.2 is dominantly expressed in excitatory neurons (Gong, B. et al., J Comp Neurol (1999), Vol. 412, pp. 342-352, and Hu, W. et al., Nat Neurosci (2009), Vol. 12, pp. 996-1002). Therefore, high selectivity against Nav1.5 and Nav1.2 is preferable for drug candidates. On the other hand, the electrophysiology data regarding Lu AE98134 reveals promising potency as a Navl.1 activator for increasing the excitability of FSINs. The discovery of a 4-phenyl-2-(pyrrolidinyl)nicotinamide derivative as a highly potent Nav1.1 activator with improved selectivity against Navl.2 and Navl.5 compared with previously reported Nav1.1 activators was recently published (Miyazaki, T. et al., Bioorg Med Chem Lett (2019), Vo. 29, No. 6, pp. 815-820).

While significant advances have been made in this field, there remains a substantial need for compounds which are voltage-gated sodium channel activators, thereby being useful in treating seizure disorders, preferably epilepsy, in a mammal, preferably a human.

### BRIEF SUMMARY

The present disclosure is directed to pyridinylacetamide derivatives, as stereoisomers, enantiomers, or tautomers thereof or mixtures thereof; or pharmaceutically acceptable salts, solvates, or prodrugs thereof, and pharmaceutical compositions comprising the pyridinylacetamide derivatives, which are useful as voltage-gated sodium channel activators, particularly Nav1.1 activators, and are therefore are useful in treating seizure disorders such as epilepsy and Dravet syndrome.

Accordingly, in some embodiments, the present disclosure is directed to a compound compound of formula (I):

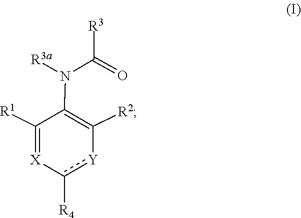

wherein:
[symbol] represents a double or single bond such that all valences are satisfied;
Y is N or NR^{4a};
X is C(R⁷) or N;
R¹ is selected from:

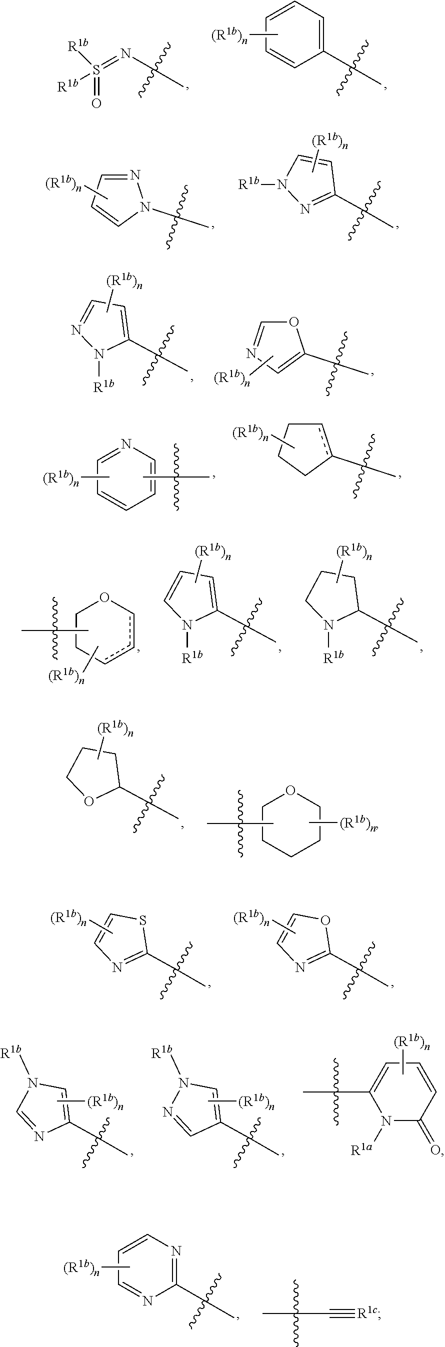

wherein:
each occurrence of [symbol] independently represents a double or single bond such that all valences are satisfied;
n is O 0, 1, 2, 3,4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
R¹⁰ is N or —Si(CH₃)₃;

R² is selected from:

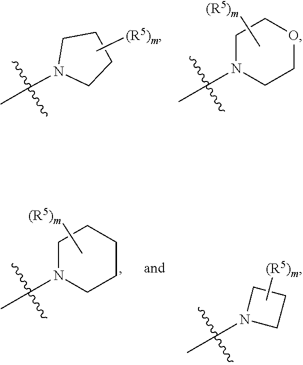

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain;

R³ is alkyl, —R⁸—N(R⁹)₂, —R⁸—OR⁹, or

R³ is selected from:

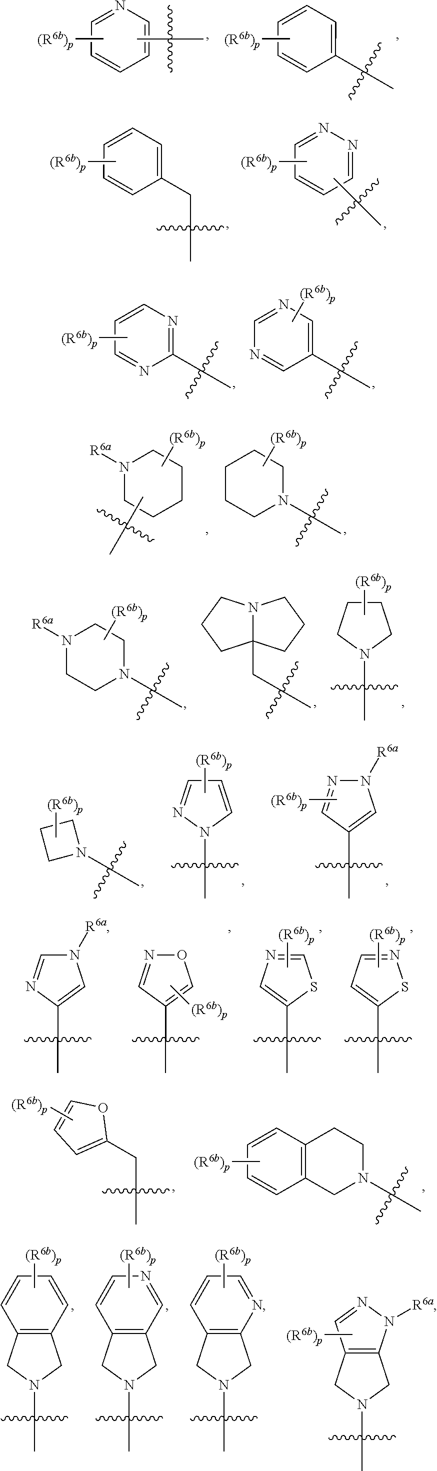

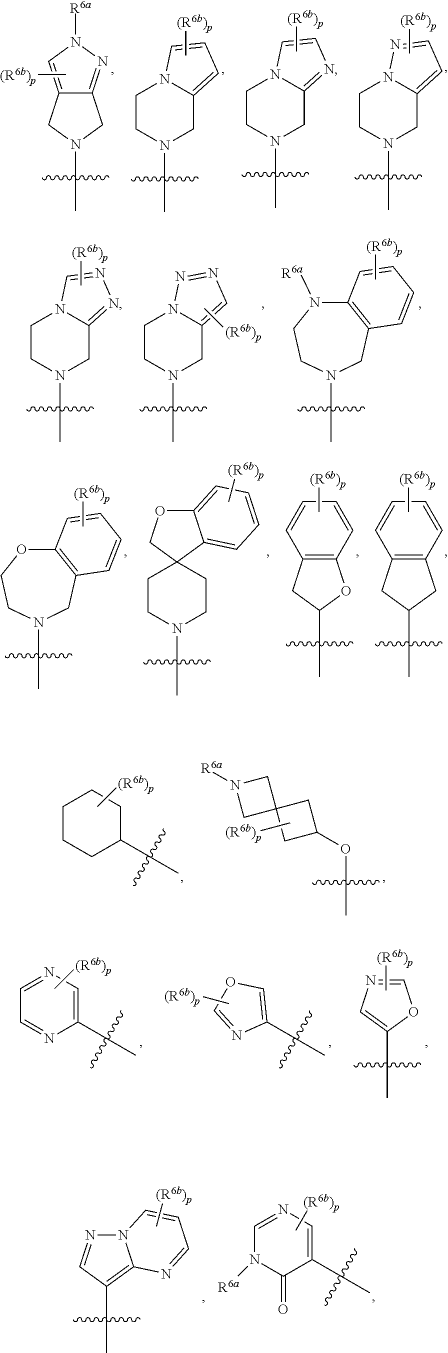

wherein:
p is 0, 1, 2, 3, 4, or 5; R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain;
or an occurrence of R^{6b} and an occurrence of R^{1b} join to form an optionally substituted alkylene chain;
R^{3a} is hydrogen or alkyl;
R⁴ is hydrogen, alkyl, —R⁸—OR⁹, halo, haloalkyl, or cyano; or R⁴ together with the carbon to which it is attached, joins with R^{4a} together with the nitrogen to which it is attached to form an optionally substituted 5-membered N-heteroaryl;
R⁷ is hydrogen, alkyl, halo, or —R⁸—OR⁹;
each R⁸ is independently a direct bond or an optionally substituted alkylene chain;
each R⁹ is independently hydrogen, alkyl, haloalkyl, carboxyalkyl, optionally substituted cycloalkyl, optionally substituted cycloalkylalkyl, or optionally substituted aryl; and
or two R⁹'s, together with the nitrogen to which they are both attached, form an optionally substituted heterocyclyl;
provided that:
when X is N, R³ is selected from:

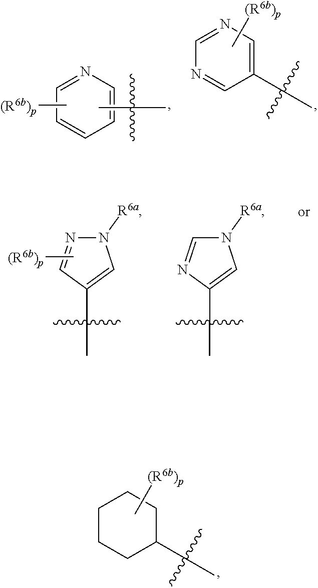

as a stereoisomer, enantiomer, or tautomer thereof or a mixture thereof; or a pharmaceutically acceptable salt, solvate, or prodrug thereof.

In some embodiments, the disclosure is directed to compounds of formula (II):

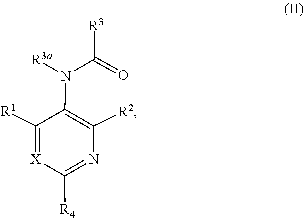

wherein:
X is C(R⁷) or N;
R¹ is selected from:

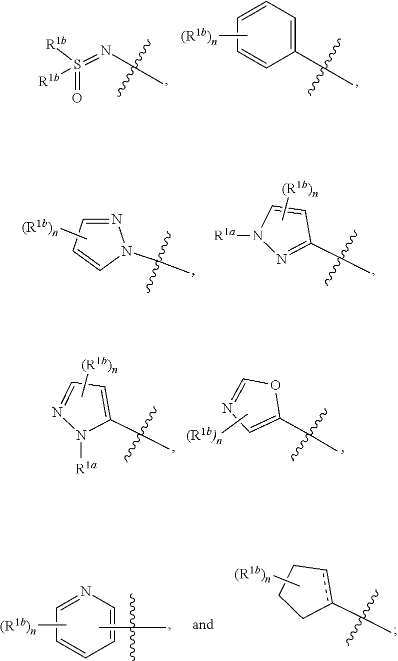

wherein:
[symbol] represents a double or single bond;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl;
R² is selected from:

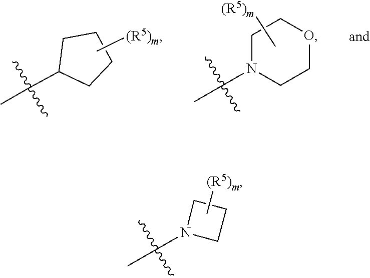

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted cycloalkyl; and
or two R⁵'s, together with the carbons to which they are attached, form an optionally substituted alkylene chain;
R³ is alkyl, —R⁸—N(R⁹)₂, —R⁸—OR⁹, or
R³ is selected from:

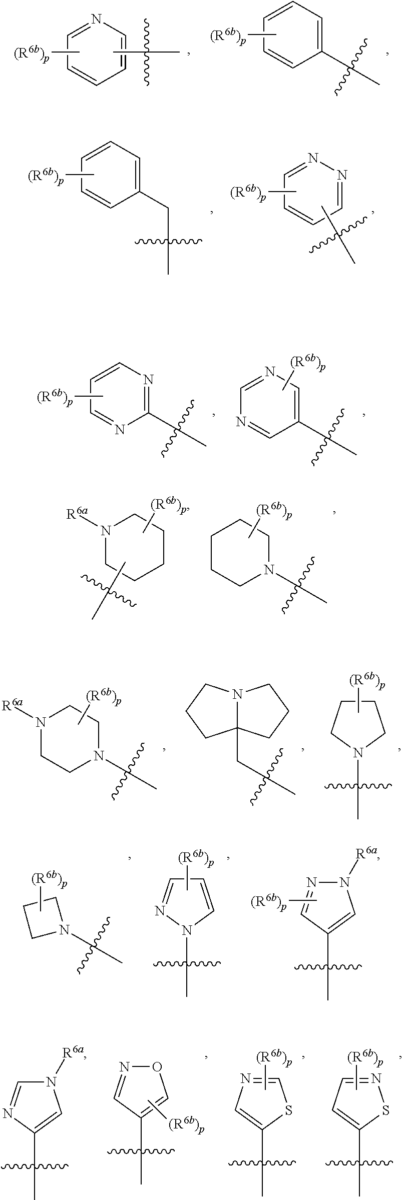

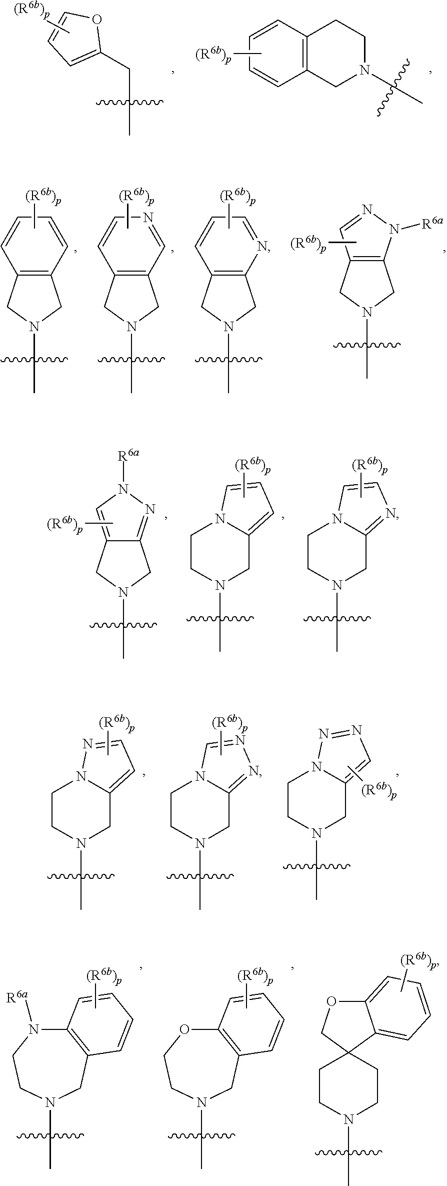

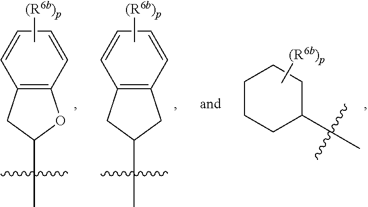

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, —R⁸—C(═O)N(R⁹)₂,optionally substituted aryl, optionally substituted heterocyclyl, or optionally substituted heteroaryl;
or two R^{6b}'s, together with the carbon to which they are both attached, form an optionally substituted N-heterocyclyl;
or two R^{8b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain;
R^{3a} is hydrogen or alkyl;
R⁴ is hydrogen, alkyl, —R⁸—OR⁹, halo, haloalkyl, or cyano,
R⁷ is hydrogen, alkyl, halo, or —R⁸—OR⁹;
each R⁸ is independently a direct bond or an optionally substituted alkylene chain;
each R⁹ is independently hydrogen, alkyl, haloalkyl, optionally substituted cycloakyl, optionally substituted cycloalkylalkyl, or optionally substituted aryl; and
or two R⁹'s, together with the nitrogen to which they are both attached, form an optionally substituted heterocyclyl;
provided that:
   when X is N, R³ is selected from:

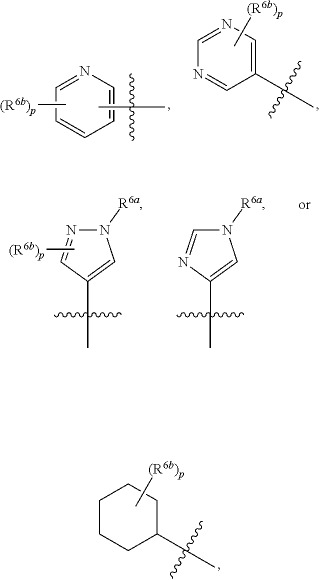

as a stereoisomer, enantiomer, or tautomer thereof or a mixture thereof; or a pharmaceutically acceptable salt, solvate, or prodrug thereof.

In other embodiments, this disclosure is directed to pharmaceutical compositions comprising a pharmaceutically acceptable excipient and a compound of formula (I) or (II), as a stereoisomer, enantiomer, or tautomer thereof or a mixture thereof; or a pharmaceutically acceptable salt, solvate, or prodrug thereof, as described above.

In other embodiments, this disclosure is directed to methods of treating a disease or condition in a mammal modulated by a voltage-gated sodium channel, wherein the methods comprise administering to a mammal in need thereof a therapeutically effective amount of a compound of formula (I) or (II), as a stereoisomer, enantiomer, or tautomer thereof or a mixture thereof; or a pharmaceutically acceptable salt, solvate, or prodrug thereof, as described above.

In other embodiments, this disclosure is directed to methods for the treatment of epilepsy and/or epileptic seizure disorder in a mammal, preferably a human, wherein the methods comprise administering to the mammal in need thereof a therapeutically effective amount of a compound of formula (I) or (II), as set forth above, as a stereoisomer, enantiomer, or tautomer thereof or mixtures thereof; or a pharmaceutically acceptable salt, solvate, or prodrug thereof, or a pharmaceutical composition comprising a therapeutically effective amount of a compound of formula (I) or (II), as set forth above, as a stereoisomer, enantiomer, or tautomer thereof or mixtures thereof, or a pharmaceutically acceptable salt, solvate, or prodrug thereof, and a pharmaceutically acceptable excipient.

In other embodiments, this disclosure is directed to methods of preparing a compound of formula (I) or (II), as set forth above, as a stereoisomer, enantiomer, or tautomer thereof or mixtures thereof; or a pharmaceutically acceptable salt, solvate, or prodrug thereof, or a pharmaceutical composition comprising a therapeutically effective amount of a compound of formula (I) or (II), as set forth above, as a stereoisomer, enantiomer, or tautomer thereof or mixtures thereof, or a pharmaceutically acceptable salt, solvate, or prodrug thereof, and a pharmaceutically acceptable excipient.

In other embodiments, this disclosure is directed to pharmaceutical therapy in combination with one or more other compounds of formula (I) or (II) or one or more other accepted therapies or as any combination thereof to increase the potency of an existing or future drug therapy or to decrease the adverse events associated with the accepted therapy. In one embodiment, this disclosure is directed to a pharmaceutical composition combining a compound of formula (1) or (II) with established or future therapies for the indications listed herein.

### DETAILED DESCRIPTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Compound of the disclosure” or “compounds of the disclosure” refer to compounds of formula (I) or (II), as described above in the Brief Summary, as stereoisomers, enantiomers, or tautomers thereof or mixtures thereof; or pharmaceutically acceptable salts, solvates, or prodrugs thereof.
“Amino” refers to the —NH₂ radical.
“Cyano” refers to the —CN radical.
“Hydroxy” refers to the —OH radical.
“Imino” refers to the =NH substituent.
“Nitro” refers to the —NO₂ radical.
“Oxo” refers to the =0 substituent.
“Thioxo” refers to the =S substituent.
“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂ (where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂(where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms, preferably one to eight carbon atoms and which is attached to the rest of the molecule by a single bond, e.g., ethynyl, propynyl, butynyl, pentynyl, hexynyl, and the like. Unless stated otherwise specifically in the specification, an alkynyl group is optionally substituted by one or more of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), or —S(O)ₜN(R²⁰)₂(where t is 1 to 2), where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂(where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR²⁰, —OC(O)—R²⁰, —N(R²⁰)₂, —C(O)R²⁰, —C(O)OR²⁰, —C(O)N(R²⁰)₂, —N(R²⁰)C(O)OR²², —N(R²⁰)C(O)R²², —N(R²⁰)S(O)ₜR²² (where t is 1 to 2), —S(O)ₜOR²² (where t is 1 to 2), —S(O)ₚR²² (where p is 0 to 2), and —S(O)ₜN(R²⁰)₂(where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R²² is alkyl, haloalkyl, cycloalkyl, cycloalkylalkyl, aryl, aralkyl, heterocyclyl, heterocyclylalkyl, heteroaryl or heteroarylalkyl.

“Aryl” refers to a hydrocarbon ring system radical comprising hydrogen, 6 to 18 carbon atoms and at least one aromatic ring. For purposes of this disclosure, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted by one or more substituents independently selected from the group consisting of alkyl, alkenyl, halo, haloalkyl, haloalkenyl, cyano, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂(where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R_{d}—Rₑ where R_{d} is an alkenylene chain as defined above and R, is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)_{P}R²² (where p is 0 to 2), and —R²¹—S(O)ₜN(R²⁰)₂(where t is 1 to 2) where each R²⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Fused” refers to any ring system described herein which is fused to an existing ring structure in the compounds of the disclosure. When the fused ring system is a heterocyclyl or a heteroaryl, any carbon in the existing ring structure which becomes part of the fused ring system may be replaced with a nitrogen.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Carboxyalkyl” refers to an alkyl radical, as defined above, that is substituted by one or more carboxy radicals. The alkyl part of the carboxyalkyl radical may be optionally substituted as defined above for an alkyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, dioxinyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂(where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“O-heterocyclyl” refers to a heterocycyl radical as defined above containing at least one oxygen atom and no nitrogen atom. An O-heterocyclyl radical may be optionally substituted as described above for heterocyclyl radicals.

“N-heterocyclyl” refers to a heterocyclyl radical as defined above containing at least one nitrogen. An N-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group may be optionally substituted by one or more substituents selected from the group consisting of alkyl, alkenyl, halo, haloalkyl, haloalkenyl, cyano, oxo, thioxo, nitro, thioxo, aryl, aralkyl, cycloalkyl, cycloalkylalkyl, heterocyclyl, heterocyclylalkyl, heteroaryl, heteroarylalkyl, —R²¹—OR²⁰, —R²¹—OC(O)—R²⁰, —R²¹—N(R²⁰)₂, —R²¹—C(O)R²⁰, —R²¹—C(O)OR²⁰, —R²¹—C(O)N(R²⁰)₂, —R²¹—N(R²⁰)C(O)OR²², —R²¹—N(R²⁰)C(O)R²², —R²¹—N(R²⁰)S(O)ₜR²² (where t is 1 to 2), —R²¹—N═C(OR²⁰)R²⁰, —R²¹—S(O)ₜOR²² (where t is 1 to 2), —R²¹—S(O)ₚR²² (where p is 0 to 2), and —R²¹—S(O)ₜN(R²⁰)₂(where t is 1 to 2) where each R²⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R²¹ is independently a direct bond or a straight or branched alkylene or alkenylene chain; and each R²² is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Prodrugs” is meant to indicate a compound that may be converted under physiological conditions or by solvolysis to a biologically active compound of the disclosure. Thus, the term “prodrug” refers to a metabolic precursor of a compound of the disclosure that is pharmaceutically acceptable. A prodrug may be inactive when administered to a subject in need thereof, but is converted in vivo to an active compound of the disclosure. Prodrugs are typically rapidly transformed in vivo to yield the parent compound of the disclosure, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et a/., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the disclosure in vivo when such prodrug is administered to a mammalian subject. Prodrugs of a compound of the disclosure may be prepared by modifying functional groups present in the compound of the disclosure in such a way that the modifications are cleaved, either in routine manipulation or in vivo, to the parent compound of the disclosure. Prodrugs include compounds of the disclosure wherein a hydroxy, amino or mercapto group is bonded to any group that, when the prodrug of the compound of the disclosure is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the disclosure and the like.

“Stable compound” and “stable structure” are meant to indicate a compound that is sufficiently robust to survive isolation to a useful degree of purity from a reaction mixture, and formulation into an efficacious therapeutic agent.

As used herein, a “floating bond” or a bond not shown to be directly bound to a specific atom of a molecule may be attached at any substitutable point of the radical or molecule to which it is floating over. An exemplary floating bond is shown on the radical below:

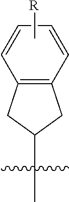

In the structure above, R may be attached to any of the substitutable positions of the radical. For example, R may be covalently bound to any of the positions α-g as shown below:

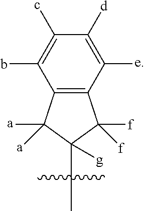

“Mammal” includes humans and both domestic animals such as laboratory animals and household pets, (e.g., cats, dogs, swine, cattle, sheep, goats, horses, rabbits), and non-domestic animals such as wildelife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution (“unsubstituted). When a functional group is described as “optionally substituted,” and in turn, substituents on the functional group are also “optionally substituted” and so on, for the purposes of this disclosure, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the disclosure. As used herein, the term “solvate” refers to an aggregate or solid form that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compound of the disclosure may be true solvates, while in other cases; the compound of the disclosure may merely retain adventitious water or be a mixture of water plus some adventitious solvent.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“Seizure disorders” refers to seizures and disorders associated with seizures such as partial onset (focal) seizures, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures+, Rett syndrome, multiple sclerosis, Alzheimer's disease, autism, ataxia, hypotonia and paroxysmal dyskinesia.

Preferably, the term “seizure disorder” refers to partial onset (focal) epilepsy. “Therapeutically effective amount” refers to a range of amounts of a compound of the disclosure, which, upon administration to a human, treats, ameliorates or prevents a seizure disorder, preferably epilepsy, in the human, or exhibits a detectable therapeutic or preventative effect in the human having a seizure disorder. The effect is detected by, for example, a reduction in seizures (frequency) or by the severity of seizures (quality). The precise therapeutically effective amount for a given human will depend upon the human's size and health, the nature and extent of the seizure disorder, the presence of any concomitant medications, and other variables known to those of skill in the art. The therapeutically effective amount for a given situation is determined by routine experimentation and is within the judgment of the clinician.

“Treatment” refers to therapeutic applications to slow or stop progression of a seizure disorder, prophylactic application to prevent development of a seizure disorder, and/or reversal of a seizure disorder. Reversal of a seizure disorder differs from a therapeutic application which slows or stops a seizure disorder in that with a method of reversing, not only is progression of a seizure disorder completely stopped, cellular behavior is moved to some degree toward a normal state that would be observed in the absence of the seizure disorder.

“Treating” or “treatment” as used herein covers the treatment of the disease or condition of interest in a mammal, preferably a human, having the disease or condition of interest, and includes:
(a) preventing the disease or condition from occurring in a mammal, in particular, when such mammal is predisposed to the condition but has not yet been diagnosed as having it;
(b) inhibiting the disease or condition, i.e., arresting its development;
(c) relieving (or ameliorating) the disease or condition, i.e., causing regression of the disease or condition; or
(d) relieving (or ameliorating) the symptoms resulting from the disease or condition, i.e., relieving a seizure disorder without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of this disclosure may contain at least one asymmetric carbon atom and thus may exist as racemates, enantiomers, and/or diastereoisomers. For the present disclosure, the words diastereomer and diastereoisomer and related terms are equivalent and interchangeable. Unless otherwise indicated, this disclosure includes all enantiomeric and diastereoisomeric forms of the compounds of formula (I) or (II). Pure stereoisomers, mixtures of enantiomers and/or diastereoisomers, and mixtures of different compounds of the disclosure are included herein. Thus, compounds of formula (1) or (II) may occur as racemates, racemic or diastereoisomeric mixtures and as individual diastereoisomers, or enantiomers, unless a specific stereoisomer enantiomer or diastereoisomer is identified, with all isomeric forms being included in the present disclosure. For this disclosure, a racemate or racemic mixture implies a 50:50 mixture of stereoisomers only. Other enantiomerically or diastereomerically enriched mixtures of varying ratios of stereoisomers are also contemplated.

“Enantiomers” refer to asymmetric molecules that can exist in two different isomeric forms which have different configurations in space. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane-polarized light in different directions). Because they do not have a plane of symmetry, enantiomers are not identical with their mirror images; molecules which exist in two enantiomeric forms are chiral, which means that they can be regarded as occurring in “left” and “right” handed forms. The most common cause of chirality in organic molecules is the presence of a tetrahedral carbon bonded to four different substituents or groups. Such a carbon is referred to as a chiral center, or stereogenic center.

Enantiomers have the same empirical chemical formula, and are generally chemically identical in their reactions, their physical properties, and their spectroscopic properties. However, enantiomers show different chemical reactivity toward other asymmetric compounds, and respond differently toward asymmetric physical disturbances. The most common asymmetric disturbance is polarized light.

An enantiomer can rotate plane-polarized light; thus, an enantiomer is optically active. Two different enantiomers of the same compound will rotate plane-polarized light in the opposite direction; thus, the light can be rotated to the left or counterclockwise for a hypothetical observer (this is levarotatory or “I”, or minus or “−”) or it can be rotated to the right or clockwise (this is dextrorotatory or “d” or plus or “+”). The sign of optical rotation (+) or (−), is not related to the R,S designation. A mixture of equal amounts of two chiral enantiomers is called a racemic mixture, or racemate, and is denoted either by the symbol (+/−) or by the prefix “d,l” to indicate a mixture of dextrorotatory and levorotatory forms. Racemates or racemic mixtures show zero optical rotation because equal amounts of the (+) and (−) forms are present. In general, the presence of a single enantiomer rotates polarized light in only one direction; thus, a single enantiomer is referred to as optically pure.

The designations “R” and “S” are used to denote the three-dimensional arrangement of atoms (or the configuration) of the stereogenic center. The designations may appear as a prefix or as a suffix; they may or may not be separated from the enantiomer name by a hyphen; they may or may not be hyphenated; and they may or may not be surrounded by parentheses. A method for determining the designation is to refer to the arrangement of the priority of the groups at the stereogenic center when the lowest priority group is oriented away from a hypothetical observer: If the arrangement of the remaining three groups from the higher to the lower priority is clockwise, the stereogenic center has an “R” configuration; if the arrangement is counterclockwise, the stereogenic center has an “S” configuration.

“Resolution” or “resolving” when used in reference to a racemic compound or mixture refers to the separation of a racemate into its two enantiomeric forms (i.e., (+) and (−); (R) and (S) forms).

“Enantiomeric excess” or “ee” refers to a product wherein one enantiomer is present in excess of the other, and is defined as the absolute difference in the mole fraction of each enantiomer. Enantiomeric excess is typically expressed as a percentage of an enantiomer present in a mixture relative to the other enantiomer. For purposes of this disclosure, the (S)-enantiomer of a compound prepared by the methods disclosed herein is considered to be “substantially free” of the corresponding (R)-enantiomer when the (S)-enantiomer is present in enantiomeric excess of greater than 80%, preferably greater than 90%, more preferably greater than 95% and most preferably greater than 99%.

Certain compounds have been labeled with “P1”, “P2”, et seq. or “D1”, “D2”, et seq. This demarcation indicates a compound is a first eluting peak (i.e., P1) from a chiral separation technique and does not necessarily indicate a specific stereochemistry.

A “tautomer” refers to a proton shift from one atom of a molecule to another atom of the same molecule. The present disclosure includes tautomers of any compound of formula (I) or (II) as described herein.

The use of parentheses and brackets in substituent groups may be used herein to conserve space. Accordingly, the use of parenthesis in a substituent group indicates that the group enclosed within the parentheses is attached directly to the atom preceding the parenthesis. The use of brackets in a substituent group indicates that the group enclosed within the brackets is also attached directly to the atom preceding the parenthesis.

For example, a compound of formula (I) or (II) wherein a compound having the following structure:

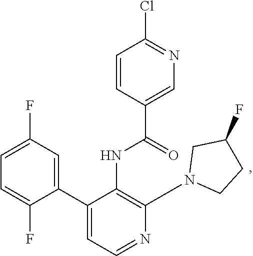 [SMILES: O=C(Nc1c(-c2cc(F)ccc2F)ccnc1N1CC[C@H](F)C1)c1ccc(Cl)nc1]

is named herein as (S)-6-chloro-N-(4-(2,5-difluorophenyl)-2-(3-fluoropyrrolidin-1-yl)pyridin-3-yl)nicotinamide.

### Compounds

One embodiment of the disclosure is compounds of formula (1) or (II), as set forth above in the Brief Summary, as individual stereoisomers, enantiomers, or tautomers thereof or as mixtures thereof; or pharmaceutically acceptable salts, solvates, or prodrugs thereof. That is, one embodiment provides a compound of formula (I):

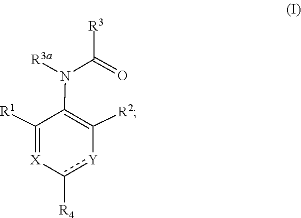

wherein:
[symbol] represents a double or single bond such that all valences are satisfied;
Y is N or NR^{4a};
X is C(R⁷) or N;
R¹ is selected from:

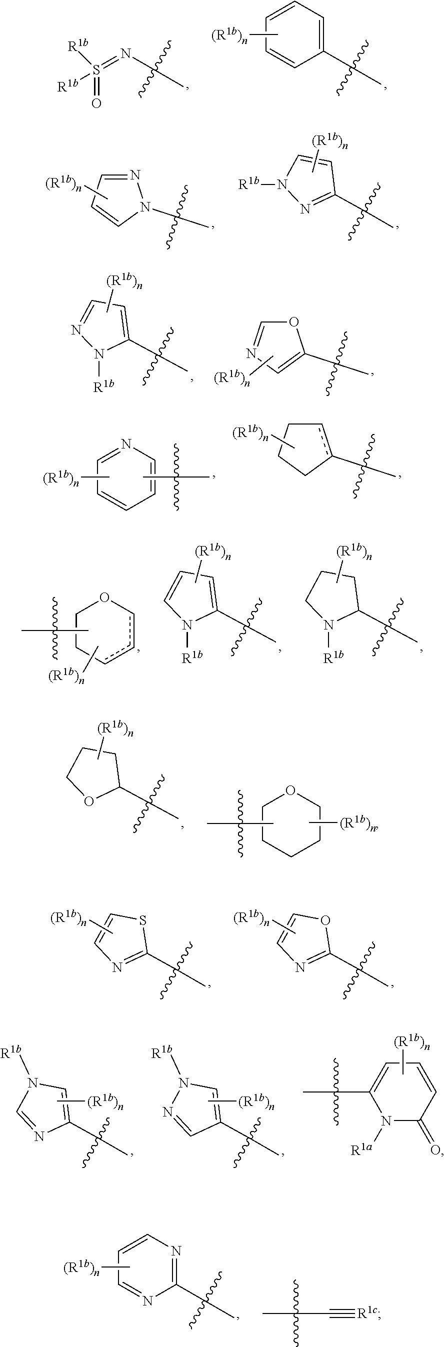

wherein:
each occurrence of —independently represents a double or single bond such
that all valences are satisfied; [symbol]
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
R^{1c} is N or —Si(CH₃)₃;
R² is selected from:

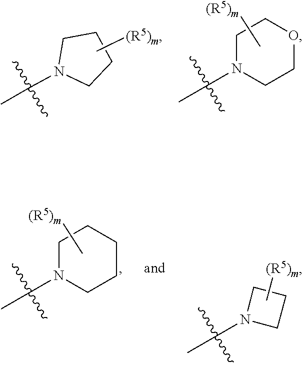

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain;
R³ is alkyl, —R⁸—N(R⁹)₂, —R⁸—OR⁹, or
R³ is selected from:

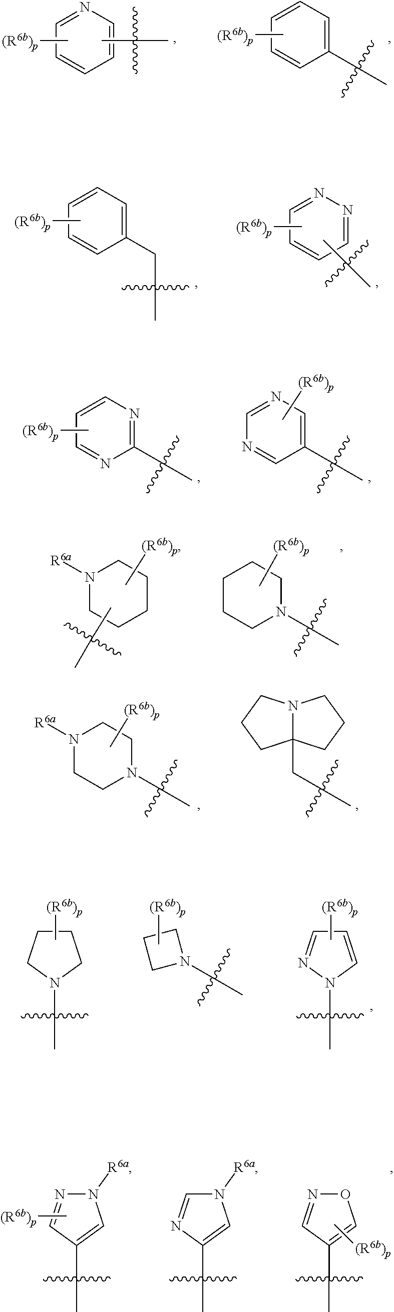

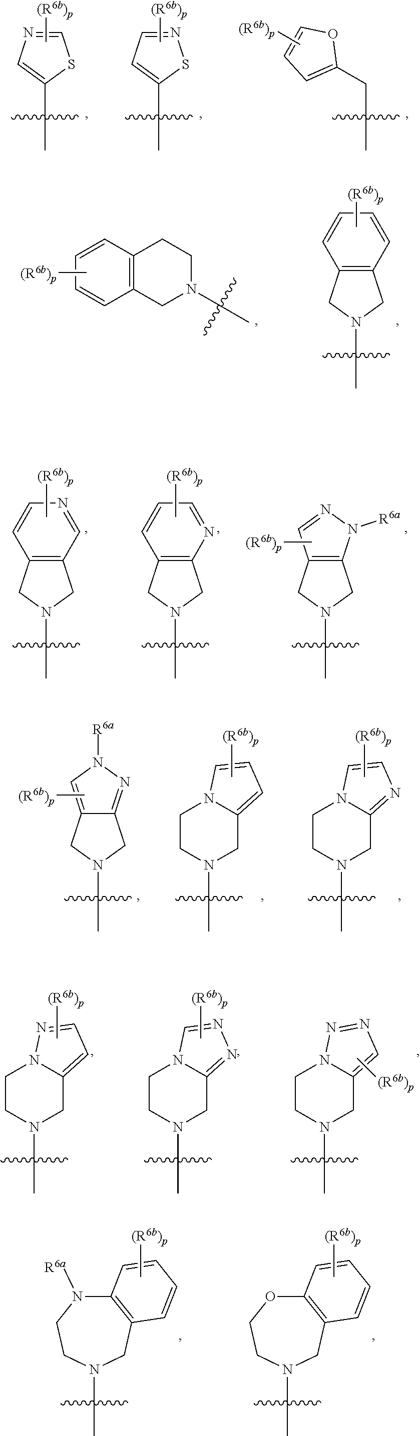

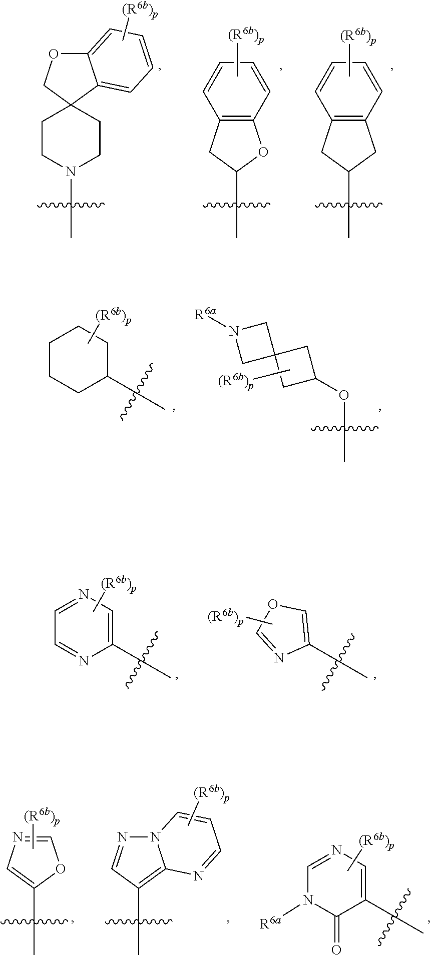

wherein:
p is 0, 1, 2, 3, 4, or 5; R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain;
R^{3a} is hydrogen or alkyl;
R⁴ is hydrogen, alkyl, —R⁸—OR⁹, halo, haloalkyl, or cyano; or R⁴ together with the carbon to which it is attached, joins with R^{4a} together with the nitrogen to which it is attached to form an optionally substituted 5-membered N-heteroaryl;
R⁷ is hydrogen, alkyl, halo, or —R⁸—OR⁹;
each R⁸ is independently a direct bond or an optionally substituted alkylene chain;
each R⁹ is independently hydrogen, alkyl, haloalkyl, carboxyalkyl, optionally substituted cycloalkyl, optionally substituted cycloalkylalkyl, or optionally substituted aryl; and
or two R⁹'s, together with the nitrogen to which they are both attached, form an optionally substituted heterocyclyl;
provided that:
   when X is N, R³ is selected from:

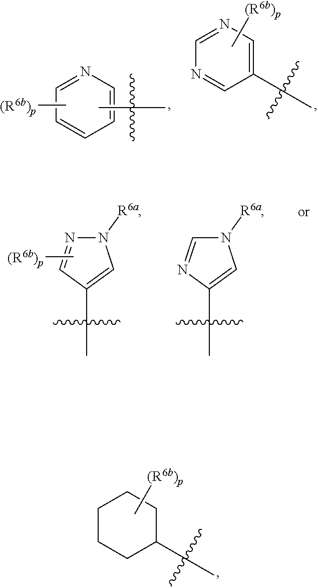

as a stereoisomer, enantiomer, or tautomer thereof or a mixture thereof; or a pharmaceutically acceptable salt, solvate, or prodrug thereof.
Certain embodiments provide a compound of formula (II):

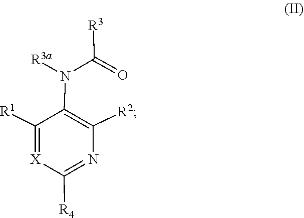

wherein:
X is C(R⁷) or N;
R¹ is selected from:

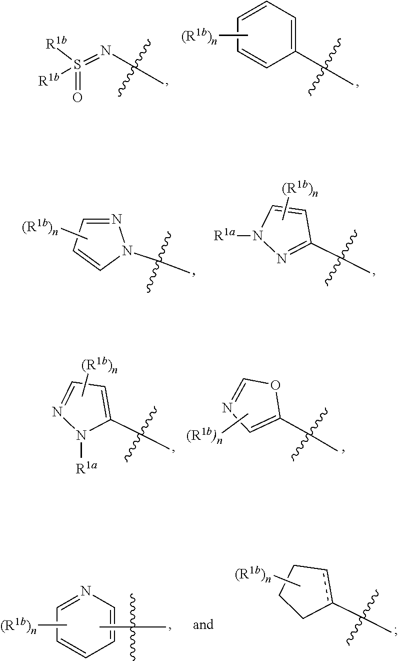

wherein:
[symbol] represents a double or single bond;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl;
R² is selected from:

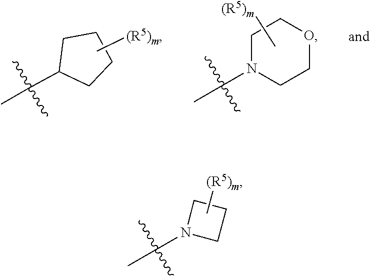

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted cycloalkyl; and
or two R⁵'s, together with the carbons to which they are attached, form an optionally substituted alkylene chain;
R³ is alkyl, —R⁸—N(R⁹)₂, —R⁸—OR⁹, or
R³ is selected from:

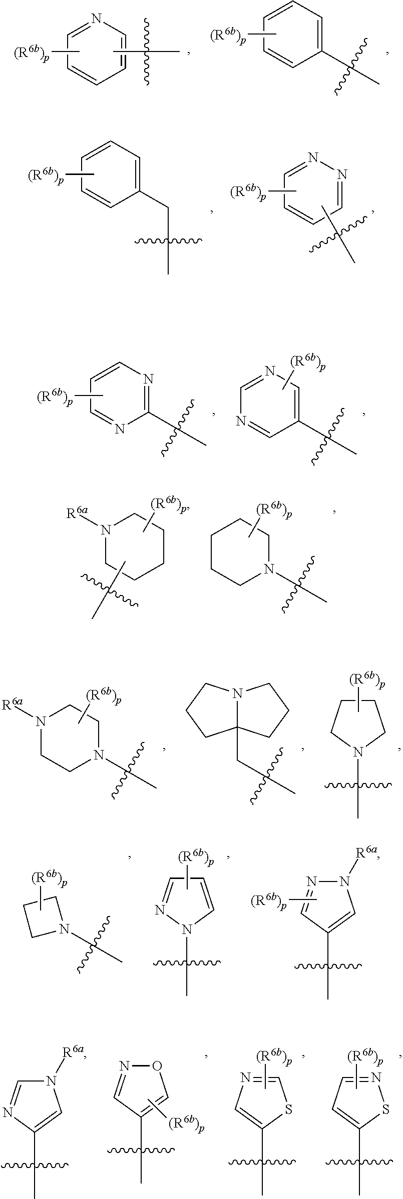

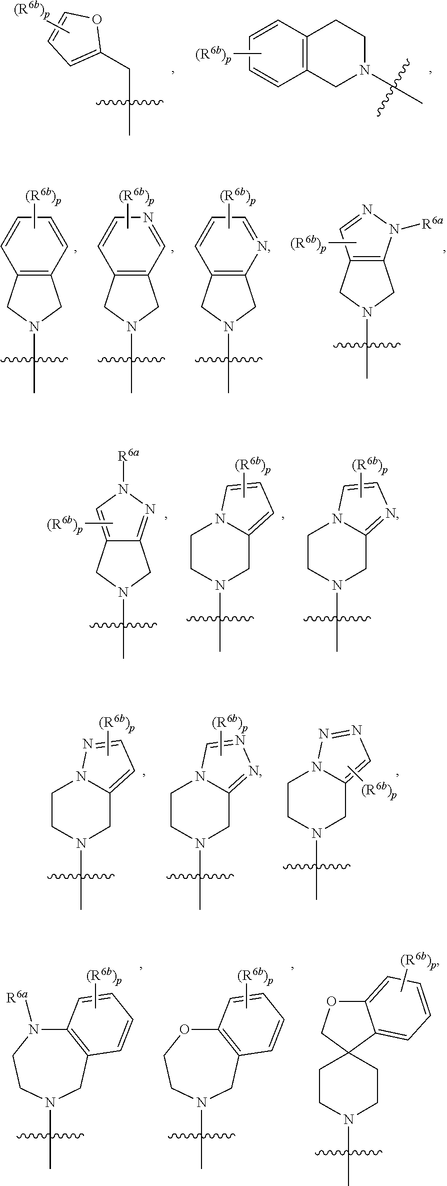

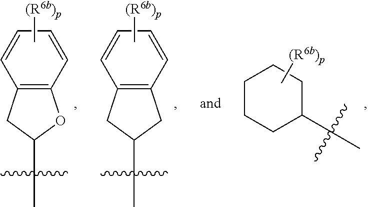

wherein:
p is 0,1,2,3,4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, —R⁸—C(═O)N(R⁹)₂,optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain;
R^{3a} is hydrogen or alkyl;
R⁴ is hydrogen, alkyl, —R⁸—OR⁹, halo, haloalkyl, or cyano,
R⁷ is hydrogen, alkyl, halo, or —R⁸—OR⁹;
each R³ is independently a direct bond or an optionally substituted alkylene chain;
each R⁹ is independently hydrogen, alkyl, haloalkyl, optionally substituted cycloakyl, optionally substituted cycloalkylalkyl, or optionally substituted aryl; and
or two R⁹'s, together with the nitrogen to which they are both attached, form an optionally substituted heterocyclyl;
provided that:
   when X is N, R³ is selected from:

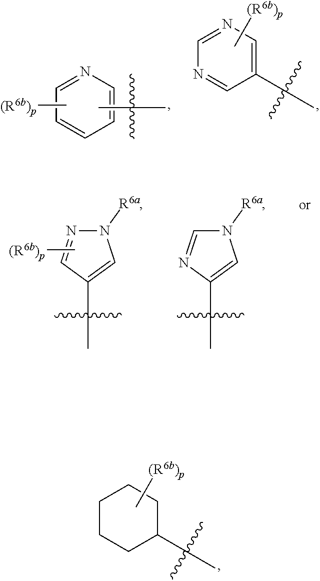

as a stereoisomer, enantiomer, or tautomer thereof or a mixture thereof; or a pharmaceutically acceptable salt, solvate, or prodrug thereof.

In some embodiments, X is C(R⁷). In certain embodiments, X is C(R⁷) and R⁷ is hydrogen. In some specific embodiments, X is C(R⁷) and R⁷ is halo. In certain more specific embodiments, X is C(R⁷) and R⁷ is fluoro. In some specific embodiments, X is N.

In some embodiments, the compound has the following formula (Ia):

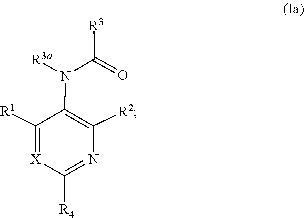

X, R¹, R², R³, R^{3a}, and R⁴ are each as defined above in the Brief Description; as a stereoisomer, enantiomer, or tautomer thereof or a mixture thereof; or a pharmaceutically acceptable salt, solvate, or prodrug thereof.

In certain embodiments, the compound has the following formula (Ib):

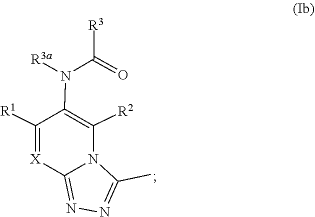

X, R¹, R², R³, R^{3a}, and R⁴ are each as defined above in the Brief Description;

as a stereoisomer, enantiomer, or tautomer thereof or a mixture thereof; or a pharmaceutically acceptable salt, solvate, or prodrug thereof.

In some embodiments, R¹ is selected from:

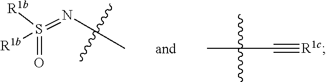

wherein:
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹;
R^{1c} is N or —Si(CH₃)₃.

In certain embodiments, R¹ is selected from:

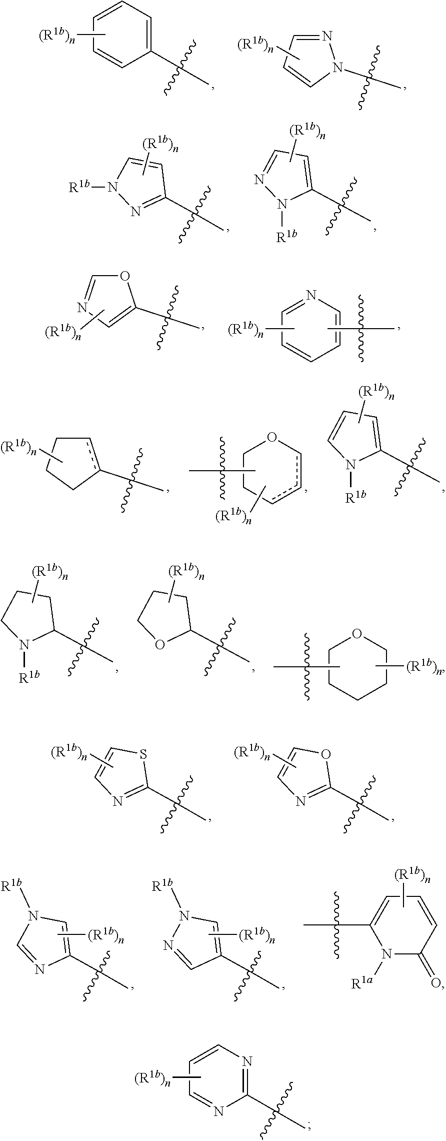

wherein:
each occurrence of [symbol] independently represents a double or single bond such
that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl.

In certain embodiments, R¹ is selected from:

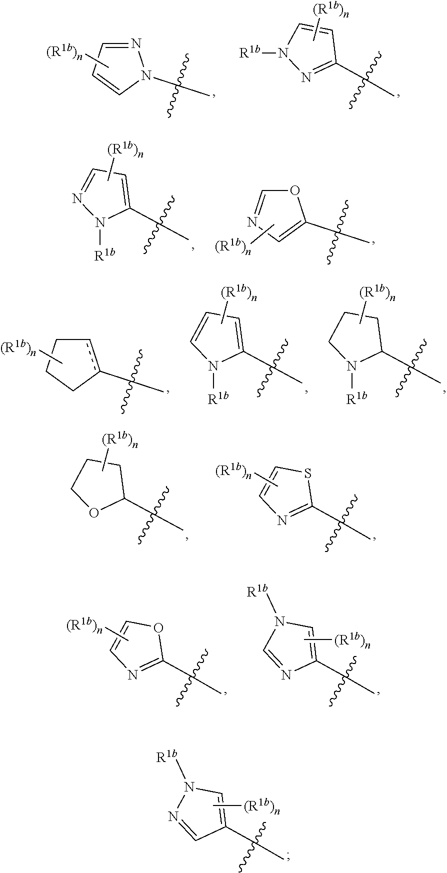

wherein: [symbol]
each occurrence of independently represents a double or single bond such
that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl.

In some embodiments, R¹ is selected from:

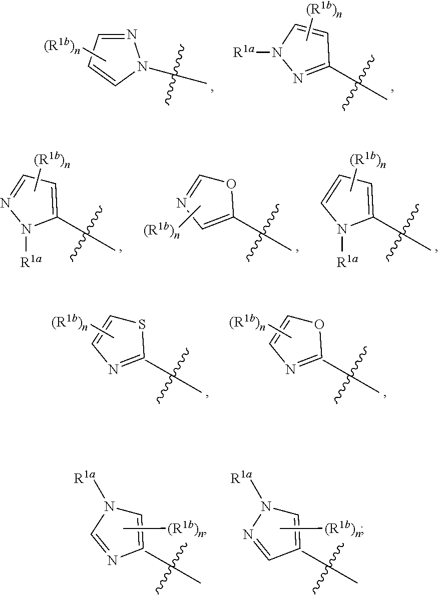

wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl.

In certain embodiments, R¹ is selected from:

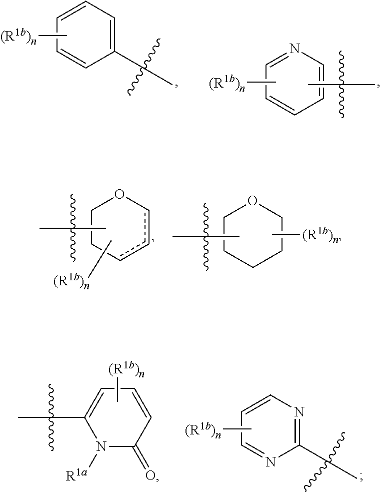

wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl.

In some embodiments, R¹ is selected from:

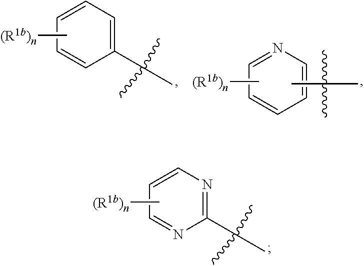

wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl.

In certain embodiments, R¹ is:

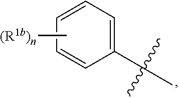

wherein:
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In some embodiments, R¹ has one of the following structures:

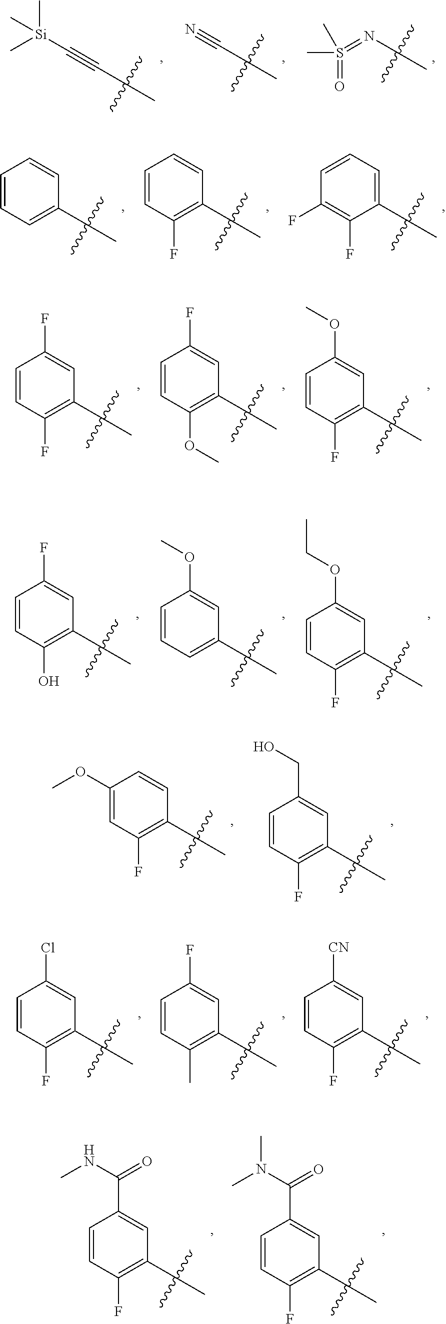

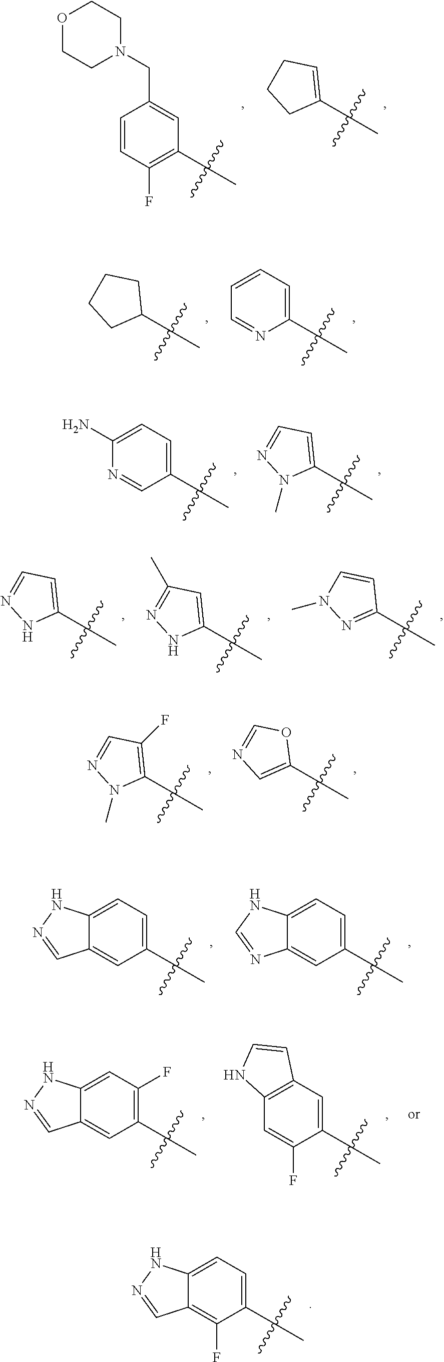

In some embodiments, R¹ has the following structure:

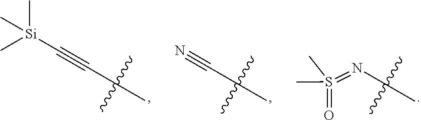

In certain embodiments, R¹ has one of the following structures:

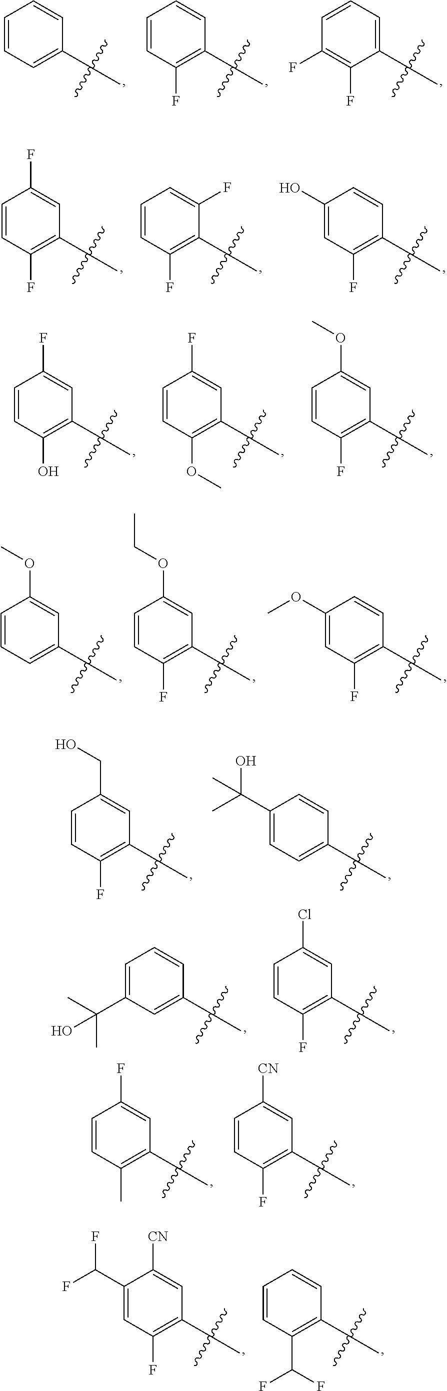

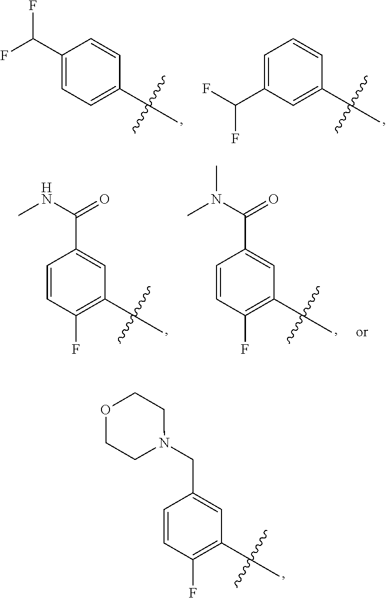

In some embodiments, R¹ has one of the following structures:

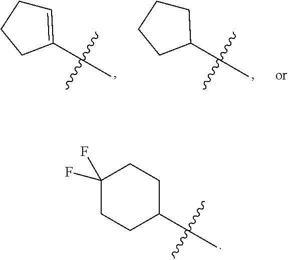 [SMILES: *C1=CCCC1 ; *C1CCCC1 ; *C1CCC(F)(F)CC1]

In some embodiments, R¹ has one of the following structures:

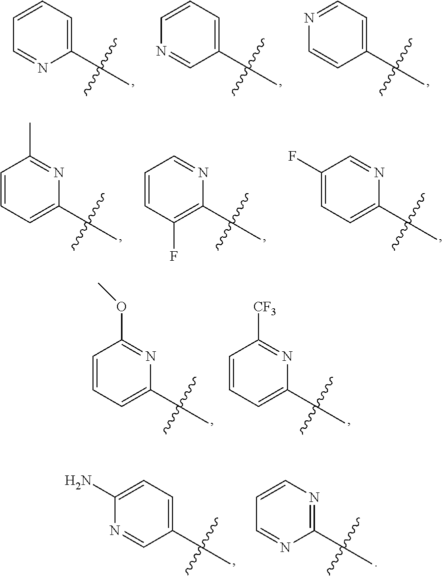

In certain embodiments, R¹ has one of the following structures:

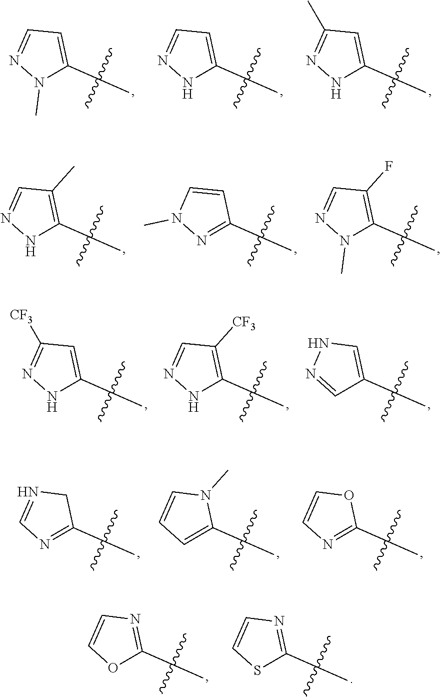

In some embodiments, R¹ has one of the following structures:

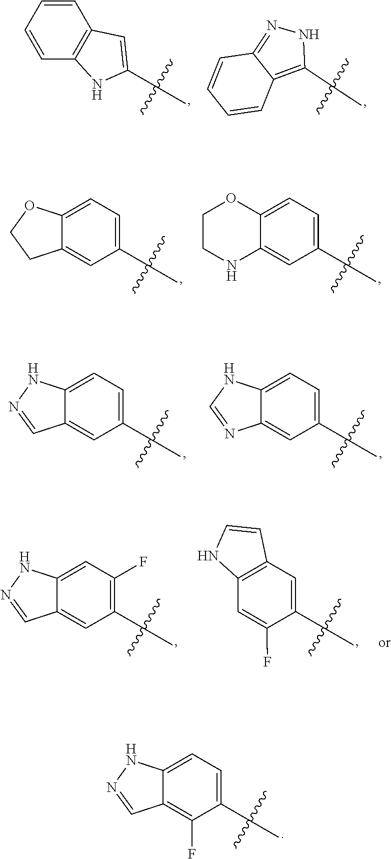

In some embodiments, R¹ is selected from:

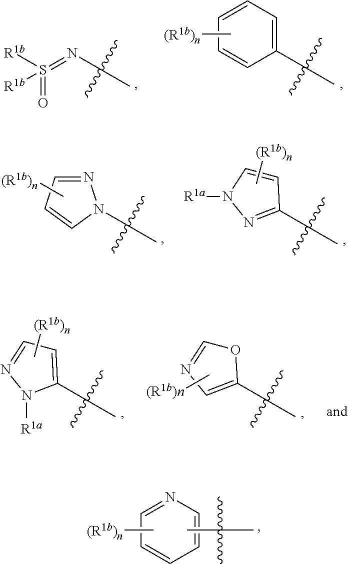

wherein:
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl

In some more specific embodiments, R¹ is selected from:

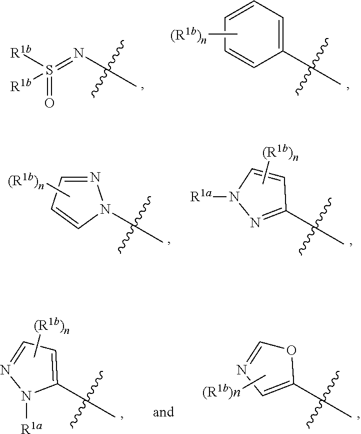

wherein:
n is 0, 1, 2, 3, 4, or 5;
R^{1′} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In certain embodiments, R¹ is selected from:

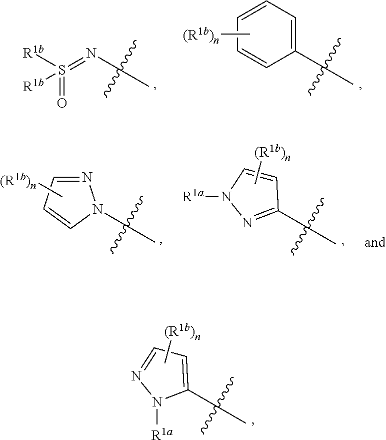

wherein:
n is 0, 1, 2, 3, 4, or 5; R^{1′} is hydrogen, or alkyl;
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In some more specific embodiments, R¹ is selected from:

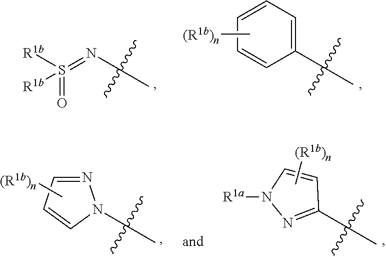

wherein:
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In certain more specific embodiments, R¹ is selected from:

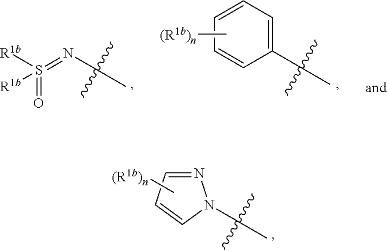

wherein:
n is 0, 1, 2, 3, 4, or 5; R^{1′} is hydrogen, or alkyl;
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In some embodiments, R¹ is selected from:

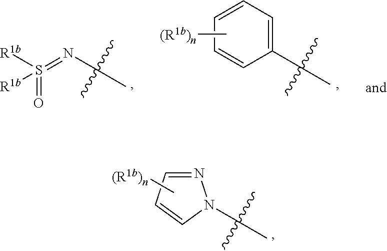

wherein:
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In certain embodiments, R¹ is:

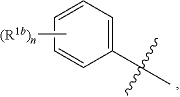

wherein:
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹;
or two R^{1b}'s attached to adjacent carbons, together with the carbons to which they are attached, form an optionally substituted N-heteroaryl.

In certain embodiments, R¹ is:

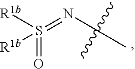

wherein:
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹.

In some more specific embodiments, R¹ is:

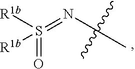

wherein:
each R^{1b} is independently alkyl.

In more specific embodiments, R¹ has one of the following structures:

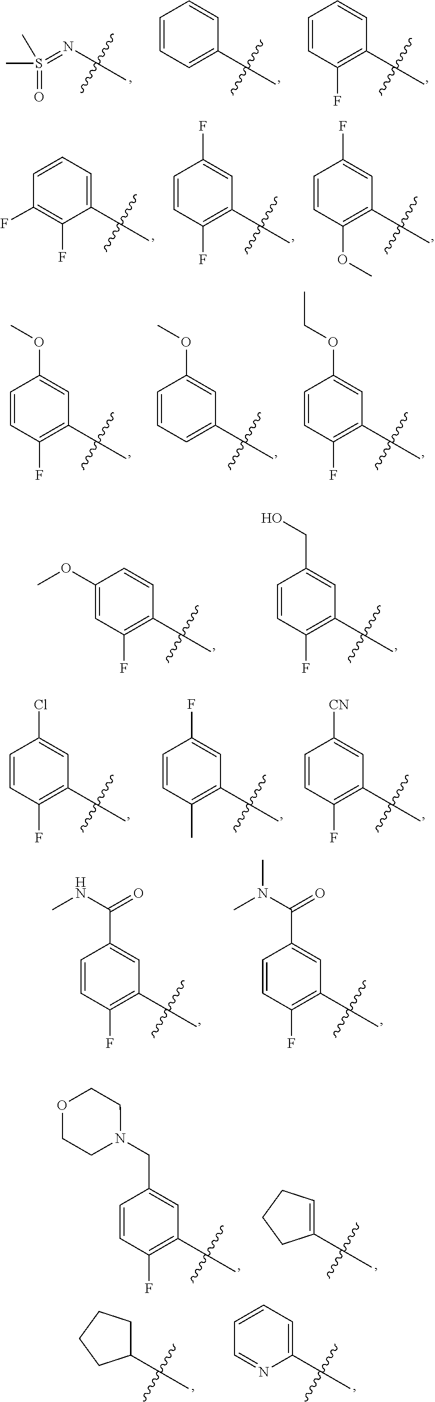

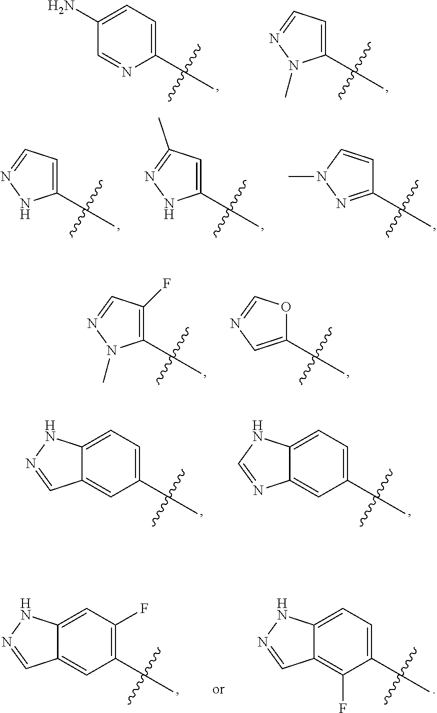

In some specific embodiments, R¹ has the following structure:

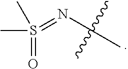 [SMILES: *N=S(C)(C)=O]

In certain specific embodiments, R¹ has one of the following structures:

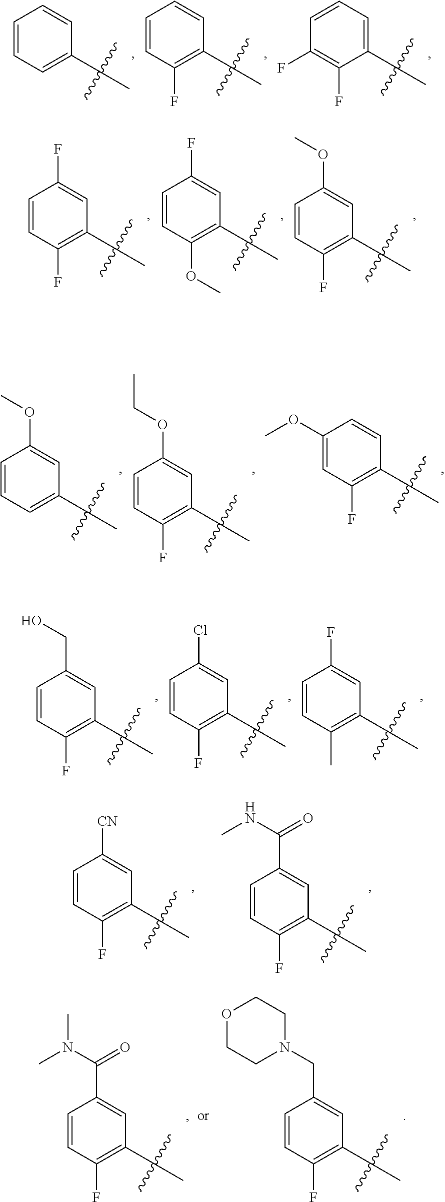

In some embodiments, R¹ has one of the following structures:

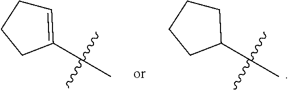 [SMILES: *C1=CCCC1 ; *C1CCCC1]

In certain embodiments, R¹ has one of the following structures:

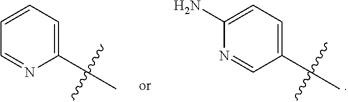 [SMILES: *c1ccccn1 ; *c1ccc(N)nc1]

In some specific embodiments, R¹ has one of the following structures:

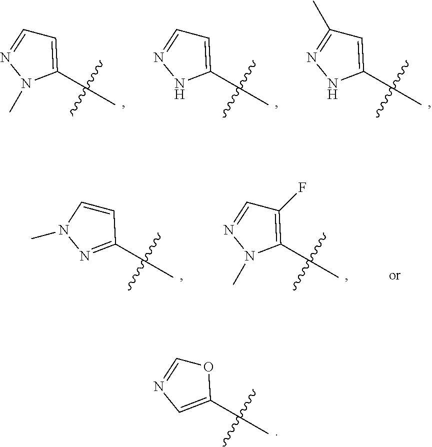

In certain specific embodiments, R¹ has one of the following structures:

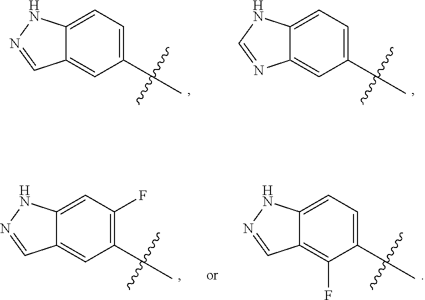 [SMILES: *c1ccc2[nH]ncc2c1 ; *c1ccc2[nH]cnc2c1 ; *c1cc2cn[nH]c2cc1F ; "or" ; *c1ccc2[nH]ncc2c1F]

In some embodiments, R² is selected from:

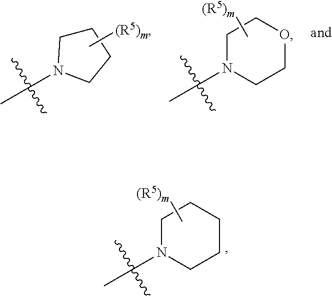

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain.

In some embodiments, R² is selected from:

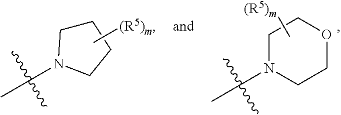

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain.

In some embodiments, R² is:

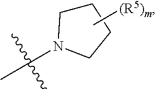

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain.

In some embodiments, R² is selected from:

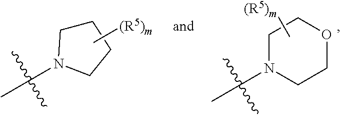

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted cycloalkyl; and
or two R⁵'s, together with the carbons to which they are attached, form an optionally substituted alkylene chain.

In certain embodiments, R² is:

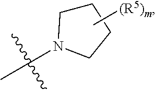

wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted cycloalkyl; and
or two R⁵'s, together with the carbons to which they are attached, form an optionally substituted alkylene chain.

In more specific embodiments, R² has one of the following structures:

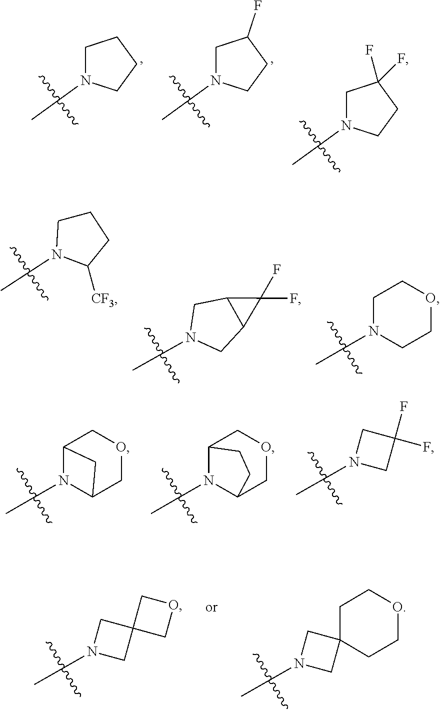

In some embodiments, R² has one of the following structures:

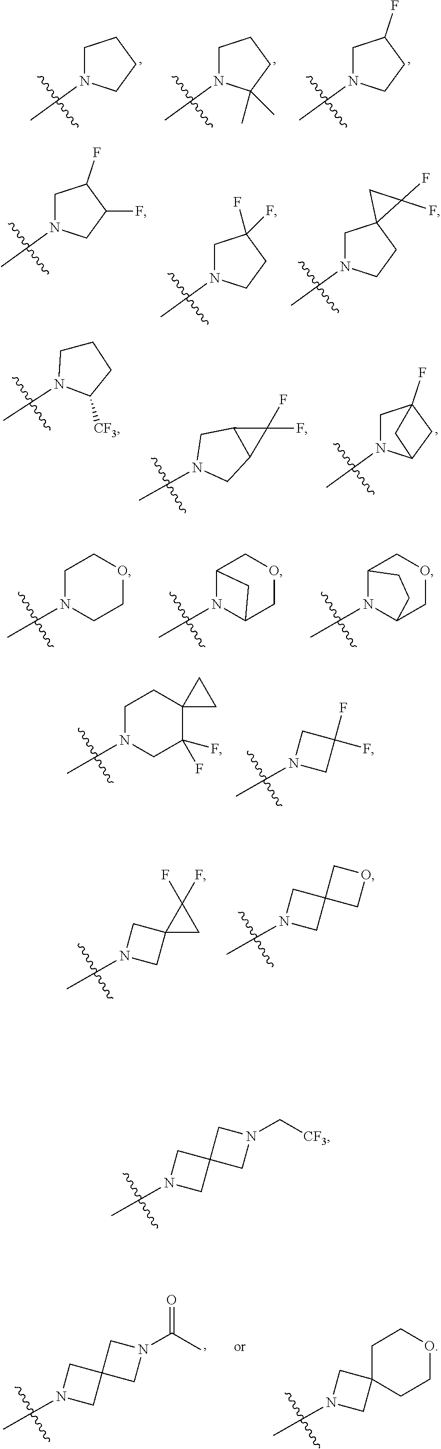

In certain embodiments, R² has one of the following structures:

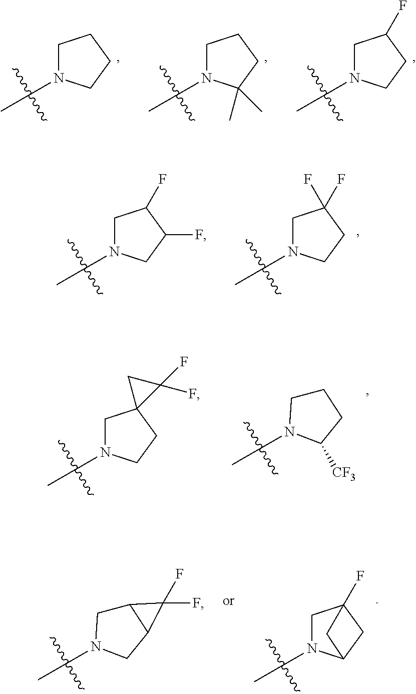

In some embodiments, R² has one of the following structures:

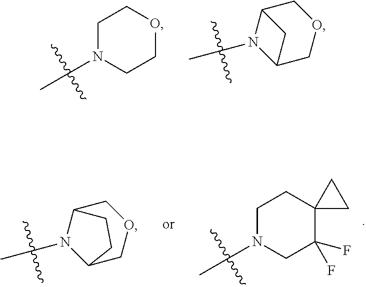 [SMILES: *N1CCOCC1 ; *N1C2COCC1C2 ; *N1C2CCC1COC2 ; *N1CCC2(CC2)C(F)(F)C1 ; "."]

In some embodiments, R² has one of the following structures:

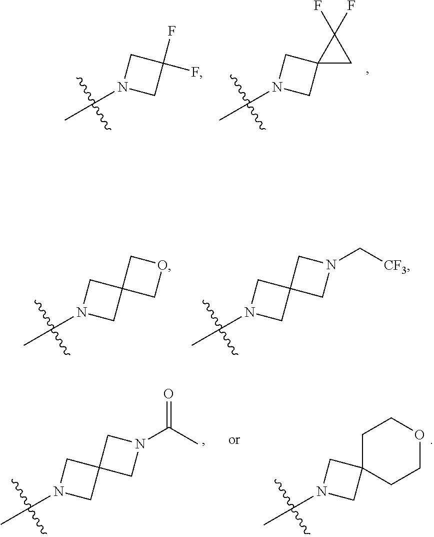

In some embodiments, R² has one of the following structures:

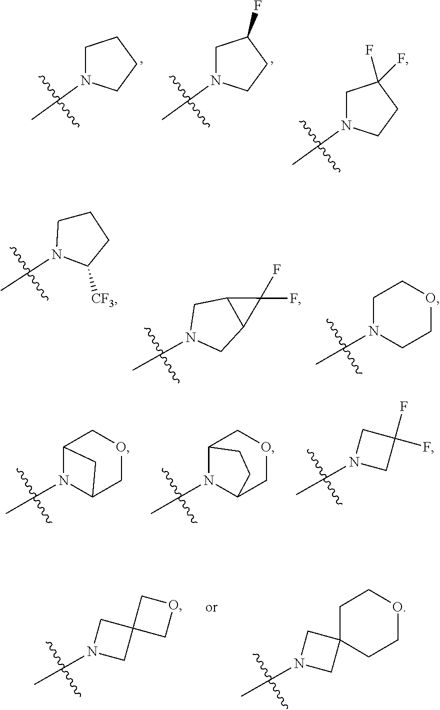

In some embodiments, R² has one of the following structures:

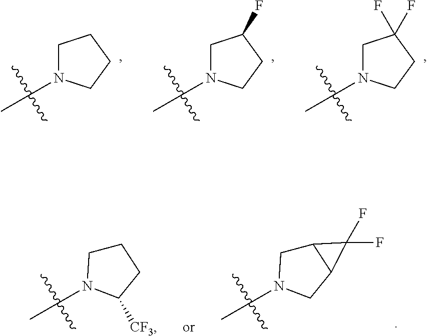 [SMILES: *N1CCCC1 ; *N1CC[C@H](F)C1 ; *N1CCC(F)(F)C1 ; *N1CCC[C@@H]1C(F)(F)F ; *N1CC2C(C1)C2(F)F ; "."]

In some embodiments, R² has one of the following structures:

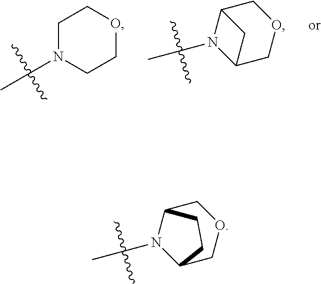 [SMILES: *N1CCOCC1 ; *N1C2COCC1C2 ; *N1[C@@H]2CC[C@H]1COC2]

In some embodiments, R² has one of the following structures:

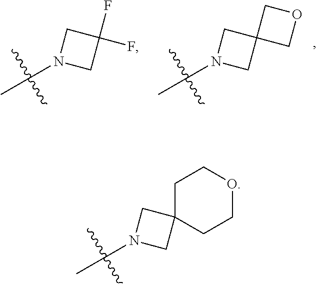 [SMILES: *N1CC(F)(F)C1 ; *N1CC2(COC2)C1 ; "," ; *N1CC2(CCOCC2)C1]

In certain embodiments, R³ is selected from:

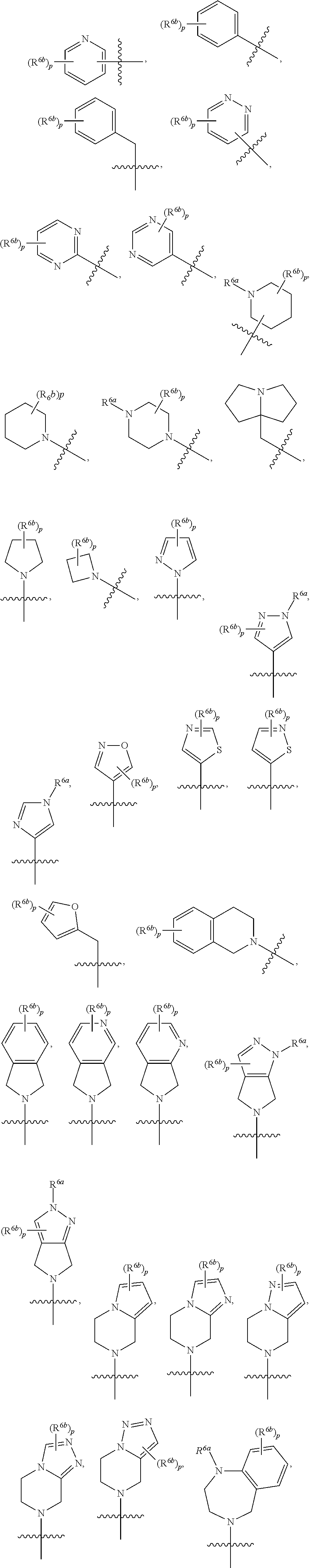

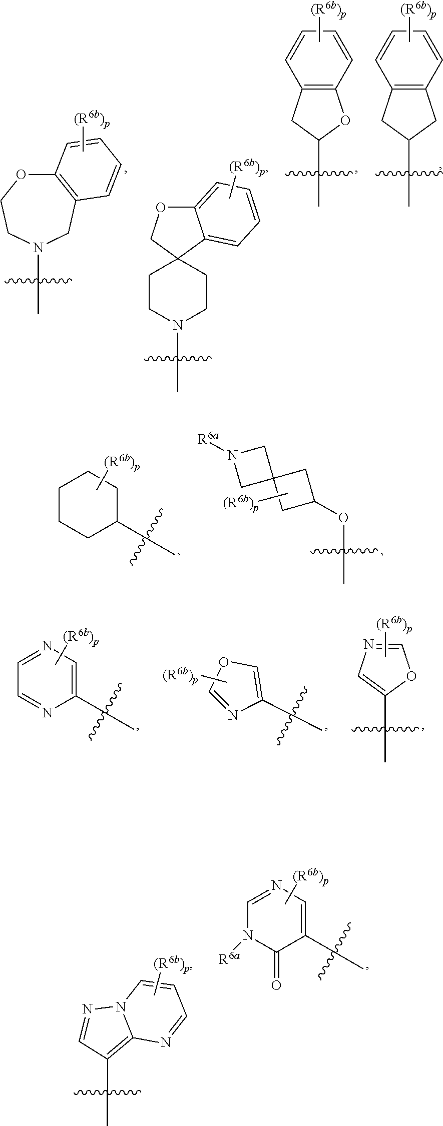

R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain;
or an occurrence of R^{6b} and an occurrence of R^{1b} join to form an optionally substituted alkylene chain.

In some embodiments, R³ is alkyl, —R⁸—N(R⁹)₂, or —R⁸—OR⁹. In certain embodiments, R³ is selected from:

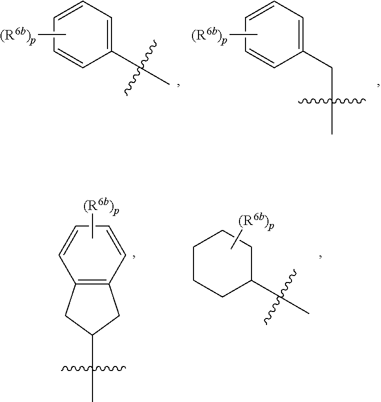

wherein:
p is 0, 1, 2, 3, 4, or 5;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain.

In some embodiments, R³ is selected from:

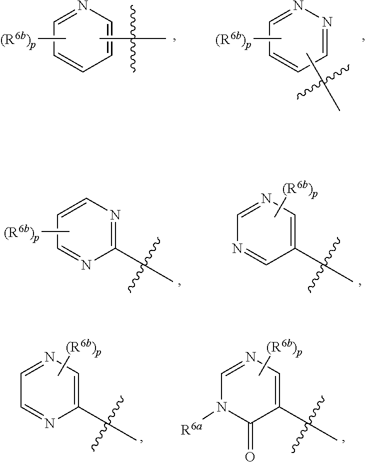

wherein:
p is 0, 1, 2, 3, 4, or 5;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain.

In certain embodiments, R³ is selected from:

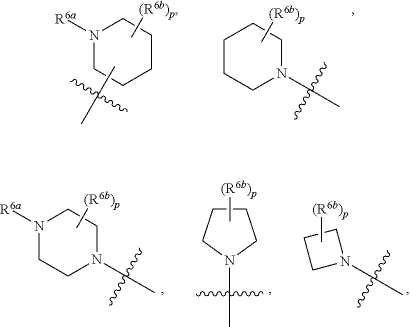

wherein:
p is 0,1,2,3,4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain.

In some embodiments, R³ is selected from:

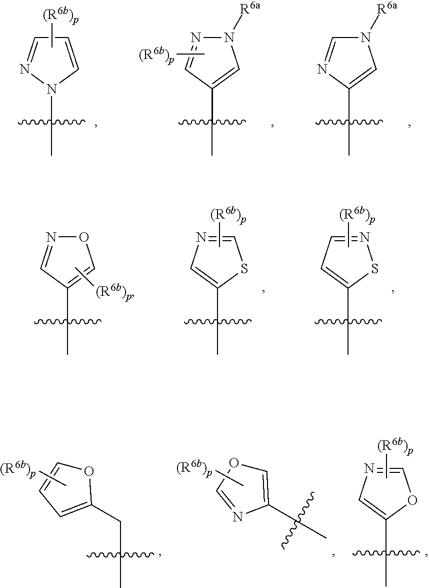

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain.

In certain embodiments, R³ is selected from:

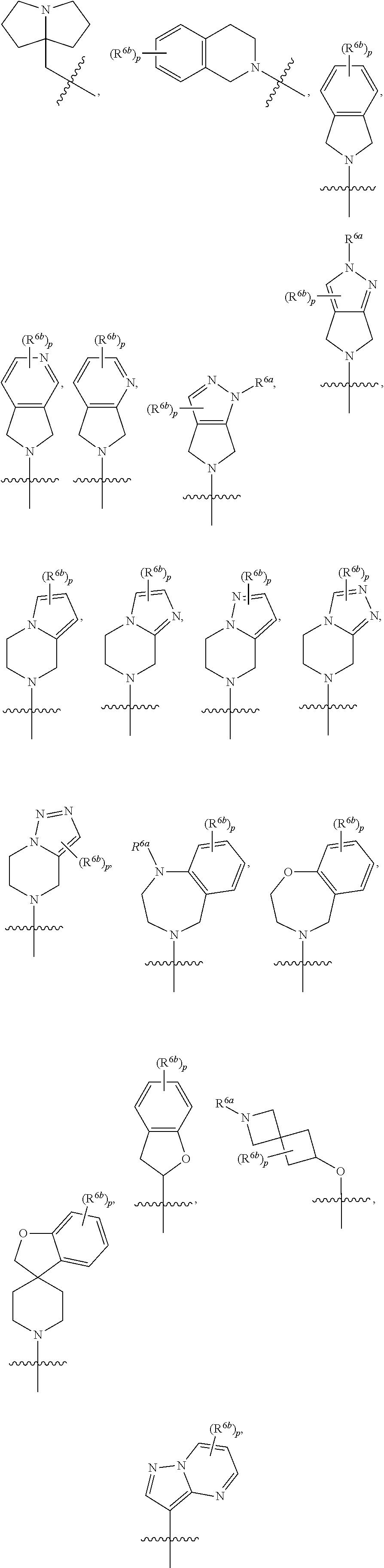

wherein:
p is 0, 1, 2, 3, 4, or 5;
R⁶² is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain; or an occurrence of R^{6b} and an occurrence of R^{1b} join to form an optionally substituted alkylene chain.

In some embodiments, R³ has one of the following structures:

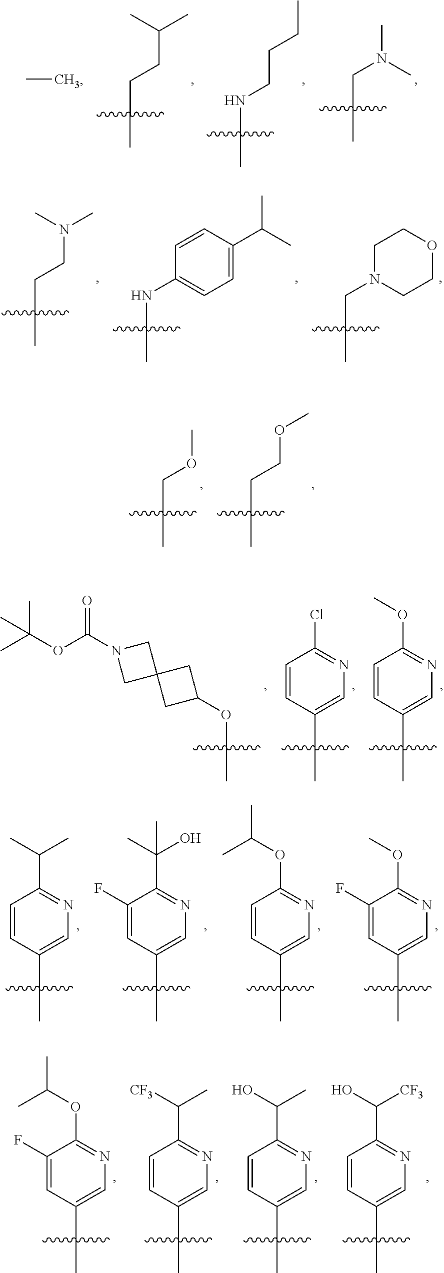

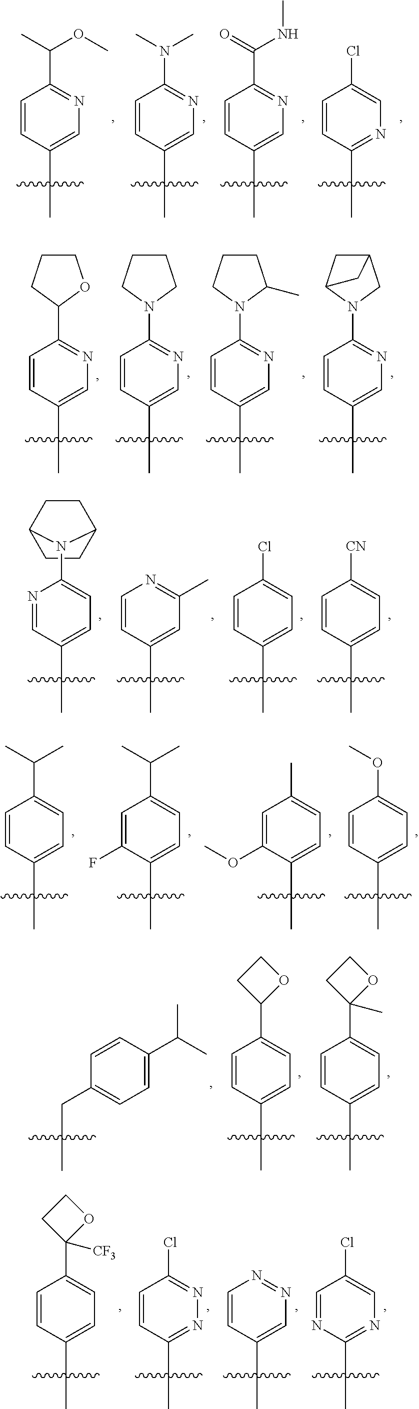

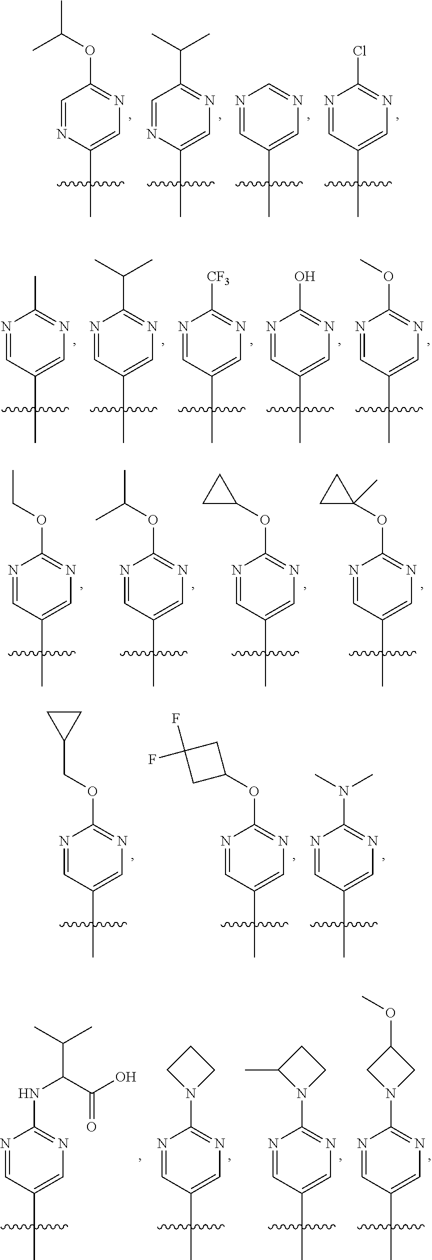

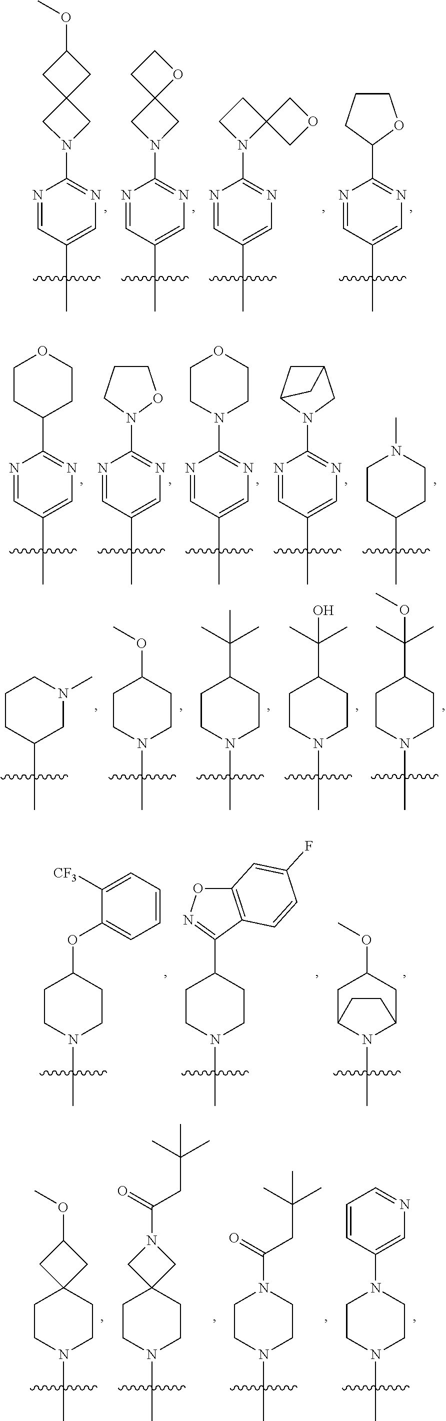

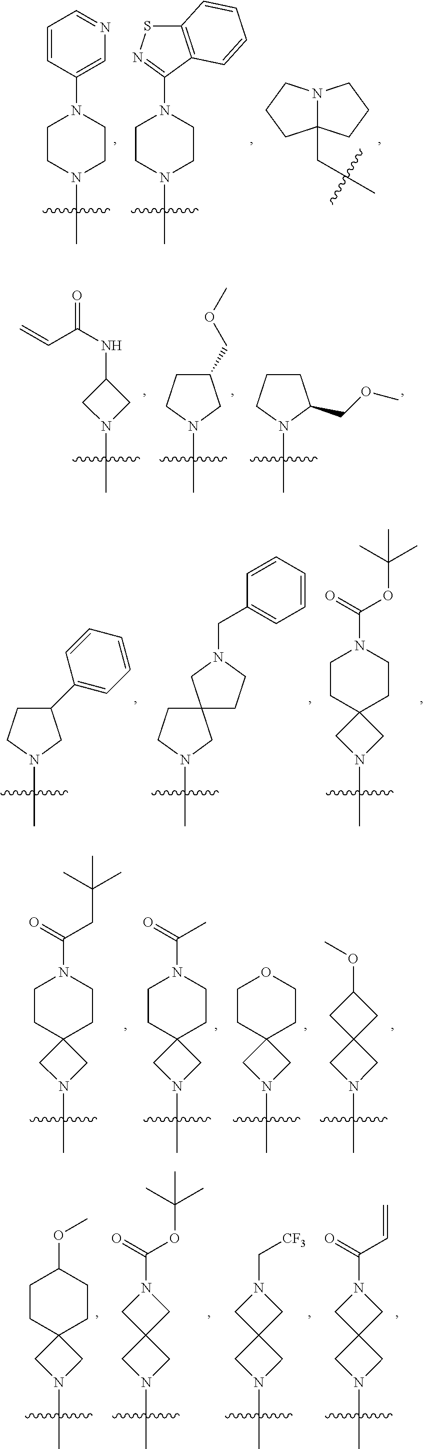

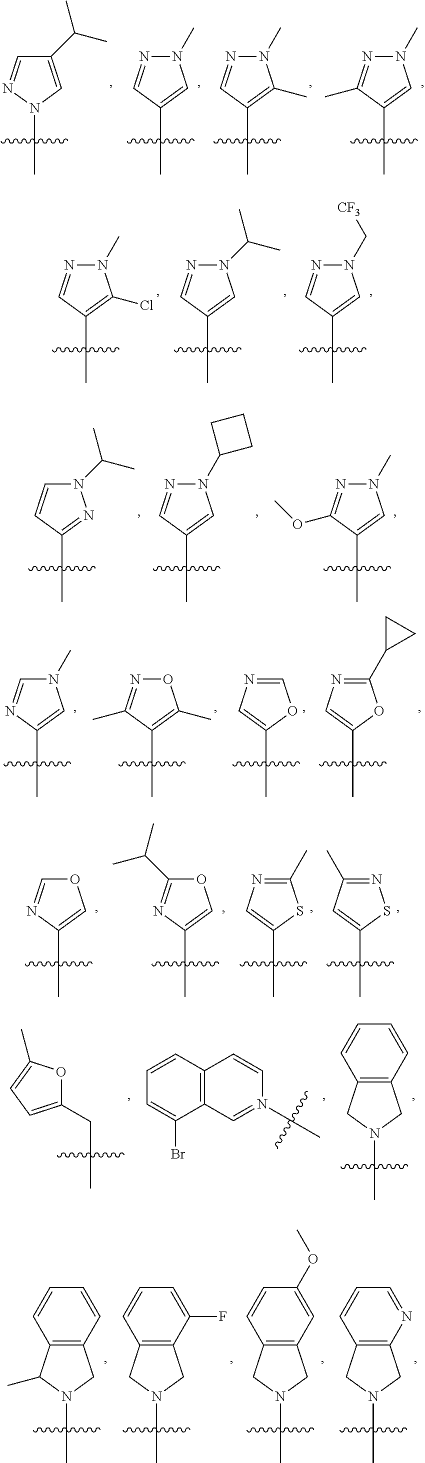

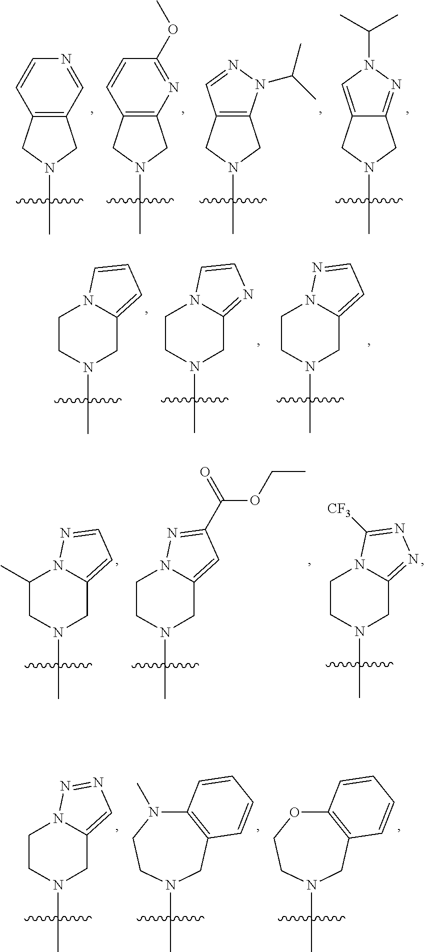

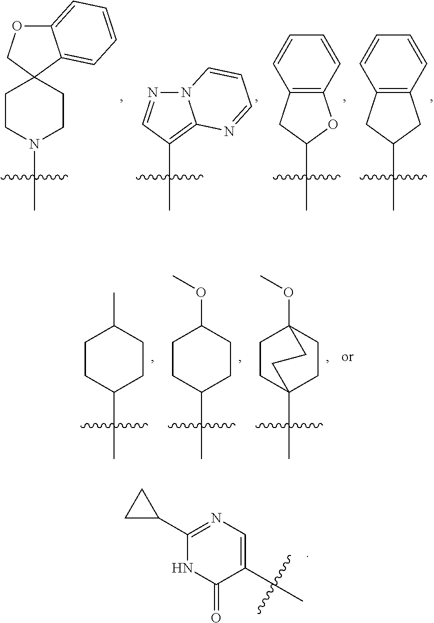

some embodiments, R³ has one of the following structures:

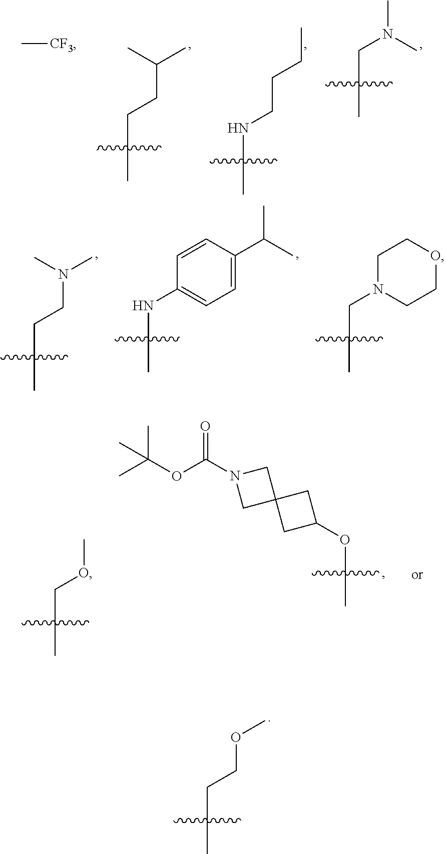

some embodiments, R³ has one of the following structures:

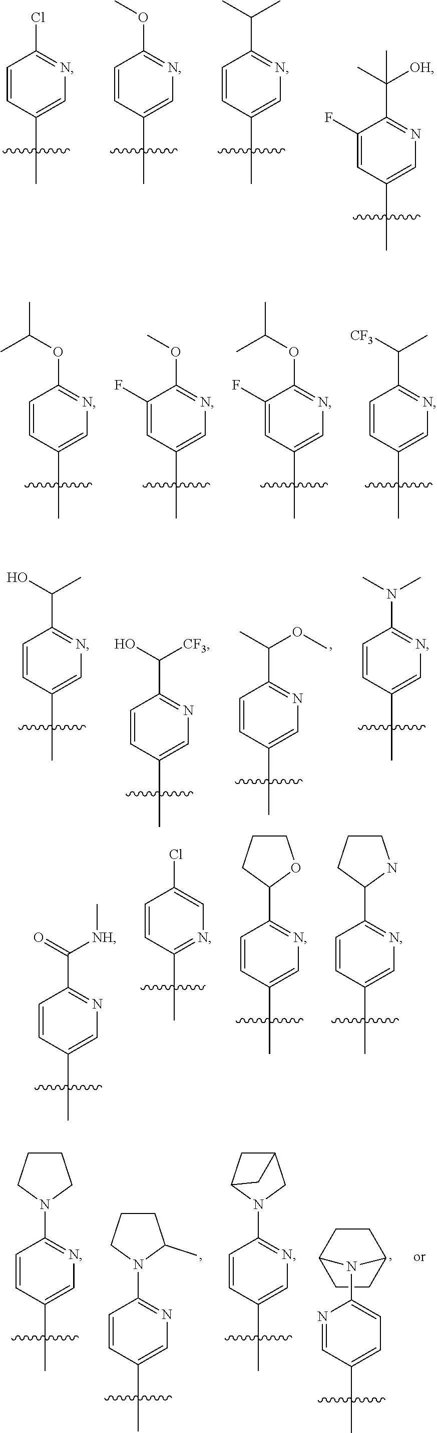

In certain embodiments, R³ has one of the following structures:

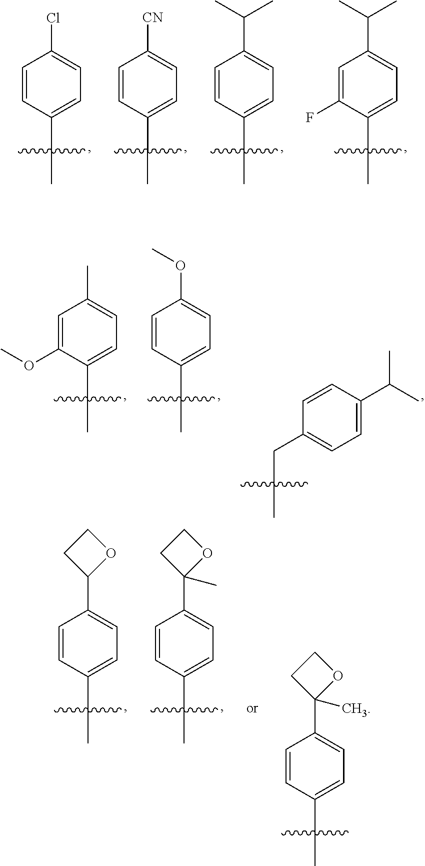

In some embodiments, R³ has one of the following structures:

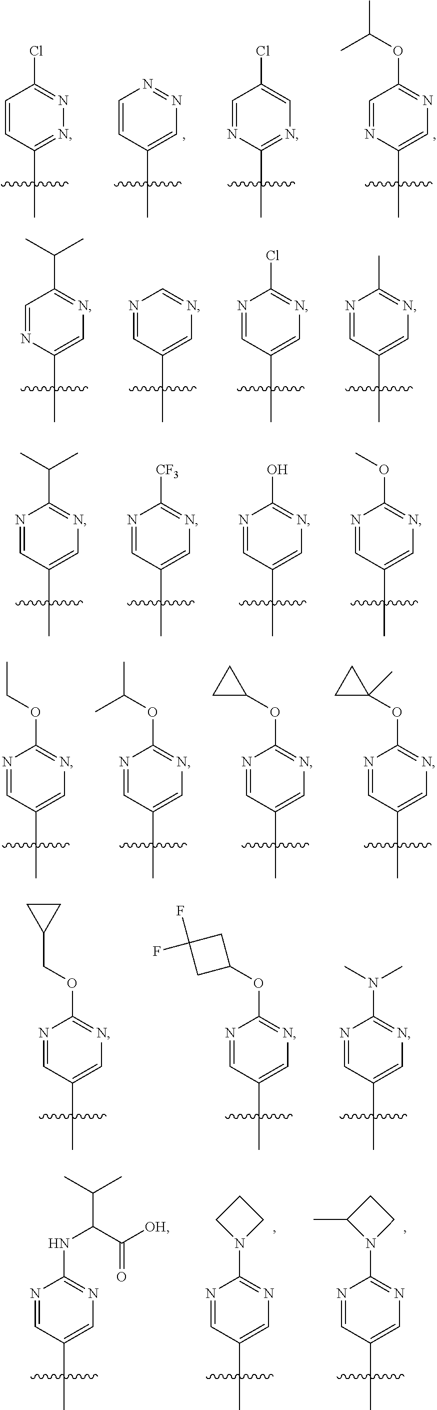

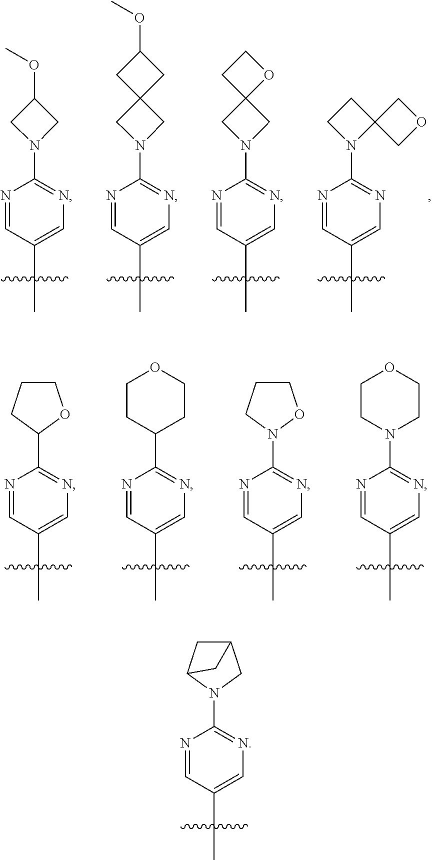

In certain embodiments, R³ has one of the following structures:

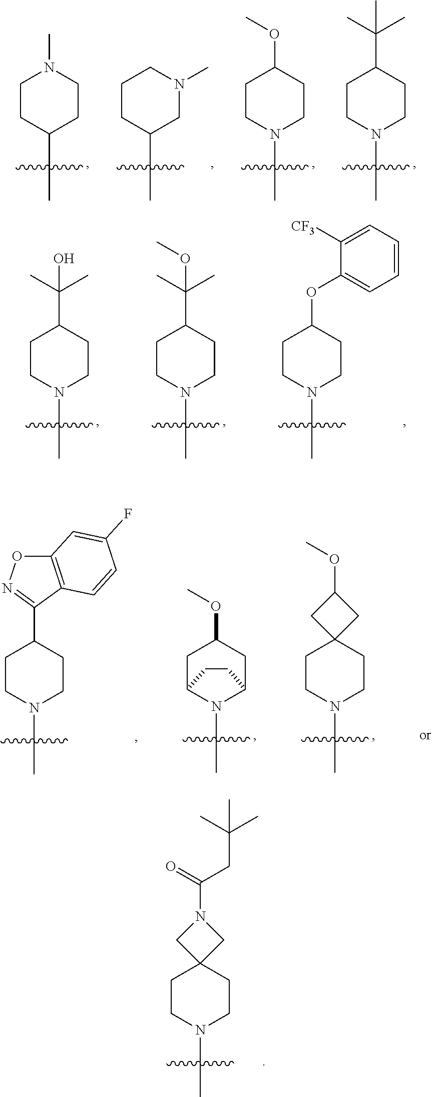

In some embodiments, R³ has one of the following structures:

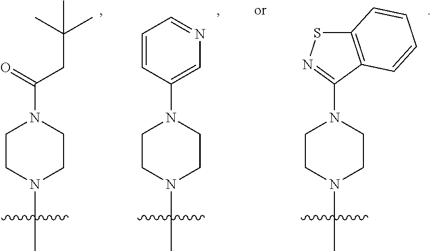 [SMILES: *N1CCN(C(=O)CC(C)(C)C)CC1 ; *N1CCN(c2cccnc2)CC1 ; *N1CCN(c2nsc3ccccc23)CC1]

In some embodiments, R³ has the following structure:

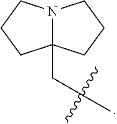 [SMILES: *CC12CCCN1CCC2]

In certain embodiments, R³ has one of the following structures:

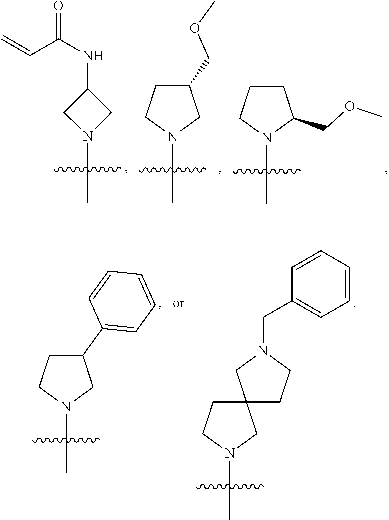

In certain embodiments, R³ has one of the following structures:

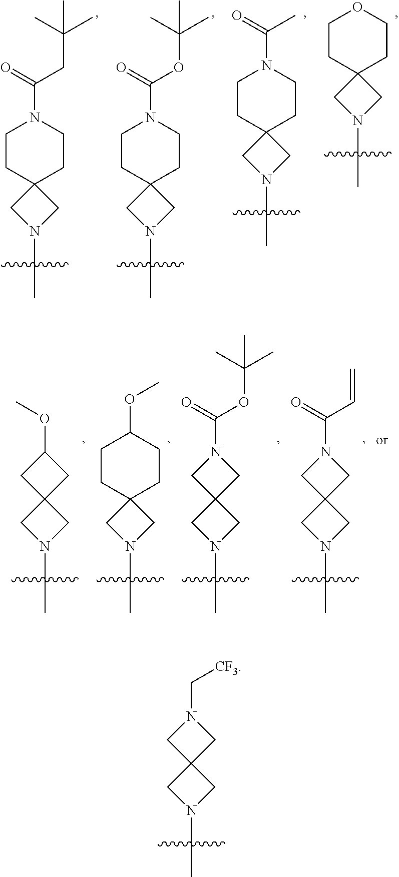

In some embodiments, R³ has one of the following structures: In some embodiments, R³ has one of the following structures:

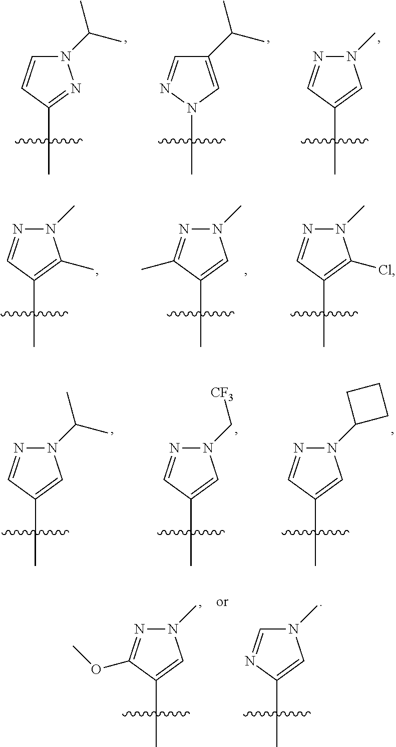

In some embodiments, R³ has the following structure:

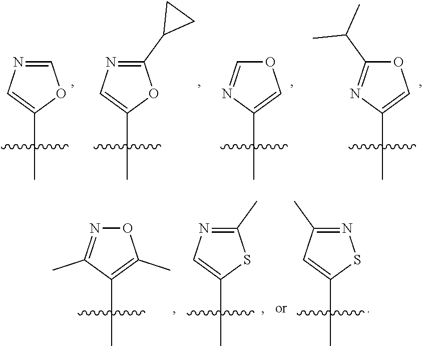

In certain embodiments, R³ has the following structure:

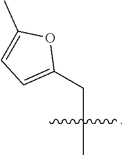 [SMILES: *Cc1ccc(C)o1]

In certain, R³ has the following structure:

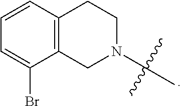 [SMILES: *N1CCc2cccc(Br)c2C1]

In some embodiments, R³ has one of the following structures:

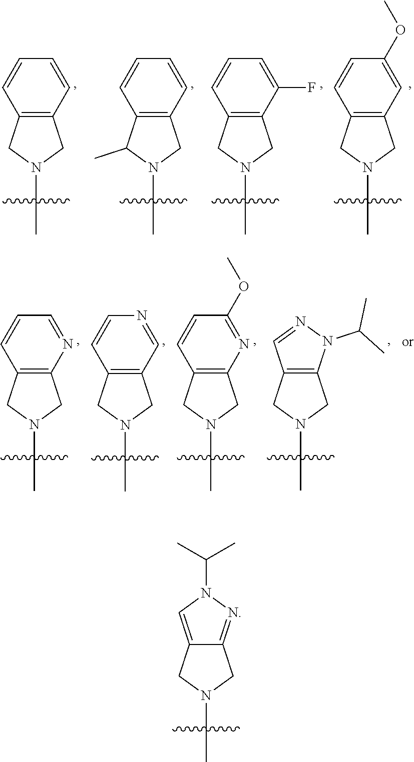

In some embodiments, R³ has one of the following structures:

In certain embodiments, R³ has one of the following structures:

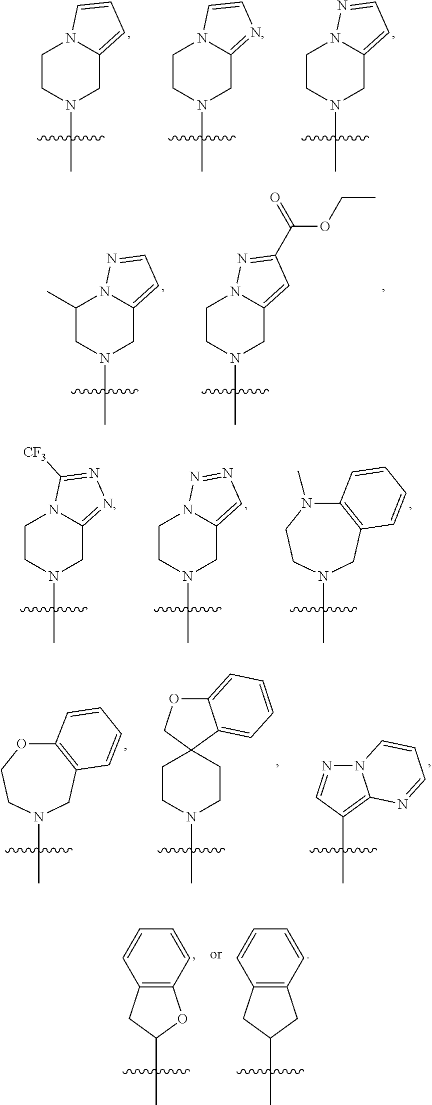

In certain embodiments, R³ has one of the following structures:

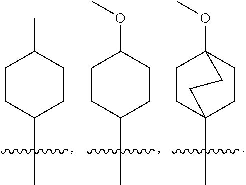

In certain embodiments, R³ has the following structure:

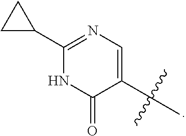 [SMILES: *c1cnc(C2CC2)[nH]c1=O]

In some embodiments, R³ and R¹ together have one of the following structures:

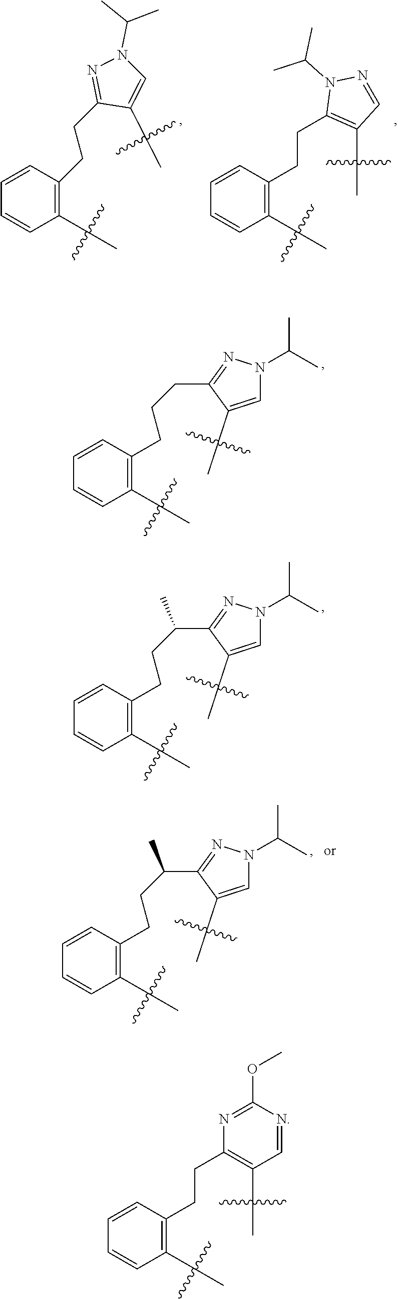 [SMILES: *c1ccccc1CCc1nn(C(C)C)cc1* ; *c1ccccc1CCc1c(*)cnn1C(C)C ; *c1ccccc1CCCc1nn(C(C)C)cc1* ; *c1ccccc1CC[C@H](C)c1nn(C(C)C)cc1* ; *c1ccccc1CC[C@@H](C)c1nn(C(C)C)cc1* ; *c1ccccc1CCc1nc(OC)ncc1*]

In certain embodiments, R³ is alkyl, —R⁸—N(R⁹)₂, —R⁸—OR⁹, or R³ is selected from:

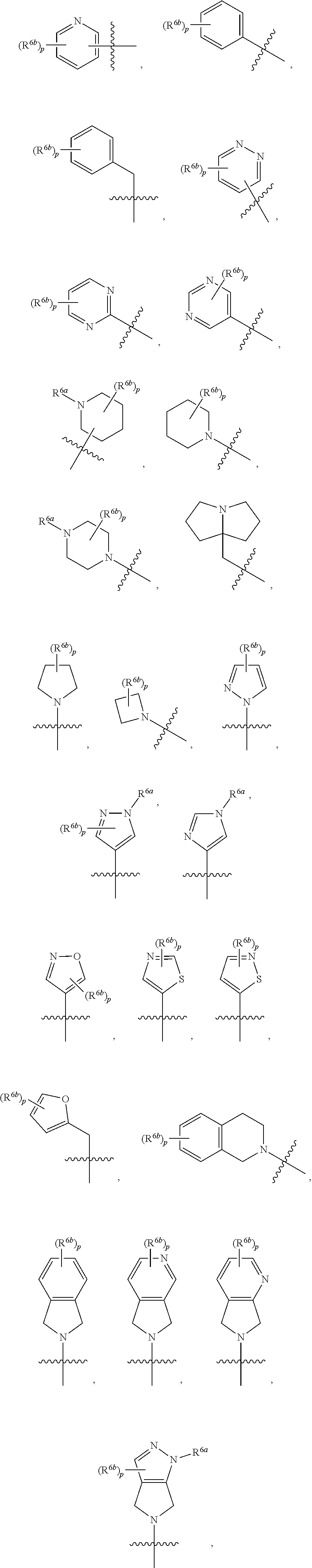

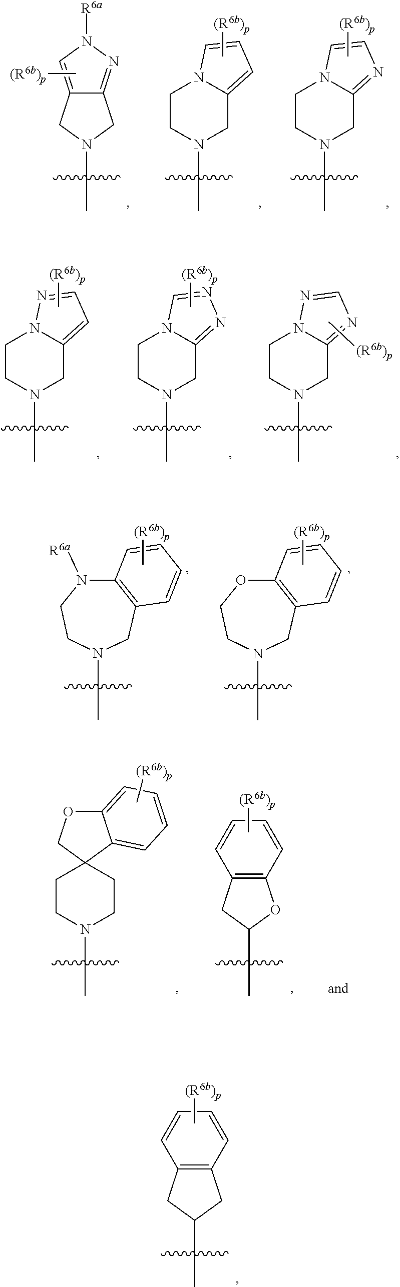

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In certain specific embodiments, R³ is alkyl, —R⁸—N(R⁹)₂, —R⁸—OR⁹, or R³ is selected from:

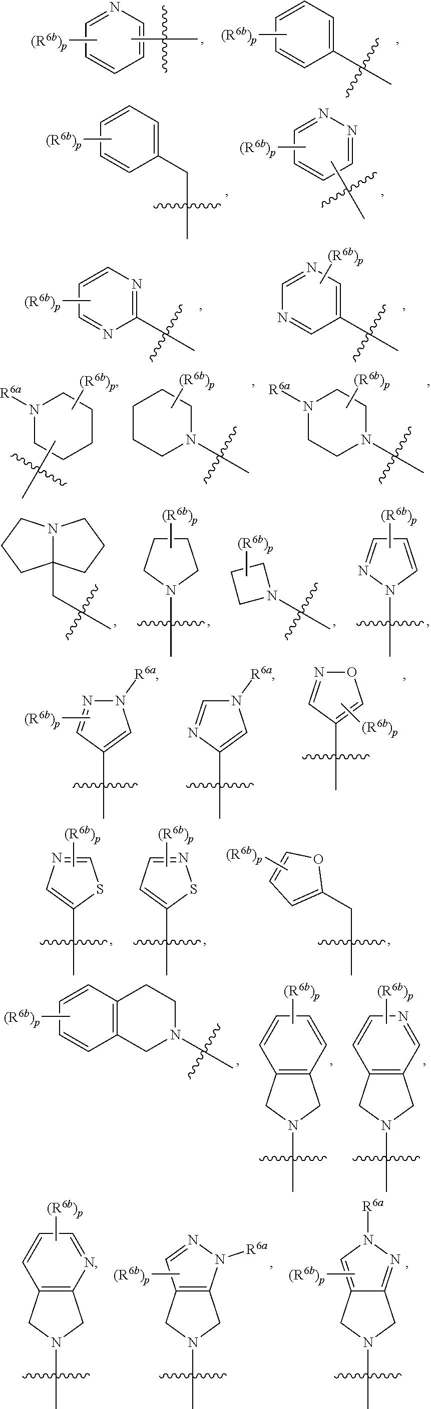

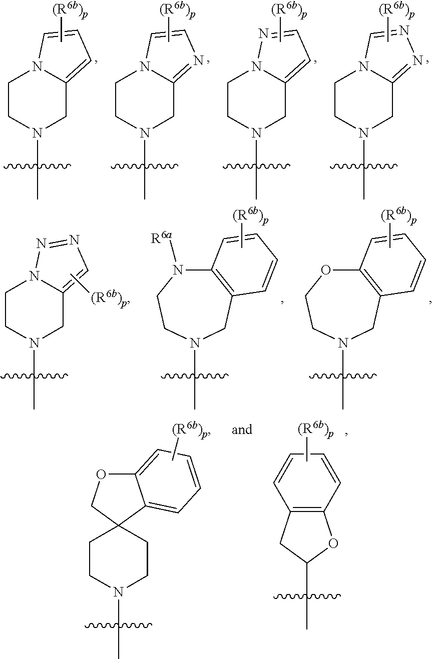

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{8b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In more specific embodiments, R³ is alkyl, —R⁸—N(R⁹)₂, —R⁸—OR⁹, or R³ is selected from:

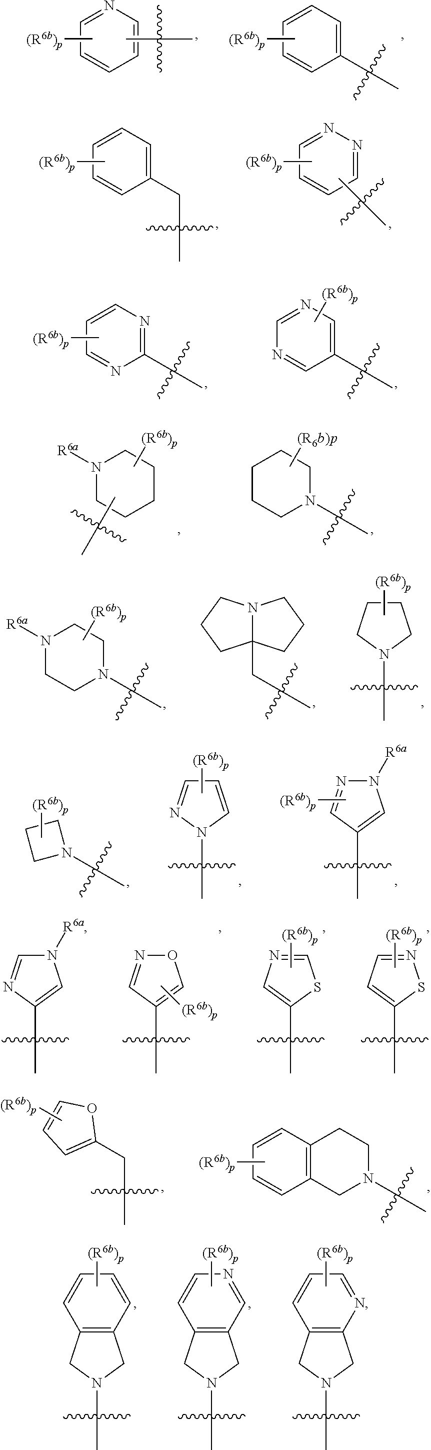

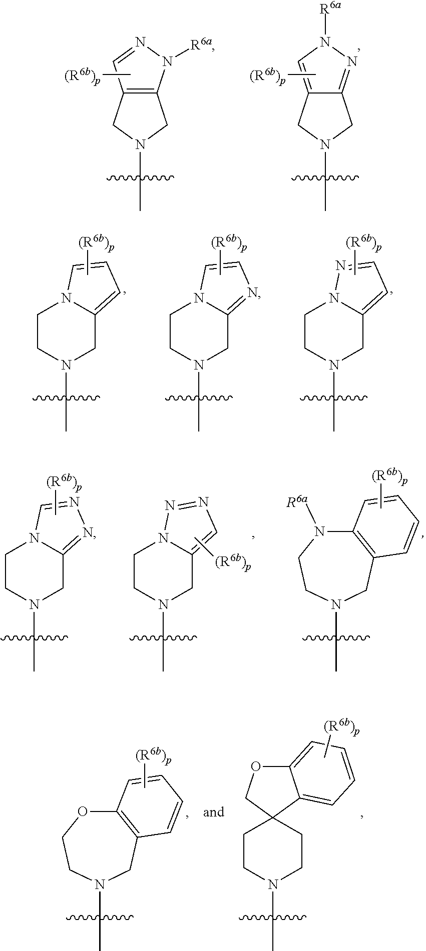

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, —R⁸—OR⁹, or R³ is selected from:

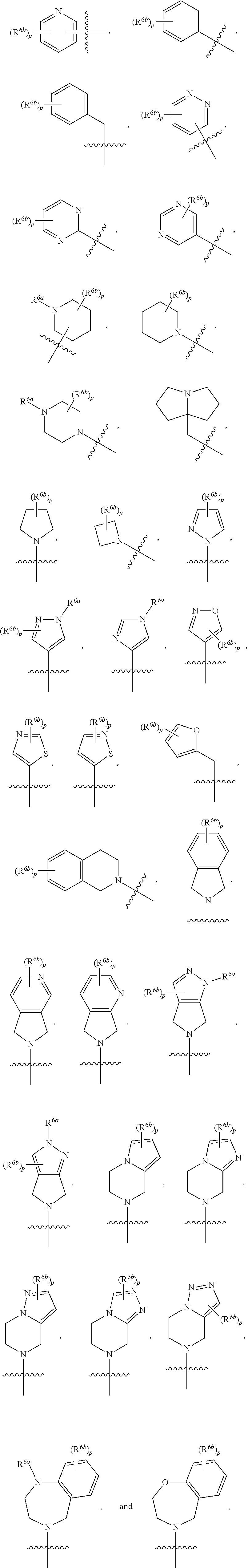

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R⁶¹'s, together with the carbons to which they are attached, form an optionally substituted alkylene chain.

In some embodiments, R³ is alkyl, —R⁸—N(R⁹)₂, —R⁸—OR⁹, or R³ is selected from:

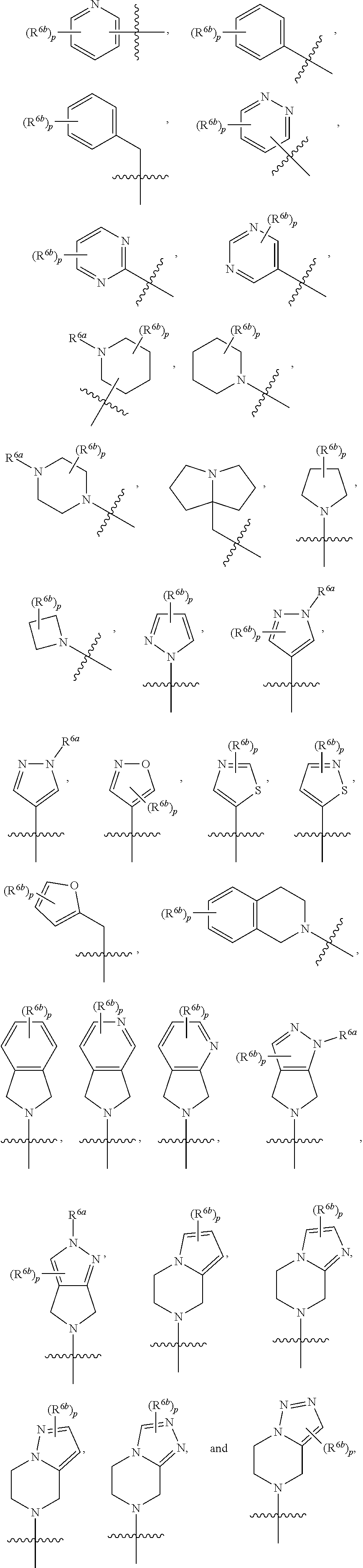

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, —R⁸—OR⁹, or R³ is selected from:

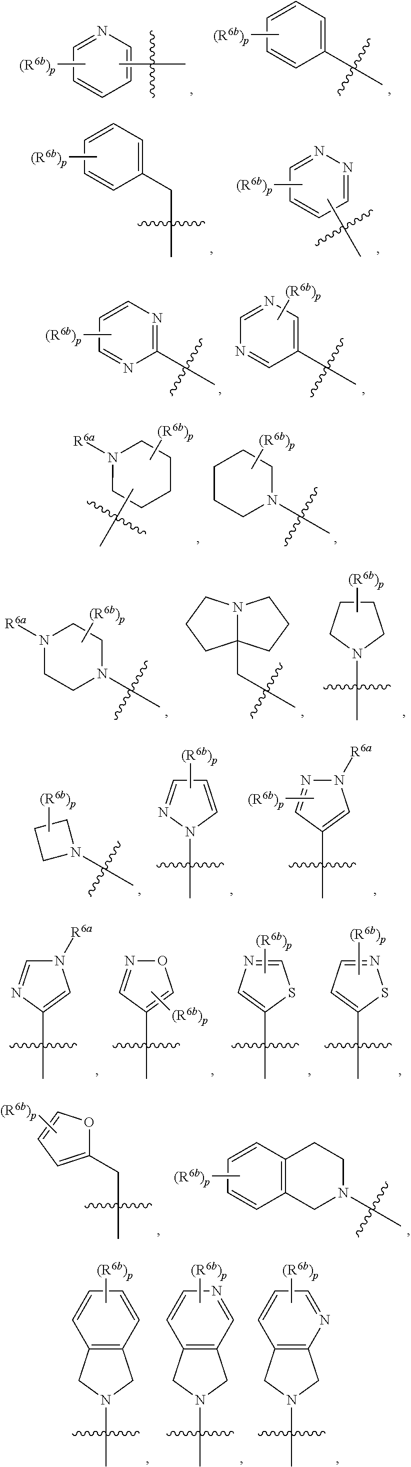

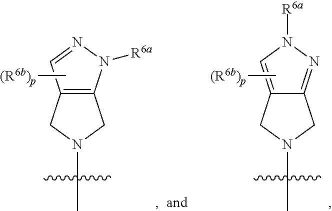

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In certain embodiments, R³ is alkyl, —R⁸—N(R⁹)₂, —R⁸—OR⁹, or R³ is selected from:

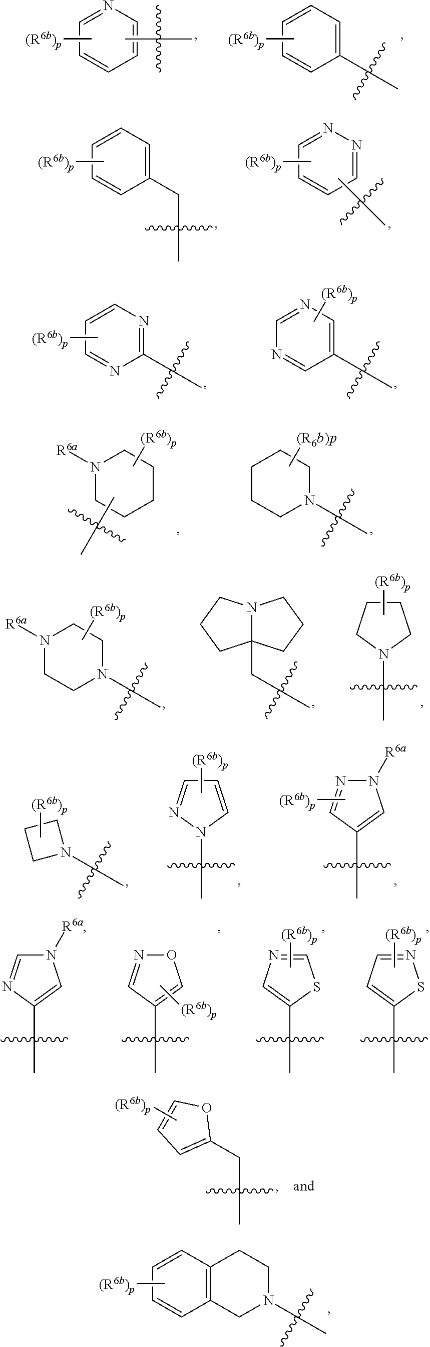

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some specific embodiments, R³ is alkyl, —R⁸—N(R⁹)₂, —R⁸—OR⁹, or R³ is selected from:

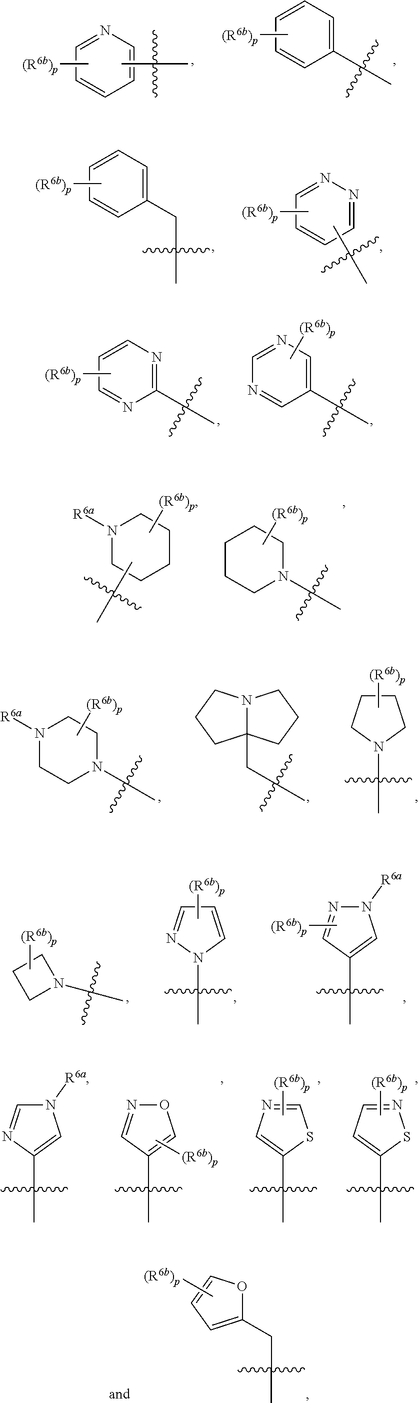

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In certain specific embodiments, R³ is alkyl, —R⁸—N(R⁹)₂, —R⁸—OR⁹, or R³ is selected from:

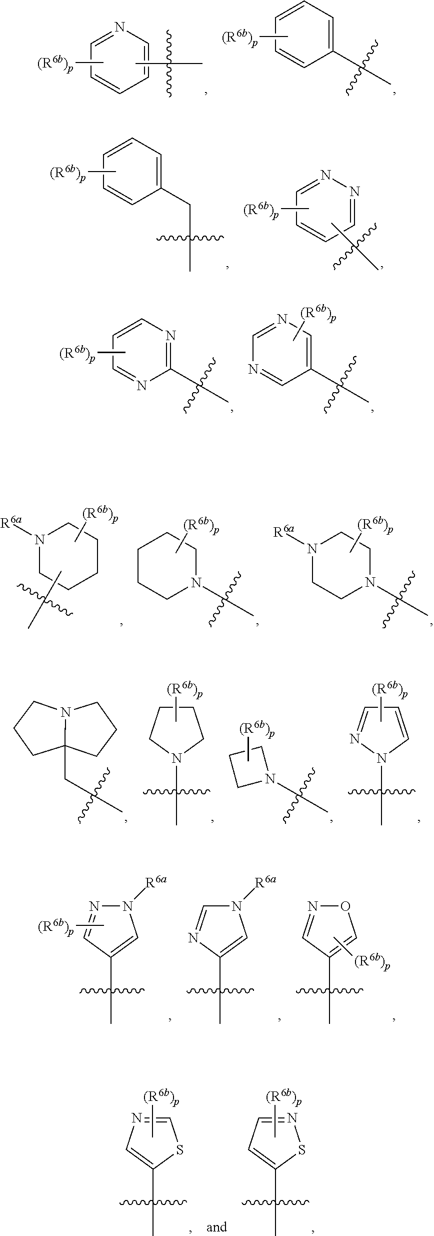

wherein:
p is 0, 1, 2, 3, 4, or 5;
R^{8a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some more specific embodiments, R³ is alkyl, —R⁸—N(R⁹)₂, —R⁸—OR⁹, or R³ is selected from:

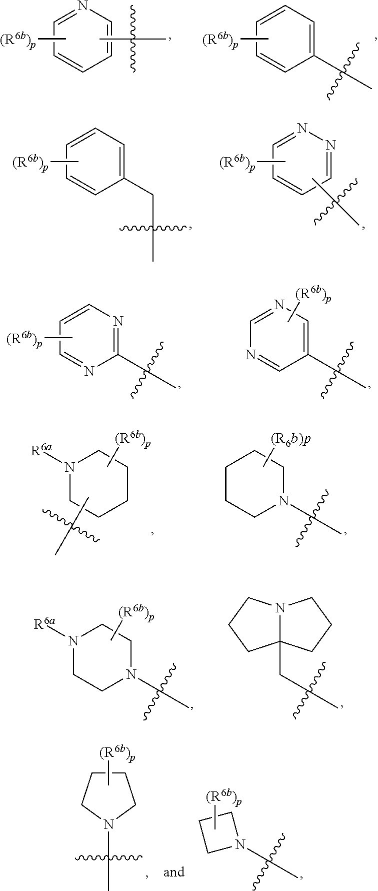

wherein:
p is 0, 1, 2, 3, 4, or 5;
R^{8a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In certain more specific embodiments, R³ is alkyl, —R⁸—N(R⁹)₂, —R⁸—OR⁹, or R³ is selected from:

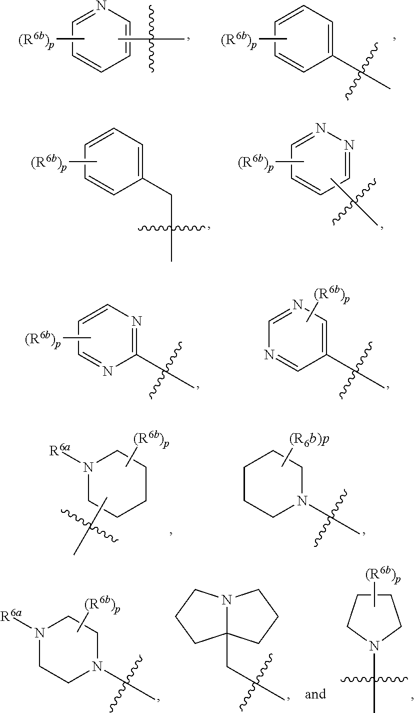

wherein:
p is 0,1,2,3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R⁶¹'s, together with the carbons to which they are attached, form an optionally substituted alkylene chain.

In some other specific embodiments, R³ is alkyl, —R⁸—N(R⁹)₂, —R⁸—OR⁹, or R³ is selected from:

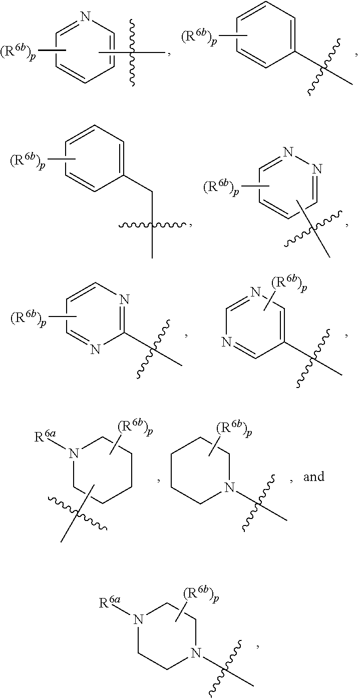

wherein:
p is 0,1,2,3,4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, —R⁸—OR⁹, or R³ is selected from:

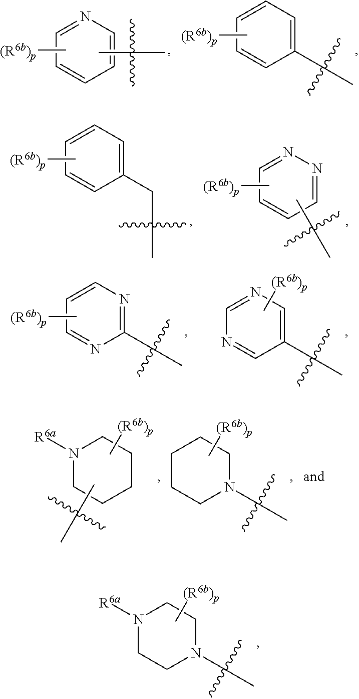

wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, —R⁸—OR⁹, or R³ is selected from:

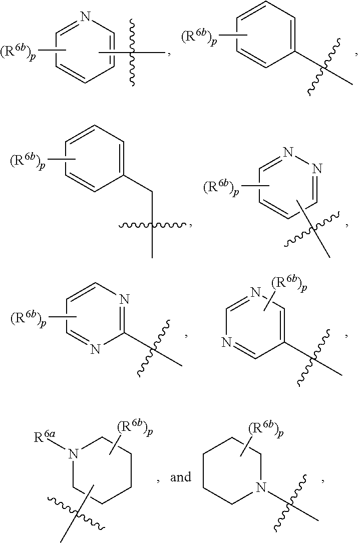

wherein:
p is 0, 1, 2, 3, 4, or 5;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, —R⁸—OR⁹, or R³ is selected from:

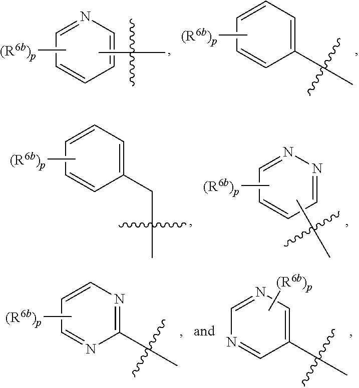

wherein:
p is 0, 1, 2, 3, 4, or 5;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In certain embodiments, R³ is alkyl, —R⁸—N(R⁹)₂, —R⁸—OR⁹, or R³ is selected from:

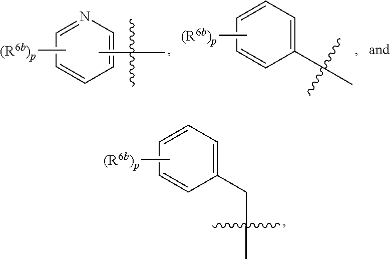

wherein:
p is 0,1,2,3,4, or 5;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some specific embodiments, R³ is alkyl, —R⁸—N(R⁹)₂, —R⁸—OR⁹, or R³ is:

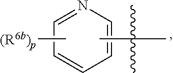

wherein:
p is 0, 1, 2, 3, 4, or 5;
each R^{6b} is independently alkyl, halo, —R⁸—OR⁹, —R⁸—N(R⁹)₂, —R⁸—C(═O)OR⁹, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s, together with the carbons to which they are attached, form an optionally substituted alkylene chain.

In some embodiments, R³ is alkyl, —R⁸—N(R⁹)₂, or —R⁸—OR⁹. In certain embodiments, R³ is alkyl or —R⁸—N(R⁹)₂. In some specific embodiments, R³ is alkyl. In some more specific embodiments, R³ has one of the following structures:

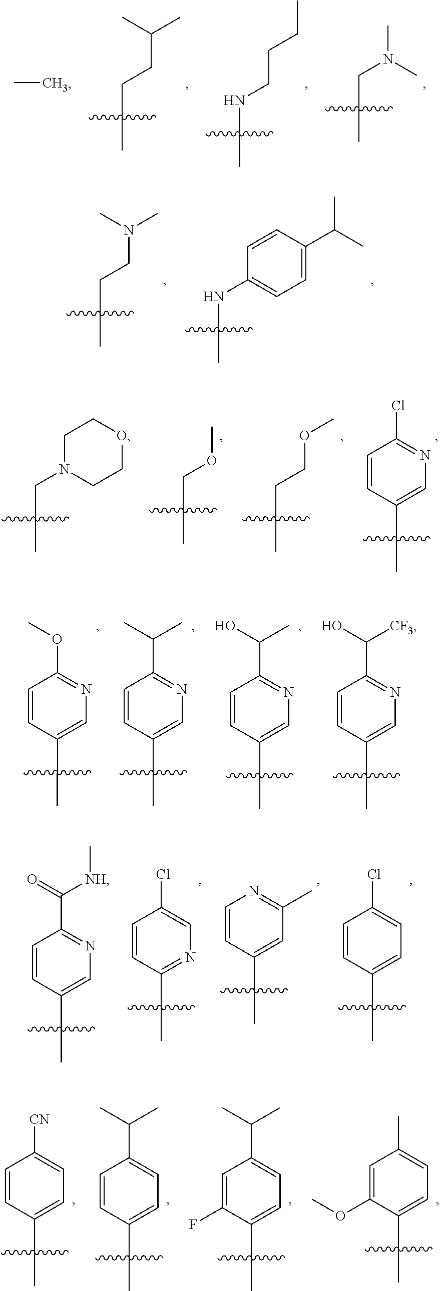

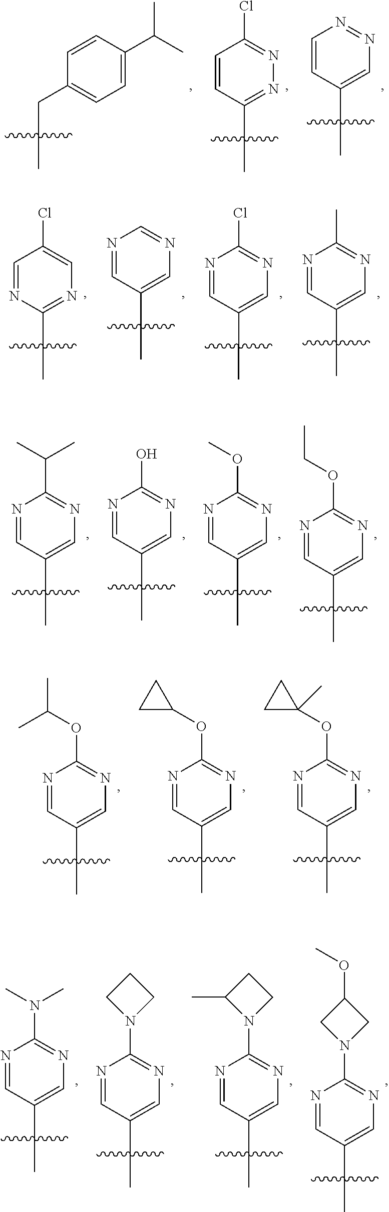

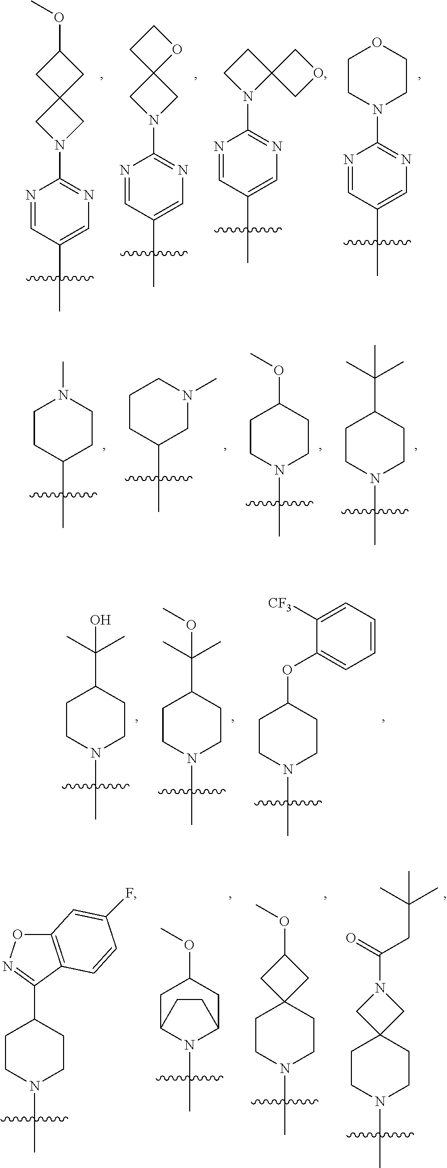

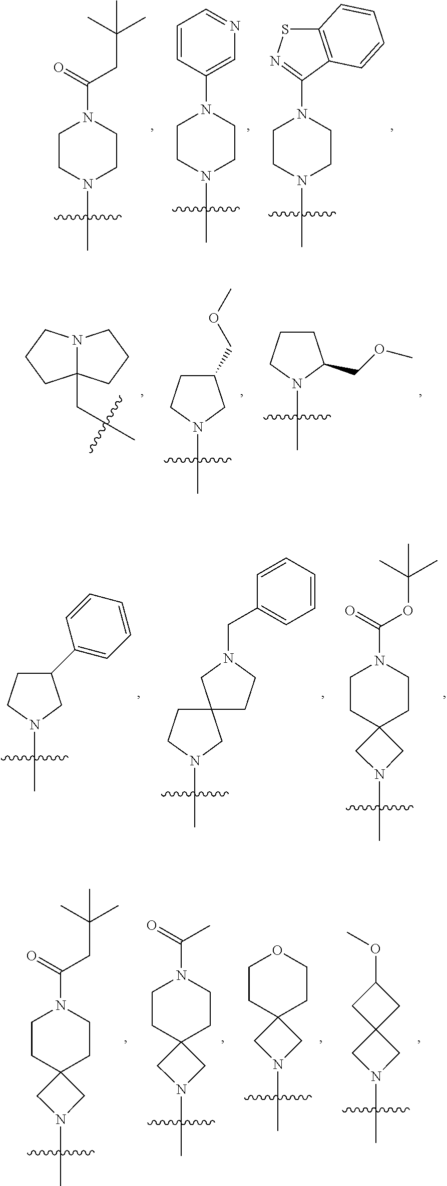

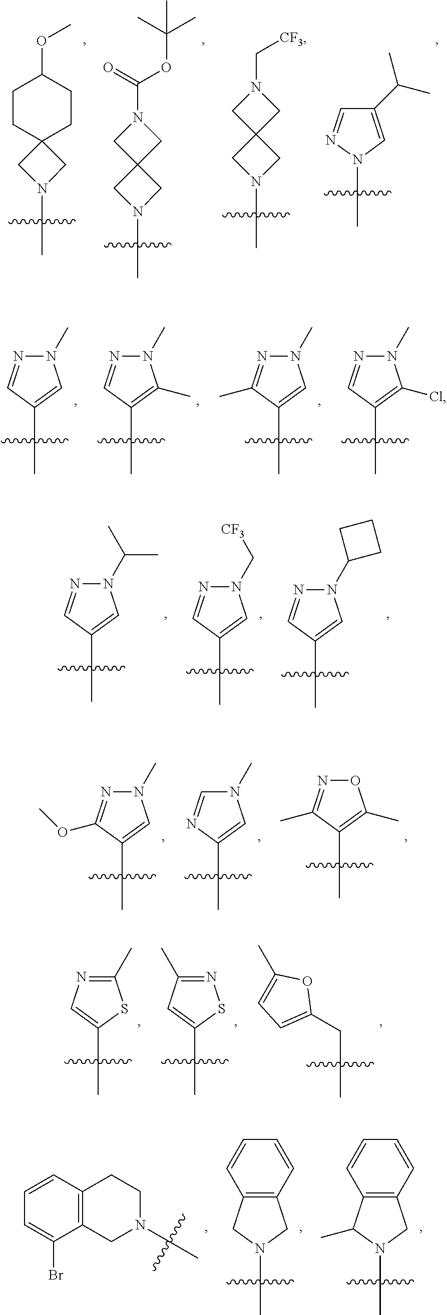

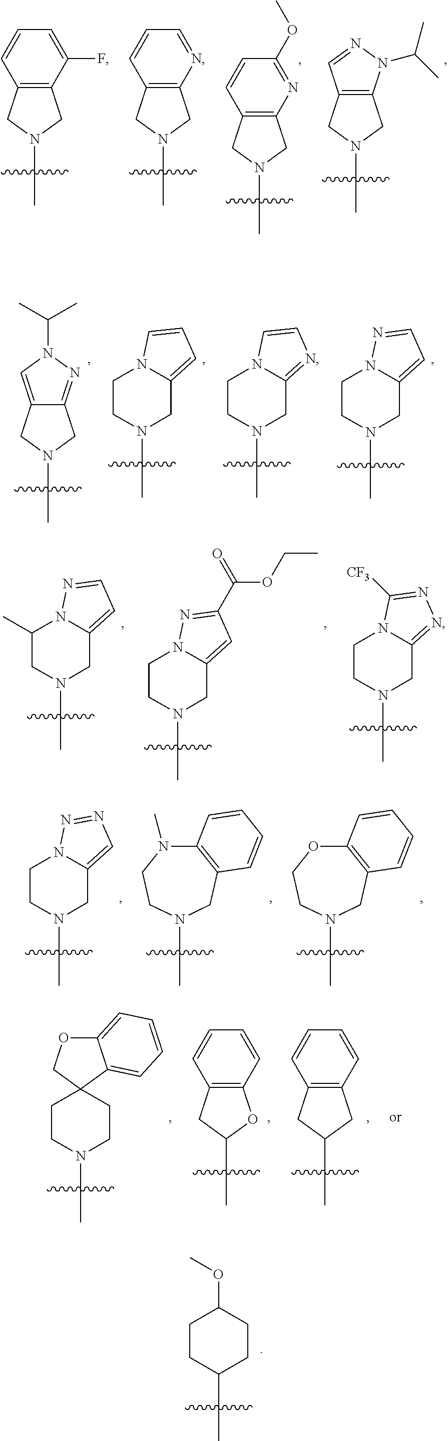

In some embodiments, R³ has one of the following structures:

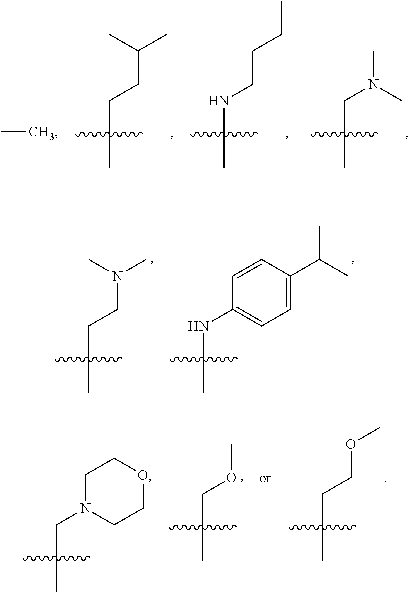

In some embodiments, R³ has one of the following structures:

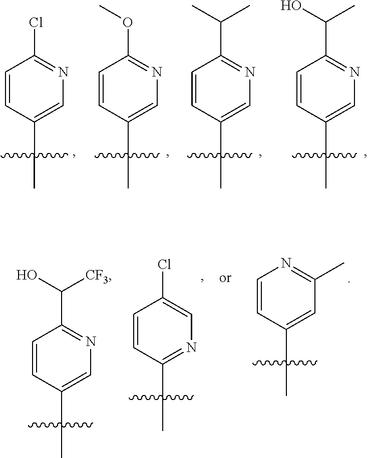

In certain embodiments, R³ has one of the following structures:

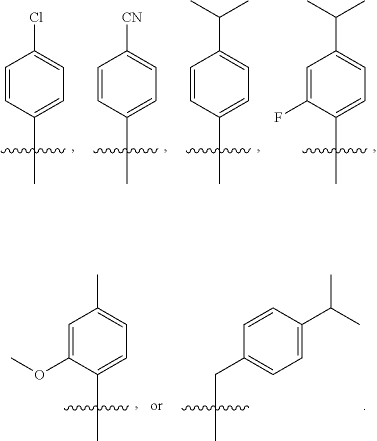

In some specific embodiments, R³ has one of the following structures:

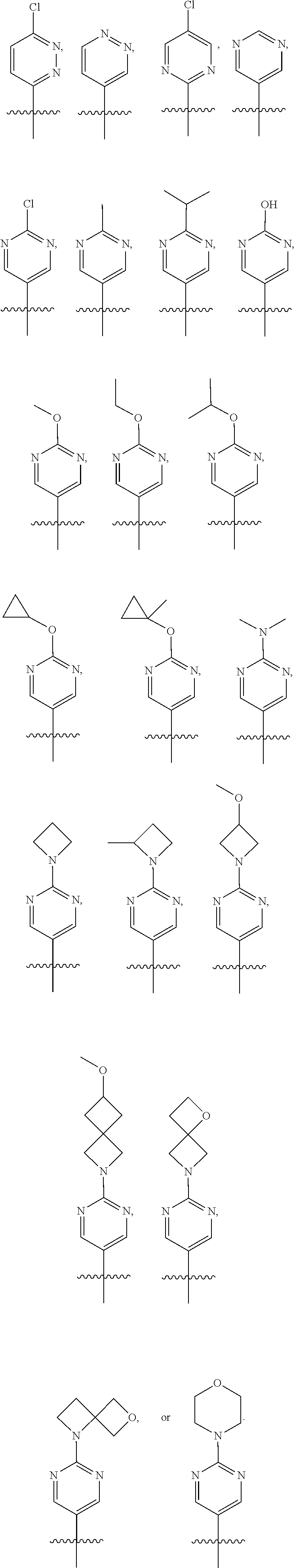

In some embodiments, R³ has one of the following structures:

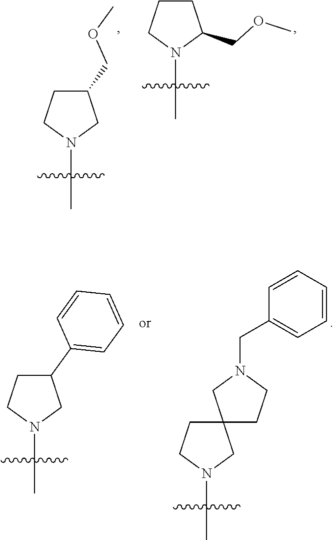

In certain specific embodiments, R³ has one of the following structures:

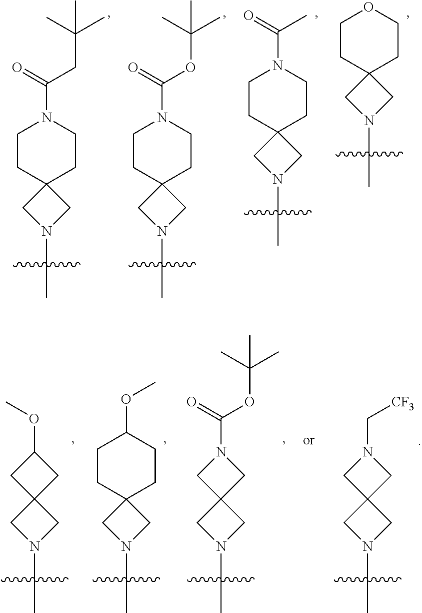

In more specific embodiments, R³ has one of the following structures:

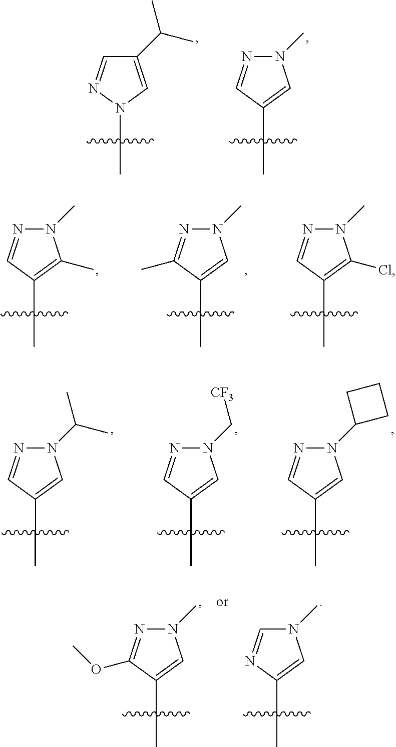

In certain embodiments, R³ has one of the following structures:

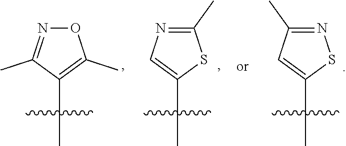

In certain embodiments, R³ has one of the following structures:

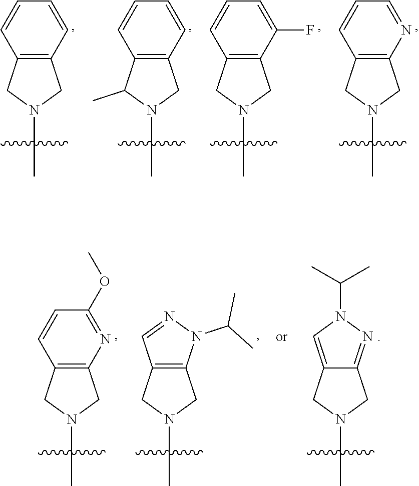

In some specific embodiments, R³ has the following structure:

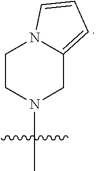 [SMILES: *N1CCn2cccc2C1]

In certain specific embodiments, R³ has the following structure:

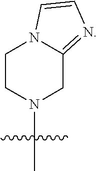 [SMILES: *N1CCn2ccnc2C1]

In some more specific embodiments, R³ has one of the following structures:

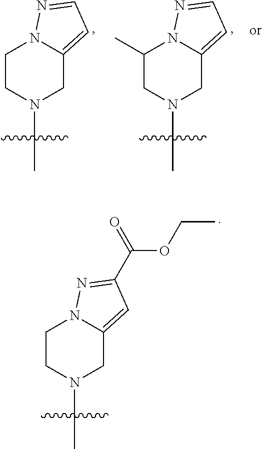

In certain specific embodiments, R³ has the following structure:

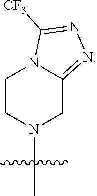 [SMILES: *N1CCn2c(nnc2C(F)(F)F)C1]

In some embodiments, R³ has the following structure:

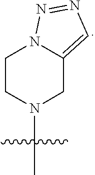 [SMILES: *N1CCn2nncc2C1]

In certain embodiments, R³ has the following structure:

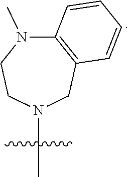 [SMILES: *N1CCN(C)c2ccccc2C1]

In some specific embodiments, R³ has the following structure:

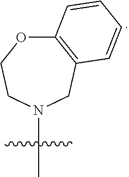 [SMILES: *N1CCOc2ccccc2C1]

In certain specific embodiments, R³ has the following structure:

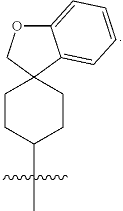 [SMILES: *C1CCC2(CC1)COc1ccccc12]

In some embodiments, R³ has the following structure:

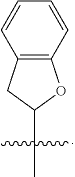 [SMILES: *C1Cc2ccccc2O1]

In certain embodiments, R³ has the following structure:

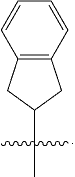 [SMILES: *C1Cc2ccccc2C1]

In some embodiments, R³ has one of the following structures:

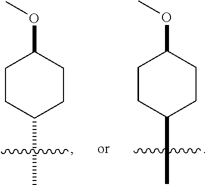

In certain embodiments, R^{3a} is hydrogen. In some specific embodiments, R^{3a} is alkyl. In some more specific embodiments, R³² is methyl.

In some embodiments, R⁴ is hydrogen. In certain embodiments, R⁴ is alkyl. In some specific embodiments, R⁴ is —CH₃. In certain specific embodiments, R⁴ is halo. In some more specific embodiments, R⁴ is fluoro. In certain specific embodiments, R⁴ is halo. In some more specific embodiments, R⁴ is chloro. In certain specific embodiments, R⁴ is halo. In some more specific embodiments, R⁴ is fluoro or chloro. In some embodiments, R⁴ is —R⁸—OR⁹. In more specific embodiments, R⁴ is —OH or —OCH₃. In more specific embodiments, R⁴ is —OH. In more specific embodiments, R⁴ is —OCH₃. In some embodiments, R⁴ is haloalkyl. In more specific embodiments, R⁴ is —CF₃. In certain embodiments, R⁴ is cyano.

In some specific embodiments, R⁷ is alkyl. In certain embodiments, R⁷ is —CH₃.

In some embodiments, the compound is a compound as set forth in Table 1 below as a stereoisomer, enantiomer, or tautomer thereof or mixtures thereof; or a pharmaceutically acceptable salt, solvate, or prodrug thereof.

TABLE 1
Representative compounds of formula (I) or (II)

| Compound | | |
|---|---|---|
| Number | Compound Structure | Compound Name |
| 1 | 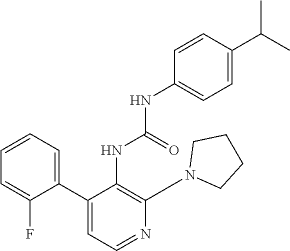 [SMILES: CC(C)c1ccc(NC(=O)Nc2c(-c3ccccc3F)ccnc2N2CCCC2)cc1] | 1-(4-(2-fluorophenyl)-2- (pyrrolidin-1-yl)pyridin- 3-yl)-3-(4-isopropylphen- yl)urea |
| 2 | 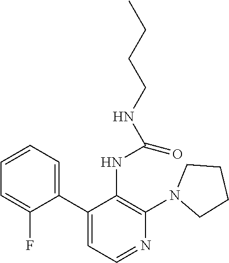 [SMILES: CCCCNC(=O)Nc1c(-c2ccccc2F)ccnc1N1CCCC1] | 1-butyl-3-(4-(2-fluoro- phenyl)-2-(pyrrolidin-1- yl)pyridin-3-yl)urea |
| 3 | 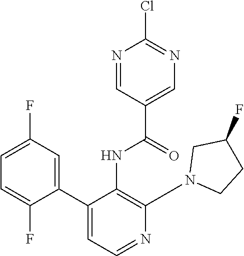 [SMILES: O=C(Nc1c(-c2cc(F)ccc2F)ccnc1N1CC[C@H](F)C1)c1cnc(Cl)nc1] | (S)-2-chloro-N-(4-(2,5- difluorophenyl)-2-(3- fluoropyrrolidin-1-yl)- pyridin-3-yl)pyrimidine- 5-carboxamide |
| 4 | 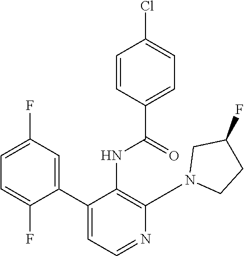 [SMILES: O=C(Nc1c(-c2cc(F)ccc2F)ccnc1N1CC[C@H](F)C1)c1ccc(Cl)cc1] | (S)-4-chloro-N-(4-(2,5- difluorophenyl)-2-(3- fluoropyrrolidin-1-yl)- pyridin-3-yl)benzamide |
| 5 | 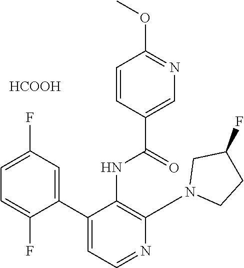 [SMILES: "HCOOH" ; COc1ccc(C(=O)Nc2c(-c3cc(F)ccc3F)ccnc2N2CC[C@H](F)C2)cn1] | (S)-N-(4-(2,5-difluoro- phenyl)-2-(3-fluoropyr- rolidin-1-yl)pyridin-3- yl)-6-methoxynicotin- amide |
| 6 | 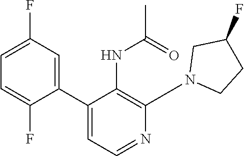 [SMILES: CC(=O)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)acetamide |
| 7 | 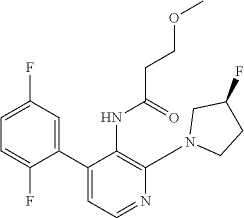 [SMILES: COCCC(=O)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)-3-methoxypropan- amide |
| 8 | 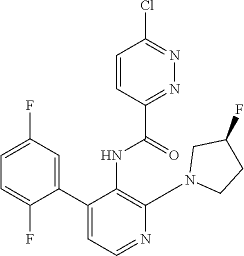 [SMILES: O=C(Nc1c(-c2cc(F)ccc2F)ccnc1N1CC[C@H](F)C1)c1ccc(Cl)nn1] | (S)-6-chloro-N-(4-(2,5- difluorophenyl)-2-(3- fluoropyrrolidin-1-yl)- pyridin-3-yl)pyridazine- 3-carboxamide |
| 9 | 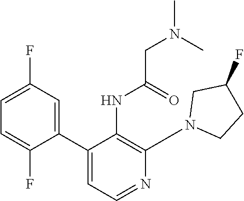 [SMILES: CN(C)CC(=O)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)-2-(dimethylamino)- acetamide |
| 10 | 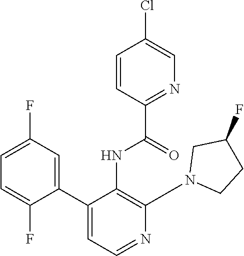 [SMILES: O=C(Nc1c(-c2cc(F)ccc2F)ccnc1N1CC[C@H](F)C1)c1ccc(Cl)cn1] | (S)-5-chloro-N-(4-(2,5- difluorophenyl)-2-(3- fluoropyrrolidin-1-yl)pyr- idin-3-yl)picolinamide |
| 11 | 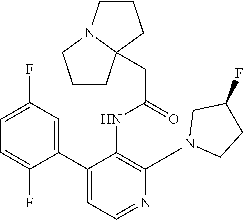 [SMILES: O=C(CC12CCCN1CCC2)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)-2-(tetrahydro-1H- pyrrolizin-7a(5H)-yl)- acetamide |
| 12 | 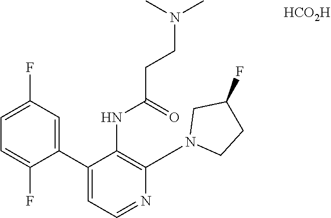 [SMILES: CN(C)CCC(=O)Nc1c(-c2cc(F)ccc2F)ccnc1N1CC[C@H](F)C1 ; "HCO2H"] | (S)-N-(4-(2,5-difluoro- phenyl)-2-(3-fluoropyr- rolidin-1-yl)pyridin-3- yl)-3-(dimethylamino)- propanamide |
| 13 | 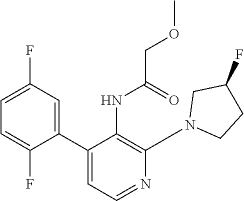 [SMILES: COCC(=O)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)-2-methoxyacetamide |
| 14 | 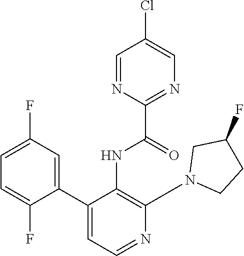 [SMILES: O=C(Nc1c(-c2cc(F)ccc2F)ccnc1N1CC[C@H](F)C1)c1ncc(Cl)cn1] | (S)-5-chloro-N-(4-(2,5- difluorophenyl)-2-(3- fluoropyrrolidin-1-yl)- pyridin-3-yl)pyrimidine- 2-carboxamide |
| 15 | 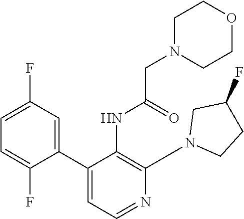 [SMILES: O=C(CN1CCOCC1)Nc1c(-c2cc(F)ccc2F)ccnc1N1CC[C@H](F)C1] | (S)-N-(4-(2,5-difluoro- phenyl)-2-(3-fluoropyr- rolidin-1-yl)pyridin-3- yl)-2-morpholinoacet- amide |
| 16 | 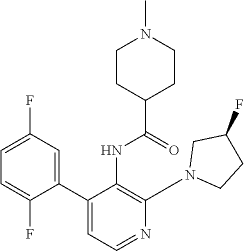 [SMILES: CN1CCC(C(=O)Nc2c(-c3cc(F)ccc3F)ccnc2N2CC[C@H](F)C2)CC1] | (S)-N-(4-(2,5-difluoro- phenyl)-2-(3-fluoropyr- rolidin-1-yl)pyridin-3- yl)-1-methylpiperidine- 4-carboxamide |
| 17 | 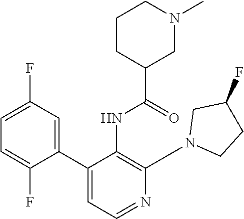 [SMILES: CN1CCCC(C(=O)Nc2c(-c3cc(F)ccc3F)ccnc2N2CC[C@H](F)C2)C1] | N-(4-(2,5-difluorophen- yl)-2-((S)-3-fluoropyr- rolidin-1-yl)pyridin-3- yl)-1-methylpiperidine- 3-carboxamide |
| 18 | 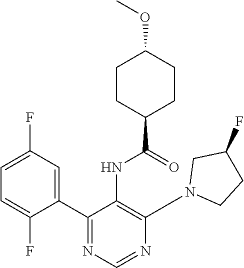 [SMILES: CO[C@H]1CC[C@H](C(=O)Nc2c(-c3cc(F)ccc3F)ncnc2N2CC[C@H](F)C2)CC1] | (1r,4S)-N-(4-(2,5-di- fluorophenyl)-6-((S)-3- fluoropyrrolidin-1-yl)- pyrimidin-5-yl)-4-meth- oxycyclohexane-1- carboxamide |
| 19 | 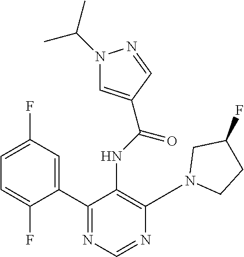 [SMILES: CC(C)n1cc(C(=O)Nc2c(-c3cc(F)ccc3F)ncnc2N2CC[C@H](F)C2)cn1] | N-[4-(2,5-difluorophen- yl)-6-[rac-(3S)-3-fluoro- pyrrolidin-1-yl]pyrim- idin-5-yl]-1-isopropyl- pyrazole-4-carboxamide |
| 20 | 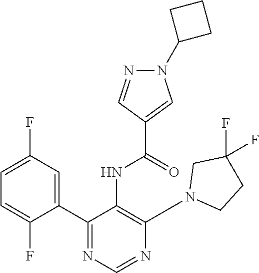 [SMILES: O=C(Nc1c(-c2cc(F)ccc2F)ncnc1N1CCC(F)(F)C1)c1cnn(C2CCC2)c1] | 1-cyclobutyl-N-(4-(2,5- difluorophenyl)-6-(3,3- difluoropyrrolidin-1- yl)pyrimidin-5-yl)-1H- pyrazole-4-carboxamide |
| 21 | 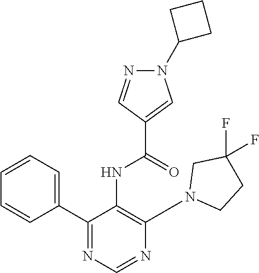 [SMILES: O=C(Nc1c(-c2ccccc2)ncnc1N1CCC(F)(F)C1)c1cnn(C2CCC2)c1] | 1-cyclobutyl-N-(4-(3,3- difluoropyrrolidin-1-yl)- 6-phenylpyrimidin-5-yl)- 1H-pyrazole-4-carbox- amide |
| 22 | 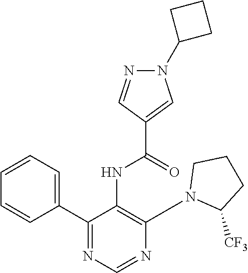 [SMILES: O=C(Nc1c(-c2ccccc2)ncnc1N1CCC[C@@H]1C(F)(F)F)c1cnn(C2CCC2)c1] | (R)-1-cyclobutyl-N-(4- phenyl-6-(2-(trifluoro- methyl)pyrrolidin-1-yl)- pyrimidin-5-yl)-1H- pyrazole-4-carboxamide |
| 23 | 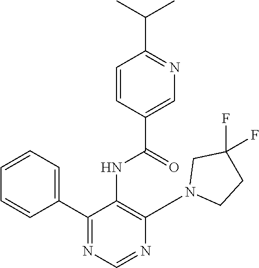 [SMILES: CC(C)c1ccc(C(=O)Nc2c(-c3ccccc3)ncnc2N2CCC(F)(F)C2)cn1] | N-(4-(3,3-difluoropyr- rolidin-1-yl)-6-phenyl- pyrimidin-5-yl)-6-iso- propylnicotinamide |
| 24 | 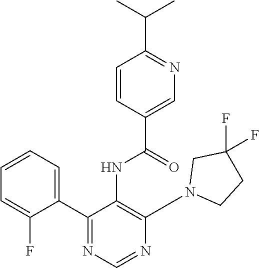 [SMILES: CC(C)c1ccc(C(=O)Nc2c(-c3ccccc3F)ncnc2N2CCC(F)(F)C2)cn1] | N-(4-(3,3-difluoropyr- rolidin-1-yl)-6-(2-fluoro- phenyl)pyrimidin-5-yl)- 6-isopropylnicotinamide |
| 25 | 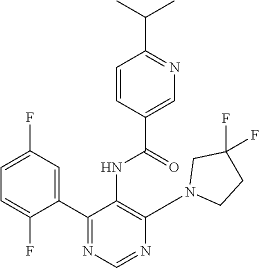 [SMILES: CC(C)c1ccc(C(=O)Nc2c(-c3cc(F)ccc3F)ncnc2N2CCC(F)(F)C2)cn1] | N-(4-(2,5-difluorophen- yl)-6-(3,3-difluoropyr- rolidin-1-yl)pyrimidin-5- yl)-6-isopropylnicotin- amide |
| 26 | 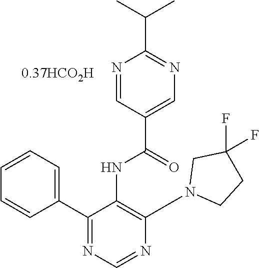 [SMILES: CC(C)c1ncc(C(=O)Nc2c(-c3ccccc3)ncnc2N2CCC(F)(F)C2)cn1] | N-(4-(3,3-difluoropyr- rolidin-1-yl)-6-phenyl- pyrimidin-5-yl)-2-iso- propylpyrimidine-5- carboxamide |
| 27 | 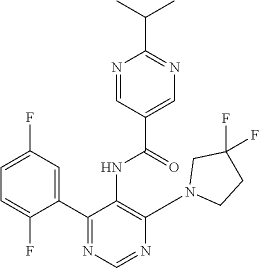 [SMILES: CC(C)c1ncc(C(=O)Nc2c(-c3cc(F)ccc3F)ncnc2N2CCC(F)(F)C2)cn1] | N-(4-(2,5-difluorophen- yl)-6-(3,3-difluoropyr- rolidin-1-yl)pyrimidin-5- yl)-2-isopropylpyrim- idine-5-carboxamide |
| 28 | 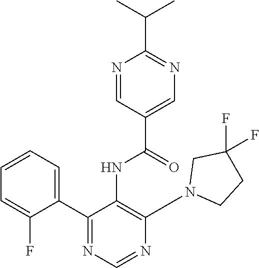 [SMILES: CC(C)c1ncc(C(=O)Nc2c(-c3ccccc3F)ncnc2N2CCC(F)(F)C2)cn1] | N-(4-(3,3-difluoropyr- rolidin-1-yl)-6-(2-fluoro- phenyl)pyrimidin-5-yl)- 2-isopropylpyrimidine- 5-carboxamide |
| 29 | 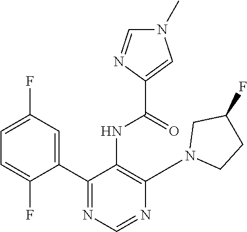 [SMILES: Cn1cnc(C(=O)Nc2c(-c3cc(F)ccc3F)ncnc2N2CC[C@H](F)C2)c1] | (S)-N-(4-(2,5-difluoro- phenyl)-6-(3-fluoropyr- rolidin-1-yl)pyrimidin-5- yl)-1-methyl-1H-imid- azole-4-carboxamide |
| 30 | 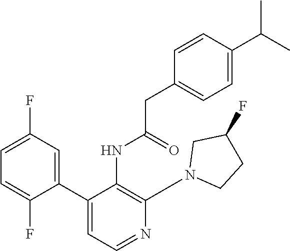 [SMILES: CC(C)c1ccc(CC(=O)Nc2c(-c3cc(F)ccc3F)ccnc2N2CC[C@H](F)C2)cc1] | (S)-N-(4-(2,5-difluoro- phenyl)-2-(3-fluoropyr- rolidin-1-yl)pyridin-3- yl)-2-(4-isopropylphen- yl)acetamide |
| 31 | 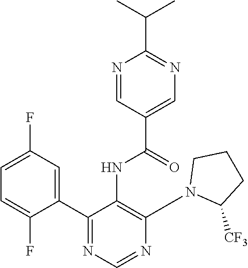 [SMILES: CC(C)c1ncc(C(=O)Nc2c(-c3cc(F)ccc3F)ncnc2N2CCC[C@@H]2C(F)(F)F)cn1] | (R)-N-(4-(2,5-difluoro- phenyl)-6-(2-(trifluoro- methyl)pyrrolidin-1-yl)- pyrimidin-5-yl)-2-iso- propylpyrimidine-5- carboxamide |
| 32 | 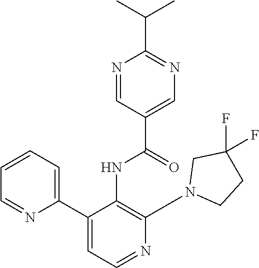 [SMILES: CC(C)c1ncc(C(=O)Nc2c(-c3ccccn3)ccnc2N2CCC(F)(F)C2)cn1] | N-(2′-(3,3-difluoropyr- rolidin-1-yl)-[2,4′-bipyr- idin]-3′-yl)-2-isopropyl- pyrimidine-5-carbox- amide |
| 33 | 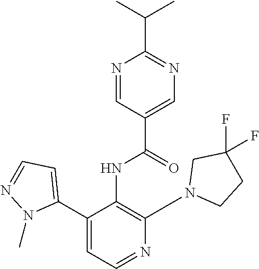 [SMILES: CC(C)c1ncc(C(=O)Nc2c(-c3ccnn3C)ccnc2N2CCC(F)(F)C2)cn1] | N-(2-(3,3-difluoropyr- rolidin-1-yl)-4-(1-meth- yl-1H-pyrazol-5-yl)pyr- idin-3-yl)-2-isopropyl- pyrimidine-5-carbox- amide |
| 34 | 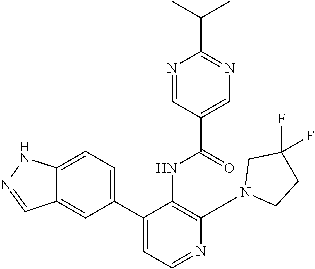 [SMILES: CC(C)c1ncc(C(=O)Nc2c(-c3ccc4[nH]ncc4c3)ccnc2N2CCC(F)(F)C2)cn1] | N-(2-(3,3-difluoropyr- rolidin-1-yl)-4-(1H- indazol-5-yl)pyridin-3- yl)-2-isopropylpyrim- idine-5-carboxamide |
| 35 | 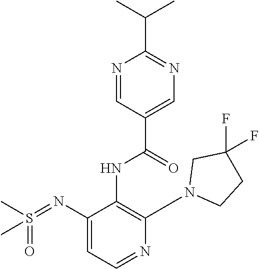 [SMILES: CC(C)c1ncc(C(=O)Nc2c(N=S(C)(C)=O)ccnc2N2CCC(F)(F)C2)cn1] | N-(2-(3,3-difluoropyr- rolidin-1-yl)-4-((dimeth- yl(oxo)-lambda6-sulfane- ylidene)amino)pyridin- 3-yl)-2-isopropylpyrimi- dine-5-carboxamide |
| 36 | 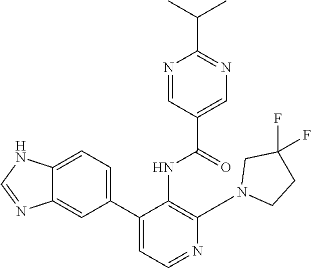 [SMILES: CC(C)c1ncc(C(=O)Nc2c(-c3ccc4[nH]cnc4c3)ccnc2N2CCC(F)(F)C2)cn1] | N-(4-(1H-benzo[d]imid- azol-5-yl)-2-(3,3-di- fluoropyrrolidin-1-yl)- pyridin-3-yl)-2-isoprop- ylpyrimidine-5-carbox- amide |
| 37 | 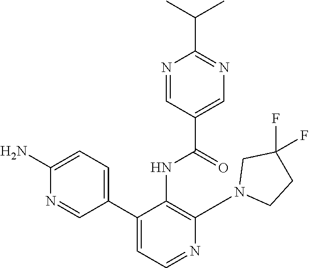 [SMILES: CC(C)c1ncc(C(=O)Nc2c(-c3ccc(N)nc3)ccnc2N2CCC(F)(F)C2)cn1] | N-(6-amino-2′-(3,3-di- fluoropyrrolidin-1-yl)- [3,4′-bipyridin]-3′-yl)-2- isopropylpyrimidine-5- carboxamide |
| 38 | 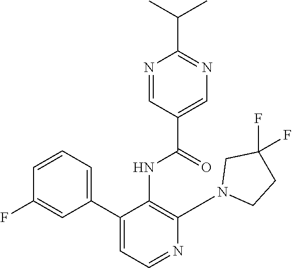 [SMILES: CC(C)c1ncc(C(=O)Nc2c(-c3cccc(F)c3)ccnc2N2CCC(F)(F)C2)cn1] | N-(2-(3,3-difluoropyr- rolidin-1-yl)-4-(3-fluoro- phenyl)pyridin-3-yl)-2- isopropylpyrimidine-5- carboxamide |
| 39 | 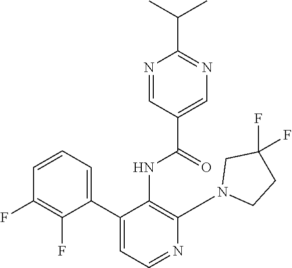 [SMILES: CC(C)c1ncc(C(=O)Nc2c(-c3cccc(F)c3F)ccnc2N2CCC(F)(F)C2)cn1] | N-(4-(2,3-difluorophen- yl)-2-(3,3-difluoropyr- rolidin-1-yl)pyridin-3- yl)-2-isopropylpyrim- idine-5-carboxamide |
| 40 | 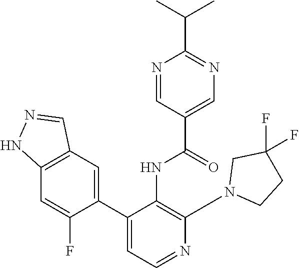 [SMILES: CC(C)c1ncc(C(=O)Nc2c(-c3cc4cn[nH]c4cc3F)ccnc2N2CCC(F)(F)C2)cn1] | N-(2-(3,3-difluoropyr- rolidin-1-yl)-4-(6-fluoro- 1H-indazol-5-yl)pyridin- 3-yl)-2-isopropylpyrim- idine-5-carboxamide |
| 41 | 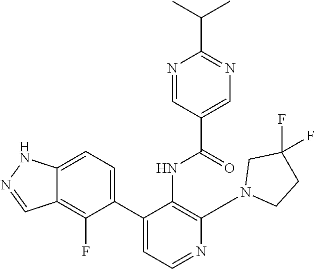 [SMILES: CC(C)c1ncc(C(=O)Nc2c(-c3ccc4[nH]ncc4c3F)ccnc2N2CCC(F)(F)C2)cn1] | N-(2-(3,3-difluoropyr- rolidin-1-yl)-4-(4-fluoro- 1H-indazol-5-yl)pyridin- 3-yl)-2-isopropylpyrim- idine-5-carboxamide |
| 42 | 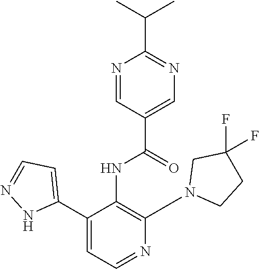 [SMILES: CC(C)c1ncc(C(=O)Nc2c(-c3ccn[nH]3)ccnc2N2CCC(F)(F)C2)cn1] | N-(2-(3,3-difluoropyr- rolidin-1-yl)-4-(1H-pyr- azol-5-yl)pyridin-3-yl)- 2-isopropylpyrimidine- 5-carboxamide |
| 43 | 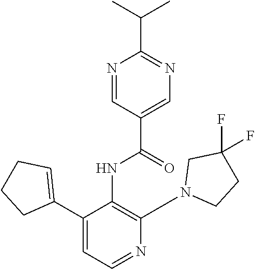 [SMILES: CC(C)c1ncc(C(=O)Nc2c(C3=CCCC3)ccnc2N2CCC(F)(F)C2)cn1] | N-(4-(cyclopent-1-en-1- yl)-2-(3,3-difluoropyr- rolidin-1-yl)pyridin-3- yl)-2-isopropylpyrimi- dine-5-carboxamide |
| 44 | 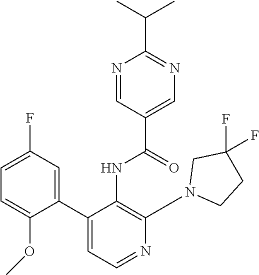 [SMILES: COc1ccc(F)cc1-c1ccnc(N2CCC(F)(F)C2)c1NC(=O)c1cnc(C(C)C)nc1] | N-(2-(3,3-difluoropyr- rolidin-1-yl)-4-(5-fluoro- 2-methoxyphenyl)pyri- din-3-yl)-2-isopropylpyr- imidine-5-carboxamide |
| 45 | 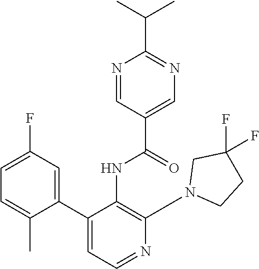 [SMILES: Cc1ccc(F)cc1-c1ccnc(N2CCC(F)(F)C2)c1NC(=O)c1cnc(C(C)C)nc1] | N-(2-(3,3-difluoropyr- rolidin-1-yl)-4-(5-fluoro- 2-methylphenyl)pyridin- 3-yl)-2-isopropylpyrimi- dine-5-carboxamide |
| 46 | 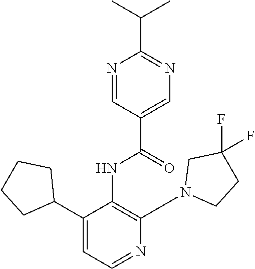 [SMILES: CC(C)c1ncc(C(=O)Nc2c(C3CCCC3)ccnc2N2CCC(F)(F)C2)cn1] | N-(4-cyclopentyl-2-(3,3- difluoropyrrolidin-1-yl)- pyridin-3-yl)-2-isoprop- ylpyrimidine-5-carbox- amide |
| 47 | 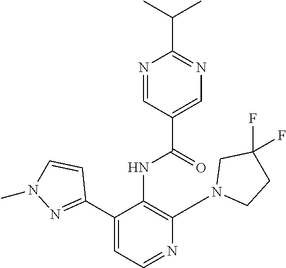 [SMILES: CC(C)c1ncc(C(=O)Nc2c(-c3ccn(C)n3)ccnc2N2CCC(F)(F)C2)cn1] | N-(2-(3,3-difluoropyr- rolidin-1-yl)-4-(1-meth- yl-1H-pyrazol-3-yl)pyr- idin-3-yl)-2-isopropyl- pyrimidine-5-carbox- amide |
| 48 | 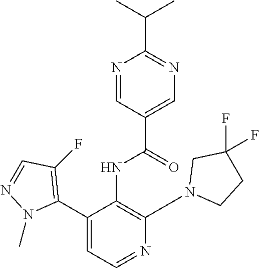 [SMILES: CC(C)c1ncc(C(=O)Nc2c(-c3c(F)cnn3C)ccnc2N2CCC(F)(F)C2)cn1] | N-(2-(3,3-difluoropyr- rolidin-1-yl)-4-(4-fluoro- 1-methyl-1H-pyrazol-5- yl)pyridin-3-yl)-2-iso- propylpyrimidine-5- carboxamide |
| 49 | 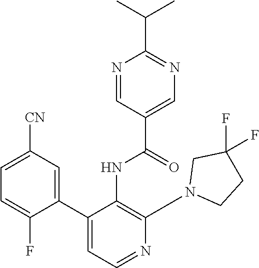 [SMILES: CC(C)c1ncc(C(=O)Nc2c(-c3cc(C#N)ccc3F)ccnc2N2CCC(F)(F)C2)cn1] | N-(4-(5-cyano-2-fluoro- phenyl)-2-(3,3-difluoro- pyrrolidin-1-yl)pyridin- 3-yl)-2-isopropylpyrim- idine-5-carboxamide |
| 50 | 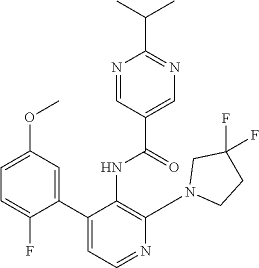 [SMILES: COc1ccc(F)c(-c2ccnc(N3CCC(F)(F)C3)c2NC(=O)c2cnc(C(C)C)nc2)c1] | N-(2-(3,3-difluoropyr- rolidin-1-yl)-4-(2-fluoro- 5-methoxyphenyl)pyri- din-3-yl)-2-isopropylpyr- imidine-5-carboxamide |
| 51 | 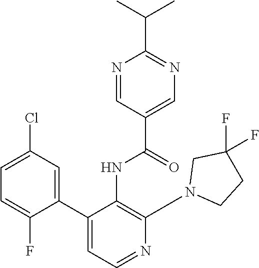 [SMILES: CC(C)c1ncc(C(=O)Nc2c(-c3cc(Cl)ccc3F)ccnc2N2CCC(F)(F)C2)cn1] | N-(4-(5-chloro-2-fluoro- phenyl)-2-(3,3-difluoro- pyrrolidin-1-yl)pyridin- 3-yl)-2-isopropylpyrim- idine-5-carboxamide |
| 52 | 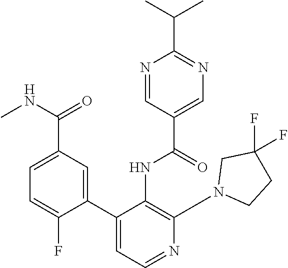 [SMILES: CNC(=O)c1ccc(F)c(-c2ccnc(N3CCC(F)(F)C3)c2NC(=O)c2cnc(C(C)C)nc2)c1] | N-(2-(3,3-difluoropyr- rolidin-1-yl)-4-(2-fluoro- 5-(methylcarbamoyl)- phenyl)pyridin-3-yl)-2- isopropylpyrimidine-5- carboxamide |
| 53 | 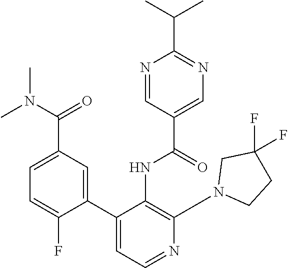 [SMILES: CC(C)c1ncc(C(=O)Nc2c(-c3cc(C(=O)N(C)C)ccc3F)ccnc2N2CCC(F)(F)C2)cn1] | N-(2-(3,3-difluoropyr- rolidin-1-yl)-4-(5- (dimethylcarbamoyl)-2- fluorophenyl)pyridin-3- yl)-2-isopropylpyrim- idine-5-carboxamide |
| 54 | 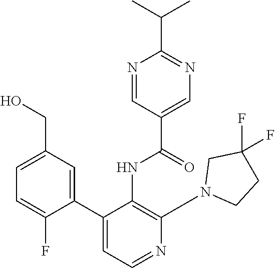 [SMILES: CC(C)c1ncc(C(=O)Nc2c(-c3cc(CO)ccc3F)ccnc2N2CCC(F)(F)C2)cn1] | N-(2-(3,3-difluoropyr- rolidin-1-yl)-4-(2-fluoro- 5-(hydroxymethyl)phen- yl)pyridin-3-yl)-2-iso- propylpyrimidine-5- carboxamide |
| 55 | 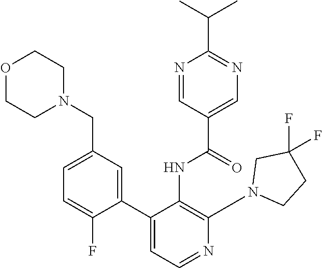 [SMILES: CC(C)c1ncc(C(=O)Nc2c(-c3cc(CN4CCOCC4)ccc3F)ccnc2N2CCC(F)(F)C2)cn1] | N-(2-(3,3-difluoropyr- rolidin-1-yl)-4-(2-fluoro- 5-(morpholinomethyl)- phenyl)pyridin-3-yl)-2- isopropylpyrimidine-5- carboxamide |
| 56 | 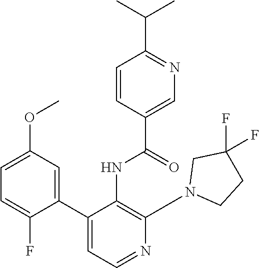 [SMILES: COc1ccc(F)c(-c2ccnc(N3CCC(F)(F)C3)c2NC(=O)c2ccc(C(C)C)nc2)c1] | N-[2-(3,3-difluoropyr- rolidin-1-yl)-4-(2-fluoro- 5-methoxy-phenyl)-3- pyridyl]-6-isopropyl- pyridine-3-carboxamide |
| 57 | 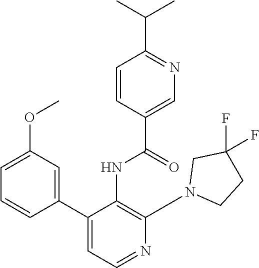 [SMILES: COc1cccc(-c2ccnc(N3CCC(F)(F)C3)c2NC(=O)c2ccc(C(C)C)nc2)c1] | N-[2-(3,3-difluoropyr- rolidin-1-yl)-4-(3-meth- oxyphenyl)-3-pyridyl]- 2-isopropyl-pyrimidine- 5-carboxamide |
| 58 | 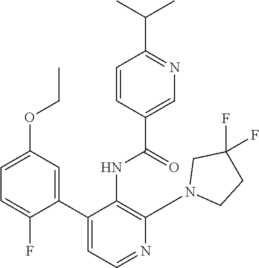 [SMILES: CCOc1ccc(F)c(-c2ccnc(N3CCC(F)(F)C3)c2NC(=O)c2ccc(C(C)C)nc2)c1] | N-[2-(3,3-difluoropyr- rolidin-1-yl)-4-(5-ethoxy- 2-fluorophenyl)-3-pyr- idyl]-2-isopropyl-pyrim- idine-5-carboxamide |
| 59 | 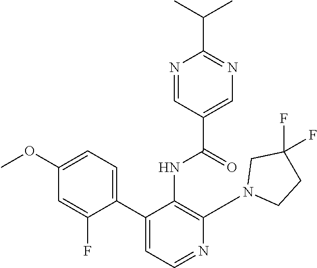 [SMILES: COc1ccc(-c2ccnc(N3CCC(F)(F)C3)c2NC(=O)c2cnc(C(C)C)nc2)c(F)c1] | N-[2-(3,3-difluoropyr- rolidin-1-yl)-4-(2-fluoro- 4-methoxy-phenyl)-3- pyridyl]-2-isopropyl-pyr- imidine-5-carboxamide |
| 60 | 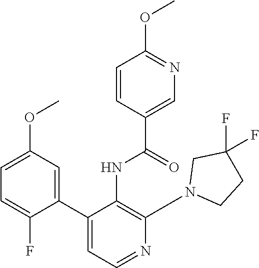 [SMILES: COc1ccc(F)c(-c2ccnc(N3CCC(F)(F)C3)c2NC(=O)c2ccc(OC)nc2)c1] | N-[2-(3,3-difluoropyr- rolidin-1-yl)-4-(2-fluoro- 5-methoxy-phenyl)-3- pyridyl]-6-methoxy-pyr- idine-3-carboxamide |
| 61 | 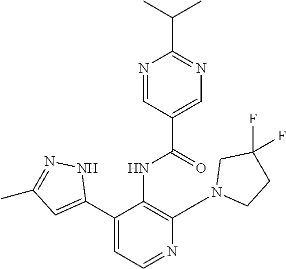 [SMILES: Cc1cc(-c2ccnc(N3CCC(F)(F)C3)c2NC(=O)c2cnc(C(C)C)nc2)[nH]n1] | N-(2-(3,3-difluoropyr- rolidin-1-yl)-4-(3-meth- yl-1H-pyrazol-5-yl)pyr- idin-3-yl)-2-isopropyl- pyrimidine-5-carbox- amide |
| 62 | 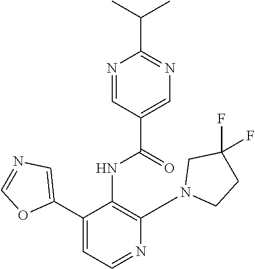 [SMILES: CC(C)c1ncc(C(=O)Nc2c(-c3cnco3)ccnc2N2CCC(F)(F)C2)cn1] | N-(2-(3,3-difluoropyr- rolidin-1-yl)-4-(oxazol- 5-yl)pyridin-3-yl)-2-iso- propylpyrimidine-5- carboxamide |
| 63 | 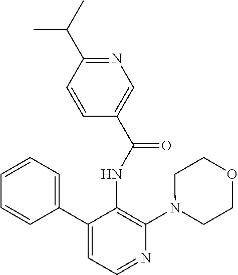 [SMILES: CC(C)c1ccc(C(=O)Nc2c(-c3ccccc3)ccnc2N2CCOCC2)cn1] | 6-isopropyl-N-(2-mor- pholino-4-phenyl-pyri- din-3-yl)nicotinamide |
| 64 | 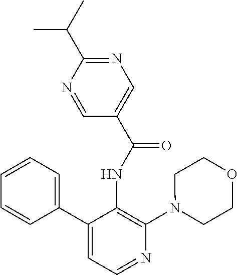 [SMILES: CC(C)c1ncc(C(=O)Nc2c(-c3ccccc3)ccnc2N2CCOCC2)cn1] | 2-isopropyl-N-(2-morph- olino-4-phenylpyridin-3- yl)pyrimidine-5-carbox- amide |
| 65 | 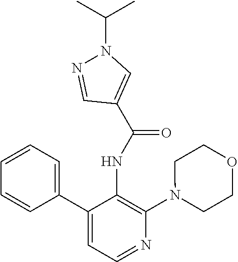 [SMILES: CC(C)n1cc(C(=O)Nc2c(-c3ccccc3)ccnc2N2CCOCC2)cn1] | 1-isopropyl-N-(2-mor- pholino-4-phenylpyridin- 3-yl)-1H-pyrazole-4- carboxamide |
| 66 | 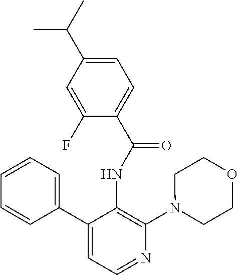 [SMILES: CC(C)c1ccc(C(=O)Nc2c(-c3ccccc3)ccnc2N2CCOCC2)c(F)c1] | 2-fluoro-4-isopropyl-N- (2-morpholino-4-phen- ylpyridin-3-yl)benzamide |
| 67 | 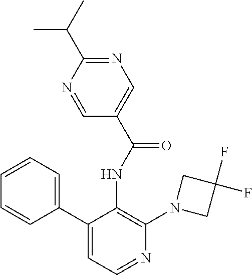 [SMILES: CC(C)c1ncc(C(=O)Nc2c(-c3ccccc3)ccnc2N2CC(F)(F)C2)cn1] | N-(2-(3,3-difluoroazeti- din-1-yl)-4-phenylpyri- din-3-yl)-2-isopropylpyr- imidine-5-carboxamide |
| 68 | 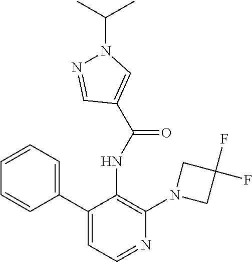 [SMILES: CC(C)n1cc(C(=O)Nc2c(-c3ccccc3)ccnc2N2CC(F)(F)C2)cn1] | N-(2-(3,3-difluoroazeti- din-1-yl)-4-phenylpyri- din-3-yl)-1-isopropyl- 1H-pyrazole-4-carbox- amide |
| 69 | 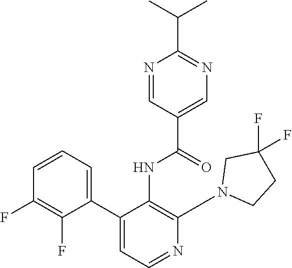 [SMILES: CC(C)c1ncc(C(=O)Nc2c(-c3cccc(F)c3F)ccnc2N2CCC(F)(F)C2)cn1] | 2-isopropyl-N-(4-phenyl- 2-(2-oxa-6-azaspiro[3.3] heptan-6-yl)pyridin-3- yl)pyrimidine-5-carbox- amide |
| 70 | 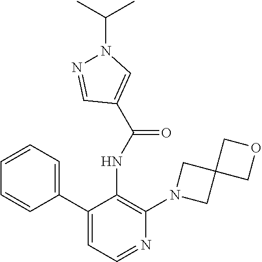 [SMILES: CC(C)n1cc(C(=O)Nc2c(-c3ccccc3)ccnc2N2CC3(COC3)C2)cn1] | 1-isopropyl-N-(4-phenyl- 2-(2-oxa-6-azaspiro[3.3] heptan-6-yl)pyridin-3- yl)-1H-pyrazole-4-car- boxamide |
| 71 | 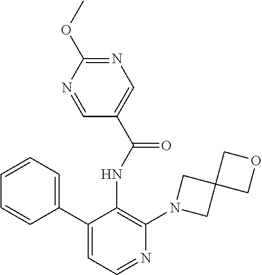 [SMILES: COc1ncc(C(=O)Nc2c(-c3ccccc3)ccnc2N2CC3(COC3)C2)cn1] | 2-methoxy-N-(4-phenyl- 2-(2-oxa-6-azaspiro[3.3] heptan-6-yl)pyridin-3- yl)pyrimidine-5-carbox- amide |
| 72 | 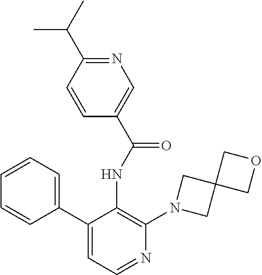 [SMILES: CC(C)c1ccc(C(=O)Nc2c(-c3ccccc3)ccnc2N2CC3(COC3)C2)cn1] | 6-isopropyl-N-(4-phen- yl-2-(2-oxa-6-azaspiro [3.3]heptan-6-yl)pyridin- 3-yl)nicotinamide |
| 73 | 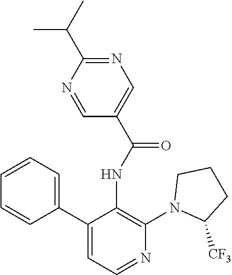 [SMILES: CC(C)c1ncc(C(=O)Nc2c(-c3ccccc3)ccnc2N2CCC[C@@H]2C(F)(F)F)cn1] | (R)-2-isopropyl-N-(4- phenyl-2-(2-(trifluoro- methyl)pyrrolidin-1-yl)- pyridin-3-yl)pyrimidine- 5-carboxamide |
| 74 | 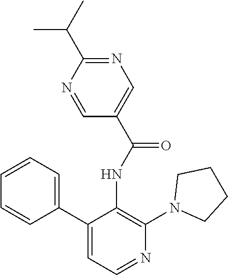 [SMILES: CC(C)c1ncc(C(=O)Nc2c(-c3ccccc3)ccnc2N2CCCC2)cn1] | 2-isopropyl-N-(4-phen- yl-2-(pyrrolidin-1-yl)- pyridin-3-yl)pyrimidine- 5-carboxamide |
| 75 | 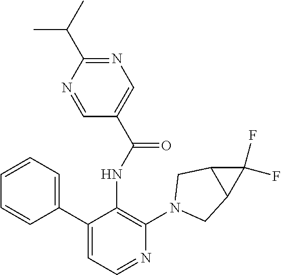 [SMILES: CC(C)c1ncc(C(=O)Nc2c(-c3ccccc3)ccnc2N2CC3C(C2)C3(F)F)cn1] | N-(2-(6,6-difluoro-3- azabicyclo[3.1.0]hexan- 3-yl)-4-phenylpyridin-3- yl)-2-isopropylpyrimi- dine-5-carboxamide |
| 76 | 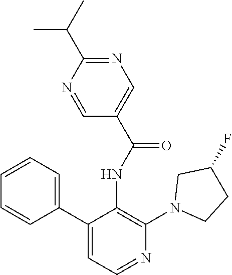 [SMILES: CC(C)c1ncc(C(=O)Nc2c(-c3ccccc3)ccnc2N2CC[C@@H](F)C2)cn1] | (S)-N-(2-(3-fluoropyroli- din-1-yl)-4-phenylpyri- din-3-yl)-2-isopropylpyr- imidine-5-carboxamide |
| 77 | 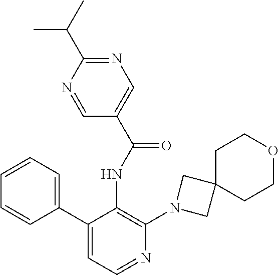 [SMILES: CC(C)c1ncc(C(=O)Nc2c(-c3ccccc3)ccnc2N2CC3(CCOCC3)C2)cn1] | 2-isopropyl-N-(4-phen- yl-2-(7-oxa-2-azaspiro [3.5]nonan-2-yl)pyridin- 3-yl)pyrimidine-5- carboxamide |
| 78 | 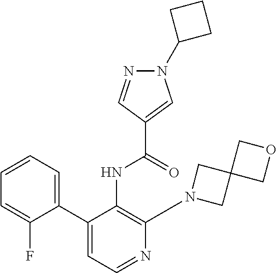 [SMILES: O=C(Nc1c(-c2ccccc2F)ccnc1N1CC2(COC2)C1)c1cnn(C2CCC2)c1] | 1-cyclobutyl-N-(4-(2- fluorophenyl)-2-(2-oxa- 6-azaspiro[3.3]heptan-6- yl)pyridin-3-yl)-1H-pyr- azole-4-carboxamide |
| 79 | 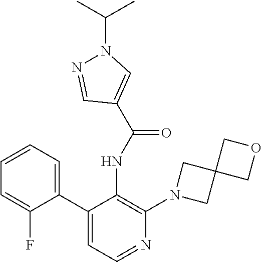 [SMILES: CC(C)n1cc(C(=O)Nc2c(-c3ccccc3F)ccnc2N2CC3(COC3)C2)cn1] | N-(4-(2-fluorophenyl)-2- (2-oxa-6-azaspiro[3.3] heptan-6-yl)pyridin-3- yl)-1-isopropyl-1H- pyrazole-4-carboxamide |
| 80 | 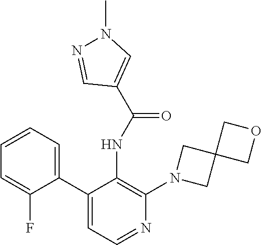 [SMILES: Cn1cc(C(=O)Nc2c(-c3ccccc3F)ccnc2N2CC3(COC3)C2)cn1] | N-(4-(2-fluorophenyl)-2- (2-oxa-6-azaspiro[3.3] heptan-6-yl)pyridin-3- yl)-1-methyl-1H-pyr- azole-4-carboxamide |
| 81 | 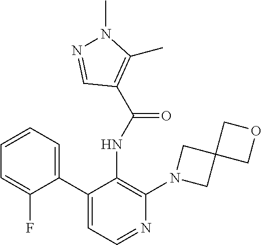 [SMILES: Cc1c(C(=O)Nc2c(-c3ccccc3F)ccnc2N2CC3(COC3)C2)cnn1C] | N-(4-(2-fluorophenyl)-2- (2-oxa-6-azaspiro[3.3] heptan-6-yl)pyridin-3- yl)-1,5-dimethyl-1H- pyrazole-4-carboxamide |
| 82 | 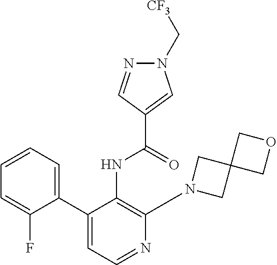 [SMILES: O=C(Nc1c(-c2ccccc2F)ccnc1N1CC2(COC2)C1)c1cnn(CC(F)(F)F)c1] | N-(4-(2-fluorophenyl)-2- (2-oxa-6-azaspiro[3.3] heptan-6-yl)pyridin-3-yl)- 1-(2,2,2-trifluoroethyl)- 1H-pyrazole-4-carbox- amide |
| 83 | 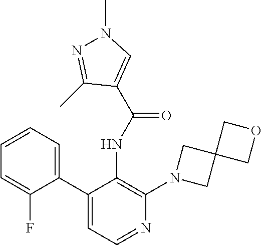 [SMILES: Cc1nn(C)cc1C(=O)Nc1c(-c2ccccc2F)ccnc1N1CC2(COC2)C1] | N-(4-(2-fluorophenyl)-2- (2-oxa-6-azaspiro[3.3] heptan-6-yl)pyridin-3- yl)-1,3-dimethyl-1H- pyrazole-4-carboxamide |
| 84 | 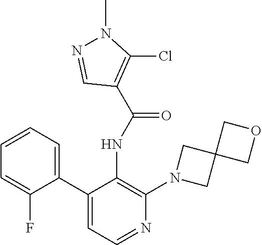 [SMILES: Cn1ncc(C(=O)Nc2c(-c3ccccc3F)ccnc2N2CC3(COC3)C2)c1Cl] | 5-chloro-N-(4-(2-fluoro- phenyl)-2-(2-oxa-6-aza- spiro[3.3]heptan-6-yl)- pyridin-3-yl)-1-methyl- 1H-pyrazole-4-carbox- amide |
| 85 | 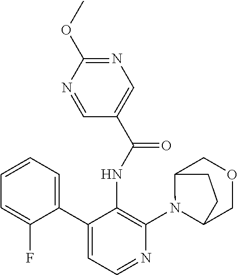 [SMILES: COc1ncc(C(=O)Nc2c(-c3ccccc3F)ccnc2N2C3CCC2COC3)cn1] | N-(2-(3-oxa-8-azabicyclo [3.2.1]octan-8-yl)-4-(2- fluorophenyl)pyridin-3- yl)-2-methoxypyrimi- dine-5-carboxamide |
| 86 | 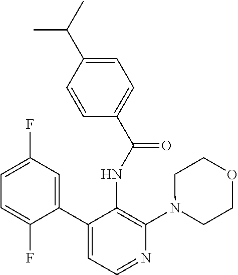 [SMILES: CC(C)c1ccc(C(=O)Nc2c(-c3cc(F)ccc3F)ccnc2N2CCOCC2)cc1] | N-(4-(2,5-difluorophen- yl)-2-morpholinopyridin- 3-yl)-4-isopropylbenz- amide |
| 87 | 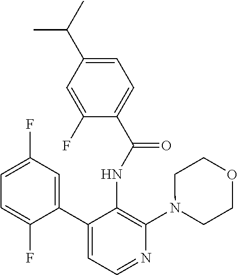 [SMILES: CC(C)c1ccc(C(=O)Nc2c(-c3cc(F)ccc3F)ccnc2N2CCOCC2)c(F)c1] | N-(4-(2,5-difluorophen- yl)-2-morpholinopyridin- 3-yl)-2-fluoro-4-isoprop- ylbenzamide |
| 88 | 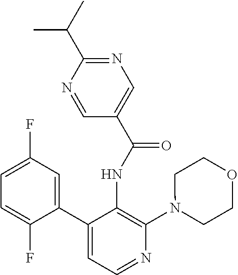 [SMILES: CC(C)c1ncc(C(=O)Nc2c(-c3cc(F)ccc3F)ccnc2N2CCOCC2)cn1] | N-(4-(2,5-difluorophen- yl)-2-morpholinopyridin- 3-yl)-2-isopropylpyrimi- dine-5-carboxamide |
| 89 | 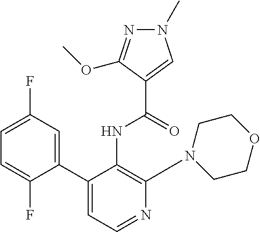 [SMILES: COc1nn(C)cc1C(=O)Nc1c(-c2cc(F)ccc2F)ccnc1N1CCOCC1] | N-(4-(2,5-difluorophen- yl)-2-morpholinopyridin- 3-yl)-3-methoxy-1-meth- yl-1H-pyrazole-4-carbox- amide |
| 90 | 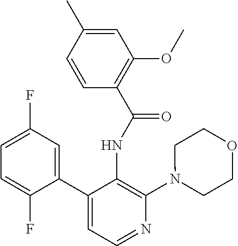 [SMILES: COc1cc(C)ccc1C(=O)Nc1c(-c2cc(F)ccc2F)ccnc1N1CCOCC1] | N-(4-(2,5-difluorophen- yl)-2-morpholinopyridin- 3-yl)-2-methoxy-4-meth- ylbenzamide |
| 91 | 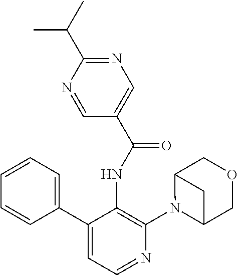 [SMILES: CC(C)c1ncc(C(=O)Nc2c(-c3ccccc3)ccnc2N2C3COCC2C3)cn1] | N-(2-(3-oxa-6-azabicyclo [3.1.1]heptan-6-yl)-4- phenylpyridin-3-yl)-2- isopropylpyrimidine-5- carboxamide |
| 92 | 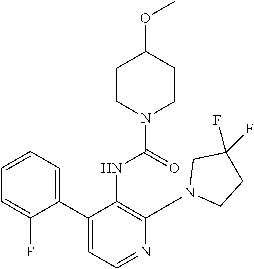 [SMILES: COC1CCN(C(=O)Nc2c(-c3ccccc3F)ccnc2N2CCC(F)(F)C2)CC1] | N-(2-(3,3-difluoropyr- rolidin-1-yl)-4-(2-fluoro- phenyl)pyridin-3-yl)-4- methoxypiperidine-1- carboxamide |
| 93 | 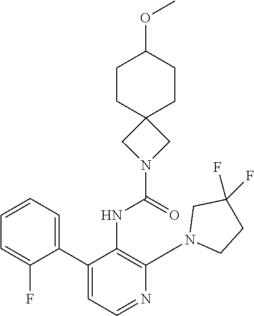 [SMILES: COC1CCC2(CC1)CN(C(=O)Nc1c(-c3ccccc3F)ccnc1N1CCC(F)(F)C1)C2] | N-(2-(3,3-difluoropyr- rolidin-1-yl)-4-(2-fluoro- phenyl)pyridin-3-yl)-7- methoxy-2-azaspiro[3.5] nonane-2-carboxamide |
| 94 | 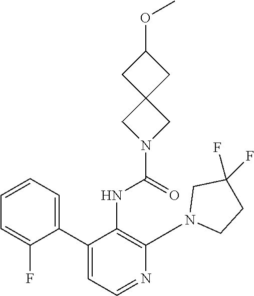 [SMILES: COC1CC2(C1)CN(C(=O)Nc1c(-c3ccccc3F)ccnc1N1CCC(F)(F)C1)C2] | N-(2-(3,3-difluoropyr- rolidin-1-yl)-4-(2-fluoro- phenyl)pyridin-3-yl)-6- methoxy-2-azaspiro[3.3] heptane-2-carboxamide |
| 95 | 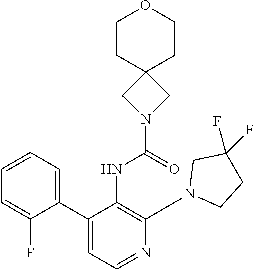 [SMILES: O=C(Nc1c(-c2ccccc2F)ccnc1N1CCC(F)(F)C1)N1CC2(CCOCC2)C1] | N-(2-(3,3-difluoropyr- rolidin-1-yl)-4-(2-fluoro- phenyl)pyridin-3-yl)-7- oxa-2-azaspiro[3.5] nonane-2-carboxamide |
| 96 | 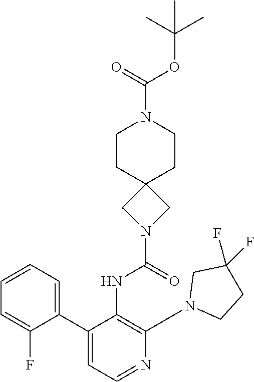 [SMILES: CC(C)(C)OC(=O)N1CCC2(CC1)CN(C(=O)Nc1c(-c3ccccc3F)ccnc1N1CCC(F)(F)C1)C2] | tert-butyl 2-((2-(3,3- difluoropyrrolidin-1-yl)- 4-(2-fluorophenyl)pyri- din-3-yl)carbamoyl)-2,7- diazaspiro[3.5]nonane- 7-carboxylate |
| 97 | 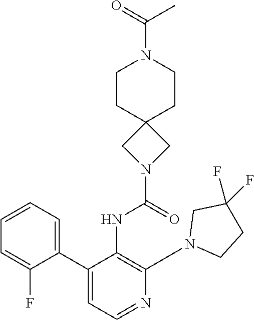 [SMILES: CC(=O)N1CCC2(CC1)CN(C(=O)Nc1c(-c3ccccc3F)ccnc1N1CCC(F)(F)C1)C2] | 7-acetyl-N-(2-(3,3- difluoropyrrolidin-1-yl)- 4-(2-fluorophenyl)pyri- din-3-yl)-2,7-diazaspiro [3.5]nonane-2-carbox- amide |
| 98 | 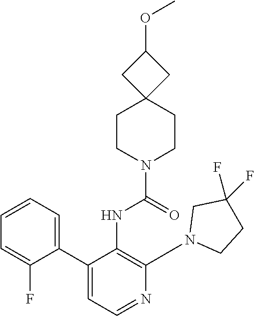 [SMILES: COC1CC2(CCN(C(=O)Nc3c(-c4ccccc4F)ccnc3N3CCC(F)(F)C3)CC2)C1] | N-(2-(3,3-difluoropyr- rolidin-1-yl)-4-(2-fluoro- phenyl)pyridin-3-yl)-2- methoxy-7-azaspiro[3.5] nonane-7-carboxamide |
| 99 | 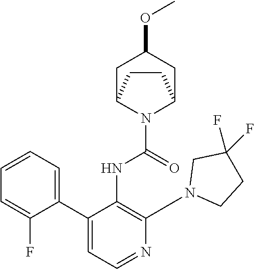 [SMILES: CO[C@@H]1C[C@H]2CC[C@@H](C1)N2C(=O)Nc1c(-c2ccccc2F)ccnc1N1CCC(F)(F)C1] | (1R,3s,5S)-N-(2-(3,3- difluoropyrrolidin-1-yl)- 4-(2-fluorophenyl)pyri- din-3-yl)-3-methoxy-8- azabicyclo[3.2.1]octane- 8-carboxamide |
| 100 | 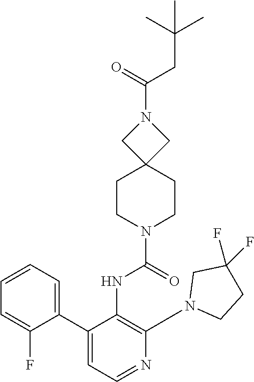 [SMILES: CC(C)(C)CC(=O)N1CC2(CCN(C(=O)Nc3c(-c4ccccc4F)ccnc3N3CCC(F)(F)C3)CC2)C1] | N-[2-(3,3-difluoropyr- rolidin-1-yl)-4-(2-fluoro- phenyl)-3-pyridyl]-2- (3,3-dimethylbutanoyl)- 2,7-diazaspiro[3.5] nonane-7-carboxamide |
| 101 | 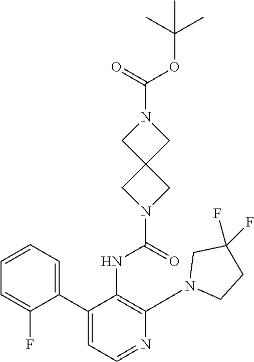 [SMILES: CC(C)(C)OC(=O)N1CC2(CN(C(=O)Nc3c(-c4ccccc4F)ccnc3N3CCC(F)(F)C3)C2)C1] | tert-butyl 6-((2-(3,3- difluoropyrrolidin-1-yl)- 4-(2-fluorophenyl)pyri- din-3-yl)carbamoyl)-2,6- diazaspiro[3.3]heptane- 2-carboxylate |
| 102 | 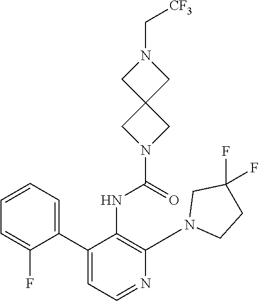 [SMILES: O=C(Nc1c(-c2ccccc2F)ccnc1N1CCC(F)(F)C1)N1CC2(CN(CC(F)(F)F)C2)C1] | N-(2-(3,3-difluoropyr- rolidin-1-yl)-4-(2-fluoro- phenyl)pyridin-3-yl)-6- (2,2,2-trifluoroethyl)- 2,6-diazaspiro[3.3] heptane-2-carboxamide |
| 103 | 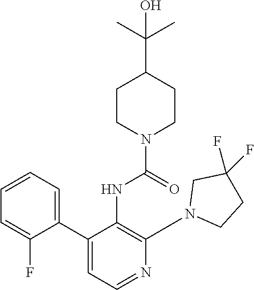 [SMILES: CC(C)(O)C1CCN(C(=O)Nc2c(-c3ccccc3F)ccnc2N2CCC(F)(F)C2)CC1] | N-[2-(3,3-difluoropyr- rolidin-1-yl)-4-(2-fluoro- phenyl)-3-pyridyl]-4-(1- hydroxy-1-methyl-ethyl)- piperidine-1-carboxamide |
| 104 | 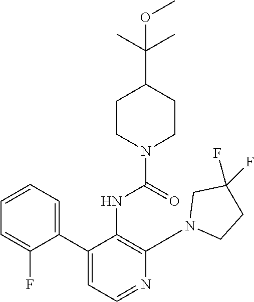 [SMILES: COC(C)(C)C1CCN(C(=O)Nc2c(-c3ccccc3F)ccnc2N2CCC(F)(F)C2)CC1] | N-[2-(3,3-difluoropyr- rolidin-1-yl)-4-(2-fluoro- phenyl)-3-pyridyl]-4-(1- methoxy-1-methyl-ethyl)- piperidine-1-carboxamide |
| 105 | 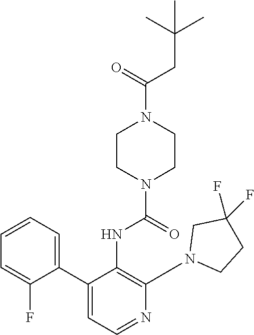 [SMILES: CC(C)(C)CC(=O)N1CCN(C(=O)Nc2c(-c3ccccc3F)ccnc2N2CCC(F)(F)C2)CC1] | N-[2-(3,3-difluoropyr- rolidin-1-yl)-4-(2-fluoro- phenyl)-3-pyridyl]-4- (2,2-dimethylpropyl)-3- oxo-piperazine-1-carbox- amide |
| 106 | 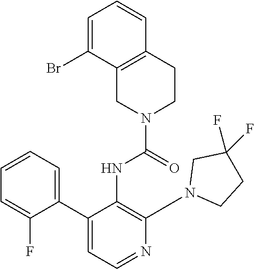 [SMILES: O=C(Nc1c(-c2ccccc2F)ccnc1N1CCC(F)(F)C1)N1CCc2cccc(Br)c2C1] | 8-bromo-N-[2-(3,3- difluoropyrrolidin-1-yl)- 4-(2-fluorophenyl)-3- pyridyl]-3,4-dihydro-1H- isoquinoline-2-carbox- amide |
| 107 | 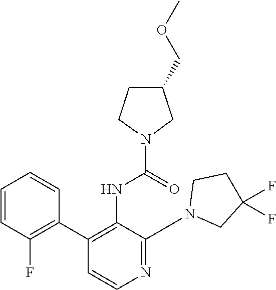 [SMILES: COC[C@H]1CCN(C(=O)Nc2c(-c3ccccc3F)ccnc2N2CCC(F)(F)C2)C1] | (3S)-N-[2-(3,3-difluoro- pyrrolidin-1-yl)-4-(2- fluorophenyl)-3-pyridyl]- 3-(methoxymethyl)pyr- rolidine-1-carboxamide |
| 108 | 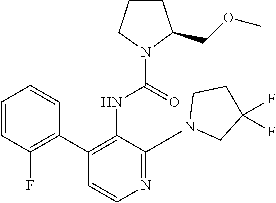 [SMILES: COC[C@@H]1CCCN1C(=O)Nc1c(-c2ccccc2F)ccnc1N1CCC(F)(F)C1] | (2S)-N-[2-(3,3-difluoro- pyrrolidin-1-yl)-4-(2- fluorophenyl)-3-pyridyl]- 2-(methoxymethyl)pyr- rolidine-1-carboxamide |
| 109 | 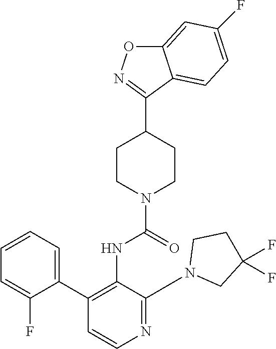 [SMILES: O=C(Nc1c(-c2ccccc2F)ccnc1N1CCC(F)(F)C1)N1CCC(c2noc3cc(F)ccc23)CC1] | N-[2-(3,3-difluoropyr- rolidin-1-yl)-4-(2-fluoro- phenyl)-3-pyridyl]-4-(6- fluoro-1,2-benzoxazol- 3-yl)piperidine-1-carbox- amide |
| 110 | 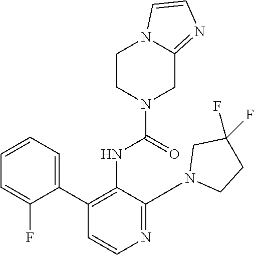 [SMILES: O=C(Nc1c(-c2ccccc2F)ccnc1N1CCC(F)(F)C1)N1CCn2ccnc2C1] | N-[2-(3,3-difluoropyr- rolidin-1-yl)-4-(2-fluoro- phenyl)-3-pyridyl]-6,8- dihydro-5H-imidazo[1,2- a]pyrazine-7-carbox- amide |
| 111 | 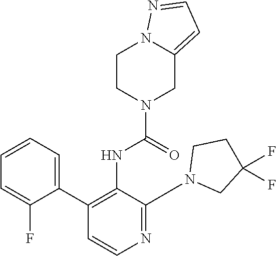 [SMILES: O=C(Nc1c(-c2ccccc2F)ccnc1N1CCC(F)(F)C1)N1CCn2nccc2C1] | N-[2-(3,3-difluoropyr- rolidin-1-yl)-4-(2-fluoro- phenyl)-3-pyridyl]-6,7- dihydro-4H-pyrazolo [1,5-a]pyrazine-5- carboxamide |
| 112 | 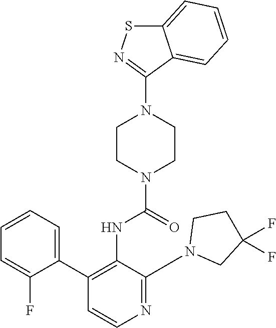 [SMILES: O=C(Nc1c(-c2ccccc2F)ccnc1N1CCC(F)(F)C1)N1CCN(c2nsc3ccccc23)CC1] | 4-(1,2-benzothiazol-3- yl)-N-[2-(3,3-difluoro- pyrrolidin-1-yl)-4-(2- fluorophenyl)-3-pyridyl]- piperazine-1-carbox- amide |
| 113 | 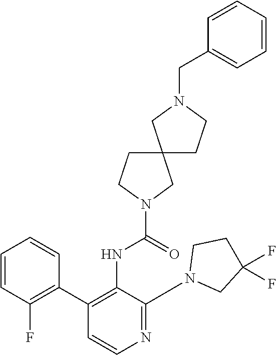 [SMILES: O=C(Nc1c(-c2ccccc2F)ccnc1N1CCC(F)(F)C1)N1CCC2(CCN(Cc3ccccc3)C2)C1] | 7-benzyl-N-[2-(3,3- difluoropyrrolidin-1-yl)- 4-(2-fluorophenyl)-3- pyridyl]-2,7-diazaspiro [4.4]nonane-2-carbox- amide |
| 114 | 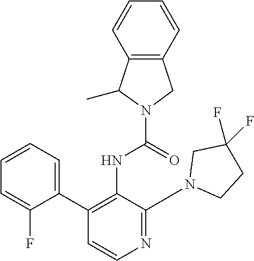 [SMILES: CC1c2ccccc2CN1C(=O)Nc1c(-c2ccccc2F)ccnc1N1CCC(F)(F)C1] | N-[2-(3,3-difluoropyr- rolidin-1-yl)-4-(2-fluoro- phenyl)-3-pyridyl]-1- methyl-isoindoline-2- carboxamide |
| 115 | 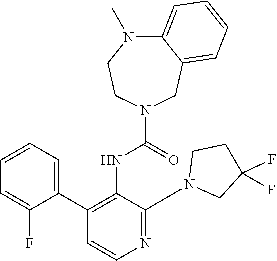 [SMILES: CN1CCN(C(=O)Nc2c(-c3ccccc3F)ccnc2N2CCC(F)(F)C2)Cc2ccccc21] | N-[2-(3,3-difluoropyr- rolidin-1-yl)-4-(2-fluoro- phenyl)-3-pyridyl]-1- methyl-3,5-dihydro-2H- 1,4-benzodiazepine-4- carboxamide |
| 116 | 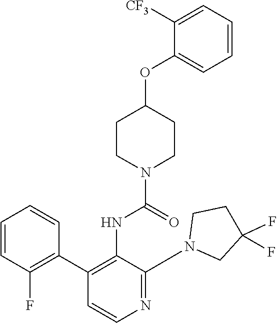 [SMILES: O=C(Nc1c(-c2ccccc2F)ccnc1N1CCC(F)(F)C1)N1CCC(Oc2ccccc2C(F)(F)F)CC1] | N-[2-(3,3-difluoropyrrol- idin-1-yl)-4-(2-fluoro- phenyl)-3-pyridyl]-4-[2- (trifluoromethyl)phen- oxy]piperidine-1-carbox- amide |
| 117 | 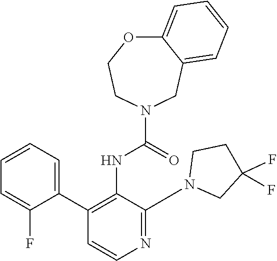 [SMILES: O=C(Nc1c(-c2ccccc2F)ccnc1N1CCC(F)(F)C1)N1CCOc2ccccc2C1] | N-[2-(3,3-difluoropyr- rolidin-1-yl)-4-(2-fluoro- phenyl)-3-pyridyl]-3,5- dihydro-2H-1,4-benzox- azepine-4-carboxamide |
| 118 | 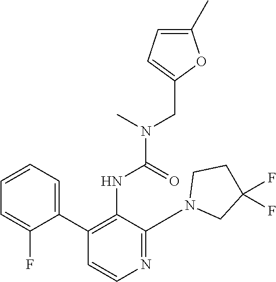 [SMILES: Cc1ccc(CN(C)C(=O)Nc2c(-c3ccccc3F)ccnc2N2CCC(F)(F)C2)o1] | 3-[2-(3,3-difluoropyrrol- idin-1-yl)-4-(2-fluoro- phenyl)-3-pyridyl]-1- methyl-1-[(5-methyl-2- furyl)methyl]urea |
| 119 | 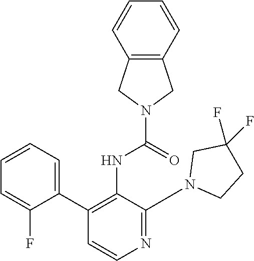 [SMILES: O=C(Nc1c(-c2ccccc2F)ccnc1N1CCC(F)(F)C1)N1Cc2ccccc2C1] | N-[2-(3,3-difluoropyr- rolidin-1-yl)-4-(2-fluoro- phenyl)-3-pyridyl]isoin- doline-2-carboxamide |
| 120 | 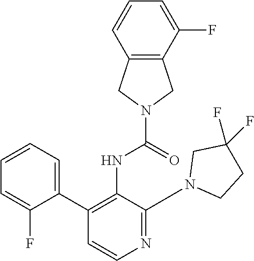 [SMILES: O=C(Nc1c(-c2ccccc2F)ccnc1N1CCC(F)(F)C1)N1Cc2cccc(F)c2C1] | N-[2-(3,3-difluoropyr- rolidin-1-yl)-4-(2-fluoro- phenyl)-3-pyridyl]-4- fluoro-isoindoline-2- carboxamide |
| 121 | 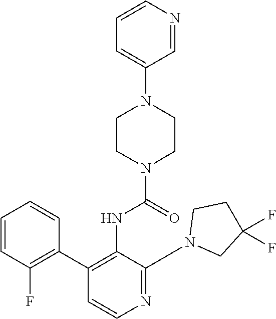 [SMILES: O=C(Nc1c(-c2ccccc2F)ccnc1N1CCC(F)(F)C1)N1CCN(c2cccnc2)CC1] | N-[2-(3,3-difluoropyrrol- idin-1-yl)-4-(2-fluoro- phenyl)-3-pyridyl]-4-(3- pyridyl)piperazine-1- carboxamide |
| 122 | 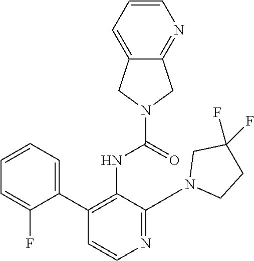 [SMILES: O=C(Nc1c(-c2ccccc2F)ccnc1N1CCC(F)(F)C1)N1Cc2cccnc2C1] | N-[2-(3,3-difluoropyrrol- idin-1-yl)-4-(2-fluoro- phenyl)-3-pyridyl]-5,7- dihydropyrrolo[3,4-b] pyridine-6-carboxamide |
| 123 | 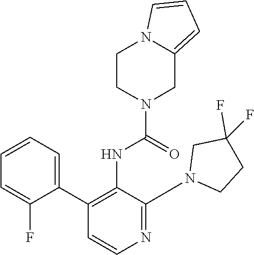 [SMILES: O=C(Nc1c(-c2ccccc2F)ccnc1N1CCC(F)(F)C1)N1CCn2cccc2C1] | N-[2-(3,3-difluoropyr- rolidin-1-yl)-4-(2-fluoro- phenyl)-3-pyridyl]-3,4- dihydro-1H-pyrrolo[1,2- a]pyrazine-2-carbox- amide |
| 124 | 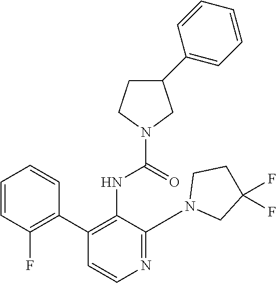 [SMILES: O=C(Nc1c(-c2ccccc2F)ccnc1N1CCC(F)(F)C1)N1CCC(c2ccccc2)C1] | N-[2-(3,3-difluoropyr- rolidin-1-yl)-4-(2-fluoro- phenyl)-3-pyridyl]-3- phenyl-pyrrolidine-1- carboxamide |
| 125 | 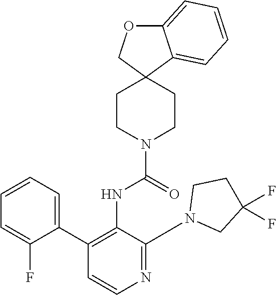 [SMILES: O=C(Nc1c(-c2ccccc2F)ccnc1N1CCC(F)(F)C1)N1CCC2(CC1)COc1ccccc12] | N-[2-(3,3-difluoropyr- rolidin-1-yl)-4-(2-fluoro- phenyl)-3-pyridyl]spiro [2H-benzofuran-3,4′- piperidine]-1′-carbox- amide |
| 126 | 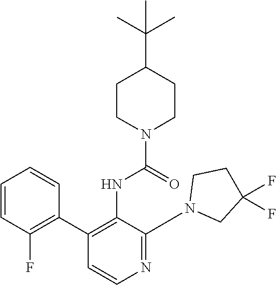 [SMILES: CC(C)(C)C1CCN(C(=O)Nc2c(-c3ccccc3F)ccnc2N2CCC(F)(F)C2)CC1] | 4-tert-butyl-N-[2-(3,3- difluoropyrrolidin-1-yl)- 4-(2-fluorophenyl)-3- pyridyl]piperidine-1- carboxamide |
| 127 | 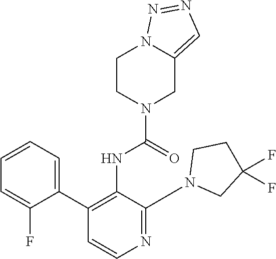 [SMILES: O=C(Nc1c(-c2ccccc2F)ccnc1N1CCC(F)(F)C1)N1CCn2nncc2C1] | N-[2-(3,3-difluoropyr- rolidin-1-yl)-4-(2-fluoro- phenyl)-3-pyridyl]-6,7- dihydro-4H-triazolo[1,5- a]pyrazine-5-carbox- amide |
| 128 | 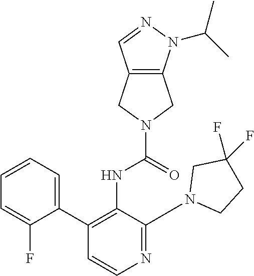 [SMILES: CC(C)n1ncc2c1CN(C(=O)Nc1c(-c3ccccc3F)ccnc1N1CCC(F)(F)C1)C2] | N-[2-(3,3-difluoropyr- rolidin-1-yl)-4-(2-fluoro- phenyl)-3-pyridyl]-1- isopropyl-4,6-dihydro- pyrrolo[3,4-c]pyrazole- 5-carboxamide |
| 129 | 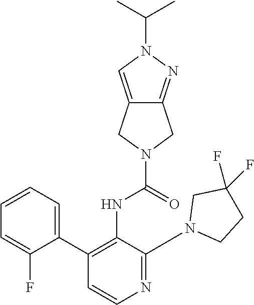 [SMILES: CC(C)n1cc2c(n1)CN(C(=O)Nc1c(-c3ccccc3F)ccnc1N1CCC(F)(F)C1)C2] | N-[2-(3,3-difluoropyrrol- idin-1-yl)-4-(2-fluoro- phenyl)-3-pyridyl]-2- isopropyl-4,6-dihydro- pyrrolo[3,4-c]pyrazole- 5-carboxamide |
| 130 | 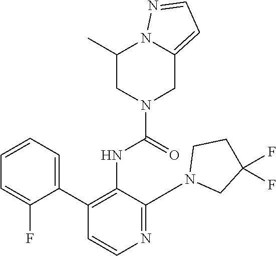 [SMILES: CC1CN(C(=O)Nc2c(-c3ccccc3F)ccnc2N2CCC(F)(F)C2)Cc2ccnn21] | N-[2-(3,3-difluoropyrrol- idin-1-yl)-4-(2-fluoro- phenyl)-3-pyridyl]-7- methyl-6,7-dihydro-4H- pyrazolo[1,5-a]pyrazine- 5-carboxamide |
| 131 | 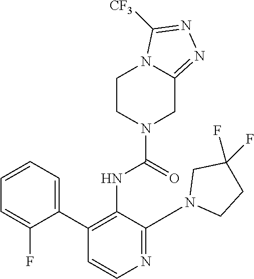 [SMILES: O=C(Nc1c(-c2ccccc2F)ccnc1N1CCC(F)(F)C1)N1CCn2c(nnc2C(F)(F)F)C1] | N-[2-(3,3-difluoropyr- rolidin-1-yl)-4-(2-fluoro- phenyl)-3-pyridyl]-3- (trifluoromethyl)-6,8- dihydro-5H-[1,2,4]tri- azolo[4,3-a]pyrazine-7- carboxamide |
| 132 | 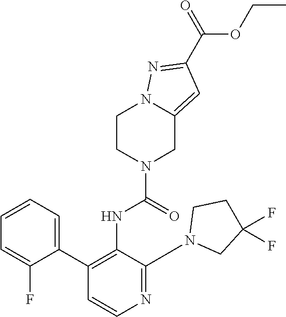 [SMILES: CCOC(=O)c1cc2n(n1)CCN(C(=O)Nc1c(-c3ccccc3F)ccnc1N1CCC(F)(F)C1)C2] | ethyl 5-[2-(3,3-difluoro- pyrrolidin-1-yl)-4-(2- fluorophenyl)-3-pyridyl] carbamoyl]-6,7-dihydro- 4H-pyrazolo[1,5-a]pyr- azine-2-carboxylate |
| 133 | 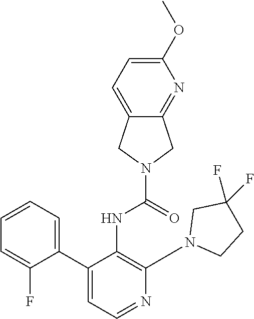 [SMILES: COc1ccc2c(n1)CN(C(=O)Nc1c(-c3ccccc3F)ccnc1N1CCC(F)(F)C1)C2] | N-[2-(3,3-difluoropyr- rolidin-1-yl)-4-(2-fluoro- phenyl)-3-pyridyl]-2- methoxy-5,7-dihydropyr- rolo[3,4-b]pyridine-6- carboxamide |
| 134 | 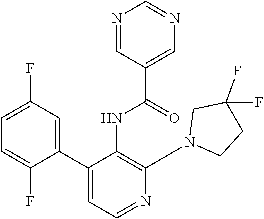 [SMILES: O=C(Nc1c(-c2cc(F)ccc2F)ccnc1N1CCC(F)(F)C1)c1cncnc1] | N-(4-(2,5-difluorophen- yl)-2-(3,3-difluoropyr- rolidin-1-yl)pyridin-3- yl)pyrimidine-5-carbox- amide |
| 135 | 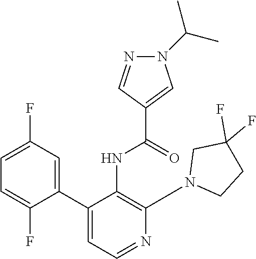 [SMILES: CC(C)n1cc(C(=O)Nc2c(-c3cc(F)ccc3F)ccnc2N2CCC(F)(F)C2)cn1] | N-(4-(2,5-difluorophen- yl)-2-(3,3-difluoropyr- rolidin-1-yl)pyridin-3- yl)-1-isopropyl-1H-pyr- azole-4-carboxamide |
| 136 | 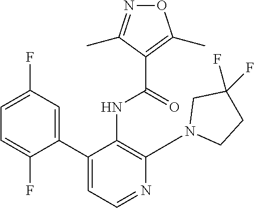 [SMILES: Cc1noc(C)c1C(=O)Nc1c(-c2cc(F)ccc2F)ccnc1N1CCC(F)(F)C1] | N-(4-(2,5-difluorophen- yl)-2-(3,3-difluoropyr- rolidin-1-yl)pyridin-3-yl)- 3,5-dimethylisoxazole- 4-carboxamide |
| 137 | 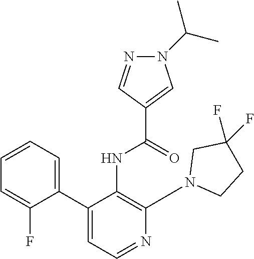 [SMILES: CC(C)n1cc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-1- isopropyl-1H-pyrazole- 4-carboxamide |
| 138 | 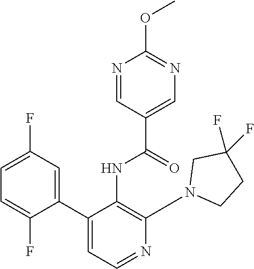 [SMILES: COc1ncc(C(=O)Nc2c(-c3cc(F)ccc3F)ccnc2N2CCC(F)(F)C2)cn1] | N-(4-(2,5-difluorophen- yl)-2-(3,3-difluoropyr- rolidin-1-yl)pyridin-3- yl)-2-methoxypyrimi- dine-5-carboxamide |
| 139 | 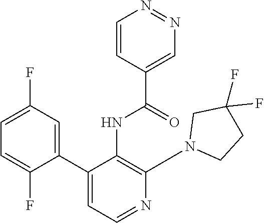 [SMILES: O=C(Nc1c(-c2cc(F)ccc2F)ccnc1N1CCC(F)(F)C1)c1ccnnc1] | N-(4-(2,5-difluorophen- yl)-2-(3,3-difluoropyr- rolidin-1-yl)pyridin-3- yl)pyridazine-4-carbox- amide |
| 140 | 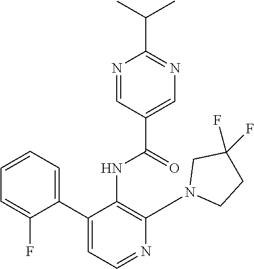 [SMILES: CC(C)c1ncc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-2- isopropylpyrimidine-5- carboxamide |
| 141 | 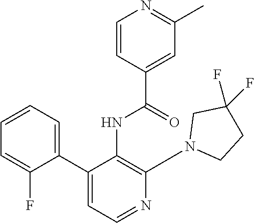 [SMILES: Cc1cc(C(=O)Nc2c(-c3ccccc3F)ccnc2N2CCC(F)(F)C2)ccn1] | N-(2-(3,3-difluoropyr- rolidin-1-yl)-4-(2-fluoro- phenyl)pyridin-3-yl)-2- methylisonicotinamide |
| 142 | 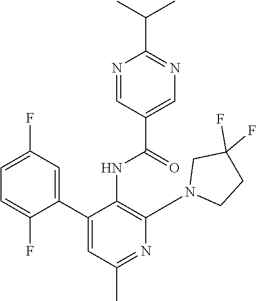 [SMILES: Cc1cc(-c2cc(F)ccc2F)c(NC(=O)c2cnc(C(C)C)nc2)c(N2CCC(F)(F)C2)n1] | N-(4-(2,5-difluorophen- yl)-2-(3,3-difluoropyr- rolidin-1-yl)-6-methyl- pyridin-3-yl)-2-isoprop- ylpyrimidine-5-carbox- amide |
| 143 | 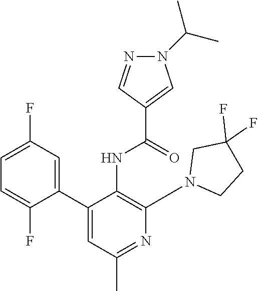 [SMILES: Cc1cc(-c2cc(F)ccc2F)c(NC(=O)c2cnn(C(C)C)c2)c(N2CCC(F)(F)C2)n1] | N-(4-(2,5-difluorophen- yl)-2-(3,3-difluoropyr- rolidin-1-yl)-6-methyl- pyridin-3-yl)-1-isoprop- yl-1H-pyrazole-4-carbox- amide |
| 144 | 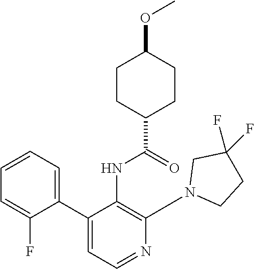 | (1r,4r)-N-(2-(3,3- difluoropyrrolidin-1-yl)- 4-(2-fluorophenyl)pyri- din-3-yl)-4-methoxy- cyclohexane-1-carbox- amide |
| 145 | 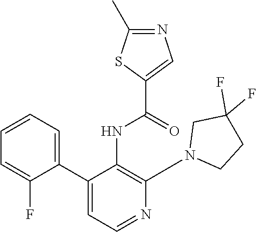 [SMILES: Cc1ncc(C(=O)Nc2c(-c3ccccc3F)ccnc2N2CCC(F)(F)C2)s1] | N-(2-(3,3-difluoropyr- rolidin-1-yl)-4-(2-fluoro- phenyl)pyridin-3-yl)-2- methylthiazole-5-carbox- amide |
| 146 | 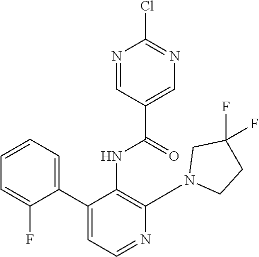 [SMILES: O=C(Nc1c(-c2ccccc2F)ccnc1N1CCC(F)(F)C1)c1cnc(Cl)nc1] | 2-chloro-N-(2-(3,3- difluoropyrrolidin-1-yl)- 4-(2-fluorophenyl)pyri- din-3-yl)pyrimidine-5- carboxamide |
| 147 | 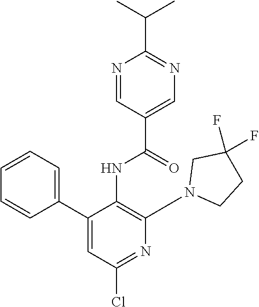 [SMILES: CC(C)c1ncc(C(=O)Nc2c(-c3ccccc3)cc(Cl)nc2N2CCC(F)(F)C2)cn1] | N-(6-chloro-2-(3,3- difluoro-pyrrolidin-1-yl)- 4-phenylpyridin-3-yl)-2- isopropylpyrimidine-5- carboxamide |
| 148 | 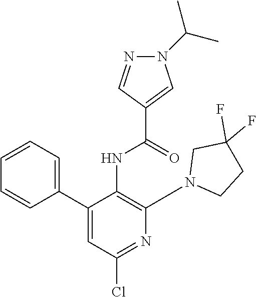 [SMILES: CC(C)n1cc(C(=O)Nc2c(-c3ccccc3)cc(Cl)nc2N2CCC(F)(F)C2)cn1] | N-(6-chloro-2-(3,3- difluoropyrrolidin-1-yl)- 4-phenylpyridin-3-yl)-1- isopropyl-1H-pyrazole-4- carboxamide |
| 149 | 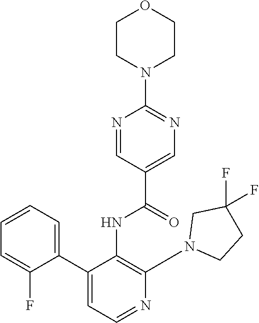 [SMILES: O=C(Nc1c(-c2ccccc2F)ccnc1N1CCC(F)(F)C1)c1cnc(N2CCOCC2)nc1] | N-(2-(3,3-difluoropyr- rolidin-1-yl)-4-(2-fluoro- phenyl)pyridin-3-yl)-2- morpholinopyrimidine- 5-carboxamide |
| 150 | 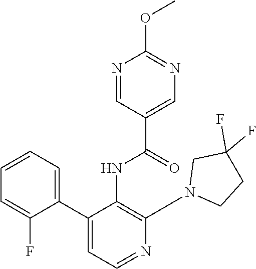 [SMILES: COc1ncc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-2- methoxypyrimidine-5- carboxamide |
| 151 | 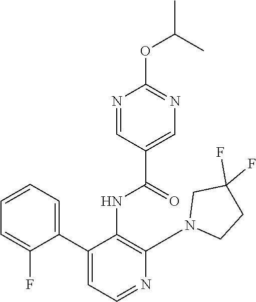 [SMILES: CC(C)Oc1ncc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-2- isopropoxypyrimidine- 5-carboxamide |
| 152 | 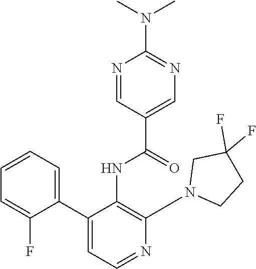 [SMILES: CN(C)c1ncc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-2- (dimethylamino)pyrimi- dine-5-carboxamide |
| 153 | 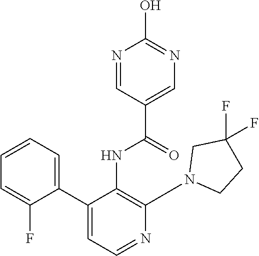 [SMILES: O=C(Nc1c(-c2ccccc2F)ccnc1N1CCC(F)(F)C1)c1cnc(O)nc1] | N-(2-(3,3-difluoropyr- rolidin-1-yl)-4-(2-fluoro- phenyl)pyridin-3-yl)-2- hydroxypyrimidine-5- carboxamide |
| 154 | 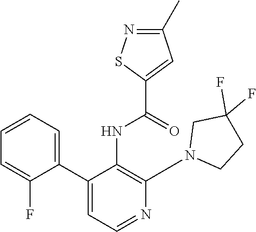 [SMILES: Cc1cc(C(=O)Nc2c(-c3ccccc3F)ccnc2N2CCC(F)(F)C2)sn1] | N-(2-(3,3-difluoropyr- rolidin-1-yl)-4-(2-fluoro- phenyl)pyridin-3-yl)-3- methylisothiazole-5- carboxamide |
| 155 | 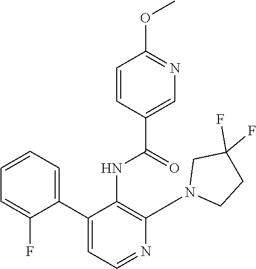 [SMILES: COc1ccc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-6- methoxynicotinamide |
| 156 | 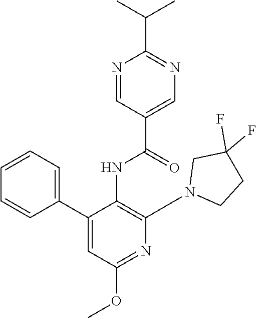 [SMILES: COc1cc(-c2ccccc2)c(NC(=O)c2cnc(C(C)C)nc2)c(N2CCC(F)(F)C2)n1] | N-(2-(3,3-difluoropyr- rolidin-1-yl)-6-methoxy- 4-phenylpyridin-3-yl)-2- isopropylpyrimidine-5- carboxamide |
| 157 | 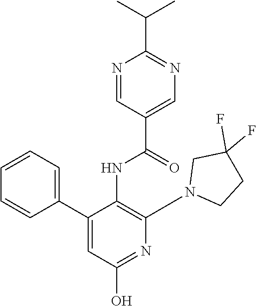 [SMILES: CC(C)c1ncc(C(=O)Nc2c(-c3ccccc3)cc(O)nc2N2CCC(F)(F)C2)cn1] | N-(2-(3,3-difluoropyr- rolidin-1-yl)-6-hydroxy- 4-phenylpyridin-3-yl)-2- isopropylpyrimidine-5- carboxamide |
| 158 | 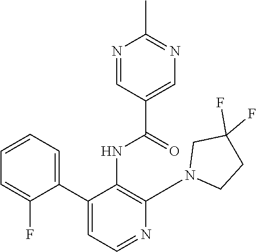 [SMILES: Cc1ncc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-2- methylpyrimidine-5- carboxamide |
| 159 | 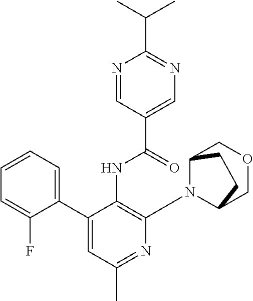 [SMILES: Cc1cc(-c2ccccc2F)c(NC(=O)c2cnc(C(C)C)nc2)c(N2[C@@H]3CC[C@H]2COC3)n1] | N-(2-(3-oxa-8-azabicyclo [3.2.1]octan-8-yl)-4-(2- fluorophenyl)pyridin-3- yl)-2-isopropylpyrimi- dine-5-carboxamide |
| 160 | 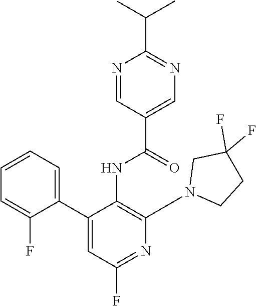 [SMILES: CC(C)c1ncc(C(=O)Nc2c(-c3ccccc3F)cc(F)nc2N2CCC(F)(F)C2)cn1] | N-(2-(3,3-difluoropyr- rolidin-1-yl)-6-fluoro-4- (2-fluorophenyl)pyridin- 3-yl)-2-isopropylpyrimi- dine-5-carboxamide |
| 161 | 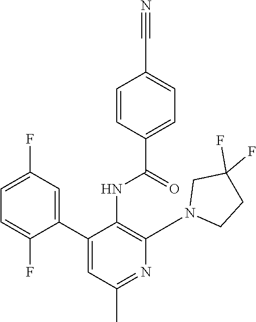 [SMILES: Cc1cc(-c2cc(F)ccc2F)c(NC(=O)c2ccc(C#N)cc2)c(N2CCC(F)(F)C2)n1] | 4-cyano-N-(4-(2,5- difluorophenyl)-2-(3,3- difluoropyrrolidin-1-yl)- 6-methylpyridin-3-yl)- benzamide |
| 162 | 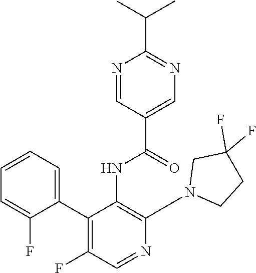 [SMILES: CC(C)c1ncc(C(=O)Nc2c(N3CCC(F)(F)C3)ncc(F)c2-c2ccccc2F)cn1] | N-(2-(3,3-difluoropyr- rolidin-1-yl)-5-fluoro-4- (2-fluorophenyl)pyridin- 3-yl)-2-isopropylpyrimi- dine-5-carboxamide |
| 163 | 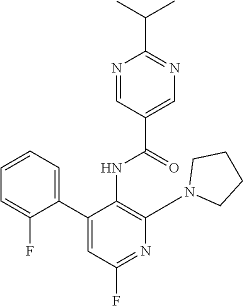 [SMILES: CC(C)c1ncc(C(=O)Nc2c(-c3ccccc3F)cc(F)nc2N2CCCC2)cn1] | N-(6-fluoro-4-(2-fluoro- phenyl)-2-(pyrrolidin-1- yl)pyridin-3-yl)-2-iso- propylpyrimidine-5- carboxamide |
| 164 | 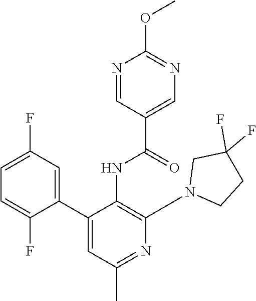 [SMILES: COc1ncc(C(=O)Nc2c(-c3cc(F)ccc3F)cc(C)nc2N2CCC(F)(F)C2)cn1] | N-(2-(3,3-difluoropyr- rolidin-1-yl)-4-(2-fluoro- phenyl)-6-methylpyridin- 3-yl)-2-methoxypyrimi- dine-5-carboxamide |
| 165 | 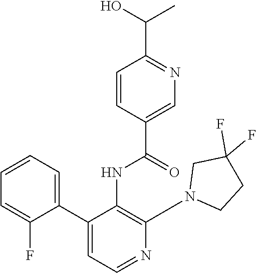 [SMILES: CC(O)c1ccc(C(=O)Nc2c(-c3ccccc3F)ccnc2N2CCC(F)(F)C2)cn1] | N-[2-(3,3-difluoropyr- rolidin-1-yl)-4-(2-fluoro- phenyl)-3-pyridyl]-6-(1- hydroxyethyl)pyridine-3- carboxamide |
| 166 | 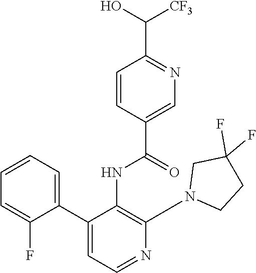 [SMILES: O=C(Nc1c(-c2ccccc2F)ccnc1N1CCC(F)(F)C1)c1ccc(C(O)C(F)(F)F)nc1] | N-[2-(3,3-difluoropyr- rolidin-1-yl)-4-(2-fluoro- phenyl)-3-pyridyl]-6- (2,2,2-trifluoro-1- hydroxy-ethyl)pyridine- 3-carboxamide |
| 167 | 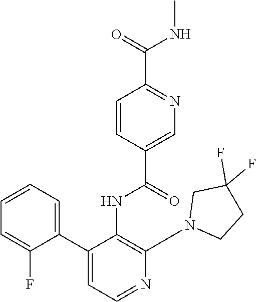 [SMILES: CNC(=O)c1ccc(C(=O)Nc2c(-c3ccccc3F)ccnc2N2CCC(F)(F)C2)cn1] | N-5-[2-(3,3-difluoropyr- rolidin-1-yl)-4-(2-fluoro- phenyl)-3-pyridyl]-N2- methyl-pyridine-2,5- dicarboxamide |
| 168 | 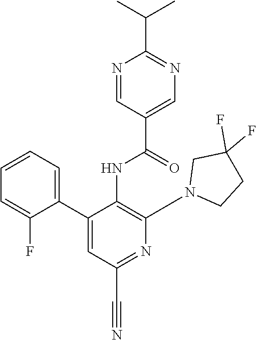 [SMILES: CC(C)c1ncc(C(=O)Nc2c(-c3ccccc3F)cc(C#N)nc2N2CCC(F)(F)C2)cn1] | N-[6-cyano-2-(3,3- difluoropyrrolidin-1-yl)- 4-(2-fluorophenyl)-3- pyridyl]-2-isopropyl- pyrimidine-5-carbox- amide |
| 169 | 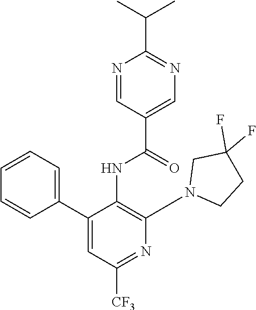 [SMILES: CC(C)c1ncc(C(=O)Nc2c(-c3ccccc3)cc(C(F)(F)F)nc2N2CCC(F)(F)C2)cn1] | N-[2-(3,3-difluoropyr- rolidin-1-yl)-4-phenyl-6- (trifluoromethyl)-3-pyr- idyl]-2-isopropyl-pyrimi- dine-5-carboxamide |
| 170 | 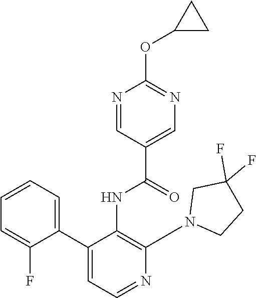 [SMILES: O=C(Nc1c(-c2ccccc2F)ccnc1N1CCC(F)(F)C1)c1cnc(OC2CC2)nc1] | 2-(cyclopropoxy)-N-[2- (3,3-difluoropyrrolidin- 1-yl)-4-(2-fluorophenyl)- 3-pyridyl]pyrimidine-5- carboxamide |
| 171 | 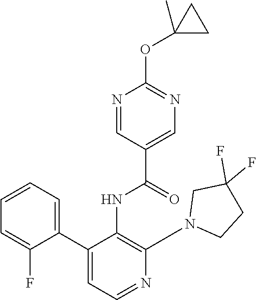 [SMILES: CC1(Oc2ncc(C(=O)Nc3c(-c4ccccc4F)ccnc3N3CCC(F)(F)C3)cn2)CC1] | N-[2-(3,3-difluoropyr- rolidin-1-yl)-4-(2-fluoro- phenyl)-3-pyridyl]-2-(1- methylcyclopropoxy)pyr- imidine-5-carboxamide |
| 172 | 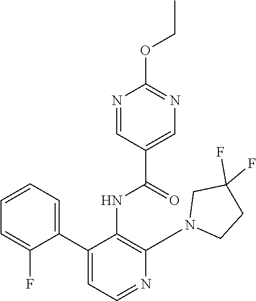 [SMILES: CCOc1ncc(C(=O)Nc2c(-c3ccccc3F)ccnc2N2CCC(F)(F)C2)cn1] | N-[2-(3,3-difluoropyr- rolidin-1-yl)-4-(2-fluoro- phenyl)-3-pyridyl]-2- ethoxy-pyrimidine-5- carboxamide |
| 173 | 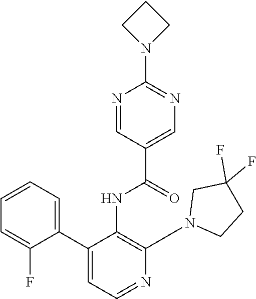 [SMILES: O=C(Nc1c(-c2ccccc2F)ccnc1N1CCC(F)(F)C1)c1cnc(N2CCC2)nc1] | 2-(azetidin-1-yl)-N-[2- (3,3-difluoropyrrolidin- 1-yl)-4-(2-fluorophenyl)- 3-pyridyl]pyrimidine-5- carboxamide |
| 174 | 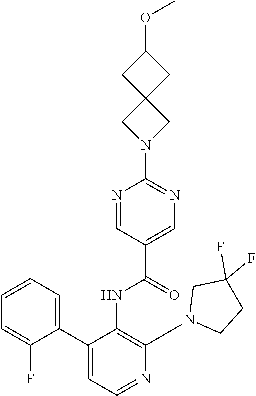 [SMILES: COC1CC2(C1)CN(c1ncc(C(=O)Nc3c(-c4ccccc4F)ccnc3N3CCC(F)(F)C3)cn1)C2] | N-[2-(3,3-difluoropyr- rolidin-1-yl)-4-(2-fluoro- phenyl)-3-pyridyl]-2-(6- methoxy-2-azaspiro[3.3] heptan-2-yl)pyrimidine- 5-carboxamide |
| 175 | 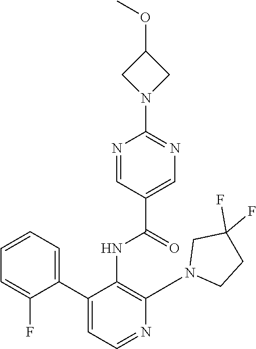 [SMILES: COC1CN(c2ncc(C(=O)Nc3c(-c4ccccc4F)ccnc3N3CCC(F)(F)C3)cn2)C1] | N-[2-(3,3-difluoropyr- rolidin-1-yl)-4-(2-fluoro- phenyl)-3-pyridyl]-2-(3- methoxyazetidin-1-yl)- pyrimidine-5-carbox- amide |
| 176 | 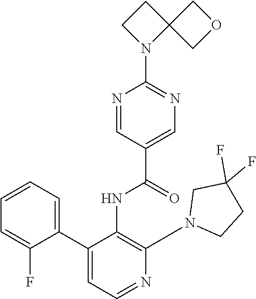 [SMILES: O=C(Nc1c(-c2ccccc2F)ccnc1N1CCC(F)(F)C1)c1cnc(N2CCC23COC3)nc1] | N-[2-(3,3-difluoropyr- rolidin-1-yl)-4-(2-fluoro- phenyl)-3-pyridyl]-2-(6- oxa-1-azaspiro[3.3] heptan-1-yl)pyrimidine- 5-carboxamide |
| 177 | 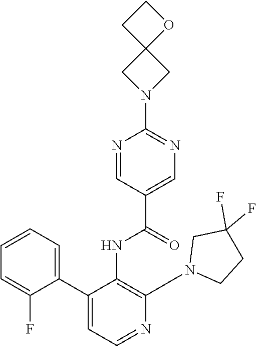 [SMILES: O=C(Nc1c(-c2ccccc2F)ccnc1N1CCC(F)(F)C1)c1cnc(N2CC3(CCO3)C2)nc1] | N-[2-(3,3-difluoropyrrol- idin-1-yl)-4-(2-fluoro- phenyl)-3-pyridyl]-2-(1- oxa-6-azaspiro[3.3] heptan-6-yl)pyrimidine- 5-carboxamide |
| 178 | 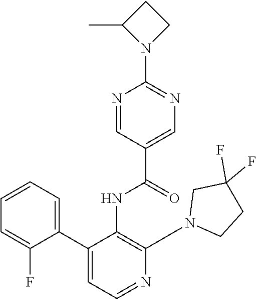 [SMILES: CC1CCN1c1ncc(C(=O)Nc2c(-c3ccccc3F)ccnc2N2CCC(F)(F)C2)cn1] | N-[2-(3,3-difluoropyr- rolidin-1-yl)-4-(2-fluoro- phenyl)-3-pyridyl]-2-(2- methylazetidin- 1-yl)pyrimidine-5- carboxamide |
| 179 | 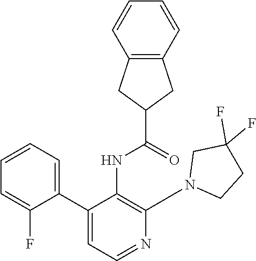 [SMILES: O=C(Nc1c(-c2ccccc2F)ccnc1N1CCC(F)(F)C1)C1Cc2ccccc2C1] | N-[2-(3,3-difluoropyr- rolidin-1-yl)-4-(2-fluoro- phenyl)-3-pyridyl] indane-2-carboxamide |
| 180 | 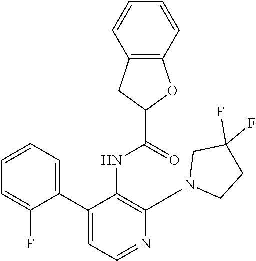 [SMILES: O=C(Nc1c(-c2ccccc2F)ccnc1N1CCC(F)(F)C1)C1Cc2ccccc2O1] | N-[2-(3,3-difluoropyr- rolidin-1-yl)-4-(2-fluoro- phenyl)-3-pyridyl]-2,3- dihydrobenzofuran-2- carboxamide |
| 181 | 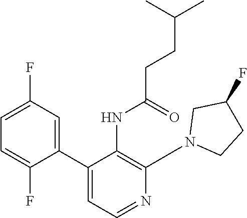 [SMILES: CC(C)CCC(=O)Nc1c(-c2cc(F)ccc2F)ccnc1N1CC[C@H](F)C1] | (S)-N-(4-(2,5-difluoro- phenyl)-2-(3-fluoro- pyrrolidin-1-yl)pyridin-3- yl)-4-methylpentanamide |
| 182 | 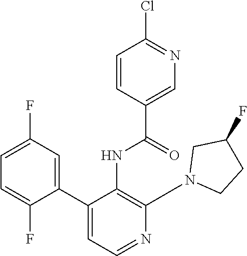 [SMILES: O=C(Nc1c(-c2cc(F)ccc2F)ccnc1N1CC[C@H](F)C1)c1ccc(Cl)nc1] | (S)-6-chloro-N-(4-(2,5- difluorophenyl)-2-(3- fluoropyrrolidin-1-yl)- pyridin-3-yl)nicotinamide |
| 183 | 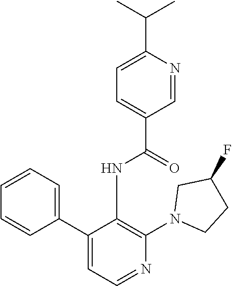 [SMILES: CC(C)c1ccc(C(=O)Nc2c(-c3ccccc3)ccnc2N2CC[C@H](F)C2)cn1] | (S)-N-(2-(3-fluoropyr- rolidin-1-yl)-4-phenyl- pyridin-3-yl)-6-isoprop- ylnicotinamide |
| 184 | 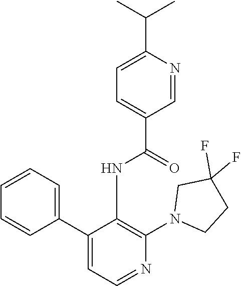 [SMILES: CC(C)c1ccc(C(=O)Nc2c(-c3ccccc3)ccnc2N2CCC(F)(F)C2)cn1] | N-(2-(3,3-difluoropyr- rolidin-1-yl)-4-phenyl- pyridin-3-yl)-6-isoprop- ylnicotinamide |
| 185 | 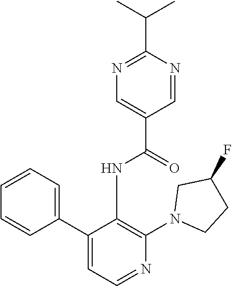 [SMILES: CC(C)c1ncc(C(=O)Nc2c(-c3ccccc3)ccnc2N2CC[C@H](F)C2)cn1] | (S)-N-(2-(3-fluoropyr- rolidin-1-yl)-4-phenyl- pyridin-3-yl)-2-isoprop- ylpyrimidine-5-carbox- amide |
| 186 | 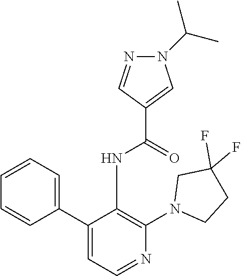 [SMILES: CC(C)n1cc(C(=O)Nc2c(-c3ccccc3)ccnc2N2CCC(F)(F)C2)cn1] | N-(2-(3,3-difluoropyr- rolidin-1-yl)-4-phenyl- pyridin-3-yl)-1-isoprop- yl-1H-pyrazole-4- carboxamide |
| 187 | 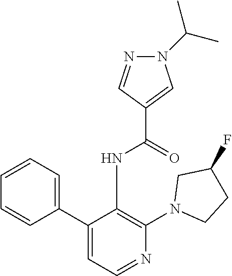 [SMILES: CC(C)n1cc(C(=O)Nc2c(-c3ccccc3)ccnc2N2CC[C@H](F)C2)cn1] | (S)-N-(2-(3-fluoropyr- rolidin-1-yl)-4-phenyl- pyridin-3-yl)-1-isoprop- yl-1H-pyrazole-4- carboxamide |
| 188 | 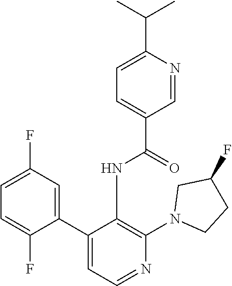 [SMILES: CC(C)c1ccc(C(=O)Nc2c(-c3cc(F)ccc3F)ccnc2N2CC[C@H](F)C2)cn1] | (S)-N-(4-(2,5-difluoro- phenyl)-2-(3-fluoropyr- rolidin-1-yl)pyridin-3- yl)-6-isopropylnicotin- amide |
| 189 | 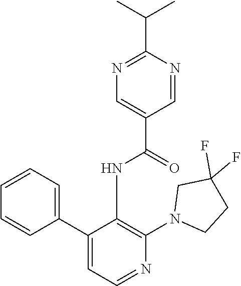 [SMILES: CC(C)c1ncc(C(=O)Nc2c(-c3ccccc3)ccnc2N2CCC(F)(F)C2)cn1] | N-(2-(3,3-difluoropyr- rolidin-1-yl)-4-phenyl- pyridin-3-yl)-2-isoprop- ylpyrimidine-5-carbox- amide |
| 190 | 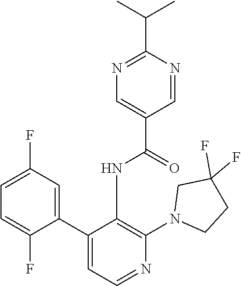 [SMILES: CC(C)c1ncc(C(=O)Nc2c(-c3cc(F)ccc3F)ccnc2N2CCC(F)(F)C2)cn1] | N-(4-(2,5-difluorophen- yl)-2-(3,3-difluoropyr- rolidin-1-yl)pyridin-3- yl)-2-isopropylpyrimi- dine-5-carboxamide |
| 191 | 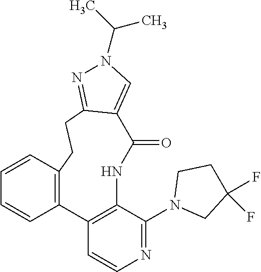 [SMILES: CC(C)n1cc2c(n1)CCc1ccccc1-c1ccnc(N3CCC(F)(F)C3)c1NC2=O] | 6-(3,3-difluoropyrroli- din-1-yl)-12-isopropyl- 5, 8, 12, 13-tetrazatetra- cyclo[15.4.0.02, 7.010, 14]henicosa-1(21),2(7), 3, 5, 10, 13, 17, 19- octaen-9-one |
| 192 | 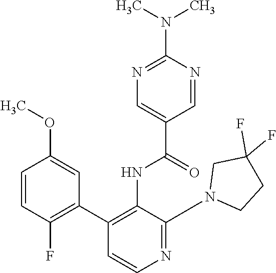 [SMILES: COc1ccc(F)c(-c2ccnc(N3CCC(F)(F)C3)c2NC(=O)c2cnc(N(C)C)nc2)c1] | N-[2-(3,3-difluoropyr- rolidin-1-yl)-4-(2-fluoro- 5-methoxy-phenyl)-3- pyridyl]-2-(dimethyl- amino)pyrimidine-5- carboxamide |
| 193 | 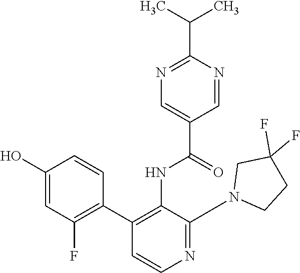 [SMILES: CC(C)c1ncc(C(=O)Nc2c(-c3ccc(O)cc3F)ccnc2N2CCC(F)(F)C2)cn1] | N-[2-(3,3-difluoropyrrol- idin-1-yl)-4-(2-fluoro-4- hydroxy-phenyl)-3-pyr- idyl]-2-isopropyl-pyrimi- dine-5-carboxamide |
| 194 | 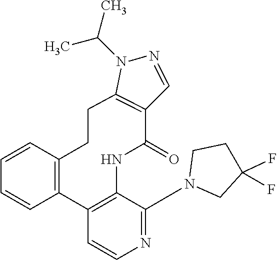 [SMILES: CC(C)n1ncc2c1CCc1ccccc1-c1ccnc(N3CCC(F)(F)C3)c1NC2=O] | 6-(3,3-difluoropyrrol- idin-1-yl)-13-isopropyl- 5, 8, 12, 13-tetrazatetra- cyclo[15.4.0.02, 7.010, 14]henicosa-1(21), 2(7), 3, 5, 10(14), 11, 17, 19- octaen-9-one |
| 195 | 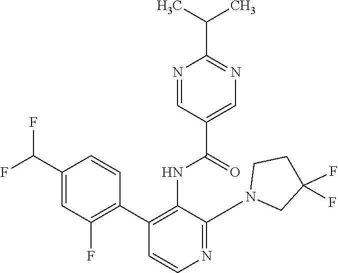 [SMILES: CC(C)c1ncc(C(=O)Nc2c(-c3ccc(C(F)F)cc3F)ccnc2N2CCC(F)(F)C2)cn1] | N-[4-[4-(difluorometh- yl)-2-fluoro-phenyl]-2- (3,3-difluoropyrrolidin- 1-yl)-3-pyridyl]-2-iso- propyl-pyrimidine-5- carboxamide |
| 196 | 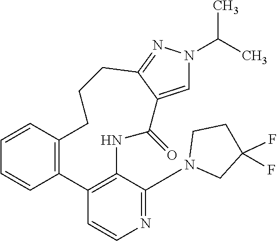 [SMILES: CC(C)n1cc2c(n1)CCCc1ccccc1-c1ccnc(N3CCC(F)(F)C3)c1NC2=O] | 6-(3,3-difluoropyrrol- idin-1-yl)-12-isopropyl- 5,8,12,13-tetrazatetra- cyclo[16.4.0.02, 7.010, 14]docosa-1(22), 2(7), 3, 5, 10, 13, 18, 20- octaen-9-one |
| 197 | 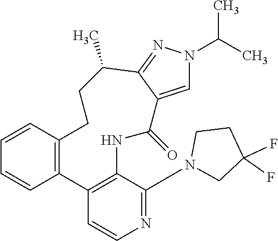 [SMILES: CC(C)n1cc2c(n1)[C@@H](C)CCc1ccccc1-c1ccnc(N3CCC(F)(F)C3)c1NC2=O] | (15R)-6-(3,3-difluoro- pyrrolidin-1-yl)-12-iso- propyl-15-methyl-5, 8, 12,13-tetrazatetracyclo [15.4.0.02, 7.010, 14] henicosa-1(21), 2(7), 3, 5, 10, 13, 17, 19-octaen- 9-one |
| 198 | 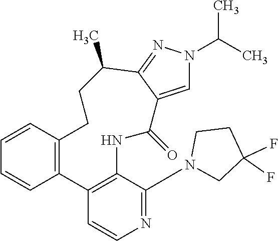 [SMILES: CC(C)n1cc2c(n1)[C@H](C)CCc1ccccc1-c1ccnc(N3CCC(F)(F)C3)c1NC2=O] | (15S)-6-(3,3-difluoro- pyrrolidin-1-yl)-12-iso- propyl-15-methyl-5, 8, 12, 13-tetrazatetracyclo [15.4.0.02, 7.010, 14] henicosa-1(21), 2(7), 3, 5, 10, 13, 17, 19-octaen- 9 one |
| 199 | 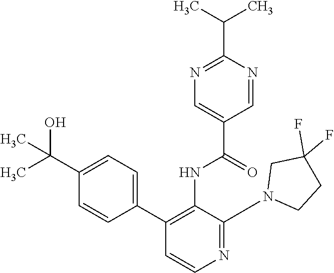 [SMILES: CC(C)c1ncc(C(=O)Nc2c(-c3ccc(C(C)(C)O)cc3)ccnc2N2CCC(F)(F)C2)cn1] | N-[2-(3,3-difluoropyr- rolidin-1-yl)-4-[4-(1- hydroxy-1-methyl-ethyl)- phenyl]-3-pyridyl]-2- isopropyl-pyrimidine-5- carboxamide |
| 200 | 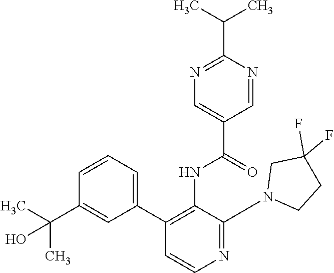 [SMILES: CC(C)c1ncc(C(=O)Nc2c(-c3cccc(C(C)(C)O)c3)ccnc2N2CCC(F)(F)C2)cn1] | N-[2-(3,3-difluoropyr- rolidin-1-yl)-4-[3-(1- hydroxy-1-methyl-ethyl)- phenyl]-3-pyridyl]-2- isopropyl-pyrimidine-5- carboxamide |
| 201 | 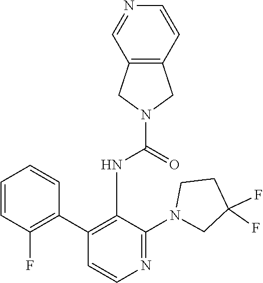 [SMILES: O=C(Nc1c(-c2ccccc2F)ccnc1N1CCC(F)(F)C1)N1Cc2ccncc2C1] | N-[2-(3,3-difluoropyr- rolidin-1-yl)-4-(2-fluoro- phenyl)-3-pyridyl]-1,3- dihydropyrrolo[3,4-c] pyridine-2-carboxamide |
| 202 | 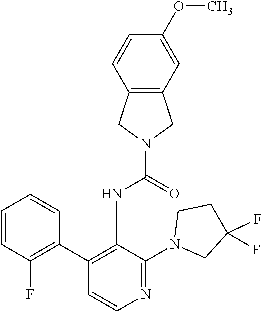 [SMILES: COc1ccc2c(c1)CN(C(=O)Nc1c(-c3ccccc3F)ccnc1N1CCC(F)(F)C1)C2] | N-[2-(3,3-difluoropyr- rolidin-1-yl)-4-(2-fluoro- phenyl)-3-pyridyl]-5- methoxy-isoindoline-2- carboxamide |
| 203 | 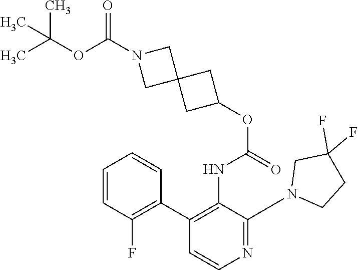 [SMILES: CC(C)(C)OC(=O)N1CC2(CC(OC(=O)Nc3c(-c4ccccc4F)ccnc3N3CCC(F)(F)C3)C2)C1] | tert-butyl 6-[2-(3,3- difluoropyrrolidin-1-yl)- 4-(2-fluorophenyl)-3- pyridyl]carbamoyloxy]- 2-azaspiro[3.3]heptane- 2-carboxylate |
| 204 | 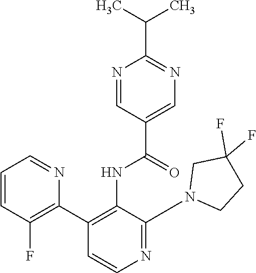 [SMILES: CC(C)c1ncc(C(=O)Nc2c(-c3ncccc3F)ccnc2N2CCC(F)(F)C2)cn1] | N-[2-(3,3-difluoropyr- rolidin-1-yl)-4-(3-fluoro- 2-pyridyl)-3-pyridyl]-2- isopropyl-pyrimidine-5- carboxamide |
| 205 | 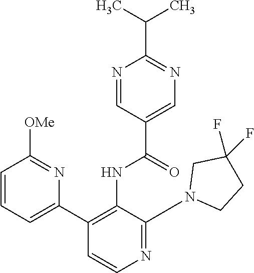 [SMILES: COc1cccc(-c2ccnc(N3CCC(F)(F)C3)c2NC(=O)c2cnc(C(C)C)nc2)n1] | N-[2-(3,3-difluoropyr- rolidin-1-yl)-4-(6-meth- oxy-2-pyridyl)-3-pyr- idyl]-2-isopropyl-pyr- imidine-5-carboxamide |
| 206 | 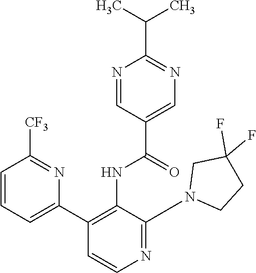 [SMILES: CC(C)c1ncc(C(=O)Nc2c(-c3cccc(C(F)(F)F)n3)ccnc2N2CCC(F)(F)C2)cn1] | N-[2-(3,3-difluoropyr- rolidin-1-yl)-4-[6- (trifluoromethyl)-2- pyridyl]-3-pyridyl]-2- isopropyl-pyrimidine- 5-carboxamide |
| 207 | 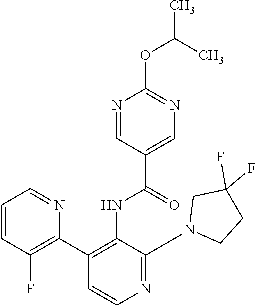 [SMILES: CC(C)Oc1ncc(C(=O)Nc2c(-c3ncccc3F)ccnc2N2CCC(F)(F)C2)cn1] | N-[2-(3,3-difluoropyr- rolidin-1-yl)-4-(3-fluoro- 2-pyridyl)-3-pyridyl]-2- isopropoxy-pyrimidine- 5-carboxamide |
| 208 | 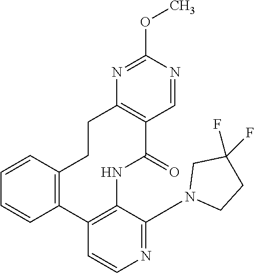 [SMILES: COc1ncc2c(n1)CCc1ccccc1-c1ccnc(N3CCC(F)(F)C3)c1NC2=O] | 6-(3,3-difluoropyrroli- din-1-yl)-13-methoxy- 5, 8, 12, 14-tetrazatetra- cyclo[16.4.0.02, 7.010, 15]docosa-1(22), 2(7), 3, 5, 10(15), 11, 13, 18, 20-nonaen-9-one |
| 209 | 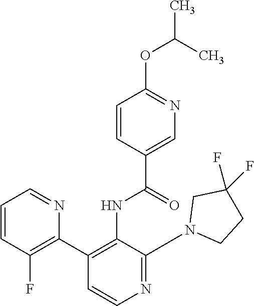 [SMILES: CC(C)Oc1ccc(C(=O)Nc2c(-c3ncccc3F)ccnc2N2CCC(F)(F)C2)cn1] | N-[2-(3,3-difluoropyrroli- din-1-yl)-4-(3-fluoro-2- pyridyl)-3-pyridyl]-6- isopropoxy-pyridine-3- carboxamide |
| 210 | 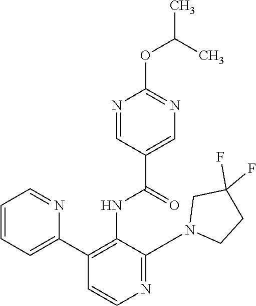 [SMILES: CC(C)Oc1ncc(C(=O)Nc2c(-c3ccccn3)ccnc2N2CCC(F)(F)C2)cn1] | N-[2-(3,3-difluoropyr- rolidin-1-yl)-4-(2-pyrid- yl)-3-pyridyl]-2-isopro- poxy-pyrimidine-5- carboxamide |
| 211 | 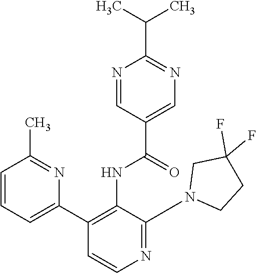 [SMILES: Cc1cccc(-c2ccnc(N3CCC(F)(F)C3)c2NC(=O)c2cnc(C(C)C)nc2)n1] | N-[2-(3,3-difluoropyr- rolidin-1-yl)-4-(6-meth- yl-2-pyridyl)-3-pyridyl]- 2-isopropyl-pyrimidine- 5-carboxamide |
| 212 | 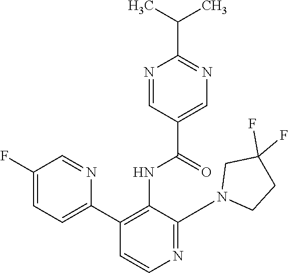 [SMILES: CC(C)c1ncc(C(=O)Nc2c(-c3ccc(F)cn3)ccnc2N2CCC(F)(F)C2)cn1] | N-[2-(3,3-difluoropyr- rolidin-1-yl)-4-(5-fluoro- 2-pyridyl)-3-pyridyl]-2- isopropyl-pyrimidine-5- carboxamide |
| 213 | 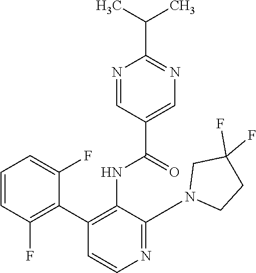 [SMILES: CC(C)c1ncc(C(=O)Nc2c(-c3c(F)cccc3F)ccnc2N2CCC(F)(F)C2)cn1] | N-[4-(2,6-difluorophen- yl)-2-(3,3-difluoropyr- rolidin-1-yl)-3-pyridyl]- 2-isopropyl-pyrimidine- 5-carboxamide |
| 214 | 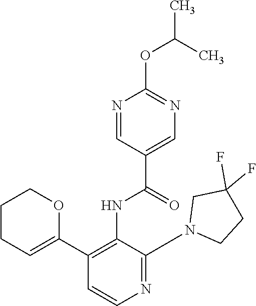 [SMILES: CC(C)Oc1ncc(C(=O)Nc2c(C3=CCCCO3)ccnc2N2CCC(F)(F)C2)cn1] | N-[2-(3,3-difluoropyr- rolidin-1-yl)-4-(3,4- dihydro-2H-pyran-6- yl)-3-pyridyl]-2-iso- propoxy-pyrimidine-5- carboxamide |
| 215 | 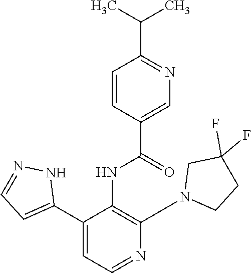 [SMILES: CC(C)c1ccc(C(=O)Nc2c(-c3ccn[nH]3)ccnc2N2CCC(F)(F)C2)cn1] | N-(2-(3,3-difluoropyr- rolidin-1-yl)-4-(1H- pyrazol-5-yl)pyridin-3- yl)-6-isopropylnicotin- amide |
| 216 | 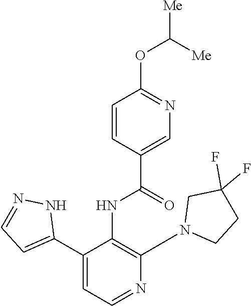 [SMILES: CC(C)Oc1ccc(C(=O)Nc2c(-c3ccn[nH]3)ccnc2N2CCC(F)(F)C2)cn1] | N-(2-(3,3-difluoropyr- rolidin-1-yl)-4-(1H- pyrazol-5-yl)pyridin-3- yl)-6-isopropoxynicotin- amide |
| 217 | 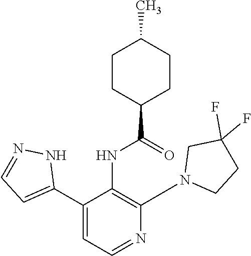 [SMILES: C[C@H]1CC[C@H](C(=O)Nc2c(-c3ccn[nH]3)ccnc2N2CCC(F)(F)C2)CC1] | (1r,4r)-N-(2-(3,3-difluoro- pyrrolidin-1-yl)-4-(1H- pyrazol-5-yl)pyridin-3- yl)-4-methylcyclohexane- 1-carboxamide |
| 218 | 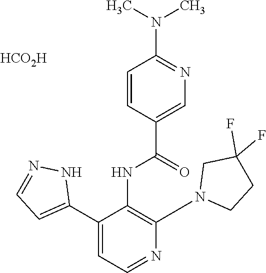 [SMILES: "HCO2H" ; CN(C)c1ccc(C(=O)Nc2c(-c3ccn[nH]3)ccnc2N2CCC(F)(F)C2)cn1] | N-(2-(3,3-difluoropyr- rolidin-1-yl)-4-(1H- pyrazol-5-yl)pyridin-3- yl)-6-(dimethylamino)- nicotinamide formate salt |
| 219 | 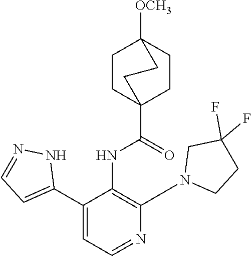 [SMILES: COC12CCC(C(=O)Nc3c(-c4ccn[nH]4)ccnc3N3CCC(F)(F)C3)(CC1)CC2] | N-(2-(3,3-difluoropyr- rolidin-1-yl)-4-(1H-pyr- azol-5-yl)pyridin-3-yl)- 4-methoxybicyclo[2.2.2] octane-1-carboxamide |
| 220 | 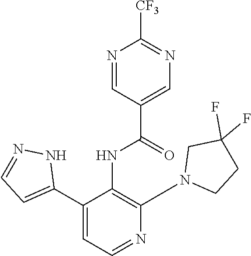 [SMILES: O=C(Nc1c(-c2ccn[nH]2)ccnc1N1CCC(F)(F)C1)c1cnc(C(F)(F)F)nc1] | N-(2-(3,3-difluoropyr- rolidin-1-yl)-4-(1H-pyr- azol-5-yl)pyridin-3-yl)- 2-(trifluoromethyl)pyr- imidine-5-carboxamide |
| 221 | 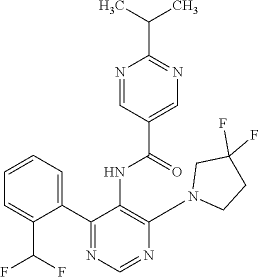 [SMILES: CC(C)c1ncc(C(=O)Nc2c(-c3ccccc3C(F)F)ncnc2N2CCC(F)(F)C2)cn1] | N-(4-(2-(difluoromethyl)- phenyl)-6-(3,3-difluoro- pyrrolidin-1-yl)-pyrimi- din-5-yl)-2-isopropyl- pyrimidine-5-carbox- amide |
| 222 | 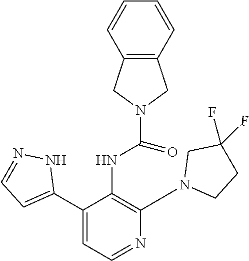 [SMILES: O=C(Nc1c(-c2ccn[nH]2)ccnc1N1CCC(F)(F)C1)N1Cc2ccccc2C1] | N-(2-(3,3-difluoropyr- rolidin-1-yl)-4-(1H-pyr- azol-5-yl)pyridin-3-yl)- isoindoline-2-carbox- amide |
| 223 | 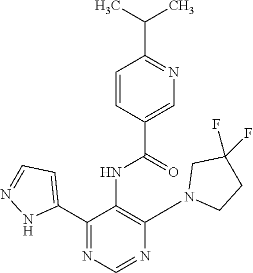 [SMILES: CC(C)c1ccc(C(=O)Nc2c(-c3ccn[nH]3)ncnc2N2CCC(F)(F)C2)cn1] | N-(4-(3,3-difluoropyr- rolidin-1-yl)-6-(1H-pyr- azol-5-yl)pyrimidin-5- yl)-6-isopropylnicotin- amide |
| 224 | 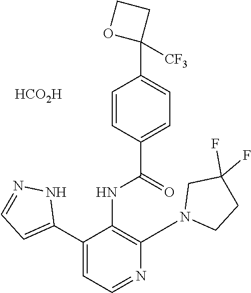 [SMILES: "HCO2H" ; O=C(Nc1c(-c2ccn[nH]2)ccnc1N1CCC(F)(F)C1)c1ccc(C2(C(F)(F)F)CCO2)cc1] | N-(2-(3,3-difluoropyr- rolidin-1-yl)-4-(1H- indazol-5-yl)pyridin-3- yl)-2-isopropylpyrimi- dine-5-carboxamide formate salt |
| 225 | 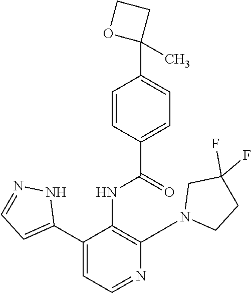 [SMILES: CC1(c2ccc(C(=O)Nc3c(-c4ccn[nH]4)ccnc3N3CCC(F)(F)C3)cc2)CCO1] | N-(2-(3,3-difluoropyr- rolidin-1-yl)-4-(1H-pyr- azol-5-yl)pyridin-3-yl)- 4-(2-methyloxetan-2-yl)- benzamide |
| 226 | 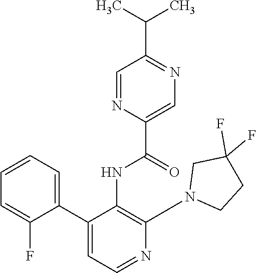 [SMILES: CC(C)c1cnc(C(=O)Nc2c(-c3ccccc3F)ccnc2N2CCC(F)(F)C2)cn1] | N-(2-(3,3-difluoropyrrol- idin-1-yl)-4-(2-fluoro- phenyl)pyridin-3-yl)-5- isopropylpyrazine-2- carboxamide |
| 227 | 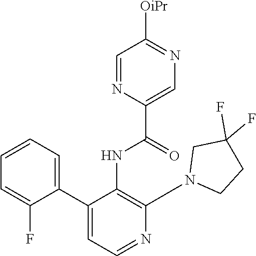 [SMILES: CC(C)Oc1cnc(C(=O)Nc2c(-c3ccccc3F)ccnc2N2CCC(F)(F)C2)cn1] | N-(2-(3,3-difluoropyrrol- idin-1-yl)-4-(2-fluoro- phenyl)pyridin-3-yl)-5- isopropoxypyrazine-2- carboxamide |
| 228 | 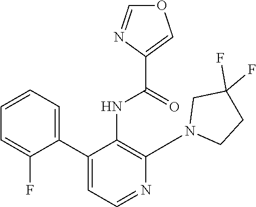 [SMILES: O=C(Nc1c(-c2ccccc2F)ccnc1N1CCC(F)(F)C1)c1cocn1] | N-(2-(3,3-difluoropyrrol- idin-1-yl)-4-(2-fluoro- phenyl)pyridin-3-yl)- oxazole-4-carboxamide |
| 229 | 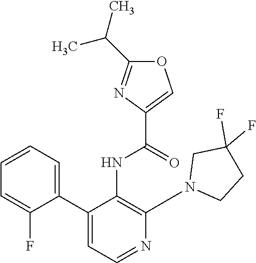 [SMILES: CC(C)c1nc(C(=O)Nc2c(-c3ccccc3F)ccnc2N2CCC(F)(F)C2)co1] | N-(2-(3,3-difluoropyrrol- idin-1-yl)-4-(2-fluoro- phenyl)pyridin-3-yl)-2- isopropyloxazole-4- carboxamide |
| 230 | 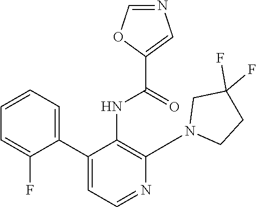 [SMILES: O=C(Nc1c(-c2ccccc2F)ccnc1N1CCC(F)(F)C1)c1cnco1] | N-(2-(3,3-difluoropyrrol- idin-1-yl)-4-(2-fluoro- phenyl)pyridin-3-yl)- oxazole-5-carboxamide |
| 231 | 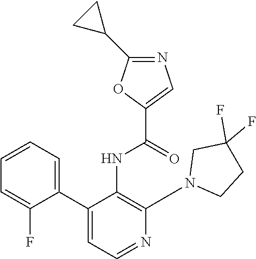 [SMILES: O=C(Nc1c(-c2ccccc2F)ccnc1N1CCC(F)(F)C1)c1cnc(C2CC2)o1] | 2-cyclopropyl-N-(2-(3,3- difluoropyrrolidin-1-yl)- 4-(2-fluorophenyl)pyr- idin-3-yl)oxazole-5- carboxamide |
| 232 | 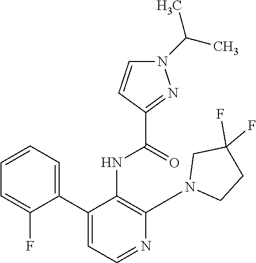 [SMILES: CC(C)n1ccc(C(=O)Nc2c(-c3ccccc3F)ccnc2N2CCC(F)(F)C2)n1] | N-(2-(3,3-difluoropyrrol- idin-1-yl)-4-(2-fluoro- phenyl)pyridin-3-yl)-1- isopropyl-1H-pyrazole- 3-carboxamide |
| 233 | 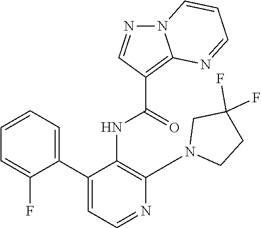 [SMILES: O=C(Nc1c(-c2ccccc2F)ccnc1N1CCC(F)(F)C1)c1cnn2cccnc12] | N-(2-(3,3-difluoropyr- rolidin-1-yl)-4-(2-fluoro- phenyl)pyridin-3-yl)pyr- azolo[1,5-a]pyrimidine- 3-carboxamide |
| 234 | 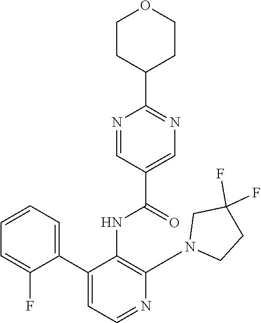 [SMILES: O=C(Nc1c(-c2ccccc2F)ccnc1N1CCC(F)(F)C1)c1cnc(C2CCOCC2)nc1] | N-(2-(3,3-difluoropyr- rolidin-1-yl)-4-(2-fluoro- phenyl)pyridin-3-yl)-2- (tetrahydro-2H-pyran-4- yl)pyrimidine-5-carbox- amide |
| 235 | 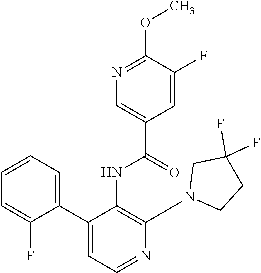 [SMILES: COc1ncc(C(=O)Nc2c(-c3ccccc3F)ccnc2N2CCC(F)(F)C2)cc1F] | N-(2-(3,3-difluoropyr- rolidin-1-yl)-4-(2-fluoro- phenyl)pyridin-3-yl)-5- fluoro-6-methoxynicotin- amide |
| 236 | 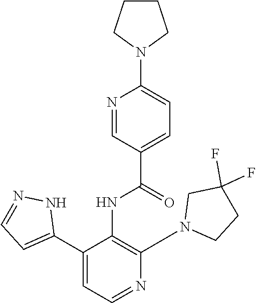 [SMILES: O=C(Nc1c(-c2ccn[nH]2)ccnc1N1CCC(F)(F)C1)c1ccc(N2CCCC2)nc1] | N-(2-(3,3-difluoropyr- rolidin-1-yl)-4-(1H-pyr- azol-5-yl)pyridin-3-yl)-6- (pyrrolidin-1-yl)nicotin- amide |
| 237 | 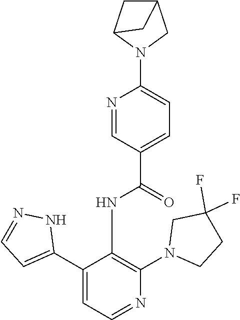 [SMILES: O=C(Nc1c(-c2ccn[nH]2)ccnc1N1CCC(F)(F)C1)c1ccc(N2CC3CC2C3)nc1] | 6-(2-azabicyclo[2.1.1] hexan-2-yl)-N-(2-(3,3- difluoropyrrolidin-1-yl)- 4-(1H-pyrazol-5-yl)pyri- din-3-yl)nicotinamide |
| 238 | 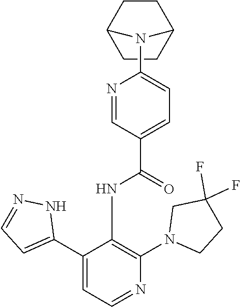 [SMILES: O=C(Nc1c(-c2ccn[nH]2)ccnc1N1CCC(F)(F)C1)c1ccc(N2C3CCC2CC3)nc1] | 6-(7-azabicyclo[2.2.1] heptan-7-yl)-N-(2-(3,3- difluoropyrrolidin-1-yl)- 4-(1H-pyrazol-5-yl)pyri- din-3-yl)nicotinamide |
| 239 | 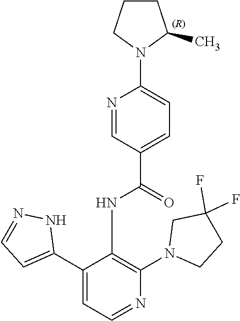 | (R)-N-(2-(3,3-difluoro- pyrrolidin-1-yl)-4-(1H- pyrazol-5-yl)pyridin-3- yl)-6-(2-methylpyrroli- din-1-yl)nicotinamide |
| 240 | 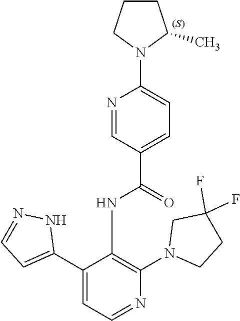 | (S)-N-(2-(3,3-difluoro- pyrrolidin-1-yl)-4-(1H- pyrazol-5-yl)pyridin-3- yl)-6-(2-methylpyrroli- din-1-yl)nicotinamide |
| 241 | 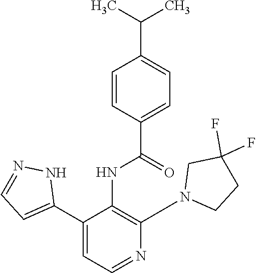 [SMILES: CC(C)c1ccc(C(=O)Nc2c(-c3ccn[nH]3)ccnc2N2CCC(F)(F)C2)cc1] | N-(2-(3,3-difluoropyr- rolidin-1-yl)-4-(1H-pyr- azol-5-yl)pyridin-3-yl)- 4-isopropylbenzamide |
| 242 | 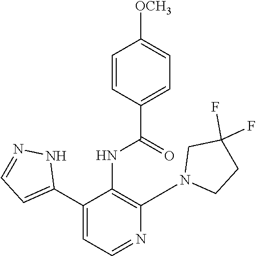 [SMILES: COc1ccc(C(=O)Nc2c(-c3ccn[nH]3)ccnc2N2CCC(F)(F)C2)cc1] | N-(2-(3,3-difluoropyr- rolidin-1-yl)-4-(1H-pyr- azol-5-yl)pyridin-3-yl)- 4-methoxybenzamide |
| 243 | 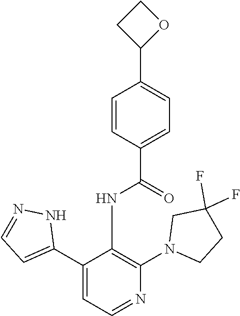 [SMILES: O=C(Nc1c(-c2ccn[nH]2)ccnc1N1CCC(F)(F)C1)c1ccc(C2CCO2)cc1] | N-(2-(3,3-difluoropyr- rolidin-1-yl)-4-(1H-pyr- azol-5-yl)pyridin-3-yl)- 4-(oxetan-2-yl)benz- amide |
| 244 | 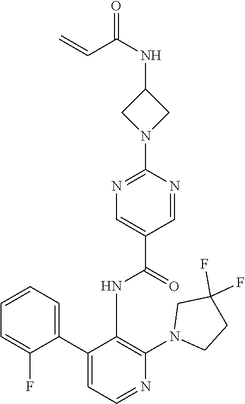 [SMILES: C=CC(=O)NC1CN(c2ncc(C(=O)Nc3c(-c4ccccc4F)ccnc3N3CCC(F)(F)C3)cn2)C1] | 2-(3-acrylamidoazetidin- 1-yl)-N-(2-(3,3-difluoro- pyrrolidin-1-yl)-4-(2- fluorophenyl)pyridin-3- yl)pyrimidine-5-carbox- amide |
| 245 | 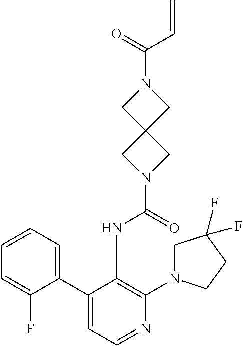 [SMILES: C=CC(=O)N1CC2(C1)CN(C(=O)Nc1c(-c3ccccc3F)ccnc1N1CCC(F)(F)C1)C2] | 6-acryloyl-N-(2-(3,3- difluoropyrrolidin-1-yl)- 4-(2-fluorophenyl)pyr- idin-3-yl)-2,6-diazaspiro [3.3]heptane-2-carbox- amide |
| 246 | 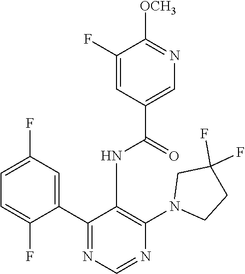 [SMILES: COc1ncc(C(=O)Nc2c(-c3cc(F)ccc3F)ncnc2N2CCC(F)(F)C2)cc1F] | N-(4-(2,5-difluorophen- yl)-6-(3,3-difluoropyr- rolidin-1-yl)pyrimidin-5- yl)-5-fluoro-6-methoxy- nicotinamide |
| 247 | 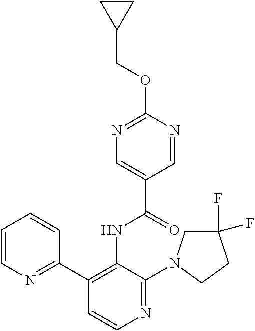 [SMILES: O=C(Nc1c(-c2ccccn2)ccnc1N1CCC(F)(F)C1)c1cnc(OCC2CC2)nc1] | 2-(cyclopropylmethoxy)- N-(2′-(3,3-difluoropyr- rolidin-1-yl)-[2,4′-bipyr- idin]-3′-yl)pyrimidine-5- carboxamide |
| 248 | 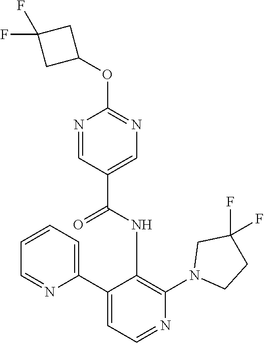 [SMILES: O=C(Nc1c(-c2ccccn2)ccnc1N1CCC(F)(F)C1)c1cnc(OC2CC(F)(F)C2)nc1] | 2-(3,3-difluorocyclobut- oxy)-N-(2′-(3,3-difluoro- pyrrolidin-1-yl)-[2,4′- bipyridin]-3′-yl)pyrimi- dine-5-carboxamide |
| 249 | 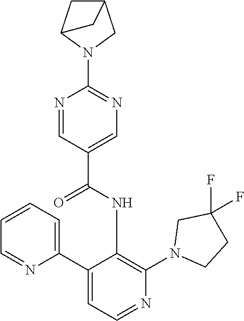 [SMILES: O=C(Nc1c(-c2ccccn2)ccnc1N1CCC(F)(F)C1)c1cnc(N2CC3CC2C3)nc1] | 2-(2-azabicyclo[2.1.1] hexan-2-yl)-N-(2′-(3,3- difluoropyrrolidin-1-yl)- [2,4′-bipyridin]-3′-yl)- pyrimidine-5-carboxamide |
| 250 | 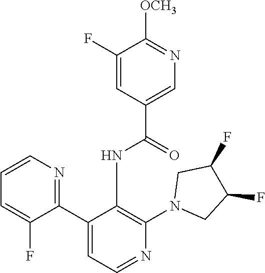 [SMILES: COc1ncc(C(=O)Nc2c(-c3ncccc3F)ccnc2N2C[C@@H](F)[C@@H](F)C2)cc1F] | N-(2′-((3S,4R)-3,4- difluoropyrrolidin-1-yl)- 3-fluoro-[2,4′-bipyridin]- 3′-yl)-5-fluoro-6-meth- oxynicotinamide |
| 251 | 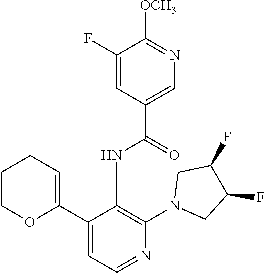 [SMILES: COc1ncc(C(=O)Nc2c(C3=CCCCO3)ccnc2N2C[C@@H](F)[C@@H](F)C2)cc1F] | N-(2-((3S,4R)-3,4- difluoropyrrolidin-1-yl)- 4-(3,4-dihydro-2H-pyran- 6-yl)pyridin-3-yl)-5- fluoro-6-methoxynicotin- amide |
| 252 | 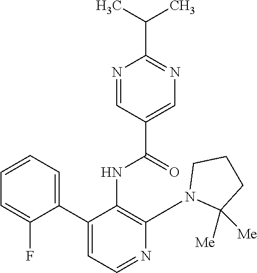 [SMILES: CC(C)c1ncc(C(=O)Nc2c(-c3ccccc3F)ccnc2N2CCCC2(C)C)cn1] | N-(2-(2,2-dimethylpyr- rolidin-1-yl)-4-(o-tolyl)- pyridin-3-yl)-2-isoprop- ylpyrimidine-5-carbox- amide |
| 253 | 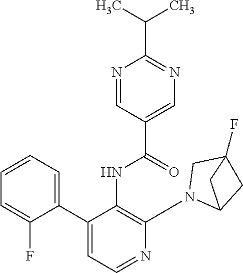 [SMILES: CC(C)c1ncc(C(=O)Nc2c(-c3ccccc3F)ccnc2N2CC3(F)CC2C3)cn1] | N-(2-(4-fluoro-2-aza- bicyclo[2.1.1]hexan-2- yl)-4-(2-fluorophenyl)- pyridin-3-yl)-2-isoprop- ylpyrimidine-5-carbox- amide |
| 254 | 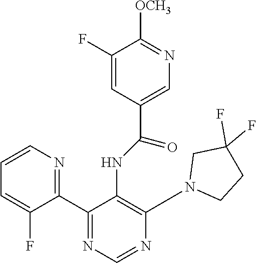 [SMILES: COc1ncc(C(=O)Nc2c(-c3ncccc3F)ncnc2N2CCC(F)(F)C2)cc1F] | N-(4-(3,3-difluoropyr- rolidin-1-yl)-6-(3-fluoro- pyridin-2-yl)pyrimidin- 5-yl)-5-fluoro-6-meth- oxynicotinamide |
| 255 | 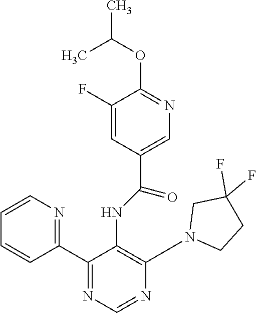 [SMILES: CC(C)Oc1ncc(C(=O)Nc2c(-c3ccccn3)ncnc2N2CCC(F)(F)C2)cc1F] | N-(4-(3,3-difluoropyr- rolidin-1-yl)-6-(3-fluoro- pyridin-2-yl)pyrimidin- 5-yl)-5-fluoro-6-meth- oxynicotinamide |
| 256 | 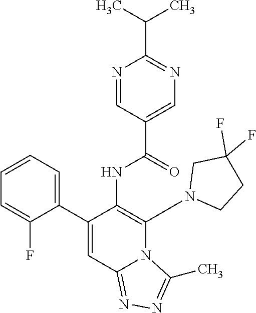 [SMILES: Cc1nnc2cc(-c3ccccc3F)c(NC(=O)c3cnc(C(C)C)nc3)c(N3CCC(F)(F)C3)n12] | N-(5-(3,3-difluoropyr- rolidin-1-yl)-7-(2-fluoro- phenyl)-3-methyl-[1,2,4] triazolo[4,3-a]pyridin-6- yl)-2-isopropylpyrimi- dine-5-carboxamide |
| 257 | 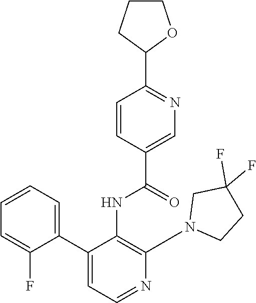 [SMILES: O=C(Nc1c(-c2ccccc2F)ccnc1N1CCC(F)(F)C1)c1ccc(C2CCCO2)nc1] | N-(2-(3,3-difluoropyr- rolidin-1-yl)-4-(2-fluoro- phenyl)pyridin-3-yl)-6- (tetrahydrofuran-2-yl)- nicotinamide |
| 258 | 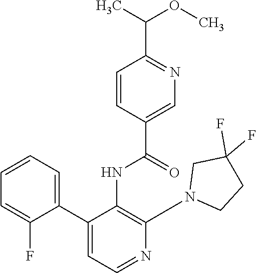 [SMILES: COC(C)c1ccc(C(=O)Nc2c(-c3ccccc3F)ccnc2N2CCC(F)(F)C2)cn1] | N-(2-(3,3-difluoropyr- rolidin-1-yl)-4-(2-fluoro- phenyl)pyridin-3-yl)-6- (1-methoxyethyl)nicotin- amide |
| 259 | 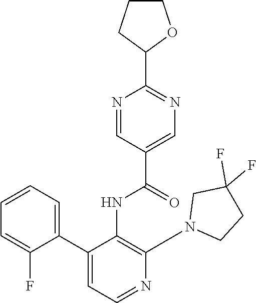 [SMILES: O=C(Nc1c(-c2ccccc2F)ccnc1N1CCC(F)(F)C1)c1cnc(C2CCCO2)nc1] | N-(2-(3,3-difluoropyr- rolidin-1-yl)-4-(2-fluoro- phenyl)pyridin-3-yl)-2- (tetrahydrofuran-2-yl)- pyrimidine-5-carbox- amide |
| 260 | 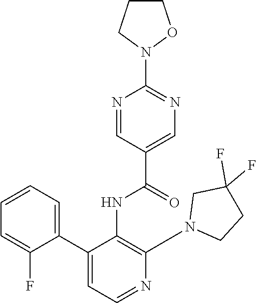 [SMILES: O=C(Nc1c(-c2ccccc2F)ccnc1N1CCC(F)(F)C1)c1cnc(N2CCCO2)nc1] | N-(2-(3,3-difluoropyr- rolidin-1-yl)-4-(2-fluoro- phenyl)pyridin-3-yl)-2- (isoxazolidin-2-yl)pyr- imidine-5-carboxamide |
| 261 | 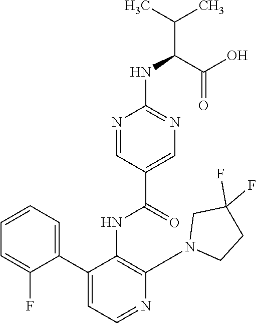 [SMILES: CC(C)[C@H](Nc1ncc(C(=O)Nc2c(-c3ccccc3F)ccnc2N2CCC(F)(F)C2)cn1)C(=O)O] | (5-((2-(3,3-difluoropyr- rolidin-1-yl)-4-(2-fluoro- phenyl)pyridin-3-yl)car- bamoyl)pyrimidin-2-yl)- L-valine |
| 262 | 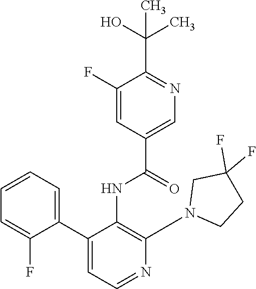 [SMILES: CC(C)(O)c1ncc(C(=O)Nc2c(-c3ccccc3F)ccnc2N2CCC(F)(F)C2)cc1F] | N-[2-(3,3-difluoropyr- rolidin-1-yl)-4-(2-fluoro- phenyl)-3-pyridyl]-5- fluoro-6-(1-hydroxy-1- methyl-ethyl)pyridine- 3-carboxamide |
| 263 | 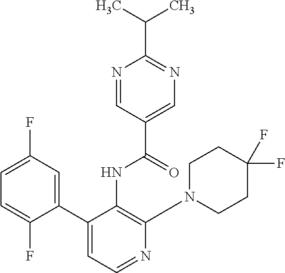 [SMILES: CC(C)c1ncc(C(=O)Nc2c(-c3cc(F)ccc3F)ccnc2N2CCC(F)(F)CC2)cn1] | N-[4-(2,5-difluorophen- yl)-2-(4,4-difluoro-1- piperidyl)-3-pyridyl]-2- isopropyl-pyrimidine-5- carboxamide |
| 264 | 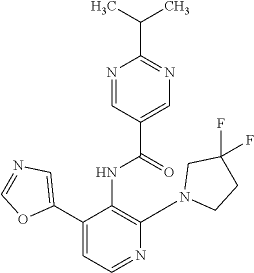 [SMILES: CC(C)c1ncc(C(=O)Nc2c(-c3cnco3)ccnc2N2CCC(F)(F)C2)cn1] | N-(2-(3,3-difluoropyr- rolidin-1-yl)-4-(oxazol- 5-yl)pyridin-3-yl)-2- isopropylpyrimidine-5- carboxamide |
| 265 | 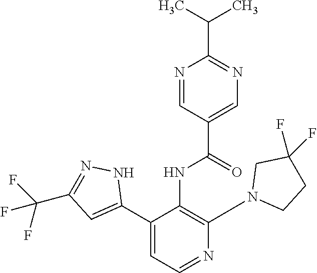 [SMILES: CC(C)c1ncc(C(=O)Nc2c(-c3cc(C(F)(F)F)n[nH]3)ccnc2N2CCC(F)(F)C2)cn1] | N-(2-(3,3-difluoropyr- rolidin-1-yl)-4-(3-(tri- fluoromethyl)-1H-pyr- azol-5-yl)pyridin-3-yl)- 2-isopropylpyrimidine- 5-carboxamide |
| 266 | 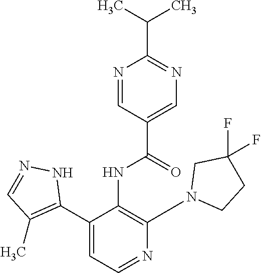 [SMILES: Cc1cn[nH]c1-c1ccnc(N2CCC(F)(F)C2)c1NC(=O)c1cnc(C(C)C)nc1] | N-[2-(3,3-difluoropyr- rolidin-1-yl)-4-(4-meth- yl-1H-pyrazol-5-yl)-3- pyridyl]-2-isopropyl- pyrimidine-5-carbox- amide |
| 267 | 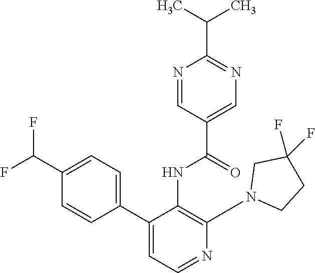 [SMILES: CC(C)c1ncc(C(=O)Nc2c(-c3ccc(C(F)F)cc3)ccnc2N2CCC(F)(F)C2)cn1] | N-[4-[4-(difluoromethyl)- phenyl]-2-(3,3-difluoro- pyrrolidin-1-yl)-3-pyrid- yl]-2-isopropyl-pyrimi- dine-5-carboxamide |
| 268 | 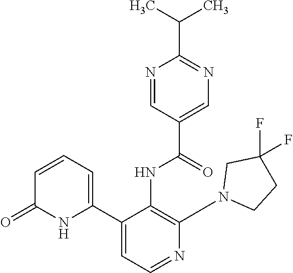 [SMILES: CC(C)c1ncc(C(=O)Nc2c(-c3cccc(=O)[nH]3)ccnc2N2CCC(F)(F)C2)cn1] | N-[2-(3,3-difluoropyr- rolidin-1-yl)-4-(6-oxo- 1H-pyridin-2-yl)-3-pyrid- yl]-2-isopropyl-pyrimi- dine-5-carboxamide |
| 269 | 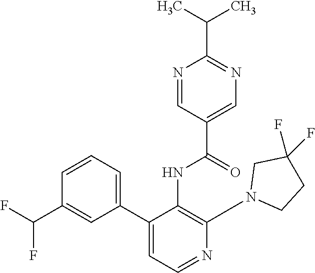 [SMILES: CC(C)c1ncc(C(=O)Nc2c(-c3cccc(C(F)F)c3)ccnc2N2CCC(F)(F)C2)cn1] | N-[4-[3-(difluoromethyl)- phenyl]-2-(3,3-difluoro- pyrrolidin-1-yl)-3-pyrid- yl]-2-isopropyl-pyrimi- dine-5-carboxamide |
| 270 | 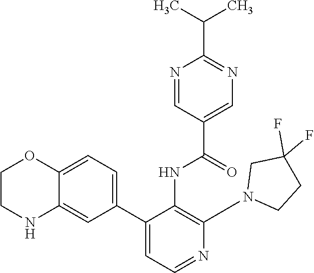 [SMILES: CC(C)c1ncc(C(=O)Nc2c(-c3ccc4c(c3)NCCO4)ccnc2N2CCC(F)(F)C2)cn1] | N-[2-(3,3-difluoropyr- rolidin-1-yl)-4-(3,4- dihydro-2H-1,4-benzox- azin-6-yl)-3-pyridyl]-2- isopropyl-pyrimidine-5- carboxamide |
| 271 | 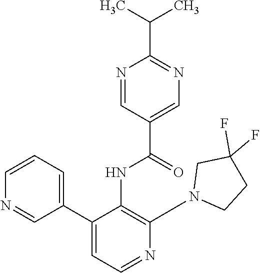 [SMILES: CC(C)c1ncc(C(=O)Nc2c(-c3cccnc3)ccnc2N2CCC(F)(F)C2)cn1] | N-[2-(3,3-difluoropyr- rolidin-1-yl)-4-(3-pyrid- yl)-3-pyridyl]-2-isoprop- yl-pyrimidine-5-carbox- amide |
| 272 | 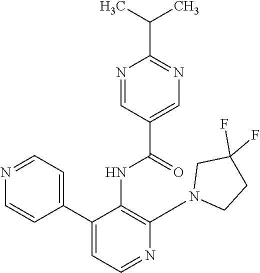 [SMILES: CC(C)c1ncc(C(=O)Nc2c(-c3ccncc3)ccnc2N2CCC(F)(F)C2)cn1] | N-[2-(3,3-difluoropyr- rolidin-1-yl)-4-(4-pyrid- yl)-3-pyridyl]-2-isoprop- yl-pyrimidine-5-carbox- amide |
| 273 | 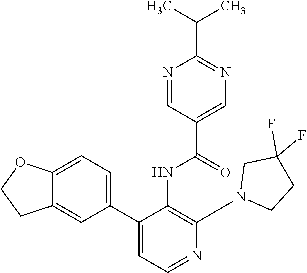 [SMILES: CC(C)c1ncc(C(=O)Nc2c(-c3ccc4c(c3)CCO4)ccnc2N2CCC(F)(F)C2)cn1] | N-(2-(3,3-difluoropyr- rolidin-1-yl)-4-(2,3- dihydrobenzofuran-5- yl)pyridin-3-yl)-2-iso- propylpyrimidine-5- carboxamide |
| 274 | 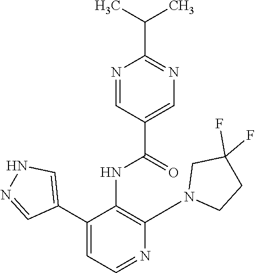 [SMILES: CC(C)c1ncc(C(=O)Nc2c(-c3cn[nH]c3)ccnc2N2CCC(F)(F)C2)cn1] | N-(2-(3,3-difluoropyr- rolidin-1-yl)-4-(1H-pyr- azol-4-yl)pyridin-3-yl)- 2-isopropylpyrimidine- 5-carboxamide |
| 275 | 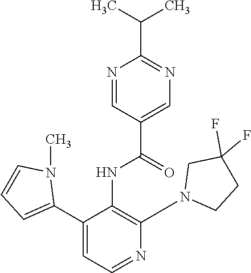 [SMILES: CC(C)c1ncc(C(=O)Nc2c(-c3cccn3C)ccnc2N2CCC(F)(F)C2)cn1] | N-(2-(3,3-difluoropyr- rolidin-1-yl)-4-(1-meth- yl-1H-pyrrol-2-yl)pyri- din-3-yl)-2-isopropyl- pyrimidine-5-carbox- amide |
| 276 | 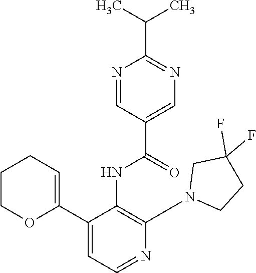 [SMILES: CC(C)c1ncc(C(=O)Nc2c(C3=CCCCO3)ccnc2N2CCC(F)(F)C2)cn1] | N-(2-(3,3-difluoropyr- rolidin-1-yl)-4-(3,4- dihydro-2H-pyran-6- yl)pyridin-3-yl)-2-iso- propylpyrimidine-5- carboxamide |
| 277 | 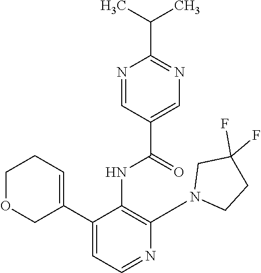 [SMILES: CC(C)c1ncc(C(=O)Nc2c(C3=CCCOC3)ccnc2N2CCC(F)(F)C2)cn1] | N-(2-(3,3-difluoropyr- rolidin-1-yl)-4-(3,6- dihydro-2H-pyran-5-yl)- pyridin-3-yl)-2-isoprop- ylpyrimidine-5-carbox- amide |
| 278 | 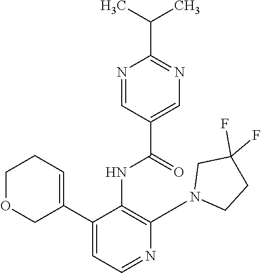 [SMILES: CC(C)c1ncc(C(=O)Nc2c(C3=CCCOC3)ccnc2N2CCC(F)(F)C2)cn1] | N-[4-cyclopropyl-2-(3,3- difluoropyrrolidin-1-yl)- 3-pyridyl]-2-isopropyl- pyrimidine-5-carbox- amide |
| 279 | 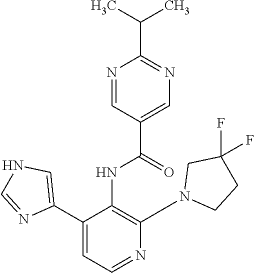 [SMILES: CC(C)c1ncc(C(=O)Nc2c(-c3c[nH]cn3)ccnc2N2CCC(F)(F)C2)cn1] | N-[2-(3,3-difluoropyr- rolidin-1-yl)-4-(1H-imid- azol-4-yl)-3-pyridyl]-2- isopropyl-pyrimidine-5- carboxamide |
| 280 | 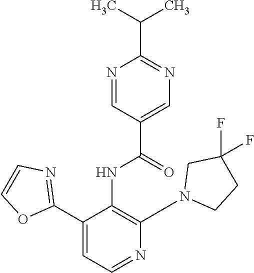 [SMILES: CC(C)c1ncc(C(=O)Nc2c(-c3ncco3)ccnc2N2CCC(F)(F)C2)cn1] | N-[2-(3,3-difluoropyr- rolidin-1-yl)-4-oxazol-2- yl-3-pyridyl]-2-isoprop- yl-pyrimidine-5-carbox- amide |
| 281 | 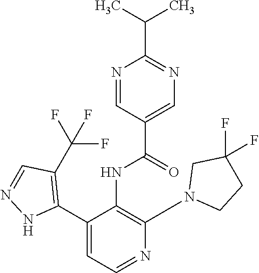 [SMILES: CC(C)c1ncc(C(=O)Nc2c(-c3[nH]ncc3C(F)(F)F)ccnc2N2CCC(F)(F)C2)cn1] | N-[2-(3,3-difluoropyr- rolidin-1-yl)-4-[4-(tri- fluoromethyl)-1H-pyr- azol-5-yl]-3-pyridyl]-2- isopropyl-pyrimidine-5- carboxamide |
| 282 | 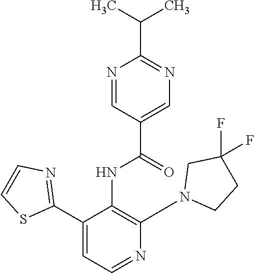 [SMILES: CC(C)c1ncc(C(=O)Nc2c(-c3nccs3)ccnc2N2CCC(F)(F)C2)cn1] | N-(2-(3,3-difluoropyr- rolidin-1-yl)-4-(thiazol- 2-yl)pyridin-3-yl)-2- isopropylpyrimidine-5- carboxamide |
| 283 | 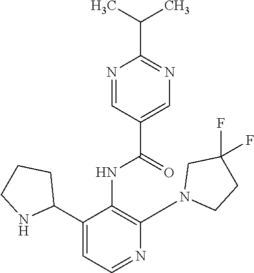 [SMILES: CC(C)c1ncc(C(=O)Nc2c(C3CCCN3)ccnc2N2CCC(F)(F)C2)cn1] | N-[2-(3,3-difluoropyr- rolidin-1-yl)-4-pyrroli- din-2-yl-3-pyridyl]-2- isopropyl-pyrimidine- 5-carboxamide |
| 284 | 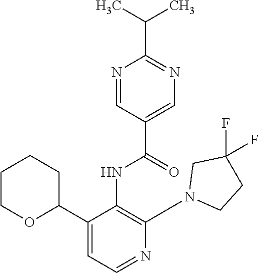 [SMILES: CC(C)c1ncc(C(=O)Nc2c(C3CCCCO3)ccnc2N2CCC(F)(F)C2)cn1] | N-[2-(3,3-difluoropyr- rolidin-1-yl)-4-tetra- hydropyran-2-yl-3- pyridyl]-2-isopropyl- pyrimidine-5-carbox- amide |
| 285 | 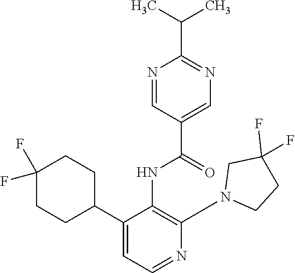 [SMILES: CC(C)c1ncc(C(=O)Nc2c(C3CCC(F)(F)CC3)ccnc2N2CCC(F)(F)C2)cn1] | N-[4-(4,4-difluorocyclo- hexyl)-2-(3,3-difluoro- pyrrolidin-1-yl)-3-pyrid- yl]-2-isopropyl-pyrimi- dine-5-carboxamide |
| 286 | 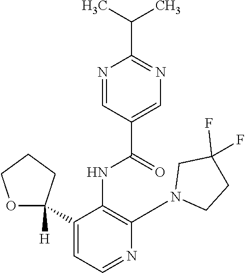 [SMILES: CC(C)c1ncc(C(=O)Nc2c([C@H]3CCCO3)ccnc2N2CCC(F)(F)C2)cn1] | N-[2-(3,3-difluoropyr- rolidin-1-yl)-4-[(2R)- tetrahydrofuran-2-yl]-3- pyridyl]-2-isopropyl-pyr- imidine-5-carboxamide |
| 287 | 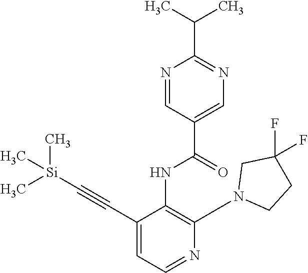 [SMILES: CC(C)c1ncc(C(=O)Nc2c(C#C[Si](C)(C)C)ccnc2N2CCC(F)(F)C2)cn1] | N-[2-(3,3-difluoropyr- rolidin-1-yl)-4-(2-tri- methylsilylethynyl)-3- pyridyl]-2-isopropyl-pyr- imidine-5-carboxamide |
| 288 | 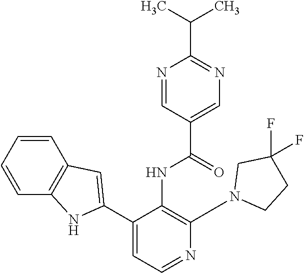 [SMILES: CC(C)c1ncc(C(=O)Nc2c(-c3cc4ccccc4[nH]3)ccnc2N2CCC(F)(F)C2)cn1] | N-[2-(3,3-difluoropyr- rolidin-1-yl)-4-(1H- indol-2-yl)-3-pyridyl]-2- isopropyl-pyrimidine-5- carboxamide |
| 289 | 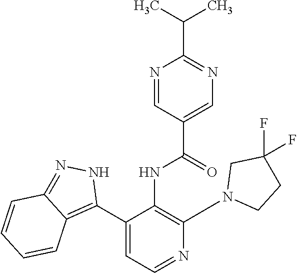 [SMILES: CC(C)c1ncc(C(=O)Nc2c(-c3[nH]nc4ccccc34)ccnc2N2CCC(F)(F)C2)cn1] | 2-(3,3-difluoropyrrol- idin-1-yl)-4-(2H-indazol- 3-yl)-3-pyridyl]-2-iso- propyl-pyrimidine-5- carboxamide |
| 290 | 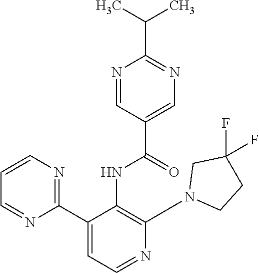 [SMILES: CC(C)c1ncc(C(=O)Nc2c(-c3ncccn3)ccnc2N2CCC(F)(F)C2)cn1] | N-[2-(3,3-difluoropyr- rolidin-1-yl)-4-pyrimi- din-2-yl-3-pyridyl]-2- isopropyl-pyrimidine-5- carboxamide |
| 291 | 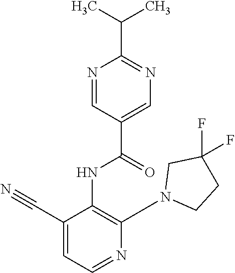 [SMILES: CC(C)c1ncc(C(=O)Nc2c(C#N)ccnc2N2CCC(F)(F)C2)cn1] | N-[4-cyano-2-(3,3- difluoropyrrolidin-1-yl)- 3-pyridyl]-2-isopropyl- pyrimidine-5-carbox- amide |
| 292 | 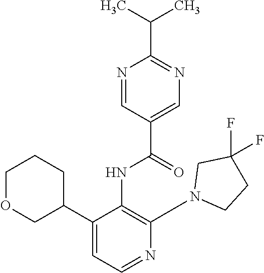 [SMILES: CC(C)c1ncc(C(=O)Nc2c(C3CCCOC3)ccnc2N2CCC(F)(F)C2)cn1] | N-[2-(3,3-difluoropyr- rolidin-1-yl)-4-tetra- hydropyran-3-yl-3- pyridyl]-2-isopropyl- pyrimidine-5-carbox- amide |
| 293 | 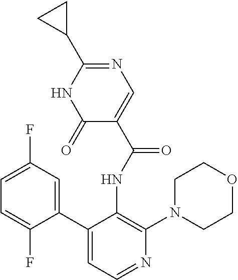 [SMILES: O=C(Nc1c(-c2cc(F)ccc2F)ccnc1N1CCOCC1)c1cnc(C2CC2)[nH]c1=O] | 2-cyclopropyl-N-(4- (2,5-difluorophenyl)-2- morpholinopyridin-3-yl)- 6-oxo-1,6-dihydropyr- imidine-5-carboxamide |
| 294 | 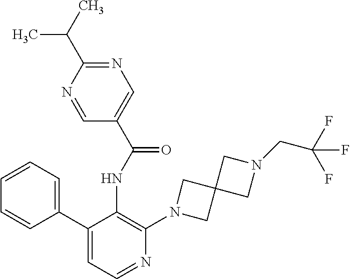 [SMILES: CC(C)c1ncc(C(=O)Nc2c(-c3ccccc3)ccnc2N2CC3(CN(CC(F)(F)F)C3)C2)cn1] | 2-Isopropyl-N-(4-phen- yl-2-(6-(2,2,2-trifluoro- ethyl)-2,6-diazaspiro [3.3]heptan-2-yl)pyridin- 3-yl)pyrimidine-5- carboxamide |
| 295 | 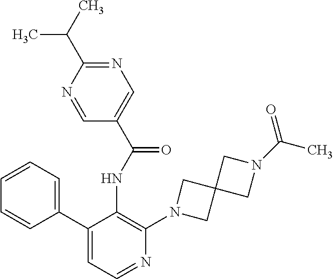 [SMILES: CC(=O)N1CC2(C1)CN(c1nccc(-c3ccccc3)c1NC(=O)c1cnc(C(C)C)nc1)C2] | N-(2-(6-acetyl-2,6-diaza- spiro[3.3]heptan-2-yl)- 4-phenylpyridin-3-yl)-2- isopropylpyrimidine-5- carboxamide |
| 296 | 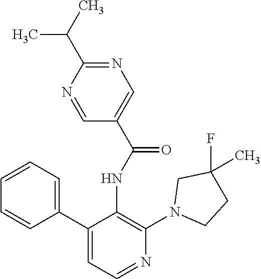 [SMILES: CC(C)c1ncc(C(=O)Nc2c(-c3ccccc3)ccnc2N2CCC(C)(F)C2)cn1] | N-(2-(3-fluoro-3-methyl- pyrrolidin-1-yl)-4-phen- ylpyridin-3-yl)-2-iso- propylpyrimidine-5- carboxamide |
| 297 | 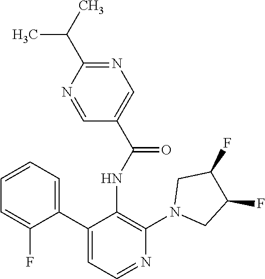 [SMILES: CC(C)c1ncc(C(=O)Nc2c(-c3ccccc3F)ccnc2N2C[C@@H](F)[C@@H](F)C2)cn1] | N-(2-((3S,4R)-3,4- difluoropyrrolidin-1-yl)- 4-(2-fluorophenyl)pyri- din-3-yl)-2-isopropylpyr- imidine-5-carboxamide |
| 298 | 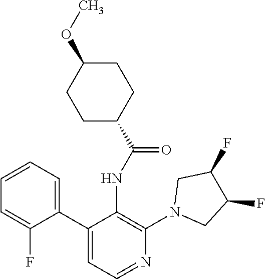 [SMILES: CO[C@H]1CC[C@H](C(=O)Nc2c(-c3ccccc3F)ccnc2N2C[C@@H](F)[C@@H](F)C2)CC1] | (1r,4R)-N-(2-((3S,4R)- 3,4-difluoropyrrolidin- 1-yl)-4-(2-fluorophen- yl)pyridin-3-yl)-4-meth- oxycyclohexane-1- carboxamide |
| 299 | 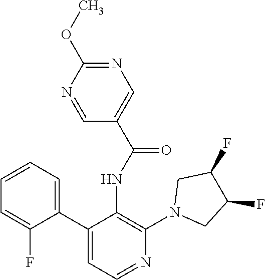 [SMILES: COc1ncc(C(=O)Nc2c(-c3ccccc3F)ccnc2N2C[C@@H](F)[C@@H](F)C2)cn1] | N-(2-((3S,4R)-3,4- difluoropyrrolidin-1-yl)- 4-(2-fluorophenyl)pyri- din-3-yl)-2-methoxypyr- imidine-5-carboxamide |
| 300 | 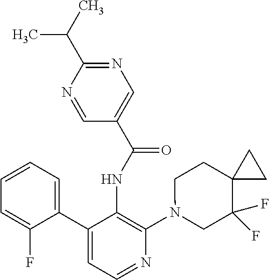 [SMILES: CC(C)c1ncc(C(=O)Nc2c(-c3ccccc3F)ccnc2N2CCC3(CC3)C(F)(F)C2)cn1] | N-(2-((3S,4R)-3,4- difluoropyrrolidin-1-yl)- 4-(2-fluorophenyl)pyri- din-3-yl)-2-isopropylpyr- imidine-5-carboxamide |
| 301 | 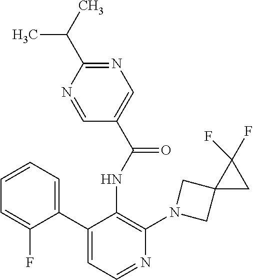 [SMILES: CC(C)c1ncc(C(=O)Nc2c(-c3ccccc3F)ccnc2N2CC3(C2)CC3(F)F)cn1] | N-(2-(1,1-difluoro-5- azaspiro[2.3]hexan-5- yl)-4-(2-fluorophenyl)- pyridin-3-yl)-2-isoprop- ylpyrimidine-5-carbox- amide |
| 302 | 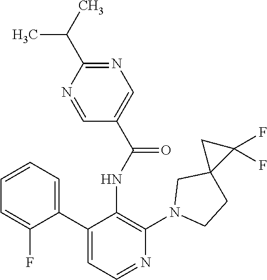 [SMILES: CC(C)c1ncc(C(=O)Nc2c(-c3ccccc3F)ccnc2N2CCC3(C2)CC3(F)F)cn1] | N-(2-(1,1-difluoro-5- azaspiro[2.4]heptan-5- yl)-4-(2-fluorophenyl)- pyridin-3-yl)-2-isoprop- ylpyrimidine-5-carbox- amide |
| 303 | 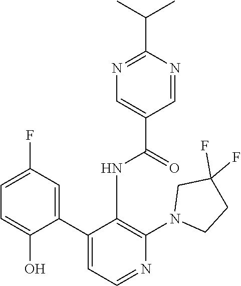 [SMILES: CC(C)c1ncc(C(=O)Nc2c(-c3cc(F)ccc3O)ccnc2N2CCC(F)(F)C2)cn1] | N-(2-(3,3-difluoropyr- rolidin-1-yl)-4-(5-fluoro- 2-hydroxyphenyl)pyri- din-3-yl)-2-isopropyl- pyrimidine-5-carbox- amide |
| 304 | 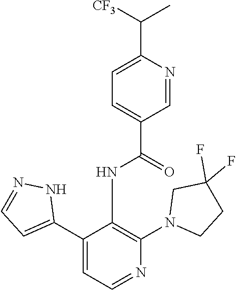 [SMILES: CC(c1ccc(C(=O)Nc2c(-c3ccn[nH]3)ccnc2N2CCC(F)(F)C2)cn1)C(F)(F)F] | N-(2-(3,3-difluoropyr- rolidin-1-yl)-4-(1H-pyr- azol-5-yl)pyridin-3-yl)- 6-(1,1,1-trifluoropropan- 2-yl)nicotinamide |
| 305 | 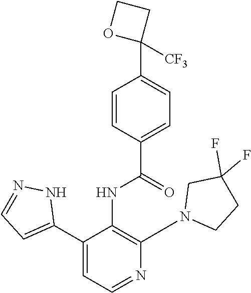 [SMILES: O=C(Nc1c(-c2ccn[nH]2)ccnc1N1CCC(F)(F)C1)c1ccc(C2(C(F)(F)F)CCO2)cc1] | N-(2-(3,3-difluoropyr- rolidin-1-yl)-4-(1H- indazol-5-yl)pyridin-3- yl)-2-isopropylpyrimi- dine-5-carboxamide P1 |
| 306 | 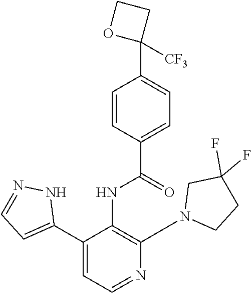 [SMILES: O=C(Nc1c(-c2ccn[nH]2)ccnc1N1CCC(F)(F)C1)c1ccc(C2(C(F)(F)F)CCO2)cc1] | N-(2-(3,3-difluoropyr- rolidin-1-yl)-4-(1H- indazol-5-yl)pyridin-3- yl)-2-isopropylpyrimi- dine-5-carboxamide P2 |
| 307 | 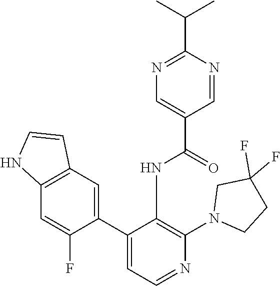 [SMILES: CC(C)c1ncc(C(=O)Nc2c(-c3cc4cc[nH]c4cc3F)ccnc2N2CCC(F)(F)C2)cn1] | N-(2-(3,3-difluoropyr- rolidin-1-yl)-4-(6-fluoro- 1H-indol-5-yl)pyridin- 3-yl)-2-isopropylpyrimi- dine-5-carboxamide |

Another embodiment of the disclosure is a pharmaceutical composition comprising one or more pharmaceutically acceptable excipient(s) and a therapeutically effective amount of a compound of formula (1) or (II), as described above in the Brief Summary, as a stereoisomer, enantiomer, or tautomer thereof or mixtures thereof; or a pharmaceutically acceptable salt, solvate, or prodrug thereof.

Another embodiment of the disclosure is a method of treating a disease or condition in a mammal modulated by a voltage-gated sodium channel, wherein the method comprises administering to a mammal in need thereof a therapeutically effective amount of a compound of formula (I) or (II), as described above in the Summary of the disclosure, as a stereoisomer, enantiomer, or tautomer thereof or mixtures thereof; or a pharmaceutically acceptable salt, solvate, or prodrug thereof.

Another embodiment of the disclosure is a method of using the compounds of formula (I) or (II) as standards or controls in in vitro or in vivo assays in determining the efficacy of test compounds in modulating voltage-dependent sodium channels.

Specific embodiments of the compounds of the disclosure are described in more detail below in the Compound Preparation section.

### Utility and Testing of the Compounds of the Disclosure

In an embodiment, the present disclosure is directed to compounds of formula (1) or (II), as individual stereoisomers, enantiomers, or tautomers thereof or mixtures thereof; or pharmaceutically acceptable salts, solvates, or prodrugs thereof, which are useful in treating seizure disorders, for example, epilepsy and/or epileptic seizure disorders, in a mammal, preferably a human.

In another embodiment, compounds of formula (1) or (II), as individual stereoisomers, enantiomers, or tautomers thereof or mixtures thereof; or pharmaceutically acceptable salts, solvates, or prodrugs thereof, disclosed herein are useful in treating epilepsy, seizure disorders, partial seizures (such as simple, complex, secondary generalized, and focal onset), generalized seizures (such as absence, myoclonic, atonic, tonic and tonic clonic), and disorders including photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures plus (GEFS+), Rett syndrome, multiple sclerosis, Schizophrenia, autism, ataxia, hypotonia and paroxysmal dyskinesia, Alzheimer's disease and Tauopathies, including but not limited to Alzheimer's disease, Pick's disease, progressive supranuclear palsy, corticobasal syndrome, frontotemporal dementias, Argyrophilic grain disease, frontotemporal lobar degeneration, globular glial tauopathies, MAPT mutation, primary age-related tauopathy, neurofibrillary tangle dementia, chronic traumatic encephalopathy (CTE), aging-related tau astrogliopathy, Richardson syndrome, Down Syndrome, parkinsonism, pure akinesia with gait freezing, motor neuron symptoms or cerebellar ataxia, posttraumatic stress disorders (PTSD) or any combination of the these.

The present disclosure readily affords many different means for identification of sodium channel modulating agents that are useful as therapeutic agents. Identification of modulators of sodium channels can be assessed using a variety of in vitro and in vivo assays, e.g., measuring current, measuring membrane potential, measuring ion flux, (e.g., sodium), measuring sodium concentration, measuring second messengers and transcription levels, measuring neurotransmitter levels and using voltage-sensitive dyes, radioactive tracers, multi-electrode-arrays and patch-clamp electrophysiology.

One such protocol involves the screening of chemical agents for ability to modulate the activity of a sodium channel thereby identifying it as a modulating agent.

A typical assay described in (Crestey, F. et al., ACS Chem Neurosci (2015), Vol. 6, pp. 1302-1308), AA43279 (Frederiksen, K. et a/., Eur J Neurosci (2017), Vol. 46, pp. 1887-1896) and Lu AE98134 (von Schoubyea, N. L. et al., Neurosci Lett (2018), Vol. 662, pp. 29-35) employs the use of automated planar patch clamp techniques to study the effects of the chemical agent on the gating of sodium channels.

The sodium channel isoforms of interest are stably expressed in Human Embryonic Kidney Cells and the curretns that flow through those channels in response to a depolarizing voltage clamp step from −120 mV to 0 mV are measured in the presence of increasing concentrations of the chemical agents. The area under the sodium current trace which correlates to the magnitude of sodium flux through the cell mebrane is used to quantify the effects on gating of the channels. Other parameters that are measured in the assay include the peak current, time constant of open state inactivation and the voltage dependence of steady state inactivation properties. The concentration responses are used to determine potency of each chemical agents effects on modulating the sodium channel isoform gatingSuch techniques are known to those skilled in the art, and may be developed, using current technologies, into low or medium throughput assays for evaluating compounds for their ability to modulate sodium channel behaviour.

The results of these assays provide the basis for analysis of the structure-activity relationship (SAR) between compounds of the disclosure and the sodium channel. Certain substituents on the core structure of a compound of the disclosure tend to provide more potent inhibitory or potentiating compounds. SAR analysis is one of the tools those skilled in the art may now employ to identify preferred embodiments of the compounds of the disclosure for use as therapeutic agents.

In an alternative use of the disclosure, the compounds of the disclosure can be used in in vitro or in vivo studies as exemplary agents for comparative purposes to find other compounds also useful in treatment of, or protection from, the various diseases disclosed herein.

In another embodiment, the compounds of formula (1) or (II), as individual stereoisomers, enantiomers, or tautomers thereof or mixtures thereof; or pharmaceutically acceptable salts, solvates, or prodrugs thereof, as set forth above in the Brief Summary, as stereoisomers, enantiomers, tautomers thereof or mixtures thereof, or pharmaceutically acceptable salts, solvates, or prodrugs thereof, and/or the pharmaceutical compositions described herein which comprise a pharmaceutically acceptable excipient and one or more compounds of the disclosure, as set forth above in the Brief Summary, as a stereoisomer, enantiomer, or tautomer thereof or mixtures thereof, or a pharmaceutically acceptable salt, solvate, or prodrug thereof, can be used in the preparation of a medicament for the treatment of a sodium channel-mediated disease or condition in a mammal.

### Pharmaceutical Compositions and Administration

This disclosure is also directed to pharmaceutical compositions containing the compounds of formula (I) or (II), as described above in the Brief Summary, as stereoisomers, enantiomers, or tautomers thereof or mixtures thereof; or pharmaceutically acceptable salts, solvates, or prodrugs thereof. In one embodiment, the present disclosure relates to a pharmaceutical composition comprising compounds of formula (1) or (II), as described above in the Brief Summary, as stereoisomers, enantiomers, or tautomers thereof or mixtures thereof; or pharmaceutically acceptable salts, solvates, or prodrugs thereof, in a pharmaceutically acceptable carrier, excipient or diluent and in an amount effective to modulate, preferably inhibit, voltage-gated sodium channels to treat certain diseases or conditions, such as epilepsy, when administered to an animal, preferably a mammal, most preferably a human patient.

Administration of the compounds of formula (I) or (II), as described above in the Brief Summary, as stereoisomers, enantiomers, or tautomers thereof or mixtures thereof; or pharmaceutically acceptable salts, solvates, or prodrugs thereof, in pure form or in an appropriate pharmaceutical composition, can be carried out via any of the accepted modes of administration of agents for serving similar utilities. The pharmaceutical compositions of the disclosure can be prepared by combining a compound of the disclosure with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term “parenteral” as used herein includes subcutaneous injections, intravenous, intramuscular, intrathecal, intrasternal injection or infusion techniques. Pharmaceutical compositions of the disclosure are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings of this disclosure.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained. Typically, this amount is at least 0.01% of a compound of the disclosure in the composition. When intended for oral administration, this amount may be varied to be between 0.1 and about 70% of the weight of the composition. Preferred oral pharmaceutical compositions contain between about 4% and about 50% of the compound of the disclosure. Preferred pharmaceutical compositions and preparations according to the present disclosure are prepared so that a parenteral dosage unit contains between 0.01 to 10% by weight of the compound prior to dilution of the disclosure.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the disclosure from about 0.1 to about 10% w/v (weight per unit volume).

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, a protein or a liposome.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining a compound of the disclosure with sterile, distilled water so as to form a solution. A surfactant may be added to facilitate the formation of a homogeneous solution or suspension. Surfactants are compounds that non-covalently interact with the compound of the disclosure so as to facilitate dissolution or homogeneous suspension of the compound in the aqueous delivery system.

The compounds of the disclosure, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkowet al., eds., The Merck Manual, 16^{th} edition, Merck and Co., Rahway, N.J., 1992; Goodmanetna., eds.,Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, MD. (1987), Ebadi, Pharmacology, Little, Brown and Co., Boston, (1985); Osolci al., eds., Remington's Pharmaceutical Sciences, 18^{th} edition, Mack Publishing Co., Easton, PA (1990); Katzung, Basic and Clinical Pharmacology, Appleton and Lange, Norwalk, CT (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the disclosure can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the disclosure to target area, e.g., skin surfaces, mucous membranes, and the like, which are adjacent to peripheral neurons which are to be treated. This amount will generally range from about 0.0001 mg to about 1 g of a compound of the disclosure per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present disclosure as desired.

The compositions of the disclosure can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al., Regional Anesthesia 22 (6): 543-551 (1997), all of which are incorporated herein by reference.

The compositions of the disclosure can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The disclosure also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Published Patent Application No. WO 98/50016.

Current methods for ocular delivery include topical administration (eye drops), subconjunctival injections, periocular injections, intravitreal injections, surgical implants and iontophoresis (uses a small electrical current to transport ionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (e.g., oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the disclosure may be usefully combined with one or more other compounds of the disclosure or one or more other therapeutic agent or as any combination thereof, in the treatment of sodium channel-mediated diseases and conditions. For example, a compound of this disclosure may be administered simultaneously, sequentially, or separately in combination with other therapeutic agents, including, but not limited to:

Acetazolamide (Diamox), Brivaracetam (Briviact), Cannabidiol (Epidiolex), Carbamazepine (Tegretol), Cenobamate (Xcopri), Clobazam (Frisium), Clonazepam (Klonopin), Eslicarbazepine acetate (Aptiom, Zebinix), Ethosuximide (Zarontin), Felbamate (Felbatol), Fenfluramine (Fintepla), Gabapentin (Neurontin), Lacosamide (Vimpat), Lamotrigine (Lamictal), Levetiracetam (Keppra), Oxcarbazepine (Trileptal), Perampanel (Fycompa), Phenobarbital (Luminal), Phenytoin (Dilantin), Pregabalin (Lyrica), Primidone, Retigabine (Ezogabine), Rufinamide (Banzel), Stiripentol (Diacomit), Sulthiame, Tiagabine (Gabitril), Topiramate (Topamax), Valproate (Depakote), Vigabatrin (Sabril), Zonisamide (Zonegran).

As used herein “combination” refers to any mixture or permutation of one or more compounds of the disclosure and one or more other compounds of the disclosure or one or more additional therapeutic agent. Unless the context makes clear otherwise, “combination” may include simultaneous or sequentially delivery of a compound of the disclosure with one or more therapeutic agents. Unless the context makes clear otherwise, “combination” may include dosage forms of a compound of the disclosure with another therapeutic agent. Unless the context makes clear otherwise, “combination” may include routes of administration of a compound of the disclosure with another therapeutic agent. Unless the context makes clear otherwise, “combination” may include formulations of a compound of the disclosure with another therapeutic agent. Dosage forms, routes of administration and pharmaceutical compositions include, but are not limited to, those described herein.

### Kits-of-Parts

The present disclosure also provides kits that contain a pharmaceutical composition which includes one or more compounds of the disclosure. The kit also includes instructions for the use of the pharmaceutical composition for modulating the activity of sodium channels, for the treatment of a seizure disorder, such as epilepsy, as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Compound Preparation

The following Reaction Schemes illustrate methods to make compounds of the disclosure, i.e., compounds of formula (I) or (II), as described above in the Brief Summary, as stereoisomers, enantiomers, or tautomers thereof or mixtures thereof; or pharmaceutically acceptable salts, solvates, or prodrugs thereof.

It is also understood that one skilled in the art would be able to make the compounds of the disclosure by similar methods or by methods known to one skilled in the art. It is also understood that one skilled in the art would be able to make in a similar manner as described below other compounds of the disclosure not specifically illustrated below by using the appropriate starting components and modifying the parameters of the synthesis as needed. In general, starting components may be obtained from sources such as Sigma Aldrich, Alfa Aesar, Combi-Blocks, Oakwood Chemicals, Matrix Scientific, and TCI, etc. or synthesized according to sources known to those skilled in the art (see, e.g., M. B. Smith and J. March, Advanced Organic Chemistry: Reactions, Mechanisms, and Structure, 6th edition (Wiley, 2007)) or prepared as described herein.

It is also understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, include t-butoxycarbonyl, benzyloxycarbonyl, β-methoxybenzyl, trityl and the like.

Protecting groups may be added or removed in accordance with standard techniques, which are known to one skilled in the art and as described herein.

The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. All prodrugs of compounds of formula (I) or (II) are included within the scope of the disclosure.

The compounds of formula (I) or (II) may contain at least one asymmetric carbon atom and thus can exist as racemates, enantiomers, and/or diastereoisomers. Specific enantiomers, or diastereoisomers may be prepared by utilizing the appropriate chiral starting material or through the use of suitable asymmetric synthetic methods. Alternatively, diastereoisomeric mixtures or racemic mixtures of compounds of formula (I) or (II) may be resolved into their respective enantiomers or diastereoisomers.

Methods for resolution of diastereoisomeric mixtures or racemic mixtures of the compounds of formula (I) or (II), as described herein, or intermediates prepared herein, are well known in the art (e.g., E. L. Eliel and S. H. Wilen, in Stereochemistry of Organic Compounds; John Wiley & Sons: New York, 1994; Chapter 7, and references cited therein). Suitable processes such as crystallization (e.g., preferential crystallization, preferential crystallization in the presence of additives), asymmetric transformation of racemates, chemical separation (e.g., formation and separation of diastereomers such as diastereomeric salt mixtures or the use of other resolving agents; separation via complexes and inclusion compounds), kinetic resolution (e.g., with titanium tartrate catalyst), enzymatic resolution (e.g., lipase mediated) and chromatographic separation (e.g., HPLC with chiral stationary phase and/or with simulated moving bed technology, or supercritical fluid chromatography and related techniques) are some of the examples that may be applied (see e.g., T.J. Ward, Analytical Chemistry, 2002, 2863-2872).

In general, compounds of formula (I) or (II), as described above in the Brief Summary, can be synthesized following the general procedures described below in Reaction Schemes 1-2 wherein X, R¹, R², R³, R⁴, and R⁷ are as defined herein and Z¹ is a suitable coupling partner to Z³, for example, halo such as iodo or chloro, Z² is a suitable coupling partner to R^{2a}—NH—R^{2b}, for example, halo such as chloro, Z³ is a suitable coupling partner to Z¹, for example, a boronic acid or ester. Additionally, the reagent R^{2a}—NH—R^{2b} is selected based on the desired R². Similarly, R^{3′}—NH₂ is selected based on the desired R³. In some embodiments, R^{3′}—NH₂ is substituted with R^{3′}—NH to afford the desired R³ (e.g., 4-methoxypiperidine when R³ is 4-methoxypiperidinyl or 7-methoxy-2-azaspiro[3.5]nonane when R³ is 7-methoxy-2-azaspiro[3.5]nonanyl). In some embodiments, X¹ is, at each occurrence, a substituent that facilitates the desired reaction (e.g., —OCl₃— that is, in some embodiments, X¹—C(═O)—X¹ is triphosgene).

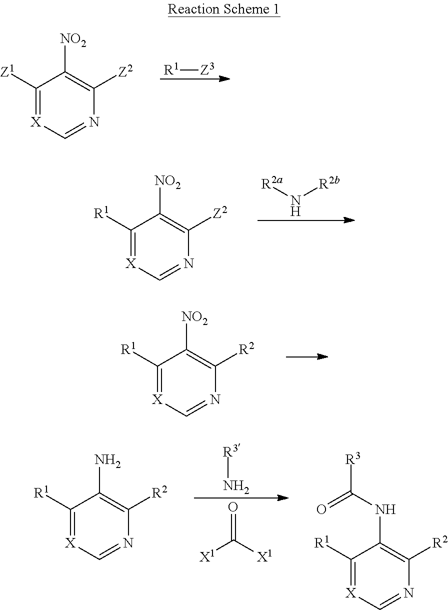

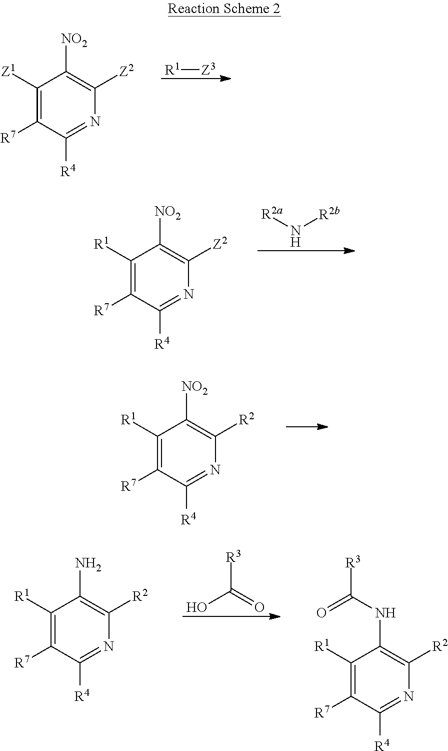

All of the compounds described below as being prepared which may exist in free base or acid form may be converted to their pharmaceutically acceptable salts by treatment with the appropriate inorganic or organic base or acid. Salts of the compounds prepared below may be converted to their free base or acid form by standard techniques. Furthermore, all compounds of the disclosure which contain an acid or an ester group can be converted to the corresponding ester or acid, respectively, by methods known to one skilled in the art or by methods described herein.

The present disclosure also relates to novel intermediate compounds as defined above, all salts, solvates, and complexes thereof and all solvates and complexes of salts thereof as defined hereinbefore for compounds of formula (I) or (II). The disclosure includes all polymorphs of the aforementioned species and crystal habits thereof.

Embodiments disclosed herein are also meant to encompass all compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively.

Isotopically-labeled compounds can generally be prepared by conventional techniques known to those skilled in the art or by processes analogous to those described below and in the following Examples using an appropriate isotopically-labeled reagent in place of the non-labeled reagent previously employed.

The following Examples, which are directed to the synthesis of the compounds of the disclosure; and the following Biological Examples are provided as a guide to assist in the practice of the disclosure, and are not intended as a limitation on the scope of the disclosure.

In the Preparations and Examples below, unless otherwise indicated all temperatures are set forth in degrees Celsius. Commercially available reagents were purchased from suppliers such as Sigma Aldrich, Alfa Aesar, Combi-Blocks, Oakwood Chemicals, Matrix Scientific, and TCI, etc. and were used without further purification unless otherwise indicated. The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried. Yields were not optimized. Melting points were determined on a Buchi hot-stage apparatus and are uncorrected. ¹H NMR, ¹⁹F and ¹³C NMR data were obtained in deuterated CDCl₃, DMSO-d₆, CD₃OD, CD₃CN, or acetone-d₆ solvent solutions with chemical shifts (δ) reported in parts-per-million (ppm) relative to trimethylsilane (TMS) or the residual non-deuterated solvent peaks as the reference standard. Data are reported as follows, if applicable: chemical shift, multiplicity, coupling constant in Hz, and number of protons, fluorine or carbon atoms. When peak multiplicities are reported, the following abbreviates are used: s (singlet), d (doublet), t (triplet), q (quartet), m (multiplet, br (broadened), dd (doublet of doublets), dt (doublet of triplets). Coupling constants, when given, are reported in Hz (Hertz).

### Example 1

### Synthesis of 1-(4-(2-fluorophenyl)-2-(pyrrolidin-1-yl)pyridin-3-yl)-3-(4-isopropylphenyl)urea

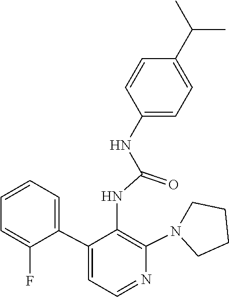 [SMILES: CC(C)c1ccc(NC(=O)Nc2c(-c3ccccc3F)ccnc2N2CCCC2)cc1]

### Step 1. Preparation of 4-(2-fluorophenyl)-3-nitro-2-(pyrrolidin-1-yl)pyridine

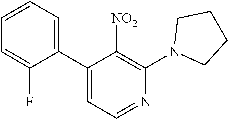 [SMILES: O=[N+]([O-])c1c(-c2ccccc2F)ccnc1N1CCCC1]

To a solution of 2-chloro-4-(2-fluorophenyl)-3-nitropyridine (1.66 g, 6.59 mmol) in anhydrous acetonitrile (15 mL) was added triethylamine (2.75 mL, 19.8 mmol), and pyrrolidine (0.55 mL, 6.60 mmol) at 0° C. The reaction mixture was stirred at ambient temperature for 2 h 40 min. Then the reaction mixture was filtered, and the filtrate was diluted with DCM (50 mL) and washed with 1 M aqueous hydrogen chloride solution (50 mL), water (50 mL) and brine (50 mL). The organic phase was dried over anhydrous sodium sulfate, and filtered. The filtrate was concentrated in vacuo to give the title compound as a yellow solid (2.03 g, >99% yield): ¹H-NMR (300 MHz; CDCl₃) δ 8.28 (d, J=4.9 Hz, 1H), 7.44-7.36 (m, 1H), 7.28-7.11 (m, 3H), 6.54 (d, J=4.9 Hz, 1H), 3.48-3.44 (m, 4H), 1.99-1.95 (m, 4H); MS (ES+) m/z 288.4 (M+1).

### Step 2. Preparation of 4-(2-fluorophenyl)-2-(pyrrolidin-1-yl)pyridin-3-amine

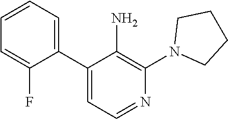 [SMILES: Nc1c(-c2ccccc2F)ccnc1N1CCCC1]

To a solution of 4-(2-fluorophenyl)-3-nitro-2-(pyrrolidin-1-yl)pyridine (2.038 g, 7.09 mmol) in glacial acetic acid (35 mL) was added iron power (2.38 g, 42.6 mmol). The reaction mixture was heated to 60° C. for 2 h. Then the reaction mixture was poured onto ice and was neutralized with saturated sodium bicarbonate and sodium carbonate solution till the pH reached 6.5. The mixture was extracted with ethyl acetate (3×100 mL). The combined organic phases were washed with saturated aqueous sodium bicarbonate (150 mL) and brine (100 mL), dried over anhydrous sodium sulfate, and filtered. The filtrate was concentrated in vacuo to give the title compound in quantitative yield: ¹H-NMR (300 MHz; CDCl₃) δ 7.84 (d, J=5.4 Hz, 1H), 7.49-7.37 (m, 2H), 7.32-7.20 (m, 2H), 6.77 (d, J=5.3 Hz, 1H), 3.86 (s, 2H), 3.64-3.61 (m, 4H), 2.05-1.97 (m, 4H); MS (ES+) m/z 258.4 (M+1).

### Step 3. Preparation of 1-(4-(2-fluorophenyl)-2-(pyrrolidin-1-yl)pyridin-3-yl)-3-(4-isopropylphenyl)urea

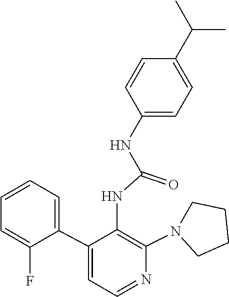 [SMILES: CC(C)c1ccc(NC(=O)Nc2c(-c3ccccc3F)ccnc2N2CCCC2)cc1]

To a solution of 4-(2-fluorophenyl)-3-nitro-2-(pyrrolidin-1-yl)pyridine (0.065 g, 0.253 mmol) in anhydrous 1,4-dioxane (1.0 mL) was added 1-isocyanato-4-isopropylbenzene (0.048 mL, 0.30 mmol). The reaction mixture was stirred at ambient temperature for 16 h, before concentrated in vacuo to give a residue. This residue was purified by column chromatography, eluting with a gradient of 5% to 100% of ethyl acetate in heptane, to provide the title compound as a colorless solid (0.065 g, 62% yield): ¹H-NMR (300 MHz; DMSO-d₆) δ 8.41 (br s, 1H), 8.05 (d, J=4.9 Hz, 1H), 7.43-7.31 (m, 3H), 7.28-7.17 (m, 2H), 7.14-7.10 (m, 2H), 7.04-7.01 (m, 2H), 6.56 (dd, J=4.9, 0.7 Hz, 1H), 3.56-3.52 (m, 4H), 2.81-2.71 (m, 1H), 1.86-1.81 (m, 4H), 1.13 (d, J=6.9 Hz, 6H); MS (ES+) m/z 419.4 (M+1).

### Example 2

### Synthesis of 1-butyl-3-(4-(2-fluorophenyl)-2-(pyrrolidin-1-yl)pyridin-3-yl)urea

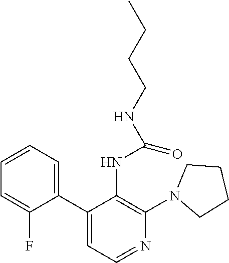 [SMILES: CCCCNC(=O)Nc1c(-c2ccccc2F)ccnc1N1CCCC1]

To a solution of 4-(2-fluorophenyl)-3-nitro-2-(pyrrolidin-1-yl)pyridine (0.095 g, 0.37 mmol) in anhydrous 1,4-dioxane (1.0 mL) was added 1-isocyanatobutane (0.050 mL, 0.44 mmol). The reaction mixture was stirred at ambient temperature for 24 h, before concentrated in vacuo to give a residue. This residue was was purified by column chromatography, eluting with a gradient of 10% to 100% of ethyl acetate in heptane, to provide the title compound as colorless solid (0.064 g, 48% yield): ¹H-NMR (300 MHz; DMSO-d₆): δ 8.00 (d, J=4.9 Hz, 1H), 7.42-7.35 (m, 1H), 7.32-7.25 (m, 1H), 7.23-7.16 (m, 2H), 6.51 (dd, J=4.9, 0.9 Hz, 1H), 5.82-5.76 (m, 1H), 3.51 (t, J=6.4 Hz, 4H), 2.86-2.80 (m, 2H), 1.85-1.81 (m, 4H), 1.18-1.01 (m, 4H), 0.78 (q, J=4.7 Hz, 3H); MS (ES+) m/z 357.4 (M+1).

### Example 3

### (S)-2-chloro-N-(4-(2,5-difluorophenyl)-2-(3-fluoropyrrolidin-1-yl)pyridin-3-yl)pyrimidine-5-carboxamide formic acid salt

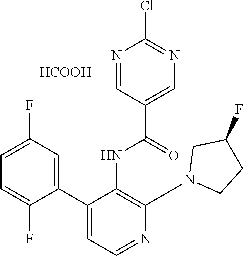 [SMILES: O=C(Nc1c(-c2cc(F)ccc2F)ccnc1N1CC[C@H](F)C1)c1cnc(Cl)nc1 ; "HCOOH"]

### Step 1. Preparation of 2-chloro-4-(2,5-difluorophenyl)-3-nitropyridine

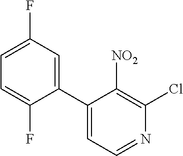 [SMILES: O=[N+]([O-])c1c(-c2cc(F)ccc2F)ccnc1Cl]

To a mixture of 2,4-dichloro-3-nitropyridine (8.00 g, 41.5 mmol) in dioxane (162 mL) and water (54 mL) was added (2,5-difluorophenyl)boronic acid (6.55 g, 41.5 mmol), dichloro 1,1′-bis(diphenylphosphino)ferrocene palladium (II) dichloromethane (1.52 g, 1.86 mmol), and potassium carbonate (17.19 g, 124.4 mmol). The reaction mixture was stirred at 60° C. for 45 minutes. After cooling to ambient temperature, the mixture was diluted with water (500 mL) and extracted with ethyl acetate (3×500 mL). The combined organic solution was washed with brine (1000 mL), dried over anhydrous sodium sulfate, filtered and concentrated in vacuo. Purification of the residue by reverse-phase column chromatography, using acetonitrile in water containing 0.1% formic acid as eluent, afforded the title compound as a colorless solid (7.00 g, 62% yield): MS (ES+) m/z 271.3 (M+1).

### Step 2. Preparation of (S)-4-(2,5-difluorophenyl)-2-(3-fluoropyrrolidin-1-yl)-3-nitropyridine

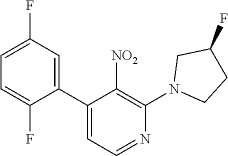 [SMILES: O=[N+]([O-])c1c(-c2cc(F)ccc2F)ccnc1N1CC[C@H](F)C1]

To a mixture of 2-chloro-4-(2,5-difluorophenyl)-3-nitropyridine (3.60 g, 12.3 mmol) in acetonitrile (35 mL) was added potassium carbonate (5.52 g, 39.9 mmol), and (S)-3-fluoropyrrolidine hydrochloride (1.84 g, 14.6 mmol). The reaction mixture was stirred at ambient temperature for 16 h. The mixture was filtered and concentrated in vacuo. Purification of the residue by column chromatography, eluting with a gradient of 5 to 6% ethyl acetate in petroleum ether, afforded the title compound as a yellow oil (3.00 g, 75% yield): MS (ES+) m/z 324.3 (M+1).

### Step 3. Preparation of (S)-4-(2,5-difluorophenyl)-2-(3-fluoropyrrolidin-1-yl)pyridin-3-amine

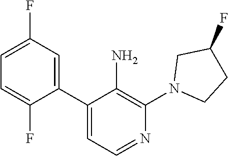 [SMILES: Nc1c(-c2cc(F)ccc2F)ccnc1N1CC[C@H](F)C1]

To a mixture of (S)-4-(2,5-difluorophenyl)-2-(3-fluoropyrrolidin-1-yl)-3-nitropyridine (3.00 g, 9.28 mmol) in methanol (20 mL) degassed with nitrogen was added 10% weight palladium on carbon (0.350 g). The reaction mixture was degassed under vacuum and purged with hydrogen several times, and was stirred at ambient temperature for 1 h under an atmosphere of hydrogen. The mixture was filtered and concentrated in vacuo. Purification of the residue by column chromatography, eluting with a gradient of 2 to 10% ethyl acetate in petroleum ether, afforded the title compound as a colorless solid (1.70 g, 62% yield): MS (ES+) m/z 294.3 (M+1).

### Step 4. Preparation (S)-2-chloro-N-(4-(2,5-difluorophenyl)-2-(3-fluoropyrrolidin-1-yl)pyridin-3-yl)pyrimidine-5-carboxamide formic acid salt

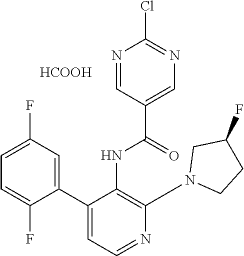 [SMILES: O=C(Nc1c(-c2cc(F)ccc2F)ccnc1N1CC[C@H](F)C1)c1cnc(Cl)nc1 ; "HCOOH"]

To a mixture of (S)-4-(2,5-difluorophenyl)-2-(3-fluoropyrrolidin-1-yl)pyridin-3-amine (0.100 g, 0.341 mmol) in tetrahydrofuran (4.0 mL) was added 2-chloropyrimidine-5-carboxylic acid (0.350 g, 0.33 mmol), 50% weight 2,4,6-tripropyl-1,3,5,2,4,6-trioxatriphosphinane 2,4,6-trioxide in ethyl acetate (0.325 g, 0.511 mmol) and N,N-diisopropylamine (0.088 g, 0.682 mmol). The reaction mixture was stirred at 70° C. for 12 h. After cooling to ambient temperature, the mixture was diluted with water (20 mL) and extracted with ethyl acetate (3×20 mL). The combined organic solution was washed with brine (3×30 mL), dried over anhydrous sodium sulfate, filtered and concentrated in vacuo. Purification of the residue by reverse-phase column chromatography, using acetonitrile in water containing 0.1% formic acid as eluent, afforded a residue that was further purified by preparatory TLC, using 50% ethyl acetate in petroleum ether as eluent, and subsequently purified by preparative reverse-phase HPLC, using 27 to 57% acetonitrile in water containing 0.2% formic acid as eluent, afforded the title compound as a yellow solid (0.017 g, 10% yield): ¹H NMR (400 MHz, CD₃OD) δ8.89-8.86 (m, 2H), 8.46 (br s, 0.3H), 8.19 (d, J=5.0 Hz, 1H), 7.21-7.07 (m, 3H), 6.74 (d, J=5.0 Hz, 1H), 5.36-5.21 (m, 1H), 3.90-3.67 (m, 4H), 2.26-2.01 (m, 2H); MS (ES+) m/z 434.2 (M+1).

### Example 4-15

In a similar manner as described in EXAMPLE 3, utilizing the appropriately substituted starting materials and intermediates, the following compounds were prepared:

| | | | Amount (g) | |
|---|---|---|---|---|
| | | | Yield % | |
| Example | | | MS (ES+) | |
| No. | Structure | Name | m/z | ¹H NMR |
| 4 | 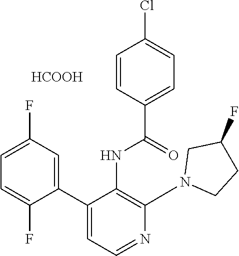 [SMILES: O=C(Nc1c(-c2cc(F)ccc2F)ccnc1N1CC[C@H](F)C1)c1ccc(Cl)cc1 ; "HCOOH"] | (S)-4-chloro-N- (4-(2,5- difluorophenyl)- 2-(3- fluoropyrrolidin- 1-yl)pyridin-3- yl)benzamide formic acid salt | 0.016 g 5% 431.1 (M + 1) | (400 MHz, CD₃OD) δ 8.50 (br s, 0.2 H), 8.16 (d, J = 5.0 Hz, 1H), 7.68-7.65 (m, 2H), 7.46-7.43 (m, 2H), 7.16-7.04 (m, 3H), 6.72 (d, J = 5.0 Hz, 1H), 5.34- 5.19 (m, 1H), 3.89- 3.68 (m, 4H), 2.27- 1.96 (m, 2H) |
| 5 | 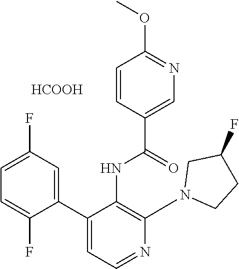 [SMILES: COc1ccc(C(=O)Nc2c(-c3cc(F)ccc3F)ccnc2N2CC[C@H](F)C2)cn1 ; "HCOOH"] | (S)-N-(4-(2,5- difluorophenyl)- 2-(3- fluoropyrrolidin- 1-yl)pyridin-3- yl)-6- methoxynicotinamide formic acid salt | 0.018 g 11% 429.2 (M + 1) | (400 MHz, CD₃OD) δ 8.49 (d, J = 2.5 Hz, 1H), 8.45 (br s, 0.2H), 8.15 (d, J = 5.0 Hz, 1H), 7.94 (dd, J = 8.7, 2.5 Hz, 1H), 7.17-7.04 (m, 3H), 6.81 (d, J = 8.7 Hz, 1H), 6.72 (d, J = 5.0 Hz, 1H), 5.34-5.19 (m, 1H), 3.95 (s, 3H), 3.89- 3.69 (m, 5H), 2.28- 1.97 (m, 2H) |
| 6 | 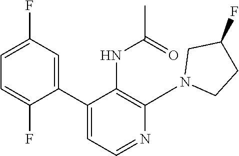 [SMILES: CC(=O)Nc1c(-c2cc(F)ccc2F)ccnc1N1CC[C@H](F)C1] | (S)-N-(4-(2,5- difluorophenyl)- 2-(3- fluoropyrrolidin- 1-yl)pyridin-3- yl) acetamide | 0.030 g 26% 336.3 (M + 1) | (400 MHz, CD₃OD) δ 8.10 (d, J = 5.0 Hz, 1H), 7.23-7.13 (m, 2H), 7.06-7.02 (m, 1H), 6.66 (d, J = 5.0 Hz, 1H), 5.39-5.24 (m, 1H), 3.91-3.65 (m, 4H), 2.32-2.12 (m, 2H), 1.84 (s, 3H) |
| 7 | 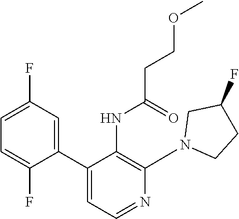 [SMILES: COCCC(=O)Nc1c(-c2cc(F)ccc2F)ccnc1N1CC[C@H](F)C1] | (S)-N-(4-(2,5- difluorophenyl)- 2-(3- fluoropyrrolidin- 1-yl)pyridin-3- yl)-3- methoxypropanamide | 0.010 g 14% 380.3 (M + 1) | (400 MHz, CD₃OD) δ 8.02 (d, J = 6.6 Hz, 1H), 7.31 (dd, J = 6.2, 6.2 Hz, 2H), 7.14-7.10 (m, 1H), 7.04 (d, J = 6.5 Hz, 1H), 5.53- 5.39 (m, 1H), 3.99 (m, J = 42.1, 16.7 Hz, 4H), 3.55-3.45 (m, 2H), 3.24 (s, 3H), 2.55-2.16 (m, 4H) |
| 8 | 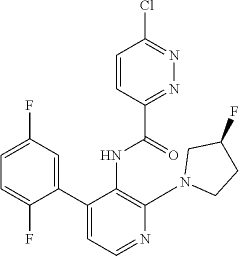 [SMILES: O=C(Nc1c(-c2cc(F)ccc2F)ccnc1N1CC[C@H](F)C1)c1ccc(Cl)nn1] | (S)-6-chloro-N- (4-(2,5- difluorophenyl)- 2-(3- fluoropyrrolidin- 1-yl)pyridin-3- yl)pyridazine-3- carboxamide | 0.050 g 67% 434.1 (M + 1) | (400 MHz, CD₃OD) δ 8.17 (d, J = 5.1 Hz, 1H), 8.11 (d, J = 8.9 Hz, 1H), 7.92 (d, J = 8.9 Hz, 1H), 7.16-7.00 (m, 3H), 6.74 (d, J = 5.1 Hz, 1H), 5.32-5.17 (m, 1H), 3.91-3.73 (m, 4H), 2.26-1.94 (m, 2H) |
| 9 | 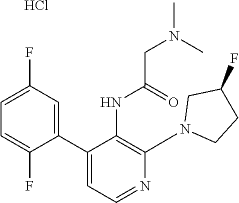 [SMILES: CN(C)CC(=O)Nc1c(-c2cc(F)ccc2F)ccnc1N1CC[C@H](F)C1 ; Cl] | (S)-N-(4-(2,5- difluorophenyl)- 2-(3- fluoropyrrolidin- 1-yl)pyridin-3- yl)-2- (dimethylamino) acetamide hydrochloric acid salt | 0.012 g 9% 379.3 (M + 1) | (400 MHz, CD₃OD) δ 8.10 (d, J = 6.2 Hz, 1H), 7.37-7.28 (m, 2H), 7.24-7.19 (m, 1H), 7.02 (d, J = 6.2 Hz, 1H), 5.54-5.40 (m, 1H), 4.17-3.88 (m, 6H), 2.74 (s, 6H), 2.53- 2.40 (m, 1H), 2.37- 2.17 (m, 1H) |
| 10 | 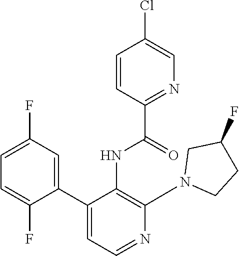 [SMILES: O=C(Nc1c(-c2cc(F)ccc2F)ccnc1N1CC[C@H](F)C1)c1ccc(Cl)cn1] | (S)-5-chloro-N- (4-(2,5- difluorophenyl)- 2-(3- fluoropyrrolidin- 1-yl)pyridin-3- yl)picolinamide | 0.195 g 65% 433.1 (M + 1) | (400 MHz, CD₃OD) δ 8.63 (dd, J = 2.2, 0.9 Hz, 1H), 8.16 (d, J = 5.1 Hz, 1H), 7.98-7.92 (m, 2H), 7.13 (ddd, J = 8.8, 5.6, 3.1 Hz, 1H), 7.09-6.99 (m, 2H), 6.73 (dd, J = 5.1, 0.5 Hz, 1H), 5.31- 5.15 (m, 1H), 3.88- 3.69 (m, 4H), 2.24- 1.94 (m, 2H) |
| 11 | 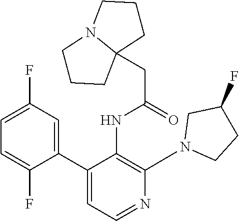 [SMILES: O=C(CC12CCCN1CCC2)Nc1c(-c2cc(F)ccc2F)ccnc1N1CC[C@H](F)C1] | (S)-N-(4-(2,5- difluorophenyl)- 2-(3- fluoropyrrolidin- 1-yl)pyridin-3- yl)-2- (tetrahydro-1H- pyrrolizin- 7a(5H)- yl)acetamide | 0.083 g 5% 445.3 (M + 1) | — |
| 12 | 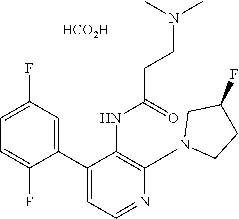 [SMILES: CN(C)CCC(=O)Nc1c(-c2cc(F)ccc2F)ccnc1N1CC[C@H](F)C1 ; "HCO2H"] | (S)-N-(4-(2,5- difluorophenyl)- 2-(3- fluoropyrrolidin- 1-yl)pyridin-3- yl)-3- (dimethylamino) propanamide formate salt | 0.011 g 7% 393.3 (M + 1) | — |
| 13 | 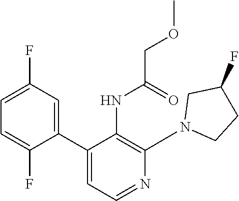 [SMILES: COCC(=O)Nc1c(-c2cc(F)ccc2F)ccnc1N1CC[C@H](F)C1] | (S)-N-(4-(2,5- difluorophenyl)- 2-(3- fluoropyrrolidin- 1-yl)pyridin-3- yl)-2- methoxyacetamide | 0.0182 g 29% 366.3 (M + 1) | — |
| 14 | 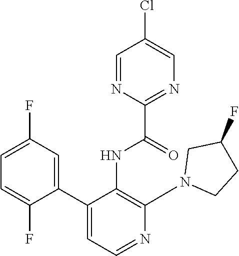 [SMILES: O=C(Nc1c(-c2cc(F)ccc2F)ccnc1N1CC[C@H](F)C1)c1ncc(Cl)cn1] | (S)-5-chloro-N- (4-(2,5- difluorophenyl)- 2-(3- fluoropyrrolidin- 1-yl)pyridin-3- yl)pyrimidine-2- carboxamide | 0.0175 g 11% 434.2 (M + 1) | — |
| 15 | 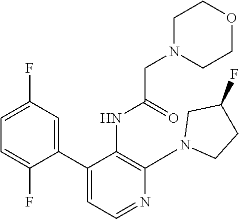 [SMILES: O=C(CN1CCOCC1)Nc1c(-c2cc(F)ccc2F)ccnc1N1CC[C@H](F)C1] | (S)-N-(4-(2,5- difluorophenyl)- 2-(3- fluoropyrrolidin- 1-yl)pyridin-3- yl)-2- morpholino- acetamide | 0.0325 g 21% 421.2 (M + 1) | — |

### Example 16

### Synthesis of (S)-N-(4-(2,5-difluorophenyl)-2-(3-fluoropyrrolidin-1-yl)pyridin-3-yl)-1-methylpiperidine-4-carboxamide

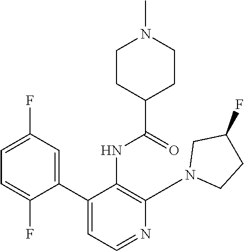 [SMILES: CN1CCC(C(=O)Nc2c(-c3cc(F)ccc3F)ccnc2N2CC[C@H](F)C2)CC1]

### Step 1. Preparation of (S)-tert-butyl 4-((4-(2,5-difluorophenyl)-2-(3-fluoropyrrolidin-1-yl)pyridin-3-yl)carbamoyl)piperidine-1-carboxylate

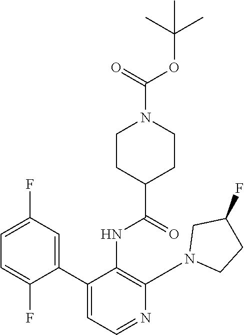 [SMILES: CC(C)(C)OC(=O)N1CCC(C(=O)Nc2c(-c3cc(F)ccc3F)ccnc2N2CC[C@H](F)C2)CC1]

To a solution of (S)-4-(2,5-difluorophenyl)-2-(3-fluoropyrrolidin-1-yl)pyridin-3-amine (0.200 g, 0.682 mmol) and 1-(tert-butoxycarbonyl)piperidine-4-carboxylic acid (0.188 g, 0.818 mmol) in tetrahydrofuran (4 mL) was added 2,4,6-tripropyl-1,3,5,2,4,6-trioxatriphosphinane 2,4,6-trioxide (0.651 g, 1.020 mmol, 50% purity in ethyl acetate) and diisopropylethylamine (0.176 g, 1.36 mmol). The mixture was stirred at 70° C. for 16 h. The reaction mixture was concentrated under reduced pressure. Purification of the residue by reversed phase column chromatography, eluting with aqueous formic acid (0.1%) in acetonitrile, afforded the title compound as a colorless solid (0.120 g, 35% yield): ¹H NMR (400 MHz, Methanol-d₄) δ 38.12 (d, J=5.2 Hz, 1H), 7.23-7.18 (m, 2H), 7.06-7.02 (m, 1H), 6.69 (d, J=4.8 Hz, 1H), 5.41-5.27 (m, 1H), 3.99-3.89 (m, 4H), 3.80-3.73 (m, 2H), 2.91-2.85 (m, 1H), 2.74 (s, 2H), 2.43-2.36 (m, 1H), 2.32-2.01 (m, 2H), 1.91-1.87 (m, 1H), 1.55-1.51 (m, 2H), 1.46 (s, 9H).

### Step 2. Preparation of (S)-N-(4-(2,5-difluorophenyl)-2-(3-fluoro pyrrolidin-1-yl)pyridin-3-yl)piperidine-4-carboxamide trifluoroacetate

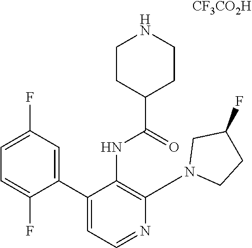 [SMILES: O=C(Nc1c(-c2cc(F)ccc2F)ccnc1N1CC[C@H](F)C1)C1CCNCC1 ; "CF3CO2H"]

To a solution of (S)-tert-butyl 4-((4-(2,5-difluorophenyl)-2-(3-fluoropyrrolidin-1-yl)pyridin-3-yl)carbamoyl) piperidine-1-carboxylate (0.100 g, 0.198 mmol) in dichloromethane (1 mL) was added trifluoroacetic acid (3.85 g, 33.7 mmol). The mixture was stirred at 20° C. for 1 h. The reaction mixture was concentrated under reduced pressure. Purification of the residue by reversed phase column chromatography, eluting with aqueous formic acid (0.1%) in acetonitrile, afforded the title compound as a colorless solid (0.130 g, crude): ¹H NMR (400 MHz, Methanol-d₄) δ 8.52 (s, 1H), 8.13 (d, J=4.8 Hz, 1H), 7.24-7.15 (m, 2H), 7.04 (s, 1H), 6.67 (d, J=5.2 Hz, 1H), 5.39-5.25 (m, 1H), 3.91-3.65 (m, 4H), 3.27-3.25 (m, 2H), 2.93 (s, 2H), 2.59-2.53 (m, 1H), 2.32-2.02 (m, 2H), 1.71-1.55 (m, 4H).

### Step 3. Preparation of (S)-N-(4-(2,5-difluorophenyl)-2-(3-fluoropyrrolidin-1-yl)pyridin-3-yl)-1-methylpiperidine-4-carboxamide

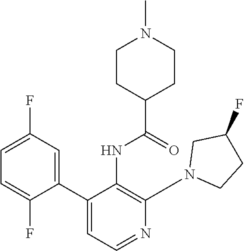 [SMILES: CN1CCC(C(=O)Nc2c(-c3cc(F)ccc3F)ccnc2N2CC[C@H](F)C2)CC1]

To a solution of (S)-N-(4-(2,5-difluorophenyl)-2-(3-fluoropyrrolidin-1-yl)pyridin-3-yl)piperidine-4-carboxamide trifluoroacetate (0.110 g, 0.212 mmol) in formic acid (4 mL) was added formaldehyde (1.09 g, 13.4 mmol, 37% purity in water). The mixture was stirred at 90° C. for 5 h. The reaction mixture was cooled to 20° C. The reaction mixture was concentrated under reduced pressure. Purification of the residue by preparative reverse phase HPLC, eluting with 27-57% aqueous ammonium hydroxide (0.05%) in acetonitrile afforded the title compound as a colorless solid (0.0127 g, 13% yield): ¹H NMR (400 MHz, Methanol-d₄) δ 8.10 (d, J=5.2 Hz, 1H), 7.22-7.12 (m, 2H), 7.04-6.99 (m, 1H), 6.65 (d, J=4.8 Hz, 1H), 5.38-5.24 (m, 1H), 3.90-3.66 (m, 4H), 2.82-2.79 (m, 2H), 2.31-2.26 (m, 1H), 2.23 (s, 3H), 2.19-1.95 (m, 4H), 1.51 (s, 4H); MS (ES+) m/z 419.3 (M+1).

### Example 17

### Synthesis of N-(4-(2,5-difluorophenyl)-2-((S)-3-fluoropyrrolidin-1-yl)pyridin-3-yl)-1-methylpiperidine-3-carboxamide

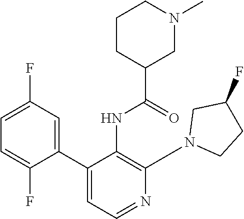 [SMILES: CN1CCCC(C(=O)Nc2c(-c3cc(F)ccc3F)ccnc2N2CC[C@H](F)C2)C1]

### Step 1. Preparation of tert-butyl 3-((4-(2,5-difluorophenyl)-2-((S)-3-fluoropyrrolidin-1-yl)pyridin-3-yl)carbamoyl)piperidine-1-carboxylate

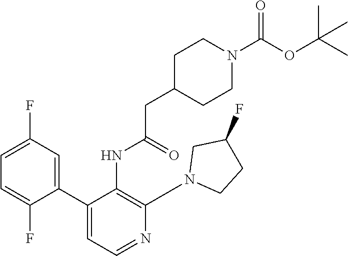 [SMILES: CC(C)(C)OC(=O)N1CCC(CC(=O)Nc2c(-c3cc(F)ccc3F)ccnc2N2CC[C@H](F)C2)CC1]

Following the procedure as reported for Example 16, step 1, replacing 1-(tert-butoxycarbonyl)piperidine-4-carboxylic acid with 1-(tert-butoxycarbonyl)piperidine-3-carboxylic acid, the title compound was isolated as a colorless solid (0.120 g, 35% yield): ¹H NMR (400 MHz, Methanol-d₄) δ 8.11 (d, J=4.8 Hz, 1H), 7.26-7.15 (m, 2H), 7.04-7.00 (m, 1H), 6.66 (d, J=5.2 Hz, 1H), 5.39-5.25 (m, 1H), 3.90-3.72 (m, 4H), 3.68 (s, 1H), 2.63 (s, 2H), 2.35-2.24 (m, 2H), 2.22-1.99 (m, 2H), 1.74-1.66 (m, 1H), 1.61-1.59 (m, 1H), 1.45 (s, 9H), 1.39-1.29 (m, 2H).

### Step 2. Preparation of N-(4-(2,5-difluorophenyl)-2-((S)-3-fluoropyrrolidin-1-yl)pyridin-3-yl)piperidine-3-carboxamide trifluoroacetate

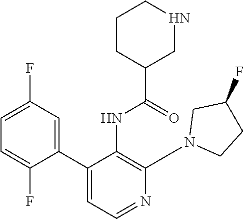 [SMILES: O=C(Nc1c(-c2cc(F)ccc2F)ccnc1N1CC[C@H](F)C1)C1CCCNC1]

Following the procedure as reported for Example 16, step 2, replacing (S)-tert-butyl 4-((4-(2,5-difluorophenyl)-2-(3-fluoropyrrolidin-1-yl)pyridin-3-yl)carbamoyl) piperidine-1-carboxylate with tert-butyl 3-((4-(2,5-difluorophenyl)-2-((S)-3-fluoropyrrolidin-1-yl)pyridin-3-yl)carbamoyl)piperidine-1-carboxylate, the title compound was isolated as a colorless solid (0.050 g crude): ¹H NMR (400 MHz, Methanol-d₄) δ 8.45 (s, 1H), 8.15 (d, J=5.0 Hz, 1H), 7.29-7.16 (m, 2H), 7.08 (d, J=2.4 Hz, 1H), 6.70 (d, J=4.8 Hz, 1H), 5.45-5.23 (m, 1H), 3.98-3.60 (m, 4H), 3.18-2.95 (m, 4H), 2.80 (s, 1H), 2.36-2.00 (m, 2H), 1.79 (d, J=3.6 Hz, 1H), 1.68-1.34 (m, 3H).

### Step 3. Preparation of N-(4-(2,5-difluorophenyl)-2-((S)-3-fluoropyrrolidin-1-yl)pyridin-3-yl)-1methylpiperidine-3-carboxamide

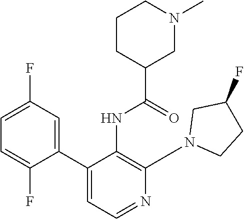 [SMILES: CN1CCCC(C(=O)Nc2c(-c3cc(F)ccc3F)ccnc2N2CC[C@H](F)C2)C1]

Following the procedure as reported for Example 16, step 3, replacing (S)-N-(4-(2,5-difluorophenyl)-2-(3-fluoropyrrolidin-1-yl)pyridin-3-yl)piperidine-4-carboxamide trifluoroacetate with N-(4-(2,5-difluorophenyl)-2-((S)-3-fluoropyrrolidin-1-yl)pyridin-3-yl)piperidine-3-carboxamide trifluoroacetate, the title compound was isolated as a colorless solid (0.0156 g, 30% yield): ¹H NMR (400 MHz, Methanol-d₄) δ8.10 (d, J=5.2 Hz, 1H), 7.24-7.15 (m, 2H), 7.04-6.99 (m, 1H), 6.65 (d, J=5.2 Hz, 1H), 5.41-5.23 (m, 1H), 3.90-3.61 (m, 4H), 2.71 (d, J=11.2 Hz, 1H), 2.59-2.38 (m, 2H), 2.32-2.23 (m, 1H), 2.21 (s, 3H), 2.18-1.97 (m, 1H), 1.92-1.87 (m, 2H), 1.62-1.44 (m, 3H), 1.20-1.1 (m, 1H); MS (ES+) m/z 419.3 (M+1).

### Example 18

### (1r,4S)-N-(4-(2,5-Difluorophenyl)-6-((S)-3-fluoropyrrolidin-1-yl)pyrimidin-5-yl)-4-methoxycyclohexane-1-carboxamide

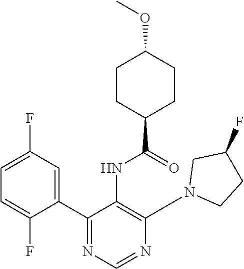 [SMILES: CO[C@H]1CC[C@H](C(=O)Nc2c(-c3cc(F)ccc3F)ncnc2N2CC[C@H](F)C2)CC1]

### Step 1. (S)-4-chloro-6-(3-fluoropyrrolidin-1-yl)pyrimidin-5-amine

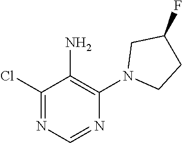 [SMILES: Nc1c(Cl)ncnc1N1CC[C@H](F)C1]

To a mixture of 4,6-dichloropyrimidin-5-amine (1.00 g, 6.10 mmol) in ethanol (10.0 mL) was added triethylamine (1.23 g, 12.2 mmol) and (S)-fluoropyrrolidine hydrochloride (0.766 g, 6.10 mmol). The reaction mixture was stirred at 80° C. for 12 h. After cooling to ambient temperature, the mixture was concentrated in vacuo. Purification of the residue by column chromatography, using 20% ethyl acetate in petroleum ether as eluent, afforded the title compound as a yellow oil (1.00 g, 76% yield): ¹H NMR (400 MHz, CD₃OD) δ7.84 (s, 1H), 5.41-5.26 (m, 1H), 4.05-3.84 (m, 4H), 2.34-2.23 (m, 1H), 2.20-2.03 (m, 1H).

### Step 2. Preparation of (1r,4r)-4-methoxycyclohexane-1-carbonyl chloride

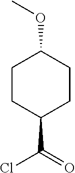

To a mixture of (1r, 4r)-4-methoxycyclohexanecarboxylic acid (0.100 g, 0.632 mmol) in thionyl chloride (3.28 g, 27.6 mmol) was added N,N-dimethylformamide (0.005 g, 0.06 mmol). The reaction mixture was stirred at 80° C. for 1 h. After cooling to ambient temperature, the mixture was concentrated in vacuo to afford a colorless solid which was used in the following step without further purification.

### Step 3. Preparation of (1r,4S)-N-(4-chloro-6-((S)-3-fluoropyrrolidin-1-yl)pyrimidin-5-yl)-4-methoxycyclohexane-1-carboxamide

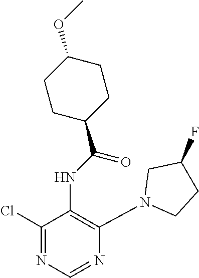 [SMILES: CO[C@H]1CC[C@H](C(=O)Nc2c(Cl)ncnc2N2CC[C@H](F)C2)CC1]

To a mixture of (S)-4-chloro-6-(3-fluoropyrrolidin-1-yl)pyrimidin-5-amine (0.110 g, 0.508 mmol) in dichloromethane (4.00 mL) was added pyridine (0.490 g, 6.19 mmol) and (1r,4r)-4-methoxycyclohexane-1-carbonyl chloride (0.110 g, 0.623 mmol). The reaction mixture was stirred at ambient temperature for 12 h. The mixture was concentrated in vacuo. Purification of the residue by column chromatography, using 50% ethyl acetate in petroleum ether as eluent,, afforded the title compound as a yellow solid (0.080 g, 43% yield): ¹H NMR (400 MHz, CD₃OD) δ 8.21 (s, 1H), 5.40-5.22 (m, 1H), 4.04-3.64 (m, 4H), 3.37 (s, 3H), 2.47-2.38 (m, 1H), 2.34-2.24 (m, 1H), 2.21-2.09 (m, 4H), 2.06-1.96 (m, 2H), 1.67-1.52 (m, 2H), 1.32-1.20 (m, 2H).

### Step 4. Preparation of (S)-2-chloro-N-(4-(2,5-difluorophenyl)-2-(3-fluoropyrrolidin-1-yl)pyridin-3-yl)pyrimidine-5-carboxamide formic acid salt

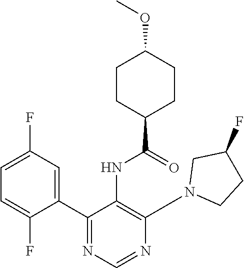 [SMILES: CO[C@H]1CC[C@H](C(=O)Nc2c(-c3cc(F)ccc3F)ncnc2N2CC[C@H](F)C2)CC1]

To a mixture of (1r,4S)-N-(4-chloro-6-((S)-3-fluoropyrrolidin-1-yl)pyrimidin-5-yl)-4-methoxycyclohexane-1-carboxamide (0.600 g, 0.168 mmol) in dioxane (4.00 mL) and water (0.800 mL) was added (2,5-difluorophenyl)boronic acid (0.053 g, 0.336 mmol), dichloro 1,1′-bis(diphenylphosphino)ferrocene palladium (II) dichloromethane (0.012 g, 0.017 mmol), and potassium carbonate (0.070 g, 0.500 mmol) and the mixture was purged with nitrogen for 10 minutes. The reaction mixture was stirred at 80° C. for 2 h. After cooling to ambient temperature, the mixture was filtered through a bed of diatomaceous earth (i.e., Celite®) and concentrated in vacuo. Purification of the residue by reverse-phase column chromatography, using a gradient of 16 to 46% acetonitrile in water containing 0.05% ammonium hydroxide as eluent, afforded the title compound as a colorless solid (0.008 g, 11% yield): ¹H NMR (400 MHz, CD₃OD) δ 8.47 (s, 1H), 7.25-7.22 (m, 2H), 7.11-7.07 (m, 1H), 5.42-5.28 (m, 1H), 4.13-3.65 (m, 4H), 3.31 (s, 3H), 3.12-3.04 (m, 1H), 2.37-2.27 (m, 1H), 2.23-1.99 (m, 4H), 1.82-1.66 (m, 1H), 1.42-1.29 (m, 2H), 1.11-1.08 (m, 3H); MS (ES+) m/z 435.0 (M+1).

### Example 19

### (S)-N-(4-(2,5-difluorophenyl)-6-(3-fluoropyrrolidin-1-yl)pyrimidin-5-yl)-1-isopropyl-1H-pyrazole-4-carboxamide

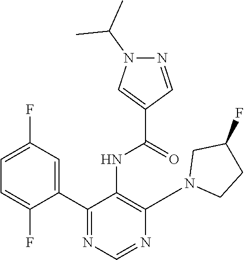 [SMILES: CC(C)n1cc(C(=O)Nc2c(-c3cc(F)ccc3F)ncnc2N2CC[C@H](F)C2)cn1]

### Step 1. Preparation of 1-isopropyl-1H-pyrazole-4-carbonyl chloride hydrochloride

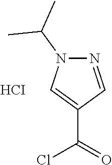

To a solution of thionyl chloride (7.38 g, 62.0 mmol) was added 1-isopropyl-1H-pyrazole-4-carboxylic acid (0.450 g, 2.92 mmol). The reaction mixture was stirred at 80° C. for 3 h. After cooling to ambient temperature, the mixture was concentrated in vacuo to afford a yellow oil which was used in the following step without further purification.

### Step 2. Preparation of (S)-N-(4-chloro-6-(3-fluoropyrrolidin-1-yl)pyrimidin-5-yl)-1-isopropyl-1H-pyrazole-4-carboxamide

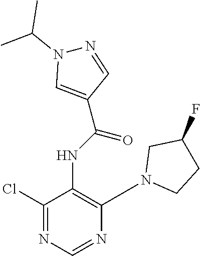 [SMILES: CC(C)n1cc(C(=O)Nc2c(Cl)ncnc2N2CC[C@H](F)C2)cn1]

To a mixture of (S)-4-chloro-6-(3-fluoropyrrolidin-1-yl)pyrimidin-5-amine (0.400 g, 1.85 mmol) in dichloromethane (8.00 mL) was added sodium tert-butoxide (0.889 g, 9.25 mmol) and 1-isopropyl-1H-pyrazole-4-carbonyl chloride hydrochloride (0.479 g, 2.77 mmol). The reaction mixture was stirred at ambient temperature for 12 h. The mixture was concentrated in vacuo. Purification of the residue by column chromatography, using 50% ethyl acetate in petroleum ether as eluent, afforded the title compound as a yellow solid (0.300 g, 46% yield): ¹H NMR (400 MHz, DMSO-d₆) δ 9.73 (d, J=6.0 Hz, 1H), 8.36 (d, J=5.6 Hz, 1H), 8.30 (d, J=2.8 Hz, 1H), 8.02 (s, 1H), 5.43-5.30 (m, 1H), 4.56 (m, 1H), 3.95-3.88 (m, 1H), 3.81-3.69 (m, 2H), 3.62-3.49 (m, 1H), 2.24-2.01 (m, 2H), 1.45 (d, J=6.4 Hz, 6H).

### Step 3. Preparation of (S)-N-(4-(2,5-difluorophenyl)-6-(3-fluoropyrrolidin-1-yl)pyrimidin-5-yl)-1-isopropyl-1H-pyrazole-4-carboxamide

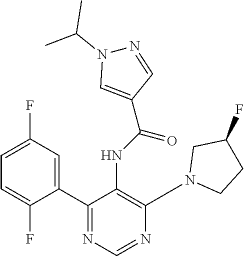 [SMILES: CC(C)n1cc(C(=O)Nc2c(-c3cc(F)ccc3F)ncnc2N2CC[C@H](F)C2)cn1]

To a mixture of (S)-N-(4-chloro-6-(3-fluoropyrrolidin-1-yl)pyrimidin-5-yl)-1-isopropyl-1H-pyrazole-4-carboxamide (0.050 g, 0.142 mmol) in dioxane (5.00 mL) and water (0.100 mL) was added (2,5-difluorophenyl)boronic acid (0.034 g, 0.213 mmol), 20 dichloro 1,1′-bis(diphenylphosphino)ferrocene palladium (II) dichloromethane (0.010 g, 0.014 mmol), and potassium carbonate (0.039 g, 0.28 mmol) and the mixture was purged with nitrogen for 10 minutes. The reaction mixture was stirred at 80° C. for 2 h. After cooling to ambient temperature, the mixture was concentrated in vacuo. Purification of the residue by reverse-phase column chromatography, using a gradient of 18 to 48% acetonitrile in water containing 0.23% formic acid as eluent, and then 15 to 45% acetonitrile in water containing 0.05% ammonium hydroxide as eluent, afforded the title compound as a colorless solid (0.013 g, 21% yield): ¹H NMR (400 MHz, CD₃OD) δ 8.50 (s, 1H), 8.07 (s, 1H), 7.82 (s, 1H), 7.17-7.14 (m, 3H), 5.37-5.24 (m, 1H), 4.56-4.49 (m, 1H), 4.06-3.73 (m, 4H), 2.33-1.99 (m, 2H), 1.48 (d, J=6.8 Hz, 6H); MS (ES+) m/z 431.0 (M+1).

### Example 20

### Synthesis of 1-cyclobutyl-N-(4-(2,5-difluorophenyl)-6-(3,3-difluoropyrrolidin-1-yl)pyrimidin-5-yl)-1H-pyrazole-4-carboxamide

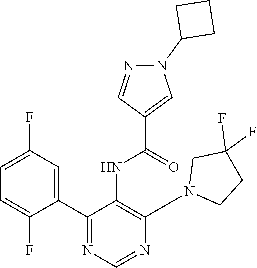 [SMILES: O=C(Nc1c(-c2cc(F)ccc2F)ncnc1N1CCC(F)(F)C1)c1cnn(C2CCC2)c1]

### Step 1. Preparation of 1-cyclobutyl-N-(4,6-dichloropyrimidin-5-yl)-1H-pyrazole-4-carboxamide

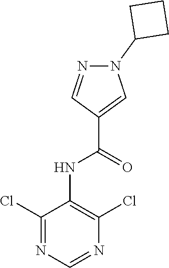 [SMILES: O=C(Nc1c(Cl)ncnc1Cl)c1cnn(C2CCC2)c1]

To a solution of 1-cyclobutylpyrazole-4-carboxylic acid (0.300 g, 1.81 mmol) in dichloromethane (2 mL) was added oxalyl dichloride (0.252 g, 1.99 mmol) and dimethyl formamide (0.0132 g, 0.181 mmol) dropwise at 0° C. The solution was stirred at 20° C. for 2 h. The solution was evaporated under reduced pressure to give 1-cyclobutylpyrazole-4-carbonyl chloride (0.300 g, 1.62 mmol) as a light yellow oil. To a solution of 4,6-dichloropyrimidin-5-amine (0.200 g, 1.22 mmol) in tetrahydrofuran (2 mL) was added sodium hydride (0.244 g, 6.10 mmol, 60% purity) in portions at 0° C. The mixture was stirred at 0° C. for 1 h then a solution of 1-cyclobutylpyrazole-4-carbonyl chloride (0.248 mg, 1.34 mmol) in tetrahydrofuran (1 mL) was added dropwise at 0 0° C. The mixture was stirred at 20° C. for 1 h. The mixture was poured into water (20 mL). The mixture was extracted with ethyl acetate (3×20 mL). The combined organic layers were washed with brine (10 mL), dried over anhydrous sodium sulfate, filtered and evaporated under reduced pressure. The residue was purified by column chromatography eluting with 40% ethyl acetate in petroleum ether to give 1-cyclobutyl-N-(4,6-dichloropyrimidin-5-yl)-pyrazole-4-carboxamide as a white solid (0.230 g, 57% yield): ¹H NMR (400 MHz, CDCl₃) δ 8.72 (s, 1H), 8.08 (s, 1H), 7.96 (s, 1H), 7.37 (s, 1H), 4.89-4.76 (m, 1H), 2.65-2.47 (m, 4H), 2.01-1.85 (m, 2H).

### Step 2. Preparation of N-(4-chloro-6-(3,3-difluoropyrrolidin-1-yl)-pyrimidin-5-yl)-1-cyclobutyl-1H-pyrazole-4-carboxamide

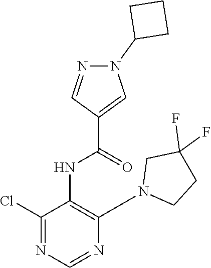 [SMILES: O=C(Nc1c(Cl)ncnc1N1CCC(F)(F)C1)c1cnn(C2CCC2)c1]

To a mixture of 1-cyclobutyl-N-(4,6-dichloropyrimidin-5-yl)pyrazole-4-carboxamide (0.100 g, 0.320 mmol) and 3,3-difluoropyrrolidine hydrochloride (0.0920 g, 0.641 mmol) in ethanol (2 mL) was added N,N-diisopropylethylamine (0.207 g, 1.60 mmol) dropwise at 20° C. The solution was stirred at 70° C. for 2 h. The mixture was cooled to 20° C. and poured into water (20 mL). The mixture was extracted with ethyl acetate (3×20 mL). The combined organic layers were washed with brine (20 mL), dried over anhydrous sodium sulfate, filtered and the filtrate was evaporated under reduced pressure. Purification of the residue by preparative reverse phase HPLC, eluting with 27-57% aqueous ammonium formate (10 mM) in acetonitrile, afforded the title compound as a colorless solid as a white solid (0.0900 g, 73% yield): ¹H NMR (400 MHz, CDCl₃) δ 8.34 (s, 1H), 8.06 (s, 1H), 7.92 (s, 1H), 7.06-6.91 (m, 1H), 4.89-4.76 (m, 1H), 4.10-3.83 (m, 4H), 2.67-2.48 (m, 4H), 2.45-2.30 (m, 2H), 2.03-1.84 (m, 2H).

### Step 3. Preparation of 1-cyclobutyl-N-(4-(2,5-difluorophenyl)-6-(3,3-difluoropyrrolidin-1-yl)pyrimidin-5-yl)-1H-pyrazole-4-carboxamide

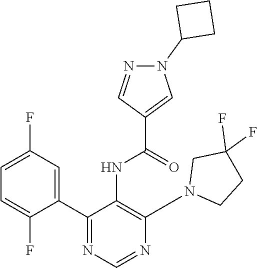 [SMILES: O=C(Nc1c(-c2cc(F)ccc2F)ncnc1N1CCC(F)(F)C1)c1cnn(C2CCC2)c1]

To a solution of N-(4-chloro-6-(3,3-difluoropyrrolidin-1-yl)pyrimidin-5-yl)-1-cyclobutyl-1H-pyrazole-4-carboxamide (0.0400 g, 0.105 mmol) and (2,5-difluorophenyl)boronic acid (0.0330 g, 0.209 mmol) in dioxane (1.5 mL) and water (0.15 mL) was added potassium carbonate (0.0433 g, 0.313 mmol) and 1,1′-bis(diphenylphosphino)ferrocene-palladium(II) dichloride dichloromethane complex (0.00853 g, 0.0105 mmol) in one portion at 20 0° C. The mixture was stirred at 95° C. under a nitrogen atmosphere for 2 h. The mixture was cooled to 20° C. and poured into water (10 mL). The mixture was extracted with ethyl acetate (3×10 mL). The combined organic layers were washed with brine (10 mL), dried over anhydrous sodium sulfate, filtered and the filtrate was evaporated under reduced pressure.

Purification of the residue by preparative reverse phase HPLC, eluting with 24-54% aqueous ammonium formate (10 mM) in acetonitrile, afforded the title compound as a colorless solid as an off-white solid (0.0341 g, 98% purity):¹H NMR (400 MHz, DMSO-d₆) δ 9.55 (s, 1H), 8.58 (s, 1H), 8.21 (s, 1H), 7.86 (s, 1H), 7.33-7.20 (m, 2H), 7.19-7.11 (m, 1H), 4.90-4.75 (m, 1H), 4.26-3.61 (m, 4H), 2.49-2.30 (m, 6H), 1.85-1.68 (m, 2H); MS (ES+) m/z=461.1 (M+1).

### Example 21

### Synthesis of 1-cyclobutyl-N-(4-(3,3-difluoropyrrolidin-1-yl)-6-phenylpyrimidin-5-yl)-1H-pyrazole-4-carboxamide

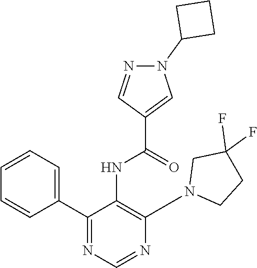 [SMILES: O=C(Nc1c(-c2ccccc2)ncnc1N1CCC(F)(F)C1)c1cnn(C2CCC2)c1]

Following the procedure as reported for Example 1, step 3, replacing 2,4-difluorophenyl boronic acid with phenyl boronic acid, the title compound was isolated as a colorless solid (0.0278 g, 49% yield): ¹H NMR (400 MHz, DMSO-d₆) δ 9.59 (s, 1H), 8.57 (d, J=0.8 Hz, 1H), 8.23 (s, 1H), 7.90 (s, 1H), 7.68-7.52 (m, 2H), 7.44-7.29 (m, 3H), 4.94-4.75 (m, 1H), 4.17-4.02 (m, 1H), 4.01-3.80 (m, 2H), 3.79-3.67 (m, 1H), 2.49-2.30 (m, 6H), 1.84-1.70 (m, 2H); MS (ES+) m/z=425.2 (M+1).

### Example 22

### Synthesis of (R)-1-cyclobutyl-N-(4-phenyl-6-(2-(trifluoromethyl)pyrrolidin-1-yl)pyrimidin-5-yl)-1H-pyrazole-4-carboxamide

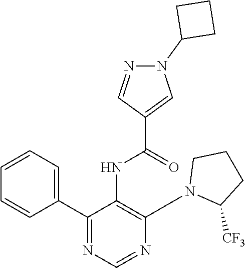 [SMILES: O=C(Nc1c(-c2ccccc2)ncnc1N1CCC[C@@H]1C(F)(F)F)c1cnn(C2CCC2)c1]

### Step 1. Preparation of (R)-N-(4-chloro-6-(2-(trifluoromethyl)-pyrrolidin-1-yl)pyrimidin-5-yl)-1-cyclobutyl-1H-pyrazole-4-carboxamide

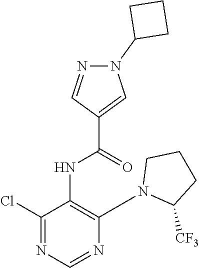 [SMILES: O=C(Nc1c(Cl)ncnc1N1CCC[C@@H]1C(F)(F)F)c1cnn(C2CCC2)c1]

To a solution of 1-cyclobutyl-N-(4,6-dichloropyrimidin-5-yl)pyrazole-4-carboxamide (0.200 g, 0.640 mmol) and diisopropylethylamine (0.414 g, 3.20 mmol) in butan-1-ol (1 mL) was added (R)-2-(trifluoromethyl)pyrrolidine (0.225 g, 1.28 mmol) in one portion at 20 0° C. The solution was stirred at 90° C. for 12 h. The mixture was cooled to 20° C. and poured into water (10 mL). The mixture was extracted with ethyl acetate (3×20 mL). The combined organic fractions were washed with brine (10 mL), dried over anhydrous sodium sulfate, filtered and the filtrate was evaporated under reduced pressure to give the title compound as a colorless solid (0.250 g, 94% yield): ¹H NMR (400 MHz, CDCl₃) δ 8.37 (s, 1H), 8.08 (s, 1H), 7.95 (s, 1H), 7.25 (s, 1H), 5.58-5.34 (m, 1H), 4.96-4.69 (m, 1H), 2.62-2.49 (m, 4H), 2.23-1.82 (m, 8H).

### Step 2. Preparation of (R)-1-cyclobutyl-N-(4-phenyl-6-(2-(trifluoromethyl)pyrrolidin-1-yl)pyrimidin-5-yl)-1H-pyrazole-4-carboxamide

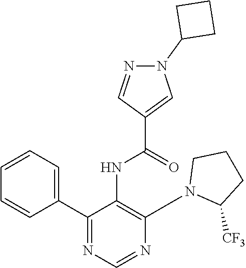 [SMILES: O=C(Nc1c(-c2ccccc2)ncnc1N1CCC[C@@H]1C(F)(F)F)c1cnn(C2CCC2)c1]

To a solution of (R)-N-(4-chloro-6-(2-(trifluoromethyl)pyrrolidin-1-yl)pyrimidin-5-yl)-1-cyclobutyl-1H-pyrazole-4-carboxamide (0.250 g, 0.603 mmol) and phenylboronic acid (0.147 g, 1.21 mmol) in dioxane (10 mL) and water (0.1 mL) was added potassium carbonate (0.250 g, 1.81 mmol) and 1,1′-bis(diphenylphosphino)ferrocene-palladium(II) 20 dichloride dichloromethane complex (0.0492 g, 0.0603 mmol) in one portion at 20° C. The mixture was stirred at 95° C. under nitrogen atmosphere for 2 h. The mixture was cooled to 20° C. and poured into water (20 mL). The mixture was extracted with ethyl acetate (3×30 mL). The combined organic layers were washed with brine (20 mL), dried over anhydrous sodium sulfate, filtered and evaporated under reduced pressure. Purification by preparative reverse phase HPLC, eluting with 37-67% aqueous ammonium formate (10 mM) in acetonitrile afforded the title compound as an off-white solide (0.142 g, 50% yield): ¹H NMR (400 MHz, DMSO-d₆) δ 9.81-9.47 (m, 1H), 8.61 (s, 1H), 8.23 (d, J=16.8 Hz, 1H), 7.91 (s, 1H), 7.62 (d, J=5.2 Hz, 2H), 7.50-7.24 (m, 3H), 5.80-5.50 (m, 1H), 4.83 (t, J=7.6 Hz, 1H), 4.02-3.42 (m, 2H), 2.49-2.29 (m, 4H), 2.18-1.91 (m, 4H), 1.86-1.67 (m, 2H); MS (ES+) m/z 457.1 (M+1)

### Example 23

### Synthesis of N-(4-(3,3-difluoropyrrolidin-1-yl)-6-phenylpyrimidin-5-yl)-6-isopropylnicotinamide

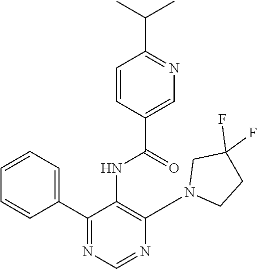 [SMILES: CC(C)c1ccc(C(=O)Nc2c(-c3ccccc3)ncnc2N2CCC(F)(F)C2)cn1]

### Step 1. Preparation of 4-chloro-6-(3,3-difluoropyrrolidin-1-yl)-pyrimidin-5-amine

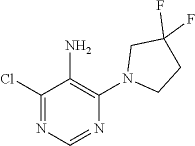 [SMILES: Nc1c(Cl)ncnc1N1CCC(F)(F)C1]

A mixture of 4,6-dichloropyrimidin-5-amine (4.00 g, 24.4 mmol), 3,3-difluoropyrrolidine hydrochloride (10.5 g, 73.2 mmol) and triethylamine (14.8 g, 146.4 mmol) in ethanol (80 mL) was stirred at 70° C. for 12 h. After being cooled to ambient temperature, the mixture was concentrated in vacuo. The residue was purified by reverse phase chromatography, eluting with 0.1% aqueous ammonium hydroxide to afford the title compound as a light-yellow solid (5.30 g, 93% yield);¹H NMR (400 MHz, DMSO-d₆) δ 7.90 (s, 1H), 4.92 (s, 2H), 3.98 (t, J=13.6 Hz, 2H), 3.79 (t, J=7.2 Hz, 2H), 2.44 (td, J=7.2, 13.6 Hz, 2H).

### Step 2. Preparation of N-(4-chloro-6-(3,3-difluoropyrrolidin-1-yl)-pyrimidin-5-yl)-6-isopropylnicotinamide

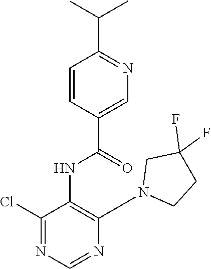 [SMILES: CC(C)c1ccc(C(=O)Nc2c(Cl)ncnc2N2CCC(F)(F)C2)cn1]

To a mixture of 6-isopropylnicotinic acid (0.300 g, 1.82 mmol) in tetrahydrofuran (7.5 mL) was added N,N-diisopropylethylamine (1.17 g, 9.08 mmol) and 2-chloro-1-methyl-pyridin-1-ium iodide (0.557 g, 2.18 mmol). The mixture was then stirred at 25° C. for 2 h. To this mixture was added 4-chloro-6-(3,3-difluoropyrrolidin-1-yl)pyrimidin-5-amine (0.852 g, 3.63 mmol). The resulting mixture was stirred at 60° C. for 12 h under a nitrogen atmosphere. The mixture was concentrated in vacuo. Purification by preparative reverse phase HPLC, eluting with 30-52% aqueous formic acid (0.225%) in acetonitrile afforded the title compound as a colorless solid (0.110 g, 16% yield); ¹H NMR (400 MHz, DMSO-d₆) δ 10.33 (s, 1H), 9.06 (d, J=1.6 Hz, 1H), 8.38 (s, 1H), 8.24 (dd, J=2.4, 8.4 Hz, 1H), 7.49 (d, J=8.0 Hz, 1H), 4.10 (q, J=12.4 Hz, 1H), 4.03-3.83 (m, 2H), 3.72 (td, J=7.6, 11.2 Hz, 1H), 3.12 (td, J=6.8, 13.8 Hz, 1H), 2.46 (d, J=6.4 Hz, 2H), 1.27 (d, J=7.2 Hz, 6H).

### Step 3. Preparation of of N-(4-(3,3-difluoropyrrolidin-1-yl)-6-phenylpyrimidin-5-yl)-6-isopropylnicotinamide

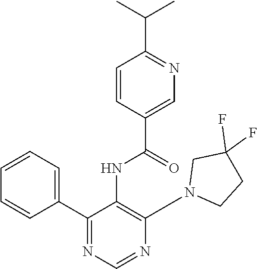 [SMILES: CC(C)c1ccc(C(=O)Nc2c(-c3ccccc3)ncnc2N2CCC(F)(F)C2)cn1]

A mixture of N-(4-chloro-6-(3,3-difluoropyrrolidin-1-yl)pyrimidin-5-yl)-6-isopropylnicotinamide (0.0500 g, 0.131 mmol), phenylboronic acid (0.0240 g, 0.196 mmol), potassium carbonate (0.0543 g, 0.393 mmol) and [1,1-bis(diphenylphosphino)ferrocene]-dichloropalladium(II) (0.0958 g, 0.0131 mmol) in 1,4-dioxane (1 mL) and water (0.12 mL) was stirred at 80° C. for 12 h under a nitrogen atmosphere. The mixture was diluted with ethyl acetate (5 mL) and filtered.

The filtrate was concentrated in vacuo. Purification of the residue by preparative reverse phase HPLC, eluting with 25-45% aqueous formic acid (0.225%) in acetonitrile afforded the title compound as a colorless solid (0.0299 g, 53% yield): ¹H NMR (400 MHz, MeOD) δ 8.69 (dd, J=0.4, 2.4 Hz, 1H), 8.55 (s, 1H), 8.00 (dd, J=2.4, 8.0 Hz, 1H), 7.54-7.48 (m, 2H), 7.45-7.37 (m, 4H), 4.17-3.84 (m, 4H), 3.11 (td, J=6.8, 13.8 Hz, 1H), 2.53-2.38 (m, 2H), 1.30 (d, J=7.2 Hz, 6H); MS (ES+) m/z 424.0 (M+1).

### Example 24

### Synthesis of N-(4-(3,3-difluoropyrrolidin-1-yl)-6-(2-fluorophenyl)pyrimidin-5-yl)-6-isopropylnicotinamide

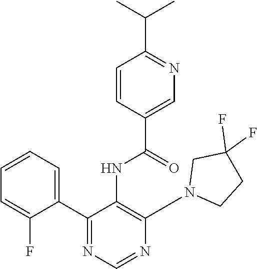 [SMILES: CC(C)c1ccc(C(=O)Nc2c(-c3ccccc3F)ncnc2N2CCC(F)(F)C2)cn1]

Following the procedure as reported for Example 23, step 3, replacing phenyl boronic acid with 2-F-phenyl boronic acid, the title compound was isolated as a colorless solid (0.0306 g, 57% yield): ¹H NMR (400 MHz, MeOD) δ 8.66 (s, 1H), 8.57 (s, 1H), 7.99-7.98 (m, 1H), 7.45-7.39 (m, 3H), 7.24-7.20 (m, 2H), 4.19-3.89 (m, 4H), 3.14-3.07 (m, 1H), 2.49-2.42 (m, 2H), 1.29 (d, J=7.2, 6H); MS (ES+) m/z 441.2 (M+1).

### Example 25

### Synthesis of N-(4-(2,5-difluorophenyl)-6-(3,3-difluoropyrrolidin-1-yl)pyrimidin-5-yl)-6-isopropylnicotinamide

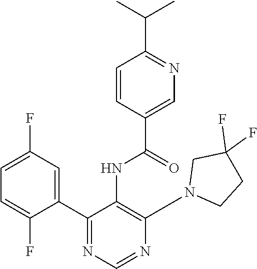 [SMILES: CC(C)c1ccc(C(=O)Nc2c(-c3cc(F)ccc3F)ncnc2N2CCC(F)(F)C2)cn1]

Following the procedure as reported for Example 23, step 3, replacing phenyl boronic acid with 2,5-di-F-phenyl boronic acid, the title compound was isolated as a colorless solid (0.0279 g, 52% yield): ¹H NMR (400 MHz, MeOD) δ 8.72 (s, 1H), 8.59 (s, 1H), 8.04-8.00 (m, 1H), 7.42 (d, J=8.4 Hz, 1H), 7.20-7.15 (m, 3H), 4.08-3.94 (m, 4H), 3.15-3.10 (m, 1H), 2.49-2.42 (m, 2H), 1.30 (d, J=7.2, 6H); MS (ES+) m/z 459.2 (M+1).

### Example 26

### Synthesis of N-(4-(3,3-difluoropyrrolidin-1-yl)-6-phenylpyrimidin-5-yl)-2-isopropylpyrimidine-5-carboxamide

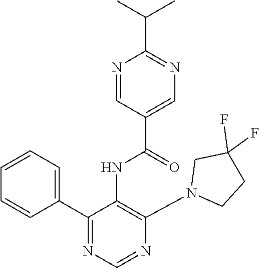 [SMILES: CC(C)c1ncc(C(=O)Nc2c(-c3ccccc3)ncnc2N2CCC(F)(F)C2)cn1]

### Step 1. Preparation of N-(4-chloro-6-(3,3-difluoropyrrolidin-1-yl)pyrimidin-5-yl)-6-isopropylnicotinamide

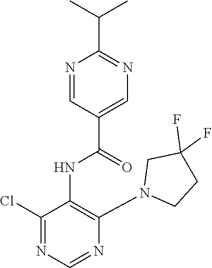 [SMILES: CC(C)c1ncc(C(=O)Nc2c(Cl)ncnc2N2CCC(F)(F)C2)cn1]

To a mixture of 2-isopropylpyrimidine-5-carboxylic acid (0.0390 g, 0.234 mmol) in tetrahydrofuran (1 mL) was added pyridine (0.169 g, 2.13 mmol), 2-chloro-1-methyl-pyridin-1-ium iodide (0.163 g, 0.639 mmol) and 4-chloro-6-(3,3-difluoropyrrolidin-1-yl)pyrimidin-5-amine (0.0500 g, 0.213 mmol). The mixture was stirred at 60° C. for 12 h. The mixture was quenched with saturated ammonium chloride (1 mL). The mixture was extracted with ethyl acetate (3×5 mL) and concentrated in vacuo. Purification of the residue by preparative reverse phase HPLC, eluting with 30-50% aqueous formic acid (0.225%) in acetonitrile afforded the title compound as a yellow solid (0.0180 g, 20% yield): ¹H NMR (400 MHz, MeOD-d₄) δ 9.23 (s, 2H), 8.33 (s, 1H), 4.14-3.79 (m, 4H), 3.36-3.33 (m, 1H), 2.52-2.36 (m, 2H), 1.39 (d, J=6.8 Hz, 6H).

### Step 2. Preparation of N-(4-(3,3-difluoropyrrolidin-1-yl)-6-phenylpyrimidin-5-yl)-2-isopropylpyrimidine-5-carboxamide formate salt

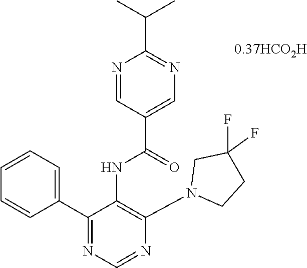 [SMILES: CC(C)c1ncc(C(=O)Nc2c(-c3ccccc3)ncnc2N2CCC(F)(F)C2)cn1]

A mixture of N-(4-chloro-6-(3,3-difluoropyrrolidin-1-yl)pyrimidin-5-yl)-6-isopropylnicotinamide (0.0170 g, 0.0444 mmol), phenylboronic acid (0.00812 g, 0.0666 mmol), potassium carbonate (0.0184 g, 0.133 mmol) and [1,1-bis(diphenylphosphino)ferrocene]-dichloropalladium(II) (0.00325 g, 0.00444 mmol) in 1,4-dioxane (0.4 mL) and water (0.06 mL) was stirred at 80° C. for 12 h under a nitrogen atmosphere. The mixture was quenched by the addition of water (5 mL) and extracted with ethyl acetate (3×10 mL). The organic phases were combined, dried over sodium sulfate and filtered. The filtrate was concentrated in vacuo. Purification of the residue by preparative reverse phase HPLC, eluting with 22-42% aqueous formic acid (0.225%) in acetonitrile afforded the title compound as a colorless solid (0.0299 g, 53% yield): ¹H NMR (400 MHz, MeOD-d₄) δ 8.88 (s, 2H), 8.55 (s, 1H), 8.48 (s, 0.37H), 7.53-7.47 (m, 2H), 7.45-7.38 (m, 3H), 4.15-3.97 (m, 3H), 3.95-3.84 (m, 1H), 3.23 (td, J=7.2, 13.6 Hz, 1H), 2.55-2.37 (m, 2H), 1.33 (d, J=6.8 Hz, 6H); MS (ES+) m/z 425.1 (M+1)

### Example 27

### Synthesis of N-(4-(2,5-difluorophenyl)-6-(3,3-difluoropyrrolidin-1-yl)pyrimidin-5-yl)-2-isopropylpyrimidine-5-carboxamide

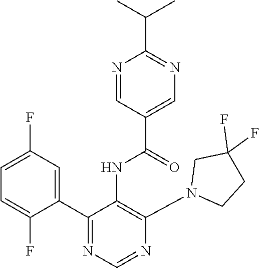 [SMILES: CC(C)c1ncc(C(=O)Nc2c(-c3cc(F)ccc3F)ncnc2N2CCC(F)(F)C2)cn1]

### Step 1. Preparation of 4-(2,5-difluorophenyl)-6-(3,3-difluoro-pyrrolidin-1-yl)pyrimidin-5-amine

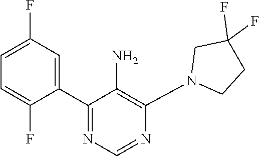 [SMILES: Nc1c(-c2cc(F)ccc2F)ncnc1N1CCC(F)(F)C1]

A mixture of chloro-6-(3,3-difluoropyrrolidin-1-yl)pyrindin-5-amine (0.500 g, 2.13 mmol), (2,5-difluoro-phenyl)boronic acid (0.505 g, 3.20 mmol), potassium carbonate (0.884 g, 6.39 mmol) and [1,1-bis(diphenyl-phosphino)ferrocene]-dichloropalladium(ii) (0.156 g, 0.213 mmol) in 1,4-dioxane (5 mL) and water (0.6 mL) was stirred at 80° C. for 12 h under a nitrogen atmosphere. The mixture was quenched by the addition of water (5 mL) and extracted with ethyl acetate (3×10 mL). The organic phase was dried over sodium sulfate and filtered. The filtrate was concentrated in vacuo. Purification of the residue by column chromatography, eluting with 10:1 ethyl acetate in petroleum ether, afforded the title compound as a yellow solid (0.350 g, 53% yield): ¹H NMR (400 MHz, CDCl₃) δ 8.39 (s, 1H), 7.27-7.24 (m, 1H), 7.21-7.11 (m, 2H), 3.98 (t, J=13.2 Hz, 2H), 3.89 (t, J=7.2 Hz, 2H), 3.41 (br s, 2H), 2.52-2.39 (m, 2H).

### Step 2. Preparation of N-(4-(2,5-difluorophenyl)-6-(3,3-difluoropyrrolidin-1-yl)pyrimidin-5-yl)-2-isopropylpyrimidine-5-carboxamide

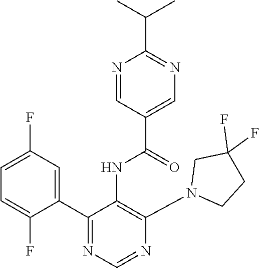 [SMILES: CC(C)c1ncc(C(=O)Nc2c(-c3cc(F)ccc3F)ncnc2N2CCC(F)(F)C2)cn1]

To a mixture of 2-isopropylpyrimidine-5-carboxylic acid (0.0681 g, 0.410 mmol) in tetrahydrofuran (1 mL) were added N-ethyl-N-isopropylpropan-2-amine (0.331 g, 2.56 mmol), 2-chloro-1-methyl-pyridin-1-ium iodide (0.262 g, 1.02 mmol) and 4-(2,5-difluorophenyl)-6-(3,3-difluoropyrrolidin-1-yl)pyrimidin-5-amine (0.800 g, 0.256 mmol). The mixture was stirred 60° C. for 12 h. After being cooled to ambient temperature, the mixture was diluted with water (0.5 mL). The filtrate was concentrated in vacuo. Purification of the residue by preparative reverse phase HPLC, eluting with 36-56% aqueous formic acid (0.225%) in acetonitrile afforded the title compound as a yellow solid (0.0552 g, 46% yield):¹H NMR (400 MHz, MeOD-d₄) δ 8.92 (s, 2H), 8.50 (s, 1H), 7.26-7.14 (m, 3H), 4.22-3.80 (m, 4H), 3.24 (td, J=6.8, 13.6 Hz, 1H), 2.55-2.38 (m, 2H), 1.34 (d, J=6.8 Hz, 6H); MS (ES+) m/z 461.2(M+1).

### Example 28

### Synthesis of N-(4-(3,3-difluoropyrrolidin-1-yl)-6-(2-fluorophenyl)pyrimidin-5-yl)-2-isopropylpyrimidine-5-carboxamide

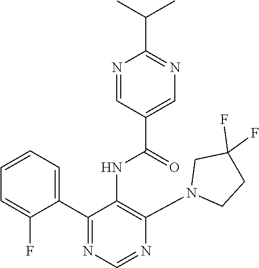 [SMILES: CC(C)c1ncc(C(=O)Nc2c(-c3ccccc3F)ncnc2N2CCC(F)(F)C2)cn1]

### Step 1. Preparation of 4-(2-fluorophenyl)-6-(3,3-difluoro-pyrrolidin-1-yl)pyrimidin-5-amine

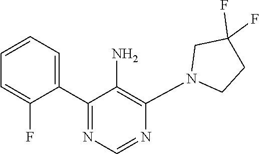 [SMILES: Nc1c(-c2ccccc2F)ncnc1N1CCC(F)(F)C1]

Following the procedure as reported for Example 26, step 1, replacing 4-(2,5-difluorophenyl)-6-(3,3-difluoropyrrolidin-1-yl)pyrimidin-5-amine with 4-(2-fluorophenyl)-6-(3,3-difluoropyrrolidin-1-yl)pyrimidin-5-amine, the title compound was used directly in step 2.

### Step 2. Synthesis of N-(4-(3,3-difluoropyrrolidin-1-yl)-6-(2-fluorophenyl)pyrimidin-5-yl)-2-isopropylpyrimidine-5-carboxamide

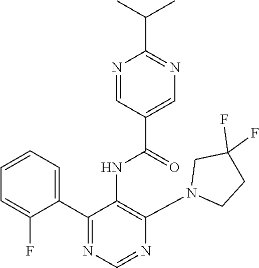 [SMILES: CC(C)c1ncc(C(=O)Nc2c(-c3ccccc3F)ncnc2N2CCC(F)(F)C2)cn1]

Following the procedure as reported for Example 26, step 2, replacing 2,5-difluorophenyl boronic acid with 2-F-phenyl boronic acid, the title compound was isolated as a yellow solid (0.0596 g, 49% yield):¹H NMR (400 MHz, MeOD-d₄) δ 8.86 (s, 2H), 8.58 (s, 1H), 7.47-7.40 (m, 2H), 7.26-7.20 (m, 2H), 4.07-3.90 (m, 4H), 3.23 (td, J=6.8, 13.5 Hz, 1H), 2.52-2.41 (m, 2H), 1.32 (d, J=6.8 Hz, 6H); MS (ES+) m/z 443.1 (M+1).

### Example 29

### Synthesis of (S)-N-(4-(2,5-difluorophenyl)-6-(3-fluoropyrrolidin-1-yl)pyrimidin-5-yl)-1-methyl-1H-imidazole-4-carboxamide

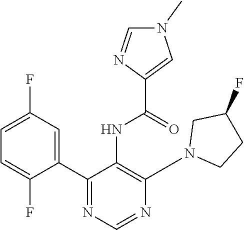 [SMILES: Cn1cnc(C(=O)Nc2c(-c3cc(F)ccc3F)ncnc2N2CC[C@H](F)C2)c1]

### Step 1. Preparation of 1-methyl-1H-imidazole-4-carbonyl chloride

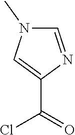

A slurry of the 1-methyl-1H-imidazole-4-carboxylic acid (0.500 g, 3.96 mmol) in dry dichloromethane (10 mL) at 20° C. was treated with dropwise addition of oxalyl chloride (0.870 g, 6.85 mmol) and N,N-dimethylformamide (0.0290 g, 0.396 mmol). The reaction bubbled immediately and the slurry was stirred at 20° C. for 1 h. The reaction mixture was concentrated under reduced pressure to give 1-methyl-1H-imidazole-4-carbonyl chloride as a colorless solid (0.800 g, crude, hydrochloride).

### Step 2. Preparation of (S)-N-(4-chloro-6-(3-fluoropyrrolidin-1-yl)pyrimidin-5-yl)-1-methyl-1H-imidazole-4-carboxamide

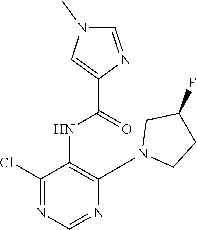 [SMILES: Cn1cnc(C(=O)Nc2c(Cl)ncnc2N2CC[C@H](F)C2)c1]

To a solution of (S)-4-chloro-6-(3-fluoropyrrolidin-1-yl)pyrimidin-5-amine (0.400 g, 1.85 mmol) in dichloromethane (10 mL) was added sodium tert-butoxide (0.887 g, 9.23 mmol) and 1-methyl-1H-imidazole-4-carbonyl chloride (0.501 g, 2.77 mmol, hydrochloride). The mixture was stirred at 20° C. for 12 h. The reaction mixture was concentrated under reduced pressure. The residue was purified by column chromatography on silica gel, eluting with ethyl acetate (50%) in petroleum ether, to afford the title compound as a red oil (0.220 g, 37% yield):¹H NMR (400 MHz, DMSO-d₆) δ 9.69 (s, 1H), 8.26 (s, 1H), 7.78 (s, 1H), 7.75 (s, 1H), 5.40-5.27 (m, 1H), 3.98-3.84 (m, 2H), 3.72 (s, 3H), 3.57-3.44 (m, 2H), 2.20-2.07 (m, 2H).

### Step 3. Preparation of (S)-N-(4-(2,5-difluorophenyl)-6-(3-fluoropyrrolidin-1-yl)pyrimidin-5-yl)-1-methyl-1H-imidazole-4-carboxamide

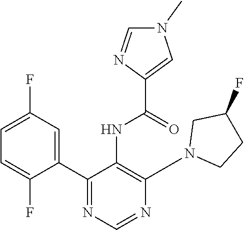 [SMILES: Cn1cnc(C(=O)Nc2c(-c3cc(F)ccc3F)ncnc2N2CC[C@H](F)C2)c1]

A mixture of (S)-N-(4-chloro-6-(3-fluoropyrrolidin-1-yl)pyrimidin-5-yl)-1-methyl-1H-imidazole-4-carboxamide (0.0500 g, 0.154 mmol), (2,5-difluorophenyl)boronic acid (0.0486 g, 0.309 mmol), [1,1-bis(diphenylphosphino) ferrocene]dichloropalladium(II) (0.0113 g, 0.0154 mmol), potassium carbonate (0.0638 g, 0.462 mmol) in dioxane (1.5 mL) and water (0.3 mL) was degassed and purged with nitrogen 3 times. The mixture was stirred at 100° C. for 16 h under a nitrogen atmosphere. The reaction mixture was concentrated under reduced pressure. Purification of the residue by preparative reverse phase HPLC, eluting with 4-34% aqueous formic acid (0.225%) in acetonitrile afforded the title compound as a yellow solid (0.00330 g, 5% yield): ¹H NMR (400 MHz, Methanol-d₄) δ8.49 (s, 1H), 7.59 (d, J=2.4 Hz, 2H), 7.21-7.17 (m, 1H), 7.13-7.09 (m, 2H), 5.35-5.21 (m, 1H), 4.04-3.99 (m, 2H), 3.91-3.76 (m, 2H), 3.73 (s, 3H), 2.30-1.98 (m, 2H); MS (ES+) m/z 403.1 (M+1).

### Example 30

### Synthesis of (S)-N-(4-(2,5-difluorophenyl)-2-(3-fluoropyrrolidin-1-yl)pyridin-3-yl)-2-(4-isopropylphenyl)acetamide

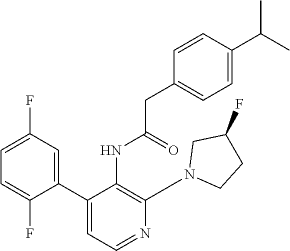 [SMILES: CC(C)c1ccc(CC(=O)Nc2c(-c3cc(F)ccc3F)ccnc2N2CC[C@H](F)C2)cc1]

To a solution of (S)-4-(2,5-difluorophenyl)-2-(3-fluoropyrrolidin-1-yl)pyridin-3-amine (0.100 g, 0.341 mmol) and 2-(4-isopropylphenyl)acetic acid (0.0912 g, 0.511 mmol) in tetrahydrofuran (5 mL) was added 2,4,6-tripropyl-1,3,5,2,4,6-trioxatriphosphinane 2,4,6-trioxide (0.434 g, 0.682 mmol, 50% purity in ethyl acetate) and N,N-diisopropylethylamine (0.132 g, 1.02 mmol). The mixture was stirred at 70° C. for 12 h. The reaction mixture was cooled to ambient temperature, poured into water (20 mL), and then extracted with ethyl acetate (3×10 mL). The combined organic layers were washed with brine (3×30 mL), dried over sodium sulfate, filtered and concentrated under reduced pressure. Purification of the residue by preparative reverse phase HPLC, eluting with 42-81% aqueous formic acid (0.225%) in acetonitrile afforded the title compound as a colorless solid (0.0608 g, 39% yield): ¹H NMR (400 MHz, Methanol-d₄) δ 8.07 (d, J=4.8 Hz, 1H), 7.10-7.08 (m, 2H), 7.06-7.03 (m, 2H), 6.98-6.96 (m, 2H), 6.95-6.91 (m, 1H), 6.61 (d, J=5.2 Hz, 1H), 5.28-5.13 (m, 1H), 3.78-3.54 (m, 4H), 3.38 (d, J=2.0 Hz, 2H), 2.91-2.84 (m, 1H), 2.22-1.93 (m, 2H), 1.24 (d, J=6.8 Hz, 6H); MS (ES+) m/z 454.1 (M+1).

### Example 31

### Synthesis of (R)-N-(4-(2,5-difluorophenyl)-6-(2-(trifluoromethyl)pyrrolidin-1-yl)pyrimidin-5-yl)-2-isopropylpyrimidine-5-carboxamide

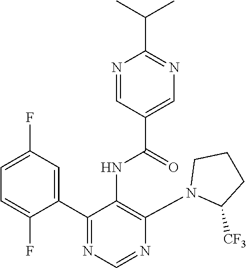 [SMILES: CC(C)c1ncc(C(=O)Nc2c(-c3cc(F)ccc3F)ncnc2N2CCC[C@@H]2C(F)(F)F)cn1]

### Step 1. Preparation of (R)-4-chloro-5-nitro-6-(2-(trifluoromethyl) pyrrolidin-1-yl)pyrimidine

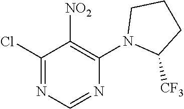 [SMILES: O=[N+]([O-])c1c(Cl)ncnc1N1CCC[C@@H]1C(F)(F)F]

To a solution of 4,6-dichloro-5-nitropyrimidine (0.500 g, 2.58 mmol) in acetonitrile (10 mL) was added potassium carbonate (1.78 g, 12.9 mmol) and (R)-2-(trifluoromethyl)pyrrolidine hydrochloride (0.460 g, 2.62 mmol). The mixture was stirred at 20° C. for 2 h. The reaction mixture was filtered and concentrated under reduced pressure. The residue was purified by column chromatography on silica gel eluting with a 10:1 mixture of petroleum ether and ethyl acetate to give (R)-4-chloro-5-nitro-6-(2-(trifluoromethyl)pyrrolidin-1-yl)pyrimidine as a yellow oil (0.400 g, 52% yield): ¹H NMR (400 MHz, CDCl₃) δ 8.46 (s, 1H), 5.58-5.51 (m, 1H), 3.63-3.58 (m, 1H), 3.34-3.28 (m, 1H), 2.30-2.19 (m, 2H), 2.18-2.06 (m, 2H).

### Step 2. Preparation of (R)-4-chloro-6-(2-(trifluoromethyl) pyrrolidin-1-yl)pyrimidin-5-amine

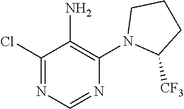 [SMILES: Nc1c(Cl)ncnc1N1CCC[C@@H]1C(F)(F)F]

To a solution of (R)-4-chloro-5-nitro-6-(2-(trifluoromethyl)pyrrolidin-1-yl)pyrimidine (0.400 g, 1.35 mmol) in methanol (20 mL) and water (2 mL) was added zinc (0.440 g, 6.73 mmol) and ammonium chloride (0.720 g, 13.6 mmol). The mixture was stirred at 60° C. for 12 h. The reaction mixture was cooled to ambient temperature. The mixture was filtered and concentrated under reduced pressure. The residue was dissolve in ethyl acetate (100 mL), washed with brine (3×100 mL), dried over sodium sulfate, filtered and concentrated under reduced pressure. The residue was purified by column chromatography on silica gel, eluting with a 1:1 mixture of petroleum ether and ethyl acetate, to give (R)-4-chloro-6-(2-(trifluoromethyl)pyrrolidin-1-yl)pyrimidin-5-amine as a yellow oil. (0.200 g, 56% yield): MS (ES+) m/z=267.1 (M+1).

### Step 3. Preparation of (R)-4-(2,5-difluorophenyl)-6-(2-(trifluoromethyl)pyrrolidin-1-yl)pyrimidin-5-amine

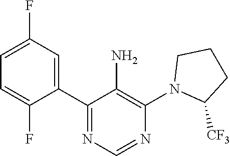 [SMILES: Nc1c(-c2cc(F)ccc2F)ncnc1N1CCC[C@@H]1C(F)(F)F]

To a solution of (R)-4-chloro-6-(2-(trifluoromethyl)pyrrolidin-1-yl)pyrimidin-5-amine (0.150 g, 0.563 mmol) and (2,5-difluorophenyl)boronic acid (0.107 g, 0.678 mmol) in dioxane (6 mL) and water (0.6 mL) was added potassium carbonate (0.156 g, 1.13 mmol) in water (0.6 mL) and 1,1′-bis(diphenylphosphino) ferrocene-palladium(II) dichloride dichloromethane complex (0.046 g, 0.0563 mmol). The mixture was stirred at 90° C. for 1.5 h. The reaction mixture was concentrated under reduced pressure. The residue was purified by column chromatography on silica gel, eluting with a 10:1 mixture of petroleum ether and ethyl acetate, to give (R)-4-(2,5-difluorophenyl)-6-(2-(trifluoromethyl)pyrrolidin-1-yl)pyrimidin-5-amine as a yellow oil (0.140 g, 72% yield): ¹H NMR (400 MHz, CDCl₃) δ 8.39 (s, 1H), 7.32-7.28 (m, 1H), 7.21-7.12 (m, 2H), 5.64-5.55 (m, 1H), 3.41-3.35 (m, 1H), 2.35-2.24 (m, 1H), 2.19-2.07 (m, 2H), 1.97-1.89 (m, 2H).

### Step 4. Preparation of (R)-N-(4-(2,5-difluorophenyl)-6-(2-(trifluoromethyl)pyrrolidin-1-yl)pyrimidin-5-yl)-2-isopropylpyrimidine-5-carboxamide

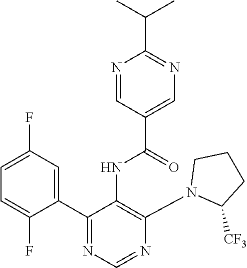 [SMILES: CC(C)c1ncc(C(=O)Nc2c(-c3cc(F)ccc3F)ncnc2N2CCC[C@@H]2C(F)(F)F)cn1]

To a solution of (R)-4-(2,5-difluorophenyl)-6-(2-(trifluoromethyl)pyrrolidin-1-yl)pyrimidin-5-amine (0.050 g, 0.145 mmol) and 2-isopropylpyrimidine-5-carboxylic acid (0.039 g, 0.235 mmol) in tetrahydrofuran (1 mL) was added 2-chloro-1-methylpyridinium iodide (0.149 g, 0.583 mmol) and diisopropylethylamine (0.188 g, 1.45 mmol). The mixture was stirred at 65° C. for 12 h. The reaction mixture was concentrated under reduced pressure. Purification of the residue by preparative reverse phase HPLC, eluting with 42-72% aqueous formic acid (0.225%) in acetonitrile afforded the title compound. To a solution of the overacylation byproduct in methanol (2 mL) was added lithium hydroxide (0.5 M, 0.5 mL) and the mixture was stirred at 20° C. for 12 h. The mixture was concentrated under reduced pressure. Purification of the residue by preparative reverse phase HPLC, eluting with 42-72% aqueous formic acid (0.225%) in acetonitrile afforded the title compound as a colorless solid (0.0087 g, 2% yield): ¹H NMR (400 MHz, CDCl₃) δ 8.94 (s, 2H), 8.72 (s, 1H), 7.72 (d, J=3.6 Hz, 1H), 7.35-7.27 (m, 1H), 7.12-7.07 (m, 2H), 5.62-5.55 (m, 1H), 3.85-3.80 (m, 1H), 3.65-3.59 (m, 1H), 3.32-3.25 (m, 1H), 2.20-2.09 (m, 3H), 2.06-2.01 (m, 1H), 1.37 (d, J=6.8 Hz, 6H); MS (ES+) m/z=493.1 (M+1).

### Example 32

### Synthesis of N-[2-(3,3-difluoropyrrolidin-1-yl)-4-(2-pyridyl)-3-pyridyl]-2-isopropyl-pyrimidine-5-carboxamide

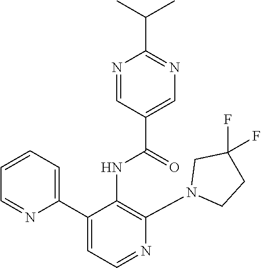 [SMILES: CC(C)c1ncc(C(=O)Nc2c(-c3ccccn3)ccnc2N2CCC(F)(F)C2)cn1]

### Step 1. Preparation of potassium 2-(3,3-difluoropyrrolidin-1-yl)-4-iodo-pyridine-3-carboxylate

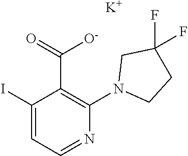 [SMILES: [K+] ; O=C([O-])c1c(I)ccnc1N1CCC(F)(F)C1]

To a mixture of 2-fluoro-4-iodo-pyridine-3-carboxylic acid (12.5 g, 46.8 mmol) and potassium carbonate (12.9 g, 93.6 mmol) in N,N-dimethylformamide (500 mL) was added 3,3-difluoropyrrolidin-1-ium chloride (6.72 g, 46.8 mmol), and the mixture was stirred at 85° C. for 20 h. After cooling to ambient temperature, the mixture was diluted with ethyl acetate (2500 mL) and filtered through diatomaceous earth (i.e., Celite®) washing with ethyl acetate (500 mL), and the filtrate was concentrated in vacuo. The residue was stirred in a mixture of ethyl acetate (20 mL) and diethyl ether (250 mL) for 30 minutes and the solid was filtered washing with diethyl ether (50 mL). The residue was dried in vacuo, to afford the title compound as a brown solid (9.57 g, 47% yield): ¹H NMR (400 MHz, DMSO-d₆) δ 7.45 (d, J=5.2 Hz, 1H), 6.94 (d, J=5.2 Hz, 1H), 3.95 (t, J=13.9 Hz, 2H), 3.73 (t, J=7.3 Hz, 2H), 2.38 (tt, J=14.4, 7.3 Hz, 2H); MS (ES+) m/z 355.2 (M+1).

### Step 2. Preparation of [2-(3,3-difluoropyrrolidin-1-yl)-4-iodo-3-pyridyl]ammonium chloride

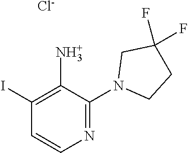 [SMILES: [Cl-] ; [NH3+]c1c(I)ccnc1N1CCC(F)(F)C1]

To a mixture of potassium 2-(3,3-difluoropyrrolidin-1-yl)-4-iodo-pyridine-3-carboxylate (7.50 g, 19.1 mmol) and triethylamine (6.66 mL, 47.8 mmol) in N-methylpyrollidone (190 mL) was added diphenylphosphoryl azide (6.17 mL, 28.7 mmol), and the mixture was stirred at 95° C. for 3 h. After cooling to ambient temperature, the mixture was diluted with saturated aqueous sodium bicarbonate (1000 mL), and the aqueous phase was extracted with ethyl acetate (3×1000 mL).

The organic phase was washed with brine (1000 mL), dried over anhydrous sodium sulfate, filtered and concentrated in vacuo. The residue was purified by column chromatography, eluting with a gradient of 0-40% of ethyl acetate in hexanes. The residue was diluted with diethyl ether (50 mL), and hydrochloric acid (2 M solution in diethyl ether, 11.5 mL, 22.9 mmol) was added. Filtration, washing with diethyl ether (5×100 mL), and drying the residue in vacuo afforded the title compound as a pink solid (4.80 g, 66%): ¹H NMR (400 MHz, DMSO-d₆) δ 7.44-7.33 (m, 1H), 7.25 (d, J=5.6 Hz, 1H), 6.09 (s, 3H), 3.85 (t, J=13.5 Hz, 2H), 3.59 (dd, J=14.5, 7.4 Hz, 2H), 2.59-2.41 (m, 2H); MS (ES+) m/z 326.3 (M+1).

### Step 3. Preparation of N-[2-(3,3-difluoropyrrolidin-1-yl)-4-iodo-3-pyridyl]-2-isopropyl-pyrimidine 5-carboxamide

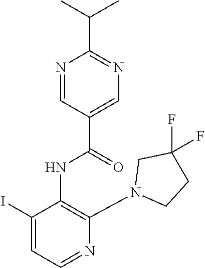 [SMILES: CC(C)c1ncc(C(=O)Nc2c(I)ccnc2N2CCC(F)(F)C2)cn1]

To a solution of 2-(3,3-difluoropyrrolidin-1-yl)-4-iodo-pyridin-3-amine hydrochloride (2.00 g, 5.53 mmol), 2-isopropylpyrimidine-5-carboxylic acid (1.37 g, 8.30 mmol), and 2-chloro-1-methyl-pyridin-1-ium iodide (5.65 g, 22.1 mmol) in tetrahydrofuran (50.0 mL) was added N,N-diisopropylethylamine (3.79 mL, 22.1 mmol), and the mixture was stirred at 65° C. for 20 h. After cooling to ambient temperature, the mixture was diluted with ethyl acetate (50 mL), and the organic phase was washed with saturated aqueous sodium bicarbonate (50 mL). The organic phase was dried over anhydrous sodium sulfate, filtered, and concentrated in vacuo. The residue was diluted with methanol (100 mL), filtered washing with methanol (50 mL) and the solid was concentrated in vacuo to afford the title compound as a colorless solid (1.73 g, 66% yield): ¹H NMR (400 MHz, CDCl₃) δ 9.20 (s, 2H), 7.78 (d, J=4.8 Hz, 1H), 7.37 (s, 1H), 7.28 (d, J=5.1 Hz, 1H), 3.92-3.73 (m, 4H), 3.37-3.26 (m, 1H), 2.42-2.30 (m, 2H), 1.41 (d, J=6.9 Hz, 6H); MS (ES+) m/z 474.4 (M+1).

### Step 4. Preparation of N-[2-(3,3-difluoropyrrolidin-1-yl)-4-(2-pyridyl)-3-pyridyl]-2-isopropyl-pyrimidine-5-carboxamide

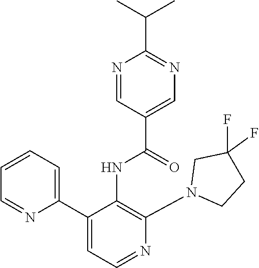 [SMILES: CC(C)c1ncc(C(=O)Nc2c(-c3ccccn3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.100 g, 0.211 mmol), 2-(1,1,1-tributylstannyl)pyridine (0.0820 mL, 0.254 mmol), and 2-dicyclohexylphosphino-2′,4′,6′-triisopropylbiphenyl (0.0550 mg, 0.127 mmol) in degassed 1,4-dioxane (2.00 mL) was added palladium acetate (0.014 mg, 0.063 mmol), and the mixture was stirred at 100° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by column chromatography, eluting with a gradient of 0-10% of methanol in dichloromethane, followed by preparative reverse phase HPLC, eluting with a gradient of 38-48% acetonitrile in water containing 10 mM of ammonium formate, afforded the title compound as a solid (0.0100 g, 11% yield): ¹H NMR (400 MHz; DMSO-d₆) δ 10.33 (s, 1H), 9.00 (s, 2H), 8.65-8.55 (m, 1H), 8.12 (s, 1H), 7.84-7.59 (m, 2H), 7.34-7.21 (m, 1H), 6.97 (d, J=5.0 Hz, 1H), 3.91 (t, J=13.6 Hz, 2H), 3.75 (t, J=7.1 Hz, 2H), 3.17 (dt, J=13.8, 6.9 Hz, 1H), 2.40 (tt, J=14.2, 7.1 Hz, 2H), 1.28 (d, J=6.9 Hz, 6H); MS (ES+) m/z 425.3 (M+1).

### Example 33

### Synthesis of N-[2-(3,3-difluoropyrrolidin-1-yl)-4-(2-methylpyrazol-3-yl)-3-pyridyl]-2-isopropyl-pyrimidine-5-carboxamide

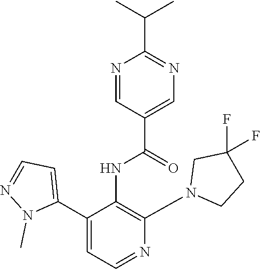 [SMILES: CC(C)c1ncc(C(=O)Nc2c(-c3ccnn3C)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 mg, 0.106 mmol), (1-methyl-1H-pyrazol-5-yl)boronic acid (0.0200 mg, 0.158 mmol), and potassium carbonate (0.0360 mg, 0.264 mmol) in degassed 1,4-dioxane (1.00 mL) and water (0.300 mL) was added [1,1′ bis(diphenylphosphino)ferrocene]dichloropalladium(II), complex with dichloromethane (0.0250 g, 0.0310 mmol) and the mixture was stirred at 100° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo.

Purification of the residue by column chromatography, eluting with a gradient of 0-10% of methanol in dichloromethane, followed by preparative reverse phase HPLC, eluting with a gradient of 30-40% acetonitrile in water containing 10 mM of ammonium formate, afforded the title compound as a solid (0.0200 g, 44% yield): ¹H NMR (400 MHz; DMSO-d₆) δ 10.19 (s, 1H), 8.96 (s, 2H), 8.12 (s, 1H), 7.30 (s, 1H), 6.74 (s, 1H), 6.21 (s, 1H), 4.03-3.83 (m, 2H), 3.74 (dd, J=15.6, 6.2 Hz, 2H), 3.68 (s, 3H), 3.14 (ddd, J=15.5, 9.5, 5.7 Hz, 1H), 2.44-2.24 (m, 2H), 1.28 (d, J=6.9 Hz, 6H); MS (ES+) m/z 428.3 (M+1).

### Example 34

### Synthesis of N-[2-(3,3-difluoropyrrolidin-1-yl)-4-(1H-indazol-5-yl)-3-pyridyl]-2-isopropyl-pyrimidine-5-carboxamide

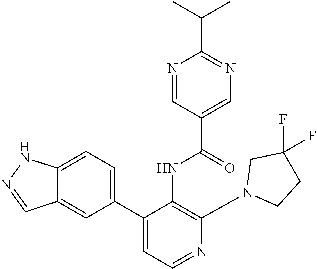 [SMILES: CC(C)c1ncc(C(=O)Nc2c(-c3ccc4[nH]ncc4c3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), 1H-indazol-5-ylboronic acid (0.0340 g, 0.200 mmol), and potassium carbonate (0.0350 g, 0.253 mmol) in 1,4-dioxane (1.00 mL) and water (0.300 mL) was added [1,1′-bis(diphenylphosphino)ferrocene]dichloropalladium(II), complex with dichloromethane (0.0250 g, 0.0306 mmol), and the mixture was stirred at 100° C. for 2 h. After cooling to ambient temperature, the mixture was diluted with ethyl acetate (10 mL). The mixture was filtered through a bed of diatomaceous earth (i.e., Celite®). The solid was washed with ethyl acetate (50 mL), and the filtrate concentrated in vacuo. Purification of the residue by column chromatography, eluting with a gradient of 0-10% of methanol in dichloromethane, followed by preparative reverse phase HPLC, eluting with a gradient of 31-41% acetonitrile in water containing 10 mM of ammonium formate, afforded the title compound as a colorless solid (0.0220 g, 47% yield): ¹H NMR (400 MHz; DMSO-d₆) δ 13.04 (s, 1H), 10.12 (s, 1H), 8.86 (s, 2H), 8.14 (d, J=5.0 Hz, 1H), 8.03 (s, 1H), 7.73 (s, 1H), 7.48 (d, J=8.7 Hz, 1H), 7.34 (dd, J=8.6, 1.6 Hz, 1H), 6.80 (d, J=5.0 Hz, 1H), 3.97-3.57 (m, 4H), 3.15-3.01 (m, 1H), 2.43-2.31 (m, 2H), 1.19 (d, J=6.9 Hz, 6H); MS (ES+) m/z 464.3 (M+1).

### Example 35

### Synthesis of N-(2-(3,3-difluoropyrrolidin-1-yl)-4-((dimethyl(oxo)-lambda6-sulfaneylidene)amino)pyridin-3-yl)-2-isopropylpyrimidine-5-carboxamide

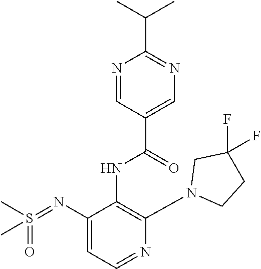 [SMILES: CC(C)c1ncc(C(=O)Nc2c(N=S(C)(C)=O)ccnc2N2CCC(F)(F)C2)cn1]

To a solution of 2-[2-(3,3-difluoropyrrolidin-1-yl)-4-iodo-3-pyridyl]-1,4,6,7-tetrahydropyrano[3,4-d]imidazole (0.0600 g, 0.127 mmol), imino-dimethyl-oxo-/ambda6-sulfane (0.0120 mL, 0.152 mmol), tris(dibenzylideneacetone)dipalladium(0) (0.0120 g, 0.0130 mmol) and 4,5-bis(diphenylphosphino)-9,9-dimethylxanthene (0.0150 g, 0.0260 mmol) in 1,4-dioxane (1.20 mL) was added sodium tert-butoxide (0.0240 g, 0.0250 mmol), and the mixture was stirred at 100° C. for 1 h. After cooling to ambient temperature, the mixture was diluted with ethyl acetate (50 mL). The mixture was filtered through a bed of diatomaceous earth (i.e., Celite®). The solid was washed with ethyl acetate (50 mL) and methanol (30 mL), and the filtrate was concentrated in vacuo. Purification of the residue by column chromatography, eluting with a gradient of 0-20% of methanol in dichloromethane, followed by preparative reverse phase HPLC, eluting with a gradient of 18-28% acetonitrile in water containing 10 mM of ammonium formate, afforded the title compound as a colorless solid (0.0300 g, 47% yield): ¹H NMR (400 MHz, DMSO-d₆) δ 59.65 (s, 1H), 9.19 (s, 2H), 7.84 (d, J=5.5 Hz, 1H), 6.64 (d, J=5.4 Hz, 1H), 3.91-3.61 (m, 4H), 3.26-3.19 (m, 1H), 3.17 (s, 6H), 2.38 (ddd, J=21.4, 14.2, 7.1 Hz, 2H), 1.31 (d, J=6.9 Hz, 6H); MS (ES+) m/z 439.3 (M+1).

### Example 36

### Synthesis of N-[4-(1H-benzimidazol-5-yl)-2-(3,3-difluoropyrrolidin-1-yl)-3-pyridyl]-2-isopropyl-pyrimidine-5-carboxamide

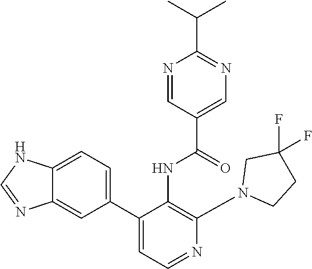 [SMILES: CC(C)c1ncc(C(=O)Nc2c(-c3ccc4[nH]cnc4c3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), 1H-benzimidazol-5-ylboronic acid (0.034 0g, 0.200 mmol), and potassium carbonate (0.0350 g, 0.253 mmol) in 1,4-dioxane (1.00 mL) and water (0.300 mL) was added [1,1′-bis(diphenylphosphino)ferrocene]dichloropalladium(II), complex with dichloromethane (0.0250 g, 0.0306 mmol), and the mixture was stirred at 100° C. for 2 h. After cooling to ambient temperature, the mixture was diluted with ethyl acetate (10 mL). The mixture was filtered through a bed of diatomaceous earth (i.e., Celite®). The solid was washed with ethyl acetate (30 mL) and methanol (20 mL), and the filtrate was concentrated in vacuo. Purification of the residue by column chromatography, eluting with a gradient of 0-10% of methanol in dichloromethane, followed by preparative reverse phase HPLC, eluting with a gradient of 29-39% acetonitrile in water containing 10 mM of ammonium formate, afforded the title compound as a colorless solid (0.0180 g, 41% yield): ¹H NMR (400 MHz; DMSO-d₆) δ 512.50 (d, J=19.7 Hz, 1H), 10.16 (s, 1H), 8.91 (d, J=4.0 Hz, 2H), 8.21 (s, 1H), 8.19 (d, J=2.8 Hz, 1H), 7.74-7.61 (m, 1H), 7.52 (d, J=8.7 Hz, 1H), 7.30-7.18 (m, 1H), 6.86 (d, J=5.0 Hz, 1H), 4.12-3.59 (m, 4H), 3.20-3.08 (m, 1H), 2.42 (ddd, J=16.5, 12.0, 4.9 Hz, 2H), 1.25 (d, J=6.9 Hz, 6H); MS (ES+) m/z 464.3 (M+1).

### Example 37

### Synthesis of N-[4-(6-amino-3-pyridyl)-2-(3,3-difluoropyrrolidin-1-yl)-3-pyridyl]-2-isopropyl-pyrimidine-5-carboxamide

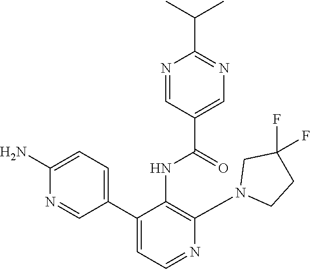 [SMILES: CC(C)c1ncc(C(=O)Nc2c(-c3ccc(N)nc3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), 5-(4,4,5,5-tetramethyl-1,3,2-dioxaborolan-2-yl)pyridin-2-amine (0.0470 g, 0.203 mmol), and potassium carbonate (0.0350 g, 0.253 mmol) in 1,4-dioxane (1.00 mL) and water (0.300 mL) was added [1,1′-bis(diphenylphosphino)ferrocene]dichloropalladium(II), complex with dichloromethane (0.025 g, 0.0306 mmol), and the mixture was stirred at 90° C. for 1 h. After cooling to ambient temperature, the mixture was diluted with ethyl acetate (10 mL). The mixture was filtered through a bed of diatomaceous earth (i.e., Celite®). The solid was washed with ethyl acetate (20 mL) and methanol (20 mL), and the filtrate was concentrated in vacuo. Purification of the residue by column chromatography, eluting with a gradient of 0-10% of methanol in dichloromethane, followed by preparative reverse phase HPLC, eluting with a gradient of 28-38% acetonitrile in water containing 10 mM of ammonium formate, afforded the title compound as a colorless solid (0.0350 g, 78% yield): ¹H NMR (400 MHz; DMSO-d₆) δ 10.11 (s, 1H), 8.98 (s, 2H), 8.09 (d, J=5.0 Hz, 1H), 7.93 (d, J=1.8 Hz, 1H), 7.39 (dd, J=8.6, 2.5 Hz, 1H), 6.73 (d, J=5.0 Hz, 1H), 6.49-6.21 (m, 1H), 6.03 (s, 2H), 4.01-3.47 (m, 4H), 3.20-3.10 (m, 1H), 2.37 (dq, J=21.7, 7.1 Hz, 2H), 1.24 (d, J=6.9 Hz, 6H); MS (ES+) m/z 440.3 (M+1).

### Example 38

### Synthesis of N-[2-(3,3-difluoropyrrolidin-1-yl)-4-(3-fluorophenyl)-3-pyridyl]-2-isopropyl-pyrimidine-5-carboxamide

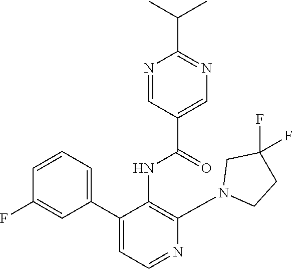 [SMILES: CC(C)c1ncc(C(=O)Nc2c(-c3cccc(F)c3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), (3-fluorophenyl)boronic acid (0.0300 g, 0.204 mmol), and potassium carbonate (0.0350 g, 0.253 mmol) in 1,4-dioxane (1.200 mL) and water (0.400 mL) was added [1,1′-bis(diphenylphosphino)ferrocene]dichloropalladium(II), complex with dichloromethane (0.0250 g, 0.0306 mmol), and the mixture was stirred at 100° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate concentrated in vacuo. Purification of the residue by column chromatography, eluting with a gradient of 0-10% of methanol in dichloromethane, followed by preparative reverse phase HPLC, eluting with a gradient of 49-59% acetonitrile in water containing 10 mM of ammonium formate, afforded the title compound as a colorless solid (0.0360 g, 81% yield): ¹H NMR (400 MHz; DMSO-d₆) δ 10.19 (s, 1H), 8.94 (s, 2H), 8.21 (d, J=5.0 Hz, 1H), 7.43 (td, J=8.0, 6.1 Hz, 1H), 7.32-7.09 (m, 3H), 6.83 (d, J=5.0 Hz, 1H), 3.83 (d, J=58.1 Hz, 4H), 3.24-3.12 (m, 1H), 2.49-2.36 (m, 2H), 1.27 (d, J=6.9 Hz, 6H); MS (ES+) m/z 442.3 (M+1).

### Example 39

### Synthesis of N-[4-(2,3-difluorophenyl)-2-(3,3-difluoropyrrolidin-1-yl)-3-pyridyl]-2-isopropyl-pyrimidine-5-carboxamide

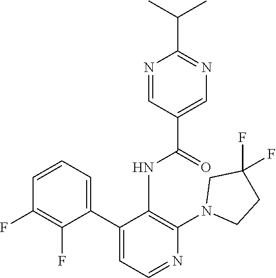 [SMILES: CC(C)c1ncc(C(=O)Nc2c(-c3cccc(F)c3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), (2,3-difluorophenyl)boronic acid (0.033 g, 0.199 mmol), and potassium carbonate (0.0350 g, 0.253 mmol) in 1,4-dioxane (1.200 mL) and water (0.400 mL) was added [1,1′-bis(diphenylphosphino)ferrocene]dichloropalladium(II), complex with dichloromethane (0.0250 g, 0.0306 mmol), and the mixture was stirred at 70° C. for 20 minutes. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate was concentrated in vacuo. Purification of the residue by column chromatography, eluting with a gradient of 0-10% of methanol in dichloromethane, followed by preparative reverse phase HPLC, eluting with a gradient of 50-60% acetonitrile in water containing 10 mM of ammonium formate, afforded the title compound as a colorless solid (0.0380 g, 82% yield): ¹H NMR (400 MHz; DMSO-d₆) δ 10.18 (s, 1H), 8.85 (s, 2H), 8.18 (d, J=4.9 Hz, 1H), 7.35 (dd, J=17.3, 9.0 Hz, 1H), 7.15 (dd, J=12.6, 8.7 Hz, 1H), 7.06 (t, J=6.1 Hz, 1H), 6.81 (d, J=4.9 Hz, 1H), 3.99-3.53 (m, 4H), 3.19-3.05 (m, 1H), 2.43-2.33 (m, 2H), 1.22 (d, J=6.9 Hz, 6H); MS (ES+) m/z 460.2 (M+1).

### Example 40

### Synthesis of N-[2-(3,3-difluoropyrrolidin-1-yl)-4-(6-fluoro-1H-indazol-5-yl)-3-pyridyl]-2-isopropyl-pyrimidine-5-carboxamide

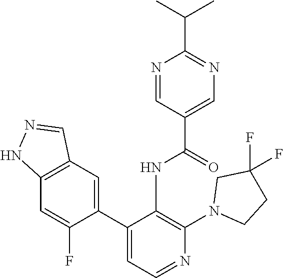 [SMILES: CC(C)c1ncc(C(=O)Nc2c(-c3cc4cn[nH]c4cc3F)ccnc2N2CCC(F)(F)C2)cn1]

### Step 1. Preparation of 6-fluoro-5-(4,4,5,5-tetramethyl-1,3,2-dioxaborolan-2-yl)-1H-indazole

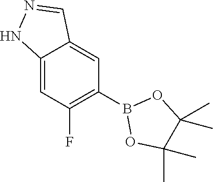 [SMILES: CC1(C)OB(c2cc3cn[nH]c3cc2F)OC1(C)C]

To a solution of 5-bromo-6-fluoro-1H-indazole (0.500 g, 2.33 mmol), 4,4,5,5-tetramethyl-2-(4,4,5,5-tetramethyl-1,3,2-dioxaborolan-2-yl)-1,3,2-dioxaborolane (1.29 g, 5.12 mmol) and [1,1′ bis(diphenylphosphino)ferrocene]dichloropalladium(II), complex with dichloromethane (0.380 g, 0.460 mmol) in 1,4-dioxane (6.00 mL) was added potassium acetate (685 g, 6.98 mmol), and the mixture was stirred at 100° C. for 20 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by column chromatography, eluting with a gradient of 0-100% of methanol in dichloromethane, afforded the title compound as a brown oil (50% pure, 0.776 g, 63% yield): ¹H NMR (300 MHz; DMSO-d₆) δ 13.12 (s, 1H), 8.10 (s, 1H), 7.80 (dd, J=8.8, 5.3 Hz, 1H), 7.00 (td, J=9.2, 2.2 Hz, 1H), 1.16 (s, 12H); MS (ES+) m/z 263.1 (M+1).

### Step 2. Preparation of N-[2-(3,3-difluoropyrrolidin-1-yl)-4-(6-fluoro-1H-indazol-5-yl)-3-pyridyl]-2-isopropyl-pyrimidine-5-carboxamide

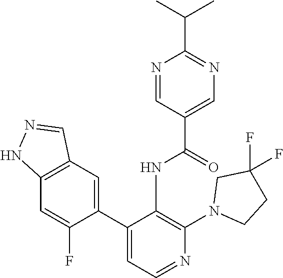 [SMILES: CC(C)c1ncc(C(=O)Nc2c(-c3cc4cn[nH]c4cc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0750 g, 0.158 mmol), 6-fluoro-4-[(4,4,5,5-tetramethyl-1,3,2-dioxaborolan-2-yl)methyl]-1H-indazole (50% pure, 0.175 g, 0.317 mmol), and potassium carbonate (0.0540 g, 0.396 mmol) in degassed 1,4-dioxane (1.50 mL) and water (0.450 mL) was added [1,1′-bis(diphenylphosphino)ferrocene]dichloropalladium(II), complex with dichloromethane (0.0380 g, 0.0470 mmol) and the mixture was stirred at 100° C. for 48 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by column chromatography, eluting with a gradient of 0-10% of methanol in dichloromethane, followed by preparative reverse phase HPLC, eluting with a gradient of 35-45% acetonitrile in water containing 10 mM of ammonium formate afforded the title compound as a solid (0.0190 g, 25% yield): ¹H NMR (500 MHz; DMSO-d₆) δ 13.15 (s, 1H), 10.18 (s, 1H), 8.84 (s, 2H), 8.22 (d, J=5.0 Hz, 1H), 8.09 (s, 1H), 7.70 (d, J=7.0 Hz, 1H), 7.40 (d, J=10.4 Hz, 1H), 6.85 (d, J=4.9 Hz, 1H), 3.90 (bs, 2H), 3.76 (bs, 2H), 3.13 (dt, J=13.8, 6.9 Hz, 1H), 2.49-2.38 (m, 2H), 1.24 (d, J=6.9 Hz, 6H); MS (ES+) m/z 482.2 (M+1).

### Example 41

### Synthesis of N-[2-(3,3-difluoropyrrolidin-1-yl)-4-(4-fluoro-1H-indazol-5-yl)-3-pyridyl]-2-isopropyl-pyrimidine-5-carboxamide

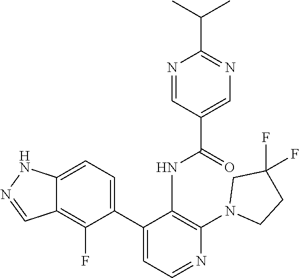 [SMILES: CC(C)c1ncc(C(=O)Nc2c(-c3ccc4[nH]ncc4c3F)ccnc2N2CCC(F)(F)C2)cn1]

### Step 1. Preparation of 4-fluoro-5-(4,4,5,5-tetramethyl-1,3,2-dioxaborolan-2-yl)-1H-indazole

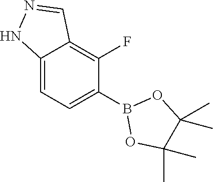 [SMILES: CC1(C)OB(c2ccc3[nH]ncc3c2F)OC1(C)C]

To a solution of 5-bromo-4-fluoro-1H-indazole (0.750 g, 3.49 mmol), 4,4,5,5-tetramethyl-2-(4,4,5,5-tetramethyl-1,3,2-dioxaborolan-2-yl)-1,3,2-dioxaborolane (1.94 g, 7.67 mmol) and [1,1′ bis(diphenylphosphino)ferrocene]dichloropalladium(II), complex with dichloromethane (0.570 g, 0.698 mmol) in 1,4-dioxane (6.00 mL) was added potassium acetate (1.02 g, 10.5 mmol), and the mixture was stirred at 100° C. for 20 h. After cooling to ambient temperature, the mixture was diluted with ethyl acetate (50 mL), and passed through a bed of diatomaceous earth (i.e., Celite®). The solid was washed with ethyl acetate (50 mL) and the filtrate was concentrated in vacuo. Purification of the residue by column chromatography, eluting with a gradient of 0-100% of methanol in dichloromethane, afforded the title compound as a brown oil (50% pure, 960 mg, 52% yield): ¹H NMR (300 MHz; DMSO-d₆) δ 13.39 (s, 1H), 7.93 (s, 1H), 7.53 (dd, J=8.3, 5.3 Hz, 1H), 6.88 (dd, J=10.6, 7.5 Hz, 1H), 1.07 (s, 12H); MS (ES+) m/z 263.1 (M+1).

### Step 2. Preparation of N-[2-(3,3-difluoropyrrolidin-1-yl)-4-(4-fluoro-1H-indazol-5-yl)-3-pyridyl]-2-isopropyl-pyrimidine-5-carboxamide

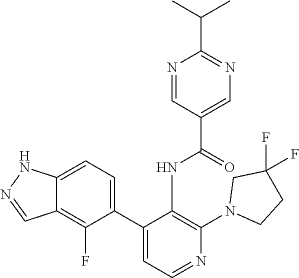 [SMILES: CC(C)c1ncc(C(=O)Nc2c(-c3ccc4[nH]ncc4c3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0750 g, 0.158 mmol), 4-fluoro-5-(4,4,5,5-tetramethyl-1,3,2-dioxaborolan-2-yl)-1H-indazole (50% pure, 0.166 g, 0.317 mmol), and potassium carbonate (0.0540 g, 0.396 mmol) in degassed 1,4-dioxane (1.50 mL) and water (0.450 mL) was added [1,1′-bis(diphenylphosphino)ferrocene]dichloropalladium(II), complex with dichloromethane (0.0380 g, 0.0470 mmol) and the mixture was stirred at 100° C. for 48 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by column chromatography, eluting with a gradient of 0-10% of methanol in dichloromethane, followed by preparative reverse phase HPLC, eluting with a gradient of 36-46% acetonitrile in water containing 10 mM of ammonium formate, afforded the title compound as a solid (0.0270 g, 35% yield): ¹H NMR (500 MHz; DMSO-d₆) δ 13.42 (s, 1H), 10.18 (s, 1H), 8.86 (s, 2H), 8.29-8.14 (m, 2H), 7.46-7.31 (m, 1H), 7.25 (d, J=7.1 Hz, 1H), 6.87 (d, J=4.9 Hz, 1H), 3.91 (s, 2H), 3.76 (s, 2H), 3.14 (dt, J=13.8, 6.9 Hz, 1H), 2.44 (dt, J=21.2, 7.0 Hz, 2H), 1.24 (d, J=6.9 Hz, 6H); MS (ES+) m/z 482.3 (M+1).

### Example 42

### Synthesis of N-[2-(3,3-difluoropyrrolidin-1-yl)-4-(1H-pyrazol-5-yl)-3-pyridyl]-2-isopropyl-pyrimidine-5-carboxamide

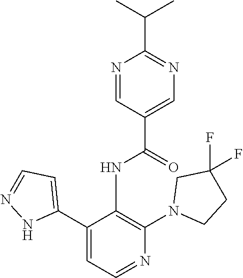 [SMILES: CC(C)c1ncc(C(=O)Nc2c(-c3ccn[nH]3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), 3-(4,4,5,5-tetramethyl-1,3,2-dioxaborolan-2-yl)-1H-pyrazole (0.0410 g, 0.201 mmol), and potassium carbonate (0.0350 g, 0.253 mmol) in 1,4-dioxane (1.20 mL) and water (0.400 mL) was added [1,1′-bis(diphenylphosphino)ferrocene]dichloropalladium(II), complex with dichloromethane (0.025 g, 0.0306 mmol), and the mixture was stirred at 100° C. for 2 h. After cooling to ambient temperature, the mixture was diluted with ethyl acetate (10 mL). The mixture was filtered through a bed of diatomaceous earth (i.e., Celite®). The solid was washed with ethyl acetate (50 mL), and the filtrate was concentrated in vacuo. Purification of the residue by column chromatography, eluting with a gradient of 0 to 15% of methanol in dichloromethane, followed by preparative reverse phase HPLC, eluting with a gradient of 50-60% acetonitrile in water containing 10 mM of ammonium formate, afforded the title compound as a colorless solid (0.0230 g, 55% yield): ¹H NMR (400 MHz; DMSO-d₆) δ 13.09 (bs, 1H), 10.23 (s, 1H), 9.17 (s, 2H), 8.09 (d, J=5.4 Hz, 1H), 7.91-7.51 (m, 1H), 7.14 (d, J=3.3 Hz, 1H), 6.81-6.43 (m, 1H), 4.12-3.50 (m, 4H), 3.23-3.13 (m, 1H), 2.36 (sept, J=7.9 Hz, 2H), 1.27 (d, J=6.9 Hz, 6H); MS (ES+) m/z 414.3 (M+1).

### Example 43

### Synthesis of N-[4-(cyclopenten-1-yl)-2-(3,3-difluoropyrrolidin-1-yl)-3-pyridyl]-2-isopropyl-pyrimidine-5-carboxamide

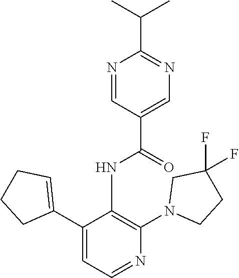 [SMILES: CC(C)c1ncc(C(=O)Nc2c(C3=CCCC3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.150 g, 0.301 mmol), 2-(cyclopenten-1-yl)-4,4,5,5-tetramethyl-1,3,2-dioxaborolane (0.123 g, 0.602 mmol), and potassium carbonate (0.104 g, 0.753 mmol) in 1,4-dioxane (3.00 mL) and water (0.900 mL) was added [1,1′-bis(diphenylphosphino)ferrocene]dichloropalladium(II), complex with dichloromethane (0.074 g, 0.0903 mmol), and the mixture was stirred at 80° C. for 2 h. After cooling to ambient temperature, the mixture was diluted with ethyl acetate (10 mL). The mixture was filtered through a bed of diatomaceous earth (i.e., Celite®). The solid was washed with ethyl acetate (40 mL), and the filtrate was concentrated in vacuo. Purification of the residue by column chromatography, eluting with a gradient of 0-5% of methanol in dichloromethane, afforded the title compound as a brown solid (0.129 g, 93% yield). Purification of the residue (0.0300 g) by preparative reverse phase HPLC, eluting with a gradient of 49-59% acetonitrile in water containing 10 mM of ammonium formate, afforded the title compound as a colorless solid (0.0200 g): ¹H NMR (400 MHz; DMSO-d₆) δ 510.13 (s, 1H), 9.19 (s, 2H), 8.06 (d, J=5.0 Hz, 1H), 6.78 (d, J=5.1 Hz, 1H), 6.08-5.96 (m, 1H), 3.92-3.63 (m, 4H), 3.23 (sept, J=6.9 Hz, 1H), 2.62-2.51 (m, 2H), 2.50-2.28 (m, 4H), 1.81 (p, J=7.5 Hz, 2H), 1.32 (d, J=6.9 Hz, 6H); MS (ES+) m/z 414.3 (M+1).

### Example 44

### Synthesis of N-[2-(3,3-difluoropyrrolidin-1-yl)-4-(5-fluoro-2-methoxy-phenyl)-3-pyridyl]-2-isopropyl-pyrimidine-5-carboxamide

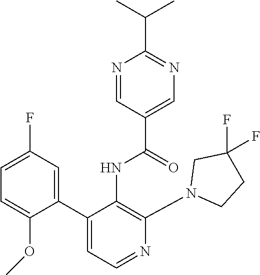 [SMILES: COc1ccc(F)cc1-c1ccnc(N2CCC(F)(F)C2)c1NC(=O)c1cnc(C(C)C)nc1]

To a solution of N-[2-(3,3-difluoropyrrolidin-1-yl)-4-iodo-3-pyridyl]-2-isopropyl-pyrimidine-5-carboxamide (0.0500 g, 0.100 mmol), (5-fluoro-2-methoxy-phenyl)boronic acid (0.0360 g, 0.201 mmol), and potassium carbonate (0.0350 g, 0.253 mmol) in 1,4-dioxane (1.200 mL) and water (0.400 mL) was added [1,1′-bis(diphenylphosphino)ferrocene]dichloropalladium(II), complex with dichloromethane (0.0250 g, 0.0306 mmol), and the mixture was stirred at 90° C. for 1 h. After cooling to ambient temperature, the mixture was diluted with ethyl acetate (10 mL). The mixture was filtered through a bed of diatomaceous earth (i.e., Celite®). The solid was washed with ethyl acetate (50 mL), and the filtrate concentrated in vacuo. Purification of the residue by column chromatography, eluting with a gradient of 0-10% of methanol in dichloromethane, followed by preparative reverse phase HPLC, eluting with a gradient of 45-55% acetonitrile in water containing 10 mM of ammonium formate, afforded the title compound as a colorless solid (0.032 g, 62% yield): ¹H NMR (400 MHz; DMSO-d₆) δ 9.94 (s, 1H), 8.84 (s, 2H), 8.16 (d, J=4.9 Hz, 1H), 7.18-7.08 (m, 1H), 7.04 (dd, J=9.2, 4.6 Hz, 1H), 7.00-6.87 (m, 1H), 6.73 (d, J=4.9 Hz, 1H), 4.01-3.71 (m, 4H), 3.69 (s, 3H), 3.22-3.12 (m, 1H), 2.42 (dt, J=21.8, 7.1 Hz, 2H), 1.27 (d, J=6.9 Hz, 6H); MS (ES+) m/z 472.3 (M+1).

### Example 45

### Synthesis of N-[2-(3,3-difluoropyrrolidin-1-yl)-4-(5-fluoro-2-methyl-phenyl)-3-pyridyl]-2-isopropyl-pyrimidine-5-carboxamide

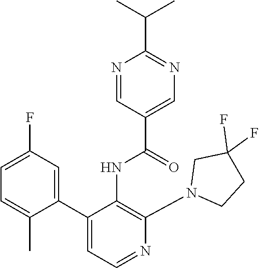 [SMILES: Cc1ccc(F)cc1-c1ccnc(N2CCC(F)(F)C2)c1NC(=O)c1cnc(C(C)C)nc1]

To a solution of N-[2-(3,3-difluoropyrrolidin-1-yl)-4-iodo-3-pyridyl]-2-isopropyl-pyrimidine-5-carboxamide (0.0500 g, 0.100 mmol), (5-fluoro-2-methyl-phenyl)boronic acid (0.0330 g, 0.201 mmol), and potassium carbonate (0.0350 g, 0.253 mmol) in 1,4-dioxane (1.20 mL) and water (0.400 mL) was added [1,1′-bis(diphenylphosphino)ferrocene]dichloropalladium(II), complex with dichloromethane (0.0250 g, 0.0301 mmol), and the mixture was stirred at 90° C. for 1 h. After cooling to ambient temperature, the mixture was diluted with ethyl acetate (10 mL). The mixture was filtered through a bed of diatomaceous earth (i.e., Celite®). The solid was washed with ethyl acetate (50 mL), and the filtrate concentrated in vacuo. Purification of the residue by column chromatography, eluting with a gradient of 0-10% of methanol in dichloromethane, followed by preparative reverse phase HPLC, eluting with a gradient of 45-55% acetonitrile in water containing 10 mM of ammonium formate, afforded the title compound as a colorless solid (0.0320 g, 62% yield): ¹H NMR (400 MHz; DMSO-d₆) δ 10.01 (s, 1H), 8.77 (s, 2H), 8.19 (d, J=5.0 Hz, 1H), 7.33-7.14 (m, 1H), 7.03 (dd, J=8.6, 5.8 Hz, 1H), 6.98-6.80 (m, 1H), 6.72 (d, J=5.2 Hz, 1H), 4.03-3.62 (m, 4H), 3.16 (dt, J=13.8, 6.8 Hz, 1H), 2.49-2.37 (m, 2H), 2.08 (s, 3H), 1.25 (d, J=6.9 Hz, 6H); MS (ES+) m/z 456.3 (M+1).

### Example 46

### Synthesis of N-[4-cyclopentyl-2-(3,3-difluoropyrrolidin-1-yl)-3-pyridyl]-2-isopropyl-pyrimidine-5-carboxamide

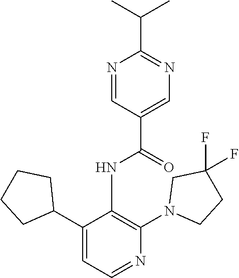 [SMILES: CC(C)c1ncc(C(=O)Nc2c(C3CCCC3)ccnc2N2CCC(F)(F)C2)cn1]

To a solution of palladium (10% on carbon matrix, 0.0610 g, 0.0570 mmol) in methanol (1.00 mL) was added a solution of N-[4-(cyclopenten-1-yl)-2-(3,3-difluoropyrrolidin-1-yl)-3-pyridyl]-2-isopropyl-pyrimidine-5-carboxamide (0.0500 g, 0.120 mmol) in methanol (1.00 mL). The mixture was stirred at 22° C. for 1 h under hydrogen. The mixture was diluted with dichloromethane (10 mL), and filtered through a bed of diatomaceous earth (i.e., Celite®). The solid was washed with dichloromethane (50 mL), and the filtrate concentrated in vacuo. Purification of the residue by preparative reverse phase HPLC, eluting with a gradient of 49-59% acetonitrile in water containing 10 mM of ammonium formate, afforded the title compound as a solid (0.0250 g, 52% yield): ¹H NMR (400 MHz; DMSO-d₆) δ 10.14 (s, 1H), 9.25 (s, 2H), 8.06 (d, J=5.1 Hz, 1H), 6.81 (d, J=5.3 Hz, 1H), 3.95-3.57 (m, 4H), 3.23 (dq, J=13.8, 6.9 Hz, 1H), 3.10-3.02 (m, 1H), 2.39 (tt, J=14.0, 7.1 Hz, 2H), 1.96-1.83 (m, 2H), 1.78-1.62 (m, 2H), 1.60-1.43 (m, 4H), 1.32 (d, J=6.9 Hz, 6H); MS (ES+) m/z 416.3 (M+1).

### Example 47

### Synthesis of N-[2-(3,3-difluoropyrrolidin-1-yl)-4-(1-methylpyrazol-3-yl)-3-pyridyl]-2-isopropyl-pyrimidine-5-carboxamide

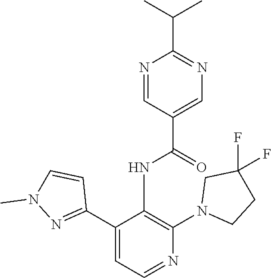 [SMILES: CC(C)c1ncc(C(=O)Nc2c(-c3ccn(C)n3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), (1-methylpyrazol-3-yl)boronic acid (0.0266 g, 0.201 mmol), and potassium carbonate (0.0347 g, 0.251 mmol) in 1,4-dioxane (1.20 mL) and water (0.400 mL) was added [1,1′-bis(diphenylphosphino)ferrocene]dichloropalladium(II), complex with dichloromethane (0.0246 g, 0.0306 mmol), and the mixture was stirred at 90° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate was concentrated in vacuo. Purification of the residue by column chromatography, eluting with a gradient of 0-10% of methanol in dichloromethane, followed by preparative reverse phase HPLC, eluting with a gradient of 33-43% acetonitrile in water containing 10 mM of ammonium formate, afforded the title compound as a colorless solid (0.0336 g, 78% yield): ¹H NMR (400 MHz; DMSO-d₆) δ 10.24 (s, 1H), 9.22 (s, 2H), 8.13 (d, J=5.1 Hz, 1H), 7.70 (d, J=2.2 Hz, 1H), 7.16 (d, J=5.2 Hz, 1H), 6.60 (d, J=2.3 Hz, 1H), 3.96-3.66 (m, 4H), 3.78 (s, 3H), 3.28-3.18 (m, 1H), 2.47-2.35 (m, 2H), 1.32 (d, J=6.9 Hz, 6H); MS (ES+) m/z 428.3 (M+1).

### Example 48

### Synthesis of N-[2-(3,3-difluoropyrrolidin-1-yl)-4-(4-fluoro-2-methyl-pyrazol-3-yl)-3-pyridyl]-2-isopropyl-pyrimidine-5-carboxamide

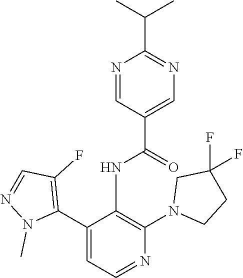 [SMILES: CC(C)c1ncc(C(=O)Nc2c(-c3c(F)cnn3C)ccnc2N2CCC(F)(F)C2)cn1]

### Step 1. Preparation of 4-fluoro-1-methyl-5-(4,4,5,5-tetramethyl-1,3,2-dioxaborolan-2-yl)pyrazole

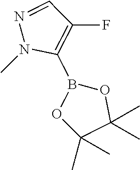

To a solution of 5-bromo-4-fluoro-1-methyl-pyrazole (0.330 g, 1.84 mmol), 4,4,5,5-tetramethyl-2-(4,4,5,5-tetramethyl-1,3,2-dioxaborolan-2-yl)-1,3,2-dioxaborolane (1.03 g, 4.06 mmol) and [1,1′-bis(diphenylphosphino)ferrocene]dichloropalladium(II), complex with dichloromethane (0.301 g, 0.369 mmol) in 1,4-dioxane (4.76 mL) was added potassium acetate (0.543 g, 5.53 mmol), and the mixture was stirred at 100° C. for 20 h. After cooling to ambient temperature, the mixture was diluted with EtOAc (20 mL). The mixture was filtered through a bed of diatomaceous earth (i.e., Celite®). The solid was washed with EtOAc (20 mL) and the filtrate was concentrated in vacuo.

Purification of the residue by column chromatography, eluting with a gradient of 0-10% of methanol in dichloromethane, afforded the title compound as a colorless solid (60% pure, 261 mg, 34%): ¹H NMR (300 MHz; CDCl₃) δ 7.28 (d, J=4.4 Hz, 1H), 3.98 (s, 3H), 1.35 (s, 12H); ¹⁹F NMR (376 MHz; CDCl₃) 5-166.15 (d, J=4.4 Hz).

### Step 2. Preparation of N-[2-(3,3-difluoropyrrolidin-1-yl)-4-(4-fluoro-2-methyl-pyrazol-3-yl)-3 pyridyl]-2-isopropyl-pyrimidine-5-carboxamide

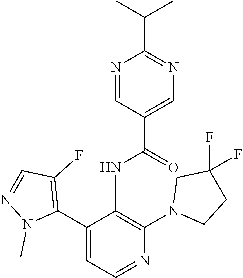 [SMILES: CC(C)c1ncc(C(=O)Nc2c(-c3c(F)cnn3C)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), 4-fluoro-1-methyl-5-(4,4,5,5-tetramethyl-1,3,2-dioxaborolan-2-yl)pyrazole (60% pure, 0.0756 g, 0.201 mmol), and potassium carbonate (0.0347 g, 0.251 mmol) in 1,4-dioxane (1.20 mL) and water (0.400 mL) was added [1,1′-bis(diphenylphosphino)ferrocene]dichloropalladium(II), complex with dichloromethane (0.0246 g, 0.0306 mmol), and the mixture was stirred at 90° C. for 3 h. 4-fluoro-1-methyl-5-(4,4,5,5-tetramethyl-1,3,2-dioxaborolan-2-yl)pyrazole (60% pure, 0.184 g, 0.489 mmol), potassium carbonate (0.0694 g, 0.502 mmol) and [1,1′-bis(diphenylphosphino)ferrocene]dichloropalladium(II), complex with dichloromethane (0.0246 g, 0.0306 mmol) was added and the mixture was stirred at 100° C. for 72 h. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate was concentrated in vacuo. Purification of the residue by column chromatography, eluting with a gradient of 0-10% of methanol in dichloromethane, followed by preparative reverse phase HPLC, eluting with a gradient of 37-47% acetonitrile in water containing 10 mM of ammonium formate, afforded the title compound as a white solid (0.007 g, 16% yield): ¹H NMR (400 MHz; DMSO-d₆) δ 10.36 (s, 1H), 8.99 (s, 2H), 8.28 (d, J=4.9 Hz, 1H), 7.44 (d, J=4.3 Hz, 1H), 6.95 (d, J=4.9 Hz, 1H), 3.98-3.69 (m, 4H), 3.65 (s, 3H), 3.19 (dt, J=13.9, 6.9 Hz, 1H), 2.43 (td, J=13.9, 6.8 Hz, 2H), 1.29 (d, J=6.9 Hz, 6H); MS (ES+) m/z 446.3 (M+1).

### Example 49

### Synthesis of N-[4-(5-cyano-2-fluoro-phenyl)-2-(3,3-difluoropyrrolidin-1-yl)-3-pyridyl]-2-isopropyl-pyrimidine-5-carboxamide

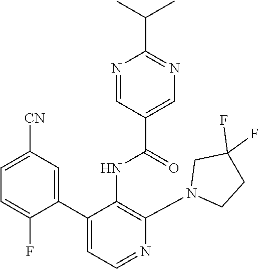 [SMILES: CC(C)c1ncc(C(=O)Nc2c(-c3cc(C#N)ccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.106 mmol), (5-cyano-2-fluoro-phenyl)boronic acid (0.0261 g, 0.158 mmol) and [1,1′ bis(diphenylphosphino)ferrocene]dichloropalladium(II), complex with dichloromethane (0.0173 g, 0.0211 mmol) in 1,4-dioxane (1.00 mL) and water (0.250 mL) was added potassium carbonate (0.0365 g, 0.264 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate, followed by preparative reverse phase HPLC, eluting with a gradient of 45-55% of acetonitrile in water containing 10 mM of ammonium formate, afforded the title compound as a colorless solid (0.0240 g, 48% yield): ¹H NMR (400 MHz; DMSO-d₆) δ 10.25 (s, 1H), 8.90 (s, 2H), 8.25 (d, J=5.0 Hz, 1H), 7.96-7.87 (m, 1H), 7.83 (d, J=4.7 Hz, 1H), 7.58-7.48 (m, 1H), 6.87 (d, J=4.9 Hz, 1H), 4.00-3.83 (m, 2H), 3.76 (tt, J=14.0, 7.1 Hz, 2H), 3.18 (dt, J=13.8, 6.9 Hz, 1H), 2.44 (dd, J=14.2, 7.0 Hz, 2H), 1.27 (d, J=6.9 Hz, 6H); MS (ES+) m/z 467.3 (M+1).

### Example 50

### Synthesis of N-[2-(3,3-difluoropyrrolidin-1-yl)-4-(2-fluoro-5-methoxy-phenyl)-3-pyridyl]-2-isopropyl-pyrimidine-5-carboxamide

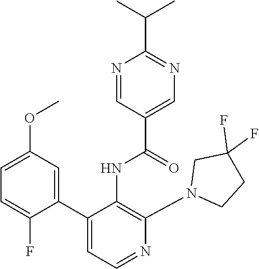 [SMILES: COc1ccc(F)c(-c2ccnc(N3CCC(F)(F)C3)c2NC(=O)c2cnc(C(C)C)nc2)c1]

To a solution of N-[2-(3,3-difluoropyrrolidin-1-yl)-4-iodo-3-pyridyl]-2-isopropyl-pyrimidine-5-carboxamide (0.0500 g, 0.106 mmol), (2-fluoro-5-methoxy-phenyl)boronic acid (0.0269 g, 0.158 mmol) and [1,1′ bis(diphenylphosphino)ferrocene]dichloropalladium(II), complex with dichloromethane (0.0173 g, 0.0211 mmol) in 1,4-dioxane (1.00 mL) and water (0.250 mL) was added potassium carbonate (0.0365 g, 0.264 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate followed by preparative reverse phase HPLC, eluting with a gradient of 43-53% of acetonitrile in water containing 10 mM of ammonium formate, afforded the title compound as a colorless solid (0.0260 g, 52% yield): ¹H NMR (400 MHz; DMSO-d₆) δ 10.16 (s, 1H), 8.90 (s, 2H), 8.21 (d, J=5.0 Hz, 1H), 7.17 (t, J=9.2 Hz, 1H), 6.90 (dt, J=9.0, 3.8 Hz, 1H), 6.87-6.75 (m, 2H), 4.00-3.82 (m, 2H), 3.77 (ddd, J=23.6, 16.6, 7.1 Hz, 2H), 3.65 (s, 3H), 3.25-3.11 (m, 1H), 2.43 (dt, J=21.3, 7.2 Hz, 2H), 1.27 (d, J=6.9 Hz, 6H); MS (ES+) m/z 472.2 (M+1).

### Example 51

### Synthesis of N-[4-(5-chloro-2-fluoro-phenyl)-2-(3,3-difluoropyrrolidin-1-yl)-3-pyridyl]-2-isopropyl-pyrimidine-5-carboxamide

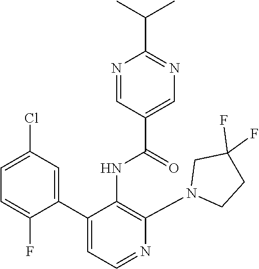 [SMILES: CC(C)c1ncc(C(=O)Nc2c(-c3cc(Cl)ccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.106 mmol), (5-chloro-2-fluoro-phenyl)boronic acid (0.0184 g, 0.106 mmol) and [1,1′ bis(diphenylphosphino)ferrocene]dichloropalladium(II), complex with dichloromethane (0.0173 g, 0.0211 mmol) in 1,4-dioxane (1.00 mL) and water (0.250 mL) was added potassium carbonate (0.0365 g, 0.264 mmol), and the mixture was stirred at 100° C. for 2 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate, followed by preparative reverse phase HPLC, eluting with a gradient of 28-38% of acetonitrile in water containing 10 mM of ammonium formate, afforded the title compound as a colorless solid (0.00800 g, 16% yield): ¹H NMR (400 MHz; DMSO-d₆) δ 10.24 (s, 1H), 8.92 (s, 2H), 8.23 (d, J=5.0 Hz, 1H), 7.39 (ddd, J=28.1, 13.6, 7.6 Hz, 3H), 6.86 (d, J=4.9 Hz, 1H), 4.01-3.84 (m, 2H), 3.84-3.63 (m, 2H), 3.19 (dt, J=13.8,6.8 Hz, 1H), 2.44 (dd, J=14.1, 6.9 Hz, 2H), 1.28 (d, J=6.9 Hz, 6H); MS (ES+) m/z 476.2 (M+1).

### Example 52

### Synthesis of N-[2-(3,3-difluoropyrrolidin-1-yl)-4-[2-fluoro-5-(methylcarbamoyl)phenyl]-3-pyridyl]-2-isopropyl-pyrimidine-5-carboxamide

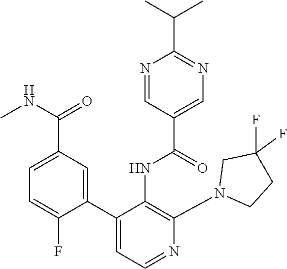 [SMILES: CNC(=O)c1ccc(F)c(-c2ccnc(N3CCC(F)(F)C3)c2NC(=O)c2cnc(C(C)C)nc2)c1]

To a solution of N-[2-(3,3-difluoropyrrolidin-1-yl)-4-iodo-3-pyridyl]-2-isopropyl-pyrimidine-5-carboxamide (0.0500 g, 0.100 mmol), [2-fluoro-5-(methylcarbamoyl)phenyl]boronic acid (0.0416 g, 0.201 mmol) and potassium carbonate (0.0347 g, 0.251 mmol) in 1,4-dioxane (1.20 mL) and water (0.40 mL) was added [1,1′-bis(diphenylphosphino)ferrocene]dichloropalladium(II), complex with dichloromethane (0.0246 g, 0.0301 mmol), and the mixture was stirred at 100° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate was concentrated in vacuo. Purification of the residue by column chromatography, eluting with a gradient of 0-15% of methanol in dichloromethane, followed by preparative reverse phase HPLC, eluting with a gradient of 36-46% acetonitrile in water containing 10 mM of ammonium formate, afforded the title compound as a colorless solid (0.024 g, 48% yield): ¹H NMR (400 MHz; DMSO-d₆) δ 10.24 (s, 1H), 8.86 (s, 2H), 8.44 (d, J=4.1 Hz, 1H), 8.23 (d, J=4.9 Hz, 1H), 7.87-7.74 (m, 2H), 7.34 (t, J=9.2 Hz, 1H), 6.85 (d, J=4.9 Hz, 1H), 4.01-3.61 (m, 4H), 3.15 (sept, J=6.9 Hz, 1H), 2.74 (d, J=4.5 Hz, 3H), 2.48-2.38 (m, 2H), 1.26 (d, J=6.9 Hz, 6H); MS (ES+) m/z 499.3 (M+1).

### Example 53

### Synthesis of N-[2-(3,3-difluoropyrrolidin-1-yl)-4-[5-(dimethylcarbamoyl)-2-fluoro-phenyl]-3-pyridyl]-2-isopropyl-pyrimidine-5-carboxamide

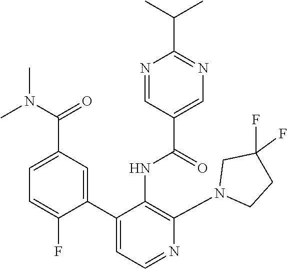 [SMILES: CC(C)c1ncc(C(=O)Nc2c(-c3cc(C(=O)N(C)C)ccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), [5-(dimethylcarbamoyl)-2-fluoro-phenyl]boronic acid (0.0446 g, 0.201 mmol), and potassium carbonate (0.0347 g, 0.251 mmol) in 1,4-dioxane (1.20 mL) and water (0.400 mL) was added [1,1′-bis(diphenylphosphino)ferrocene]dichloropalladium(II), complex with dichloromethane (0.0246 g, 0.0301 mmol), and the mixture was stirred at 100° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL), and the filtrate was concentrated in vacuo. Purification of the residue by column chromatography, eluting with a gradient of 0-10% of methanol in dichloromethane, followed by preparative reverse phase HPLC, eluting with a gradient of 34-44% acetonitrile in water containing 10 mM of ammonium formate, afforded the title compound as a white solid (0.042 g, 81% yield): ¹H NMR (400 MHz; DMSO-d₆) δ 10.22 (s, 1H), 8.90 (s, 2H), 8.22 (d, J=5.0 Hz, 1H), 7.43-7.35 (m, 1H), 7.31 (dd, J=11.9, 6.0 Hz, 2H), 6.85 (d, J=5.1 Hz, 1H), 4.03-3.70 (m, 4H), 3.24-3.11 (m, 1H), 2.91 (bs, 3H), 2.64 (bs, 3H), 2.49-2.34 (m, 2H), 1.26 (d, J=6.9 Hz, 6H); MS (ES+) m/z 513.3 (M+1).

### Example 54

### Synthesis of N-[2-(3,3-difluoropyrrolidin-1-yl)-4-[2-fluoro-5-(hydroxymethyl)phenyl]-3-pyridyl]-2-isopropyl-pyrimidine-5-carboxamide

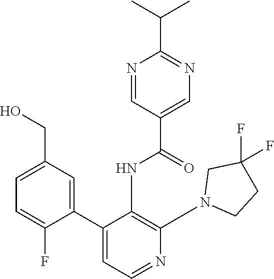 [SMILES: CC(C)c1ncc(C(=O)Nc2c(-c3cc(CO)ccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol), [2-fluoro-5-(hydroxymethyl)phenyl]boronic acid (0.035.9 g, 0.201 mmol), and potassium carbonate (0.0347 g, 0.251 mmol) in 1,4-dioxane (1.20 mL) and water (0.400 mL) was added [1,1′-bis(diphenylphosphino)ferrocene]dichloropalladium(II), complex with dichloromethane (0.0246 g, 0.0306 mmol), and the mixture was stirred at 100° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL) and the filtrate was concentrated in vacuo. Purification of the residue by column chromatography, eluting with a gradient of 0-10% of methanol in dichloromethane, followed by preparative reverse phase HPLC, eluting with a gradient of 32-42% acetonitrile in water containing 10 mM of ammonium formate, afforded the title compound as a colorless solid (0.0368 g, 78% yield): ¹H NMR (400 MHz; DMSO-d₆) δ 10.16 (s, 1H), 8.88 (s, 2H), 8.20 (d, J=5.0 Hz, 1H), 7.32-7.23 (m, 2H), 7.23-7.14 (m, 1H), 6.79 (d, J=4.8 Hz, 1H), 5.23 (t, J=5.5 Hz, 1H), 4.40 (d, J=5.5 Hz, 2H), 4.07-3.56 (m, 4H), 3.23-3.09 (m, 1H), 2.48-2.33 (m, 2H), 1.27 (d, J=6.9 Hz, 6H); MS (ES+) m/z 472.3 (M+1).

### Example 55

### Synthesis of N-[2-(3,3-difluoropyrrolidin-1-yl)-4-[2-fluoro-5-(morpholinomethyl)phenyl]-3-pyridyl]-2-isopropyl-pyrimidine-5-carboxamide

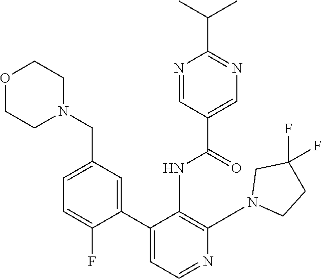 [SMILES: CC(C)c1ncc(C(=O)Nc2c(-c3cc(CN4CCOCC4)ccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.100 mmol),4-[[4-fluoro-3-(4,4,5,5-tetramethyl-1,3,2-dioxaborolan-2-yl)phenyl]methyl]morpholine (0.0679 g, 0.201 mmol), and potassium carbonate (0.0347 g, 0.251 mmol) in 1,4-dioxane (1.20 mL) and water (0.400 mL) was added [1,1′-bis(diphenylphosphino)ferrocene]dichloropalladium(II), complex with dichloromethane (0.0246 g, 0.0306 mmol), and the mixture was stirred at 100° C. for 1 h. After cooling to ambient temperature, the mixture was diluted with ethyl acetate (10 mL). The mixture was filtered through a bed of diatomaceous earth (i.e., Celite®). The solid was washed with ethyl acetate (30 mL) and the filtrate was concentrated in vacuo. Purification of the residue by column chromatography, eluting with a gradient of 0-10% of methanol in dichloromethane, followed by preparative reverse phase HPLC, eluting with a gradient of 43-53% acetonitrile in water containing 10 mM of ammonium bicarbonate, by reverse phase chromatography, eluting with a gradient of 5-100% acetonitrile in water containing 10 mM of ammonium formate, and finally again by preparative reverse phase HPLC, eluting with a gradient of 43-53% acetonitrile in water containing 10 mM of ammonium bicarbonate, afforded the title compound as a colorless solid (0.012 g, 23% yield): ¹H NMR (400 MHz; DMSO-d₆) δ 10.19 (s, 1H), 8.91 (s, 2H), 8.20 (d, J=5.0 Hz, 1H), 7.45-7.06 (m, 3H), 6.80 (dd, J=5.0, 0.9 Hz, 1H), 4.11-3.51 (m, 4H), 3.44-3.34 (m, 4H), 3.32 (s, 2H), 3.23-3.07 (m, 1H), 2.48-2.37 (m, 2H), 2.19-2.07 (m, 4H), 1.26 (d, J=6.9 Hz, 6H); MS (ES+) m/z 541.3 (M+1).

### Example 56

### Synthesis of N-[2-(3,3-difluoropyrrolidin-1-yl)-4-(2-fluoro-5-methoxy-phenyl)-3-pyridyl]-6-isopropyl-pyridine-3-carboxamide

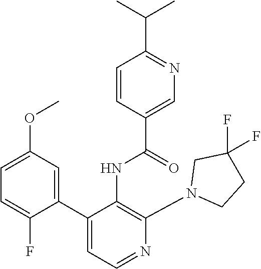 [SMILES: COc1ccc(F)c(-c2ccnc(N3CCC(F)(F)C3)c2NC(=O)c2ccc(C(C)C)nc2)c1]

### Step 1. Preparation of 2-(3,3-difluoropyrrolidin-1-yl)-4-(2-fluoro-5-methoxy-phenyl)pyridin-3-amine

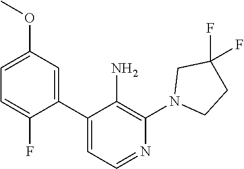 [SMILES: COc1ccc(F)c(-c2ccnc(N3CCC(F)(F)C3)c2N)c1]

To a solution of 2-(3,3-difluoropyrrolidin-1-yl)-4-iodo-pyridin-3-amine hydrochloride (0.800 g, 2.10 mmol), (2-fluoro-5-methoxy-phenyl)boronic acid (0.752 g, 4.20 mmol), and potassium carbonate (1.02 g, 7.36 mmol) in dioxane (25.1 mL) and water (8.38 mL) was added [1,1′-bis(diphenylphosphino)ferrocene]dichloropalladium(II), complex with dichloromethane (0.515 g, 0.631 mmol), and the mixture was stirred at 100° C. for 2 h. After cooling to ambient temperature, the mixture was diluted with saturated aqueous sodium bicarbonate solution (150 mL). The aqueous phase was extracted with ethyl acetate (3×100 mL). The organic phase was washed with brine (200 mL), dried over anhydrous sodium sulfate and concentrated in vacuo. Purifcation of the residue by column chromatography, eluting with a gradient of 0-40% of ethyl acetate in hexanes, afforded the title compound as a red oil (0.622 g, 82% yield): ¹H NMR (400 MHz; CDCl₃) δ 7.82 (d, J=5.0 Hz, 1H), 7.12 (t, J=9.1 Hz, 1H), 6.92 (ddd, J=9.0, 3.9, 3.2 Hz, 1H), 6.86 (dd, J=5.7, 3.2 Hz, 1H), 6.83 (dd, J=5.0, 0.7 Hz, 1H), 3.81 (s, 3H), 3.78 (s, 2H), 3.70 (t, J=13.1 Hz, 2H), 3.55 (t, J=7.1 Hz, 2H), 2.44 (hept, J=7.1 Hz, 2H); MS (ES+) m/z 324.5 (M+1).

### Step 2. Preparation of N-[2-(3,3-difluoropyrrolidin-1-yl)-4-(2-fluoro-5-methoxy-phenyl)-3-pyridyl]-6-isopropyl-pyridine-3-carboxamide

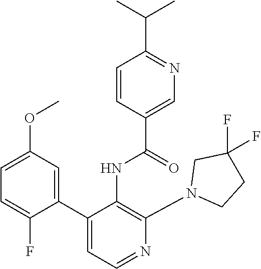 [SMILES: COc1ccc(F)c(-c2ccnc(N3CCC(F)(F)C3)c2NC(=O)c2ccc(C(C)C)nc2)c1]

To a solution of 2-(3,3-difluoropyrrolidin-1-yl)-4-(2-fluoro-5-methoxy-phenyl)pyridin-3-amine (0.0400 g, 0.111 mmol), 6-isopropylpyridine-3-carboxylic acid hydrochloride (0.0337 g, 0.167 mmol) and N,N-diisopropylethylamine (0.0762 mL, 0.445 mmol) in tetrahydrofuran (1.00 mL) was added 2-chloro-1-methyl-pyridin-1-ium iodide (0.114 g, 0.445 mmol), and the mixture was stirred at 65° C. for 20 h. After cooling to ambient temperature, the mixture was diluted with saturated aqueous sodium bicarbonate (15 mL), and the aqueous phase was extracted with ethyl acetate (3×15 mL). The organic phase was dried over anhydrous sodium sulfate, filtered, and concentrated in vacuo. Purification of the residue by column chromatography, eluting with a gradient of 0-15% of methanol in dichloromethane, followed by reverse phase chromatography, eluting with a gradient of 5-100% of acetonitrile in water containing 10 mM of ammonium bicarbonate, afforded the title compound as a colorless solid (0.045 g, 86% yield): ¹H NMR (400 MHz; DMSO-d₆) δ 9.91 (s, 1H), 8.69 (d, J=1.7 Hz, 1H), 8.14 (d, J=5.0 Hz, 1H), 7.90 (dd, J=8.2, 2.4 Hz, 1H), 7.32 (d, J=7.8 Hz, 1H), 7.11 (t, J=9.2 Hz, 1H), 6.89-6.81 (m, 1H), 6.78 (dd, J=5.9, 3.2 Hz, 1H), 6.75 (d, J=5.0 Hz, 1H), 4.03-3.62 (m, 4H), 3.59 (s, 3H), 3.00 (hept, J=7.0 Hz, 1H), 2.38 (dt, J=21.3, 7.2 Hz, 2H), 1.18 (d, J=6.9 Hz, 6H); MS (ES+) m/z 471.3 (M+1).

### Example 57

### Synthesis of N-[2-(3,3-difluoropyrrolidin-1-yl)-4-(3-methoxyphenyl)-3-pyridyl]-2-isopropyl-pyrimidine-5-carboxamide

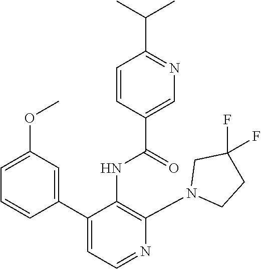 [SMILES: COc1cccc(-c2ccnc(N3CCC(F)(F)C3)c2NC(=O)c2ccc(C(C)C)nc2)c1]

To a solution of N-[2-(3,3-difluoropyrrolidin-1-yl)-4-iodo-3-pyridyl]-2-isopropyl-pyrimidine-5-carboxamide (0.0750 g, 0.158 mmol), (3-methoxyphenyl)boronic acid (0.0482 g, 0.317 mmol) and [1,1′ bis(diphenylphosphino)ferrocene]dichloropalladium(II), complex with dichloromethane (0.0259 g, 0.0317 mmol) in 1,4-dioxane (1.50 mL) and water (0.350 mL) was added potassium carbonate (0.0548 g, 0.396 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate, followed by preparative reverse phase HPLC, eluting with a gradient of 48-58% of acetonitrile in water containing 10 mM of ammonium formate, afforded the title compound as a colorless solid (0.0395 g, 50% yield): ¹H NMR (500 MHz; DMSO-d₆) δ 10.14 (s, 1H), 8.94 (s, 2H), 8.20 (d, J=5.0 Hz, 1H), 7.30 (t, J=7.9 Hz, 1H), 7.01-6.85 (m, 3H), 6.81 (d, J=5.0 Hz, 1H), 4.00-3.74 (m, 4H), 3.70 (s, 3H), 3.19 (dt, J=13.8, 6.9 Hz, 1H), 2.48-2.36 (m, 2H), 1.28 (d, J=6.9 Hz, 6H); MS (ES+) m/z 454.3 (M+1).

### Example 58

### Synthesis of N-[2-(3,3-difluoropyrrolidin-1-yl)-4-(5-ethoxy-2-fluoro-phenyl)-3-pyridyl]-2-isopropyl-pyrimidine-5-carboxamide

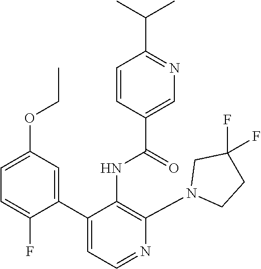 [SMILES: CCOc1ccc(F)c(-c2ccnc(N3CCC(F)(F)C3)c2NC(=O)c2ccc(C(C)C)nc2)c1]

To a solution of N-[2-(3,3-difluoropyrrolidin-1-yl)-4-iodo-3-pyridyl]-2-isopropyl-pyrimidine-5-carboxamide (0.0750 g, 0.158 mmol), (5-ethoxy-2-fluoro-phenyl)boronic acid (0.0583 g, 0.317 mmol) and [1,1′ bis(diphenylphosphino)ferrocene]dichloropalladium(II), complex with dichloromethane (0.0259 g, 0.0317 mmol) in 1,4-dioxane (1.50 mL) and water (0.350 mL) was added potassium carbonate (0.0548 g, 0.396 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate followed by preparative reverse phase HPLC, eluting with a gradient of 47-57% of acetonitrile in water containing 10 mM of ammonium formate, afforded the title compound as a colorless solid (0.0105 g, 13% yield): ¹H NMR (400 MHz; DMSO-d₆) δ 10.19 (s, 1H), 8.90 (s, 2H), 8.19 (d, J=4.9 Hz, 1H), 7.15 (t, J=9.2 Hz, 1H), 6.88 (dt, J=9.0, 3.6 Hz, 1H), 6.81 (d, J=4.7 Hz, 2H), 3.98-3.80 (m, 4H), 3.76 (s, 2H), 3.17 (dt, J=13.8, 6.9 Hz, 1H), 2.42 (dt, J=21.4, 7.1 Hz, 2H), 1.26 (d, J=6.9 Hz, 6H), 1.20 (t, J=7.0 Hz, 3H); MS (ES+) m/z 486.3 (M+1).

### Example 59

### Synthesis of N-[2-(3,3-difluoropyrrolidin-1-yl)-4-(2-fluoro-4-methoxy-phenyl)-3-pyridyl]-2-isopropyl-pyrimidine-5-carboxamide

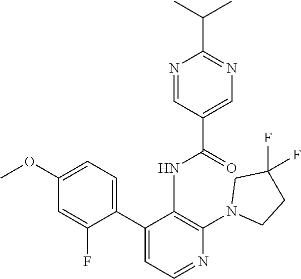 [SMILES: COc1ccc(-c2ccnc(N3CCC(F)(F)C3)c2NC(=O)c2cnc(C(C)C)nc2)c(F)c1]

To a solution of N-[2-(3,3-difluoropyrrolidin-1-yl)-4-iodo-3-pyridyl]-2-isopropyl-pyrimidine-5-carboxamide (0.0750 g, 0.158 mmol), (2-fluoro-4-methoxy-phenyl)boronic acid (0.0539 g, 0.317 mmol) and [1,1′ bis(diphenylphosphino)ferrocene]dichloropalladium(II), complex with dichloromethane (0.0259 g, 0.0317 mmol) in 1,4-dioxane (1.50 mL) and water (0.350 mL) was added potassium carbonate (0.0548 g, 0.396 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate, followed by preparative reverse phase HPLC, eluting with a gradient of 42-52% of acetonitrile in water containing 10 mM of ammonium formate, afforded the title compound as a colorless solid (0.0351 g, 47% yield): ¹H NMR (400 MHz; DMSO-d₆) δ 10.16 (s, 1H), 8.93 (s, 2H), 8.16 (d, J=4.2 Hz, 1H), 7.23 (t, J=8.7 Hz, 1H), 6.87 (dd, J=12.2, 2.3 Hz, 1H), 6.77 (dd, J=6.9, 3.7 Hz, 2H), 3.88 (bs, 2H), 3.72 (bs, 5H), 3.17 (dt, J=13.8, 6.9 Hz, 1H), 2.48-2.35 (m, 2H), 1.27 (d, J=6.9 Hz, 6H); MS (ES+) m/z 472.2 (M+1).

### Example 60

### Synthesis of N-[2-(3,3-difluoropyrrolidin-1-yl)-4-(2-fluoro-5-methoxy-phenyl)-3-pyridyl]-6-methoxy-pyridine-3-carboxamide

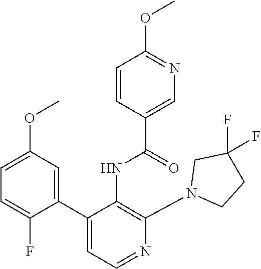 [SMILES: COc1ccc(F)c(-c2ccnc(N3CCC(F)(F)C3)c2NC(=O)c2ccc(OC)nc2)c1]

To a solution of 2-(3,3-difluoropyrrolidin-1-yl)-4-(2-fluoro-5-methoxy-phenyl)pyridin-3-amine (0.0450 g, 0.125 mmol), 6-methoxypyridine-3-carboxylic acid (0.0288 g, 0.188 mmol), and 2-chloro-1-methyl-pyridin-1-ium iodide (0.128 g, 0.501 mmol) in tetrahydrofuran (1.63 mL) was added N,N-diisopropylethylamine (0.0858 mL, 0.501 mmol), and the mixture was stirred at 65° C. for 20 h. After cooling to ambient temperature, the mixture was diluted with saturated aqueous sodium bicarbonate (15 mL), and the aqueous phase was extracted with ethyl acetate (3×15 mL). The organic phase was dried over anhydrous sodium sulfate, filtered, and concentrated in vacuo. Purification of the residue by column chromatography, eluting with a gradient of 0-15% of methanol in dichloromethane, followed by preparative HPLC, eluting with a gradient of 50-60% of acetonitrile in water containing 10 mM of ammonium bicarbonate, afforded the title compound as a colorless solid (0.0100 g, 17% yield): 1H NMR (500 MHz; DMSO-d₆) δ 9.85 (s, 1H), 8.53 (d, J=2.1 Hz, 1H), 8.19 (d, J=5.0 Hz, 1H), 7.98 (dd, J=8.7, 2.5 Hz, 1H), 7.15 (t, J=9.2 Hz, 1H), 6.89 (t, J=7.0 Hz, 2H), 6.84 (d, J=5.7 Hz, 1H), 6.80 (d, J=5.5 Hz, 1H), 3.90 (s, 3H), 3.99-3.65 (m, 4H), 3.33 (s, 3H), 2.43 (ddd, J=20.9, 13.9, 7.0 Hz, 2H); MS (ES+) m/z 459.2 (M+1).

### Example 61

### Synthesis of N-(2-(3,3-difluoropyrrolidin-1-yl)-4-(3-methyl-1H-pyrazol-5-yl)pyridin-3-yl)-2-isopropylpyrimidine-5-carboxamide

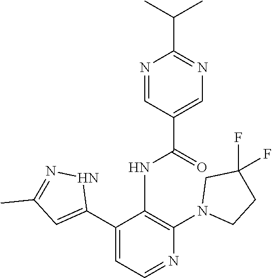 [SMILES: Cc1cc(-c2ccnc(N3CCC(F)(F)C3)c2NC(=O)c2cnc(C(C)C)nc2)[nH]n1]

To a solution of N-[2-(3,3-difluoropyrrolidin-1-yl)-4-iodo-3-pyridyl]-2-isopropyl-pyrimidine-5-carboxamide (0.080 g, 0.17 mmol), (3-methyl-1H-pyrazol-5-yl)boronic acid (0.032 g, 0.25 mmol), and potassium carbonate (0.070 g, 0.51 mmol) in degassed 1,4-dioxane (1.0 mL) and water (0.11 mL) was added [1,1′-Bis(diphenylphosphino)ferrocene]dichloropalladium(II), complex with dichloromethane (1:1) (0.014 g, 0.017 mmol) and the mixture was stirred at 90° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by preparative reverse phase HPLC, eluting with a gradient of 10-40% acetonitrile in water containing 0.5% formic acid, afforded the title compound as an off-white solid (0.025 g, 34% yield). ¹H-NMR (300 MHz; DMSO-d₆) δ 12.86 (s, 1H), 10.27 (s, 1H), 9.22 (s, 2H), 8.12 (d, J=5.1 Hz, 1H), 7.12 (d, J=5.1 Hz, 1H), 6.39 (s, 1H), 3.94-3.65 (m, 4H), 3.24 (dt, J=13.8, 6.9 Hz, 2H), 2.46-2.34 (m, 1H), 2.19 (s, 3H), 1.33 (d, J=6.9 Hz, 6H); MS (ESI+) m/z 428.2 (M+1).

### Example 62

### Synthesis of N-(2-(3,3-difluoropyrrolidin-1-yl)-4-(oxazol-5-yl)pyridin-3-yl)-2-isopropylpyrimidine-5-carboxamide

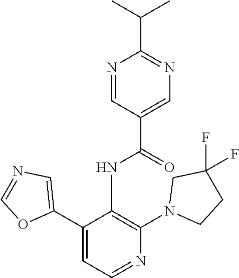 [SMILES: CC(C)c1ncc(C(=O)Nc2c(-c3cnco3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.080 g, 0.17 mmol), 5-(4,4,5,5-tetramethyl-[1,3,2]dioxaborolan-2-yl)-oxazole (0.049 g, 0.25 mmol), and potassium carbonate (0.070 g, 0.51 mmol) in degassed 1,4-dioxane (1.00 mL) and water (0.11 mL) was added [1,1′-bis(diphenylphosphino)ferrocene]dichloropalladium(II), complex with dichloromethane (1:1) (0.014 g, 0.017 mmol) and the mixture was stirred at 90° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by preparative reverse phase HPLC, eluting with a gradient of 5-65% acetonitrile in water containing 0.5% formic acid, afforded the title compound as a colorless solid (0.025 g, 36% yield). ¹H-NMR (300 MHz; DMSO-d₆): δ 10.53-10.49 (m, 1H), 9.27 (s, 2H), 8.55 (s, 1H), 8.24 (d, J=5.2 Hz, 1H), 7.57 (s, 1H), 7.17 (d, J=5.2 Hz, 1H), 3.93-3.71 (m, 4H), 3.25 (dt, J=13.9, 7.0 Hz, 1H), 2.46-2.37 (m, 2H), 1.34 (d, J=6.9 Hz, 6H); MS (ESI+) m/z 415.2 (M+1).

### Example 63

### Synthesis of 6-isopropyl-N-(2-morpholino-4-phenylpyridin-3-yl)nicotinamide

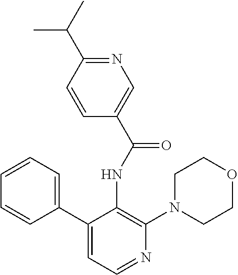 [SMILES: CC(C)c1ccc(C(=O)Nc2c(-c3ccccc3)ccnc2N2CCOCC2)cn1]

### Step 1. Preparation of 2-chloro-3-nitro-4-phenylpyridine

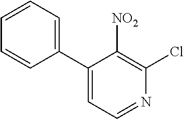 [SMILES: O=[N+]([O-])c1c(-c2ccccc2)ccnc1Cl]

A mixture of 2,4-dichloro-3-nitropyridine (3.00 g, 15.5 mmol) in dioxane (100 mL) and water (10 mL) was degassed with nitrogen for 10 minutes. To the reaction mixture was added phenylboronic acid (1.90 g, 15.5 mmol), dichloro 1,1′-bis(diphenylphosphino)ferrocene palladium (II) dichloromethane (1.32 g, 1.55 mmol), and potassium carbonate (3.22 g, 23.3 mmol). The reaction mixture was stirred at 60° C. for 4 h. After cooling to ambient temperature, the mixture was filtered through a bed of diatomaceous earth (i.e., Celite®) and diluted with ethyl acetate (150 mL). The combined filtrate was washed with saturated ammonium chloride (3×100 mL), dried over anhydrous magnesium sulfate, filtered and concentrated in vacuo. The residue was purified by column chromatography, eluting with a gradient of 5 to 45% ethyl acetate in heptane, to afford the title compound as a colorless solid (2.95 g, 81% yield): MS (ES+) m/z 235.0 (M+1).

### Step 2. Preparation of 4-(3-nitro-4-phenylpyridin-2-yl)morpholine

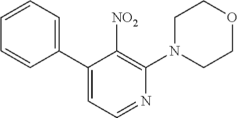 [SMILES: O=[N+]([O-])c1c(-c2ccccc2)ccnc1N1CCOCC1]

To a mixture of 2-chloro-3-nitro-4-phenylpyridine (0.500 g, 2.13 mmol) in anhydrous N,N-dimethylformamide (7.10 mL) was added potassium carbonate (0.884 g, 6.39 mmol) and morpholine (0.23 mL, 2.6 mmol). The reaction mixture was stirred at 50° C. for 30 minutes. After cooling to ambient temperature, the mixture was diluted with ethyl acetate (150 mL) and washed with saturated ammonium chloride (50 mL), water (4×50 mL), and brine (50 mL). The organic phase dried over anhydrous magnesium sulfate, filtered, and concentrated in vacuo. The residue was purified by column chromatography, eluting with a gradient of 5 to 45% ethyl acetate in heptane, to afford the title compound as a yellow oil (0.328 g, 54% yield): MS (ES+) m/z 286.2 (M+1).

### Step 3. Preparation of 2-morpholino-4-phenylpyridin-3-amine

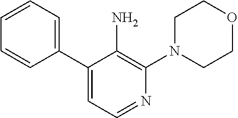 [SMILES: Nc1c(-c2ccccc2)ccnc1N1CCOCC1]

A mixture of 2,4-dichloro-3-nitropyridine (0.328 g, 1.15 mmol) in methanol (1.9 mL) and ethyl acetate (1.9 mL) was degassed with nitrogen for 10 minutes. To the reaction mixture was added 10% palladium on carbon (0.075 g). The reaction mixture was degassed with hydrogen and stirred at ambient temperature for 16 h. After cooling to ambient temperature, the mixture was filtered through a bed of diatomaceous earth (i.e., Celite®). The filter pad was washed with ethyl acetate (2×50 mL) and the combined filtrate was concentrated in vacuo. The residue was purified by column chromatography, eluting with a gradient of 5 to 60% ethyl acetate in heptane, to afford the title compound as a colorless solid (0.247 g, 84% yield): MS (ES+) m/z 256.2 (M+1).

### Step 4. Preparation of 6-isopropyl-N-(2-morpholino-4-phenylpyridin-3-yl)nicotinamide

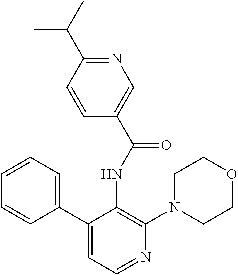 [SMILES: CC(C)c1ccc(C(=O)Nc2c(-c3ccccc3)ccnc2N2CCOCC2)cn1]

To a mixture of 2-morpholino-4-phenylpyridin-3-amine (0.050 g, 0.20 mmol) in anhydrous tetrahydrofuran (1.3 mL) was added N,N-diisopropylethylamine (0.34 mL, 2.0 mmol), 2-chloro-1-methylpyridinium iodide (0.200 g, 0.783 mmol), and isopropylnicotinic acid hydrochloride (0.063 g, 0.31 mmol). The reaction mixture was stirred at 65° C. for 20 h. After cooling to ambient temperature, the mixture was diluted in saturated ammonium chloride (50 mL) and extracted with ethyl acetate (3×100 mL). The combined extracts were washed with saturated ammonium chloride (3×50 mL), dried over anhydrous magnesium sulfate, filtered and concentrated in vacuo. The residue was purified by column chromatography, eluting with a gradient of 15 to 100% ethyl acetate in heptane, to afford the title compound as a colorless oil. Further purification of the residue by reverse-phase column chromatography, using a gradient of 10 to 65% acetonitrile in water containing 0.5% formic acid as eluent, afforded the title compound as a colorless solid (0.045 g, 56% yield): ¹H NMR (500 MHz, DMSO-d₆) δ 9.94 (s, 1H), 8.78-8.78 (m, 1H), 8.28 (d, J=5.0 Hz, 1H), 7.97 (dd, J=8.1, 1.8 Hz, 1H), 7.43 (d, J=7.3 Hz, 2H), 7.37 (t, J=6.7 Hz, 3H), 7.32 (t, J=7.2 Hz, 1H), 7.02 (d, J=5.0 Hz, 1H), 3.62 (t, J=4.5 Hz, 4H), 3.22-3.21 (m, 4H), 3.05 (sept, J=6.8 Hz, 1H), 1.23 (d, J=6.9 Hz, 6H); MS (ES+) m/z 403.2 (M+1).

### Examples 64-66

In a similar manner as described in EXAMPLE 63, utilizing the appropriately substituted starting materials and intermediates, the following compounds were prepared:

| | | | Amount (g) | |
|---|---|---|---|---|
| Example | | | Yield % | |
| No. | Structure | Name | MS (ES+) m/z | ¹H NMR |
| 64 | 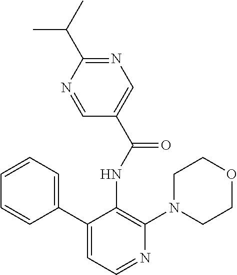 [SMILES: CC(C)c1ncc(C(=O)Nc2c(-c3ccccc3)ccnc2N2CCOCC2)cn1] | 2-isopropyl- N-(2- morpholino- 4- phenylpyridin- 3- yl)pyrimidine- 5- carboxamide | 0.034 g 34% 404.2 (M + 1) | (500 MHz, DMSO- d₆) δ 10.16 (s, 1H), 8.95 (s, 2H), 8.30 (d, J = 5.0 Hz, 1H), 7.43-7.38 (m, 4H), 7.35-7.33 (m, 1H), 7.04 (d, J = 5.0 Hz, 1H), 3.64 (t, J = 4.5 Hz, 4H), 3.23-3.16 (m, 5H), 1.29-1.25 (m, 6H) |
| 65 | 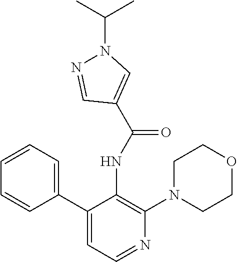 [SMILES: CC(C)n1cc(C(=O)Nc2c(-c3ccccc3)ccnc2N2CCOCC2)cn1] | 2-fluoro-4- isopropyl-N- (2- morpholino-4- phenylpyridin- 3-yl)benzamide | 0.023 g 31% 392.2 (M + 1) | (500 MHz, DMSO- d₆) δ 9.34 (s, 1H), 8.24 (d, J = 5.0 Hz, 1H), 8.17 (s, 1H), 7.87 (s, 1H), 7.44- 7.42 (m, 2H), 7.35 (t, J = 7.4 Hz, 2H), 7.30 (t, J = 7.2 Hz, 1H), 6.98 (d, J = 5.0 Hz, 1H), 4.48 (sept, J = 6.6 Hz, 1H), 3.60 (t, J = 4.5 Hz, 4H), 3.20-3.19 (m, 4H), 1.39 (d, J = 6.6 Hz, 6H) |
| 66 | 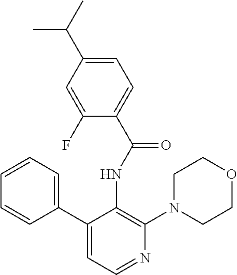 [SMILES: CC(C)c1ccc(C(=O)Nc2c(-c3ccccc3)ccnc2N2CCOCC2)c(F)c1] | 2-fluoro-4- isopropyl-N-(2- morpholino-4- phenylpyridin- 3-yl)benzamide | 0.029 g 39% 420.4 (M+1) | (300 MHz, DMSO- d₆) δ 9.70 (s, 1H), 8.27 (d, J = 5.0 Hz, 1H), 7.49-7.34 (m, 5H), 7.18-7.09 (m, 3H), 7.00 (d, J = 5.0 Hz, 1H), 3.69 (t, J = 4.5 Hz, 4H), 3.22 (t, J = 4.4 Hz, 4H), 2.90 (sept, J = 6.9 Hz, 1H), 1.18 (d, J = 6.9 Hz, 6H) |

### Example 67

### Synthesis of N-(2-(3,3-difluoroazetidin-1-yl)-4-phenylpyridin-3-yl)-2-isopropylpyrimidine-5-carboxamide

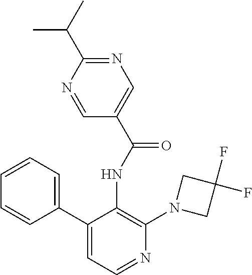 [SMILES: CC(C)c1ncc(C(=O)Nc2c(-c3ccccc3)ccnc2N2CC(F)(F)C2)cn1]

### Step 1. Preparation of 2-(3,3-difluoroazetidin-1-yl)-3-nitro-4-phenylpyridine

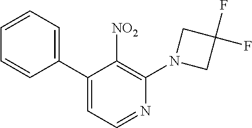 [SMILES: O=[N+]([O-])c1c(-c2ccccc2)ccnc1N1CC(F)(F)C1]

To a mixture of 2-chloro-3-nitro-4-phenylpyridine (0.500 g, 2.13 mmol) in anhydrous N,N-dimethylformamide (7.10 mL) was added potassium carbonate (0.884 g, 6.39 mmol) and 3,3-difluoroazetidine hydrochloride (0.663 g, 5.11 mmol). The reaction mixture was stirred at 50° C. for 3 h. After cooling to ambient temperature, the mixture was diluted in ethyl acetate (150 mL) and the organic phase was washed with saturated ammonium chloride (50 mL), water (4×50 mL), brine (50 mL), dried over anhydrous magnesium sulfate, filtered and concentrated in vacuo. The residue was purified by column chromatography, eluting with a gradient of 5 to 45% ethyl acetate in heptane, to afford the title compound as a yellow oil (0.426 g, 69% yield): MS (ES+) m/z 292.0 (M+1).

### Step 2. Preparation of 2-(3,3-difluoroazetidin-1-yl)-4-phenylpyridin-3-amine

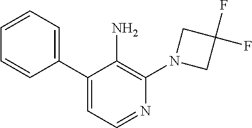 [SMILES: Nc1c(-c2ccccc2)ccnc1N1CC(F)(F)C1]

A mixture of 2-(3,3-difluoroazetidin-1-yl)-3-nitro-4-phenylpyridine (0.453 g, 1.55 mmol) in methanol (2.6 mL) and ethyl acetate (2.6 mL) was degassed with nitrogen for 10 minutes. To the reaction mixture was added 10% palladium on carbon (0.075 g). The reaction mixture was degassed with hydrogen and stirred at ambient temperature for 16 h. The mixture was filtered through a bed of diatomaceous earth (i.e., Celite®), the filter pad was washed with ethyl acetate (2×50 mL), and the combined filtrate was concentrated in vacuo. The residue was purified by column chromatography, eluting with a gradient of 5 to 35% ethyl acetate in heptane, to afford the title compound as a yellow solid (0.341 g, 84% yield): MS (ES+) m/z 262.0 (M+1).

### Step 3. Preparation of N-(2-(3,3-difluoroazetidin-1-yl)-4-phenylpyridin-3-yl)-2-isopropylpyrimidine-5-carboxamide

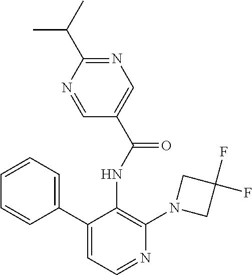 [SMILES: CC(C)c1ncc(C(=O)Nc2c(-c3ccccc3)ccnc2N2CC(F)(F)C2)cn1]

To a mixture of 2-(3,3-difluoroazetidin-1-yl)-4-phenylpyridin-3-amine (0.050 g, 0.20 mmol) in anhydrous tetrahydrofuran (1.3 mL) was added N,N-diisopropylethylamine (0.42 mL, 2.4 mmol), 2-chloro-1-methylpyridinium iodide (0.245 g, 0.960 mmol), and 2-isopropylpyrimidine-5-carboxylic acid (0.064 g, 0.38 mmol). The reaction mixture was stirred at 65° C. for 3 days. After cooling to ambient temperature, the mixture was diluted in ethyl acetate (100 mL) and washed with saturated ammonium chloride (35 mL), dried over anhydrous magnesium sulfate, filtered and concentrated in vacuo. The residue was purified by column chromatography, eluting with a gradient of 5 to 100% ethyl acetate in heptane, to afford the title compound as a colorless solid (0.024 g, 32% yield): ¹H NMR (500 MHz, DMSO-d₆) δ 10.14 (s, 1H), 8.97 (s, 2H), 8.22 (d, J=5.1 Hz, 1H), 7.42-7.37 (m, 4H), 7.34 (m, J=7.4, 4.9, 2.5 Hz, 1H), 6.90 (d, J=5.1 Hz, 1H), 4.51-4.46 (m, 2H), 4.38-4.32 (m, 2H), 3.18 (sept, J=6.9 Hz, 1H), 1.27 (d, J=6.9 Hz, 6H); MS (ES+) m/z 410.2 (M+1).

### Example 68

In a similar manner as described in EXAMPLE 67, utilizing the appropriately substituted starting materials and intermediates, the following compounds were prepared:

| | | | Amount (g) | |
|---|---|---|---|---|
| Example | | | Yield % | |
| No. | Structure | Name | MS (ES+) m/z | ¹H NMR |
| 68 | 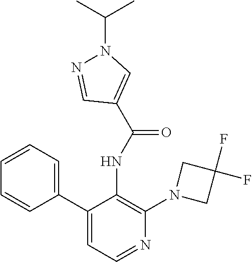 [SMILES: CC(C)n1cc(C(=O)Nc2c(-c3ccccc3)ccnc2N2CC(F)(F)C2)cn1] | N-(2-(3,3- difluoroazetidin- 1-yl)-4- phenylpyridin- 3-yl)-1- isopropyl-1H- pyrazole-4- carboxamide | 0.025 g 34% 398.2 (M + 1) | (500 MHz, DMSO- d₆) δ 9.40 (s, 1H), 8.19 (s, 1H), 8.17 (d, J = 5.1 Hz, 1H), 7.87 (s, 1H), 7.41 (d, J = 7.7 Hz, 2H), 7.38-7.31 (m, 3H), 6.87 (d, J = 5.1 Hz, 1H), 4.52-4.46 (m, 3H), 4.34-4.27 (m, 2H), 1.40 (d, J = 6.6 Hz, 6H) |

### Example 69

### Synthesis of 2-isopropyl-N-(4-phenyl-2-(2-oxa-6-azaspiro[3.3]heptan-6-yl)pyridin-3-yl)pyrimidine-5-carboxamide formic acid salt

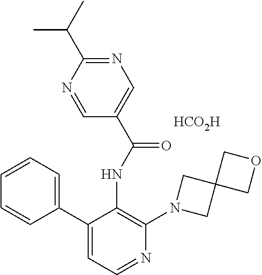 [SMILES: CC(C)c1ncc(C(=O)Nc2c(-c3ccccc3)ccnc2N2CC3(COC3)C2)cn1 ; "HCO2H"]

### Step 1. Preparation of 6-(3-nitro-4-phenylpyridin-2-yl)-2-oxa-6-azaspiro[3.3]heptane

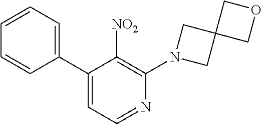 [SMILES: O=[N+]([O-])c1c(-c2ccccc2)ccnc1N1CC2(COC2)C1]

To a mixture of 2-chloro-3-nitro-4-phenylpyridine (0.500 g, 2.13 mmol) in anhydrous N,N-dimethylformamide (7.10 mL) was added potassium carbonate (0.884 g, 6.39 mmol) and 2-oxa-6-azaspiro[3.3]heptane oxalic acid (0.967 g, 5.11 mmol). The reaction mixture was stirred at 50° C. for 3 h. After cooling to ambient temperature, the mixture diluted in ethyl acetate (150 mL) and washed with saturated ammonium chloride (50 mL), water (4×50 mL), brine (50 mL), dried over anhydrous magnesium sulfate, filtered and concentrated in vacuo. The residue was purified by column chromatography, eluting with a gradient of 5 to 30% ethyl acetate in heptane, to afford the title compound as a yellow oil (0.453 g, 71% yield): MS (ES+) m/z 298.0 (M+1).

### Step 2. Preparation of 4-phenyl-2-(2-oxa-6-azaspiro[3.3]heptan-6-yl)pyridin-3-amine

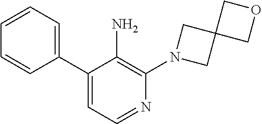 [SMILES: Nc1c(-c2ccccc2)ccnc1N1CC2(COC2)C1]

A mixture of 6-(3-nitro-4-phenylpyridin-2-yl)-2-oxa-6-azaspiro[3.3]heptane (0.453 g, 1.55 mmol) in methanol (2.6 mL) and ethyl acetate (2.6 mL) was degassed with nitrogen for 10 minutes. To the reaction mixture was added 10% palladium on carbon (0.075 g). The reaction mixture was degassed with hydrogen and stirred at ambient temperature for 16 h. After cooling to ambient temperature, the mixture was filtered through a bed of diatomaceous earth (i.e., Celite®). The filter pad was washed with ethyl acetate (2×50 mL) and the combined filtrate was concentrated in vacuo. The residue was purified by column chromatography, eluting with a gradient of 5 to 70% ethyl acetate in heptane, to afford the title compound as a yellow solid (0.259 g, 62% yield): MS (ES+) m/z 268.2 (M+1).

### Step 3. 2-isopropyl-N-(4-phenyl-2-(2-oxa-6-azaspiro[3.3]heptan-6-yl)pyridin-3-yl)pyrimidine-5-carboxamide formic acid salt

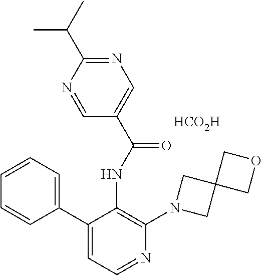 [SMILES: CC(C)c1ncc(C(=O)Nc2c(-c3ccccc3)ccnc2N2CC3(COC3)C2)cn1 ; "HCO2H"]

To a mixture of 4-phenyl-2-(2-oxa-6-azaspiro[3.3]heptan-6-yl)pyridin-3-amine (0.050 g, 0.20 mmol) in anhydrous tetrahydrofuran (1.3 mL) was added N,N-diisopropylethylamine (0.42 mL, 2.4 mmol), 2-chloro-1-methylpyridinium iodide (0.245 g, 0.960 mmol), and 2-isopropylpyrimidine-5-carboxylic acid (0.064 g, 0.38 mmol). The reaction mixture was stirred at 65° C. for 3 days. After cooling to ambient temperature, the mixture was diluted in ethyl acetate (100 mL) and the organic phase was washed with saturated ammonium chloride (35 mL), dried over anhydrous magnesium sulfate, filtered and concentrated in vacuo. The residue was purified by column chromatography, eluting with a gradient of 5 to 100% ethyl acetate in heptane, to afford the title compound as a colorless oil. Further purification of the residue by reverse-phase column chromatography, using a gradient of 10 to 55% acetonitrile in water containing 0.5% formic acid as eluent, afforded the title compound as a colorless solid (0.021 g, 27% yield): ¹H NMR (500 MHz, DMSO-d₆) δ 10.10 (s, 1H), 8.99 (s, 2H), 8.45 (s, 0.35H), 8.13 (d, J=5.0 Hz, 1H), 7.40-7.35 (m, 4H), 7.34-7.31 (m, 1H), 6.72 (d, J=5.0 Hz, 1H), 4.67 (s, 4H), 4.27-4.24 (m, 2H), 4.14-4.11 (m, 2H), 3.19 (sept, J=6.9 Hz, 1H), 1.29 (d, J=6.9 Hz, 6H); MS (ES+) 416.2 m/z (M+1).

### Example 70-72

In a similar manner as described in EXAMPLE 69, utilizing the appropriately substituted starting materials and intermediates, the following compounds were prepared:

| | | | Amount (g) | |
|---|---|---|---|---|
| Example | | | Yield % | |
| No. | Structure | Name | MS (ES+) m/z | ¹H NMR |
| 70 | 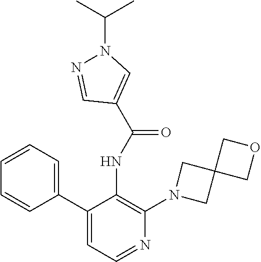 [SMILES: CC(C)n1cc(C(=O)Nc2c(-c3ccccc3)ccnc2N2CC3(COC3)C2)cn1] | 1-isopropyl- N-(4-phenyl- 2-(2-oxa-6- azaspiro[3.3] heptan-6- yl)pyridin-3- yl)-1H- pyrazole-4- carboxamide | 0.012 g 16% 404.2 (M + 1) | (500 MHz, DMSO-d₆) δ 9.29 (s, 1H), 8.17 (s, 1H), 8.08 (d, J = 5.1 Hz, 1H), 7.88 (s, 1H), 7.40-7.38 (m, 2H), 7.35-7.27 (m, 3H), 6.68 (d, J = 5.1 Hz, 1H), 4.65 (s, 4H), 4.49 (sept, J = 6.6 Hz, 1H), 4.21 (s, 2H), 4.09 (s, 2H), 1.41 (d, J = 6.7 Hz, 6H) |
| 71 | 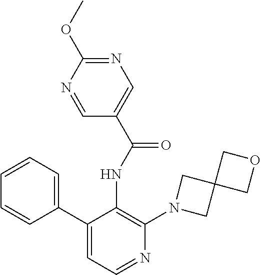 [SMILES: COc1ncc(C(=O)Nc2c(-c3ccccc3)ccnc2N2CC3(COC3)C2)cn1] | 2-methoxy- N-(4-phenyl- 2-(2-oxa-6- azaspiro[3.3] heptan-6- yl)pyridin-3- yl)pyrimidine- 5- carboxamide | 0.034 g 45% 404.2 (M + 1) | (500 MHz, DMSO-d₆) δ 9.93 (s, 1H), 8.91 (s, 2H), 8.12 (d, J = 5.0Hz, 1H), 7.39-7.34 (m, 4H), 7.33-7.29 (m, 1H), 6.72 (d, J = 5.0 Hz, 1H), 4.66 (s, 4H), 4.24 (d, J = 8.4 Hz, 2H), 4.11 (d, J = 8.0 Hz, 2H), 3.98 (s, 3H) |
| 72 | 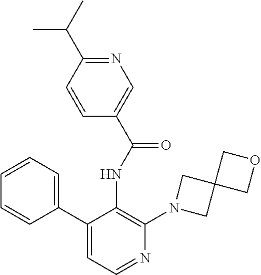 [SMILES: CC(C)c1ccc(C(=O)Nc2c(-c3ccccc3)ccnc2N2CC3(COC3)C2)cn1] | 6-isopropyl- N-(4-phenyl- 2-(2-oxa-6- azaspiro[3.3] heptan-6- yl)pyridin-3- yl)nicotinamide | 0.049 g 63% 415.2 (M + 1) | (500 MHz, DMSO-d₆) δ 9.85 (s, 1H), 8.82 (dd, J = 2.3, 0.7 Hz, 1H), 8.11 (d, J = 5.0 Hz, 1H), 8.01 (dd, J = 8.1, 2.4 Hz, 1H), 7.41-7.30 (m, 6H), 6.71 (d, J = 5.1 Hz, 1H), 4.66 (s, 4H), 4.26-4.08 (m, 4H), 3.06 (sept, J = 6.9 Hz, 1H), 1.24 (d, J = 6.9 Hz, 6H) |

### Example 73

### Synthesis of (R)-2-isopropyl-N-(4-phenyl-2-(2-(trifluoromethyl)pyrrolidin-1-yl)pyridin-3-yl)pyrimidine-5-carboxamide

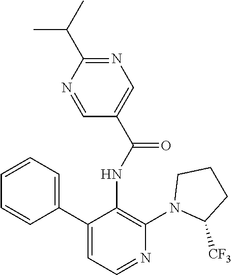 [SMILES: CC(C)c1ncc(C(=O)Nc2c(-c3ccccc3)ccnc2N2CCC[C@@H]2C(F)(F)F)cn1]

### Step 1. (R)-3-nitro-4-phenyl-2-(2-(trifluoromethyl)pyrrolidin-1-yl)pyridine

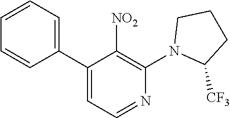 [SMILES: O=[N+]([O-])c1c(-c2ccccc2)ccnc1N1CCC[C@@H]1C(F)(F)F]

To a mixture of 2-chloro-3-nitro-4-phenylpyridine (0.505 g, 2.15 mmol) in anhydrous dimethylsulfoxide (3.55 mL) was added N,N-diisopropylethylamine (1.55 mL, 8.61 mmol) and 2-(R)-2-trifluoromethylpyrrolidine (0.599 g, 2.15 mmol). The reaction mixture was stirred at 125° C. for 16 h. After cooling to ambient temperature, the mixture diluted with saturated ammonium chloride (50 mL) and extracted with ethyl acetate (3×100 mL). The combined organic layers were washed with saturated ammonium chloride (50 mL), dried over anhydrous magnesium sulfate, filtered and concentrated in vacuo. The residue was purified by column chromatography, eluting with a gradient of 5 to 30% ethyl acetate in heptane, to afford the title compound as a yellow oil (0.444 g, 61% yield): MS (ES+) m/z 338.2 (M+1).

### Step 2. Preparation of (R)-4 phenyl-2-(2-(trifluoromethyl)pyrrolidin-1-yl)pyridin-3-amine

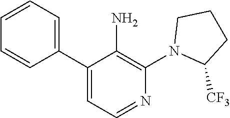 [SMILES: Nc1c(-c2ccccc2)ccnc1N1CCC[C@@H]1C(F)(F)F]

A mixture of (R)-3-nitro-4-phenyl-2-(2-(trifluoromethyl)pyrrolidin-1-yl)pyridine (0.444 g, 1.32 mmol) in methanol (2.6 mL) and ethyl acetate (2.6 mL) was degassed with nitrogen for 10 minutes. To the reaction mixture was added 10% palladium on carbon (0.090 g). The reaction mixture was degassed with hydrogen and stirred at ambient temperature for 16 h. The mixture was filtered through a bed of diatomaceous earth (i.e., Celite®), the filter pad was washed with ethyl acetate (2×50 mL), and the combined filtrate was concentrated in vacuo. The residue was purified by column chromatography, eluting with a gradient of 5 to 35% ethyl acetate in heptane, to afford the title compound as a yellow oil (0.329 g, 81% yield): MS (ES+) m/z 308.2 (M+1).

### Step 3. (R)-2-isopropyl-N-(4-phenyl-2-(2-(trifluoromethyl)pyrrolidin-1-yl)pyridin-3-yl)pyrimidine-5-carboxamide

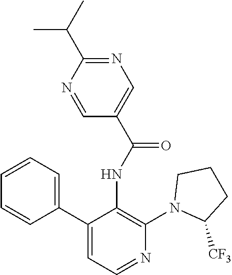 [SMILES: CC(C)c1ncc(C(=O)Nc2c(-c3ccccc3)ccnc2N2CCC[C@@H]2C(F)(F)F)cn1]

To a mixture of (R)-4-phenyl-2-(2-(trifluoromethyl)pyrrolidin-1-yl)pyridin-3-amine (0.050 g, 0.16 mmol) in anhydrous tetrahydrofuran (1.3 mL) was added N,N-diisopropylethylamine (0.36 mL, 2.1 mmol), 2-chloro-1-methylpyridinium iodide (0.214 g, 0.836 mmol), and 2-isopropylpyrimidine-5-carboxylic acid (0.056 g, 0.33 mmol). The reaction mixture was stirred at 65° C. for 2 h. After cooling to ambient temperature, the mixture was diluted in ethyl acetate (100 mL), the organic phase was washed with saturated ammonium chloride (35 mL), dried over anhydrous magnesium sulfate, filtered and concentrated in vacuo. The residue was purified by column chromatography, eluting with a gradient of 10 to 100% ethyl acetate in heptane, to afford the title compound as a colorless oil. Further purification of the residue by reverse-phase column chromatography, using a gradient of 10 to 90% acetonitrile in water containing 0.5% formic acid as eluent afforded the title compound as a colorless solid (0.030 g, 32% yield): ¹H NMR (500 MHz, DMSO-d₆) δ 10.16 (s, 1H), 8.94 (s, 2H), 8.21 (d, J=5.0 Hz, 1H), 7.43-7.35 (m, 4H), 7.34-7.29 (m, 1H), 6.91 (d, J=4.9 Hz, 1H), 5.69-5.64 (m, 1H), 3.66 (s, 1H), 3.42-3.24 (m, 1H), 3.17 (sept, J=7.1 Hz, 1H), 2.16-2.08 (m, 1H), 1.98-1.91 (m, 2H), 1.89-1.82 (m, 1H), 1.27 (d, J=6.9 Hz, 6H); MS (ESI) 456.2 m/z (M+1).

### Example 74

### Synthesis of 2-isopropyl-N-(4-phenyl-2-(pyrrolidin-1-yl)pyridin-3-yl)pyrimidine-5-carboxamide

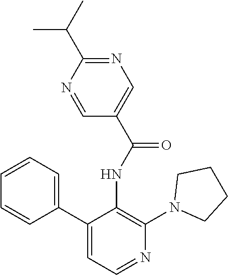 [SMILES: CC(C)c1ncc(C(=O)Nc2c(-c3ccccc3)ccnc2N2CCCC2)cn1]

### Step 1. Preparation of 3-nitro-4-phenyl-2-(pyrrolidin-1-yl)pyridine

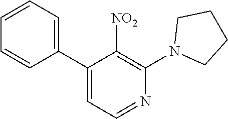 [SMILES: O=[N+]([O-])c1c(-c2ccccc2)ccnc1N1CCCC1]

To a mixture of 2-chloro-3-nitro-4-phenylpyridine (0.629 g, 2.68 mmol) in anhydrous dimethylsulfoxide (9.0 mL) was added potassium carbonate (1.11 g, 8.05 mmol) and pyrrolidine (0.45 mL, 5.4 mmol). The reaction mixture was stirred at ambient temperature for 16 h at ambient temperature. The mixture diluted with saturated ammonium chloride (50 mL), extracted with ethyl acetate (3×100 mL), and the combined organic layers were dried over anhydrous magnesium sulfate, filtered and concentrated in vacuo. The residue was purified by column chromatography, eluting with a gradient of 5 to 30% ethyl acetate in heptane, to afford the title compound as a yellow oil (0.716 g, 99% yield): MS (ES+) m/z 270.0 (M+1).

### Step 2. Preparation of 4-phenyl-2-(pyrrolidin-1-yl)pyridin-3-amine

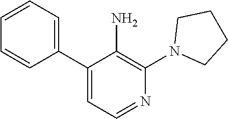 [SMILES: Nc1c(-c2ccccc2)ccnc1N1CCCC1]

A mixture of 3-nitro-4-phenyl-2-(pyrrolidin-1-yl)pyridine (0.716 g, 2.66 mmol) in methanol (2.6 mL) and ethyl acetate (2.6 mL) was degassed with nitrogen for 10 minutes. To the reaction mixture was added 10% palladium on carbon (0.095 g). The reaction mixture was degassed with hydrogen and stirred at ambient temperature for 16 h. The mixture was filtered through a bed of diatomaceous earth (i.e., Celite®), the filter pad was washed with ethyl acetate (2×50 mL), and the combined filtrate was concentrated in vacuo. The residue was purified by column chromatography, eluting with a gradient of 5 to 35% ethyl acetate in heptane, to afford the title compound as a yellow oil (0.521 g, 82% yield): MS (ES+) m/z 240.2 (M+1).

### Step 3. 2-isopropyl-N-(4-phenyl-2-(pyrrolidin-1-yl)pyridin-3-yl)pyrimidine-5-carboxamide

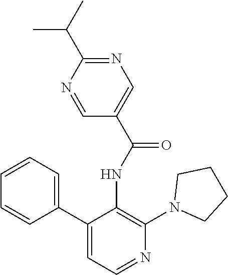 [SMILES: CC(C)c1ncc(C(=O)Nc2c(-c3ccccc3)ccnc2N2CCCC2)cn1]

To a mixture of 4-phenyl-2-(pyrrolidin-1-yl)pyridin-3-amine (0.050 g, 0.16 mmol) in anhydrous tetrahydrofuran (1.3 mL) was added N,N-diisopropylethylamine (0.36 mL, 2.1 mmol), 2-chloro-1-methylpyridinium iodide (0.214 g, 0.836 mmol), and 2-isopropylpyrimidine-5-carboxylic acid (0.056 g, 0.33 mmol). The reaction mixture was stirred at 65° C. for 2 h. After cooling to ambient temperature, the mixture was diluted in ethyl acetate (100 mL), the organic phase as washed with saturated ammonium chloride (35 mL), dried over anhydrous magnesium sulfate, filtered and concentrated in vacuo. The residue by reverse-phase column chromatography, using a gradient of 10 to 55% acetonitrile in water containing 0.5% formic acid as eluent, to afford the title compound as a colorless solid (0.036 g, 45% yield): ¹H NMR (500 MHz, DMSO-d₆) δ 10.09 (s, 1H), 8.91 (s, 2H), 8.11 (d, J=4.9 Hz, 1H), 7.36 (m, 4H), 7.32-7.30 (m, 1H), 6.63 (d, J=4.9 Hz, 1H), 3.60-3.38 (m, 4H), 3.16 (sept, J=6.9 Hz, 1H), 1.85-1.77 (m, 4H), 1.26 (d, J=6.9 Hz, 6H); MS (ES+) m/z 388.2 (M+1).

### Example 75

### Synthesis of N-(2-(6,6-difluoro-3-azabicyclo[3.1.0]hexan-3-yl)-4-phenylpyridin-3-yl)-2-isopropylpyrimidine-5-carboxamide

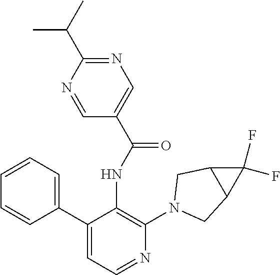 [SMILES: CC(C)c1ncc(C(=O)Nc2c(-c3ccccc3)ccnc2N2CC3C(C2)C3(F)F)cn1]

### Step 1. Preparation of 2-chloro-4-phenylpyridin-3-amine

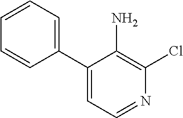 [SMILES: Nc1c(-c2ccccc2)ccnc1Cl]

To a mixture of 2-chloro-3-nitro-4-phenylpyridine (6.00 g, 25.6 mmol) in ethanol (51 mL) and water (51 mL) was added ammonium chloride (13.7 g, 256 mmol) and iron (7.14 g, 128 mmol). The reaction mixture was stirred at 80° C. for 1.5 h. After cooling to ambient temperature, the mixture was diluted in ethyl acetate (600 mL) and filtered through a bed of diatomaceous earth (i.e., Celite®). The filtrate was washed with saturated sodium bicarbonate (2×200 mL), brine (200 mL), dried over anhydrous magnesium sulfate, filtered and concentrated in vacuo, to afford the title compound as a colorless solid (5.30 g, 101% yield): MS (ES+) m/z 206.0 (M+1), 208.0 (M+1).

### Step 2. Preparation of N-(2-chloro-4-phenylpyridin-3-yl)-2-isopropylpyrimidine-5-carboxamide

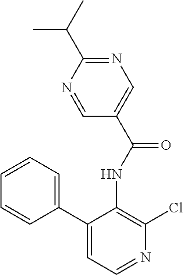 [SMILES: CC(C)c1ncc(C(=O)Nc2c(-c3ccccc3)ccnc2Cl)cn1]

To a mixture of 2-chloro-4-phenylpyridin-3-amine (2.50 g, 12.2 mmol) in anhydrous tetrahydrofuran (61 mL) and pyridine (9.80 mL, 122 mmol) was added 2-chloro-1-methylpyridinium iodide (9.36 g, 36.6 mmol) and 2-isopropylpyrimidine-5-carboxylic acid (2.23 g, 13.4 mmol). The reaction mixture was stirred at 65° C. for 2 days. After cooling to ambient temperature, the mixture diluted in saturated ammonium chloride (100 mL), extracted with ethyl acetate (2×200 mL), and the combined organic phase was washed with saturated ammonium chloride (100 mL), dried over anhydrous magnesium sulfate, filtered and concentrated in vacuo. The residue was purified by column chromatography, eluting with a gradient of 0 to 75% ethyl acetate in heptane, to afford the title compound as a yellow solid (2.85 g, 66% yield): ¹H NMR (300 MHz, CDCl₃) δ 8.96 (s, 2H), 8.41 (d, J=5.0 Hz, 1H), 7.58 (s, 1H), 7.41 (s, 5H), 7.32 (d, J=5.0 Hz, 1H), 3.27 (sept, J=6.8 Hz, 1H), 1.35 (d, J=6.9 Hz, 6H); MS (ES+) m/z 353.0 (M+1), 355.0 (M+1).

### Step 3. N-(2-(6,6-difluoro-3-azabicyclo[3.1.0]hexan-3-yl)-4-phenylpyridin-3-yl)-2-isopropylpyrimidine-5-carboxamide

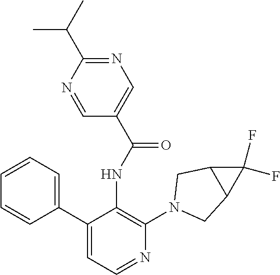 [SMILES: CC(C)c1ncc(C(=O)Nc2c(-c3ccccc3)ccnc2N2CC3C(C2)C3(F)F)cn1]

To a mixture of N-(2-chloro-4-phenylpyridin-3-yl)-2-isopropylpyrimidine-5-carboxamide (0.066 g, 0.19 mmol) in anhydrous 1,4-dioxane (1.9 mL) was added potassium tert-butoxide (0.104 g, 0.930 mmol), [1,3-bis(2,6-diisopropylphenyl)imidazol-2-ylidene](3-chloropyridyl)palladium(II) dichloride (0.040 g, 0.056 mmol), and 6,6-difluoro-3-azabicyclo[3.1.0]hexane hydrochloride (0.058 g, 0.37 mmol). The reaction mixture was degassed with nitrogen for 10 minutes, then was stirred at 100° C. for 24 h. The reaction mixture was cooled to ambient temperature and to the reaction mixture was added 1,3-bis(2,6-diisopropylphenyl)imidazol-2-ylidene](3-chloropyridyl)palladium(II) dichloride (0.040 g, 0.056 mmol). The reaction mixture was stirred at 100° C. for 3 d under an atmosphere of nitrogen. The reaction mixture was cooled to ambient temperature and to the reaction mixture was added potassium tert-butoxide (0.104 g, 0.930 mmol), 6,6-difluoro-3-azabicyclo[3.1.0]hexane hydrochloride (0.058 g, 0.37 mmol) and the reaction mixture was stirred at 100° C. for 18 h. After cooling to ambient temperature, the mixture was diluted with saturated ammonium chloride (50 mL), extracted with ethyl acetate (3×100 mL). The combined organic phase was dried over anhydrous magnesium sulfate, filtered and concentrated in vacuo. The residue was purified by column chromatography, eluting with a gradient of 20 to 100% ethyl acetate in heptane, to afford a colorless solid. Further purification of the residue by reverse-phase column chromatography, eluting with a gradient of 10 to 75% acetonitrile in water containing 0.5% formic acid as eluent, afforded the title compound as a colorless solid (0.015 g, 18% yield): ¹H NMR (300 MHz, DMSO-d₆) δ 10.20-10.09 (m, 1H), 8.92 (s, 2H), 8.15 (d, J=5.0 Hz, 1H), 7.41-7.28 (m, 5H), 6.69 (d, J=5.0 Hz, 1H), 5.43-5.24 (m, 1H), 3.91-3.52 (m, 4H), 3.17 (sept, J=6.9 Hz, 1H), 2.24-1.88 (m, 2H), 1.26 (d, J=6.9 Hz, 6H); MS (ES+) m/z 436.2 (M+1).

### Example 76

### Synthesis of (R)-N-(2-(3-fluoropyrrolidin-1-yl)-4-phenylpyridin-3-yl)-2-isopropylpyrimidine-5-carboxamide

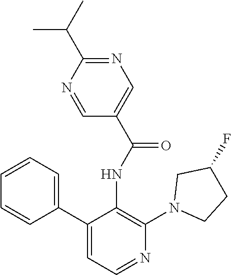 [SMILES: CC(C)c1ncc(C(=O)Nc2c(-c3ccccc3)ccnc2N2CC[C@@H](F)C2)cn1]

To a mixture of N-(2-chloro-4-phenylpyridin-3-yl)-2-isopropylpyrimidine-5-carboxamide (0.075 g, 0.21 mmol) in anhydrous 1,4-dioxane (2.1 mL) was added potassium tert-butoxide (0.143 g, 0.1.27 mmol), [1,3-bis(2,6-diisopropylphenyl)imidazol-2-ylidene](3-chloropyridyl)palladium(II) dichloride (0.058 g, 0.085 mmol), (R)-3-fluoropyrrolidine hydrochloride (0.107 g, 0.850 mmol). The reaction mixture was degassed with nitrogen for 10 minutes, then was stirred at 110° C. for 18 h. After cooling to ambient temperature, the mixture was diluted in ethyl acetate (150 mL) and filtered through a pad of diatomaceous earth (i.e., Celite®). The filtrate was washed with saturated ammonium chloride (2×50 mL), dried over anhydrous magnesium sulfate, filtered and concentrated in vacuo. The residue was purified by column chromatography, eluting with a gradient of 25 to 100% ethyl acetate in heptane, to afford the title compound as a colorless solid (0.027 g, 31% yield): ¹H NMR (300 MHz, DMSO-d₆) δ 510.18-10.11 (m, 1H), 8.92 (s, 2H), 8.15 (d, J=5.0 Hz, 1H), 7.41-7.28 (m, 5H), 6.69 (d, J=5.0 Hz, 1H), 5.43-5.24 (m, 1H), 3.91-3.52 (m, 4H), 3.17 (sept, J=6.9 Hz, 1H), 2.27-1.87 (m, 2H), 1.26 (d, J=6.9 Hz, 6H); MS (ES+) m/z 406.2 (M+1).

### Example 77

In a similar manner as described in EXAMPLE 76, utilizing the appropriately substituted starting materials and intermediates, the following compounds were prepared:

| | | | Amount (g) | |
|---|---|---|---|---|
| Example | | | Yield % | |
| No. | Structure | Name | MS (ES+) m/z | ¹H NMR |
| 77 | 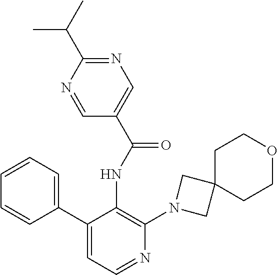 [SMILES: CC(C)c1ncc(C(=O)Nc2c(-c3ccccc3)ccnc2N2CC3(CCOCC3)C2)cn1] | 2-isopropyl- N-(4-phenyl- 2-(7-oxa-2- azaspiro [3.5] nonan-2- yl)pyridin-3- yl)pyrimidine- 5- carboxamide | 0.008 g 8% 444.2 (M + 1) | (300 MHz, DMSO-d₆) δ 10.04 (s, 1H), 8.95 (s, 2H), 8.12 (d, J = 5.0 Hz, 1H), 7.38-7.29 (m, 5H), 6.68 (d, J = 5.0 Hz, 1H), 3.90-3.71 (m, 4H), 3.51-3.48 (m, 4H), 3.18 (sept, J = 6.9 Hz, 1H), 1.67 (t, J = 5.0 Hz, 4H), 1.27 (d, J = 6.9 Hz, 6H) |

### Example 78

### Synthesis of 1-cyclobutyl-N-(4-(2-fluorophenyl)-2-(2-oxa-6-azaspiro[3.3]heptan-6-yl)pyridin-3-yl)-1H-pyrazole-4-carboxamide

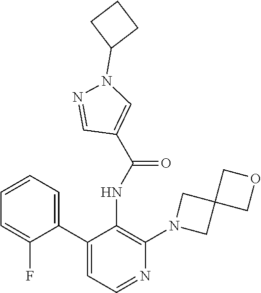 [SMILES: O=C(Nc1c(-c2ccccc2F)ccnc1N1CC2(COC2)C1)c1cnn(C2CCC2)c1]

### Step 1. Preparation of 2-chloro-4-(2-fluorophenyl)-3-nitropyridine

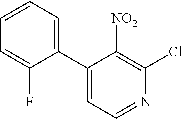 [SMILES: O=[N+]([O-])c1c(-c2ccccc2F)ccnc1Cl]

A mixture of 2,4-dichloro-3-nitropyridine (10.00 g, 51.82 mmol) in dioxane (100 mL) and water (35 mL) was degassed with nitrogen for 10 minutes. To the reaction mixture was added 2-fluorophenylboronic acid (7.98 g, 57.0 mmol), dichloro 1,1-bis(diphenylphosphino)ferrocene palladium (II) dichloromethane (3.29 g, 3.89 mmol), and potassium carbonate (10.74 g, 77.7 mmol). The reaction mixture was stirred at 60° C. for 8 hours. After cooling to ambient temperature, the mixture was filtered through a bed of diatomaceous earth (i.e., Celite®) and diluted in ethyl acetate (150 mL). The combined filtrate was washed with saturated ammonium chloride (2×100 mL), dried over anhydrous magnesium sulfate, filtered and concentrated in vacuo. The residue was purified by column chromatography, eluting with a gradient of 0 to 30% ethyl acetate in heptanes, to afford the title compound as a white solid (9.95 g, 76% yield): ¹H NMR (300 MHz, CDCl₃) δ 8.60 (d, J=5.0 Hz, 1H), 7.55-7.47 (m, 1H), 7.43 (dd, J=5.0, 1.3 Hz, 1H), 7.35-7.19 (m, 3H); MS (ES+) m/z 253.0 (M+1).

### Step 2. Preparation of 6-(4-(2-fluorophenyl)-3-nitropyridin-2-yl)-2-oxa-6-azaspiro[3.3]heptane

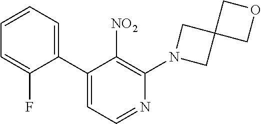 [SMILES: O=[N+]([O-])c1c(-c2ccccc2F)ccnc1N1CC2(COC2)C1]

To a mixture of 2-chloro-4-(2-fluorophenyl)-3-nitropyridine (4.00 g, 15.8 mmol) in N-methyl-2-pyrrolidone (53 mL) was added N,N-diisopropylethylamine (14 mL, 79 mmol) and 2-oxa-6-azaspiro[3.3]heptane oxalic acid (3.59 g, 19.0 mmol). The reaction mixture was stirred at 50° C. for 4 h. After cooling to ambient temperature, the mixture diluted in saturated ammonium chloride (200 mL) and extracted with ethyl acetate (3×200 mL). The combined organic phase was washed with saturated ammonium chloride (2×200 mL), dried over anhydrous magnesium sulfate, filtered and concentrated in vacuo. The residue was purified by column chromatography, eluting with a gradient of 5 to 100% ethyl acetate in heptane, to afford the title compound as a colorless solid (1.458 g, 29% yield): MS (ES+) m/z 316.2 (M+1).

### Step 3. Preparation of 4-(2-fluorophenyl)-2-(2-oxa-6-azaspiro[3.3]heptan-6-yl)pyridin-3-amine

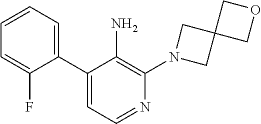 [SMILES: Nc1c(-c2ccccc2F)ccnc1N1CC2(COC2)C1]

A mixture of 6-(4-(2-fluorophenyl)-3-nitropyridin-2-yl)-2-oxa-6-azaspiro[3.3]heptane (1.458 g, 4.623 mmol) in methanol (7.7 mL) and ethyl acetate (7.7 mL) was degassed with nitrogen for 10 minutes. To the reaction mixture was added 10% palladium on carbon (0.245 g). The reaction mixture was degassed with hydrogen and stirred at ambient temperature for 16 h. After cooling to ambient temperature, the mixture was filtered through a bed of diatomaceous earth (i.e., Celite®). The filter pad was washed with ethyl acetate (2×100 mL) and the combined filtrate was concentrated in vacuo. The residue was purified by column chromatography, eluting with a gradient of 10 to 100% ethyl acetate in heptane, to afford the title compound as a brown solid (0.741 g, 56% yield): MS (ES+) m/z 286.2 (M+1).

### Step 4. Preparation of 1-cyclobutyl-N-(4-(2-fluorophenyl)-2-(2-oxa-6-azaspiro[3.3]heptan-6-yl)pyridin-3-yl)-1H-pyrazole-4-carboxamide

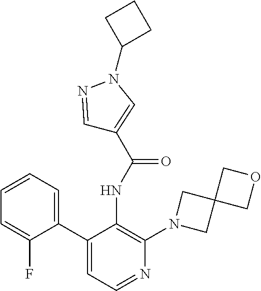 [SMILES: O=C(Nc1c(-c2ccccc2F)ccnc1N1CC2(COC2)C1)c1cnn(C2CCC2)c1]

To a mixture of 4-(2-fluorophenyl)-2-(2-oxa-6-azaspiro[3.3]heptan-6-yl)pyridin-3-amine (0.050 g, 0.20 mmol) in anhydrous tetrahydrofuran (1.75 mL) was added N,N-diisopropylethylamine (0.31 mL, 1.75 mmol), 2-chloro-1-methylpyridinium iodide (0.224 g, 0.876 mmol), 1-cyclobutyl-1H-pyrazole-4-carboxylic acid (0.087 g, 0.53 mmol). The reaction mixture was stirred at 65° C. for 18 h. After cooling to ambient temperature, the mixture was diluted in ethyl acetate (100 mL) and washed with saturated ammonium chloride (2×35 mL), dried over anhydrous magnesium sulfate, filtered and concentrated in vacuo. The residue was purified by reverse-phase column chromatography, eluting with a gradient of preparative reverse-phase HPLC, to afford the title compound as a colorless oil. Further purification of the residue by reverse-phase column chromatography, using a gradient of 10 to 35% acetonitrile in water containing 0.5% formic acid as eluent afforded the title compound as a colorless solid (0.023 g, 30% yield): ¹H NMR (300 MHz, DMSO-d₆) δ 9.31 (s, 1H), 8.19 (d, J=0.4 Hz, 1H), 8.09 (d, J=5.0 Hz, 1H), 7.88 (s, 1H), 7.37-7.10 (m, 4H), 6.68 (dd, J=5.1, 1.2 Hz, 1H), 4.82 (quintet, J=8.2 Hz, 1H), 4.65-4.63 (m, 4H), 4.16-4.13 (m, 4H), 2.44-2.34 (m, 4H), 1.80-1.71 (m, 2H); MS (ES+) 434.2 m/z (M+1).

### Example 79-84

In a similar manner as described in EXAMPLE 78, utilizing the appropriately substituted starting materials and intermediates, the following compounds were

| | | | Amount (g) | |
|---|---|---|---|---|
| Example | | | Yield % | |
| No. | Structure | Name | MS (ES+) m/z | ¹H NMR |
| 79 | 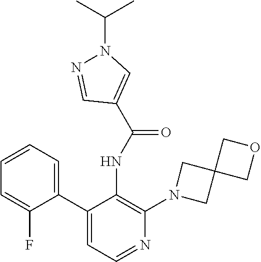 [SMILES: CC(C)n1cc(C(=O)Nc2c(-c3ccccc3F)ccnc2N2CC3(COC3)C2)cn1] | N-(4-(2- fluorophenyl)- 2-(2-oxa-6- azaspiro[3.3] heptan-6- yl)pyridin-3- yl)-1- isopropyl- 1H-pyrazole- 4- carboxamide | 0.016 g 14% 422.4 (M + 1) | (300 MHz, DMSO- d₆) δ 9.31 (s, 1H), 8.16 (d, J = 0.4 Hz, 1H), 8.08 (d, J = 5.2 Hz, 1H), 7.86 (d, J = 0.6 Hz, 1H), 7.38-7.11 (m, 4H), 6.70 (dd, J = 5.2, 1.1 Hz, 1H), 4.65 (s, 4H), 4.47 (sept, J = 6.6 Hz, 1H), 4.18 (s, 4H), 1.40 (d, J = 6.6 Hz, 6H) |
| 80 | 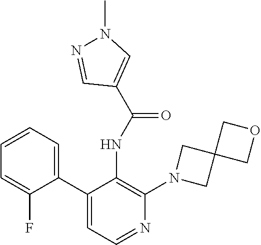 [SMILES: Cn1cc(C(=O)Nc2c(-c3ccccc3F)ccnc2N2CC3(COC3)C2)cn1] | N-(4-(2- fluorophenyl)- 2-(2-oxa-6- azaspiro[3.3] heptan-6- yl)pyridin-3- yl)-1-methyl- 1H-pyrazole- 4- carboxamide | 0.022 g 32% 394.0 (M + 1) | (300 MHz, DMSO- d₆) δ 9.29 (s, 1H), 8.08 (t, J = 2.5 Hz, 2H), 7.81 (d, J = 0.7 Hz, 1H), 7.37- 7.09 (m, 4H), 6.68 (dd, J = 5.1, 1.3 Hz, 1H), 4.64 (s, 4H), 4.15 (s, 4H), 3.82 (s, 3H) |
| 81 | 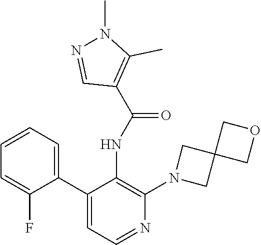 [SMILES: Cc1c(C(=O)Nc2c(-c3ccccc3F)ccnc2N2CC3(COC3)C2)cnn1C] | N-(4-(2- fluorophenyl)- 2-(2-oxa-6- azaspiro[3.3] heptan-6- yl)pyridin-3- yl)-1,5- dimethyl-1H- pyrazole-4- carboxamide | 0.024 g 33% 408.2 (M + 1) | (300 MHz, DMSO- d₆) δ 9.15 (s, 1H), 8.07 (d, J = 5.0 Hz, 1H), 7.80 (s, 1H), 7.37-7.27 (m, 2H), 7.20 (ddd, J = 10.0, 8.6, 1.2 Hz, 1H), 7.16-7.10 (m, 1H), 6.67 (dd, J = 5.0, 1.3 Hz, 1H), 4.65 (s, 4H), 4.16 (s, 4H), 3.68 (s, 3H), 2.30 (s, 3H) |
| 82 | 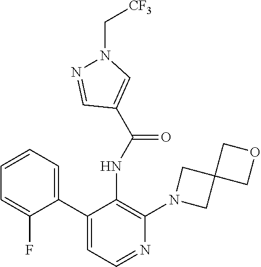 [SMILES: O=C(Nc1c(-c2ccccc2F)ccnc1N1CC2(COC2)C1)c1cnn(CC(F)(F)F)c1] | N-(4-(2- fluorophenyl)- 2-(2-oxa-6- azaspiro[3.3] heptan-6- yl)pyridin-3- yl)-1-(2,2,2- trifluoroethyl)-1H- pyrazole-4- carboxamide | 0.018 g 22% 462.2 (M + 1) | (300 MHz, DMSO- d₆) δ 9.52 (s, 1H), 8.25 (s, 1H), 8.09 (d, J = 5.2 Hz, 1H), 7.97 (d, J = 0.6 Hz, 1H), 7.38-7.11 (m, 4H), 6.73-6.71 (m, 1H), 5.18 (q, J = 9.2 Hz, 2H), 4.65 (s, 4H), 4.19 (s, 4H) |
| 83 | 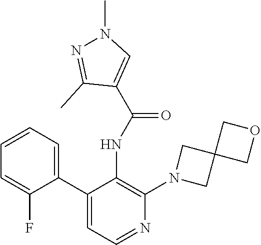 [SMILES: Cc1nn(C)cc1C(=O)Nc1c(-c2ccccc2F)ccnc1N1CC2(COC2)C1] | N-(4-(2- fluorophenyl)- 2-(2-oxa-6- azaspiro[3.3] heptan-6- yl)pyridin-3- yl)-1,3- dimethyl-1H- pyrazole-4- carboxamide | 0.010 g 14% 408.0 (M + 1) | (300 MHz, DMSO- d₆) δ 9.05 (s, 1H), 8.07 (d, J = 4.8 Hz, 1H), 8.01 (s, 1H), 7.38-7.10 (m, 4H), 6.67 (d, J = 4.6 Hz, 1H), 4.66 (s, 4H), 4.17 (s, 4H), 3.75 (s, 3H), 2.11 (s, 3H) |
| 84 | 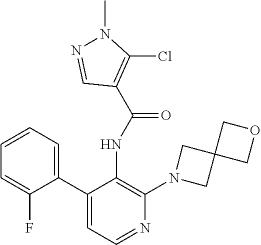 [SMILES: Cn1ncc(C(=O)Nc2c(-c3ccccc3F)ccnc2N2CC3(COC3)C2)c1Cl] | 5-chloro-N- (4-(2- fluorophenyl)- 2-(2-oxa-6- azaspiro[3.3] heptan-6- yl)pyridin-3- yl)-1-methyl- 1H-pyrazole- 4- carboxamide | 0.020 g 27% 428.2 (M + 1) | (300 MHz, DMSO- d₆) δ 9.41 (s, 1H), 8.09 (d, J = 5.2 Hz, 1H), 7.96 (s, 1H), 7.41-7.33 (m, 1H), 7.32-7.13 (m, 3H), 6.72 (dd, J = 5.2, 1.0 Hz, 1H), 4.66 (s, 4H), 4.22 (s, 4H), 3.77 (s, 3H) |

### Example 85

### Synthesis of N-(2-(3-oxa-8-azabicyclo[3.2.1]octan-8-yl)-4-(2-fluorophenyl)pyridin-3-yl)-2-methoxypyrimidine-5-carboxamide

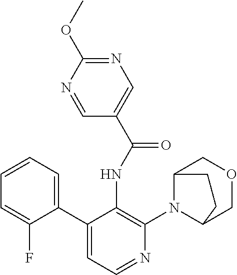 [SMILES: COc1ncc(C(=O)Nc2c(-c3ccccc3F)ccnc2N2C3CCC2COC3)cn1]

### Step 1. Preparation of 8-(4-(2-fluorophenyl)-3-nitropyridin-2-yl)-3-oxa-8-azabicyclo[3.2.1]octane

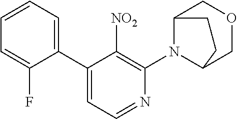 [SMILES: O=[N+]([O-])c1c(-c2ccccc2F)ccnc1N1C2CCC1COC2]

To a mixture of 2-chloro-4-(2-fluorophenyl)-3-nitropyridine (1.50 g, 5.94 mmol) in N-methyl-2-pyrrolidone (30 mL) was added N,N-diisopropylethylamine (5.3 mL, 30 mmol) and 3-oxa-8-azabicyclo[3.2.1]octane hydrochloride (1.78 g, 11.9 mmol). The reaction mixture was stirred at 50° C. for 3 days. After cooling to ambient temperature, the mixture diluted in saturated ammonium chloride (150 mL) and extracted with ethyl acetate (3×150 mL). The combined organic phase was washed with saturated ammonium chloride (3×100 mL), dried over anhydrous magnesium sulfate, filtered and concentrated in vacuo. The residue was purified by column chromatography, eluting with a gradient of 0 to 30% ethyl acetate in heptane, to afford the title compound as a colorless solid (1.95 g, 100% yield): MS (ES+) m/z 330.2 (M+1).

### Step 2. Preparation of 2-(3-oxa-8-azabicyclo[3.2.1]octan-8-yl)-4-(2-fluorophenyl)pyridin-3-amine

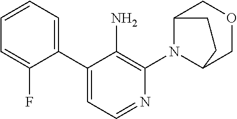 [SMILES: Nc1c(-c2ccccc2F)ccnc1N1C2CCC1COC2]

To a mixture of 8-(4-(2-fluorophenyl)-3-nitropyridin-2-yl)-3-oxa-8-azabicyclo[3.2.1]octane (1.95 g, 5.92 mmol) in methanol (10 mL) and ethyl acetate (10 mL) was added ammonium formate (14.94 g, 236 mmol) and 10% palladium on carbon (0.400 g). The reaction mixture was stirred at 65 degrees for 0.5 h. After cooling to ambient temperature, the mixture was diluted in ethyl acetate (300 mL), washed with saturated sodium bicarbonate (2×100 mL), water (100 mL), dried over anhydrous magnesium sulfate, filtered and concentrated in vacuo. The residue was purified by column chromatography, eluting with a gradient of 0 to 70% ethyl acetate in heptane, to afford the title compound as a pink solid (0.466 g, 26% yield): MS (ES+) m/z 300.2 (M+1).

### Step 3. Preparation of N-(2-(3-oxa-8-azabicyclo[3.2.1]octan-8-yl)-4-(2-fluorophenyl)pyridin-3-yl)-2-methoxypyrimidine-5-carboxamide

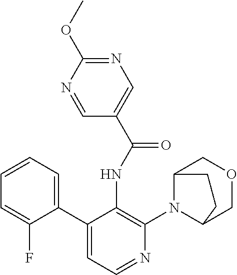 [SMILES: COc1ncc(C(=O)Nc2c(-c3ccccc3F)ccnc2N2C3CCC2COC3)cn1]

To a mixture of 2-(3-oxa-8-azabicyclo[3.2.1]octan-8-yl)-4-(2-fluorophenyl)pyridin-3-amine (0.050 g, 0.17 mmol) in anhydrous tetrahydrofuran (1.7 mL) was added N,N-diisopropylethylamine (0.29 mL, 1.7 mmol), 2-chloro-1-methylpyridinium iodide (0.128 g, 0.50 mmol), 2-methoxypyrimidine-5-carboxylic acid (0.028 g, 0.18 mmol). The reaction mixture was stirred at 65° C. for 4 h. After cooling to ambient temperature, the mixture was diluted in ethyl acetate (100 mL) and washed with saturated ammonium chloride (2×35 mL), dried over anhydrous magnesium sulfate, filtered and concentrated in vacuo. The residue was purified by column chromatography, eluting with a gradient of 30 to 100% ethyl acetate in heptane, to afford the title compound as a colorless solid (0.025 g, 34% yield): ¹H NMR (300 MHz, DMSO-d₆) δ 10.06 (s, 1H), 8.83 (s, 2H), 8.21 (d, J=5.0 Hz, 1H), 7.39-7.31 (m, 2H), 7.27-7.15 (m, 2H), 6.90 (dd, J=5.0, 0.9 Hz, 1H), 4.30 (s, 2H), 3.96 (s, 3H), 3.66 (d, J=10.4 Hz, 2H), 3.49 (d, J=10.6 Hz, 2H), 1.90-1.82 (m, 4H); MS (ES+) m/z 436.2 (M+1).

### Example 86

### Synthesis of N-(4-(2,5-difluorophenyl)-2-morpholinopyridin-3-yl)-4-isopropylbenzamide

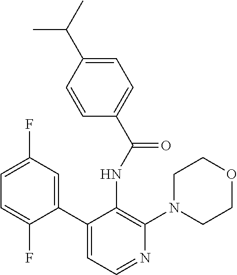 [SMILES: CC(C)c1ccc(C(=O)Nc2c(-c3cc(F)ccc3F)ccnc2N2CCOCC2)cc1]

### Step 1. Preparation of 4-(4-(2,5-difluorophenyl)-3-nitropyridin-2-yl)morpholine

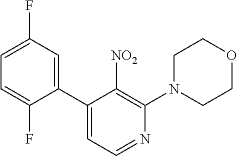 [SMILES: O=[N+]([O-])c1c(-c2cc(F)ccc2F)ccnc1N1CCOCC1]

To a mixture of 2-chloro-4-(2,5-difluorophenyl)-3-nitro-pyridine (3.00 g, 11.1 mmol) in N-methyl-2-pyrrolidone (55 mL) was added N,N-diisopropylethylamine (9.9 mL, 55 mmol) morpholine (1.45 mL, 16.6 mmol) at 0 0° C. The reaction mixture was stirred at ambient temperature for 18 h. The mixture was diluted in saturated ammonium chloride (200 mL) and extracted with ethyl acetate (3×200 mL). The combined organic phase was washed with saturated water (4×200 mL), dried over anhydrous magnesium sulfate, filtered and concentrated in vacuo. The residue was purified by column chromatography, eluting with a gradient of 5 to 45% ethyl acetate in heptane, to afford the title compound as an orange oil (2.08 g, 58% yield): ¹H NMR (300 MHz, DMSO-d₆) δ 8.33 (d, J=4.9 Hz, 1H), 7.12-7.07 (m, 2H), 6.97-6.91 (m, 1H), 6.73 (dd, J=4.9, 0.5 Hz, 1H), 3.79-3.75 (m, 4H), 3.42-3.39 (m, 4H); MS (ES+) m/z 322.2 (M+1).

### Step 2. Preparation of 4-(4-(2,5-difluorophenyl)-3-nitropyridin-2-yl)morpholine

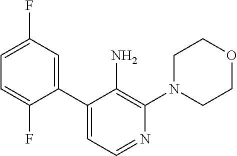 [SMILES: Nc1c(-c2cc(F)ccc2F)ccnc1N1CCOCC1]

To a mixture of 4-(4-(2,5-difluorophenyl)-3-nitropyridin-2-yl)morpholine (2.08 g, 6.47 mmol) in methanol (11 mL) and ethyl acetate (11 mL) was added ammonium formate (8.17 g, 130 mmol) and 10% palladium on carbon (0.207 g). The reaction mixture was stirred at 65 degrees for 4 h. After cooling to ambient temperature, the mixture was diluted in ethyl acetate (300 mL) and filtered through a bed of diatomaceous earth (i.e., Celite®). The filtrate was washed with water (3×100 mL), brine (100 mL), dried over anhydrous magnesium sulfate, filtered and concentrated in vacuo. The residue was purified by column chromatography, eluting with a gradient of 0 to 60% ethyl acetate in heptane, to afford the title compound as a red oil (1.0871 g, 58% yield): MS (ES+) m/z 292.2 (M+1).

### Step 3. Preparation of N-(4-(2,5-difluorophenyl)-2-morpholinopyridin-3-yl)-4-isopropylbenzamide

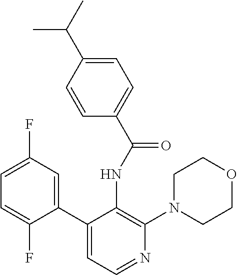 [SMILES: CC(C)c1ccc(C(=O)Nc2c(-c3cc(F)ccc3F)ccnc2N2CCOCC2)cc1]

To a mixture of 4-(4-(2,5-difluorophenyl)-3-nitropyridin-2-yl)morpholine (0.050 g, 0.17 mmol) in anhydrous tetrahydrofuran (1.7 mL) was added N,N-diisopropylethylamine (0.30 mL, 1.7 mmol), 2-chloro-1-methylpyridinium iodide (0.218 g, 0.857 mmol), 4-isopropylbenzoic acid (0.031 g, 0.189 mmol). The reaction mixture was stirred at 65° C. for 4 h. After cooling to ambient temperature, to the reaction mixture was added N,N-diisopropylethylamine (0.15 mL, 0.85 mmol), 2-chloro-1-methylpyridinium iodide (0.145 g, 0.571 mmol), 4-isopropylbenzoic acid (0.031 g, 0.189 mmol) and the reaction mixture was heated to 65° C. for 24 h. After cooling to ambient temperature, to the reaction mixture was added N,N-diisopropylethylamine (0.15 mL, 0.85 mmol), 2-chloro-1-methylpyridinium iodide (0.145 g, 0.571 mmol), 4-isopropylbenzoic acid (0.031 g, 0.189 mmol) and the reaction mixture was heated to 65° C. for 24 h. The reaction mixture was cooled to ambient temperature, diluted in saturated ammonium chloride (50 mL) and extracted with ethyl acetate (3×75 mL). The combined organic phase was washed with saturated ammonium chloride (2×50 mL), dried over anhydrous magnesium sulfate, filtered and concentrated in vacuo. The residue was purified by column chromatography, eluting with a gradient of 0 to 100% ethyl acetate in heptane, to afford the title compound as a colorless solid (0.048 g, 64% yield): ¹H NMR (300 MHz, DMSO-d₆) δ 59.76 (s, 1H), 8.28 (d, J=5.0 Hz, 1H), 7.63 (d, J=8.2 Hz, 2H), 7.32-7.15 (m, 5H), 7.02 (dd, J=5.0, 1.1 Hz, 11H), 3.64 (t, J=4.6 Hz, 4H), 3.22 (t, J=4.5 Hz, 4H), 2.92 (sept, J=6.9 Hz, 1H), 1.20 (d, J=6.9 Hz, 6H); MS (ES+) m/z 438.1 (M+1).

### Example 87-90

In a similar manner as described in EXAMPLE 86, utilizing the appropriately substituted starting materials and intermediates, the following compounds were prepared:

| | | | Amount (g) | |
|---|---|---|---|---|
| Example | | | Yield % | |
| No. | Structure | Name | MS (ES+) m/z | ¹H NMR |
| 87 | 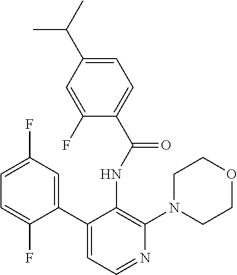 [SMILES: CC(C)c1ccc(C(=O)Nc2c(-c3cc(F)ccc3F)ccnc2N2CCOCC2)c(F)c1] | N-(4-(2,5- difluorophenyl)- 2- morpholinopyridin- 3-yl)-2- fluoro-4- isopropylbenzamide | 0.027 g 35% 456.2 (M + 1) | (300 MHz, DMSO- d₆) δ 9.76 (s, 1H), 8.29 (d, J = 5.0 Hz, 1H), 7.39-7.24 (m, 2H), 7.22-7.09 (m, 4H), 7.03 (dd, J = 5.0, 1.1 Hz, 1H), 3.70 (t, J = 4.6 Hz, 4H), 3.24 (t, J = 4.4 Hz, 4H), 2.92 (sept, J = 6.9 Hz, 1H), 1.18 (d, J = 6.9 Hz, 6H) |
| 88 | 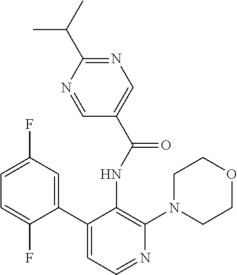 [SMILES: CC(C)c1ncc(C(=O)Nc2c(-c3cc(F)ccc3F)ccnc2N2CCOCC2)cn1] | N-(4-(2,5- difluorophenyl)-2- morpholinopyridin- 3-yl)-2- isopropylpyrimidin- 5-carboxamide | 0.038 g 50% 440.2 (M + 1) | (300 MHz, DMSO- d₆) δ 10.24 (s, 1H), 8.92 (s, 2H), 8.32 (d, J = 5.0 Hz, 1H), 7.36-7.16 (m, 3H), 7.05 (dd, J = 5.0, 0.9 Hz, 1H), 3.66 (t, J = 4.6 Hz, 4H), 3.23-3.13 (m, 5H), 1.27 (d, J = 6.9 Hz, 6H) |
| 89 | 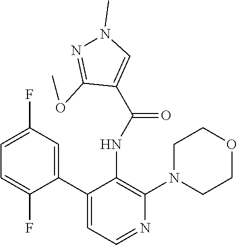 [SMILES: COc1nn(C)cc1C(=O)Nc1c(-c2cc(F)ccc2F)ccnc1N1CCOCC1] | N-(4-(2,5- difluorophenyl)-2- morpholinopyridin- 3-yl)-3- methoxy-1- methyl-1H- pyrazole-4- carboxamide | 0.035 g 47% 430.2 (M + 1) | (300 MHz, DMSO- d₆) δ 8.45 (s, 1H), 8.25 (d, J = 5.0 Hz, 1H), 7.91 (s, 1H), 7.33-7.15 (m, 3H), 7.03 (dd, J = 5.0, 1.2 Hz, 1H), 3.96 (s, 3H), 3.70-3.66 (m, 7H), 3.11 (t, J = 4.6 Hz, 4H) |
| 90 | 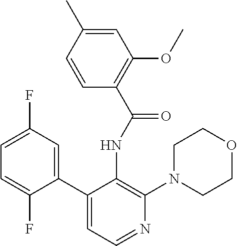 [SMILES: COc1cc(C)ccc1C(=O)Nc1c(-c2cc(F)ccc2F)ccnc1N1CCOCC1] | N-(4-(2,5- difluorophenyl)-2- morpholinopyridin- 3-yl)-2- methoxy-4- methylbenzamide | 0.065 g 57% 440.2 (M + 1) | (300 MHz, DMSO- d₆) δ 9.54 (s, 1H), 8.27 (d, J = 5.0 Hz, 1H), 7.33-7.17 (m, 4H), 7.03 (dd, J = 4.8, 0.2 Hz, 1H), 6.98 (s, 1H), 6.78 (d, J = 7.9 Hz, 1H), 3.94-3.87 (m, 3H), 3.67 (t, J = 4.1 Hz, 4H), 3.20 (t, J = 3.7 Hz, 4H), 2.32 (s, 3H) |

### Example 91

### Synthesis of N-(2-(3-oxa-6-azabicyclo[3.1.1]heptan-6-yl)-4-phenylpyridin-3-yl)-2-isopropylpyrimidine-5-carboxamide

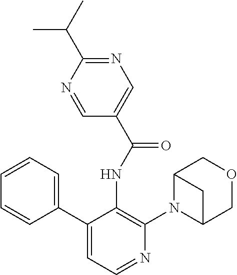 [SMILES: CC(C)c1ncc(C(=O)Nc2c(-c3ccccc3)ccnc2N2C3COCC2C3)cn1]

### Step 1. Preparation of 6-(3-nitro-4-phenylpyridin-2-yl)-3-oxa-6-azabicyclo[3.1.1]heptane

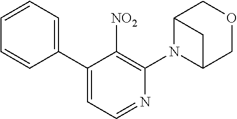 [SMILES: O=[N+]([O-])c1c(-c2ccccc2)ccnc1N1C2COCC1C2]

To a mixture of 2-chloro-3-nitro-4-phenylpyridine (0.610 g, 2.60 mmol) in N-methyl-2-pyrrolidone (8.7 mL) was added N,N-diisopropylethylamine (1.51 g, 8.66 mmol) and 3-oxa-6-azabicyclo[3.1.1]heptane 4-methylbenzene-1-sulfonate (0.235 g, 2.6 mmol). The reaction mixture was stirred at 60° C. for 24 h. After cooling to ambient temperature, the mixture diluted in ethyl acetate (150 mL) and washed with saturated ammonium chloride (5×50 mL), dried over anhydrous magnesium sulfate, filtered and concentrated in vacuo. The residue was purified by column chromatography, eluting with a gradient of 0 to 50% ethyl acetate in heptane, to afford the title compound as a yellow oil (0.193 g, 75% yield): MS (ES+) m/z 298.2 (M+1).

### Step 2. Preparation of 2-(3-oxa-6-azabicyclo[3.1.1]heptan-6-yl)-4-phenylpyridin-3-amine

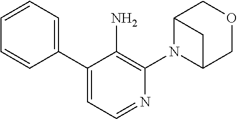 [SMILES: Nc1c(-c2ccccc2)ccnc1N1C2COCC1C2]

To a mixture of 6-(3-nitro-4-phenylpyridin-2-yl)-3-oxa-6-azabicyclo[3.1.1]heptane (0.193 g, 0.650 mmol) in methanol (2.2 mL) and ethyl acetate (2.2 mL) was added ammonium formate (1.016 g, 16.11 mmol) and 10% palladium on carbon (0.050 g). The reaction mixture was stirred at 65 degrees for 0.5 h. After cooling to ambient temperature, the mixture was diluted in ethyl acetate (200 mL), filtered through a bed of diatomaceous earth (i.e., Celite®), and concentrated in vacuo.

The residue was purified by column chromatography, eluting with a gradient of 0 to 70% ethyl acetate in heptane, to afford the title compound as a red oil (0.167 g, 96% yield): MS (ES+) m/z 268.2 (M+1).

### Step 3. N-(2-(3-oxa-6-azabicyclo[3.1.1]heptan-6-yl)-4-phenylpyridin-3-yl)-2-isopropylpyrimidine-5-carboxamide

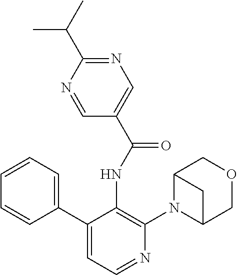 [SMILES: CC(C)c1ncc(C(=O)Nc2c(-c3ccccc3)ccnc2N2C3COCC2C3)cn1]

To a mixture of 2-(3-oxa-6-azabicyclo[3.1.1]heptan-6-yl)-4-phenylpyridin-3-amine (0.055 g, 0.20 mmol) in anhydrous tetrahydrofuran (2.0 mL) was added N,N-diisopropylethylamine (0.36 mL, 2.0 mmol), 2-chloro-1-methylpyridinium iodide (0.157 g, 0.614 mmol), 2-isopropylpyrimidine-5-carboxylic acid (0.037 g, 0.23 mmol). The reaction mixture was stirred at 65° C. for 2.5 h. After cooling to ambient temperature, the mixture was diluted in ethyl acetate (100 mL), washed with saturated ammonium chloride (2×30 mL), dried over anhydrous magnesium sulfate, filtered and concentrated in vacuo. The residue was purified by column chromatography, eluting with a gradient of 30 to 80% ethyl acetate in heptane, to afford the title compound as a colorless solid (0.063 g, 74% yield): ¹H NMR (300 MHz, DMSO-d₆) δ 10.07 (s, 1H), 8.92 (s, 2H), 8.18 (d, J=5.0 Hz, 1H), 7.43-7.29 (m, 5H), 6.77 (d, J=5.0 Hz, 1H), 4.37-4.18 (m, 4H), 3.63 (m, 2H), 3.16 (sept, J=6.9 Hz, 1H), 2.62 (dd, J=6.7 Hz, 1H), 1.73 (d, J=8.0 Hz, 1H), 1.26 (d, J=6.9 Hz, 6H); MS (ES+) m/z 416.2 (M+1).

### Example 92

### Synthesis of N-(2-(3,3-difluoropyrrolidin-1-yl)-4-(2-fluorophenyl)pyridin-3-yl)-4-methoxypiperidine-1-carboxamide

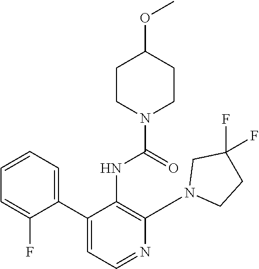 [SMILES: COC1CCN(C(=O)Nc2c(-c3ccccc3F)ccnc2N2CCC(F)(F)C2)CC1]

### Step 1. Preparation of 2-chloro-4-(2-fluorophenyl)-3-nitropyridine

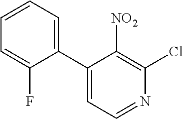 [SMILES: O=[N+]([O-])c1c(-c2ccccc2F)ccnc1Cl]

A mixture of 2,4-dichloro-3-nitropyridine (5.0 g, 26 mmol), 1,4-dioxane (50 mL), and water (17 mL) was sparged with nitrogen for 10 min. To the mixture was added 2-fluorophenylboronic acid (3.6 g, 26 mmol), potassium carbonate (5.4 g, 39 mmol), and [1,1′-bis(diphenylphosphino)ferrocene]dichloropalladium(II) complex with dichloromethane (2.2 g, 2.6 mmol), and sparged with nitrogen for 2 min. The reaction mixture was stirred at 60° C. for 4 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 0-30% ethyl acetate in heptane, afforded the title compound as a colorless solid (4.0 g, 61% yield).

### Step 2. Preparation of 4-(2-fluorophenyl)-2-(3,3-difluoropyrrolidin-1-yl)-3-nitropyridine

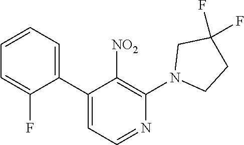 [SMILES: O=[N+]([O-])c1c(-c2ccccc2F)ccnc1N1CCC(F)(F)C1]

To a mixture of 2-chloro-4-(2-fluorophenyl)-3-nitropyridine (2.0 g, 7.9 mmol), anhydrous potassium carbonate (3.3 g, 24 mmol), and 3,3-difluoropyrrolidine hydrochloride (1.5 g, 10 mmol) was added N,N-dimethylformamide (26 mL). The reaction mixture was stirred at ambient temperature for 24 h. The reaction mixture was diluted with ethyl acetate (200 mL). The organic layer was washed with saturated ammonium chloride (2×50 mL). The organic solution was dried over anhydrous magnesium sulfate, filtered, and concentrated in vacuo. Purification of the residue by column chromatography, eluting with 5-35% ethyl acetate in heptane, afforded the title compound as a yellow oil (2.5 g, 98% yield): MS (ES+) m/z 324.2 (M+1).

### Step 3. Preparation of 2-(3,3-difluoropyrrolidin-1-yl)-4-(2-fluorophenyl)pyridin-3-amine

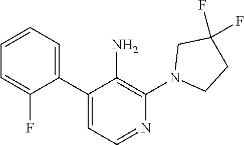 [SMILES: Nc1c(-c2ccccc2F)ccnc1N1CCC(F)(F)C1]

To 4-(2-fluorophenyl)-2-(3,3-difluoropyrrolidin-1-yl)-3-nitropyridine (2.5 g, 7.8 mmol) was added anhydrous methanol (13 mL), ethyl acetate (13 mL), and 10% palladium on carbon (0.83 g). The reaction vessel was sealed and the reaction mixture was sparged with hydrogen gas for 5 min. The reaction mixture was stirred under a hydrogen atmosphere for 24 h. The reaction mixture was filtered through diatomaceous earth (i.e., Celite®), washed with ethyl acetate (5×20 mL) and concentrated in vacuo. Purification of the residue by column chromatography, eluting with 5-35% ethyl acetate in heptane, afforded the title compound as a clear colorless oil (1.6 g, 72% yield): MS (ES+) m/z 294.2 (M+1).

### Step 4. Preparation of N-(2-(3,3-difluoropyrrolidin-1-yl)-4-(2-fluorophenyl)pyridin-3-yl)-4-methoxypiperidine-1-carboxamide

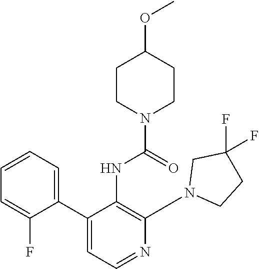 [SMILES: COC1CCN(C(=O)Nc2c(-c3ccccc3F)ccnc2N2CCC(F)(F)C2)CC1]

To 2-(3,3-difluoropyrrolidin-1-yl)-4-(2-fluorophenyl)pyridin-3-amine (0.10 g, 0.34 mmol) was added anhydrous tetrahydrofuran (1.1 mL) and the mixture was cooled in an ice-water bath. To the mixture was added triphosgene (0.067 g, 0.23 mmol). The solution was stirred at 0° C. for 2.5 h before 4-methoxypiperidine (0.24 g, 2.0 mmol), anhydrous tetrahydrofuran (1.1 mL), and N-ethyl-N-isopropylpropan-2-amine (0.44 g, 3.4 mmol) were added. The reaction mixture was warmed to ambient temperature and stirred for 2 h. The reaction mixture was diluted with ethyl acetate (150 mL), washed with saturated ammonium chloride (2×50 mL), dried over anhydrous magnesium sulfate, filtered, and concentrated in vacuo. Purification of the residue by column chromatography, eluting with a gradient of 10 to 100% of ethyl acetate in heptane, to provide the title compound as a colorless solid (0.050 g, 32% yield): ¹H-NMR (500 MHz; DMSO-d₆) δ 8.09 (d, J=4.9 Hz, 1H), 7.90 (s, 1H), 7.42-7.38 (m, 1H), 7.29-7.18 (m, 3H), 6.69-6.68 (m, 1H), 3.97-3.83 (m, 2H), 3.81-3.71 (m, 2H), 3.53-3.48 (m, 2H), 3.24-3.18 (m, 4H), 2.91-2.83 (m, 2H), 2.48-2.40 (m, 2H), 1.54-1.50 (m, 2H), 1.03-0.96 (m, 2H); MS (ES+) m/z 435.2 (M+1).

### Example 93

### Synthesis of N-[2-(3,3-difluoropyrrolidin-1-yl)-4-(2-fluorophenyl)-3-pyridyl]-7-methoxy-2-azaspiro[3.5]nonane-2-carboxamide

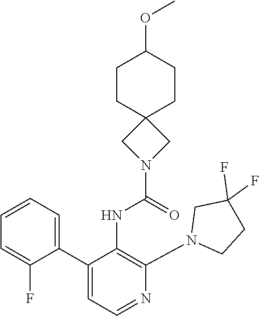 [SMILES: COC1CCC2(CC1)CN(C(=O)Nc1c(-c3ccccc3F)ccnc1N1CCC(F)(F)C1)C2]

### Step 1. Preparation of N-[2-(3,3-difluoropyrrolidin-1-yl)-4-(2-fluorophenyl)-3-pyridyl]-7-methoxy-2-azaspiro[3.5]nonane-2-carboxamide

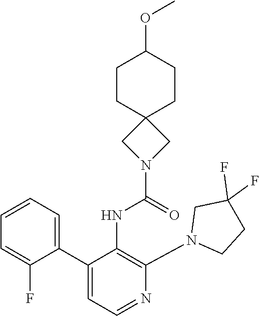 [SMILES: COC1CCC2(CC1)CN(C(=O)Nc1c(-c3ccccc3F)ccnc1N1CCC(F)(F)C1)C2]

To 2-(3,3-difluoropyrrolidin-1-yl)-4-(2-fluorophenyl)pyridin-3-amine (0.11 g, 0.38 mmol) was added anhydrous tetrahydrofuran (3.8 mL) and the mixture was cooled in an ice-water bath. To the mixture was added triphosgene (0.057 g, 0.19 mmol). The solution was stirred at 0° C. for 2.5 h before 7-methoxy-2-azaspiro[3.5]nonane (0.12 g, 0.76 mmol), anhydrous tetrahydrofuran (1.0 mL), and N-ethyl-N-isopropylpropan-2-amine (0.49 g, 3.8 mmol) were added. The reaction was allowed to warm to ambient temperature and stir for 2 h. The reaction mixture was diluted with ethyl acetate (150 mL), washed with saturated ammonium chloride (2×50 mL), dried over anhydrous magnesium sulfate, filtered, and concentrated in vacuo. Purification by preparative HPLC, eluting with a gradient of 10 to 80% of acetonitrile in water containing 0.5% formic acid, provided the title compound as a colorless solid (0.053 g, 28% yield): ¹H-NMR (300 MHz; DMSO-d₆) δ 5 8.09 (d, J=4.9 Hz, 1H), 7.79 (s, 1H), 7.47-7.40 (m, 1H), 7.32-7.25 (m, 3H), 6.69 (dd, J=5.0, 0.8 Hz, 1H), 3.96-3.87 (m, 2H), 3.75 (t, J=7.3 Hz, 2H), 3.24-3.21 (m, 4H), 3.21-3.19 (m, 3H), 3.09-3.07 (m, 1H), 2.49-2.39 (m, 2H), 1.68-1.61 (m, 2H), 1.56-1.49 (m, 2H), 1.32-1.23 (m, 2H), 1.21-1.13 (m, 2H); MS (ES+) m/z 475.2 (M+1).

### Example 94

### Synthesis of N-[2-(3,3-difluoropyrrolidin-1-yl)-4-(2-fluorophenyl)-3-pyridyl]-6-methoxy-2-azaspiro[3.3]heptane-2-carboxamide

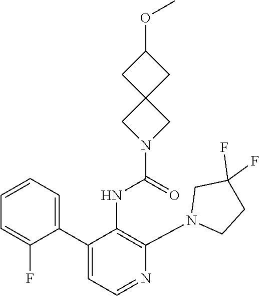 [SMILES: COC1CC2(C1)CN(C(=O)Nc1c(-c3ccccc3F)ccnc1N1CCC(F)(F)C1)C2]

### Step 1. Preparation of N-[2-(3,3-difluoropyrrolidin-1-yl)-4-(2-fluorophenyl)-3-pyridyl]-6-methoxy-2-azaspiro[3.3]heptane-2-carboxamide

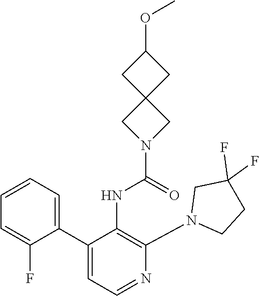 [SMILES: COC1CC2(C1)CN(C(=O)Nc1c(-c3ccccc3F)ccnc1N1CCC(F)(F)C1)C2]

To 2-(3,3-difluoropyrrolidin-1-yl)-4-(2-fluorophenyl)pyridin-3-amine (0.11 g, 0.38 mmol) was added anhydrous tetrahydrofuran (3.6 mL) and cooled in an ice-water bath.

To the mixture was added triphosgene (0.11 g, 0.36 mmol). The solution was stirred at 0° C. for 18 h before 6-methoxy-2-azaspiro[3.3]heptane hydrochloride (0.12 g, 0.73 mmol), anhydrous tetrahydrofuran (3.0 mL), and N-ethyl-N-isopropylpropan-2-amine (0.47 g, 3.6 mmol) were added. The reaction was allowed to warm to ambient temperature and stir for 2 h. The reaction mixture was diluted with ethyl acetate (100 mL), washed with saturated ammonium chloride (2×50 mL), dried over anhydrous magnesium sulfate, filtered, and concentrated in vacuo. Purification by preparative HPLC, eluting with a gradient of 10 to 80% of acetonitrile in water containing 0.5% formic acid, provided the title compound as a colorless solid (0.083 g, 50% yield): ¹H-NMR (300 MHz; DMSO-d₆) δ 8.09 (d, J=4.9 Hz, 1H), 7.80 (s, 1H), 7.47-7.41 (m, 1H), 7.32-7.24 (m, 3H), 6.70 (dd, J=5.0, 0.8 Hz, 1H), 3.90 (t, J=13.6 Hz, 2H), 3.74 (t, J=7.2 Hz, 2H), 3.64 (t, J=6.8 Hz, 1H), 3.52 (s, 2H), 3.46 (s, 2H), 3.07 (s, 3H), 2.43 (dt, J=14.2, 7.1 Hz, 2H), 2.25 (ddd, J=9.8, 6.8, 2.9 Hz, 2H), 1.88-1.81 (m, 2H); MS (ES+) m/z 447.2 (M+1).

### Example 95

### Synthesis of N-[2-(3,3-difluoropyrrolidin-1-yl)-4-(2-fluorophenyl)-3-pyridyl]-7-oxa-2-azaspiro[3.5]nonane-2-carboxamide

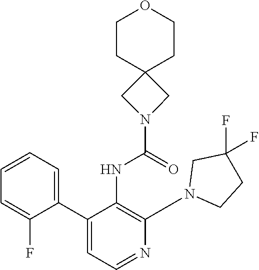 [SMILES: O=C(Nc1c(-c2ccccc2F)ccnc1N1CCC(F)(F)C1)N1CC2(CCOCC2)C1]

To 2-(3,3-difluoropyrrolidin-1-yl)-4-(2-fluorophenyl)pyridin-3-amine (0.12 g, 0.39 mmol) was added anhydrous tetrahydrofuran (3.9 mL) and cooled in an ice-water bath. To the mixture was added triphosgene (0.092 g, 0.31 mmol). The solution was stirred at 0° C. for 2.5 h before 7-oxa-2-azaspiro[3.5]nonane hydrochloride (0.13 g, 0.78 mmol), anhydrous tetrahydrofuran (1.0 mL), anhydrous N,N-dimethylformamide (0.5 mL), and N-ethyl-N-isopropylpropan-2-amine (0.51 g, 3.9 mmol) were added. The reaction was allowed to warm to ambient temperature and stir for 18 h. The reaction mixture was diluted with ethyl acetate (150 mL), washed with saturated ammonium chloride (2×50 mL), dried over anhydrous magnesium sulfate, filtered, and concentrated in vacuo. Purification by column chromatography, eluting with 35-100% ethyl acetate in heptane, provided the title compound as a colorless solid (0.083 g, 50% yield): ¹H-NMR (300 MHz; DMSO-d₆) δ 8.10-8.09 (m, 1H), 7.86-7.82 (m, 1H), 7.47-7.39 (m, 1H), 7.31-7.23 (m, 3H), 6.70 (dd, J=5.0, 0.8 Hz, 1H), 3.96-3.83 (m, 2H), 3.76 (quintet, J=6.8 Hz, 2H), 3.43-3.39 (m, 4H), 3.34-3.28 (m, 4H), 2.49-2.37 (m, 2H), 1.45 (t, J=5.0 Hz, 4H); MS (ES+) m/z 447.2 (M+1).

### Example 96

### Synthesis of tert-butyl 2-[[2-(3,3-difluoropyrrolidin-1-yl)-4-(2-fluorophenyl)-3-pyridyl]carbamoyl]-2, 7-diazaspiro[3.5]nonane-7-carboxylate

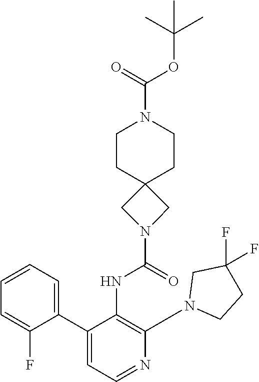 [SMILES: CC(C)(C)OC(=O)N1CCC2(CC1)CN(C(=O)Nc1c(-c3ccccc3F)ccnc1N1CCC(F)(F)C1)C2]

To 2-(3,3-difluoropyrrolidin-1-yl)-4-(2-fluorophenyl)pyridin-3-amine (0.12 g, 0.39 mmol) was added anhydrous tetrahydrofuran (4.1 mL) and cooled in an ice-water bath. To the mixture was added triphosgene (0.096 g, 0.32 mmol). The solution was stirred at 0° C. for 2.5 h before tert-butyl 2,7-diazaspiro[3.5]nonane-7-carboxylate hydrochloride (0.22 g, 0.82 mmol), anhydrous tetrahydrofuran (1.0 mL), anhydrous N,N-dimethylformamide (0.5 mL), and N-ethyl-N-isopropylpropan-2-amine (0.53 g, 4.1 mmol) were added. The reaction was allowed to warm to ambient temperature and stir for 30 min. The reaction mixture was diluted with ethyl acetate (150 mL), washed with saturated ammonium chloride (2×50 mL), dried over anhydrous magnesium sulfate, filtered, and concentrated in vacuo. Purification by column chromatography, eluting with 20-100% ethyl acetate in heptane, provided the title compound as a colorless solid (0.13 g, 52% yield): ¹H-NMR (300 MHz; DMSO-d₆) δ 8.10-8.09 (m, 1H), 7.84 (s, 1H), 7.45 (ddd, J=8.4, 5.4, 3.3 Hz, 1H), 7.30-7.26 (m, 3H), 6.70 (dd, J=5.0, 0.8 Hz, 1H), 3.96-3.86 (m, 2H), 3.78-3.73 (m, 2H), 3.29 (s, 4H), 3.18-3.16 (m, 4H), 2.43 (dd, J=14.2, 7.1 Hz, 2H), 1.39 (d, J=4.0 Hz, 13H); MS (ES+) m/z 546.2 (M+1).

### Example 97

### Synthesis of 7-acetyl-N-[2-(3,3-difluoropyrrolidin-1-yl)-4-(2-fluorophenyl)-3-pyridyl]-2,7-diazaspiro[3.5]nonane-2-carboxamide

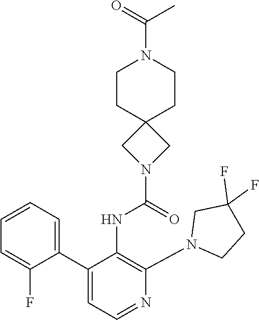 [SMILES: CC(=O)N1CCC2(CC1)CN(C(=O)Nc1c(-c3ccccc3F)ccnc1N1CCC(F)(F)C1)C2]

### Step 1. Preparation of N-[2-(3,3-difluoropyrrolidin-1-yl)-4-(2-fluorophenyl)-3-pyridyl]-2,7-diazaspiro[3.5]nonane-2-carboxamide

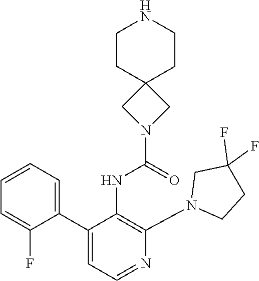 [SMILES: O=C(Nc1c(-c2ccccc2F)ccnc1N1CCC(F)(F)C1)N1CC2(CCNCC2)C1]

To tert-butyl 2-[[2-(3,3-difluoropyrrolidin-1-yl)-4-(2-fluorophenyl)-3-pyridyl]carbamoyl]-2,7-diazaspiro[3.5]nonane-7-carboxylate (0.11 g, 0.20 mmol) was added anhydrous dichloromethane (2.0 mL) and trifluoroacetic acid (2.0 mL) at ambient temperature. The solution was stirred at ambient temperature for 1 h. The reaction mixture was diluted with ethyl acetate (200 mL), washed with saturated sodium bicarbonate (3×50 mL), dried over anhydrous magnesium sulfate, filtered, and concentrated in vacuo. The residue obtained was used in the without further purification (0.090 g, 100% yield): MS (ES+) m/z 446.2 (M+1).

### Step 2. Preparation of 7-acetyl-N-[2-(3,3-difluoropyrrolidin-1-yl)-4-(2-fluorophenyl)-3-pyridyl]-2,7-diazaspiro[3.5]nonane-2-carboxamide

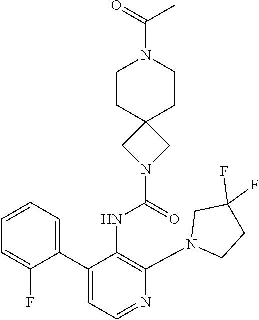 [SMILES: CC(=O)N1CCC2(CC1)CN(C(=O)Nc1c(-c3ccccc3F)ccnc1N1CCC(F)(F)C1)C2]

To N-[2-(3,3-difluoropyrrolidin-1-yl)-4-(2-fluorophenyl)-3-pyridyl]-2,7-diazaspiro[3.5]nonane-2-carboxamide (0.090 g, 0.20 mmol) was added anhydrous dichloromethane (2.0 mL) and cooled to 0° C. in an ice-water bath. To the mixture was added N-ethyl-N-isopropylpropan-2-amine (0.052 g, 0.40 mmol) and acetyl chloride (0.024 g, 0.30 mmol). The reaction mixture was allowed to warm to ambient temperature and stir for 30 min. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with 20-100% ethyl acetate in heptane, then 0-50% methanol in ethyl acetate, provided the title compound as a colorless solid (0.13 g, 52% yield): ¹H-NMR (300 MHz; DMSO-d₆) δ 8.10 (d, J=4.9 Hz, 1H), 7.84 (s, 1H), 7.50-7.42 (m, 1H), 7.33-7.26 (m, 3H), 6.71-6.69 (m, 1H), 3.96-3.86 (m, 2H), 3.78-3.72 (m, 2H), 3.34-3.22 (m, 8H), 2.47-2.37 (m, 2H), 1.99 (s, 3H), 1.48-1.44 (m, 2H), 1.40-1.33 (m, 2H); MS (ES+) m/z 488.2 (M+1).

### Example 98

### Synthesis of N-[2-(3,3-difluoropyrrolidin-1-yl)-4-(2-fluorophenyl)-3-pyridyl]-2-methoxy-7-azaspiro[3.5]nonane-7-carboxamide

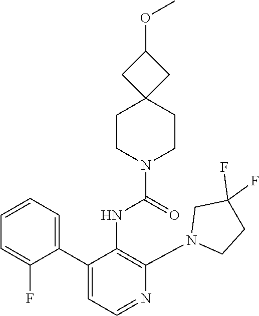 [SMILES: COC1CC2(CCN(C(=O)Nc3c(-c4ccccc4F)ccnc3N3CCC(F)(F)C3)CC2)C1]

To 2-(3,3-difluoropyrrolidin-1-yl)-4-(2-fluorophenyl)pyridin-3-amine (0.070 g, 0.24 mmol) was added anhydrous tetrahydrofuran (2.4 mL) and cooled to 0° C. in an ice-water bath. To the mixture was added triphosgene (0.035 g, 0.12 mmol). The solution was stirred at 0° C. for 2.5 h before 2-methoxy-7-azaspiro[3.5]nonane hydrochloride (0.092 g, 0.48 mmol), anhydrous tetrahydrofuran (1.5 mL) and N-ethyl-N-isopropylpropan-2-amine (0.31 g, 2.4 mmol) were added. The reaction mixture was allowed to warm to ambient temperature and stir for 1 h. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with 20-100% ethyl acetate in heptane, followed by preparative HPLC, eluting with a gradient of 10 to 80% of acetonitrile in water containing 0.5% formic acid, provided the title compound as a colorless solid (0.062 g, 55% yield): ¹H-NMR (300 MHz; DMSO-d₆) δ 8.08 (d, J=5.0 Hz, 1H), 7.86 (s, 1H), 7.43-7.35 (m, 1H), 7.29-7.15 (m, 3H), 6.67 (dd, J=5.0, 0.8 Hz, 1H), 3.94-3.71 (m, 5H), 3.12-3.06 (m, 7H), 2.43 (ddd, J=20.8, 13.6, 6.6 Hz, 2H), 2.06-1.99 (m, 2H), 1.52-1.45 (m, 2H), 1.08 (d, J=11.0 Hz, 4H); MS (ES+) m/z 475.4 (M+1).

### Example 99

### Synthesis of (1R,5S)-N-[2-(3,3-difluoropyrrolidin-1-yl)-4-(2-fluorophenyl)-3-pyridyl]-3-methoxy-8-azabicyclo[3.2.1]octane-8-carboxamide

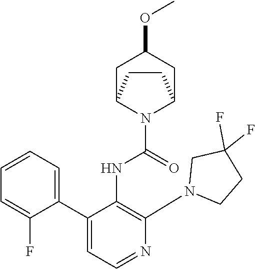 [SMILES: CO[C@@H]1C[C@H]2CC[C@@H](C1)N2C(=O)Nc1c(-c2ccccc2F)ccnc1N1CCC(F)(F)C1]

To 2-(3,3-difluoropyrrolidin-1-yl)-4-(2-fluorophenyl)pyridin-3-amine (0.070 g, 0.24 mmol) was added anhydrous tetrahydrofuran (2.4 mL) and cooled to 0° C. in an ice-water bath. To the mixture was added triphosgene (0.035 g, 0.12 mmol). The solution was stirred at 0° C. for 2.5 h before (1R,5S)-3-methoxy-8-azabicyclo[3.2.1]octane hydrochloride (0.085 g, 0.48 mmol), anhydrous tetrahydrofuran (1.5 mL) and N-ethyl-N-isopropylpropan-2-amine (0.31 g, 2.4 mmol) were added. The reaction mixture was allowed to warm to ambient temperature and stir for 1 h. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with 20-100% ethyl acetate in heptane, followed by preparative HPLC, eluting with a gradient of 10 to 80% of acetonitrile in water containing 0.5% formic acid, provided the title compound as a colorless solid (0.062 g, 55% yield): ¹H-NMR (300 MHz; DMSO-d₆) δ 8.09 (d, J=4.9 Hz, 1H), 7.84 (s, 1H), 7.40-7.32 (m, 1H), 7.30-7.14 (m, 3H), 6.69 (d, J=4.8 Hz, 1H), 4.13-4.09 (m, 2H), 4.00-3.83 (m, 2H), 3.83-3.70 (m, 2H), 3.54-3.43 (m, 1H), 3.16-3.11 (m, 3H), 2.50-2.36 (m, 2H), 1.79-1.70 (m, 2H), 1.62-1.45 (m, 4H), 1.02-0.89 (m, 2H); MS (ES+) m/z 461.2 (M+1).

### Example 100

### Synthesis of N-[2-(3,3-difluoropyrrolidin-1-yl)-4-(2-fluorophenyl)-3-pyridyl]-2-(3,3-dimethylbutanoyl)-2,7-diazaspiro[3.5]nonane-7-carboxamide

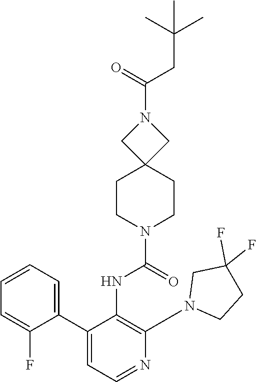 [SMILES: CC(C)(C)CC(=O)N1CC2(CCN(C(=O)Nc3c(-c4ccccc4F)ccnc3N3CCC(F)(F)C3)CC2)C1]

### Step 1. Preparation of tert-butyl 7-[[2-(3,3-difluoropyrrolidin-1-yl)-4-(2-fluorophenyl)-3-pyridyl]carbamoyl]-2,7-diazaspiro[3.5]nonane-2-carboxylate

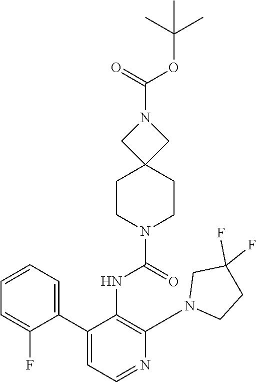 [SMILES: CC(C)(C)OC(=O)N1CC2(CCN(C(=O)Nc3c(-c4ccccc4F)ccnc3N3CCC(F)(F)C3)CC2)C1]

To 2-(3,3-difluoropyrrolidin-1-yl)-4-(2-fluorophenyl)pyridin-3-amine (0.15 g, 0.51 mmol) was added anhydrous tetrahydrofuran (5.1 mL) and cooled to 0° C. in an ice-water bath. To the mixture was added triphosgene (0.12 g, 0.41 mmol). The solution was stirred at 0° C. for 2.5 h before tert-butyl 2,7-diazaspiro[3.5]nonane-2-carboxylate (0.35 g, 1.5 mmol), anhydrous tetrahydrofuran (2.0 mL) and N-ethyl-N-isopropylpropan-2-amine (0.66 g, 5.1 mmol) were added. The reaction mixture was allowed to warm to ambient temperature and stir for 1 h. The reaction mixture was diluted with ethyl acetate (150 mL), washed with saturated ammonium chloride (2×50 mL), dried over anhydrous magnesium sulfate, filtered, and concentrated in vacuo.

Purification by column chromatography, eluting with 20-100% ethyl acetate in heptane, provided the title compound as a colorless solid (0.24 g, 86% yield): ¹H-NMR (300 MHz; DMSO-d₆) δ 8.08 (d, J=4.9 Hz, 1H), 7.95 (s, 1H), 7.44-7.36 (m, 1H), 7.28-7.17 (m, 3H), 6.68 (d, J=4.9 Hz, 1H), 3.94-3.80 (m, 2H), 3.80-3.69 (m, 4H), 3.44-3.42 (m, 2H), 3.21-3.07 (m, 4H), 2.49-2.36 (m, 2H), 1.27-1.16 (m, 4H), 0.94 (s, 9H); MS (ES+) m/z546.4(M+1).

### Step 2. Preparation of N-[2-(3,3-difluoropyrrolidin-1-yl)-4-(2-fluorophenyl)-3-pyridyl]-2,7-diazaspiro[3.5]nonane-7-carboxamide

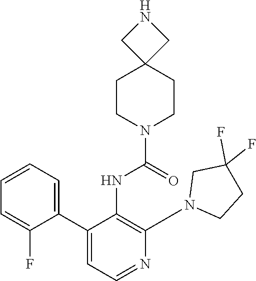 [SMILES: O=C(Nc1c(-c2ccccc2F)ccnc1N1CCC(F)(F)C1)N1CCC2(CC1)CNC2]

To tert-butyl 7-[[2-(3,3-difluoropyrrolidin-1-yl)-4-(2-fluorophenyl)-3-pyridyl]carbamoyl]-2,7-diazaspiro[3.5]nonane-2-carboxylate (0.24 g, 0.44 mmol) was added anhydrous dichloromethane (5.0 mL) and trifluoroacetic acid (4.0 mL). The reaction was stirred at ambient temperature for 3 h. The reaction mixture was diluted with ethyl acetate (250 mL), washed with 50% sodium bicarbonate (4×50 mL), saturated ammonium chloride (50 mL), dried over anhydrous magnesium sulfate, filtered, and concentrated in vacuo. The resulting residue was used as is in the next step (0.20 g, 88% yield): MS (ES+) m/z 446.2 (M+1).

### Step 3. Preparation of N-[2-(3,3-difluoropyrrolidin-1-yl)-4-(2-fluorophenyl)-3-pyridyl]-2-(3,3-dimethylbutanoyl)-2,7-diazaspiro[3.5]nonane-7-carboxamide

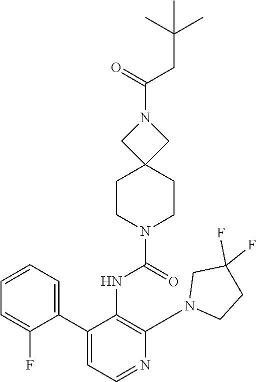 [SMILES: CC(C)(C)CC(=O)N1CC2(CCN(C(=O)Nc3c(-c4ccccc4F)ccnc3N3CCC(F)(F)C3)CC2)C1]

To N-[2-(3,3-difluoropyrrolidin-1-yl)-4-(2-fluorophenyl)-3-pyridyl]-2,7-diazaspiro[3.5]nonane-7-carboxamide (0.040 g, 0.090 mmol) were added anhydrous dichloromethane (1.0 mL) and cooled to −78° C. N-Ethyl-N-isopropylpropan-2-amine (0.035 g, 0.27 mmol) and tert-butylacetyl chloride (0.018 g, 0.13 mmol) were added and the reaction was allowed to warm to ambient temperature and stir for 1 h. The reaction mixture was diluted with ethyl acetate (150 mL), washed with saturated ammonium chloride (2×50 mL), dried over anhydrous magnesium sulfate, filtered, and concentrated in vacuo. Purification by column chromatography, eluting with 20-100% ethyl acetate in heptane and 0 to 50% methanol in ethyl acetate followed by preparative HPLC eluting with a gradient of 10 to 60% of acetonitrile in water containing 0.5% formic acid to provide the title compound as a colorless solid (0.0077 g, 16% yield): ¹H-NMR (300 MHz; DMSO-d₆) δ 8.08 (d, J=4.9 Hz, 1H), 7.95 (s, 1H), 7.44-7.36 (m, 1H), 7.28-7.17 (m, 3H), 6.68 (d, J=4.9 Hz, 1H), 3.96-3.82 (m, 2H), 3.82-3.68 (m, 4H), 3.44-3.42 (m, 2H), 3.21-3.07 (m, 4H), 2.48-2.36 (m, 2H), 1.92-1.88 (m, 2H), 1.27-1.17 (m, 4H), 0.94-0.93 (m, 9H); MS (ES+) m/z 544.2 (M+1).

### Example 101

### Synthesis of tert-butyl 6-[[2-(3,3-difluoropyrrolidin-1-yl)-4-(2-fluorophenyl)-3-pyridyl]carbamoyl]-2,6-diazaspiro[3.3]heptane-2-carboxylate

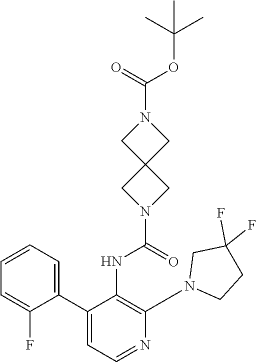 [SMILES: CC(C)(C)OC(=O)N1CC2(CN(C(=O)Nc3c(-c4ccccc4F)ccnc3N3CCC(F)(F)C3)C2)C1]

To 2-(3,3-difluoropyrrolidin-1-yl)-4-(2-fluorophenyl)pyridin-3-amine (0.20 g, 0.70 mmol) was added anhydrous tetrahydrofuran (3.5 mL) and cooled to 0° C. To the mixture was added triphosgene (0.17 g, 0.56 mmol). The solution was stirred at 0° C. for 18 h before tert-butyl 2,6-diazaspiro[3.3]heptane-2-carboxylate hydrochloride (0.49 g, 2.1 mmol), anhydrous tetrahydrofuran (4.0 mL) and N-ethyl-N-isopropylpropan-2-amine (0.90 g, 7.0 mmol) were added. The reaction mixture was allowed to warm to ambient temperature and stir for 2 h. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with 5-100% ethyl acetate in heptane, provided the title compound as a colorless solid (0.33 g, 86% yield): ¹H-NMR (300 MHz; DMSO-d₆) δ 8.09 (d, J=4.9 Hz, 1H), 7.92 (s, 1H), 7.47-7.41 (m, 1H), 7.34-7.29 (m, 1H), 7.27-7.24 (m, 2H), 6.71 (dd, J=5.0, 0.8 Hz, 1H), 3.94-3.79 (m, 6H), 3.76-3.72 (m, 2H), 3.64 (s, 4H), 2.48-2.38 (m, 2H), 1.36 (s, 9H); MS (ES+) m/z 518.2 (M+1).

### Example 102

### Synthesis of N-[2-(3,3-difluoropyrrolidin-1-yl)-4-(2-fluorophenyl)-3-pyridyl]-6-(2,2,2-trifluoroethyl)-2,6-diazaspiro[3.3]heptane-2-carboxamide

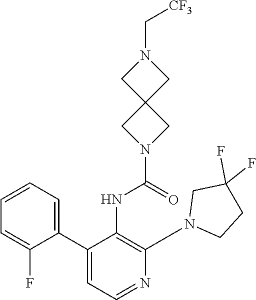 [SMILES: O=C(Nc1c(-c2ccccc2F)ccnc1N1CCC(F)(F)C1)N1CC2(CN(CC(F)(F)F)C2)C1]

To tert-butyl 6-[[2-(3,3-difluoropyrrolidin-1-yl)-4-(2-fluorophenyl)-3-pyridyl]carbamoyl]-2,6-diazaspiro[3.3]heptane-2-carboxylate (0.19 g, 0.34 mmol) was added anhydrous dichloromethane (5.0 mL) and trifluoroacetic acid (4.0 mL) and stirred at ambient temperature for 18 h. The reaction mixture was diluted with ethyl acetate (200 mL), washed with 1:1 mixture of 1 M sodium hydroxide: brine (2×50 mL), dried over anhydrous magnesium sulfate, filtered, and concentrated in vacuo. The residue was dissolved in anhydrous tetrahydrofuran (0.78 mL) and to the solution was added N-ethyl-N-isopropylpropan-2-amine (0.22 g, 1.7 mmol) and 2,2,2-trifluoroethyl trifluoromethanesulfonate (0.16 g, 0.68 mmol). The vial was sealed and heated to 50° C. The reaction mixture was cooled to ambient temperature, diluted with ethyl acetate (150 mL), washed with brine (50 mL), dried over anhydrous magnesium sulfate, filtered, and concentrated in vacuo. Purification by column chromatography, eluting with 40-100% ethyl acetate in heptane, followed by preparative HPLC, eluting with a gradient of 10 to 40% of acetonitrile in water containing 0.5% formic acid, to provide the title compound as a colorless solid (0.023 g, 13% yield): ¹H-NMR (300 MHz; DMSO-d₆) δ 8.12-8.08 (m, 1H), 7.92-7.87 (m, 1H), 7.47-7.38 (m, 1H), 7.34-7.21 (m, 3H), 6.71 (q, J=4.5 Hz, 1H), 3.97-3.82 (m, 2H), 3.82-3.68 (m, 2H), 3.68-3.51 (m, 4H), 3.31 (s, 4H), 3.22-3.04 (m, 2H), 2.48-2.35 (m, 2H); MS (ES+) m/z 500.2 (M+1).

### Example 103

### Synthesis of N-[2-(3,3-difluoropyrrolidin-1-yl)-4-(2-fluorophenyl)-3-pyridyl]-4-(1-hydroxy-1-methyl-ethyl)piperidine-1-carboxamide

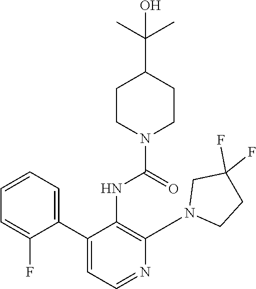 [SMILES: CC(C)(O)C1CCN(C(=O)Nc2c(-c3ccccc3F)ccnc2N2CCC(F)(F)C2)CC1]

To 2-(3,3-difluoropyrrolidin-1-yl)-4-(2-fluorophenyl)pyridin-3-amine (0.28 g, 0.95 mmol) was added anhydrous tetrahydrofuran (9.5 mL) and cooled to 0° C. To the mixture was added triphosgene (0.22 g, 0.75 mmol) and the solution was stirred at 0° C. for 2 h before 2-(piperidin-4-yl)propan-2-ol hydrochloride (0.34 g, 1.9 mmol), anhydrous tetrahydrofuran (2.0 mL) and N-ethyl-N-isopropylpropan-2-amine (1.2 g, 9.5 mmol) were added. The reaction mixture was allowed to warm to ambient temperature and stir for 72 h. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with 15-100% ethyl acetate in heptane, provided the title compound as a colorless solid (0.44 g, 99% yield): ¹H-NMR (300 MHz; DMSO-d₆) δ 8.07 (d, J=5.0 Hz, 1H), 7.81 (s, 1H), 7.40-7.16 (m, 4H), 6.68 (dd, J=5.0, 1.0 Hz, 1H), 4.10-4.07 (m, 1H), 3.93-3.86 (m, 4H), 3.78-3.73 (m, 2H), 2.48-2.36 (m, 3H), 1.53-1.49 (m, 2H), 1.30-1.11 (m, 2H), 0.98-0.95 (m, 6H), 0.74-0.66 (m, 2H); MS (ES+) m/z 463.3 (M+1).

### Example 104

### Synthesis of N-[2-(3,3-difluoropyrrolidin-1-yl)-4-(2-fluorophenyl)-3-pyridyl]-4-(1-methoxy-1-methyl-ethyl)piperidine-1-carboxamide

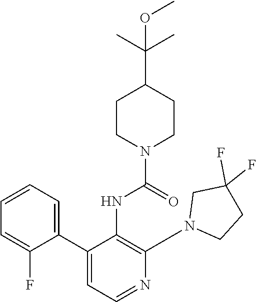 [SMILES: COC(C)(C)C1CCN(C(=O)Nc2c(-c3ccccc3F)ccnc2N2CCC(F)(F)C2)CC1]

To a mixture of iodomethane (0.41 g, 2.9 mmol), anhydrous tetrahydrofuran (2.7 mL), and 60% dispersion of sodium hydride in mineral oil (0.038 g, 0.94 mmol) was added N-[2-(3,3-difluoropyrrolidin-1-yl)-4-(2-fluorophenyl)-3-pyridyl]-4-(1-hydroxy-1-methyl-ethyl)piperidine-1-carboxamide (0.45 g, 0.97 mmol) in anhydrous tetrahydrofuran (3.0 mL) at ambient temperature. The reaction mixture was stirred at 50° C. for 30 min. After cooling to ambient temperature, the reaction mixture was diluted with ethyl acetate (150 mL), washed with saturated ammonium chloride (2×50 mL), dried over anhydrous magnesium sulfate, filtered, and concentrated in vacuo.

Purification by column chromatography, eluting with 8-100% ethyl acetate in heptane, provided the title compound as a colorless solid (0.44 g, 99% yield): ¹H-NMR (300 MHz; DMSO-d₆) δ 8.13 (d, J=4.9 Hz, 1H), 7.47-7.40 (m, 1H), 7.31-7.16 (m, 3H), 6.72 (d, J=4.7 Hz, 1H), 4.06 (d, J=3.8 Hz, 1H), 3.89-3.59 (m, 4H), 3.34-3.21 (m, 2H), 2.91 (s, 3H), 2.53-2.39 (m, 2H), 1.47-1.42 (m, 2H), 1.14-1.08 (m, 1H), 1.02-0.73 (m, 9H); MS (ES+) m/z 477.2 (M+1).

### Example 105

### Synthesis of N-[2-(3,3-difluoropyrrolidin-1-yl)-4-(2-fluorophenyl)-3-pyridyl]-4-(2,2-dimethylpropyl)-3-oxo-piperazine-1-carboxamide

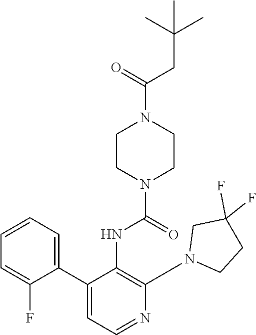 [SMILES: CC(C)(C)CC(=O)N1CCN(C(=O)Nc2c(-c3ccccc3F)ccnc2N2CCC(F)(F)C2)CC1]

To 2-(3,3-difluoropyrrolidin-1-yl)-4-(2-fluorophenyl)pyridin-3-amine (0.10 g, 0.34 mmol) was added anhydrous tetrahydrofuran (3.4 mL) and cooled to 0° C. To the mixture was added triphosgene (0.080 g, 0.27 mmol) and the solution was stirred at 0° C. for 3 h before 1-(2,2-dimethylpropyl)piperizin-2-one (0.12 g, 0.68 mmol), anhydrous tetrahydrofuran (2.0 mL) and N-ethyl-N-isopropylpropan-2-amine (0.44 g, 3.4 mmol) were added. The reaction was allowed to warm to ambient temperature and stir for 2 h.

The reaction mixture was diluted with ethyl acetate (150 mL), washed with saturated ammonium chloride (2×50 mL), dried over anhydrous magnesium sulfate, filtered, and concentrated in vacuo. Purification by column chromatography, eluting with 40-100% ethyl acetate in heptane, provided the title compound as a colorless solid (0.086 g, 51% yield): ¹H-NMR (300 MHz; DMSO-d₆) δ 8.10 (d, J=4.9 Hz, 1H), 8.03 (s, 1H), 7.42-7.35 (m, 1H), 7.29-7.14 (m, 3H), 6.70 (dd, J=5.0, 0.9 Hz, 1H), 3.96-3.85 (m, 2H), 3.85-3.72 (m, 4H), 3.34-3.32 (m, 4H), 3.14 (dd, J=9.7, 4.7 Hz, 2H), 3.10 (d, J=7.4 Hz, 2H), 2.49-2.36 (m, 2H), 0.86 (s, 9H); MS (ES+) m/z 490.2 (M+1).

### Example 106

### Synthesis of 8-bromo-N-[2-(3,3-difluoropyrrolidin-1-yl)-4-(2-fluorophenyl)-3-pyridyl]-3,4-dihydro-1H-isoquinoline-2-carboxamide

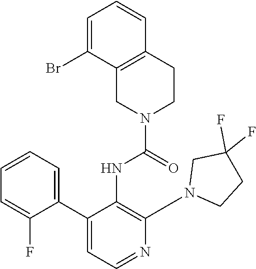 [SMILES: O=C(Nc1c(-c2ccccc2F)ccnc1N1CCC(F)(F)C1)N1CCc2cccc(Br)c2C1]

To 2-(3,3-difluoropyrrolidin-1-yl)-4-(2-fluorophenyl)pyridin-3-amine (0.30 g, 1.0 mmol) was added anhydrous tetrahydrofuran (10 mL) and the solution was cooled to 0° C. To the mixture was added triphosgene (0.19 g, 0.64 mmol) and the solution was stirred at 0° C. for 3 h. The resulting solution (1.25 mL) was added 8-bromo-1,2,3,4-tetrahydroisoquinoline hydrochloride (0.047 g, 0.19 mmol), anhydrous tetrahydrofuran (1.0 mL) and N-ethyl-N-isopropylpropan-2-amine (0.14 g, 1.1 mmol). The reaction mixture was allowed to warm to ambient temperature and stir for 2 h. The reaction mixture was diluted with ethyl acetate (20 mL), washed with saturated ammonium chloride (10 mL), dried over anhydrous magnesium sulfate, filtered, and concentrated in vacuo. Purification by preparative HPLC, eluting with 10 to 95% of acetonitrile in water containing 0.5% formic acid, provided the title compound as a colorless solid (0.029 g, 42% yield): ¹H-NMR (400 MHz; DMSO-d₆) δ 8.25 (s, 1H), 8.09 (d, J=4.9 Hz, 1H), 7.45 (dd, J=7.5, 1.6 Hz, 1H), 7.19-7.08 (m, 4H), 6.99 (t, J=9.2 Hz, 1H), 6.92 (td, J=7.5, 1.1 Hz, 1H), 6.68 (dd, J=5.0, 0.7 Hz, 1H), 4.30 (s, 2H), 4.06-3.86 (m, 2H), 3.86-3.72 (m, 2H), 3.47-3.38 (m, 2H), 2.50-2.36 (m, 4H); MS (ES+) m/z 531.2 (M+1), 533.0 (M+1).

### Example 107-132

In a similar manner as described in Example 106, utilizing the appropriately substituted starting materials and intermediates, the following compounds were prepared:

| | | | Yield % | |
|---|---|---|---|---|
| Example | | | MS (ES+) | |
| No. | Structure | Name | m/z | ¹H-NMR |
| 107 | 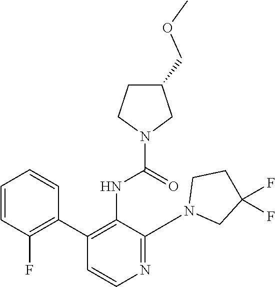 [SMILES: COC[C@H]1CCN(C(=O)Nc2c(-c3ccccc3F)ccnc2N2CCC(F)(F)C2)C1] | (3S)-N-[2- (3,3- difluoropyrrolidin- 1-yl)-4-(2- fluorophenyl)- 3-pyridyl]-3- (methoxymethyl) pyrrolidine-1- carboxamide | 32% 435.2 (M + 1) | (400 MHz; DMSO-d₆) δ 8.08 (d, J = 4.9 Hz, 1H), 7.54 (s, 1H), 7.42- 7.39 (m, 1H), 7.32-7.18 (m, 3H), 6.69 (dd, J = 5.0, 1.1 Hz, 1H), 3.98- 3.91 (m, 2H), 3.78 (t, J = 7.3 Hz, 2H), 3.22 (s, 3H), 3.19-3.03 (m, 5H), 2.82 (s, 1H), 2.44 (dt, J = 14.2, 7.1 Hz, 2H), 2.25 (t, J = 6.8 Hz, 1H), 1.79 (d, J = 6.9 Hz, 1H), 1.46 (t, J = 6.2 Hz, 1H) |
| 108 | 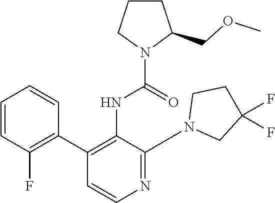 [SMILES: COC[C@@H]1CCCN1C(=O)Nc1c(-c2ccccc2F)ccnc1N1CCC(F)(F)C1] | (2S)-N-[2-(3,3- difluoropyrrolidin- 1-yl)-4-(2- fluorophenyl)- 3-pyridyl]-2- (methoxymethyl) pyrrolidine-1- carboxamide | 41% 435.2 (M + 1) | (400 MHz; DMSO-d₆) δ 8.09 (t, J = 4.2 Hz, 1H), 7.59-7.55 (m, 1H), 7.46-7.38 (m, 2H), 7.35-7.19 (m, 4H), 6.72-6.70 (m, 1H), 4.02-3.88 (m, 2H), 3.85-3.66 (m, 3H), 3.18 (s, 3H), 3.14-2.95 (m, 4H), 2.50-2.37 (m, 2H), 1.72-1.55 (m, 4H) |
| 109 | 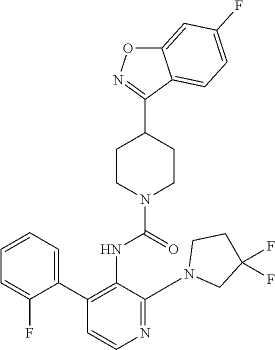 [SMILES: O=C(Nc1c(-c2ccccc2F)ccnc1N1CCC(F)(F)C1)N1CCC(c2noc3cc(F)ccc23)CC1] | N-[2-(3,3- difluoropyrrolidin- 1-yl)-4-(2- fluorophenyl)- 3-pyridyl]-4- (6-fluoro-1,2- benzoxazol-3- yl)piperidine- 1-carboxamide | 32% 540.2 (M + 1) | (400 MHz; DMSO-d₆) δ 8.10 (d, J = 4.9 Hz, 1H), 7.97 (s, 1H), 7.92 (dd, J = 8.8, 5.3 Hz, 1H), 7.71 (dd, J = 9.1, 1.9 Hz, 1H), 7.35-7.27 (m, 3H), 7.24-7.16 (m, 2H), 6.70 (dd, J = 5.0, 0.9 Hz, 1H), 3.98-3.92 (m, 3H), 3.83-3.74 (m, 2H), 3.36-3.28 (m, 2H), 2.86-2.80 (m, 2H), 2.49-2.40 (m, 2H), 1.89-1.85 (m, 2H), 1.37-1.27 (m, 2H) |
| 110 | 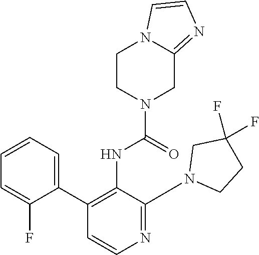 [SMILES: O=C(Nc1c(-c2ccccc2F)ccnc1N1CCC(F)(F)C1)N1CCn2ccnc2C1] | N-[2-(3,3- difluoropyrrolidin- 1-yl)-4-(2- fluorophenyl)- 3-pyridyl]-6,8- dihydro-5H- imidazo[1,2-a] pyrazine-7- carboxamide | 28% 443.4 (M + 1) | (400 MHz; DMSO-d₆): δ 8.30 (s, 1H), 8.11 (d, J = 4.9 Hz, 1H), 7.25-7.15 (m, 2H), 7.05 (d, J = 1.3 Hz, 1H), 7.04- 6.99 (m, 1H), 6.94-6.90 (m, 2H), 6.71 (dd, J = 4.9, 0.7 Hz, 1H), 4.47- 4.44 (m, 2H), 3.98-3.84 (m, 2H), 3.82-3.68 (m, 2H), 3.64-3.51 (m, 4H), 2.50-2.38 (m, 2H) |
| 111 | 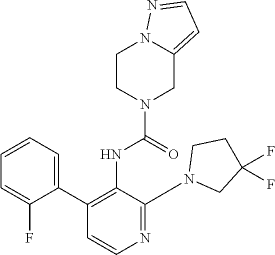 [SMILES: O=C(Nc1c(-c2ccccc2F)ccnc1N1CCC(F)(F)C1)N1CCn2nccc2C1] | N-[2-(3,3- difluoropyrrolidin- 1-yl)-4-(2- fluorophenyl)- 3-pyridyl]-6,7- dihydro-4H- pyrazolo[1,5-a] pyrazine-5- carboxamide | 22% 443.2 (M + 1) | (400 MHz; DMSO-d₆) δ 8.30 (s, 1H), 8.11 (d, J = 5.0 Hz, 1H), 7.42-7.39 (m, 1H), 7.25-7.15 (m, 3H), 7.08-7.03 (m, 1H), 6.93 (td, J = 7.5, 1.1 Hz, 1H), 6.71 (dd, J = 5.0, 0.8 Hz, 1H), 6.04-6.03 (m, 1H), 4.53-4.47 (m, 2H), 3.99-3.85 (m, 2H), 3.81-3.72 (m, 2H), 3.72-3.58 (m, 4H), 2.48-2.37 (m, 2H) |
| 112 | 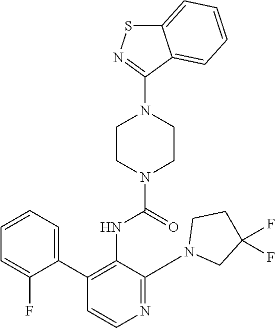 [SMILES: O=C(Nc1c(-c2ccccc2F)ccnc1N1CCC(F)(F)C1)N1CCN(c2nsc3ccccc23)CC1] | 4-(1,2- benzothiazol- 3-yl)-N-[2- (3,3- difluoropyrrolidin- 1-yl)-4-(2- fluorophenyl)-3- pyridyl]piperazine-1-carboxamide | 39% 539.2 (M + 1) | (400 MHz; DMSO-d₆) δ 8.11 (d, J = 4.9 Hz, 1H), 8.08-8.04 (m, 3H), 7.57 (ddd, J= 8.2, 7.1, 1.0 Hz, 1H), 7.44 (ddd, J = 8.2, 7.1, 1.0 Hz, 1H), 7.37-7.19 (m, 4H), 6.72 (dd, J = 4.9, 0.8 Hz, 1H), 4.01-3.80 (m, 2H), 3.79-3.78 (m, 2H), 3.43 (d, J = 0.3 Hz, 4H), 3.17 (t, J = 4.8 Hz, 4H), 2.45 (dd, J = 14.2, 7.0 Hz, 2H) |
| 113 | 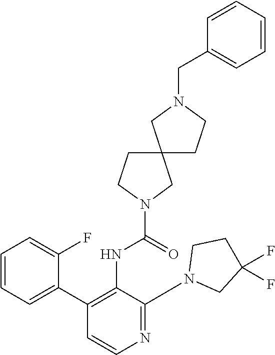 [SMILES: O=C(Nc1c(-c2ccccc2F)ccnc1N1CCC(F)(F)C1)N1CCC2(CCN(Cc3ccccc3)C2)C1] | 7-benzyl-N-[2- (3,3- difluoropyrrolidin- 1-yl)-4-(2- fluorophenyl)- 3-pyridyl]-2,7- diazaspiro[4.4] nonane-2- carboxamide | 18% 536.2 (M + 1) | (400 MHz; DMSO-d₆) δ 8.08 (t, J = 4.4 Hz, 1H), 7.57-7.52 (m, 1H), 7.35-7.23 (m, 7H), 7.20-7.11 (m, 1H), 7.11-6.99 (m, 1H), 6.68-6.65 (m, 1H), 4.02-3.86 (m, 2H), 3.81-3.72 (m, 2H), 3.61-3.49 (m, 2H), 3.17-3.03 (m, 2H), 3.02-2.89 (m, 2H), 2.63-2.53 (m, 1H), 2.48-2.33 (m, 3H), 2.29-2.12 (m, 2H), 1.80-1.64 (m, 2H), 1.63-1.44 (m, 2H) |
| 114 | 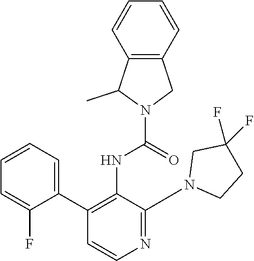 [SMILES: CC1c2ccccc2CN1C(=O)Nc1c(-c2ccccc2F)ccnc1N1CCC(F)(F)C1] | N-[2-(3,3- difluoropyrrolidin- 1-yl)-4-(2- fluorophenyl)- 3-pyridyl]- 1- methyl- isoindoline-2- carboxamide | 25% 453.2 (M + 1) | (400 MHz; DMSO-d₆) δ 8.12 (d, J = 5.0 Hz, 1H), 7.77 (s, 1H), 7.38- 7.29 (m, 2H), 7.28-7.22 (m, 5H), 7.14 (td, J = 7.4, 1.2 Hz, 1H), 6.72 (dd, J = 5.0, 1.1 Hz, 1H), 4.89-4.87 (m, 1H), 4.50-4.40 (m, 2H), 4.03-3.92 (m, 2H), 3.84-3.78 (m, 2H), 2.48-2.36 (m, 2H), 1.19-1.06 (m, 3H) |
| 115 | 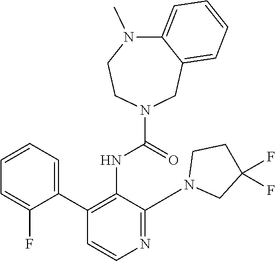 [SMILES: CN1CCN(C(=O)Nc2c(-c3ccccc3F)ccnc2N2CCC(F)(F)C2)Cc2ccccc21] | N-[2-(3,3- difluoropyrrolidin- 1-yl)-4-(2- fluorophenyl)- 3-pyridyl]-1- methyl-3,5- dihydro-2H-1,4- | 21% 482.2 (M + 1) | (400 MHz; DMSO-d₆) δ 8.06 (t, J = 4.3 Hz, 1H), 7.77-7.75 (m, 1H), 7.29-7.14 (m, 3H), 7.08-6.98 (m, 2H), 6.91-6.81 (m, 2H), 6.70-6.63 (m, 2H), 4.30-4.23 (m, 2H), 3.90-3.73 (m, 2H), 3.73-3.58 (m, 2H), 3.47-3.42 (m, 2H), 2.80-2.74 (m, 3H), 2.66-2.57 (m, 2H), 2.40-2.25 (m, 2H) |
| 116 | 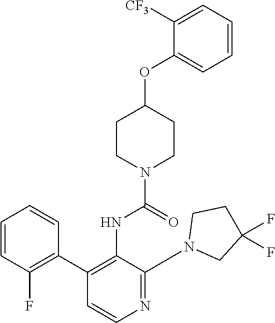 [SMILES: O=C(Nc1c(-c2ccccc2F)ccnc1N1CCC(F)(F)C1)N1CCC(Oc2ccccc2C(F)(F)F)CC1] | N-[2-(3,3- difluoropyrrolidin- 1-yl)-4-(2- fluorophenyl)- 3-pyridyl]-4- [2- (trifluoromethyl) phenoxy]piperidin- 1-carboxamide | 14% 565.2 (M + 1) | (400 MHz; DMSO-d₆) δ 8.12-8.09 (m, 1H), 8.04-7.99 (m, 1H), 7.66-7.58 (m, 2H), 7.42-7.34 (m, 1H), 7.34-7.18 (m, 4H), 7.09-7.04 (m, 1H), 6.71 (td, J = 4.6, 0.8 Hz, 1H), 4.73-4.64 (m, 1H), 4.05-3.84 (m, 2H), 3.83-3.69 (m, 2H), 3.58-3.42 (m, 2H), 3.21-3.05 (m, 2H), 2.48-2.36 (m, 2H), 1.66-1.54 (m, 2H), 1.30-1.19 (m, 2H) |
| 117 | 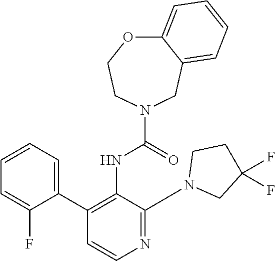 [SMILES: O=C(Nc1c(-c2ccccc2F)ccnc1N1CCC(F)(F)C1)N1CCOc2ccccc2C1] | N-[2-(3,3- difluoropyrrolidin- 1-yl)-4-(2- fluorophenyl)- 3-pyridyl]-3,5- dihydro-2H- 1,4- benzoxazepine-4- carboxamide | 23% 469.2 (M + 1) | (400 MHz; DMSO-d₆) δ 8.06 (dd, J = 4.7, 2.9 Hz, 1H), 7.95-7.91 (m, 1H), 7.40-7.32 (m, 1H), 7.27-7.20 (m, 1H), 7.18-7.09 (m, 1H), 7.06-6.88 (m, 4H), 6.67-6.58 (m, 2H), 4.43-4.35 (m, 2H), 4.00-3.51 (m, 6H), 3.40-3.33 (m, 2H), 2.41-2.26 (m, 2H) |
| 118 | 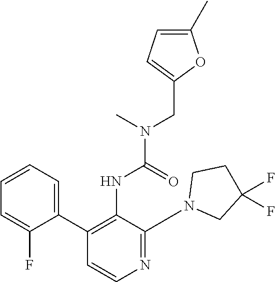 [SMILES: Cc1ccc(CN(C)C(=O)Nc2c(-c3ccccc3F)ccnc2N2CCC(F)(F)C2)o1] | 3-[2-(3,3- difluoropyrrolidin- 1-yl)-4-(2- fluorophenyl)- 3-pyridyl]-1- methyl-1-[(5- methyl-2- furyl)methyl]urea | 23% 445.2 (M + 1) | (400 MHz; DMSO-d₆) δ 8.09 (d, J = 5.0 Hz, 1H), 7.83 (s, 1H), 7.40 (dddd, J = 8.3, 7.2, 5.4, 1.9 Hz, 1H), 7.33-7.21 (m, 2H), 7.15 (td, J = 7.5, 1.1 Hz, 1H), 6.70- 6.68 (m, 1H), 5.94-5.93 (m, 1H), 5.79 (d, J = 3.0 Hz, 1H), 4.22 (s, 2H), 3.98-3.85 (m, 2H), 3.78-3.72 (m, 2H), 2.65 (s, 3H), 2.49-2.35 (m, 2H), 2.19 (s, 3H) |
| 119 | 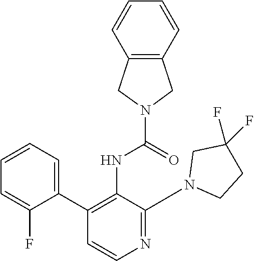 [SMILES: O=C(Nc1c(-c2ccccc2F)ccnc1N1CCC(F)(F)C1)N1Cc2ccccc2C1] | N-[2-(3,3- difluoropyrrolidin- 1-yl)-4-(2- fluorophenyl)-3- pyridyl]isoindoline- 2-carboxamide | 61% 439.2 (M + 1) | (400 MHz; DMSO-d₆) δ 8.12 (d, J = 5.0 Hz, 1H), 7.85 (s, 1H), 7.38- 7.30 (m, 3H), 7.30-7.26 (m, 3H), 7.27-7.20 (m, 1H), 7.15 (td, J = 7.5, 1.2 Hz, 1H), 6.74-6.73 (m, 1H), 4.47-4.39 (m, 4H), 4.03-3.94 (m, 2H), 3.85-3.79 (m, 2H), 2.48-2.39 (m, 2H) |
| 120 | 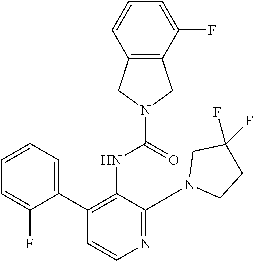 [SMILES: O=C(Nc1c(-c2ccccc2F)ccnc1N1CCC(F)(F)C1)N1Cc2cccc(F)c2C1] | N-[2-(3,3- difluoropyrrolidin- 1-yl)-4-(2- fluorophenyl)- 3-pyridyl]-4- fluoro- isoindoline-2- carboxamide | 40% 457.2 (M + 1) | (400 MHz; DMSO-d₆) δ 8.12 (d, J = 5.0 Hz, 1H), 7.93 (d, J = 4.4 Hz, 1H), 7.38-7.30 (m, 3H), 7.27-7.21 (m, 1H), 7.19-7.08 (m, 3H), 6.75-6.73 (m, 1H), 4.53-4.42 (m, 4H), 4.03-3.93 (m, 2H), 3.86-3.78 (m, 2H), 2.49-2.38 (m, 2H |
| 121 | 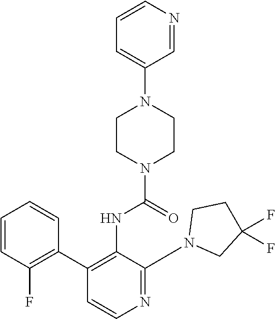 [SMILES: O=C(Nc1c(-c2ccccc2F)ccnc1N1CCC(F)(F)C1)N1CCN(c2cccnc2)CC1] | N-[2-(3,3- difluoropyrrolidin- 1-yl)-4-(2- fluorophenyl)- 3-pyridyl]-4- (3-pyridyl) piperazine-1- carboxamide | 39% 483.2 (M + 1) | (400 MHz; DMSO-d₆) δ 8.30-8.21 (m, 1H), 8.12-8.06 (m, 2H), 8.06-8.00 (m, 1H), 7.33-7.19 (m, 5H), 7.19-7.11 (m, 1H), 6.71-6.68 (m, 1H), 4.07-3.85 (m, 2H), 3.85-3.71 (m, 2H), 3.35 (s, 4H), 2.89 (s, 4H), 2.49-2.38 (m, 2H) |
| 122 | 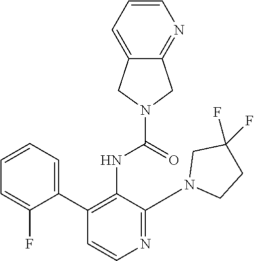 [SMILES: O=C(Nc1c(-c2ccccc2F)ccnc1N1CCC(F)(F)C1)N1Cc2cccnc2C1] | N-[2-(3,3- difluoropyrrolidin- 1-yl)-4-(2- fluorophenyl)- 3-pyridyl]-5,7- dihydropyrrolo [3,4-b]pyridine-6-carboxamide | 50% 440.2 (M + 1) | (400 MHz; DMSO-d₆) δ 8.47-8.40 (m, 1H), 8.16-8.09 (m, 1H), 7.99-7.88 (m, 1H), 7.76-7.67 (m, 1H), 7.40-7.26 (m, 3H), 7.25-7.19 (m, 1H), 7.19-7.09 (m, 1H), 6.78-6.64 (m, 1H), 4.54-4.27 (m, 4H), 4.08-3.91 (m, 2H), 3.89-3.78 (m, 2H), 2.51-2.36 (m, 2H) |
| 123 | 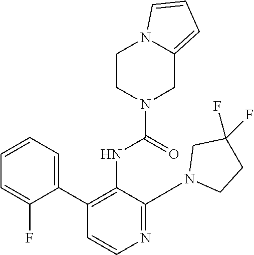 [SMILES: O=C(Nc1c(-c2ccccc2F)ccnc1N1CCC(F)(F)C1)N1CCn2cccc2C1] | N-[2-(3,3- difluoropyrrolidin- 1-yl)-4-(2- fluorophenyl)-3-pyridyl]-3,4- dihydro-1H- pyrrolo[1,2-a] pyrazine-2- carboxamide | 50% 442.2 (M + 1) | (400 MHz; DMSO-d₆) δ 8.20-8.13 (m, 1H), 8.13-8.06 (m, 1H), 7.32-7.24 (m, 1H), 7.24-7.16 (m, 1H), 7.12-7.04 (m, 1H), 7.00-6.91 (m, 1H), 6.74-6.69 (m, 1H), 6.61-6.56 (m, 1H), 6.02-5.96 (m, 1H), 5.78-5.72 (m, 1H), 4.44-4.34 (m, 2H), 4.02-3.83 (m, 2H), 3.82-3.66 (m, 2H), 3.62-3.45 (m, 4H), 2.48-2.35 (m, 2H) |
| 124 | 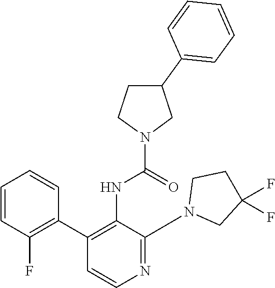 [SMILES: O=C(Nc1c(-c2ccccc2F)ccnc1N1CCC(F)(F)C1)N1CCC(c2ccccc2)C1] | N-[2-(3,3- difluoropyrrolidin- 1-yl)-4-(2- fluorophenyl)- 3-pyridyl]-3- phenyl- pyrrolidine-1- carboxamide | 54% 467.2 (M + 1) | (400 MHz; DMSO-d₆) δ 8.14-8.06 (m, 1H), 7.66-7.59 (m, 1H), 7.50-7.42 (m, 1H), 7.37-7.30 (m, 3H), 7.30-7.20 (m, 3H), 7.19-7.12 (m, 2H), 6.74-6.68 (m, 1H), 4.07-3.91 (m, 2H), 3.86-3.74 (m, 2H), 3.59-3.46 (m, 1H), 3.30-3.24 (m, 1H), 3.22-3.08 (m, 2H), 3.06-2.96 (m, 1H), 2.51-2.37 (m, 2H), 2.19-2.06 (m, 1H), 1.81-1.68 (m, 1H) |
| 125 | 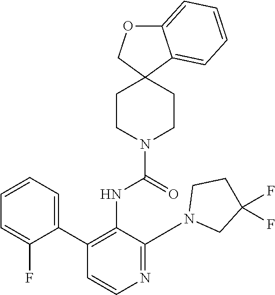 [SMILES: O=C(Nc1c(-c2ccccc2F)ccnc1N1CCC(F)(F)C1)N1CCC2(CC1)COc1ccccc12] | N-[2-(3,3- difluoropyrrolidin- 1-yl)-4-(2- fluorophenyl)-3- pyridyl]spiro[2H- benzofuran- 3,4′- piperidine]-1′-carboxamide | 46% 509.2 (M + 1) | (400 MHz; DMSO-d₆) δ 8.13-8.08 (m, 1H), 8.06-8.00 (m, 1H), 7.51-7.42 (m, 1H), 7.34-7.23 (m, 3H), 7.21-7.15 (m, 1H), 7.11-7.04 (m, 1H), 6.85-6.77 (m, 1H), 6.77-6.67 (m, 2H), 4.11-3.85 (m, 2H), 3.85-3.67 (m, 2H), 3.57-3.37 (m, 2H), 3.31-3.13 (m, 2H), 2.94-2.83 (m, 2H), 2.51-2.38 (m, 2H), 1.51-1.42 (m, 2H), 1.29-1.13 (m, 2H) |
| 126 | 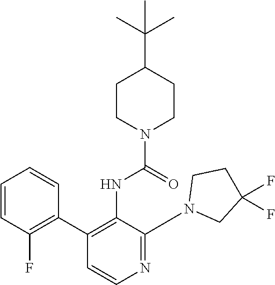 [SMILES: CC(C)(C)C1CCN(C(=O)Nc2c(-c3ccccc3F)ccnc2N2CCC(F)(F)C2)CC1] | 4-tert-butyl-N- [2-(3,3-difluoropyrrolidin- 1-yl)-4-(2- fluorophenyl)-3- pyridyl]piperidine- 1-carboxamide | 50% 461.4 (M + 1) | (400 MHz; DMSO-d₆) δ 8.08 (dd, J = 4.9, 1.8 Hz, 1H), 7.82 (s, 1H), 7.40-7.34 (m, 1H), 7.31-7.17 (m, 3H), 6.67 (d, J = 4.8 Hz, 1H), 3.95-3.85 (m, 4H), 3.76-3.74 (m, 2H), 2.51-2.38 (m, 4H), 1.47-1.44 (m, 2H), 1.08-1.02 (m, 1H), 0.77-0.73 (m, 9H), 0.71-0.60 (m, 2H) |
| 127 | 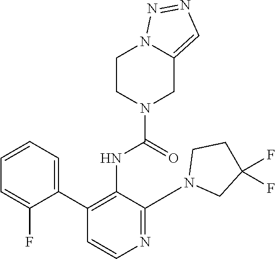 [SMILES: O=C(Nc1c(-c2ccccc2F)ccnc1N1CCC(F)(F)C1)N1CCn2nncc2C1] | N-[2-(3,3- difluoropyrrolidin- 1-yl)-4-(2- fluorophenyl)- 3-pyridyl]-6,7- dihydro-4H- triazolo[1,5-a] pyrazine-5- carboxamide | 64% 444.2 (M + 1) | (400 MHz; DMSO-d₆) δ 8.41-8.39 (m, 1H), 8.13-8.09 (m, 1H), 7.58-7.56 (m, 1H), 7.19-7.10 (m, 2H), 7.03-6.95 (m, 1H), 6.87-6.82 (m, 1H), 6.70 (t, J = 4.7 Hz, 1H), 4.64-4.54 (m, 2H), 4.08-3.81 (m, 4H), 3.81-3.54 (m, 4H), 2.51-2.37 (m, 2H) |
| 128 | 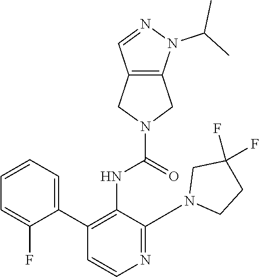 [SMILES: CC(C)n1ncc2c1CN(C(=O)Nc1c(-c3ccccc3F)ccnc1N1CCC(F)(F)C1)C2] | N-[2-(3,3- difluoropyrrolidin- 1-yl)-4-(2- fluorophenyl)- 3-pyridyl]-1- isopropyl-4,6- dihydropyrrolo [3,4-c] pyrazole-5-carboxamide | 9% 471.2 (M + 1) | (400 MHz; DMSO-d₆) δ 8.12 (d, J = 5.1 Hz, 1H), 7.87 (s, 1H), 7.41- 7.34 (m, 2H), 7.28-7.23 (m, 1H), 7.22 (s, 1H), 7.22-7.17 (m, 1H), 6.78-6.77 (m, 1H), 4.41-4.32 (m, 3H), 4.12-4.09 (m, 2H), 4.04-3.96 (m, 2H), 3.86-3.83 (m, 2H), 2.48-2.41 (m, 2H), 1.34 (t, J = 6.6 Hz, 6H) |
| 129 | 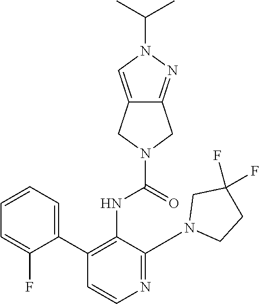 [SMILES: CC(C)n1cc2c(n1)CN(C(=O)Nc1c(-c3ccccc3F)ccnc1N1CCC(F)(F)C1)C2] | N-[2-(3,3- difluoropyrrolidin- 1-yl)-4-(2- fluorophenyl)- 3-pyridyl]-2- isopropyl-4,6- dihydropyrrolo [3,4-c] pyrazole-5-carboxamide | 7% 471.2 (M + 1) | (400 MHz; DMSO-d₆) δ 8.12 (d, J = 5.1 Hz, 1H), 7.83 (d, J = 5.9 Hz, 1H), 7.55 (s, 1H), 7.39-7.34 (m, 2H), 7.27-7.22 (m, 1H), 7.20-7.16 (m, 1H), 6.76-6.75 (m, 1H), 4.45 (dt, J = 13.3, 6.7 Hz, 1H), 4.20-4.11 (m, 4H), 4.04-3.95 (m, 2H), 3.85-3.80 (m, 2H), 2.50-2.40 (m, 2H), 1.40-1.34 (m, 6H) |
| 130 | 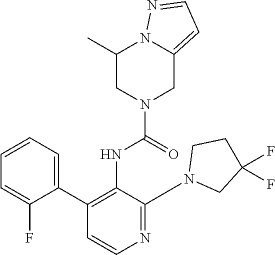 [SMILES: CC1CN(C(=O)Nc2c(-c3ccccc3F)ccnc2N2CCC(F)(F)C2)Cc2ccnn21] | N-[2-(3,3- difluoropyrrolidin- 1-yl)-4-(2- fluorophenyl)- 3-pyridyl]-7- methyl-6,7- dihydro-4H- pyrazolo[1,5-a] pyrazine-5- carboxamide | 19% 457.2 (M + 1) | (400 MHz; DMSO-d₆) δ 8.28-8.23 (m, 1H), 8.13-8.09 (m, 1H), 7.43-7.38 (m, 1H), 7.29-7.18 (m, 2H), 7.15-7.06 (m, 1H), 7.03-6.98 (m, 1H), 6.72 (t, J = 4.8 Hz, 1H), 6.01 (t, J = 2.9 Hz, 1H), 4.60-4.53 (m, 1H), 4.43-4.37 (m, 1H), 4.02-3.81 (m, 3H), 3.81-3.64 (m, 3H), 3.42-3.36 (m, 1H), 2.50-2.36 (m, 2H), 1.23-1.14 (m, 3H) |
| 131 | 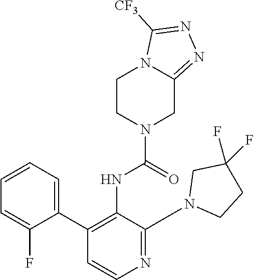 [SMILES: O=C(Nc1c(-c2ccccc2F)ccnc1N1CCC(F)(F)C1)N1CCn2c(nnc2C(F)(F)F)C1] | N-[2-(3,3- difluoropyrrolidin- 1-yl)-4-(2- fluorophenyl)- 3-pyridyl]-3- (trifluoromethyl)- 6,8- dihydro-5H- [1,2,4]triazolo [4,3-a]pyrazine-7- carboxamide | 55% 512.2 (M + 1) | (400 MHz; DMSO-d₆) δ 8.60-8.52 (m, 1H), 8.14-8.09 (m, 1H), 7.19-7.12 (m, 1H), 7.12-7.05 (m, 1H), 6.99-6.89 (m, 1H), 6.88-6.78 (m, 1H), 6.72-6.68 (m, 1H), 4.83-4.71 (m, 2H), 4.18-3.39 (m, 8H), 2.50-2.38 (m, 2H) |
| 132 | 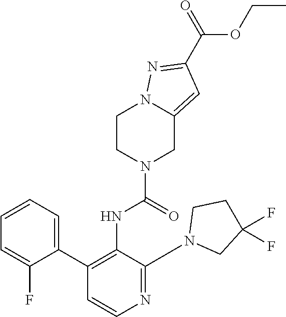 [SMILES: CCOC(=O)c1cc2n(n1)CCN(C(=O)Nc1c(-c3ccccc3F)ccnc1N1CCC(F)(F)C1)C2] | ethyl 5-[[2-(3,3- difluoropyrrolidin- 1-yl)-4-(2- fluorophenyl)- 3-pyridyl] carbamoyl]-6,7- dihydro-4H- pyrazolo[1,5-a] pyrazine-2- carboxylate | 47% 515.2 (M + 1) | (400 MHz; DMSO-d₆) δ 8.36 (d, J = 6.2 Hz, 1H), 8.10 (t, J = 4.3 Hz, 1H), 7.21-7.15 (m, 2H), 7.06-7.02 (m, 1H), 6.91 (dd, J = 9.6, 5.2 Hz, 1H), 6.71 (d, J = 4.9 Hz, 1H), 6.53 (s, 1H), 4.54-4.48 (m, 2H), 4.29 (q, J = 5.3 Hz, 2H), 3.99-3.82 (m, 2H), 3.82-3.59 (m, 6H), 2.49-2.38 (m, 2H), 1.29 (t, J = 5.3 Hz, 3H) |

### Example 133

### Synthesis of N-[2-(3,3-difluoropyrrolidin-1-yl)-4-(2-fluorophenyl)-3-pyridyl]-2-methoxy-5, 7-dihydropyrrolo[3,4-b]pyridine-6-carboxamide

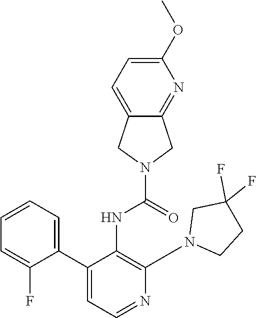 [SMILES: COc1ccc2c(n1)CN(C(=O)Nc1c(-c3ccccc3F)ccnc1N1CCC(F)(F)C1)C2]

### Step 1. Preparation of 2-chloro-N-[2-(3,3-difluoropyrrolidin-1-yl)-4-(2-fluorophenyl)-3-pyridyl]-5,7-dihydropyrrolo[3,4-b]pyridine-6-carboxamnide

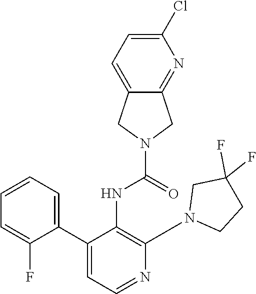 [SMILES: O=C(Nc1c(-c2ccccc2F)ccnc1N1CCC(F)(F)C1)N1Cc2ccc(Cl)nc2C1]

To 2-(3,3-difluoropyrrolidin-1-yl)-4-(2-fluorophenyl)pyridin-3-amine (0.30 g, 1.0 mmol) was added anhydrous tetrahydrofuran (10 mL) and cooled to 0° C. To the mixture was added triphosgene (0.19 g, 0.64 mmol) and the solution was stirred at 0° C. for 3 h. The resulting solution (1.25 mL) was added to 2-chloro-5H,6H,7H-pyrrolo[3,4-b]pyridine hydrochloride (0.29 g, 1.5 mmol), anhydrous tetrahydrofuran (5.0 mL) and N-ethyl-N-isopropylpropan-2-amine (0.88 g, 6.8 mmol). The reaction mixture was allowed to warm to ambient temperature and stir for 24 h. The reaction mixture was diluted with ethyl acetate (200 mL), washed with saturated ammonium chloride (2×50 mL), dried over anhydrous magnesium sulfate, filtered, and concentrated in vacuo. Purification by column chromatography, eluting with a gradient of 10 to 80% ethyl acetate in heptane, provided the title compound as a grey solid (0.26 g, 40% yield): MS (ES+) m/z 474.2 (M+1), 476.2 (M+1).

### Step 2. Preparation of N-[2-(3,3-difluoropyrrolidin-1-yl)-4-(2-fluorophenyl)-3-pyridyl]-2-methoxy-5,7-dihydropyrrolo[3,4-b]pyridine-6-carboxamide

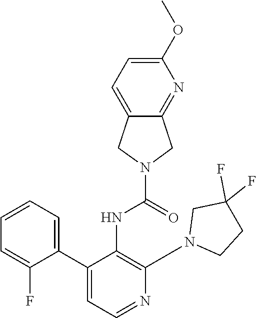 [SMILES: COc1ccc2c(n1)CN(C(=O)Nc1c(-c3ccccc3F)ccnc1N1CCC(F)(F)C1)C2]

To 2-chloro-N-[2-(3,3-difluoropyrrolidin-1-yl)-4-(2-fluorophenyl)-3-pyridyl]-5,7-dihydropyrrolo[3,4-b]pyridine-6-carboxamide (0.050 g, 0.11 mmol) was added anhydrous 1,4-dioxane (0.53 mL) and anhydrous methanol (0.034 g, 1.1 mmol). Anhydrous potassium tert-butoxide (0.12 g, 1.1 mmol) was added, the vial was sealed, and the mixture was heated to 90° C. for 3 h. The reaction mixture was cooled to ambient temperature, diluted with ethyl acetate (100 mL), washed with saturated ammonium chloride (2×50 mL), dried over anhydrous magnesium sulfate, filtered, and concentrated in vacuo. Purification by column chromatography, eluting with 0 to 100% of ethyl acetate in heptane, provided the title compound as a colorless solid (0.027 g, 50% yield): ¹H-NMR (400 MHz; DMSO-d₆) δ 58.13 (d, J=4.9 Hz, 1H), 7.87 (s, 1H), 7.63 (d, J=8.4 Hz, 1H), 7.36-7.31 (m, 2H), 7.23 (ddd, J=9.9, 8.7, 1.0 Hz, 1H), 7.16 (td, J=7.5, 1.2 Hz, 1H), 6.74-6.71 (m, 2H), 4.38-4.30 (m, 4H), 4.02-3.94 (m, 2H), 3.86-3.80 (m, 5H), 2.50-2.39 (m, 2H); MS (ES+) m/z 470.2 (M+1).

### Example 134

### Synthesis of N-(4-(2,5-difluorophenyl)-2-(3,3-difluoropyrrolidin-1-yl)pyridin-3-yl)pyrimidine-5-carboxamide

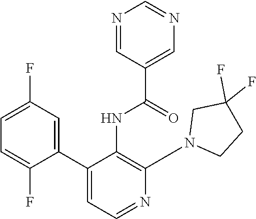 [SMILES: O=C(Nc1c(-c2cc(F)ccc2F)ccnc1N1CCC(F)(F)C1)c1cncnc1]

### Step 1. Preparation of N-(4-(2,5-difluorophenyl)-2-(3,3-difluoropyrrolidin-1-yl)pyridin-3-yl)pyrimidine-5-carboxamide

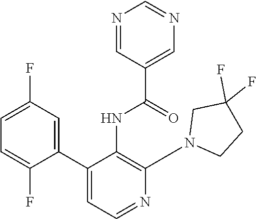 [SMILES: O=C(Nc1c(-c2cc(F)ccc2F)ccnc1N1CCC(F)(F)C1)c1cncnc1]

To a solution of 4-(2,5-difluorophenyl)-2-(3,3-difluoropyrrolidin-1-yl)pyridin-3-amine (0.050 g, 0.16 mmol), 5-pyrimidinecarboxylic acid (0.030 g, 0.24 mmol) and 2-chloro-1-methylpyridinium iodide (0.12 g, 0.48 mmol) was added anhydrous tetrahydrofuran (2.0 mL). The solution was heated at 65° C. for 5 min before N-ethyl-N-isopropylpropan-2-amine (0.21 g, 1.6 mmol) was added. The reaction mixture was stirred at 65° C. for 8 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with 25-100% ethyl acetate in heptane, followed by preparative HPLC eluting with a gradient of 10 to 90% of acetonitrile in water containing 0.5% formic acid to provide the title compound as a colorless solid (0.011 g, 17% yield): ¹H-NMR (500 MHz; DMSO-d₆) δ 10.32 (s, 1H), 9.33 (s, 1H), 8.98 (d, J=0.2 Hz, 2H), 8.24 (d, J=4.9 Hz, 1H), 7.32 (td, J=9.2, 4.5 Hz, 1H), 7.25-7.21 (m, 1H), 7.18-7.14 (m, 1H), 6.86 (d, J=5.0 Hz, 1H), 3.95-3.86 (m, 2H), 3.80-3.73 (m, 2H), 2.50-2.41 (m, 2H); MS (ES+) m/z 418.2 (M+1).

### Example 135

### Synthesis of N-(4-(2,5-difluorophenyl)-2-(3,3-difluoropyrrolidin-1-yl)pyridin-3-yl)-1-isopropyl-1H-pyrazole-4-carboxamide

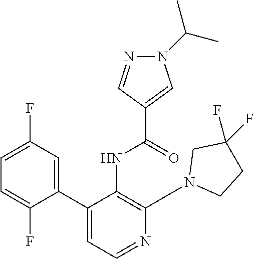 [SMILES: CC(C)n1cc(C(=O)Nc2c(-c3cc(F)ccc3F)ccnc2N2CCC(F)(F)C2)cn1]

To a solution of 4-(2,5-difluorophenyl)-2-(3,3-difluoropyrrolidin-1-yl)pyridin-3-amine (0.050 g, 0.16 mmol), 1-isopropyl-1H-pyrazole-4-carboxylic acid (0.043 g, 0.26 mmol) and 2-chloro-1-methylpyridinium iodide (0.16 g, 0.64 mmol) was added anhydrous tetrahydrofuran (2.0 mL). The solution was heated at 65° C. for 5 min before N-ethyl-N-isopropylpropan-2-amine (0.21 g, 1.6 mmol) was added. The reaction mixture was stirred at 65° C. for 18 h. The reaction mixture was cooled to ambient temperature and diluted with ethyl acetate (100 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with 25-100% ethyl acetate in heptane, followed by preparative HPLC eluting with a gradient of 10 to 90% of acetonitrile in water containing 0.5% formic acid, to provide the title compound as a colorless solid (0.012 g, 17% yield): ¹H-NMR (500 MHz; DMSO-d₆) a 9.45 (s, 1H), 8.19-8.17 (m, 2H), 7.84 (s, 1H), 7.28 (td, J=9.1, 4.6 Hz, 1H), 7.22-7.18 (m, 1H), 7.15-7.11 (m, 1H), 6.79 (d, J=4.9 Hz, 1H), 4.51-4.45 (m, 1H), 3.92-3.84 (m, 2H), 3.76-3.72 (m, 2H), 2.48-2.39 (m, 2H), 1.40 (t, J=5.8 Hz, 6H); MS (ES+) m/z 448.2 (M+1).

### Example 136

### Synthesis of N-(4-(2,5-difluorophenyl)-2-(3,3-difluoropyrrolidin-1-yl)pyridin-3-yl)-3,5-dimethylisoxazole-4-carboxamide

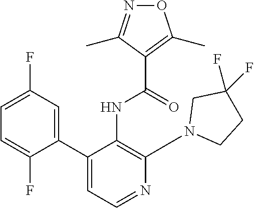 [SMILES: Cc1noc(C)c1C(=O)Nc1c(-c2cc(F)ccc2F)ccnc1N1CCC(F)(F)C1]

To a solution of 4-(2,5-difluorophenyl)-2-(3,3-difluoropyrrolidin-1-yl)pyridin-3-amine (0.10 g, 0.32 mmol), 3,5-dimethylisoxazole-4-carboxylic acid (0.068 g, 0.48 mmol) and 2-chloro-1-methylpyridinium iodide (0.33 g, 1.3 mmol) was added anhydrous tetrahydrofuran (4.0 mL). The solution was heated at 65° C. for 5 min before N-ethyl-N-isopropylpropan-2-amine (0.42 g, 3.2 mmol) was added. The reaction mixture was stirred at 65° C. for 20 h. The reaction mixture was cooled to ambient temperature and diluted with ethyl acetate (200 mL). The reaction mixture was washed with saturated ammonium chloride solution (2×50 mL). The organic layer was dried over anhydrous magnesium sulfate, filtered and concentrated in vacuo. Purification of the residue preparative HPLC eluting with a gradient of 10 to 85% of acetonitrile in water containing 0.5% formic acid, to provide the title compound as a colorless solid (0.028 g, 20% yield): ¹H-NMR (500 MHz; DMSO-d₆) δ 9.56-9.53 (m, 1H), 8.21 (d, J=4.9 Hz, 1H), 7.41-7.36 (m, 1H), 7.34-7.29 (m, 1H), 7.14-7.10 (m, 1H), 6.81 (d, J=4.9 Hz, 1H), 3.94-3.84 (m, 2H), 3.79-3.71 (m, 2H), 2.50-2.43 (m, 2H), 2.26 (s, 3H), 2.07 (s, 3H); MS (ES+) m/z 435.2 (M+1).

### Example 137

### Synthesis of N-(2-(3,3-difluoropyrrolidin-1-yl)-4-(2-fluorophenyl)pyridin-3-yl)-1-isopropyl-1H-pyrazole-4-carboxamide

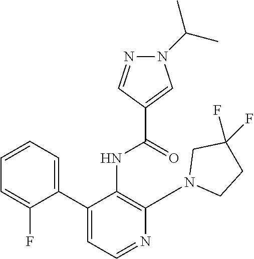 [SMILES: CC(C)n1cc(C(=O)Nc2c(-c3ccccc3F)ccnc2N2CCC(F)(F)C2)cn1]

### Step 1. Preparation of 2-chloro-4-(2-fluorophenyl)-3-nitropyridine

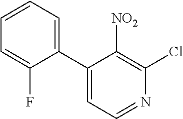 [SMILES: O=[N+]([O-])c1c(-c2ccccc2F)ccnc1Cl]

A mixture of 2,4-dichloro-3-nitropyridine (5.0 g, 26 mmol), 1,4-dioxane (50 mL), and water (17 mL) was sparged with nitrogen for 10 min. The flask was added 2-fluorophenylboronic acid (3.6 g, 26 mmol), potassium carbonate (5.4 g, 39 mmol), and [1,1′-bis(diphenylphosphino)ferrocene]dichloropalladium(II), complex with dichloromethane (2.2 g, 2.6 mmol), and sparged with nitrogen for 2 min. The reaction mixture was stirred at 60° C. for 4 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 0-30% ethyl acetate in heptane, afforded the title compound as a colorless solid (4.0 g, 61% yield).

### Step 2. Preparation of 4-(2-fluorophenyl)-2-(3,3-difluoropyrrolidin-1-yl)-3-nitropyridine

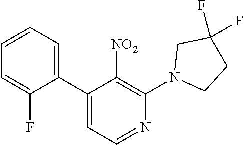 [SMILES: O=[N+]([O-])c1c(-c2ccccc2F)ccnc1N1CCC(F)(F)C1]

To a solution of 2-chloro-4-(2-fluorophenyl)-3-nitropyridine (2.0 g, 7.9 mmol), anhydrous potassium carbonate (3.3 g, 24 mmol), and 3,3-difluoropyrrolidine hydrochloride (1.5 g, 10 mmol) was charged N,N-dimethylformamide (26 mL). The reaction mixture was stirred at ambient temperature for 24 h. The reaction mixture was diluted with ethyl acetate (200 mL). The organic layer was washed with saturated ammonium chloride (2×50 mL). The organic solution was dried over anhydrous magnesium sulfate, filtered, and concentrated in vacuo. Purification of the residue by column chromatography, eluting with 5-35% ethyl acetate in heptane, afforded the title compound as a yellow oil (2.5 g, 98% yield): MS (ES+) m/z 324.2 (M+1).

### Step 3. Preparation of 2-(3,3-difluoropyrrolidin-1-yl)-4-(2-fluorophenyl)pyridin-3-amine

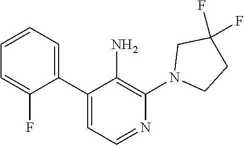 [SMILES: Nc1c(-c2ccccc2F)ccnc1N1CCC(F)(F)C1]

To a solution of 4-(2-fluorophenyl)-2-(3,3-difluoropyrrolidin-1-yl)-3-nitropyridine (2.5 g, 7.8 mmol) was added anhydrous methanol (13 mL), ethyl acetate (13 mL), and 10% palladium on carbon (0.83 g). The reaction vessel was sealed and the reaction mixture was sparged with hydrogen gas for 5 min. The reaction mixture was stirred under a hydrogen atmosphere for 24 h. The reaction mixture was filtered through diatomaceous earth (i.e., Celite®), washed with ethyl acetate (5×20 mL) and concentrated in vacuo. Purification of the residue by column chromatography, eluting with 5-35% ethyl acetate in heptane, afforded the title compound as a clear colorless oil (1.6 g, 72% yield): MS (ES+) m/z 294.2 (M+1).

### Step 4. Preparation of N-(2-(3,3-difluoropyrrolidin-1-yl)-4-(2-fluorophenyl)pyridin-3-yl)-1-isopropyl-1H-pyrazole-4-carboxamide

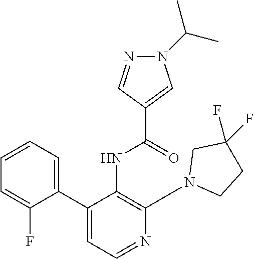 [SMILES: CC(C)n1cc(C(=O)Nc2c(-c3ccccc3F)ccnc2N2CCC(F)(F)C2)cn1]

To a solution of 2-(3,3-difluoropyrrolidin-1-yl)-4-(2-fluorophenyl)pyridin-3-amine (0.10 g, 0.34 mmol), 1-isopropyl-1H-pyrazole-4-carboxylic acid (0.089 g, 0.58 mmol) and 2-chloro-1-methylpyridinium iodide (0.35 g, 1.4 mmol) was added anhydrous tetrahydrofuran (4.3 mL). The solution was heated at 65° C. for 5 min before N-ethyl-N-isopropylpropan-2-amine (0.44 g, 3.4 mmol) was added. The reaction mixture was stirred at 65° C. for 20 h. The reaction mixture was cooled to ambient temperature and diluted with ethyl acetate (150 mL). The reaction mixture was washed with saturated ammonium chloride solution (2×50 mL). The organic layer was dried over anhydrous magnesium sulfate, filtered and concentrated in vacuo. Purification of the residue column chromatography, eluting with a gradient of 15 to 100% of ethyl acetate in heptane, to provide the title compound as a colorless solid (0.039 g, 27% yield): ¹H-NMR (500 MHz; DMSO-d₆) δ 9.39 (s, 1H), 8.16 (d, J=5.0 Hz, 1H), 8.13 (s, 1H), 7.81 (s, 1H), 7.37-7.28 (m, 2H), 7.23-7.20 (m, 1H), 7.15 (td, J=7.5, 0.9 Hz, 1H), 6.76 (d, J=4.9 Hz, 1H), 4.47 (dt, J=13.3, 6.6 Hz, 1H), 3.92-3.84 (m, 2H), 3.75-3.72 (m, 2H), 2.47-2.38 (m, 2H), 1.38 (d, J=6.7 Hz, 6H); MS (ES+) m/z 430.2 (M+1).

### Example 138

### Synthesis of N-(4-(2,5-difluorophenyl)-2-(3,3-difluoropyrrolidin-1-yl)pyridin-3-yl)-2-methoxypyrimidine-5-carboxamide

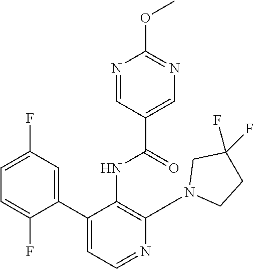 [SMILES: COc1ncc(C(=O)Nc2c(-c3cc(F)ccc3F)ccnc2N2CCC(F)(F)C2)cn1]

To a solution of 4-(2,5-difluorophenyl)-2-(3,3-difluoropyrrolidin-1-yl)pyridin-3-amine (0.075 g, 0.24 mmol), 2-methoxypyrimidine-5-carboxylic acid (0.056 g, 0.36 mmol) and 2-chloro-1-methylpyridinium iodide (0.25 g, 0.96 mmol) was added anhydrous tetrahydrofuran (3.0 mL). The solution was heated at 65° C. for 5 min before N-ethyl-N-isopropylpropan-2-amine (0.31 g, 2.4 mmol) was added. The reaction mixture was stirred at 65° C. for 2 h. The reaction mixture was cooled to ambient temperature and diluted with ethyl acetate (200 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with a gradient of 15 to 100% of ethyl acetate in heptane, followed preparative HPLC eluting with a gradient of 10 to 70% of acetonitrile in water containing 0.5% formic acid to provide the title compound as a colorless solid (0.042 g, 38% yield): ¹H-NMR (500 MHz; DMSO-d₆) δ 10.10 (s, 1H), 8.86-8.84 (m, 2H), 8.23 (d, J=4.9 Hz, 1H), 7.33-7.29 (m, 1H), 7.25-7.20 (m, 1H), 7.16-7.13 (m, 1H), 6.84 (d, J=5.0 Hz, 1H), 3.95 (s, 3H), 3.95-3.81 (m, 2H), 3.82-3.71 (m, 2H), 2.48-2.40 (m, 2H); MS (ES+) m/z 448.0 (M+1).

### Example 139

### Synthesis of N-(4-(2,5-difluorophenyl)-2-(3,3-difluoropyrrolidin-1-yl)pyridin-3-yl)pyridazine-4-carboxamide

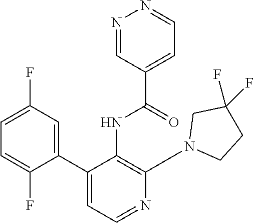 [SMILES: O=C(Nc1c(-c2cc(F)ccc2F)ccnc1N1CCC(F)(F)C1)c1ccnnc1]

To a solution of 4-(2,5-difluorophenyl)-2-(3,3-difluoropyrrolidin-1-yl)pyridin-3-amine (0.075 g, 0.24 mmol), pyridazine-4-carboxylic acid (0.045 g, 0.36 mmol) and 2-chloro-1-methylpyridinium iodide (0.25 g, 0.96 mmol) was added anhydrous tetrahydrofuran (3.0 mL). The solution was heated at 65° C. for 5 min before N-ethyl-N-isopropylpropan-2-amine (0.31 g, 2.4 mmol) was added. The reaction mixture was stirred at 65° C. for 2 h. The reaction mixture was cooled to ambient temperature and diluted with ethyl acetate (200 mL). The reaction mixture was washed with saturated ammonium chloride solution (2×50 mL). The organic layer was dried over anhydrous magnesium sulfate, filtered and concentrated in vacuo. Purification of the residue by preparative HPLC, eluting with a gradient of 10 to 60% of acetonitrile in water containing 0.5% formic acid to provide the title compound as a colorless solid (0.0082 g, 8% yield): ¹H-NMR (500 MHz; DMSO-d₆) δ 10.52 (s, 1H), 9.46-9.43 (m, 1H), 9.33 (dd, J=2.2, 1.2 Hz, 1H), 8.26-8.23 (m, 1H), 7.84 (dd, J=5.3, 2.3 Hz, 1H), 7.34-7.29 (m, 1H), 7.25-7.20 (m, 1H), 7.18-7.13 (m, 1H), 6.86 (t, J=4.1 Hz, 1H), 3.97-3.83 (m, 2H), 3.83-3.67 (m, 2H), 2.49-2.40 (m, 2H); MS (ES+) m/z 418.0 (M+1).

### Example 140

### Synthesis of N-(2-(3,3-difluoropyrrolidin-1-yl)-4-(2-fluorophenyl)pyridin-3-yl)-2-isopropylpyrimidine-5-carboxamide

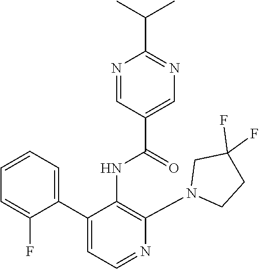 [SMILES: CC(C)c1ncc(C(=O)Nc2c(-c3ccccc3F)ccnc2N2CCC(F)(F)C2)cn1]

To a solution of 2-(3,3-difluoropyrrolidin-1-yl)-4-(2-fluorophenyl)pyridin-3-amine (0.67 g, 2.3 mmol), 2-isopropylpyrimidine-5-carboxylic acid (0.49 g, 3.0 mmol) and 2-chloro-1-methylpyridinium iodide (2.0 g, 7.9 mmol) was added anhydrous tetrahydrofuran (23 mL). The solution was heated at 65° C. for 5 min before N-ethyl-N-isopropylpropan-2-amine (2.9 g, 23 mmol) was added. The reaction mixture was stirred at 65° C. for 2.5 h. The reaction mixture was cooled to ambient temperature and diluted with ethyl acetate (200 mL). The reaction mixture was washed with saturated ammonium chloride solution (2×50 mL), 1M sodium hydroxide solution (50 mL), and saturated ammonium chloride solution (50 mL). The organic layer was dried over anhydrous magnesium sulfate, filtered and concentrated in vacuo. Purification of the residue by column chromatography, eluting with a gradient of 10 to 75% of ethyl acetate in heptane, provided a colorless solid. The solid was triturated with diethyl ether (10 mL) and filtered to provide the title compound as a colorless solid (0.55 g, 54% yield): ¹H-NMR (300 MHz; DMSO-d₆) δ 10.19 (s, 1H), 8.87 (s, 2H), 8.21 (d, J=4.9 Hz, 1H), 7.41-7.16 (m, 4H), 6.82 (d, J=4.9 Hz, 1H), 3.97-3.82 (m, 2H), 3.82-3.70 (m, 2H), 3.16 (dt, J=13.8, 6.9 Hz, 1H), 2.49-2.37 (m, 2H), 1.26 (d, J=6.9 Hz, 6H); MS (ES+) m/z 442.2 (M+1).

### Example 141

### Synthesis of N-(2-(3,3-difluoropyrrolidin-1-yl)-4-(2-fluorophenyl)pyridin-3-yl)-2-methylisonicotinamide

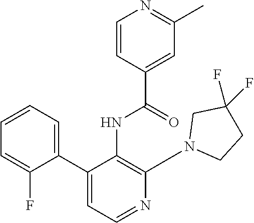 [SMILES: Cc1cc(C(=O)Nc2c(-c3ccccc3F)ccnc2N2CCC(F)(F)C2)ccn1]

To a solution of 2-(3,3-difluoropyrrolidin-1-yl)-4-(2-fluorophenyl)pyridin-3-amine (0.10 g, 0.34 mmol), 2-methylnicotinic acid (0.79 g, 0.58 mmol) and 2-chloro-1-methylpyridinium iodide (0.35 g, 1.4 mmol) was added anhydrous tetrahydrofuran (4.3 mL). The solution was heated at 65° C. for 5 min before N-ethyl-N-isopropylpropan-2-amine (0.44 g, 3.4 mmol) was added. The reaction mixture was stirred at 65° C. for 2 h. The reaction mixture was cooled to ambient temperature, diluted with ethyl acetate (150 mL), washed with saturated ammonium chloride solution (2×50 mL), dried over anhydrous magnesium sulfate, filtered and concentrated in vacuo. Purification of the residue by column chromatography, eluting with a gradient of 10 to 60% of ethyl acetate in heptane, provided the title compound as a colorless solid (0.14 g, 99% yield): ¹H-NMR (500 MHz; DMSO-d₆) δ 10.09 (s, 1H), 8.55 (d, J=5.1 Hz, 1H), 8.21 (d, J=4.9 Hz, 1H), 7.39-7.34 (m, 3H), 7.31-7.28 (m, 1H), 7.26-7.22 (m, 1H), 7.18 (td, J=7.5, 0.9 Hz, 1H), 6.81 (d, J=4.9 Hz, 1H), 3.92-3.83 (m, 2H), 3.75-3.71 (m, 2H), 2.50-2.49 (s, 3H), 2.49-2.40 (m, 2H); MS (ES+) m/z 413.2 (M+1).

### Example 142

### Synthesis of N-(4-(2,5-difluorophenyl)-2-(3,3-difluoropyrrolidin-1-yl)-6-methylpyridin-3-yl)-2-isopropylpyrimidine-5-carboxamide

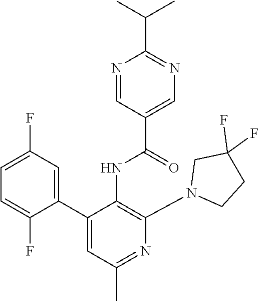 [SMILES: Cc1cc(-c2cc(F)ccc2F)c(NC(=O)c2cnc(C(C)C)nc2)c(N2CCC(F)(F)C2)n1]

### Step 1. Preparation of 2-chloro-4-(2,5-difluorophenyl)-6-methyl-3-nitropyridine

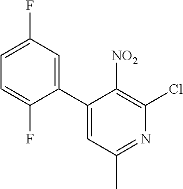 [SMILES: Cc1cc(-c2cc(F)ccc2F)c([N+](=O)[O-])c(Cl)n1]

A mixture of 2,4-dichloro-6-methyl-3-nitropyridine (1.5 g, 7.3 mmol), 1,4-dioxane (14 mL), and water (4.8 mL) was sparged with nitrogen for 10 min. The flask was added 2,5-difluorophenylboronic acid (1.2 g, 7.6 mmol), potassium carbonate (1.5 g, 11 mmol), and [1,1′-bis(diphenylphosphino)ferrocene]dichloropalladium(II), complex with dichloromethane (0.61 g, 0.72 mmol), and sparged with nitrogen for 2 min. The reaction mixture was stirred at 60° C. for 4 h. After cooling to ambient temperature the reaction mixture was diluted with ethyl acetate (300 mL), washed with saturated ammonium chloride (2×100 mL), dried over anhydrous magnesium sulfate, filtered, and concentrated in vacuo. Purification of the residue by column chromatography, eluting with 5-35% ethyl acetate in heptane, afforded the title compound as a slightly yellow solid (0.86 g, 42% yield): MS (ES+) m/z 285.0 (M+1), 287.0 (M+1).

### Step 2. Preparation of 4-(2,5-difluorophenyl)-2-(3,3-difluoropyrrolidin-1-yl)-6-methyl-3-nitropyridine

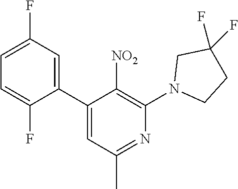 [SMILES: Cc1cc(-c2cc(F)ccc2F)c([N+](=O)[O-])c(N2CCC(F)(F)C2)n1]

To a solution of 2-chloro-4-(2,5-difluorophenyl)-6-methyl-3-nitropyridine (0.86 g, 3.0 mmol), anhydrous potassium carbonate (1.7 g, 12 mmol), and 3,3-difluoropyrrolidine hydrochloride (0.65 g, 4.5 mmol) was added N,N-dimethylformamide (10 mL). The reaction mixture was stirred at 45° C. for 18 h. The reaction mixture was diluted with ethyl acetate (200 mL), washed with saturated ammonium chloride (2×50 mL), dried over anhydrous magnesium sulfate, filtered, and concentrated in vacuo. Purification of the residue by column chromatography, eluting with 5-30% ethyl acetate in heptane, afforded the title compound as a green oil (0.78 g, 72% yield): MS (ES+) m/z356.2(M+1).

### Step 3. Preparation of 4-(2,5-difluorophenyl)-2-(3,3-difluoropyrrolidin-1-yl)-6-methylpyridin-3-amine

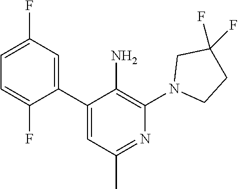 [SMILES: Cc1cc(-c2cc(F)ccc2F)c(N)c(N2CCC(F)(F)C2)n1]

To a solution of 4-(2,5-difluorophenyl)-2-(3,3-difluoropyrrolidin-1-yl)-6-methyl-3-nitropyridine (0.78 g, 2.2 mmol) was added anhydrous methanol (4.4 mL), ethyl acetate (4.4 mL), and 10% palladium on carbon (0.23 g). The reaction vessel was sealed and the reaction mixture was sparged with hydrogen gas for 5 min. The reaction mixture was stirred under a hydrogen atmosphere for 3 h. The reaction mixture was filtered through diatomaceous earth (i.e., Celite®), washed with ethyl acetate (5×20 mL) and concentrated in vacuo. The residue was used as is (0.70 g, 98% yield): MS (ES+) m/z 326.2 (M+1).

### Step 4. Preparation of N-(4-(2,5-difluorophenyl)-2-(3,3-difluoropyrrolidin-1-yl)-6-methylpyridin-3-yl)-2-isopropylpyrimidine-5-carboxamide

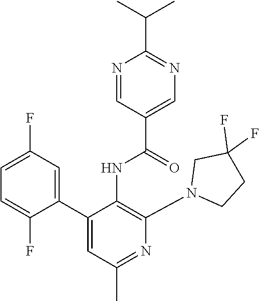 [SMILES: Cc1cc(-c2cc(F)ccc2F)c(NC(=O)c2cnc(C(C)C)nc2)c(N2CCC(F)(F)C2)n1]

To a solution of 4-(2,5-difluorophenyl)-2-(3,3-difluoropyrrolidin-1-yl)-6-methylpyridin-3-amine (0.10 g, 0.31 mmol), 1-isopropylpyrimidine-5-carboxylic acid (0.092 g, 0.55 mmol) and 2-chloro-1-methylpyridinium iodide (0.31 g, 1.2 mmol) was added anhydrous tetrahydrofuran (3.8 mL). The solution was heated at 65° C. for 2 min before N-ethyl-N-isopropylpropan-2-amine (0.40 g, 3.1 mmol) was added. The reaction mixture was stirred at 65° C. for 1 h. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue column chromatography, eluting with a gradient of 12 to 70% of ethyl acetate in heptane, to provide the title compound as a colorless solid (0.068 g, 44% yield): ¹H-NMR (500 MHz; DMSO-d₆) δ 10.12 (s, 1H), 8.91 (s, 2H), 7.33-7.29 (m, 1H), 7.22 (td, J=8.0, 4.3 Hz, 1H), 7.15-7.11 (m, 1H), 6.71 (s, 1H), 3.92-3.85 (m, 2H), 3.77-3.71 (m, 2H), 3.18 (dt, J=13.8, 6.9 Hz, 1H), 2.47-2.38 (m, 5H), 1.27 (d, J=6.9 Hz, 6H); MS (ES+) m/z 474.2 (M+1).

### Example 143

### Synthesis of N-(4-(2,5-difluorophenyl)-2-(3,3-difluoropyrrolidin-1-yl)-6-methylpyridin-3-yl)-1-isopropyl-1H-pyrazole-4-carboxamide

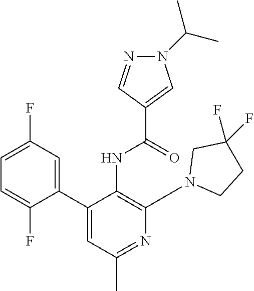 [SMILES: Cc1cc(-c2cc(F)ccc2F)c(NC(=O)c2cnn(C(C)C)c2)c(N2CCC(F)(F)C2)n1]

To a solution of 4-(2,5-difluorophenyl)-2-(3,3-difluoropyrrolidin-1-yl)-6-methylpyridin-3-amine (0.10 g, 0.31 mmol), 1-isopropyl-1H-pyrazole-4-carboxylic acid (0.095 g, 0.61 mmol) and 2-chloro-1-methylpyridinium iodide (0.31 g, 1.2 mmol) was added anhydrous tetrahydrofuran (3.8 mL). The solution was heated at 65° C. for 2 min before N-ethyl-N-isopropylpropan-2-amine (0.40 g, 3.1 mmol) was added. The reaction mixture was stirred at 65° C. for 1 h after which the reaction mixture was added 1-isopropyl-1H-pyrazole-4-carboxylic acid (0.050 g, 0.29 mmol) and stirred for 4 h. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue by preparative HPLC, eluting with a gradient of 10 to 80% of acetonitrile in water with 0.5% formic acid to provide the title compound as a colorless solid (0.060 g, 41% yield): ¹H-NMR (500 MHz; DMSO-d₆) δ 9.34 (s, 1H), 8.16 (s, 1H), 7.83 (s, 1H), 7.29-7.25 (m, 1H), 7.21-7.17 (m, 1H), 7.17-7.09 (m, 1H), 6.65 (s, 1H), 4.50-4.45 (m, 1H), 3.91-3.81 (m, 2H), 3.76-3.69 (m, 2H), 2.46-2.37 (m, 5H), 1.41-1.37 (m, 6H); MS (ES+) m/z 462.2 (M+1).

### Example 144

### Synthesis of (1r,4r)-N-(4-(2,5-difluorophenyl)-2-(3,3-difluoropyrrolidin-1-yl)pyridin-3-yl)-4-methoxycyclohexane-1-carboxamide

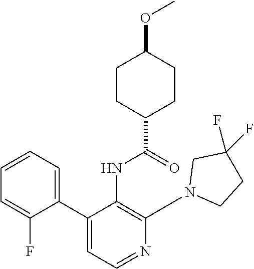

To a solution of 2-(3,3-difluoropyrrolidin-1-yl)-4-(2-fluorophenyl)pyridin-3-amine (0.10 g, 0.34 mmol), (1r,4r)-4-methoxycyclohexane-1-carboxylic acid (0.092 g, 0.58 mmol) and 2-chloro-1-methylpyridinium iodide (0.35 g, 1.4 mmol) was added anhydrous tetrahydrofuran (4.3 mL). The solution was heated at 65° C. for 2 min before N-ethyl-N-isopropylpropan-2-amine (0.44 g, 3.4 mmol) was added. The reaction mixture was stirred at 65° C. for 20 h. The reaction mixture was cooled to ambient temperature, diluted with ethyl acetate (150 mL), washed with saturated ammonium chloride solution (2×50 mL), dried over anhydrous magnesium sulfate, filtered and concentrated in vacuo. Purification of the residue by column chromatography, eluting with a gradient of 10 to 100% of ethyl acetate in heptane, followed by trituration with acetonitrile (10 mL), provided the title compound as a colorless solid (0.056 g, 38% yield): ¹H-NMR (500 MHz; DMSO-d₆) δ 9.23 (s, 1H), 8.12 (d, J=4.9 Hz, 1H), 7.45-7.40 (m, 1H), 7.28-7.17 (m, 3H), 6.71 (d, J=4.9 Hz, 1H), 3.92-3.81 (m, 2H), 3.78-3.69 (m, 2H), 3.19 (s, 3H), 2.97-2.91 (m, 1H), 2.50-2.41 (m, 2H), 2.06-2.00 (m, 1H), 1.90-1.88 (m, 2H), 1.40-1.33 (m, 2H), 1.11-0.92 (m, 4H); MS (ES+) m/z 434.2 (M+1).

### Example 145

### Synthesis of N-[2-(3,3-difluoropyrrolidin-1-yl)-4-(2-fluorophenyl)-3-pyridyl]-2-methyl-thiazole-5-carboxamide

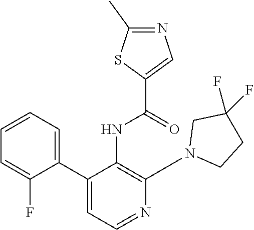 [SMILES: Cc1ncc(C(=O)Nc2c(-c3ccccc3F)ccnc2N2CCC(F)(F)C2)s1]

To a solution of 2-(3,3-difluoropyrrolidin-1-yl)-4-(2-fluorophenyl)pyridin-3-amine (0.10 g, 0.34 mmol), 2-methyl-1,3-thiazole-5-carboxylic acid (0.073 g, 0.51 mmol) and 2-chloro-1-methylpyridinium iodide (0.35 g, 1.4 mmol) was added anhydrous tetrahydrofuran (4.3 mL). The solution was heated at 65° C. for 5 min before N-ethyl-N-isopropylpropan-2-amine (0.44 g, 3.4 mmol) was added. The reaction mixture was stirred at 65° C. for 20 h. The reaction mixture was cooled to ambient temperature, diluted with ethyl acetate (150 mL), washed with saturated ammonium chloride solution (2×50 mL), dried over anhydrous magnesium sulfate, filtered and concentrated in vacuo. Purification of the residue by column chromatography, eluting with a gradient of 15 to 100% of ethyl acetate in heptane, provided the title compound as a pale yellow solid (0.10 g, 69% yield): ¹H-NMR (500 MHz; DMSO-d₆) δ 9.96 (s, 1H), 8.19 (d, J=5.0 Hz, 1H), 8.13 (s, 1H), 7.39-7.34 (m, 1H), 7.29 (td, J=7.6, 1.5 Hz, 1H), 7.25-7.21 (m, 1H), 7.17 (td, J=7.5, 1.1 Hz, 1H), 6.80 (d, J=4.8 Hz, 1H), 3.93-3.72 (m, 4H), 2.64 (s, 3H), 2.49-2.41 (m, 2H); MS (ES+) m/z 419.0 (M+1).

### Example 146

### Synthesis of 2-chloro-N-[2-(3,3-difluoropyrrolidin-1-yl)-4-(2-fluorophenyl)-3-pyridyl]pyrimidine-5-carboxamide

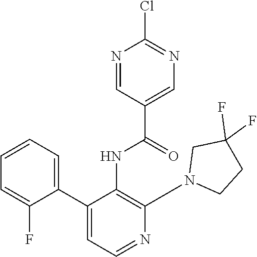 [SMILES: O=C(Nc1c(-c2ccccc2F)ccnc1N1CCC(F)(F)C1)c1cnc(Cl)nc1]

To a solution of 2-(3,3-difluoropyrrolidin-1-yl)-4-(2-fluorophenyl)pyridin-3-amine (0.43 g, 1.5 mmol), 2-chloropyrimidine-5-carboxylic acid (0.35 g, 2.2 mmol) and 2-chloro-1-methylpyridinium iodide (1.3 g, 5.1 mmol) was added anhydrous tetrahydrofuran (18 mL). The solution was heated at 65° C. for 5 min before N-ethyl-N-isopropylpropan-2-amine (1.9 g, 15 mmol) was added. The reaction mixture was stirred at 65° C. for 3 h. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification of the residue by column chromatography, eluting with a gradient of 10 to 60% of ethyl acetate in heptane, provided the title compound as a colorless solid (0.23 g, 35% yield): ¹H-NMR (500 MHz; DMSO-d₆) δ 10.32 (s, 1H), 8.90 (dd, J=10.4, 3.3 Hz, 2H), 8.22 (d, J=4.9 Hz, 1H), 7.40-7.35 (m, 1H), 7.32-7.28 (m, 1H), 7.27-7.23 (m, 1H), 7.21-7.18 (m, 1H), 6.83 (d, J=4.9 Hz, 1H), 3.96-3.82 (m, 2H), 3.82-3.70 (m, 2H), 2.48-2.40 (m, 2H); MS (ES+) m/z 434.0 (M+1), 436.0 (M+1).

### Example 147

### Synthesis of N-[6-chloro-2-(3,3-difluoropyrrolidin-1-yl)-4-phenyl-3-pyridyl]-2-isopropyl-pyrimidine-5-carboxamide

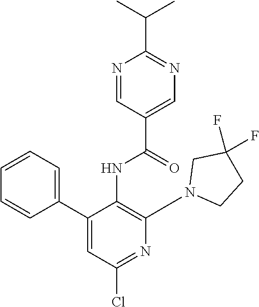 [SMILES: CC(C)c1ncc(C(=O)Nc2c(-c3ccccc3)cc(Cl)nc2N2CCC(F)(F)C2)cn1]

### Step 1. Preparation of 2,6-dichloro-3-nitro-4-phenyl-pyridine

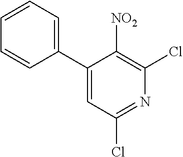 [SMILES: O=[N+]([O-])c1c(-c2ccccc2)cc(Cl)nc1Cl]

A mixture of 2,4,6-trichloro-3-nitropyridine (1.0 g, 4.4 mmol), 1,4-dioxane (8.5 mL), and water (2.9 mL) was sparged with nitrogen for 10 min. To the mixture was added phenylboronic acid (0.54 g, 4.4 mmol), potassium carbonate (0.91 g, 6.6 mmol), and [1,1′-bis(diphenylphosphino)ferrocene]dichloropalladium(II), complex with dichloromethane (0.37 g, 0.44 mmol), and the mixture was sparged with nitrogen for 2 min. The reaction mixture was stirred at 50° C. for 1 h. After cooling to ambient temperature the reaction mixture was diluted with ethyl acetate (230 mL), washed with saturated ammonium chloride (50 mL), dried over anhydrous magnesium sulfate, filtered, and concentrated in vacuo. Purification of the residue by column chromatography, eluting with 0-20% ethyl acetate in heptane, afforded the title compound as a slightly yellow solid (0.86 g, 73% yield): MS (ES+) m/z 269.0 (M+1), 271.0 (M+1), 273.0 (M+1).

### Step 2. Preparation of 6-chloro-2-(3,3-difluoropyrrolidin-1-yl)-3-nitro-4-phenyl-pyridine

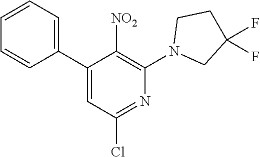 [SMILES: O=[N+]([O-])c1c(-c2ccccc2)cc(Cl)nc1N1CCC(F)(F)C1]

To a solution of 2,6-dichloro-3-nitro-4-phenyl-pyridine (0.86 g, 3.2 mmol), was added N,N-dimethylformamide (11 mL). The solution was cooled to −78° C. and anhydrous potassium carbonate (1.3 g, 9.6 mmol), and 3,3-difluoropyrrolidine hydrochloride (0.69 g, 4.8 mmol) were added. The reaction mixture was allowed to warm to ambient temperature and stir for 90 min. The reaction mixture was diluted with ethyl acetate (250 mL), washed with saturated ammonium chloride (2×50 mL), dried over anhydrous magnesium sulfate, filtered, and concentrated in vacuo. Purification of the residue by column chromatography, eluting with 5-30% ethyl acetate in heptane, afforded the title compound a yellow oil that was a mixture of addition adducts (0.74 g, 34% yield): MS (ES+) m/z 340.2 (M+1), 342.2 (M+1).

### Step 3. Preparation of 6-chloro-2-(3,3-difluoropyrrolidin-1-yl)-4-phenyl-pyridin-3-amine

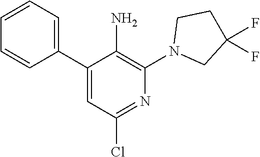 [SMILES: Nc1c(-c2ccccc2)cc(Cl)nc1N1CCC(F)(F)C1]

To a solution of 6-chloro-2-(3,3-difluoropyrrolidin-1-yl)-3-nitro-4-phenyl-pyridine (0.74 g, 2.2 mmol) was added anhydrous ethanol (4.4 mL) and water (4.4 mL). Ammonium chloride (1.2 g, 22 mmol) and iron powder (1.2 g, 22 mmol) was added to the reaction mixture. A reflux condenser was added and the solution was heated to reflux for 24 h. After cooling to ambient temperature, the reaction mixture diluted with ethyl acetate (200 mL) and sonicated for 5 min. The mixture was filtered through diatomaceous earth (i.e., Celite®), washing with ethyl acetate (3×20 mL). The combined organic layers were washed with saturated sodium bicarbonate (50 mL), saturated potassium carbonate (50 mL), dried over anhydrous magnesium sulfate, filtered, and concentrated in vacuo. Purification of the residue by column chromatography, eluting with 10-50% ethyl acetate in heptane, afforded the title compound as a light brown oil (0.41 g, 60% yield): MS (ES+) m/z 310.2 (M+1), 312.2 (M+1).

### Step 4. Preparation of N-[6-chloro-2-(3,3-difluoropyrrolidin-1-yl)-4-phenyl-3-pyridyl]-2-isopropyl-pyrimidine-5-carboxamide

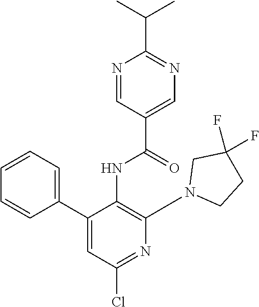 [SMILES: CC(C)c1ncc(C(=O)Nc2c(-c3ccccc3)cc(Cl)nc2N2CCC(F)(F)C2)cn1]

To a solution of 6-chloro-2-(3,3-difluoropyrrolidin-1-yl)-4-phenyl-pyridin-3-amine (0.10 g, 0.32 mmol), 1-isopropylpyrimidine-5-carboxylic acid (0.080 g, 0.48 mmol) and 2-chloro-1-methylpyridinium iodide (0.33 g, 1.3 mmol) was added anhydrous tetrahydrofuran (3.2 mL). The solution was heated at 65° C. for 2 min before N-ethyl-N-isopropylpropan-2-amine (0.33 g, 2.6 mmol) was added. The reaction mixture was stirred at 65° C. for 18 h. The reaction mixture was cooled to ambient temperature, diluted with methanol (5 mL) and 10 M sodium hydroxide solution (2 mL). The solution was stirred at ambient temperature for 20 min. The reaction mixture was diluted with ethyl acetate (150 mL), washed with saturated ammonium chloride (2×50 mL), dried over anhydrous magnesium sulfate, filtered, and concentrated in vacuo. Purification of the residue column chromatography, eluting with a gradient of 15 to 100% of ethyl acetate in heptane, to provide the title compound as a colorless solid (0.033 g, 21% yield): ¹H-NMR (500 MHz; DMSO-d₆) δ 10.32 (s, 1H), 8.90 (s, 2H), 8.22 (d, J=4.9 Hz, 1H), 7.39-7.18 (m, 4H), 6.83 (d, J=4.9 Hz, 1H), 3.92-3.73 (m, 4H), 2.50-2.40 (m, 3H), 1.26 (d, J=6.9 Hz, 6H); MS (ES+) m/z 458.2 (M+1), 460.2 (M+1).

### Example 148

### Synthesis of N-[6-chloro-2-(3,3-difluoropyrrolidin-1-yl)-4-phenyl-3-pyridyl]-1-isopropyl-pyrazole-4-carboxamide

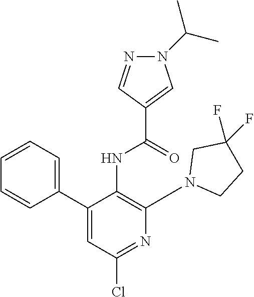 [SMILES: CC(C)n1cc(C(=O)Nc2c(-c3ccccc3)cc(Cl)nc2N2CCC(F)(F)C2)cn1]

To a solution of 6-chloro-2-(3,3-difluoropyrrolidin-1-yl)-4-phenyl-pyridin-3-amine (0.10 g, 0.32 mmol), 1-isopropylpyrimidine-5-carboxylic acid (0.075 g, 0.48 mmol) and 2-chloro-1-methylpyridinium iodide (0.33 g, 1.3 mmol) was added anhydrous tetrahydrofuran (3.2 mL). The solution was heated at 65° C. for 2 min before N-ethyl-N-isopropylpropan-2-amine (0.33 g, 2.6 mmol) was added. The reaction mixture was stirred at 65° C. for 18 h. The reaction mixture was cooled to ambient temperature, diluted with methanol (5 mL) and 10 M sodium hydroxide solution (2 mL). The solution was stirred at ambient temperature for 20 min. The reaction mixture was diluted with ethyl acetate (150 mL), washed with saturated ammonium chloride (2×50 mL), dried over anhydrous magnesium sulfate, filtered, and concentrated in vacuo. Purification of the residue by preparative HPLC eluting with a gradient of 10 to 80% of acetonitrile in water containing 0.5% formic acid to provide the title compound as a colorless solid (0.026 g, 18% yield): ¹H-NMR (500 MHz; DMSO-d₆) δ 9.40 (s, 1H), 8.15 (s, 1H), 7.83 (d, J=0.4 Hz, 1H), 7.41-7.33 (m, 5H), 6.80 (s, 1H), 4.49 (quintet, J=6.7 Hz, 1H), 3.81-3.79 (m, 4H), 2.45-2.41 (m, 2H), 1.39 (d, J=6.7 Hz, 6H); MS (ES+) m/z 446.2 (M+1), 448.2 (M+1).

### Example 149

### Synthesis of N-[2-(3,3-difluoropyrrolidin-1-yl)-4-(2-fluorophenyl)-3-pyridyl]-2-morpholino-pyrimidine-5-carboxamide

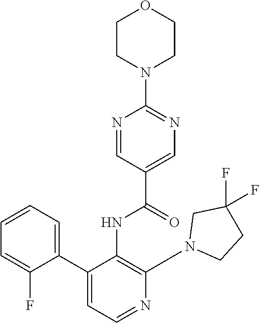 [SMILES: O=C(Nc1c(-c2ccccc2F)ccnc1N1CCC(F)(F)C1)c1cnc(N2CCOCC2)nc1]

To a solution of 2-chloro-N-[2-(3,3-difluoropyrrolidin-1-yl)-4-(2-fluorophenyl)-3-pyridyl]pyrimidine-5-carboxamide (0.60 g, 0.14 mmol) was added anhydrous N,N-dimethylformamide (1.4 mL). Morpholine (0.031 g, 0.69 mmol) and 60% sodium hydride dispersion in mineral oil (0.011 g, 0.28 mmol) was added. The reaction mixture was stirred at ambient temperature for 1 h. The reaction mixture was diluted with ethyl acetate (25 mL), washed with saturated ammonium chloride solution (10 mL), dried over anhydrous magnesium sulfate, filtered and concentrated in vacuo. Purification of the residue by column chromatography, eluting with 13-100% ethyl acetate in heptane, provided the title compound as a colorless solid (0.036 g, 53% yield): ¹H-NMR (300 MHz; DMSO-d₆) δ 9.74 (s, 1H), 8.64 (s, 2H), 8.18 (d, J=5.0 Hz, 1H), 7.39-7.14 (m, 4H), 6.79 (dd, J=5.0, 0.8 Hz, 1H), 3.94-3.80 (m, 2H), 3.81-3.68 (m, 6H), 3.66-3.61 (m, 4H), 2.49-2.36 (m, 2H); MS (ES+) m/z 485.2 (M+1).

### Example 150

### Synthesis of N-[2-(3,3-difluoropyrrolidin-1-yl)-4-(2-fluorophenyl)-3-pyridyl]-2-methoxy-pyrimidine-5-carboxamide

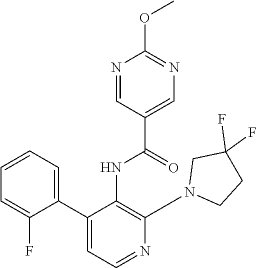 [SMILES: COc1ncc(C(=O)Nc2c(-c3ccccc3F)ccnc2N2CCC(F)(F)C2)cn1]

To a solution of 2-chloro-N-[2-(3,3-difluoropyrrolidin-1-yl)-4-(2-fluorophenyl)-3-pyridyl]pyrimidine-5-carboxamide (0.60 g, 0.14 mmol) was added anhydrous N,N-dimethylformamide (1.4 mL). Anhydrous methanol (0.022 g, 0.69 mmol) and 60% sodium hydride dispersion in mineral oil (0.011 g, 0.28 mmol) was added. The reaction mixture was stirred at ambient temperature for 1 h. The reaction mixture was diluted with ethyl acetate (25 mL), washed with saturated ammonium chloride solution (10 mL), dried over anhydrous magnesium sulfate, filtered and concentrated in vacuo. Purification of the residue by column chromatography, eluting with 13-100% ethyl acetate in heptane, provided the title compound as a colorless solid (0.019 g, 32% yield): ¹H-NMR (300 MHz; DMSO-d₆) δ 10.06 (s, 1H), 8.80 (d, J=2.7 Hz, 2H), 8.21 (d, J=5.0 Hz, 1H), 7.39-7.15 (m, 4H), 6.81 (dd, J=5.0, 0.8 Hz, 1H), 3.96-3.93 (m, 3H), 3.93-3.71 (m, 4H), 2.49-2.39 (m, 2H); MS (ES+) m/z 430.2 (M+1).

### Example 151

### Synthesis of N-[2-(3,3-difluoropyrrolidin-1-yl)-4-(2-fluorophenyl)-3-pyridyl]-2-isopropoxy-pyrimidine-5-carboxamide

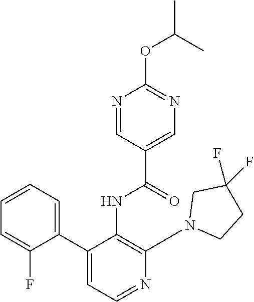 [SMILES: CC(C)Oc1ncc(C(=O)Nc2c(-c3ccccc3F)ccnc2N2CCC(F)(F)C2)cn1]

A vial containing 2-chloro-N-[2-(3,3-difluoropyrrolidin-1-yl)-4-(2-fluorophenyl)-3-pyridyl]pyrimidine-5-carboxamide (0.60 g, 0.14 mmol) was added anhydrous N,N-dimethylformamide (1.4 mL). Isopropanol (0.084 g, 1.4 mmol) and 60% sodium hydride dispersion in mineral oil (0.011 g, 0.28 mmol) was added. The reaction vial was sealed and heated to 50° C. for 1 h. The reaction mixture was diluted with ethyl acetate (25 mL), washed with saturated ammonium chloride solution (10 mL), dried over anhydrous magnesium sulfate, filtered and concentrated in vacuo. Purification by preparative HPLC, eluting with a gradient of 10 to 80% of acetonitrile in water containing 0.5% formic acid, provided the title compound as a colorless solid (0.009 g, 15% yield): ¹H-NMR (300 MHz; DMSO-d₆) δ 10.02 (s, 1H), 8.78-8.76 (m, 2H), 8.20 (d, J=5.0 Hz, 1H), 7.40-7.15 (m, 4H), 6.81 (dd, J=5.0, 0.7 Hz, 1H), 5.23 (quintet, J=6.2 Hz, 1H), 3.95-3.69 (m, 4H), 2.49-2.36 (m, 2H), 1.31 (t, J=5.9 Hz, 6H); MS (ES+) m/z 458.2 (M+1).

### Example 152

### Synthesis of N-[2-(3,3-difluoropyrrolidin-1-yl)-4-(2-fluorophenyl)-3-pyridyl]-2-(dimethylamino)pyrimidine-5-carboxamide

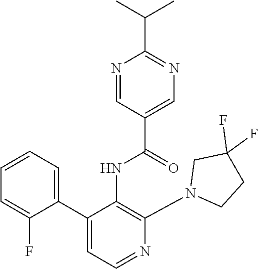 [SMILES: CC(C)c1ncc(C(=O)Nc2c(-c3ccccc3F)ccnc2N2CCC(F)(F)C2)cn1]

To 2-chloro-N-[2-(3,3-difluoropyrrolidin-1-yl)-4-(2-fluorophenyl)-3-pyridyl]pyrimidine-5-carboxamide (0.60 g, 0.14 mmol) was added anhydrous N,N-dimethylformamide (1.4 mL). To the mixture was added dimethylamino hydrochloride (0.042 g, 69 mmol) and 60% sodium hydride dispersion in mineral oil (0.011 g, 0.28 mmol). The reaction mixture was stirred at ambient temperature for 1 h. The reaction mixture was diluted with ethyl acetate (25 mL), washed with saturated ammonium chloride solution (10 mL), dried over anhydrous magnesium sulfate, filtered and concentrated in vacuo. Purification of the residue by column chromatography, eluting with 13-100% ethyl acetate in heptane, provided the title compound as a colorless solid (0.041 g, 67% yield): ¹H-NMR (300 MHz; DMSO-d₆) δ 9.68 (s, 1H), 8.58 (s, 2H), 8.18 (d, J=5.0 Hz, 1H), 7.36-7.13 (m, 4H), 6.79 (dd, J=5.0, 0.8 Hz, 1H), 3.88-3.72 (m, 4H), 3.14 (s, 6H), 2.44 (td, J=13.5, 6.4 Hz, 2H); MS (ES+) m/z 443.2 (M+1).

### Example 153

### Synthesis of N-[2-(3,3-difluoropyrrolidin-1-yl)-4-(2-fluorophenyl)-3-pyridyl]-2-hydroxy-pyrimidine-5-carboxamide

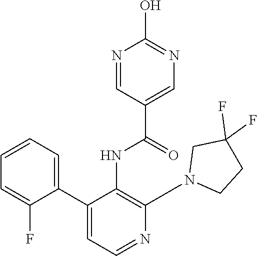 [SMILES: O=C(Nc1c(-c2ccccc2F)ccnc1N1CCC(F)(F)C1)c1cnc(O)nc1]

To 2-chloro-N-[2-(3,3-difluoropyrrolidin-1-yl)-4-(2-fluorophenyl)-3-pyridyl]pyrimidine-5-carboxamide (0.60 g, 0.14 mmol) was added anhydrous N,N-dimethylformamide (1.4 mL). Anhydrous isopropanol (0.084 g, 1.4 mmol) and 60% sodium hydride dispersion in mineral oil (0.011 g, 0.28 mmol) were added. The reaction vial was sealed and heated to 50° C. for 1 h. The reaction mixture was diluted with ethyl acetate (25 mL), washed with saturated ammonium chloride solution (10 mL), dried over anhydrous magnesium sulfate, filtered and concentrated in vacuo.

Purification by preparative HPLC, eluting with a gradient of 10 to 80% of acetonitrile in water containing 0.5% formic acid, provided the title compound as a colorless solid (0.006 g, 10% yield): ¹H-NMR (300 MHz; DMSO-d₆) δ 9.68 (s, 1H), 8.70 (br s, 1H), 8.37-8.32 (m, 1H), 8.17-8.14 (m, 1H), 7.82 (br s, 1H), 7.44-7.35 (m, 1H), 7.27-7.20 (m, 1H), 7.18-7.06 (m, 2H), 6.90-6.85 (m, 1H), 3.76-3.66 (m, 2H), 3.59-3.44 (m, 2H), 2.48-2.36 (m, 2H); MS (ES+) m/z 416.2 (M+1).

### Example 154

### Synthesis of N-[2-(3,3-difluoropyrrolidin-1-yl)-4-(2-fluorophenyl)-3-pyridyl]-3-methyl-isothiazole-5-carboxamide

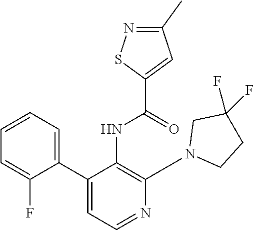 [SMILES: Cc1cc(C(=O)Nc2c(-c3ccccc3F)ccnc2N2CCC(F)(F)C2)sn1]

To a solution of 2-(3,3-difluoropyrrolidin-1-yl)-4-(2-fluorophenyl)pyridin-3-amine (0.060 g, 0.20 mmol), 3-methyl-1,2-thiazole-5-carboxylic acid (0.044 g, 0.31 mmol) and 2-chloro-1-methylpyridinium iodide (0.18 g, 0.71 mmol) was added anhydrous tetrahydrofuran (2.6 mL). The solution was heated at 65° C. for 2 min before N-ethyl-N-isopropylpropan-2-amine (0.26 g, 0.36 mmol) was added. The reaction mixture was stirred at 65° C. for 3 h. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification of the residue by column chromatography, eluting with a gradient of 10 to 60% of ethyl acetate in heptane, provided the title compound as a colorless solid (0.040 g, 47% yield): ¹H-NMR (500 MHz; DMSO-d₆) δ 10.20 (s, 1H), 8.21 (d, J=5.0 Hz, 1H), 7.61 (s, 1H), 7.37 (dddd, J=8.3, 7.2, 5.3, 1.9 Hz, 1H), 7.31-7.23 (m, 2H), 7.20-7.15 (m, 1H), 6.81 (dd, J=5.0, 0.9 Hz, 1H), 3.93-3.83 (m, 2H), 3.79-3.71 (m, 2H), 2.47-2.38 (m, 5H); MS (ES+) m/z 419.2 (M+1).

### Example 155

### Synthesis of N-[2-(3,3-difluoropyrrolidin-1-yl)-4-(2-fluorophenyl)-3-pyridyl]-6-methoxy-pyridine-3-carboxamide

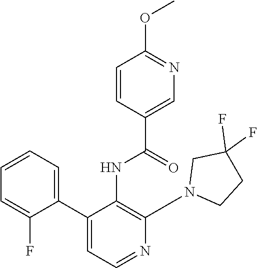 [SMILES: COc1ccc(C(=O)Nc2c(-c3ccccc3F)ccnc2N2CCC(F)(F)C2)cn1]

To a solution of 2-(3,3-difluoropyrrolidin-1-yl)-4-(2-fluorophenyl)pyridin-3-amine (0.10 g, 0.34 mmol), 6-methoxynicotinic acid (0.078 g, 0.51 mmol) and 2-chloro-1-methylpyridinium iodide (0.30 g, 1.2 mmol) was added anhydrous tetrahydrofuran (4.2 mL). The solution was heated at 65° C. for 2 min before N-ethyl-N-isopropylpropan-2-amine (0.44 g, 3.4 mmol) was added. The reaction mixture was stirred at 65° C. for 18 h. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification by preparative HPLC, eluting with a gradient of 10 to 60% of acetonitrile in water containing 0.5% formic acid, provided the title compound as a colorless solid (0.027 g, 18% yield): ¹H-NMR (300 MHz; DMSO-d₆) δ 9.87 (s, 1H), 8.49 (dd, J=2.5, 0.6 Hz, 1H), 8.19 (d, J=5.0 Hz, 1H), 7.94 (dd, J=8.7, 2.5 Hz, 1H), 7.38-7.22 (m, 3H), 7.19-7.13 (m, 1H), 6.85 (dd, J=8.7, 0.6 Hz, 1H), 6.79 (dd, J=5.0, 0.8 Hz, 1H), 3.95-3.82 (m, 5H), 3.80-3.70 (m, 2H), 2.48-2.36 (m, 2H); MS (ES+) m/z 429.2 (M+1).

### Example 156

### Synthesis of N-[2-(3,3-difluoropyrrolidin-1-yl)-6-methoxy-4-phenyl-3-pyridyl]-2-isopropyl-pyrimidine-5-carboxamide

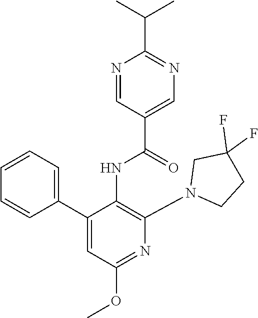 [SMILES: COc1cc(-c2ccccc2)c(NC(=O)c2cnc(C(C)C)nc2)c(N2CCC(F)(F)C2)n1]

### Step 1. Preparation of 2-(3,3-difluoropyrrolidin-1-yl)-6-methoxy-3-nitro-4-phenyl-pyridine

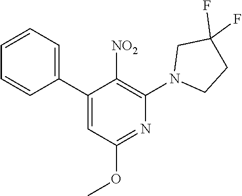 [SMILES: COc1cc(-c2ccccc2)c([N+](=O)[O-])c(N2CCC(F)(F)C2)n1]

To a solution of 6-chloro-2-(3,3-difluoropyrrolidin-1-yl)-3-nitro-4-phenyl-pyridine (0.60 g, 1.8 mmol) was added anhydrous N,N-dimethylformamide (5.8 mL) and anhydrous methanol (1.1 g, 35 mmol). Solid 60% dispersion of sodium hydride in mineral oil (0.14 g, 3.5 mmol) was added and the solution was stirred at ambient temperature for 1 h. After 1 h anhydrous methanol (3.0 mL) and 60% dispersion of sodium hydride in mineral oil (0.14 g, 3.5 mmol) was added and the reaction mixture was heated to 50° C. for 1 h. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification by column chromatography, eluting with 5 to 75% ethyl acetate in heptane, provided the title compound as a yellow oil (0.56 g, 95% yield): MS (ES+) m/z 337.2 (M+1).

### Step 2. Preparation of 2-(3,3-difluoropyrrolidin-1-yl)-6-methoxy-4-phenyl-pyridin-3-amine

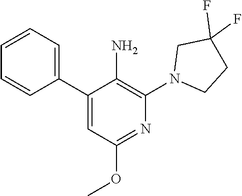 [SMILES: COc1cc(-c2ccccc2)c(N)c(N2CCC(F)(F)C2)n1]

To a solution of 2-(3,3-difluoropyrrolidin-1-yl)-6-methoxy-3-nitro-4-phenyl-pyridine (0.56 g, 1.7 mmol) was added anhydrous methanol (3.3 mL) and ethyl acetate (3.3 mL). Solid ammonium formate (1.1 g, 17 mmol) and 10% palladium on carbon (0.59 g) was added and the reaction mixture was heated to reflux for 1 h. A further ammonium formate (1.1 g, 17 mmol) and 10% palladium on carbon (0.59 g) was added and the reaction mixture was heated to reflux for 1 h. The reaction mixture was cooled to ambient temperature, diluted with ethyl acetate (50 mL), filtered through a pad of diatomaceous earth (i.e., Celite®), washing the residue with ethyl acetate (3×20 mL).

The combined organics were dried over anhydrous magnesium sulfate, filtered and concentrated in vacuo. Purification by column chromatography, eluting with 3 to 20% ethyl acetate in heptane, provided the title compound as a colorless oil (0.38 g, 75% yield): MS (ES+) m/z 306.2 (M+1).

### Step 3. Preparation of N-[2-(3,3-difluoropyrrolidin-1-yl)-6-methoxy-4-phenyl-3-pyridyl]-2-isopropyl-pyrimidine-5-carboxamide

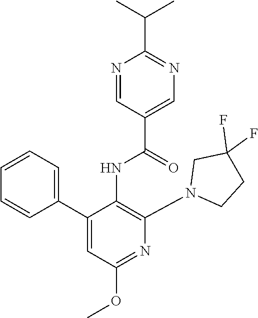 [SMILES: COc1cc(-c2ccccc2)c(NC(=O)c2cnc(C(C)C)nc2)c(N2CCC(F)(F)C2)n1]

To a solution of 2-(3,3-difluoropyrrolidin-1-yl)-6-methoxy-4-phenyl-pyridin-3-amine (0.38 g, 1.2 mmol), 2-isopropylpyrimidine-5-carboxylic acid (0.23 g, 1.4 mmol), and 2-chloro-1-methylpyridinium iodide (0.95 g, 3.7 mmol) was added anhydrous tetrahydrofuran (12 mL). A reflux condenser was added, the solution was heated to reflux and N-ethyl-N-isopropylpropan-2-amine (1.6 g, 12 mmol) was added after 1 min. The reaction mixture was heated to reflux for 45 min. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification by column chromatography, eluting with 10 to 100% ethyl acetate in heptane, provided the title compound as a mixture of compounds (0.44 g, 50% purity, 39% yield): MS (ES+) m/z 454.2 (M+1).

### Example 157

### Synthesis of N-[2-(3,3-difluoropyrrolidin-1-yl)-6-methoxy-4-phenyl-3-pyridyl]-2-isopropyl-pyrimidine-5-carboxamide

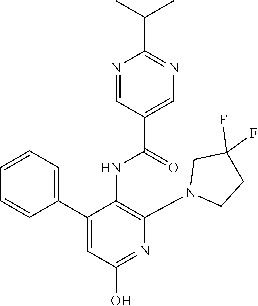 [SMILES: CC(C)c1ncc(C(=O)Nc2c(-c3ccccc3)cc(O)nc2N2CCC(F)(F)C2)cn1]

To a solution of N-[2-(3,3-difluoropyrrolidin-1-yl)-6-methoxy-4-phenyl-3-pyridyl]-2-isopropyl-pyrimidine-5-carboxamide (0.44 g, 0.49 mmol) was added anhydrous N,N-dimethylformamide (5.0 mL). Anhydrous lithium chloride (0.10 g, 2.5 mmol) and β-toluenesulfonic acid monohydrate (0.47 g, 2.5 mmol) was added, the flask was sealed and heated to 120° C. for 18 h. The reaction mixture was cooled to ambient temperature and anhydrous lithium chloride (0.21 g, 5 mmol) and p-toluenesulfonic acid monohydrate (0.94 g, 5 mmol) was added. The flask was sealed and heated to 120° C. for a further 4 h. The reaction mixture was cooled to ambient temperature, diluted with ethyl acetate (250 mL), washed with saturated ammonium chloride solution (2×50 mL), dried over anhydrous magnesium sulfate, filtered and concentrated in vacuo. Purification by preparative HPLC, eluting with a gradient of 30 to 95% of acetonitrile in water containing 0.5% formic acid, provided the title compound as a colorless solids: N-[2-(3,3-difluoropyrrolidin-1-yl)-6-hydroxy-4-phenyl-3-pyridyl]-2-isopropyl-pyrimidine-5-carboxamide (0.020 g, 9% yield): ¹H-NMR (300 MHz; DMSO-d₆) δ 9.84 (s, 1H), 8.88 (s, 2H), 7.40-7.32 (m, 5H), 5.99 (s, 1H), 3.91-3.63 (m, 4H), 3.16 (dt, J=13.8, 6.9 Hz, 1H), 2.46-2.36 (m, 2H), 1.27-1.23 (m, 6H); MS (ES+) m/z 440.2 (M+1).

### Example 158

### Synthesis of N-[2-(3,3-difluoropyrrolidin-1-yl)-4-(2-fluorophenyl)-3-pyridyl]-2-methyl-pyrimidine-5-carboxamide

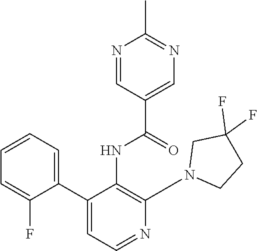 [SMILES: Cc1ncc(C(=O)Nc2c(-c3ccccc3F)ccnc2N2CCC(F)(F)C2)cn1]

To a solution of 2-chloro-N-[2-(3,3-difluoropyrrolidin-1-yl)-4-(2-fluorophenyl)-3-pyridyl]pyrimidine-5-carboxamide (0.60 g, 0.14 mmol) was added 1,4-dioxane (2.2 mL) and water (0.23 mL). The solution was sparged with nitrogen before methylboronic acid (0.055 g, 0.92 mmol), potassium carbonate (0.13 g, 0.94 mmol), and [1,1′-bis(diphenylphosphino)ferrocene]dichloropalladium(II) dichloromethane complex (0.020 g, 0.023 mmol) was added. The flask was sealed and heated to 90° C. for 4 h. The reaction mixture was diluted with ethyl acetate (150 mL), washed with saturated ammonium chloride solution (2×50 mL), dried over anhydrous magnesium sulfate, filtered and concentrated in vacuo. Purification of the residue by column chromatography, eluting with 10-50% ethyl acetate in heptane, provided the title compound as a colorless solid (0.007 g, 7% yield): ¹H-NMR (300 MHz; DMSO-d₆) δ 10.20-10.18 (m, 1H), 8.86-8.84 (m, 2H), 8.22 (dd, J=4.9, 2.2 Hz, 1H), 7.40-7.15 (m, 4H), 6.83-6.80 (m, 1H), 3.96-3.82 (m, 2H), 3.80-3.70 (m, 2H), 2.65 (s, 3H), 2.48-2.36 (m, 2H); MS (ES+) m/z 414.2 (M+1).

### Example 159

### Synthesis of N-[4-(2-fluorophenyl)-6-methyl-2-(3-oxa-8-azabicyclo[3.2.1]octan-8-yl)-3-pyridyl]-2-isopropyl-pyrimidine-5-carboxamide

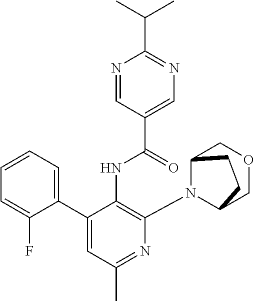 [SMILES: Cc1cc(-c2ccccc2F)c(NC(=O)c2cnc(C(C)C)nc2)c(N2[C@@H]3CC[C@H]2COC3)n1]

### Step 1. Preparation of 2-chloro-4-(2-fluorophenyl)-6-methyl-3-nitro-pyridine

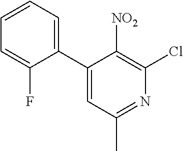 [SMILES: Cc1cc(-c2ccccc2F)c([N+](=O)[O-])c(Cl)n1]

A mixture of 2,4-dichloro-6-methyl-3-nitropyridine (3.5 g, 17 mmol), 1,4-dioxane (33 mL), and water (11 mL) was sparged with nitrogen for 10 min. The flask was added 2-fluorophenylboronic acid (2.5 g, 18 mmol), potassium carbonate (3.5 g, 25 mmol), and [1,1′-bis(diphenylphosphino)ferrocene]dichloropalladium(II), complex with dichloromethane (1.4 g, 1.7 mmol), and sparged with nitrogen for 2 min. The reaction mixture was stirred at 70° C. for 4 h. After cooling to ambient temperature the reaction mixture was diluted with ethyl acetate (200 mL), washed with saturated ammonium chloride (2×50 mL), dried over anhydrous magnesium sulfate, filtered, and concentrated in vacuo. Purification of the residue by column chromatography, eluting with 1 to 25% ethyl acetate in heptane, afforded the title compound as a colorless solid (3.1 g, 68% yield): MS (ES+) m/z 267.2 (M+1), 269.2 (M+1).

### Step 2. Preparation of 4-(2,5-difluorophenyl)-2-(3,3-difluoropyrrolidin-1-yl)-6-methyl-3-nitropyridine

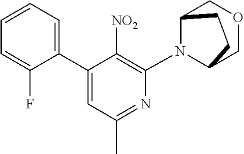 [SMILES: Cc1cc(-c2ccccc2F)c([N+](=O)[O-])c(N2[C@@H]3CC[C@H]2COC3)n1]

To a solution of 2-chloro-4-(2-fluorophenyl)-6-methyl-3-nitropyridine (0.50 g, 1.9 mmol), anhydrous potassium carbonate (0.78 g, 5.6 mmol), and 3-oxa-8-azabicyclo[3.2.1]octane hydrochloride (0.42 g, 3.8 mmol) was added N,N-dimethylformamide (6.3 mL). The reaction mixture was stirred at 50° C. for 5 h. The reaction mixture was allowed to cool to ambient temperature and diluted with ethyl acetate (200 mL), washed with saturated ammonium chloride (2×50 mL), dried over anhydrous magnesium sulfate, filtered, and concentrated in vacuo. Purification of the residue by column chromatography, eluting with 3 to 75% ethyl acetate in heptane, afforded the title compound as a yellow oil (0.58 g, 91% yield): MS (ES+) m/z 344.2 (M+1).

### Step 3. Preparation of 4-(2-fluorophenyl)-6-methyl-2-(3-oxa-8-azabicyclo[3.2.1]octan-8-yl)pyridin-3-amine

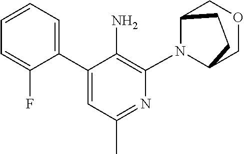 [SMILES: Cc1cc(-c2ccccc2F)c(N)c(N2[C@@H]3CC[C@H]2COC3)n1]

To a solution of 4-(2,5-difluorophenyl)-2-(3,3-difluoropyrrolidin-1-yl)-6-methyl-3-nitropyridine (0.58 g, 1.7 mmol) was added anhydrous methanol (4.2 mL), ethyl acetate (4.2 mL), and 10% palladium on carbon (0.060 g). Solid ammonium formate (2.1 g, 34 mmol) and a reflux condenser were added and the reaction mixture was heated to reflux for 25 min. After cooling to ambient temperature the reaction mixture was diluted with ethyl acetate (100 mL), filtered through diatomaceous earth (i.e., Celite®), washing with ethyl acetate (5×20 mL), and concentrated in vacuo. Purification of the residue by column chromatography, eluting with 3 to 50% ethyl acetate in heptane, afforded the title compound as a colorless solid (0.34 g, 64% yield): MS (ES+) m/z 314.2 (M+1).

### Step 4. Preparation of N-[4-(2-fluorophenyl)-6-methyl-2-(3-oxa-8-azabicyclo[3.2.1]octan-8-yl)-3-pyridyl]-2-isopropyl-pyrimidine-5-carboxamide

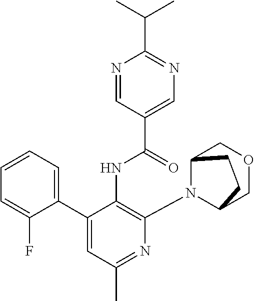 [SMILES: Cc1cc(-c2ccccc2F)c(NC(=O)c2cnc(C(C)C)nc2)c(N2[C@@H]3CC[C@H]2COC3)n1]

To a solution of 4-(2-fluorophenyl)-6-methyl-2-(3-oxa-8-azabicyclo[3.2.1]octan-8-yl)pyridin-3-amine (0.075 g, 0.31 mmol), 1-isopropylpyrimidine-5-carboxylic acid (0.052 g, 0.55 mmol) and 2-chloro-1-methylpyridinium iodide (0.21 g, 0.84 mmol) was added anhydrous tetrahydrofuran (2.4 mL). The solution was heated at 65° C. for 2 min before N-ethyl-N-isopropylpropan-2-amine (0.31 g, 2.4 mmol) was added. The reaction mixture was stirred at 65° C. for 45 min. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue column chromatography, eluting with a gradient of 20 to 100% of ethyl acetate in heptane, to provide the title compound as a colorless solid (0.045 g, 39% yield): ¹H-NMR (300 MHz; DMSO-d₆) δ 10.06 (s, 1H), 8.86 (s, 2H), 7.39-7.15 (m, 4H), 6.75 (s, 1H), 4.31 (s, 2H), 3.66 (d, J=10.3 Hz, 2H), 3.49 (dd, J=10.3, 0.8 Hz, 2H), 3.16 (quintet, J=6.9 Hz, 1H), 2.40 (s, 3H), 1.88-1.82 (m, 4H), 1.25 (t, J=5.7 Hz, 6H); MS (ES+) m/z 462.2 (M+1).

### Example 160

### Synthesis of N-[2-(3,3-difluoropyrrolidin-1-yl)-6-fluoro-4-(2-fluorophenyl)-3-pyridyl]-2-isopropyl-pyrimidine-5-carboxamide

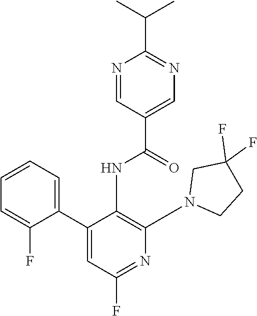 [SMILES: CC(C)c1ncc(C(=O)Nc2c(-c3ccccc3F)cc(F)nc2N2CCC(F)(F)C2)cn1]

### Step 1. Preparation of 2,6-dichloro-4-(2-fluorophenyl)-3-nitro-pyridine

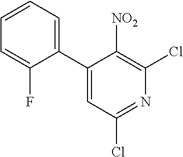 [SMILES: O=[N+]([O-])c1c(-c2ccccc2F)cc(Cl)nc1Cl]

A mixture of 2,4,6-trichloro-3-nitropyridine (1.1 g, 5.0 mmol), 1,4-dioxane (9.6 mL), and water (3.3 mL) was sparged with nitrogen for 10 min. The flask was added 2-fluorophenylboronic acid (0.70 g, 5.0 mmol), potassium carbonate (1.0 g, 7.5 mmol), and [1,1′-bis(diphenylphosphino)ferrocene]dichloropalladium(II), complex with dichloromethane (0.42 g, 0.50 mmol), and sparged with nitrogen for 2 min. The reaction mixture was stirred at 60° C. for 3 h. After cooling to ambient temperature the reaction mixture was diluted with ethyl acetate (200 mL), washed with saturated ammonium chloride (2×50 mL), dried over anhydrous magnesium sulfate, filtered, and concentrated in vacuo. Purification of the residue by column chromatography, eluting with 1-25% ethyl acetate in heptane, afforded the title compound as a slightly yellow solid (1.0 g, 70% yield): MS (ES+) m/z 287.0 (M+1), 289.0 (M+1), 291.0 (M+1).

### Step 2. Preparation of 6-chloro-2-(3,3-difluoropyrrolidin-1-yl)-4-(2-fluorophenyl)-3-nitro-pyridine

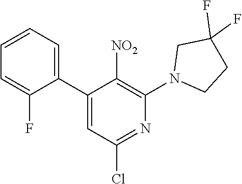 [SMILES: O=[N+]([O-])c1c(-c2ccccc2F)cc(Cl)nc1N1CCC(F)(F)C1]

To a solution of 2,6-dichloro-4-(2-fluorophenyl)-3-nitro-pyridine (1.0 g, 3.5 mmol), was added N,N-dimethylformamide (12 mL). The solution was cooled to −78° C. and anhydrous potassium carbonate (1.2 g, 8.8 mmol), and 3,3-difluoropyrrolidine hydrochloride (0.75 g, 5.3 mmol) were added. The reaction mixture was warmed to 50° C. over the course of an hour. The reaction mixture was cooled to ambient temperature and diluted with ethyl acetate (200 mL), washed with saturated ammonium chloride (2×50 mL), dried over anhydrous magnesium sulfate, filtered, and concentrated in vacuo. Purification of the residue by column chromatography, eluting with 3-50% ethyl acetate in heptane, afforded the title compound as a yellow oil that was a mixture of addition adducts (0.64 g, 51% yield): MS (ES+) m/z 358.0 (M+1), 360.0 (M+1).

### Step 3. Preparation of 2-(3,3-difluoropyrrolidin-1-yl)-6-fluoro-4-(2-fluorophenyl)-3-nitro-pyridine

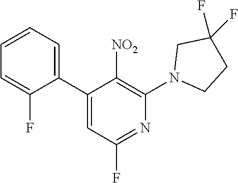 [SMILES: O=[N+]([O-])c1c(-c2ccccc2F)cc(F)nc1N1CCC(F)(F)C1]

To a solution of 6-chloro-2-(3,3-difluoropyrrolidin-1-yl)-4-(2-fluorophenyl)-3-nitro-pyridine (0.64 g, 1.8 mmol) was added anhydrous dimethylsulfoxide (18 mL). Anhydrous potassium fluoride (0.52 g, 8.9 mmol) was added, the vessel was sealed and heated to 70° C. for 18 h. The reaction mixture was added anhydrous potassium fluoride (0.52 g, 8.9 mmol) and heated to 70° C. for a further 18 h. After cooling to ambient temperature, the reaction mixture was diluted with ethyl acetate (300 mL), washed with 1 M sodium hydroxide solution (2×50 mL), brine (50 mL), dried over anhydrous magnesium sulfate, filtered, and concentrated in vacuo. Purification of the residue by column chromatography, eluting with 3 to 45% ethyl acetate in heptane, afforded the title compound as a yellow oil (7:3 mixture with the chlorinated starting material) (0.43 g, 71% yield): MS (ES+) m/z 342.0 (M+1).

### Step 4. Preparation of 2-(3,3-difluoropyrrolidin-1-yl)-6-fluoro-4-(2-fluorophenyl)pyridin-3-amine

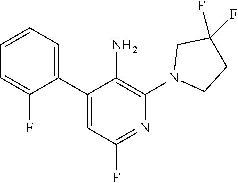 [SMILES: Nc1c(-c2ccccc2F)cc(F)nc1N1CCC(F)(F)C1]

To a solution of 2-(3,3-difluoropyrrolidin-1-yl)-6-fluoro-4-(2-fluorophenyl)-3-nitro-pyridine (0.43 g, 1.3 mmol) was added anhydrous methanol (3.2 mL) and ethyl acetate (3.2 mL). Solid ammonium formate (0.79 g, 13 mmol) and 10% palladium on carbon (0.041 g) was added. The reaction mixture was heated to reflux for 5 h. After cooling to ambient temperature, the reaction mixture was diluted with ethyl acetate (100 mL), filtered washing with ethyl acetate (3×10 mL), and concentrated in vacuo. Purification of the residue by column chromatography, eluting with 0 to 25% ethyl acetate in heptane, afforded the title compound as a colorless solid (0.21 g, 54% yield): MS (ES+) m/z312.2(M+1).

### Step 5. Preparation of N-[2-(3,3-difluoropyrrolidin-1-yl)-6-fluoro-4-(2-fluorophenyl)-3-pyridyl]-2-isopropyl-pyrimidine-5-carboxamide

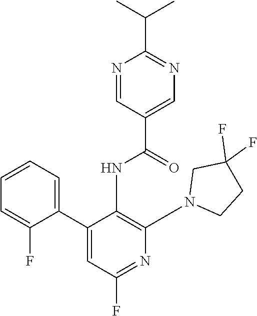 [SMILES: CC(C)c1ncc(C(=O)Nc2c(-c3ccccc3F)cc(F)nc2N2CCC(F)(F)C2)cn1]

To a solution of 2-(3,3-difluoropyrrolidin-1-yl)-6-fluoro-4-(2-fluorophenyl)pyridin-3-amine (0.21 g, 0.67 mmol), 1-isopropylpyrimidine-5-carboxylic acid (0.13 g, 0.81 mmol) and 2-chloro-1-methylpyridinium iodide (0.60 g, 2.4 mmol) was added anhydrous tetrahydrofuran (6.7 mL). The solution was heated at 65° C. for 2 min before N-ethyl-N-isopropylpropan-2-amine (0.87 g, 6.7 mmol) was added. The reaction mixture was stirred at 65° C. for 90 min. The reaction mixture was cooled to ambient temperature, diluted with ethyl acetate (200 mL), washed with saturated ammonium chloride (2×50 mL), dried over anhydrous magnesium sulfate, filtered, and concentrated in vacuo. Purification of the residue column chromatography, eluting with a gradient of 15 to 65% of ethyl acetate in heptane, to provide the title compound as a colorless solid (0.17 g, 51% yield): ¹H-NMR (300 MHz; DMSO-d₆) δ 10.11 (s, 1H), 8.85 (d, J=6.1 Hz, 2H), 7.44-7.18 (m, 4H), 6.54 (d, J=2.6 Hz, 1H), 4.04-3.94 (m, 1H), 3.92-3.75 (m, 2H), 3.73-3.62 (m, 1H), 3.16 (quintet, J=6.9 Hz, 1H), 2.48-2.38 (m, 2H), 1.28-1.24 (m, 6H); MS (ES+) m/z 460.2 (M+1).

### Example 161

### Synthesis of 4-cyano-N-[4-(2,5-difluorophenyl)-2-(3,3-difluoropyrrolidin-1-yl)-6-methyl-3-pyridylbenzamide

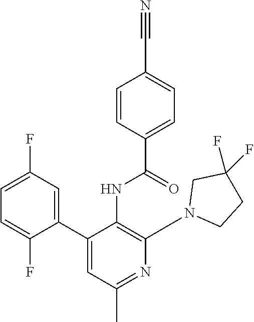 [SMILES: Cc1cc(-c2cc(F)ccc2F)c(NC(=O)c2ccc(C#N)cc2)c(N2CCC(F)(F)C2)n1]

To a solution of 4-(2,5-difluorophenyl)-2-(3,3-difluoropyrrolidin-1-yl)-6-methylpyridin-3-amine (0.087 g, 0.27 mmol), was added anhydrous tetrahydrofuran (2.7 mL), 4-cyanobenzoyl chloride (0.058 g, 0.35 mmol), N-ethyl-N-isopropylpropan-2-amine (0.35 g, 2.7 mmol). The reaction mixture was stirred at ambient temperature for 30 min. The reaction mixture was diluted with ethyl acetate (150 mL), washed with saturated ammonium chloride (2×50 mL), dried over anhydrous magnesium sulfate, filtered, and concentrated in vacuo. Purification of the residue by column chromatography, eluting with a gradient of 5 to 35% of ethyl acetate in heptane, provided the title compound as a colorless solid (0.083 g, 66% yield): ¹H-NMR (300 MHz; DMSO-d₆) δ 10.07 (s, 1H), 7.97-7.94 (m, 2H), 7.83-7.78 (m, 2H), 7.32-7.08 (m, 3H), 6.70 (s, 1H), 3.94-3.78 (m, 2H), 3.78-3.66 (m, 2H), 2.48-2.35 (m, 5H); MS (ES+) m/z 455.2 (M+1).

### Example 162

### Synthesis of N-[2-(3,3-difluoropyrrolidin-1-yl)-5-fluoro-4-(2-fluorophenyl)-3-pyridyl]-2-isopropyl-pyrimidine-5-carboxamide

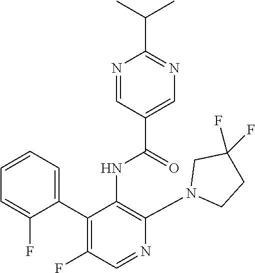 [SMILES: CC(C)c1ncc(C(=O)Nc2c(N3CCC(F)(F)C3)ncc(F)c2-c2ccccc2F)cn1]

### Step 1. Preparation of 2-bromo-5-fluoro-4-(2-fluorophenyl)-3-nitro-pyridine

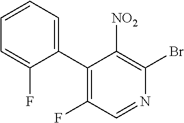 [SMILES: O=[N+]([O-])c1c(Br)ncc(F)c1-c1ccccc1F]

To 2,4-dibromo-5-fluoro-3-nitropyridine (0.37 g, 1.2 mmol) was added 1,4-dioxane (2.4 mL), and water (0.83 mL) and the mixture was sparged with nitrogen for 10 min. To the mixture was added 2-fluorophenylboronic acid (0.21 g, 1.5 mmol), potassium carbonate (0.29 g, 2.1 mmol), and [1,1′-bis(diphenylphosphino)ferrocene]dichloropalladium(II), complex with dichloromethane (0.11 g, 0.12 mmol), and sparged with nitrogen for 2 min. The vial was sealed and heated to 65° C. for 1 h. After cooling to ambient temperature the reaction mixture was diluted with ethyl acetate (200 mL), washed with saturated ammonium chloride (2×50 mL), dried over anhydrous magnesium sulfate, filtered, and concentrated in vacuo. Purification of the residue by column chromatography, eluting with 0 to 20% ethyl acetate in heptane, afforded the title compound as a colorless oil (0.15 g, 39% yield): MS (ES+) m/z 315.0 (M+1), 317.0 (M+1).

### Step 2. Preparation of 2-(3,3-difluoropyrrolidin-1-yl)-5-fluoro-4-(2-fluorophenyl)-3-nitro-pyridine

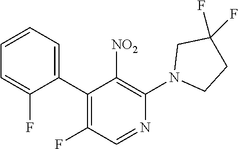 [SMILES: O=[N+]([O-])c1c(N2CCC(F)(F)C2)ncc(F)c1-c1ccccc1F]

To 2-bromo-5-fluoro-4-(2-fluorophenyl)-3-nitro-pyridine (0.15 g, 0.49 mmol) was added 1-methyl-2-pyrrolidinone (1.6 mL), anhydrous potassium carbonate (0.34 g, 2.4 mmol), and 3,3-difluoropyrrolidine hydrochloride (0.14 g, 0.98 mmol). The vial was sealed and heated to 70° C. for 10 h. The reaction mixture was cooled to ambient temperature and diluted with ethyl acetate (160 mL) and washed with saturated ammonium chloride (2×50 mL), water (50 mL), and brine (50 mL). The organic phase was dried over anhydrous magnesium sulfate, filtered, and concentrated in vacuo. Purification of the residue by column chromatography, eluting with 2 to 25% ethyl acetate in heptane, afforded the title compound as a yellow oil that was a mixture of addition adducts (0.058 g, 35% yield): MS (ES+) m/z 342.2 (M+1).

### Step 3. Preparation of N-[2-(3,3-difluoropyrrolidin-1-yl)-5-fluoro-4-(2-fluorophenyl)-3-pyridyl]-2-isopropyl-pyrimidine-5-carboxamide

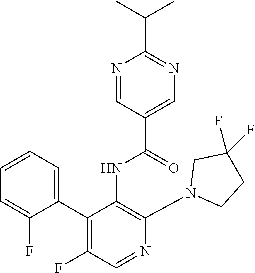 [SMILES: CC(C)c1ncc(C(=O)Nc2c(N3CCC(F)(F)C3)ncc(F)c2-c2ccccc2F)cn1]

To a solution of 2-(3,3-difluoropyrrolidin-1-yl)-5-fluoro-4-(2-fluorophenyl)-3-nitro-pyridine (0.058 g, 0.17 mmol) was added anhydrous methanol (0.56 mL), ethyl acetate (0.56 mL), and 10% palladium on carbon (0.006 g). Ammonium formate (0.21 g, 3.4 mmol) was added and the mixture was heated to reflux for 30 min. After cooling to ambient temperature the reaction mixture was diluted with ethyl acetate (50 mL), filtered through diatomaceous earth (i.e., Celite®), washed with ethyl acetate (3×10 mL) and concentrated in vacuo. The residue was dissolved in a mixture of anhydrous tetrahydrofuran (1.7 mL), 1-isopropylpyrimidine-5-carboxylic acid (0.034 g, 0.20 mmol) and 2-chloro-1-methylpyridinium iodide (0.15 g, 0.59 mmol). The solution was heated at 65° C. for 2 min before N-ethyl-N-isopropylpropan-2-amine (0.22 g, 0.30 mmol) was added. The reaction mixture was stirred at 65° C. for 6 h. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue column chromatography, eluting with a gradient of 10 to 75% of ethyl acetate in heptane, to provide the title compound as a colorless solid (0.020 g, 24% yield): ¹H-NMR (300 MHz; DMSO-d₆) δ 10.33 (s, 1H), 8.87 (d, J=4.9 Hz, 2H), 8.35 (s, 1H), 7.48-7.40 (m, 1H), 7.36-7.28 (m, 2H), 7.22 (td, J=7.4, 1.1 Hz, 1H), 3.92-3.79 (m, 2H), 3.77-3.66 (m, 2H), 3.16 (dt, J=13.8, 6.9 Hz, 1H), 2.49-2.37 (m, 2H), 1.28-1.24 (m, 6H); MS (ES+) m/z 460.2 (M+1).

### Example 163

### Synthesis of N-[6-fluoro-4-(2-fluorophenyl)-2-pyrrolidin-1-yl-3-pyridyl]-2-isopropyl-pyrimidine-5-carboxamide

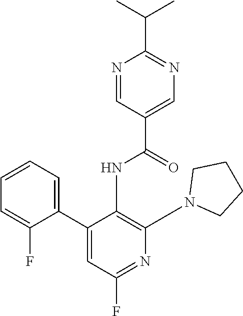 [SMILES: CC(C)c1ncc(C(=O)Nc2c(-c3ccccc3F)cc(F)nc2N2CCCC2)cn1]

### Step 1. Preparation of 2,6-difluoro-4-(2-fluorophenyl)-3-nitro-pyridine

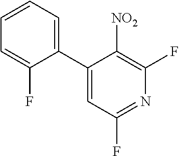 [SMILES: O=[N+]([O-])c1c(-c2ccccc2F)cc(F)nc1F]

To a solution of 2,6-dichloro-4-(2-fluorophenyl)-3-nitro-pyridine (1.6 g, 5.6 mmol) was added anhydrous dimethylsulfoxide (28 mL). Anhydrous potassium fluoride (8.1 g, 139 mmol) was added, the vessel was sealed, and heated to 90° C. for 20 h. The reaction mixture was added anhydrous potassium fluoride (0.52 g, 8.9 mmol) and heated to 70° C. for a further 18 h. After cooling to ambient temperature, the reaction mixture was diluted with ethyl acetate (200 mL), washed with saturated sodium bicarbonate (2×50 mL) and brine (50 mL). The organic phase was dried over anhydrous magnesium sulfate, filtered, and concentrated in vacuo. Purification of the residue by column chromatography, eluting with 1 to 15% ethyl acetate in heptane, afforded the title compound as a yellow oil (0.65 g, 46% yield): MS (ES+) m/z 256.2 (M+1).

### Step 2. Preparation of 6-fluoro-4-(2-fluorophenyl)-3-nitro-2-pyrrolidin-1-yl-pyridine

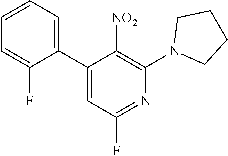 [SMILES: O=[N+]([O-])c1c(-c2ccccc2F)cc(F)nc1N1CCCC1]

To a solution of 2,6-difluoro-4-(2-fluorophenyl)-3-nitro-pyridine (0.15 g, 0.59 mmol) was added anhydrous 1-methyl-2-pyrrolidinone (5.9 mL). The solution was cooled to 0° C. before N-ethyl-N-isopropylpropan-2-amine (0.23 g, 1.8 mmol) and pyrrolidine (0.040 g, 0.56 mmol) were added. The reaction mixture was allowed to warm to ambient temperature and stir for 10 min. The reaction mixture was diluted with ethyl acetate (150 mL), washed with saturated ammonium chloride (2×50 mL), dried over anhydrous magnesium sulfate, filtered, and concentrated in vacuo. Purification of the residue by column chromatography, eluting with 2 to 20% ethyl acetate in heptane, afforded the title compound as a yellow oil (0.11 g,60% yield): MS (ES+) m/z 306.0 (M+1).

### Step 3. Preparation of 6-fluoro-4-(2-fluorophenyl)-2-pyrrolidin-1-yl-pyridin-3-amine

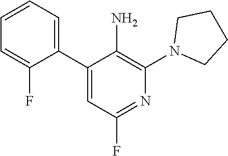 [SMILES: Nc1c(-c2ccccc2F)cc(F)nc1N1CCCC1]

To a solution of 6-fluoro-4-(2-fluorophenyl)-3-nitro-2-pyrrolidin-1-yl-pyridine (0.11 g, 0.36 mmol) was added anhydrous methanol (1.2 mL), ethyl acetate (1.2 mL), and 10% palladium on carbon (0.011 g). Ammonium formate (0.45 g, 7.1 mmol) was added and the reaction mixture was heated to reflux 18 h. After cooling to ambient temperature, the reaction mixture was diluted with ethyl acetate (100 mL), filtered through diatomaceous earth (i.e., Celite®), washed with ethyl acetate (3×10 mL) and concentrated in vacuo. Purification by column chromatography, eluting with a gradient of 2 to 30% ethyl acetate in heptane, afforded the title compound as a colorless oil (0.075 g, 76% yield): MS (ES+) m/z 276.2 (M+1).

### Step 4. Preparation of N-[6-fluoro-4-(2-fluorophenyl)-2-pyrrolidin-1-yl-3-pyridyl]-2-isopropyl-pyrimidine-5-carboxamide

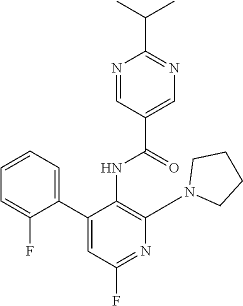 [SMILES: CC(C)c1ncc(C(=O)Nc2c(-c3ccccc3F)cc(F)nc2N2CCCC2)cn1]

To 6-fluoro-4-(2-fluorophenyl)-2-pyrrolidin-1-yl-pyridin-3-amine (0.065 g, 0.24 mmol), 1-isopropylpyrimidine-5-carboxylic acid (0.047 g, 0.28 mmol) and 2-chloro-1-methylpyridinium iodide (0.15 g, 0.59 mmol) was added anhydrous tetrahydrofuran (12 mL). The solution was heated at 65° C. for 2 min before N-ethyl-N-isopropylpropan-2-amine (0.30 g, 2.4 mmol) was added. The reaction mixture was stirred at 65° C. for 60 min. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue by column chromatography, eluting with a gradient of 10 to 40% of ethyl acetate in heptane, provided the title compound as a colorless solid (0.075 g, 74% yield): ¹H-NMR (300 MHz; DMSO-d₆) δ 10.04 (s, 1H), 8.84 (d, J=2.9 Hz, 2H), 7.39 (dddd, J=8.3, 7.2, 5.4, 1.9 Hz, 1H), 7.33-7.23 (m, 2H), 7.23-7.16 (m, 1H), 6.33 (d, J=2.7 Hz, 1H), 3.64-3.56 (m, 2H), 3.43-3.35 (m, 2H), 3.15 (quintet, J=6.9 Hz, 1H), 1.82 (t, J=6.4 Hz, 4H), 1.28-1.24 (m, 6H); MS (ES+) m/z 424.2 (M+1).

### Example 164

### Synthesis of N-[4-(2,5-difluorophenyl)-2-(3,3-difluoropyrrolidin-1-yl)-6-methyl-3-pyridyl]-2-methoxy-pyrimidine-5-carboxamide

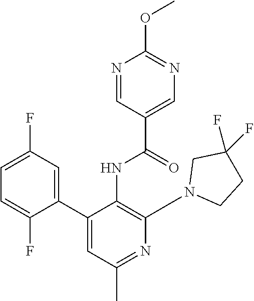 [SMILES: COc1ncc(C(=O)Nc2c(-c3cc(F)ccc3F)cc(C)nc2N2CCC(F)(F)C2)cn1]

To a solution of 4-(2,5-difluorophenyl)-2-(3,3-difluoropyrrolidin-1-yl)-6-methylpyridin-3-amine (0.060 g, 0.18 mmol), was added anhydrous tetrahydrofuran (3.7 mL), and 2-methoxypyrimidine-5-carboxylic acid (0.034 g, 0.22 mmol). The reaction mixture was heated to 65° C. for 1 min prior to the addition of N-ethyl-N-isopropylpropan-2-amine (0.24 g, 1.8 mmol). The reaction mixture was stirred at 65° C. for 2 h. The reaction mixture was cooled to ambient temperature, diluted with ethyl acetate (150 mL), washed with saturated ammonium chloride (2×50 mL), dried over anhydrous magnesium sulfate, filtered, and concentrated in vacuo. Purification of the residue by column chromatography, eluting with a gradient of 5 to 50% of ethyl acetate in heptane, to provide the title compound as a colorless solid (0.047 g, 53% yield): ¹H-NMR (300 MHz; DMSO-d₆) δ 9.99 (s, 1H), 8.85 (s, 2H), 7.30 (td, J=9.1, 4.6 Hz, 1H), 7.24-7.16 (m, 1H), 7.12 (ddd, J=8.8, 5.6, 3.2 Hz, 1H), 6.70 (s, 1H), 3.95 (s, 3H), 3.95-3.81 (m, 2H), 3.75-3.69 (m, 2H), 2.47-2.35 (m, 5H); MS (ES+) m/z 462.2 (M+1).

### Example 165

### Synthesis of N-[2-(3,3-difluoropyrrolidin-1-yl)-4-(2-fluorophenyl)-3-pyridyl]-6-(1-hydroxyethyl)pyridine-3-carboxamide

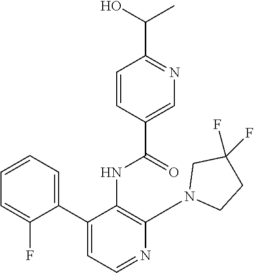 [SMILES: CC(O)c1ccc(C(=O)Nc2c(-c3ccccc3F)ccnc2N2CCC(F)(F)C2)cn1]

### Step 1. Preparation of N-[2-(3,3-difluoropyrrolidin-1-yl)-4-(2-fluorophenyl)-3-pyridyl]-6-(1,3-dioxolan-2-yl)pyridine-3-carboxamide

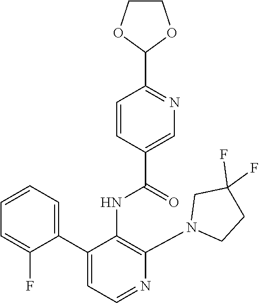 [SMILES: O=C(Nc1c(-c2ccccc2F)ccnc1N1CCC(F)(F)C1)c1ccc(C2OCCO2)nc1]

To a solution of 2-(3,3-difluoropyrrolidin-1-yl)-4-(2-fluorophenyl)pyridin-3-amine (0.56 g, 1.9 mmol), 6-(1,3-dioxalan-2-yl)pyridine-3-carboxylic acid (0.41 g, 2.1 mmol) and 2-chloro-1-methylpyridinium iodide (1.2 g, 4.8 mmol) was added anhydrous tetrahydrofuran (38 mL). The solution was heated to reflux for 2 min before N-ethyl-N-isopropylpropan-2-amine (2.5 g, 19 mmol) was added. The reaction mixture was heated to reflux for 4 h. The reaction mixture was cooled to ambient temperature, diluted with ethyl acetate (200 mL), washed with saturated ammonium chloride solution (2×50 mL), dried over anhydrous magnesium sulfate, filtered and concentrated in vacuo. Purification by column chromatography, eluting with a gradient of 15 to 100% ethyl acetate in heptane, provided the title compound as a colorless solid (0.66 g, 73% yield): MS (ES+) m/z 471.2 (M+1).

### Step 2. Preparation of N-[2-(3,3-difluoropyrrolidin-1-yl)-4-(2-fluorophenyl)-3-pyridyl]-6-formyl-pyridine-3-carboxamide

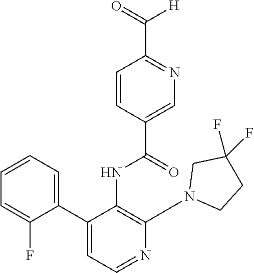 [SMILES: O=Cc1ccc(C(=O)Nc2c(-c3ccccc3F)ccnc2N2CCC(F)(F)C2)cn1]

To a solution of N-[2-(3,3-difluoropyrrolidin-1-yl)-4-(2-fluorophenyl)-3-pyridyl]-6-(1,3-dioxolan-2-yl)pyridine-3-carboxamide (0.56 g, 1.9 mmol) was added methanol (20 mL) and concentrated hydrochloric acid (2 mL). The reaction mixture was heated to reflux for 3 h. Concentrated hydrochloric acid (2 mL) was added every 3 h until the starting material was consumed. The reaction mixture was cooled to ambient temperature and diluted with ethyl acetate (300 mL) and water (50 mL). The pH was adjusted to >8 with a 50% sodium hydroxide solution, separated then dried over anhydrous magnesium sulfate, filtered and concentrated in vacuo. Purification by column chromatography, eluting with a gradient of 20 to 100% ethyl acetate in heptane, provided the title compound as a yellow solid (0.45 g, 75% yield): MS (ES+) m/z 427.2 (M+1).

### Step 3. Preparation of N-[2-(3,3-difluoropyrrolidin-1-yl)-4-(2-fluorophenyl)-3-pyridyl]-6-(1-hydroxyethyl)pyridine-3-carboxamide

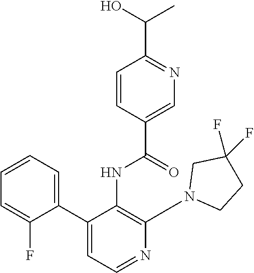 [SMILES: CC(O)c1ccc(C(=O)Nc2c(-c3ccccc3F)ccnc2N2CCC(F)(F)C2)cn1]

To N-[2-(3,3-difluoropyrrolidin-1-yl)-4-(2-fluorophenyl)-3-pyridyl]-6-formyl-pyridine-3-carboxamide (0.056 g, 0.13 mmol) was added anhydrous tetrahydrofuran (0.70 mL). To the mixture was added methylmagnesium bromide in tetrahydrofuran (0.090 mL, 3 M) at ambient temperature and the mixture was stirred for 30 min. Anhydrous methanol (0.50 mL) and concentrated acetic acid (0.10 mL) were added and the reaction mixture was concentrated in vacuo. Purification by column chromatography, eluting with a gradient of 15 to 100% ethyl acetate in heptane, provided the title compound as a colorless solid (0.042 g, 70% yield): ¹H-NMR (300 MHz; DMSO-d₆) δ 10.02 (s, 1H), 8.69 (dd, J=2.3, 0.8 Hz, 1H), 8.20 (d, J=5.0 Hz, 1H), 8.00 (dd, J=8.2, 2.3 Hz, 1H), 7.57-7.54 (m, 1H), 7.39-7.14 (m, 4H), 6.80 (dd, J=5.0, 0.8 Hz, 1H), 5.47 (d, J=4.6 Hz, 1H), 4.77-4.69 (m, 1H), 3.97-3.81 (m, 2H), 3.80-3.69 (m, 2H), 2.48-2.36 (m, 2H), 1.35 (d, J=6.6 Hz, 3H); MS (ES+) m/z 443.2 (M+1).

### Example 166

### Synthesis of N-[2-(3,3-difluoropyrrolidin-1-yl)-4-(2-fluorophenyl)-3-pyridyl]-6-(2,2,2-trifluoro-1-hydroxy-ethyl)pyridine-3-carboxamide

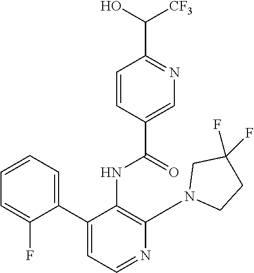 [SMILES: O=C(Nc1c(-c2ccccc2F)ccnc1N1CCC(F)(F)C1)c1ccc(C(O)C(F)(F)F)nc1]

To N-[2-(3,3-difluoropyrrolidin-1-yl)-4-(2-fluorophenyl)-3-pyridyl]-6-formyl-pyridine-3-carboxamide (0.052 g, 0.12 mmol) was added anhydrous tetrahydrofuran (0.20 mL) and trimethyl(trifluoromethyl)silane (0.021 g, 0.15 mmol). A 1 M solution of tetrabutylammonium fluoride in tetrahydrofuran (0.006 mL, 0.006 mmol) was added and the reaction mixture was stirred for 2 h. Further trimethyl(trifluoromethyl)silane (0.042 g, 0.30 mmol) and 1 M solution of tetrabutylammonium fluoride in tetrahydrofuran (0.030 mL, 0.030 mmol) was added and the reaction mixture was stirred for 18 h. The reaction mixture was diluted with ethyl acetate (200 mL), washed with saturated sodium bicarbonate (2×50 mL), dried over anhydrous magnesium sulfate, filtered, and concentrated in vacuo. Purification by column chromatography, eluting with a gradient of 0 to 60% ethyl acetate in heptane, provided the title compound as a colorless solid (0.020 g, 33% yield): ¹H-NMR (300 MHz; DMSO-d₆) δ 10.20-10.14 (m, 1H), 8.77-8.72 (m, 1H), 8.24-8.19 (m, 1H), 8.11-8.06 (m, 1H), 7.73-7.67 (m, 1H), 7.41-7.23 (m, 3H), 7.23-7.13 (m, 2H), 6.84-6.80 (m, 1H), 5.24-5.14 (m, 1H), 4.02-3.82 (m, 2H), 3.82-3.63 (m, 2H), 2.48-2.36 (m, 2H); MS (ES+) m/z 497.2 (M+1).

### Example 167

### Synthesis of N-5-[2-(3,3-difluoropyrrolidin-1-yl)-4-(2-fluorophenyl)-3-pyridyl]-N2-methyl-pyridine-2,5-dicarboxamide

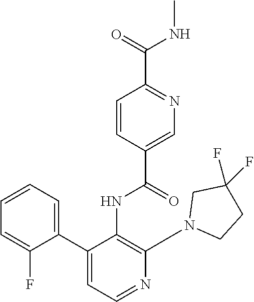 [SMILES: CNC(=O)c1ccc(C(=O)Nc2c(-c3ccccc3F)ccnc2N2CCC(F)(F)C2)cn1]

### Step 1. Preparation of 5-[[2-(3,3-difluoropyrrolidin-1-yl)-4-(2-fluorophenyl)-3-pyridyl]carbamoyl]pyridine-2-carboxylic acid

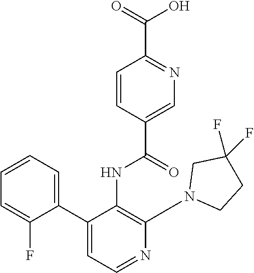 [SMILES: O=C(Nc1c(-c2ccccc2F)ccnc1N1CCC(F)(F)C1)c1ccc(C(=O)O)nc1]

To a solution of N-[2-(3,3-difluoropyrrolidin-1-yl)-4-(2-fluorophenyl)-3-pyridyl]-6-formyl-pyridine-3-carboxamide (0.14 g, 0.32 mmol) was added dichloromethane (1.1 mL), 2-methyl-2-butene (1.0 mL), and tert-butanol (4.0 mL). The solution was cooled to 0° C. before a mixture of sodium chlorite (0.091 g, 0.81 mmol), sodium dihydrogenphosphate (0.14 g, 1.1 mmol), and water (1.8 mL) was added dropwise. The solution was stirred at 0° C. for 4 h. The reaction mixture was diluted with ethyl acetate (200 mL) and washed with 1 M hydrochloric acid (10 mL). The aqueous layer was extracted with ethyl acetate (3×50 mL) and the combined organic fractions were dried over anhydrous magnesium sulfate, filtered, and concentrated in vacuo. The light yellow oil was used in the next step without further purification (0.14 g, 99% yield): MS (ES+) m/z 443.2 (M+1).

### Step 2. Preparation of N5-[2-(3,3-difluoropyrrolidin-1-yl)-4-(2-fluorophenyl)-3-pyridyl]-N2-methyl-pyridine-2,5-dicarboxamide

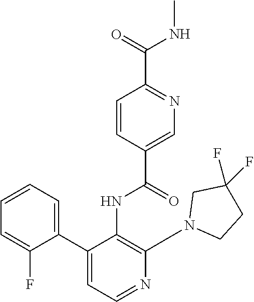 [SMILES: CNC(=O)c1ccc(C(=O)Nc2c(-c3ccccc3F)ccnc2N2CCC(F)(F)C2)cn1]

To 5-[[2-(3,3-difluoropyrrolidin-1-yl)-4-(2-fluorophenyl)-3-pyridyl]carbamoyl]pyridine-2-carboxylic acid (0.14 g, 0.32 mmol) and HATU (0.19 g, 5 0.48 mmol) was added anhydrous N,N-dimethylformamide (0.81 mL). N-Ethyl-N-isopropylpropan-2-amine (0.21 g, 1.6 mmol) was added and the reaction mixture was stirred at ambient temperature for 10 min, followed by the addition of methylamine hydrochloride (0.11 g, 1.6 mmol). The reaction mixture was stirred at ambient temperature for 30 min before being diluted with ethyl acetate (200 mL), washed with saturated ammonium chloride (2×50 mL), dried over anhydrous magnesium sulfate, filtered, and concentrated in vacuo. Purification by column chromatography, eluting with a gradient of 25 to 100% ethyl acetate in heptane, provided the title compound as a colorless solid (0.020 g, 33% yield): ¹H-NMR (300 MHz; DMSO-d₆) δ 10.23 (s, 1H), 8.93-8.84 (m, 1H), 8.77-8.73 (m, 1H), 8.24-8.20 (m, 1H), 8.20-8.15 (m, 1H), 8.08-8.04 (m, 1H), 7.41-7.28 (m, 2H), 7.27-7.14 (m, 2H), 6.83-6.80 (m, 1H), 4.02-3.82 (m, 2H), 3.82-3.69 (m, 2H), 2.85-2.78 (m, 3H), 2.49-2.36 (m, 2H); MS (ES+) m/z 456.2 (M+1).

### Example 168

### Synthesis of N-[6-cyano-2-(3,3-difluoropyrrolidin-1-yl)-4-(2-fluorophenyl)-3-pyridyl]-2-isopropyl-pyrimidine-5-carboxamide

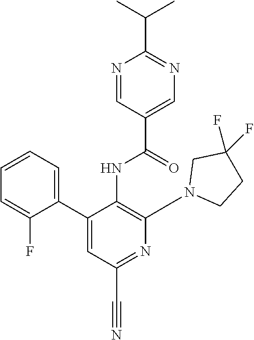 [SMILES: CC(C)c1ncc(C(=O)Nc2c(-c3ccccc3F)cc(C#N)nc2N2CCC(F)(F)C2)cn1]

### Step 1. Preparation of 6-(3,3-difluoropyrrolidin-1-yl)-4-(2-fluorophenyl)-5-nitro-pyridine-2-carbonitrile

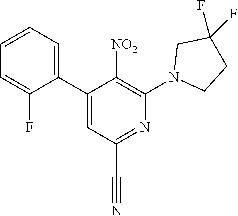 [SMILES: N#Cc1cc(-c2ccccc2F)c([N+](=O)[O-])c(N2CCC(F)(F)C2)n1]

To a solution of 2-(3,3-difluoropyrrolidin-1-yl)-6-fluoro-4-(2-fluorophenyl)-3-nitro-pyridine (0.060 g, 0.18 mmol) was added anhydrous 1-methyl-2-pyrrolidinone (1.8 mL) and sodium cyanide (0.043 g, 0.88 mmol). The reaction mixture was stirred at ambient temperature for 1 h before being diluted with ethyl acetate (100 mL), washed with water (3×50 mL), brine (50 mL), dried over anhydrous magnesium sulfate, filtered, and concentrated in vacuo. Purification of the residue by column chromatography, eluting with 0 to 50% ethyl acetate in heptane, afforded the title compound as a colorless solid (0.061 g, 99% yield): MS (ES+) m/z 349.0 (M+1).

### Step 2. Preparation of 5-amino-6-(3,3-difluoropyrrolidin-1-yl)-4-(2-fluorophenyl)pyridine-2-carbonitrile

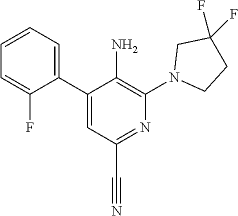 [SMILES: N#Cc1cc(-c2ccccc2F)c(N)c(N2CCC(F)(F)C2)n1]

To a solution of 2-(3,3-difluoropyrrolidin-1-yl)-6-fluoro-4-(2-fluorophenyl)-3-nitro-pyridine (0.040 g, 0.11 mmol) was added ethyl acetate (0.50 mL) and tin dichloride dehydrate (0.078 g, 0.34 mmol). The flask was sealed and heated to 50° C. for 90 min before ethyl aceate (2.0 mL) and tin dichloride dihydrate (0.050 g, 0.23 mmol) was added. The reaction mixture was stirred at 50° C. for 18 h. After cooling to ambient temperature, the reaction mixture was diluted with ethyl acetate (200 mL), washed with saturated ammonium chloride (3×50 mL), dried over anhydrous magnesium sulfate, filtered, and concentrated in vacuo. Purification of the residue by column chromatography, eluting with 0 to 40% ethyl acetate in heptane, afforded the title compound as a colorless oil (0.020 g, 55% yield): MS (ES+) m/z 319.2 (M+1).

### Step 3. Preparation of N-[6-cyano-2-(3,3-difluoropyrrolidin-1-yl)-4-(2-fluorophenyl)-3-pyridyl]-2-isopropyl-pyrimidine-5-carboxamide

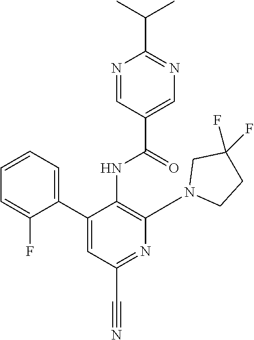 [SMILES: CC(C)c1ncc(C(=O)Nc2c(-c3ccccc3F)cc(C#N)nc2N2CCC(F)(F)C2)cn1]

To a solution of 5-amino-6-(3,3-difluoropyrrolidin-1-yl)-4-(2-fluorophenyl)pyridine-2-carbonitrile (0.020 g, 0.063 mmol), 1-isopropylpyrimidine-5-carboxylic acid (0.012 g, 0.069 mmol) and 2-chloro-1-methylpyridinium iodide (0.040 g, 0.16 mmol) was added anhydrous tetrahydrofuran (1.3 mL). The solution was heated at 65° C. for 2 min before N-ethyl-N-isopropylpropan-2-amine (0.081 g, 0.63 mmol) was added. The reaction mixture was stirred at 65° C. for 18 h. The reaction mixture was cooled to ambient temperature and diluted with methanol (3.0 mL) and 5 M sodium hydroxide (2.0 mL). The mixture was stirred for 10 min before being diluted with ethyl acetate (125 mL), washed with saturated ammonium chloride (2×50 mL), dried over anhydrous magnesium sulfate, filtered, and concentrated in vacuo. Purification of the residue column chromatography, eluting with a gradient of 15 to 100% of ethyl acetate in heptane, to provide the title compound as a colorless solid (0.013 g, 42% yield): ¹H-NMR (300 MHz; DMSO-d₆) δ 10.43 (s, 1H), 8.87 (s, 2H), 7.50 (d, J=0.5 Hz, 1H), 7.46-7.39 (m, 1H), 7.36-7.29 (m, 2H), 7.27-7.20 (m, 1H), 3.99-3.88 (m, 2H), 3.85-3.74 (m, 2H), 3.17 (dt, J=13.8, 6.9 Hz, 1H), 2.52-2.39 (m, 2H), 1.28-1.25 (m, 6H); MS (ES+) m/z467.2(M+1).

### Example 169

### Synthesis of N-[2-(3,3-difluoropyrrolidin-1-yl)-4-phenyl-6-(trifluoromethyl)-3-pyridyl]-2-isopropyl-pyrimidine-5-carboxamide

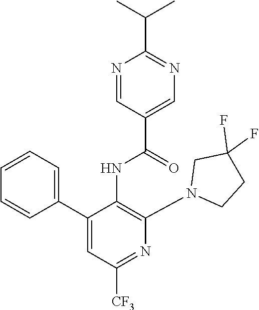 [SMILES: CC(C)c1ncc(C(=O)Nc2c(-c3ccccc3)cc(C(F)(F)F)nc2N2CCC(F)(F)C2)cn1]

### Step 1. Preparation of 2-chloro-4-phenyl-6-(trifluoromethyl)pyridin-3-amine

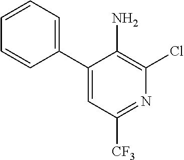 [SMILES: Nc1c(-c2ccccc2)cc(C(F)(F)F)nc1Cl]

A mixture of 4-bromo-2-chloro-6-(trifluoromethyl)pyridin-3-amine (1.0 g, 3.7 mmol), 1,4-dioxane (7.4 mL), and water (3.7 mL) was sparged with nitrogen for 10 min. The flask was added phenylboronic acid (0.68 g, 5.6 mmol), potassium carbonate (1.2 g, 8.9 mmol), and [1,1′-bis(diphenylphosphino)ferrocene]dichloropalladium(II), complex with dichloromethane (0.31 g, 0.37 mmol), and sparged with nitrogen for 2 min. The reaction mixture was stirred at 90° C. for 2 h. After cooling to ambient temperature the reaction mixture was diluted with ethyl acetate (150 mL), washed with saturated ammonium chloride (2×50 mL), dried over anhydrous magnesium sulfate, filtered, and concentrated in vacuo. Purification of the residue by column chromatography, eluting with 0 to 25% ethyl acetate in heptane, afforded the title compound as a colorless solid (0.96 g, 95% yield): MS (ES+) m/z 287.0 (M+1), 289.0 (M+1).

### Step 2. Preparation of 2-(3,3-difluoropyrrolidin-1-yl)-4-phenyl-6-(trifluoromethyl)pyridin-3-amine

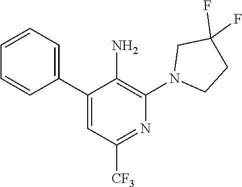 [SMILES: Nc1c(-c2ccccc2)cc(C(F)(F)F)nc1N1CCC(F)(F)C1]

To 2-chloro-4-phenyl-6-(trifluoromethyl)pyridin-3-amine (0.86 g, 2.8 mmol), was added 1-methyl-2-pyrrolidinone (9.3 mL), 3,3-difluoropyrrolidine hydrochloride (3.2 g, 22 mmol), and N-ethyl-N-isopropylpropan-2-amine (7.2 g, 56 mmol) before being sealed. The solution was heated in a microwave to 200° C. for 90 min. The reaction mixture was cooled to ambient temperature and diluted with ethyl acetate (250 mL), washed with saturated ammonium chloride (2×50 mL), dried over anhydrous magnesium sulfate, filtered, and concentrated in vacuo. Purification of the residue by column chromatography, eluting with 0 to 60% ethyl acetate in heptane, afforded the title compound as a brown oil (0.92 g, 96% yield): ¹H-NMR (300 MHz; DMSO-d₆) δ 7.56-7.43 (m, 5H), 7.24 (s, 1H), 4.12 (s, 2H), 3.77 (t, J=13.0 Hz, 2H), 3.63 (t, J=7.2 Hz, 2H), 2.48 (tt, J=14.3, 7.2 Hz, 2H); MS (ES+) m/z 344.2 (M+1).

### Step 3. Preparation of N-[2-(3,3-difluoropyrrolidin-1-yl)-4-phenyl-6-(trifluoromethyl)-3-pyridyl]-2-isopropyl-pyrimidine-5-carboxamide

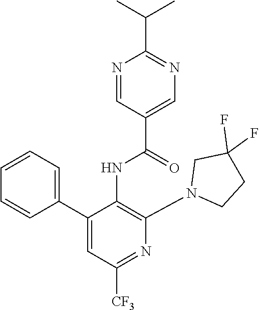 [SMILES: CC(C)c1ncc(C(=O)Nc2c(-c3ccccc3)cc(C(F)(F)F)nc2N2CCC(F)(F)C2)cn1]

To a solution of 2-(3,3-difluoropyrrolidin-1-yl)-4-phenyl-6-(trifluoromethyl)pyridin-3-amine (0.23 g, 0.66 mmol), 1-isopropylpyrimidine-5-carboxylic acid (0.12 g, 0.72 mmol) and 2-chloro-1-methylpyridinium iodide (0.42 g, 1.6 mmol) was added anhydrous tetrahydrofuran (13 mL). The solution was heated at 65° C. for 2 min before N-ethyl-N-isopropylpropan-2-amine (0.85 g, 6.6 mmol) was added. The reaction mixture was stirred at 65° C. for 9 h. After cooling to ambient temperature, methanol (5 mL) and 5 M sodium hydroxide (4 mL) was added and stirred for 30 min. The reaction mixture was diluted with ethyl acetate (150 mL), washed with saturated ammonium chloride (2×50 mL), dried over anhydrous magnesium sulfate, filtered, and concentrated in vacuo.

Purification of the residue by column chromatography, eluting with a gradient of 10 to 100% of ethyl acetate in heptane, followed by preparative HPLC, eluting with 40 to 85% of acetonitrile in water containing 0.5% formic acid, provided the title compound as a colorless solid (0.072 g, 22% yield): ¹H-NMR (300 MHz; DMSO-d₆) δ 10.39 (d, J=6.6 Hz, 1H), 8.93 (d, J=4.8 Hz, 2H), 7.47-7.34 (m, 5H), 7.19 (s, 1H), 4.06-3.69 (m, 4H), 3.22-3.13 (m, 1H), 2.54-2.40 (m, 2H), 1.27 (d, J=6.9 Hz, 6H); MS (ES+) m/z 492.2 (M+1).

### Example 170

### Synthesis of 2-(cyclopropoxy)-N-[2-(3,3-difluoropyrrolidin-1-yl)-4-(2-fluorophenyl)-3-pyridyl]pyrimidine-5-carboxamide

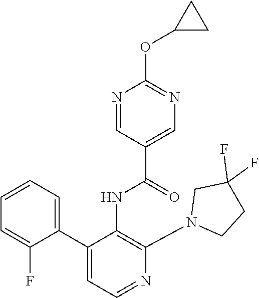 [SMILES: O=C(Nc1c(-c2ccccc2F)ccnc1N1CCC(F)(F)C1)c1cnc(OC2CC2)nc1]

To 2-chloro-N-[2-(3,3-difluoropyrrolidin-1-yl)-4-(2-fluorophenyl)-3-pyridyl]pyrimidine-5-carboxamide (0.10 g, 0.23 mmol) was added anhydrous 1-methyl-2-pyrrolidinone (0.50 mL) and cyclopropanol (0.067 g, 1.2 mmol). Sodium hydride, 60% dispersion in mineral oil, (0.046 g, 1.2 mol), was added, the vial was sealed, and heated to 50° C. for 1 h. The reaction mixture was diluted with ethyl acetate (200 mL), washed with saturated ammonium chloride solution (2×50 mL), dried over anhydrous magnesium sulfate, filtered and concentrated in vacuo Purification of the residue by preparative HPLC, eluting with a gradient of 15 to 85% acetonitrile in water containing 0.5% formic acid, provided the title compound as a colorless solid (0.038 g, 35% yield): ¹H-NMR (400 MHz; DMSO-d₆) δ 10.07 (s, 1H), 8.81 (d, J=6.1 Hz, 2H), 8.21 (d, J=5.0 Hz, 1H), 7.37 (dddd, J=8.5, 7.1, 5.4, 1.6 Hz, 1H), 7.30 (td, J=7.5, 1.5 Hz, 1H), 7.28-7.22 (m, 1H), 7.20-7.16 (m, 1H), 6.81 (d, J=4.9 Hz, 1H), 4.37-4.33 (m, 1H), 3.96-3.83 (m, 2H), 3.83-3.71 (m, 2H), 2.50-2.39 (m, 2H), 0.83-0.72 (m, 4H); MS (ES+) m/z 456.2 (M+1).

### Example 171

### Synthesis of N-[2-(3,3-difluoropyrrolidin-1-yl)-4-(2-fluorophenyl)-3-pyridyl]-2-(1-methylcyclopropoxy)pyrimidine-5-carboxamide

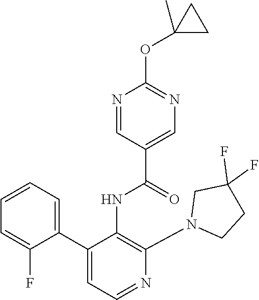 [SMILES: CC1(Oc2ncc(C(=O)Nc3c(-c4ccccc4F)ccnc3N3CCC(F)(F)C3)cn2)CC1]

To 2-chloro-N-[2-(3,3-difluoropyrrolidin-1-yl)-4-(2-fluorophenyl)-3-pyridyl]pyrimidine-5-carboxamide (0.10 g, 0.23 mmol) was added anhydrous 1-methyl-2-pyrrolidinone (0.50 mL) and 1-methylcyclopropan-1-ol (0.083 g, 1.2 mmol). Sodium hydride, 60% dispersion in mineral oil, (0.046 g, 1.2 mol), was added, the vial was sealed, and heated to 50° C. for 1 h. The reaction mixture was diluted with ethyl acetate 10 (200 mL), washed with saturated ammonium chloride solution (2×50 mL), dried over anhydrous magnesium sulfate, filtered and concentrated in vacuo Purification of the residue by preparative HPLC, eluting with a gradient of 15 to 85% acetonitrile in water containing 0.5% formic acid, provided the title compound as a colorless solid (0.0072 g, 6% yield): ¹H-NMR (400 MHz; DMSO-d₆) δ 10.07 (s, 1H), 8.79 (q, J=3.0 Hz, 2H), 8.20 (d, J=5.0 Hz, 1H), 7.40-7.34 (m, 1H), 7.34-7.28 (m, 1H), 7.28-7.22 (m, 1H), 7.21-7.17 (m, 1H), 6.81-6.80 (m, 1H), 3.96-3.82 (m, 2H), 3.82-3.70 (m, 2H), 2.50-2.39 (m, 3H), 1.60 (s, 3H), 0.94-0.90 (m, 2H), 0.79-0.75 (m, 2H); MS (ES+) m/z 470.2 (M+1).

### Example 172

### Synthesis of N-[2-(3,3-difluoropyrrolidin-1-yl)-4-(2-fluorophenyl)-3-pyridyl]-2-ethoxy-pyrimidine-5-carboxamide

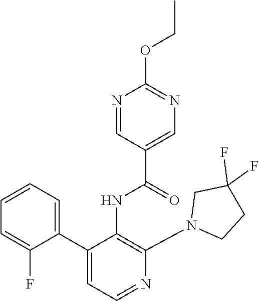 [SMILES: CCOc1ncc(C(=O)Nc2c(-c3ccccc3F)ccnc2N2CCC(F)(F)C2)cn1]

To 2-chloro-N-[2-(3,3-difluoropyrrolidin-1-yl)-4-(2-fluorophenyl)-3-pyridyl]pyrimidine-5-carboxamide (0.10 g, 0.23 mmol) was added anhydrous ethanol (0.50 mL). Sodium hydride, 60% dispersion in mineral oil, (0.046 g, 1.2 mol), was added, the vial was sealed, and heated to 50° C. for 1 h. The reaction mixture was diluted with ethyl acetate (200 mL), washed with saturated ammonium chloride solution (2×50 mL), dried over anhydrous magnesium sulfate, filtered and concentrated in vacuo Purification of the residue by preparative HPLC, eluting with a gradient of 15 to 85% acetonitrile in water containing 0.5% formic acid, provided the title compound as a colorless solid (0.0072 g, 6% yield): ¹H-NMR (400 MHz; DMSO-d₆) δ 510.03 (s, 1H), 8.79 (s, 2H), 8.21 (d, J=5.0 Hz, 1H), 7.36 (dddd, J=7.9, 5.4, 5.1, 2.4 Hz, 1H), 7.32-7.28 (m, 1H), 7.24 (ddd, J=10.0, 8.6, 1.1 Hz, 1H), 7.18 (td, J=7.5, 1.1 Hz, 1H), 6.81 (d, J=4.9 Hz, 1H), 4.40 (q, J=7.1 Hz, 2H), 3.93-3.86 (m, 2H), 3.76-3.72 (m, 2H), 2.47-2.38 (m, 2H), 1.33 (t, J=8.8 Hz, 3H); MS (ES+) m/z 444.2 (M+1).

### Example 173

### Synthesis of 2-(azetidin-1-yl)-N-[2-(3,3-difluoropyrrolidin-1-yl)-4-(2-fluorophenyl)-3-pyridyl]pyrimidine-5-carboxamide

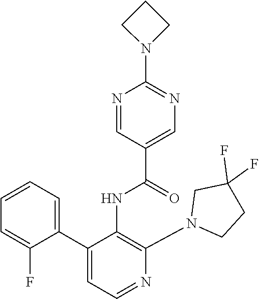 [SMILES: O=C(Nc1c(-c2ccccc2F)ccnc1N1CCC(F)(F)C1)c1cnc(N2CCC2)nc1]

To azetidine hydrochloride (0.013 g, 0.14 mmol) was added water (0.43 mL) and tetrahydrofuran (1.9 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.030 g, 0.069 mmol) was added dropwise and the reaction mixture was stirred for 1 h. The reaction mixture was diluted with ethyl acetate (25 mL), washed with saturated ammonium chloride solution (10 mL), dried over anhydrous magnesium sulfate, filtered and concentrated in vacuo. The solids were triturated with diethyl ether (5 mL) and filtered to provide the title compound as a colorless solid (0.024 g, 73% yield): ¹H-NMR (400 MHz; DMSO-d₆) δ 9.70 (s, 1H), 8.56 (d, J=5.8 Hz, 2H), 8.18 (d, J=5.0 Hz, 1H), 7.38-7.32 (m, 1H), 7.28 (td, J=7.6, 1.6 Hz, 1H), 7.26-7.20 (m, 1H), 7.16 (td, J=7.5, 1.1 Hz, 1H), 6.79 (dd, J=5.0, 0.5 Hz, 1H), 4.09 (t, J=7.6 Hz, 4H), 3.93-3.82 (m, 2H), 3.80-3.70 (m, 2H), 2.44 (ddt, J=21.1, 14.0, 6.9 Hz, 2H), 2.36-2.28 (m, 2H); MS (ES+) m/z 455.2 (M+1).

### Example 174

### Synthesis of N-[2-(3,3-difluoropyrrolidin-1-yl)-4-(2-fluorophenyl)-3-pyridyl]-2-(6-methoxy-2-azaspiro[3.3]heptan-2-yl)pyrimidine-5-carboxamide

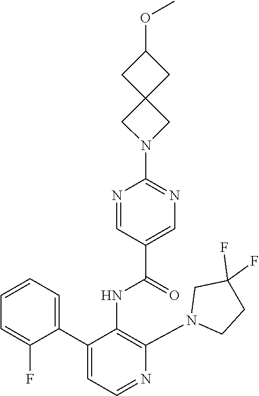 [SMILES: COC1CC2(C1)CN(c1ncc(C(=O)Nc3c(-c4ccccc4F)ccnc3N3CCC(F)(F)C3)cn1)C2]

To 6-methoxy-2-azasprio[3,3]heptane hydrochloride (0.023 g, 0.14 mmol) was added water (0.43 mL) and tetrahydrofuran (1.9 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.030 g, 0.069 mmol) was added dropwise and the reaction mixture was stirred for 1 h. The reaction mixture was diluted with ethyl acetate (25 mL), washed with saturated ammonium chloride solution (10 mL), dried over anhydrous magnesium sulfate, filtered and concentrated in vacuo. The solids were triturated with diethyl ether (5 mL) and filtered to provide the title compound as a colorless solid (0.030 g, 81% yield): ¹H-NMR (400 MHz; DMSO-d₆) δ 9.70 (s, 1H), 8.55 (s, 2H), 8.18 (d, J=5.0 Hz, 1H), 7.36-7.32 (m, 1H), 7.28 (td, J=7.6, 1.6 Hz, 1H), 7.22 (ddd, J=10.0, 8.6, 1.2 Hz, 1H), 7.16 (td, J=7.5, 1.1 Hz, 1H), 6.78 (dd, J=5.0, 0.6 Hz, 1H), 4.08 (s, 2H), 4.03 (s, 2H), 3.91 (s, 2H), 3.81-3.72 (m, 3H), 3.12 (s, 3H), 2.48 (dd, J=6.8, 3.0 Hz, 2H), 2.42 (dd, J=14.3, 7.0 Hz, 2H), 2.06 (ddd, J=9.9, 7.0, 2.9 Hz, 2H); MS (ES+) m/z 525.2 (M+1).

### Example 175

### Synthesis of N-[2-(3,3-difluoropyrrolidin-1-yl)-4-(2-fluorophenyl)-3-pyridyl]-2-(3-methoxyazetidin-1-yl)pyrimidine-5-carboxamide

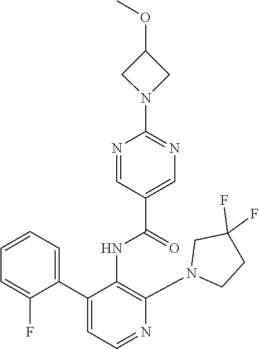 [SMILES: COC1CN(c2ncc(C(=O)Nc3c(-c4ccccc4F)ccnc3N3CCC(F)(F)C3)cn2)C1]

To 3-methoxyazetidine hydrochloride (0.017 g, 0.14 mmol) was added water (0.43 mL) and tetrahydrofuran (1.9 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.030 g, 0.069 mmol) was added dropwise and the reaction mixture was stirred for 1 h. The reaction mixture was diluted with ethyl acetate (25 mL), washed with saturated ammonium chloride solution (10 mL), dried over anhydrous magnesium sulfate, filtered and concentrated in vacuo. The solids were triturated with diethyl ether (5 mL) and filtered to provide the title compound as a colorless solid (0.022 g, 65% yield): ¹H-NMR (400 MHz; DMSO-d₆) δ 9.73 (s, 1H), 8.58 (s, 2H), 8.18 (d, J=5.0 Hz, 1H), 7.38-7.32 (m, 1H), 7.28 (td, J=7.6, 1.7 Hz, 1H), 7.23 (ddd, J=10.0, 8.6, 1.2 Hz, 1H), 7.16 (td, J=7.5, 1.1 Hz, 1H), 6.80-6.78 (m, 1H), 4.33-4.25 (m, 3H), 3.93-3.84 (m, 4H), 3.75-3.73 (m, 2H), 3.25 (s, 3H), 2.44 (td, J=14.2, 7.1 Hz, 2H); MS (ES+) m/z 485.2 (M+1).

### Example 176

### Synthesis of N-[2-(3,3-difluoropyrrolidin-1-yl)-4-(2-fluorophenyl)-3-pyridyl]-2-(6-oxa-1-azaspiro[3.3]heptan-1-yl)pyrimidine-5-carboxamide

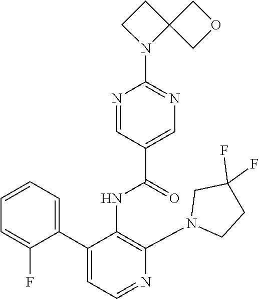 [SMILES: O=C(Nc1c(-c2ccccc2F)ccnc1N1CCC(F)(F)C1)c1cnc(N2CCC23COC3)nc1]

To 6-oxa-1-azaspiro[3,3]heptane hemioxalate (0.066 g, 0.23 mmol) was added water (0.71 mL) and tetrahydrofuran (3.2 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.050 g, 0.12 mmol) was added dropwise and the reaction mixture was stirred for 1 h. The reaction mixture was diluted with ethyl acetate (50 mL), washed with saturated ammonium chloride solution (2×20 mL), dried over anhydrous magnesium sulfate, filtered and concentrated in vacuo. Purification by column chromatography, eluting with a gradient of 20 to 100% ethyl acetate in heptane, provided the title compound as a colorless solid (0.027 g, 46% yield): ¹H-NMR (400 MHz; DMSO-d₆) δ b9.76 (s, 1H), 8.67-8.60 (m, 2H), 8.19 (d, J=5.0 Hz, 1H), 7.39-7.33 (m, 1H), 7.32-7.28 (m, 1H), 7.24 (ddd, J=10.0, 8.6, 1.2 Hz, 1H), 7.18 (td, J=7.5, 1.1 Hz, 1H), 6.79 (dd, J=4.9, 0.5 Hz, 1H), 5.29 (d, J=7.1 Hz, 2H), 4.54 (d, J=7.1 Hz, 2H), 3.96-3.82 (m, 4H), 3.81-3.69 (m, 2H), 2.64-2.61 (m, 2H), 2.50-2.39 (m, 2H); MS (ES+) m/z 497.2 (M+1).

### Example 177

### Synthesis of N-[2-(3,3-difluoropyrrolidin-1-yl)-4-(2-fluorophenyl)-3-pyridyl]-2-(1-oxa-6-azaspiro[3.3]heptan-6-yl)pyrimidine-5-carboxamide

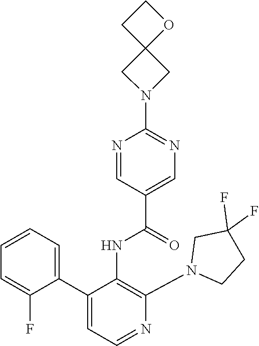 [SMILES: O=C(Nc1c(-c2ccccc2F)ccnc1N1CCC(F)(F)C1)c1cnc(N2CC3(CCO3)C2)nc1]

To 1-oxa-6-azaspiro[3,3]heptane hemioxalate (0.066 g, 0.23 mmol) was added water (0.71 mL) and tetrahydrofuran (3.2 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.050 g, 0.12 mmol) was added dropwise and the reaction mixture was stirred for 1 h. The reaction mixture was diluted with ethyl acetate (50 mL), washed with saturated ammonium chloride solution (2×20 mL), dried over anhydrous magnesium sulfate, filtered and concentrated in vacuo. Purification by column chromatography, eluting with a gradient of 20 to 100% ethyl acetate in heptane, provided the title compound as a colorless solid (0.031 g, 52% yield): ¹H-NMR (400 MHz; DMSO-d₆) δ 9.74 (s, 1H), 8.57 (s, 2H), 8.18 (d, J=5.0 Hz, 1H), 7.38-7.32 (m, 1H), 7.28 (td, J=7.6, 1.6 Hz, 1H), 7.26-7.20 (m, 1H), 7.16 (td, J=7.5, 1.1 Hz, 1H), 6.79 (d, J=4.9 Hz, 1H), 4.44 (t, J=7.5 Hz, 2H), 4.34 (dd, J=10.9, 1.6 Hz, 2H), 4.17 (dd, J=10.9, 1.6 Hz, 2H), 3.92-3.82 (m, 2H), 3.79-3.71 (m, 2H), 2.87 (t, J=7.5 Hz, 2H), 2.48-2.37 (m, 2H); MS (ES+) m/z 497.2 (M+1).

### Example 178

### Synthesis of N-[2-(3,3-difluoropyrrolidin-1-yl)-4-(2-fluorophenyl)-3-pyridyl]-2-(2-methylazetidin-1-yl)pyrimidine-5-carboxamide

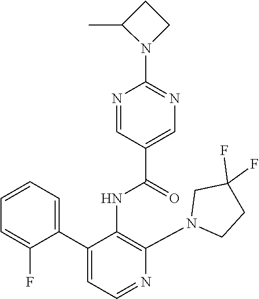 [SMILES: CC1CCN1c1ncc(C(=O)Nc2c(-c3ccccc3F)ccnc2N2CCC(F)(F)C2)cn1]

To 2-methylazetidine hydrochloride (0.025 g, 0.23 mmol) was added water (0.71 mL) and tetrahydrofuran (3.2 mL). While stirring at a high rate, 10 M sodium hydroxide solution (0.035 mL) was added dropwise. A tetrahydrofuran (0.35 mL) solution of 2-chloro-N-[2-(3,3-difluoropyrrolidin-1-yl)-4-(2-fluorophenyl)-3-pyridyl]pyrimidine-5-carboxamide (0.050 g, 0.12 mmol) was added dropwise and the reaction mixture was stirred for 1 h. The reaction mixture was diluted with ethyl acetate (100 mL), washed with saturated ammonium chloride solution (2×50 mL), dried over anhydrous magnesium sulfate, filtered and concentrated in vacuo. Purification by column chromatography, eluting with a gradient of 10 to 100% ethyl acetate in heptane, followed by trituration with diethyl ether (10 mL), provided the title compound as a colorless solid (0.048 g, 88% yield): ¹H-NMR (400 MHz; DMSO-d₆) δ 9.69 (s, 1H), 8.56 (s, 2H), 8.18 (d, J=5.0 Hz, 1H), 7.38-7.33 (m, 1H), 7.28 (td, J=7.5, 1.6 Hz, 1H), 7.26-7.21 (m, 1H), 7.17 (td, J=7.5, 1.1 Hz, 1H), 6.79 (dd, J=4.9, 0.3 Hz, 1H), 4.47 (dt, J=8.0, 6.2 Hz, 1H), 4.06-3.82 (m, 4H), 3.79-3.70 (m, 2H), 2.50-2.38 (m, 3H), 1.99-1.91 (m, 1H), 1.44 (d, J=6.3 Hz, 3H); MS (ES+) m/z 469.2 (M+1).

### Example 179

### Synthesis of N-[2-(3,3-difluoropyrrolidin-1-yl)-4-(2-fluorophenyl)-3-pyridyl]indane-2-carboxamide

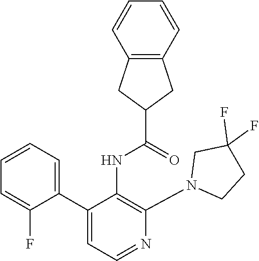 [SMILES: O=C(Nc1c(-c2ccccc2F)ccnc1N1CCC(F)(F)C1)C1Cc2ccccc2C1]

To a solution of 2-(3,3-difluoropyrrolidin-1-yl)-4-(2-fluorophenyl)pyridin-3-amine (0.10 g, 0.34 mmol), 2-indanecarboxylic acid (0.11 g, 0.68 mmol) and 2-chloro-1-methylpyridinium iodide (0.22 g, 0.85 mmol) was added anhydrous tetrahydrofuran (3.4 mL). The solution was heated to 50° C. for 2 min before N-ethyl-N-isopropylpropan-2-amine (0.44 g, 3.4 mmol) was added. The reaction mixture was heated to 50° C. for 3 h. The reaction mixture was cooled to ambient temperature, diluted with ethyl acetate (20 mL), washed with saturated ammonium chloride solution (10 mL), dried over anhydrous magnesium sulfate, filtered and concentrated in vacuo. Purification by column chromatography, eluting with a gradient of 10 to 100% ethyl acetate in heptane, followed by trituration with a 1:1 mixture of diethyl ether: heptane (10 mL) provided the title compound as a colorless solid (0.066 g, 73% yield): ¹H-NMR (400 MHz; DMSO-d₆) δ 9.45 (s, 1H), 8.15 (d, J=4.9 Hz, 1H), 7.49-7.44 (m, 1H), 7.32-7.28 (m, 1H), 7.27-7.20 (m, 2H), 7.12-7.06 (m, 4H), 6.74 (d, J=4.9 Hz, 1H), 3.97-3.83 (m, 2H), 3.83-3.71 (m, 2H), 3.19-3.10 (m, 1H), 2.89-2.76 (m, 2H), 2.76-2.54 (m, 2H), 2.49-2.41 (m, 2H); MS (ES+) m/z 438.2 (M+1).

### Example 180

### Synthesis of N-[2-(3,3-difluoropyrrolidin-1-yl)-4-(2-fluorophenyl)-3-pyridyl]-2,3-dihydrobenzofuran-2-carboxamide

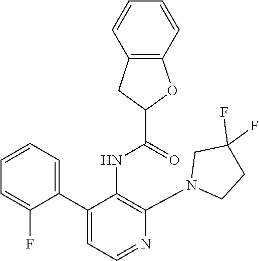 [SMILES: O=C(Nc1c(-c2ccccc2F)ccnc1N1CCC(F)(F)C1)C1Cc2ccccc2O1]

To a solution of 2-(3,3-difluoropyrrolidin-1-yl)-4-(2-fluorophenyl)pyridin-3-amine (0.10 g, 0.34 mmol), 2,3-dihydro-1-benzofuran-2-carboxylic acid (0.11 g, 0.68 mmol) and 2-chloro-1-methylpyridinium iodide (0.22 g, 0.85 mmol) was added anhydrous tetrahydrofuran (3.4 mL). The solution was heated to 50° C. for 2 min before N-ethyl-N-isopropylpropan-2-amine (0.44 g, 3.4 mmol) was added. The reaction mixture was heated to 50° C. for 3 h. The reaction mixture was cooled to ambient temperature, diluted with ethyl acetate (20 mL), washed with saturated ammonium chloride solution (10 mL), dried over anhydrous magnesium sulfate, filtered and concentrated in vacuo.

Purification by column chromatography, eluting with a gradient of 15 to 100% ethyl acetate in heptane, followed by trituration with diethyl ether (10 mL) provided the title compound as a colorless solid (0.016 g, 11% yield): ¹H-NMR (400 MHz; DMSO-d₆) δ 9.64-9.60 (m, 1H), 8.13 (t, J=4.8 Hz, 1H), 7.36-7.31 (m, 1H), 7.21-7.17 (m, 1H), 7.17-7.05 (m, 3H), 7.05-6.99 (m, 1H), 6.86 (dd, J=9.4, 5.4 Hz, 1H), 6.83 (t, J=7.6 Hz, 1H), 6.72 (d, J=4.9 Hz, 1H), 5.02 (dt, J=7.9, 4.1 Hz, 1H), 3.90-3.77 (m, 2H), 3.74-3.63 (m, 2H), 3.31-3.23 (m, 1H), 2.60-2.53 (m, 1H), 2.43-2.31 (m, 2H); MS (ES+) m/z 440.2 (M+1).

### Example 181

### Synthesis of (S)-N-(4-(2,5-difluorophenyl)-2-(3-fluoropyrrolidin-1-yl)pyridin-3-yl)-4-methylpentanamide

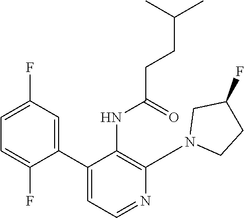 [SMILES: CC(C)CCC(=O)Nc1c(-c2cc(F)ccc2F)ccnc1N1CC[C@H](F)C1]

### Step 1. Preparation of 2-chloro-4-(2,5-difluorophenyl)-3-nitropyridine

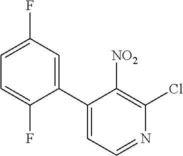 [SMILES: O=[N+]([O-])c1c(-c2cc(F)ccc2F)ccnc1Cl]

A mixture of 2,4-dichloro-3-nitropyridine(2.00 g, 10.4 mmol), 1,4-dioxane (20 mL), and water (4 mL) was sparged with nitrogen for 10 min. To this solution was added potassium carbonate (3.15 g, 22.8 mmol), 2,5-difluoroboronic acid (1.64 g, 10.4 mmol), and [1,1′-bis(diphenylphosphino)ferrocene]dichloropalladium(II), complex with dichloromethane (0.888 g, 1.04 mmol). The solution was heated at 60° C. under nitrogen for 2 h. The reaction mixture was cooled to ambient temperature, filtered through diatomaceous earth (i.e., Celite®), and concentrated in vacuo. The residue was purified by column chromatography, eluting with 0-60% ethyl acetate in heptane, affording the title compound as a yellow solid (2.05 g, 73% yield): ¹H-NMR (300 MHz; CDCl₃): 58.62 (d, J=5.0 Hz, 1H), 8.46 (d, J=5.3 Hz, 1H), 7.49 (d, J=5.1 Hz, 1H), 7.41 (dd, J=5.1, 1.2 Hz, 1H), 7.22-7.17 (m, 2H), 7.04 (dddd, J=7.9, 5.7, 2.0, 1.5 Hz, 1H).

### Step 2. Preparation of (S)-4-(2,5-difluorophenyl)-2-(3-fluoropyrrolidin-1-yl)-3-nitropyridine

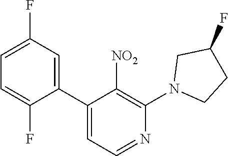 [SMILES: O=[N+]([O-])c1c(-c2cc(F)ccc2F)ccnc1N1CC[C@H](F)C1]

To a solution of 2-chloro-4-(2,5-difluorophenyl)-3-nitropyridine (2.0 g, 7.5 mmol) in anhydrous N,N-dimethylformamide (25 mL) was added (S)-3-fluoropyrrolidine hydrochloride (1.0 g 8.3 mmol) and N-ethyl-N-isopropylpropan-2-amine (2.90 g, 27.5 mmol) at ambient temperature. The mixture was stirred at ambient temperature for 16 h. The resulting mixture was diluted with ethyl acetate (300 mL). The organic layer was washed with saturated ammonium chloride (100 mL), water (100 mL), and brine (100 mL). The organic layer was dried over anhydrous magnesium sulfate, filtered, and concentrated in vacuo. The residue was purified by column chromatography, eluting with a gradient of 0 to 60% ethyl acetate in heptane, to afford the title compound as a beige solid (1.37 g, 57% yield): MS (ESI+) 324 m/z (M+1).

### Step 3. Preparation of (S)-4-(2,5-difluorophenyl)-2-(3-fluoropyrrolidin-1-yl)pyridin-3-amine

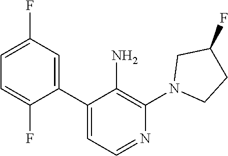 [SMILES: Nc1c(-c2cc(F)ccc2F)ccnc1N1CC[C@H](F)C1]

To a solution of (S)-4-(2,5-difluorophenyl)-2-(3-fluoropyrrolidin-1-yl)-3-nitropyridine (1.4 g, 4.2 mmol) in acetic acid (42.4 mL) was added elemental iron (0.60 g, 11 mmol) and stirred at 60° C. for 1 h. The reaction mixture was charged with elemental iron (0.60 g, 11 mmol) and stirred at 60° C. for 1 h. The mixture was cooled to ambient temperature and filtered, rinsing with ethyl acetate (3×50 mL). The filtrate was concentrated in vacuo. The residue was dissolved in ethyl acetate (250 mL) and washed with saturated ammonium chloride (50 mL), and brine (50 mL). The organic layer was dried over anhydrous magnesium sulfate, filtered, and concentrated in vacuo. The residue was purified by column chromatography, eluting with a gradient of 0 to 100% ethyl acetate in heptane to afford the title compound as a yellow oil (0.5 g, 41% yield): MS (ESI+) 294 m/z (M+1).

### Step 4. Preparation of (S)-N-(4-(2,5-difluorophenyl)-2-(3-fluoropyrrolidin-1-yl)pyridin-3-yl)-4-methylpentanamide

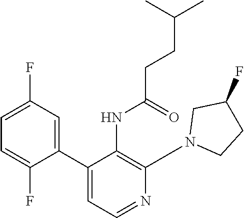 [SMILES: CC(C)CCC(=O)Nc1c(-c2cc(F)ccc2F)ccnc1N1CC[C@H](F)C1]

To a solution of (S)-4-(2,5-difluorophenyl)-2-(3-fluoropyrrolidin-1-yl)pyridin-3-amine (0.050 g, 0.17 mmol), and 4-methylpentanoic acid (0.018 g, 0.15 mmol) in N,N-dimethylformamide (0.2 mL), was added pyridine (0.1 mL) at ambient temperature. The mixture was then cooled to −10° C. A 50% ethyl acetate solution of 2,4,6-tripropyl-1,3,5,2,4,6-trioxatriphosphinane 2,4,6-trioxide (0.3 mL) was added. The mixture was allowed to warm to ambient temperature and stirred for 16 h. The reaction mixture was diluted with ethyl acetate (100 mL). The organic layer was washed with saturated ammonium chloride (20 mL), water (20 mL), and brine (20 mL). The organic layer was dried over anhydrous magnesium sulfate, filtered, and concentrated in vacuo. The residue was purified by column chromatography, eluting with a gradient of 0 to 100% ethyl acetate in heptane, followed by 10 to 50% methanol in dichloromethane to afford the title compound as a colorless solid (0.017 g, 31% yield): ¹H-NMR (300 MHz; DMSO-d₆): δ 9.30 (s, 1H), 8.09 (d, J=4.9 Hz, 1H), 7.34-7.25 (m, 2H), 7.07-7.01 (m, 1H), 6.63 (dd, J=4.9, 0.8 Hz, 1H), 5.46-5.28 (m, 1H), 3.71-3.61 (m, 4H), 2.19-1.99 (m, 4H), 1.11-1.04 (m, 3H), 0.73-0.71 (m, 6H); MS (ESI+) 392.1 m/z (M+1).

### Example 182

### Synthesis of (S)-6-chloro-N-(4-(2,5-difluorophenyl)-2-(3-fluoropyrrolidin-1-yl)pyridin-3-yl)nicotinamide

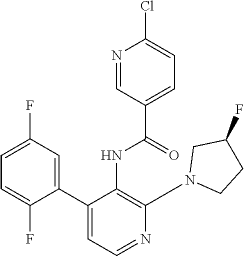 [SMILES: O=C(Nc1c(-c2cc(F)ccc2F)ccnc1N1CC[C@H](F)C1)c1ccc(Cl)nc1]

### Step 1. Preparation of (S)-6-chloro-N-(4-(2,5-difluorophenyl)-2-(3-fluoropyrrolidin-1-yl)pyridin-3-yl)nicotinamide

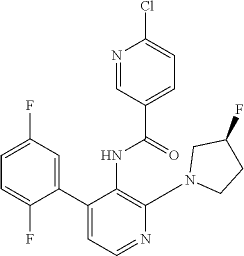 [SMILES: O=C(Nc1c(-c2cc(F)ccc2F)ccnc1N1CC[C@H](F)C1)c1ccc(Cl)nc1]

To a solution of (S)-4-(2,5-difluorophenyl)-2-(3-fluoropyrrolidin-1-yl)pyridin-3-amine (0.30 g, 1.0 mmol), and 6-chloronicotinic acid (0.15 g, 0.93 mmol) in N,N-dimethylformamide (0.7 mL), was added pyridine (0.5 mL) at ambient temperature. The mixture was then cooled to −10 0° C. A 50% ethyl acetate solution of 2,4,6-tripropyl-1,3,5,2,4,6-trioxatriphosphinane 2,4,6-trioxide (0.9 mL) was added. The mixture was allowed to warm to ambient temperature and stirred for 16 h. The reaction mixture was diluted with ethyl acetate (100 mL). The organic layer was washed with saturated ammonium chloride (20 mL), water (20 mL), and brine (20 mL). The organic layer was dried over anhydrous magnesium sulfate, filtered, and concentrated in vacuo. The residue was purified by column chromatography, eluting with a gradient of 0 to 60% ethyl acetate in heptane, followed by purification by preparative reverse phase HPLC, using acetonitrile in water containing 0.1% of trifluoroacetic acid as eluent, afforded the title compound as a colorless solid (0.122 g, 28% yield): ¹H-NMR (300 MHz; DMSO-d₆): δ 10.22-10.16 (m, 1H), 8.66 (dd, J=2.5, 0.7 Hz, 1H), 8.19-8.17 (m, 1H), 8.09-8.06 (m, 1H), 7.65 (dd, J=8.3, 0.7 Hz, 1H), 7.33-7.10 (m, 3H), 6.74-6.72 (m, 1H), 5.44-5.25 (m, 1H), 3.85-3.58 (m, 4H), 2.18-1.94 (m, 2H); MS (ESI+) 433.1 m/z (M+1), 435.1 m/z (M+1).

### Example 183

### Synthesis of (S)-N-(2-(3-fluoropyrrolidin-1-yl)-4-phenylpyridin-3-yl)-6-isopropylnicotinamide

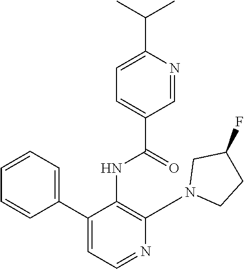 [SMILES: CC(C)c1ccc(C(=O)Nc2c(-c3ccccc3)ccnc2N2CC[C@H](F)C2)cn1]

### Step 1. Preparation of 2-chloro-3-nitro-4-phenylpyridine

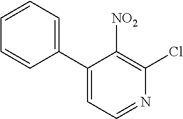 [SMILES: O=[N+]([O-])c1c(-c2ccccc2)ccnc1Cl]

A mixture of 2,4-dichloro-3-nitropyridine (7.09 g, 36.7 mmol), 1,4-dioxane (71 mL), and water (24 mL) was sparged with nitrogen for 10 min. The flask was charged with phenylboronic acid (4.5 g, 37 mmol), potassium carbonate (7.6 g, 55 mmol), and [1,1′-bis(diphenylphosphino)ferrocene]dichloropalladium(II), complex with dichloromethane (3.1 g, 3.7 mmol), and sparged for 2 min. The reaction mixture was stirred at 80° C. for 2 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 5-60% ethyl acetate in heptane, afforded the title compound as a colorless oil that solidified upon standing (6.0 g, 70% yield): ¹H-NMR (300 MHz; CDCl₃): δ 58.57 (d, J=5.1 Hz, 1H), 7.53-7.49 (m, 3H), 7.43-7.38 (m, 3H).

### Step 2. Preparation of (S)-2-(3-fluoropyrrolidin-1-yl)-3-nitro-4-phenylpyridine

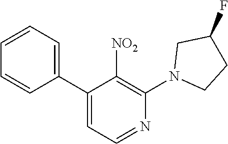 [SMILES: O=[N+]([O-])c1c(-c2ccccc2)ccnc1N1CC[C@H](F)C1]

To a mixture of 2-chloro-3-nitro-4-phenylpyridine (2.03 g, 8.65 mmol), anhydrous potassium carbonate (3.59 g, 26.0 mmol), and (S)-3-fluoropyrrolidine hydrochloride was added N,N-dimethylformamide (28.8 mL). The flask was sealed and heated to 50° C. for 16 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 5-60% ethyl acetate in heptane, afforded the title compound as a yellow oil (2.30 g, 92% yield): ¹H-NMR (300 MHz; CDCl₃): δ 8.28 (d, J=4.9 Hz, 1H), 7.46-7.41 (m, 3H), 7.36-7.32 (m, 2H), 6.63 (d, J=4.9 Hz, 1H), 5.44-5.24 (m, 1H), 3.87-3.72 (m, 2H), 3.70-3.62 (m, 2H), 2.38 (dddq, J=17.1, 14.0, 6.4, 1.6 Hz, 1H), 2.23-1.97 (m, 1H).

### Step 3. Preparation of (S)-2-(3-fluoropyrrolidin-1-yl)-4-phenylpyridin-3-amine

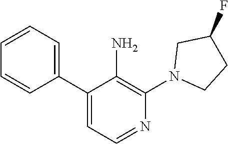 [SMILES: Nc1c(-c2ccccc2)ccnc1N1CC[C@H](F)C1]

To a mixture of (S)-2-(3-fluoropyrrolidin-1-yl)-3-nitro-4-phenylpyridine (2.30 g, 8.01 mmol) was added anhydrous methanol (20 mL), and 10% palladium on carbon (0.23 g). The reaction vessel was sealed and the reaction mixture was sparged with hydrogen gas for 5 min. The reaction was stirred under a hydrogen atmosphere for 24 h. The reaction mixture was filtered through diatomaceous earth (i.e., Celite®), washed with methanol (5×20 mL) and concentrated in vacuo. The resulting brown oil was used as is (1.54 g, 75% yield): ¹H-NMR (300 MHz; CDCl₃): 57.81 (d, J=5.1 Hz, 1H), 7.54-7.49 (m, 4H), 7.49-7.39 (m, 1H), 6.81 (d, J=5.0 Hz, 1H), 5.50-5.28 (m, 1H), 3.84-3.80 (m, 2H), 3.79-3.70 (m, 2H), 3.63 (d, J=3.6 Hz, 1H), 3.36 (ddd, J=10.1, 7.4, 5.9 Hz, 1H), 2.31 (td, J=7.1, 3.6 Hz, 1H), 2.24-2.18 (m, 1H).

### Step 4. Preparation of (S)-N-(2-(3-fluoropyrrolidin-1-yl)-4-phenylpyridin-3-yl)-6-isopropylnicotinamide

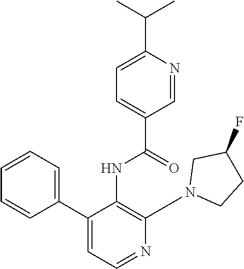 [SMILES: CC(C)c1ccc(C(=O)Nc2c(-c3ccccc3)ccnc2N2CC[C@H](F)C2)cn1]

To a mixture of (S)-2-(3-fluoropyrrolidin-1-yl)-4-phenylpyridin-3-amine (0.10 g, 0.39 mmol) and isopropylnicotinic acid hydrochloride (0.078 g, 0.39 mmol) was added N,N-dimethylformamide (1 mL) and anhydrous pyridine (0.5 mL). A 50% solution of propylphosphonic anhydride in N,N-dimethylformamide (1 mL) was added and the reaction mixture was stirred at ambient temperature for 4 h. Isopropylnicotinic acid hydrochloride (0.078 g, 0.39 mmol) and a 50% solution of propylphosphonic anhydride in N,N-dimethylformamide (1 mL) was added and the reaction mixture was stirred for 14 h. The reaction mixture was diluted with ethyl acetate (200 mL) and washed with saturated ammonium chloride solution (2×50 mL). The organic layer was dried over anhydrous magnesium sulfate, filtered and concentrated in vacuo. The residue was purified by preparative HPLC eluting with a gradient of 10 to 75% of acetonitrile in water containing 0.5% formic acid to provide the title compound as a colorless solid (0.029 g, 18% yield): ¹H-NMR (300 MHz; DMSO-d₆): δ 10.01-9.89 (m, 1H), 8.77-8.76 (m, 1H), 8.15-8.13 (m, 1H), 7.96 (dd, J=8.2, 2.3 Hz, 1H), 7.40-7.26 (m, 6H), 6.68 (d, J=5.0 Hz, 1H), 5.34 (d, J=54.0 Hz, 1H), 3.90-3.54 (m, 4H), 3.05 (dt, J=13.8, 6.9 Hz, 1H), 2.17-1.92 (m, 2H), 1.19 (d, J=0.5 Hz, 6H); MS (ES+) m/z 405.2 (M+1).

### Example 184

### Synthesis of N-(2-(3,3-difluoropyrrolidin-1-yl)-4-phenylpyridin-3-yl)-6-isopropylnicotinamide

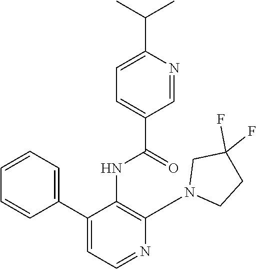 [SMILES: CC(C)c1ccc(C(=O)Nc2c(-c3ccccc3)ccnc2N2CCC(F)(F)C2)cn1]

### Step 1. Preparation of 2-(3,3-difluoropyrrolidin-1-yl)-3-nitro-4-phenylpyridine

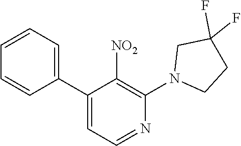 [SMILES: O=[N+]([O-])c1c(-c2ccccc2)ccnc1N1CCC(F)(F)C1]

To a mixture of 2-chloro-3-nitro-4-phenylpyridine (1.48 g, 6.31 mmol), anhydrous potassium carbonate (2.62 g, 18.9 mmol), and 3,3-difluoropyrrolidine hydrochloride (1.18 g, 8.20) was added N,N-dimethylformamide (21 mL). The flask was sealed and heated to 70° C. for 16 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 5-60% ethyl acetate in heptane, afforded the title compound as a yellow solid (1.7 g, 88% yield).

### Step 2. Preparation of 2-(3,3-difluoropyrrolidin-1-yl)-4-phenylpyridin-3-amine

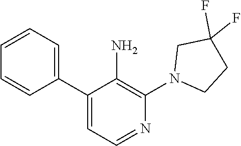 [SMILES: Nc1c(-c2ccccc2)ccnc1N1CCC(F)(F)C1]

To a mixture of 2-(3,3-difluoropyrrolidin-1-yl)-3-nitro-4-phenylpyridine (1.7 g, 5.6 mmol) was added anhydrous methanol (22 mL) and 10% palladium on carbon (0.59 g). The reaction vessel was sealed and the reaction mixture was sparged with hydrogen gas for 5 min. The reaction was stirred under a hydrogen atmosphere for 24 h. The reaction mixture was filtered through diatomaceous earth (i.e., Celite®), washed with methanol (5×20 mL) and concentrated in vacuo. The resulting brown oil was used as is (1.4 g, 91% yield): ¹H-NMR (300 MHz; CDCl₃): δ 7.84 (d, J=5.0 Hz, 1H), 7.52-7.48 (m, 4H), 7.46-7.40 (m, 1H), 6.87 (d, J=5.0 Hz, 1H), 3.86 (s, 2H), 3.73 (t, J=13.2 Hz, 2H), 3.58 (t, J=7.1 Hz, 2H), 2.47 (tt, J=14.4, 7.2 Hz, 2H).

### Step 3. Preparation of N-(2-(3,3-difluoropyrrolidin-1-yl)-4-phenylpyridin-3-yl)-6-isopropylnicotinamide

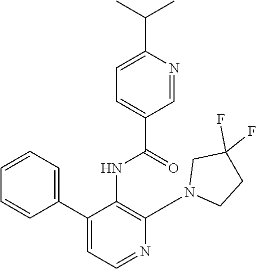 [SMILES: CC(C)c1ccc(C(=O)Nc2c(-c3ccccc3)ccnc2N2CCC(F)(F)C2)cn1]

To a mixture of 2-(3,3-difluoropyrrolidin-1-yl)-4-phenylpyridin-3-amine (0.10 g, 0.36 mmol) and isopropylnicotinic acid hydrochloride (0.073 g, 0.36 mmol) was added N,N-dimethylformamide (1.6 mL) and anhydrous pyridine (0.65 mL). A 50% solution of propylphosphonic anhydride in N,N-dimethylformamide (1.6 mL) was added and the reaction mixture was stirred at ambient temperature for 14 h. The reaction mixture was diluted with ethyl acetate (150 mL) and washed with saturated ammonium chloride solution (2×50 mL). The organic layer was dried over anhydrous magnesium sulfate, filtered and concentrated in vacuo. The residue was purified by preparative HPLC eluting with a gradient of 10 to 80% of acetonitrile in water containing 0.1% trifluoroacetic acid to provide the title compound as a colorless solid (0.024 g, 16% yield): ¹H-NMR (300 MHz; DMSO-d₆) δ 10.02 (s, 1H), 8.77 (d, J=1.9 Hz, 1H), 8.19 (d, J=5.0 Hz, 1H), 8.01 (dd, J=8.2, 2.3 Hz, 1H), 7.44-7.28 (m, 6H), 6.81 (d, J=5.0 Hz, 1H), 3.94-3.69 (m, 4H), 3.07 (dt, J=13.8, 6.9 Hz, 1H), 2.49-2.39 (m, 3H), 1.24 (t, J=5.6 Hz, 6H); MS (ES+) m/z 423.2 (M+1).

### Example 185

### Synthesis of (S)-N-(2-(3-fluoropyrrolidin-1-yl)-4-phenylpyridin-3-yl)-2-isopropylpyrimidine-5-carboxamide

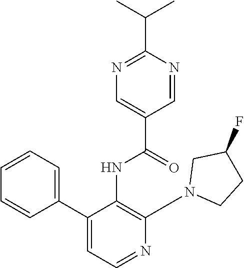 [SMILES: CC(C)c1ncc(C(=O)Nc2c(-c3ccccc3)ccnc2N2CC[C@H](F)C2)cn1]

### Step 1. Preparation of (S)-N-(2-(3-fluoropyrrolidin-1-yl)-4-phenylpyridin-3-yl)-2-isopropylpyrimidine-5-carboxamide

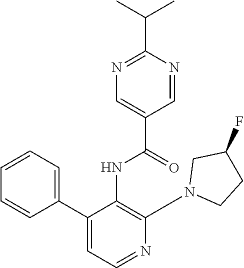 [SMILES: CC(C)c1ncc(C(=O)Nc2c(-c3ccccc3)ccnc2N2CC[C@H](F)C2)cn1]

To a mixture of (S)-2-(3-fluoropyrrolidin-1-yl)-4-phenylpyridin-3-amine (0.12 g, 0.45 mmol) and 2-isopropylpyrimidine-5-carboxylic acid (0.076 g, 0.45 mmol) was added N,N-dimethylformamide (1 mL) and anhydrous pyridine (0.5 mL). A 50% solution of propylphosphonic anhydride in N,N-dimethylformamide (1.8 mL) was added 20 and the reaction mixture was stirred at ambient temperature for 14 h. The reaction mixture was diluted with ethyl acetate (200 mL) and washed with saturated ammonium chloride solution (2×100 mL). The organic layer was dried over anhydrous magnesium sulfate, filtered and concentrated in vacuo. The residue was purified by preparative HPLC, eluting with a gradient of 10 to 60% of acetonitrile in water containing 0.1% trifluoroacetic acid, followed by preparative HPLC, eluting with a gradient of 10 to 60% of acetonitrile in water containing 0.5% formic acid, to provide the title compound as a colorless solid (0.07.2 g, 4% yield): ¹H-NMR (300 MHz; DMSO-d₆) δ 10.23-10.22 (m, 1H), 9.15 (s, 1H), 8.92 (s, 2H), 8.15 (d, J=5.3 Hz, 1H), 7.40-7.34 (m, 5H), 6.78-6.77 (m, 1H), 5.46-5.28 (m, 1H), 3.83-3.64 (m, 4H), 3.19 (dq, J=13.9, 7.0 Hz, 2H), 2.15-2.14 (m, 2H), 1.28 (td, J=7.6, 1.9 Hz, 1OH); MS (ES+) m/z 406.4 (M+1).

### Example 186

### Synthesis of N-(2-(3,3-difluoropyrrolidin-1-yl)-4-phenylpyridin-3-yl)-1-isopropyl-1H-pyrazole-4-carboxamide

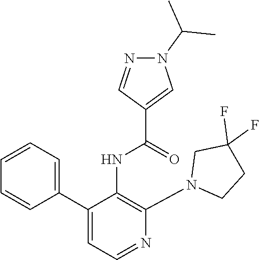 [SMILES: CC(C)n1cc(C(=O)Nc2c(-c3ccccc3)ccnc2N2CCC(F)(F)C2)cn1]

### Step 1. Preparation of N-(2-(3,3-difluoropyrrolidin-1-yl)-4-phenylpyridin-3-yl)-1-isopropyl-1H-pyrazole-4-carboxamide

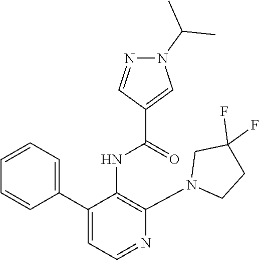 [SMILES: CC(C)n1cc(C(=O)Nc2c(-c3ccccc3)ccnc2N2CCC(F)(F)C2)cn1]

To a mixture of 2-(3,3-difluoropyrrolidin-1-yl)-4-phenylpyridin-3-amine (0.070 g, 0.25 mmol), 1-isopropyl-1H-pyrazole-4-carboxylic acid (0.060 g, 0.38 mmol) and 2-chloro-1-methylpyridinium iodide (0.25 g, 0.96 mmol) was added anhydrous tetrahydrofuran (3.9 mL). The solution was heated at 65° C. for 1 h before N-ethyl-N-isopropylpropan-2-amine (0.34 g, 1.9 mmol) was added. The reaction mixture was stirred at 65° C. for 14 h. The reaction mixture was cooled to ambient temperature and diluted with ethyl acetate (150 mL). The reaction mixture was washed with saturated ammonium chloride solution (2×50 mL). The organic layer was dried over anhydrous magnesium sulfate, filtered and concentrated in vacuo. The residue was purified by preparative HPLC, eluting with a gradient of 5 to 45% of acetonitrile in water containing 0.1% trifluoroacetic acid, to provide the title compound as a colorless solid (0.026 g, 32% yield): ¹H-NMR (300 MHz; DMSO-d₆) δ 9.41 (s, 1H), 8.15 (t, J=2.5 Hz, 2H), 7.84 (d, J=0.5 Hz, 1H), 7.41-7.30 (m, 5H), 6.76 (d, J=5.0 Hz, 1H), 4.49 (dt, J=13.3, 6.7 Hz, 1H), 3.92-3.68 (m, 4H), 2.49-2.35 (m, 3H), 1.38 (t, J=5.8 Hz, 6H); MS (ES+) m/z 412.3 (M+1).

### Example 187

### Synthesis of (S)-N-(2-(3-fluoropyrrolidin-1-yl)-4-phenylpyridin-3-yl)-1-isopropyl-1H-pyrazole-4-carboxamide

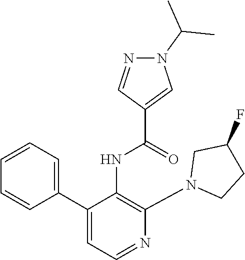 [SMILES: CC(C)n1cc(C(=O)Nc2c(-c3ccccc3)ccnc2N2CC[C@H](F)C2)cn1]

### Step 1. Preparation of (S)-N-(2-(3-fluoropyrrolidin-1-yl)-4-phenylpyridin-3-yl)-1-isopropyl-1H-pyrazole-4-carboxamide

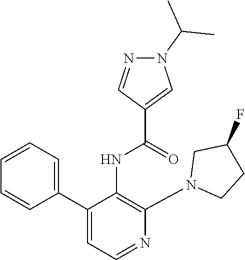 [SMILES: CC(C)n1cc(C(=O)Nc2c(-c3ccccc3)ccnc2N2CC[C@H](F)C2)cn1]

To a mixture of (S)-2-(3-fluoropyrrolidin-1-yl)-4-phenylpyridin-3-amine (0.093 g, 0.36 mmol), 1-isopropyl-1H-pyrazole-4-carboxylic acid (0.11 g, 0.73 mmol) and 2-chloro-1-methylpyridinium iodide (0.37 g, 1.5 mmol) was added anhydrous tetrahydrofuran (4.5 mL). The solution was heated at 65° C. for 5 min before N-ethyl-N-isopropylpropan-2-amine (0.47 g, 3.6 mmol) was added. The reaction mixture was stirred at 65° C. for 14 h. The reaction mixture was cooled to ambient temperature and diluted with ethyl acetate (200 mL). The reaction mixture was washed with saturated ammonium chloride solution (2×100 mL). The organic layer was dried over anhydrous magnesium sulfate, filtered and concentrated in vacuo. The residue was purified by preparative HPLC, eluting with a gradient of 5 to 35% of acetonitrile in water containing 0.5% formic acid, to provide the title compound as a colorless solid (0.057 g, 40% yield): ¹H-NMR (300 MHz; DMSO-d₆) δ 9.39-9.36 (m, 1H), 8.14 (d, J=0.4 Hz, 1H), 8.11 (d, J=5.0 Hz, 1H), 7.84 (d, J=0.5 Hz, 1H), 7.41-7.29 (m, 5H), 6.66 (d, J=5.0 Hz, 1H), 5.43-5.24 (m, 1H), 4.48 (quintet, J=6.6 Hz, 1H), 3.78-3.59 (m, 4H), 2.16-2.07 (m, 2H), 1.39 (d, J=6.7 Hz, 6H); MS (ES+) m/z 394.2 (M+1).

### Example 188

### Synthesis of (S)-N-(4-(2,5-difluorophenyl)-2-(3-fluoropyrrolidin-1-yl)pyridin-3-yl)-6-isopropylnicotinamide

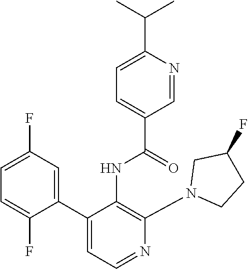 [SMILES: CC(C)c1ccc(C(=O)Nc2c(-c3cc(F)ccc3F)ccnc2N2CC[C@H](F)C2)cn1]

### Step 1. Preparation of 2-chloro-4-(2,5-difluorophenyl)-3-nitropyridine

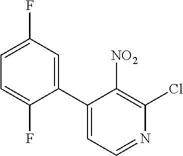 [SMILES: O=[N+]([O-])c1c(-c2cc(F)ccc2F)ccnc1Cl]

A mixture of 2,4-dichloro-3-nitropyridine (2.5 g, 13 mmol), 1,4-dioxane (25 mL), and water (8.6 mL) was sparged with nitrogen for 10 min. To the mixture was added 2,5-difluorophenylboronic acid (2.0 g, 13 mmol), potassium carbonate (2.7 g, 19 mmol), and [1,1′-bis(diphenylphosphino)ferrocene]dichloropalladium(II), complex with dichloromethane (1.1 g, 1.3 mmol). The reaction mixture was sparged with nitrogen for 2 minutes then stirred at 60° C. for 3 h. After cooling to ambient temperature, the reaction mixture was diluted with ethyl acetate (300 mL). The organic layer was washed with saturated ammonium chloride (2×100 mL). The organic solution was dried over anhydrous magnesium sulfate, filtered, and concentrated in vacuo. Purification of the residue by column chromatography, eluting with 5-35% ethyl acetate in heptane, afforded the title compound as a colorless oil that solidified upon standing (1.74 g, 50% yield).

### Step 2. Preparation of (S)-4-(2,5-difluorophenyl)-2-(3-fluoropyrrolidin-1-yl)-3-nitropyridine

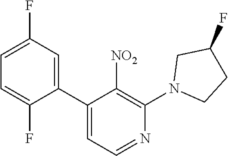 [SMILES: O=[N+]([O-])c1c(-c2cc(F)ccc2F)ccnc1N1CC[C@H](F)C1]

To a mixture of 2-chloro-4-(2,5-difluorophenyl)-3-nitropyridine (0.85 g, 3.2 mmol), anhydrous potassium carbonate (1.35 g, 9.60 mmol), and (S)-3-fluoropyrrolidine hydrochloride (0.44 g, 3.5 mmol) was added N,N-dimethylformamide (11 mL). The flask was sealed and heated to 70° C. for 72 h. The reaction mixture was diluted with ethyl acetate (150 mL). The organic layer was washed with saturated ammonium chloride (2×50 mL). The organic solution was dried over anhydrous magnesium sulfate, filtered, and concentrated in vacuo. Purification of the residue by column chromatography, eluting with 5-35% ethyl acetate in heptane, afforded the title compound as a yellow oil (0.77 g, 74% yield): MS (ES+) m/z 324.2 (M+1).

### Step 3. Preparation of (S)-4-(2,5-difluorophenyl)-2-(3-fluoropyrrolidin-1-yl)pyridin-3-amine

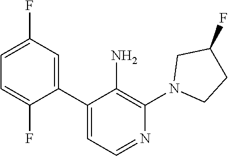 [SMILES: Nc1c(-c2cc(F)ccc2F)ccnc1N1CC[C@H](F)C1]

To a mixture of (S)-4-(2,5-difluorophenyl)-2-(3-fluoropyrrolidin-1-yl)-3-nitropyridine (0.77 g, 0.24 mmol) was added with anhydrous methanol (20 mL), and 10% palladium on carbon (0.23 g,). The reaction vessel was sealed and the reaction mixture was sparged with hydrogen gas for 5 min. The reaction was stirred under a hydrogen atmosphere for 24 h. The reaction mixture was filtered through diatomaceous earth (i.e., Celite®), washed with methanol (5×20 mL) and concentrated in vacuo. The resulting brown oil was used as is (0.6 g, 86% yield): MS (ES+) m/z 294.2 (M+1).

### Step 4. Preparation of (S)-N-(4-(2,5-difluorophenyl)-2-(3-fluoropyrrolidin-1-yl)pyridin-3-yl)-6-isopropylnicotinamide

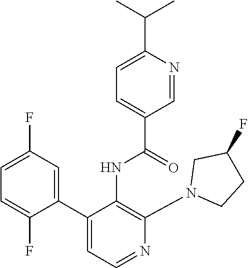 [SMILES: CC(C)c1ccc(C(=O)Nc2c(-c3cc(F)ccc3F)ccnc2N2CC[C@H](F)C2)cn1]

To a mixture of (S)-2-(3-fluoropyrrolidin-1-yl)-4-phenylpyridin-3-amine (0.093 g, 0.36 mmol), 1-isopropyl-1H-pyrazole-4-carboxylic acid (0.11 g, 0.73 mmol) and 2-chloro-1-methylpyridinium iodide (0.37 g, 1.5 mmol) was added anhydrous tetrahydrofuran (4.5 mL). The solution was heated at 65° C. for 5 min before N-ethyl-N-isopropylpropan-2-amine (0.47 g, 3.6 mmol) was added. The reaction mixture was stirred at 65° C. for 14 h. The reaction mixture was cooled to ambient temperature and diluted with ethyl acetate (200 mL). The reaction mixture was washed with saturated ammonium chloride solution (2×100 mL). The organic layer was dried over anhydrous magnesium sulfate, filtered and concentrated in vacuo. The residue was purified by preparative HPLC, eluting with a gradient of 10 to 90% of acetonitrile in water containing 0.5% formic acid, to provide the title compound as a colorless solid (0.057 g, 40% yield): ¹H-NMR (300 MHz; DMSO-d₆) δ 10.03 (d, J=0.4 Hz, 1H), 8.75 (dd, J=2.3, 0.7 Hz, 1H), 8.17 (d, J=4.9 Hz, 1H), 7.95 (dd, J=8.2, 2.4 Hz, 1H), 7.37 (dd, J=8.1, 0.4 Hz, 1H), 7.34-7.11 (m, 3H), 6.72 (dd, J=5.0, 0.7 Hz, 1H), 5.44-5.26 (m, 1H), 3.84-3.61 (m, 4H), 3.05 (quintet, J=6.9 Hz, 1H), 2.18-1.94 (m, 2H), 1.23 (dd, J=6.3, 4.2 Hz, 6H); MS (ES+) m/z: 441.2 (M+1).

### Example 189

### Synthesis of N-(2-(3,3-difluoropyrrolidin-1-yl)-4-phenylpyridin-3-yl)-2-isopropylpyrimidine-5-carboxamide

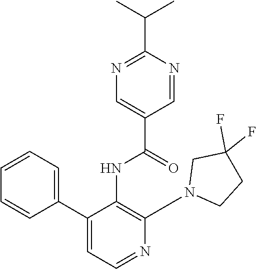 [SMILES: CC(C)c1ncc(C(=O)Nc2c(-c3ccccc3)ccnc2N2CCC(F)(F)C2)cn1]

### Step 1. Preparation of N-(2-(3,3-difluoropyrrolidin-1-yl)-4-phenylpyridin-3-yl)-2-isopropylpyrimidine-5-carboxamide

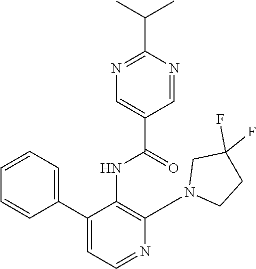 [SMILES: CC(C)c1ncc(C(=O)Nc2c(-c3ccccc3)ccnc2N2CCC(F)(F)C2)cn1]

To a mixture of 2-(3,3-difluoropyrrolidin-1-yl)-4-phenylpyridin-3-amine (0.075 g, 0.27 mmol), 2-isopropylpyrimidine-5-carboxylic acid (0.091 g, 0.54 mmol) and 2-chloro-1-methylpyridinium iodide (0.28 g, 1.1 mmol) was added anhydrous tetrahydrofuran (3.4 mL). The solution was heated at 65° C. for 5 min before N-ethyl-N-isopropylpropan-2-amine (0.35 g, 1.9 mmol) was added. The reaction mixture was stirred at 65° C. for 14 h. The reaction mixture was cooled to ambient temperature and diluted with ethyl acetate (150 mL). The reaction mixture was washed with saturated ammonium chloride solution (2×50 mL). The organic layer was dried over anhydrous magnesium sulfate, filtered and concentrated in vacuo. The residue was purified by preparative HPLC, eluting with a gradient of 5 to 45% of acetonitrile in water containing 0.1% trifluoroacetic acid, to provide the title compound as a colorless solid (0.077 g, 66% yield): ¹H-NMR (300 MHz; DMSO-d₆) δ 10.17 (s, 1H), 8.92 (d, J=3.1 Hz, 2H), 8.20 (d, J=5.0 Hz, 1H), 7.39-7.30 (m, 5H), 6.80 (d, J=5.0 Hz, 1H), 3.96-3.68 (m, 4H), 3.17 (quintet, J=6.9 Hz, 1H), 2.48-2.36 (m, 2H), 1.28 (t, J=6.4 Hz, 6H); MS (ES+) m/z 424.2 (M+1).

### Example 190

### Synthesis of N-(4-(2,5-difluorophenyl)-2-(3,3-difluoropyrrolidin-1-yl)pyridin-3-yl)-2-isopropylpyrimidine-5-carboxamide

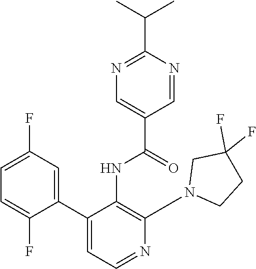 [SMILES: CC(C)c1ncc(C(=O)Nc2c(-c3cc(F)ccc3F)ccnc2N2CCC(F)(F)C2)cn1]

### Step 1. Preparation of 4-(2,5-difluorophenyl)-2-(3,3-difluoropyrrolidin-1-yl)-3-nitropyridine

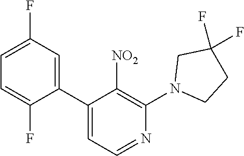 [SMILES: O=[N+]([O-])c1c(-c2cc(F)ccc2F)ccnc1N1CCC(F)(F)C1]

To a mixture of 2-chloro-4-(2,5-difluorophenyl)-3-nitropyridine (0.85 g, 3.2 mmol), anhydrous potassium carbonate (1.35 g, 9.60 mmol), and 3,3-difluoropyrrolidine hydrochloride (0.51 g, 3.5 mmol) was added N,N-dimethylformamide (11 mL). The flask was sealed and stirred at ambient temperature for 72 h. The reaction mixture was diluted with ethyl acetate (150 mL). The organic layer was washed with saturated ammonium chloride (2×50 mL). The organic solution was dried over anhydrous magnesium sulfate, filtered, and concentrated in vacuo. Purification of the residue by column chromatography, eluting with 5-35% ethyl acetate in heptane, afforded the title compound as a yellow oil (0.62 g, 56% yield).

### Step 2. Preparation of 4-(2,5-difluorophenyl)-2-(3,3-difluoropyrrolidin-1-yl)pyridin-3-amine

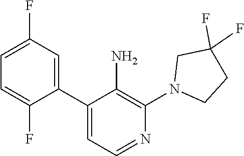 [SMILES: Nc1c(-c2cc(F)ccc2F)ccnc1N1CCC(F)(F)C1]

To 4-(2,5-difluorophenyl)-2-(3,3-difluoropyrrolidin-1-yl)-3-nitropyridine (0.62 g, 1.8 mmol) was added anhydrous methanol (7.3 mL) and 10% palladium on carbon (0.19 g). The reaction vessel was sealed and the reaction mixture was sparged with hydrogen gas for 5 min. The reaction was stirred under a hydrogen atmosphere for 24 h. The reaction mixture was filtered through diatomaceous earth (i.e., Celite®), washed with methanol (5×20 mL) and concentrated in vacuo. The resulting red oil was used as is (0.5 g, 88% yield): MS (ES+) m/z: 312.2 (M+1).

### Step 3. Preparation of N-(4-(2,5-difluorophenyl)-2-(3,3-difluoropyrrolidin-1-yl)pyridin-3-yl)-2-isopropylpyrimidine-5-carboxamide

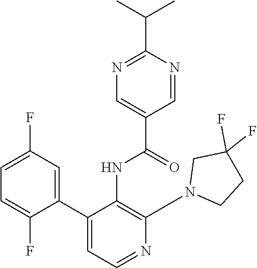 [SMILES: CC(C)c1ncc(C(=O)Nc2c(-c3cc(F)ccc3F)ccnc2N2CCC(F)(F)C2)cn1]

To a mixture of 4-(2,5-difluorophenyl)-2-(3,3-difluoropyrrolidin-1-yl)pyridin-3-amine (0.10 g, 0.32 mmol), 2-isopropylpyrimidine-5-carboxylic acid (0.080 g, 0.48 mmol) and 2-chloro-1-methylpyridinium iodide (0.25 g, 0.96 mmol) was added with anhydrous tetrahydrofuran (4.0 mL). The solution was heated at 65° C. for 5 min before N-ethyl-N-isopropylpropan-2-amine (0.42 g, 3.2 mmol) was added. The reaction mixture was stirred at 65° C. for 14 h. The reaction mixture was cooled to ambient temperature and diluted with ethyl acetate (150 mL). The reaction mixture was washed with saturated ammonium chloride solution (2×50 mL). The organic layer was dried over anhydrous magnesium sulfate, filtered and concentrated in vacuo. Purification of the residue by column chromatography, eluting with 15-100% ethyl acetate in heptane, provide the title compound as a colorless solid (0.065 g, 44% yield): ¹H-NMR (300 MHz; DMSO-d₆) δ 10.23 (s, 1H), 8.92 (s, 2H), 8.23 (d, J=4.9 Hz, 1H), 7.33 (td, J=9.1, 4.5 Hz, 1H), 7.24 (td, J=8.1, 4.3 Hz, 1H), 7.17-7.14 (m, 1H), 6.85 (d, J=4.9 Hz, 1H), 3.93-3.87 (m, 2H), 3.76-3.73 (m, 2H), 3.18 (dt, J=13.8, 6.9 Hz, 1H), 2.50-2.40 (m, 3H), 1.27 (t, J=5.8 Hz, 6H); MS (ES+) m/z 460.2(M+1).

### Example 191

### Synthesis of 6-(3,3-difluoropyrrolidin-1-yl)-12-isopropyl-5,8,12,13-tetraazatetracyclo[15.4.0.02,7.010,14]henicosa-1(21),2(7),3,5,10,13,17,19-octaen-9-one

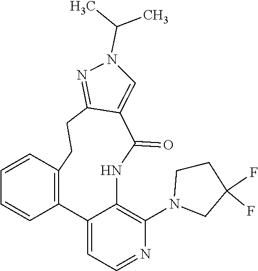 [SMILES: CC(C)n1cc2c(n1)CCc1ccccc1-c1ccnc(N3CCC(F)(F)C3)c1NC2=O]

### Step 1. Preparation of ethyl 3-[(E)-2-[2-[3-amino-2-(3,3-difluoropyrrolidin-1-yl)-4-pyridyl]phenyl]vinyl]-1-isopropyl-pyrazole-4-carboxylate

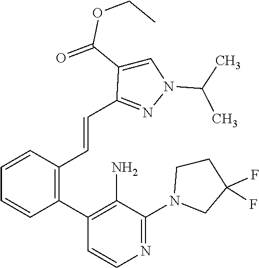 [SMILES: CCOC(=O)c1cn(C(C)C)nc1/C=C/c1ccccc1-c1ccnc(N2CCC(F)(F)C2)c1N]

To a solution of 2-(3,3-difluoropyrrolidin-1-yl)-4-(2-vinylphenyl)pyridin-3-amine hydrochloride (1.00 g, 2.96 mmol), ethyl 3-bromo-1-isopropyl-pyrazole-4-carboxylate (1.63 g, 5.92 mmol), tris-o-tolylphosphane (0.541 g, 1.78 mmol) and triethylamine (1.65 mL, 0.251 mmol) in N,N-dimethylformamide (20.0 mL) was added palladium(II) acetate (0.199 g, 0.888 mmol), and the mixture was stirred at 120° C. for 4 days. After cooling to ambient temperature, the mixture was diluted with saturated aqueous sodium bicarbonate solution (150 mL). The aqueous phase was extracted with ethyl acetate (3×150 mL). The organic layer was washed with brine (150 mL), dried over anhydrous sodium sulfate, filtered, and concentrated in vacuo. Purification of the residue by column chromatography, eluting with a gradient of 0-100% of ethyl acetate in hexanes, afforded the title compound as a brown solid (1.15 g, 81% yield): ¹H NMR (400 MHz; DMSO-d₆) δ 8.27 (s, 1H), 7.83 (d, J=6.7 Hz, 1H), 7.65 (d, J=4.9 Hz, 1H), 7.56 (d, J=16.4 Hz, 1H), 7.49 (td, J=7.4, 1.1 Hz, 1H), 7.43 (td, J=7.4, 1.3 Hz, 1H), 7.26 (dd, J=7.5, 1.3 Hz, 1H), 7.21 (d, J=16.4 Hz, 1H), 6.69 (d, J=4.9 Hz, 1H), 4.51-4.39 (m, 1H), 4.26 (s, 2H), 4.22 (q, J=7.1 Hz, 2H), 3.90 (dt, J=26.9, 13.4 Hz, 1H), 3.67 (dt, J=10.3, 7.4 Hz, 1H), 3.59-3.47 (m, 1H), 3.39-3.32 (m, 1H), 2.49-2.35 (m, 2H), 1.39-1.33 (m, 6H), 1.28 (t, J=7.1 Hz, 3H); MS (ES+) m/z 481.9 (M+1).

### Step 2. Preparation of ethyl 3-[2-[2-[3-amino-2-(3,3-difluoropyrrolidin-1-yl)-4-pyridyl]phenyl]ethyl]-1-isopropyl-pyrazole-4-carboxylate

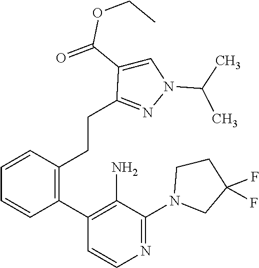 [SMILES: CCOC(=O)c1cn(C(C)C)nc1CCc1ccccc1-c1ccnc(N2CCC(F)(F)C2)c1N]

To a mixture of palladium (10% on carbon matrix, 1.05 g, 0.986 mmol) in methanol (25.0 mL) was added ethyl 3-[(E)-2-[2-[3-amino-2-(3,3-difluoropyrrolidin-1-yl)-4-pyridyl]phenyl]vinyl]-1-isopropyl-pyrazole-4-carboxylate (0.950 g, 1.97 mmol), and the mixture was stirred at 22° C. for 2 h under hydrogen. The mixture was diluted with dichloromethane (100 mL) and filtered through a bed of diatomaceous earth (i.e., Celite®). The solid was washed with dichloromethane (300 mL). Concentration of the filtrate in vacuo afforded the title compound as a brown oil (0.960 g, 95% yield): ¹H NMR (400 MHz; CDCl₃) δ 7.74 (d, J=5.1 Hz, 1H), 7.73 (s, 1H), 7.25-7.18 (m, 3H), 7.13-7.09 (m, 1H), 6.69 (d, J=4.9 Hz, 1H), 4.34-4.23 (m, 1H), 4.21-4.13 (m, 2H), 3.73-3.61 (m, 4H), 3.61-3.44 (m, 2H), 3.05 (dt, J=12.6, 6.9 Hz, 1H), 2.97-2.83 (m, 2H), 2.83-2.72 (m, 1H), 2.39 (tt, J=14.4, 7.1 Hz, 2H), 1.38-1.33 (m, 6H), 1.26 (t, J=7.1 Hz, 3H); MS (ES+) m/z 484.3 (M+1).

### Step 3. Preparation of lithium 3-[2-[2-[3-amino-2-(3,3-difluoropyrrolidin-1-yl)-4-pyridyl]phenyl]ethyl]-1-isopropyl-pyrazole-4-carboxylate

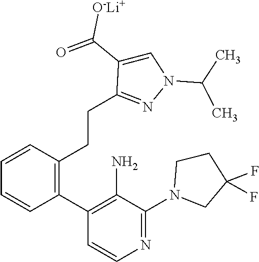

To a solution of ethyl 3-[2-[2-[3-amino-2-(3,3-difluoropyrrolidin-1-yl)-4-pyridyl]phenyl]ethyl]-1-isopropyl-pyrazole-4-carboxylate (0.950 g, 1.96 mmol) in 1,4-dioxane (15.0 mL) and water (15.0 mL) was added lithium hydroxide monohydrate (0.412 g, 9.82 mmol) and the mixture was stirred at 90° C. for 2 h. After cooling to ambient temperature, the mixture was concentrated in vacuo. Purification of the residue by reverse phase chromatography, eluting with a gradient of 5-100% of acetonitrile in water containing 10 mM of ammonium bicarbonate, afforded the title compound as colorless solid (0.760 g, 84% yield): ¹H NMR (400 MHz; DMSO-d₆) δ 7.66 (s, 1H), 7.62 (d, J=4.9 Hz, 1H), 7.40-7.34 (m, 1H), 7.26 (qd, J=7.3, 3.7 Hz, 2H), 7.08 (dd, J=7.3, 1.6 Hz, 1H), 6.66 (d, J=4.9 Hz, 1H), 4.24 (hept, J=13.4, 6.7 Hz, 1H), 4.17 (s, 2H), 3.71 (td, J=13.8, 3.7 Hz, 2H), 3.48 (ddd, J=17.3, 8.6, 5.7 Hz, 2H), 3.07-2.93 (m, 1H), 2.93-2.63 (m, 3H), 2.42 (tt, J=14.5, 7.1 Hz, 2H), 1.27 (d, J=6.7 Hz, 6H); MS (ES+) m/z 456.5 (M+1).

### Step 4. Preparation of 6-(3,3-difluoropyrrolidin-1-yl)-12-isopropyl-5,8,12,13-tetrazatetracyclo[15.4.0.02,7.010,14]henicosa-1(21),2(7),3,5,10,13,17,19-octaen-9-one

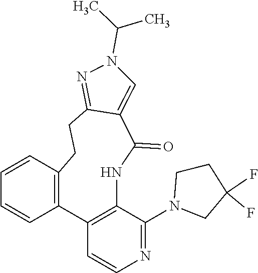 [SMILES: CC(C)n1cc2c(n1)CCc1ccccc1-c1ccnc(N3CCC(F)(F)C3)c1NC2=O]

To a mixture of 2-chloro-1-methyl-pyridin-1-ium iodide (0.221 g, 0.867 mmol) in dichloromethane (31.5 mL) was added lithium 3-[2-[2-[3-amino-2-(3,3-difluoropyrrolidin-1-yl)-4-pyridyl]phenyl]ethyl]-1-isopropyl-pyrazole-4-carboxylate (0.100 g, 0.217 mmol) and triethylamine (0.151 mL, 1.08 mmol) in dichloromethane (10.5 mL) over 4 h at 30° C., and the mixture was stirred at 30° C. for 18 h. After cooling to ambient temperature, the mixture was diluted with saturated aqueous sodium bicarbonate (100 mL), and the aqueous phase was extracted with ethyl acetate (3×100 mL). The organic phase was washed with brine (300 ml), dried over anhydrous sodium sulfate, filtered and concentrated in vacuo. Purification by column chromatography, eluting with a gradient of 0-10% of methanol in dichloromethane, followed by preparative reverse phase HPLC, eluting with a gradient of 36-46% of acetonitrile in water, containing 10 mM of ammonium formate, afforded the title compound as a colorless solid (0.0570 g, 60%): ¹H NMR (400 MHz; DMSO-d₆) δ 8.96 (s, 1H), 8.25-7.84 (m, 1H), 7.83-7.39 (m, 1H), 7.38-7.21 (m, 1H), 7.22-7.05 (m, 2H), 7.05-6.89 (m, 1H), 6.89-6.37 (m, 1H), 4.42-4.00 (m, 2H), 4.00-3.33 (m, 4H), 3.16-2.51 (m, 3H), 2.49-1.82 (m, 2H), 1.45-1.09 (m, 6H); MS (ES+) m/z 438.3 (M+1).

### Example 192

### Synthesis of N-[2-(3,3-difluoropyrrolidin-1-yl)-4-(2-fluoro-5-methoxy-phenyl)-3-pyridyl]-2-(dimethylamino)pyrimidine-5-carboxamide

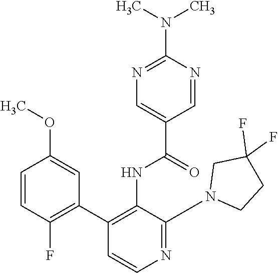 [SMILES: COc1ccc(F)c(-c2ccnc(N3CCC(F)(F)C3)c2NC(=O)c2cnc(N(C)C)nc2)c1]

To a solution of 2-(3,3-difluoropyrrolidin-1-yl)-4-(2-fluoro-5-methoxy-phenyl)pyridin-3-amine (0.0500 g, 0.139 mmol), 2-(dimethylamino)pyrimidine-5-carboxylic acid (0.0349 g, 0.209 mmol), and 2-chloro-1-methyl-pyridin-1-ium iodide (0.142 g, 0.557 mmol) in tetrahydrofuran (1.00 mL) was added N,N-diisopropylethylamine (0.0953 mL, 0.557 mmol), and the mixture was stirred at 65° C. for 18 h. 2-(Dimethylamino)pyrimidine-5-carboxylic acid (0.0349 g, 0.209 mmol) was added and the mixture was stirred at 65° C. for 4 h. After cooling to ambient temperature, the mixture was diluted with saturated aqueous sodium bicarbonate (15 mL), and the aqueous phase was extracted with ethyl acetate (3×15 mL). The organic phase was dried over anhydrous sodium sulfate, filtered, and concentrated in vacuo. Purification of the residue by column chromatography, eluting with a gradient of 0-15% of methanol in dichloromethane, followed by preparative HPLC, eluting with a gradient of 40-50% of acetonitrile in water containing 10 mM of ammonium bicarbonate, afforded the title compound as a colorless solid (0.0260 g, 41% yield): ¹H NMR (400 MHz; DMSO-d₆) δ 9.66 (s, 1H), 8.62 (s, 2H), 8.17 (d, J=5.0 Hz, 1H), 7.15 (t, J=9.2 Hz, 1H), 6.88 (dt, J=8.9, 3.6 Hz, 1H), 6.82 (dd, J=5.8, 3.1 Hz, 1H), 6.79 (dd, J=5.1, 0.5 Hz, 1H), 3.98-3.67 (m, 4H), 3.65 (s, 3H), 3.15 (s, 6H), 2.43 (ddd, J=21.6, 14.4, 7.3 Hz, 2H); MS (ES+) m/z 473.3 (M+1).

### Example 193

### Synthesis of N-[2-(3,3-difluoropyrrolidin-1-yl)-4-(2-fluoro-4-hydroxy-phenyl)-3-pyridyl]-2-isopropyl-pyrimidine-5-carboxamide

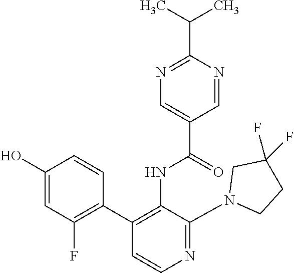 [SMILES: CC(C)c1ncc(C(=O)Nc2c(-c3ccc(O)cc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0750 g, 0.158 mmol), (2-fluoro-4-hydroxy-phenyl)boronic acid (0.0494 g, 0.317 mmol) and [1,1′ bis(diphenylphosphino)ferrocene]dichloropalladium(II), complex with dichloromethane (0.0259 g, 0.0317 mmol) in 1,4-dioxane (1.50 mL) and water (0.300 mL) was added potassium carbonate (0.0548 g, 0.396 mmol), and the mixture was stirred at 100° C. for 1 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium bicarbonate, followed by preparative reverse phase HPLC, eluting with a gradient of 40-50% of acetonitrile in water containing 10 mM of ammonium bicarbonate, afforded the title compound as a colorless solid (0.0390 g, 53% yield): ¹H NMR (400 MHz; DMSO-d₆) δ 10.10 (s, 1H), 9.98 (s, 1H), 8.92 (s, 2H), 8.16 (d, J=5.0 Hz, 1H), 7.10 (t, J=8.4 Hz, 1H), 6.81-6.71 (m, 1H), 6.64-6.51 (m, 2H), 3.88 (bs, 2H), 3.73 (bs, 2H), 3.18 (dt, J=13.8, 6.9 Hz, 1H), 2.42 (tt, J=14.2, 7.3 Hz, 2H), 1.28 (d, J=6.9 Hz, 6H); ¹⁹F NMR (376 MHz, DMSO-d₆) δ-101.01 (s), −112.72-114.17 (m); MS (ES+) m/z 458.2 (M+1).

### Example 194

### Synthesis of 6-(3,3-difluoropyrrolidin-1-yl)-13-isopropyl-5,8,12,13-tetrazatetracyclo[15.4.0.02,7.010,14]henicosa-1(21),2(7),3,5,10(14),11,17,19-octaen-9-one

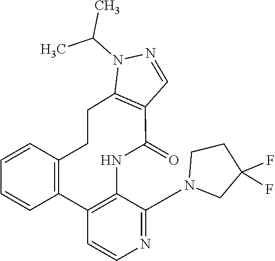 [SMILES: CC(C)n1ncc2c1CCc1ccccc1-c1ccnc(N3CCC(F)(F)C3)c1NC2=O]

### Step 1. Preparation of ethyl 5-bromo-1-isopropyl-pyrazole-4-carboxylate

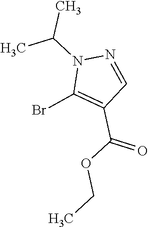

To a solution of ethyl 3-bromo-1H-pyrazole-4-carboxylate (3.00 g, 13.0 mmol) and caesium carbonate (8.48 g, 26.0 mmol) in acetonitrile (72.0 mL) was added 2-bromopropane (1.83 mL, 19.5 mmol), and the mixture was stirred at 60° C. for 1 h. After cooling to ambient temperature, the mixture was concentrated in vacuo, and the residue was diluted with ethyl acetate (200 mL) and saturated aqueous sodium bicarbonate (200 mL). The aqueous phase was extracted with ethyl acetate (2×200 mL). The organic phase was washed with brine, dried over anhydrous sodium sulfate, filtered, and concentrated in vacuo. Purification of the residue by column chromatography, eluting with a gradient of 0-12% of ethyl acetate in hexanes, afforded the title compound as a colorless oil (0.89 g, 26% yield): ¹H NMR (400 MHz; CDCl₃) δ 7.96 (d, J=0.5 Hz, 1H), 4.81-4.66 (m, 1H), 4.29 (q, J=7.1 Hz, 2H), 1.46 (d, J=6.6 Hz, 6H), 1.33 (t, J=7.1 Hz, 3H); MS (ES+) m/z 260.0 (M+1), 262.0 (M+1).

### Step 2. Preparation of ethyl ethyl 5-[(E)-2-[2-[3-amino-2-(3,3-difluoropyrrolidin-1-yl)-4-pyridyl]phenyl]vinyl]-1-isopropyl-pyrazole-4-carboxylate

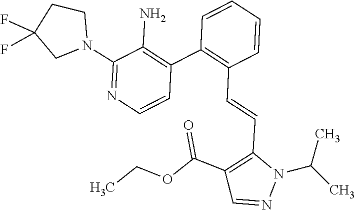 [SMILES: CCOC(=O)c1cnn(C(C)C)c1/C=C/c1ccccc1-c1ccnc(N2CCC(F)(F)C2)c1N]

To a solution of 2-(3,3-difluoropyrrolidin-1-yl)-4-(2-vinylphenyl)pyridin-3-amine hydrochloride (0.400 g, 1.18 mmol), ethyl 5-bromo-1-isopropyl-pyrazole-4-carboxylate (0.651 g, 2.37 mmol), tris-o-tolylphosphane (0.216 g, 0.710 mmol) and triethylamine (0.661 mL, 4.74 mmol) in N,N-dimethylformamide (8.00 mL) was added palladium(II) acetate (0.0798 g, 0.355 mmol), and the mixture was stirred at 120° C. for 3 days. After cooling to ambient temperature, the mixture was diluted with saturated aqueous sodium bicarbonate (100 mL). The aqueous phase was extracted with ethyl acetate (3×100 mL). The organic layer was washed with brine (100 mL), dried over anhydrous sodium sulfate, filtered, and concentrated in vacuo. Purification of the residue by column chromatography, eluting with a gradient of 0-30% of ethyl acetate in hexanes, afforded the title compound as a yellow solid (0.431 g, 76% yield): ¹H NMR (400 MHz; CDCl₃) δ 7.90 (dd, J=7.7, 1.5 Hz, 1H), 7.87 (s, 1H), 7.82 (d, J=4.9 Hz, 1H), 7.51-7.41 (m, 2H), 7.35 (d, J=16.8 Hz, 1H), 7.30-7.27 (m, 1H), 6.80 (d, J=16.9 Hz, 1H), 6.76 (d, J=4.9 Hz, 1H), 4.44 (hept, J=6.6 Hz, 1H), 4.26 (q, J=7.1 Hz, 2H), 3.80-3.62 (m, 2H), 3.61 (br s, 2H), 3.59-3.44 (m, 2H), 2.42 (dq, J=21.4, 7.1 Hz, 2H), 1.33 (d, J=14.2 Hz, 3H), 1.33 (d, J=6.6 Hz, 3H), 1.28 (d, J=6.6 Hz, 3H); MS (ES+) m/z 481.9 (M+1).

### Step 3. Preparation of ethyl 5-[2-[2-[3-amino-2-(3,3-difluoropyrrolidin-1-yl)-4-pyridyl]phenyl]ethyl]-1-isopropyl-pyrazole-4-carboxylate

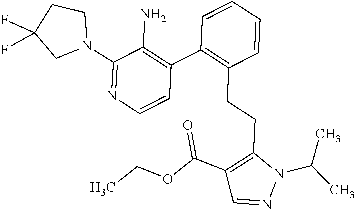 [SMILES: CCOC(=O)c1cnn(C(C)C)c1CCc1ccccc1-c1ccnc(N2CCC(F)(F)C2)c1N]

To a mixture of palladium (10% on carbon matrix, 0.476 g, 0.448 mmol) in methanol (17.2 mL) was added ethyl 5-[(E)-2-[2-[3-amino-2-(3,3-difluoropyrrolidin-1-yl)-4-pyridyl]phenyl]vinyl]-1-isopropyl-pyrazole-4-carboxylate (0.431 g, 0.895 mmol) under hydrogen and the mixture was stirred at 22° C. for 1 h 15 minutes. The mixture was diluted with dichloromethane (100 mL) and filtered through a bed of diatomaceous earth (i.e., Celite®). The solid was washed with dichloromethane (300 mL). Concentration of the filtrate in vacuo afforded the title compound as a brown solid (0.393 g, 91% yield): MS (ES+) m/z 484.3 (M+1).

### Step 4. Preparation of lithium 5-[2-[2-[3-amino-2-(3,3-difluoropyrrolidin-1-yl)-4-pyridyl]phenyl]ethyl]-1-isopropyl-pyrazole-4-carboxylate

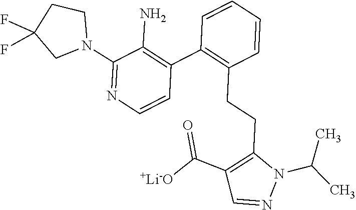

To a solution of ethyl 5-[2-[2-[3-amino-2-(3,3-difluoropyrrolidin-1-yl)-4-pyridyl]phenyl]ethyl]-1-isopropyl-pyrazole-4-carboxylate (0.393 g, 0.813 mmol) in 1,4-dioxane (6.21 mL) and water (6.21 mL) was added lithium hydroxide monohydrade (0.171 g, 4.06 mmol) and the mixture was stirred at 90° C. for 2 h. After cooling to ambient temperature, the mixture was concentrated in vacuo. Purification of the residue by reverse phase chromatography, eluting with a gradient of 5-50% of acetonitrile in water, containing 10 mM of ammonium bicarbonate, afforded the title compound as a colorless solid (0.370 mg, 94%): ¹H NMR (300 MHz; DMSO-d₆) δ 7.66 (s, 1H), 7.62 (d, J=4.9 Hz, 1H), 7.40-7.34 (m, 1H), 7.26 (qd, J=7.3, 3.7 Hz, 2H), 7.08 (dd, J=7.3, 1.6 Hz, 1H), 6.66 (d, J=4.9 Hz, 1H), 4.24 (hept, J=13.4, 6.7 Hz, 1H), 4.17 (s, 2H), 3.71 (td, J=13.8, 3.7 Hz, 2H), 3.48 (ddd, J=17.3, 8.6, 5.7 Hz, 2H), 3.07-2.93 (m, 1H), 2.93-2.63 (m, 3H), 2.42 (tt, J=14.5, 7.1 Hz, 2H), 1.27 (d, J=6.7 Hz, 6H); MS (ES+) m/z 456.5 (M+1).

### Step 5. Preparation of 6-(3,3-difluoropyrrolidin-1-yl)-13-isopropyl-5,8,12,13-tetrazatetracyclo[15.4.0.02,7.010,14]henicosa-1(21),2(7),3,5,10(14),11,17,19-octaen-9-one

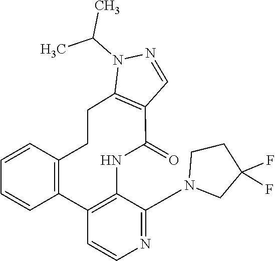 [SMILES: CC(C)n1ncc2c1CCc1ccccc1-c1ccnc(N3CCC(F)(F)C3)c1NC2=O]

To a mixture of 2-chloro-1-methyl-pyridin-1-ium iodide (0.221 g, 0.867 mmol) in dichloromethane (31.5 mL) was added lithium;5-[2-[2-[3-amino-2-(3,3-difluoropyrrolidin-1-yl)-4-pyridyl]phenyl]ethyl]-1-isopropyl-pyrazole-4-carboxylate (0.100 g, 0.217 mmol) and triethylamine (0.151 mL, 1.08 mmol) in dichloromethane (10.5 mL) over 4 h at 30° C., and the mixture was stirred at 30° C. for 18 h. After cooling to ambient temperature, the mixture was diluted with saturated aqueous sodium bicarbonate (100 mL), and the aqueous phase was extracted with ethyl acetate (3×100 mL). The organic phase was washed with brine (300 ml), dried over anhydrous sodium sulfate, filtered, and concentrated in vacuo. Purification by column chromatography, eluting with a gradient of 0-10% of methanol in dichloromethane, followed by preparative reverse phase HPLC, eluting with a gradient of 36-46% of acetonitrile in water, containing 10 mM of ammonium formate, afforded the title compound as a colorless solid (0.0480 g, 51%): ¹H NMR (400 MHz; DMSO-d₆) δ 9.25-8.87 (m, 1H), 8.19-7.62 (m, 1H), 7.42-7.03 (m, 4H), 7.03-6.91 (m, 1H), 6.81-6.28 (m, 1H), 4.62 (s, 1H), 4.21-3.96 (m, 1H), 3.94-3.74 (m, 1H), 3.74-3.38 (m, 3H), 3.26-2.72 (m, 3H), 2.43-2.05 (m, 2H), 1.50-0.98 (m, 6H); MS (ES+) m/z 438.3 (M+1).

### Example 195

### Synthesis of N-[4-[4-(difluoromethyl)-2-fluoro-phenyl]-2-(3,3-difluoropyrrolidin-1-yl)-3-pyridyl]-2-isopropyl-pyrimidine-5-carboxamide

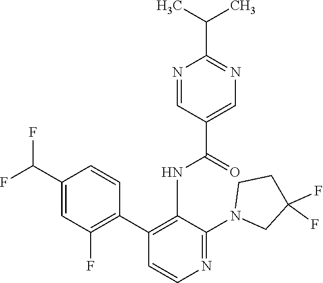 [SMILES: CC(C)c1ncc(C(=O)Nc2c(-c3ccc(C(F)F)cc3F)ccnc2N2CCC(F)(F)C2)cn1]

### Step 1. Preparation of N-[2-(3,3-difluoropyrrolidin-1-yl)-4-(2-fluoro-4-formyl-phenyl)-3-pyridyl]-2-isopropyl-pyrimidine-5-carboxamide

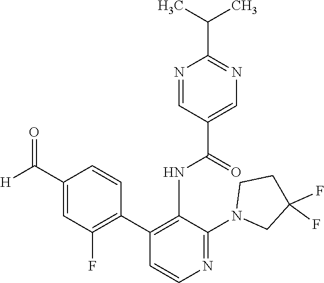 [SMILES: CC(C)c1ncc(C(=O)Nc2c(-c3ccc(C=O)cc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (95% pure, 0.150 g, 0.301 mmol), (2-fluoro-4-formyl-phenyl)boronic acid (0.101 g, 0.603 mmol), and potassium carbonate (0.104 g, 0.753 mmol) in 1,4-dioxane (2.40 mL) and water (0.800 mL) was added [1,1′-bis(diphenylphosphino)ferrocene]dichloropalladium(II), complex with dichloromethane (0.0738 g, 0.0904 mmol), and the mixture was stirred at 100° C. for 20 h. After cooling to ambient temperature, the mixture was diluted with ethyl acetate (30 mL). The mixture was passed through a bed of diatomaceous earth (i.e., Celite®). The solid was washed with ethyl acetate (150 mL), and the filtrate was concentrated in vacuo.

Purification of the residue by column chromatography, eluting with a gradient of 0-5% methanol in dichloromethane, afforded the title compound as a red solid (0.159 g, 96% yield): ¹H NMR (300 MHz; DMSO-d₆) δ 10.28 (s, 1H), 9.95 (d, J=1.6 Hz, 1H), 8.90 (s, 2H), 8.24 (d, J=5.0 Hz, 1H), 7.81-7.72 (m, 2H), 7.54 (t, J=7.4 Hz, 1H), 6.89-6.81 (m, 1H), 3.90 (t, 2H), 3.76 (t, J=7.4 Hz, 2H), 3.27-3.06 (m, 1H), 2.50-2.34 (m, 2H), 1.25 (d, J=6.9 Hz, 6H); MS (ES+) m/z 470.3 (M+1).

### Step 2. Preparation of N-[4-[4-(difluoromethyl)-2-fluoro-phenyl]-2-(3,3-difluoropyrrolidin-1-yl)-3-pyridyl]-2-isopropyl-pyrimidine-5-carboxamide

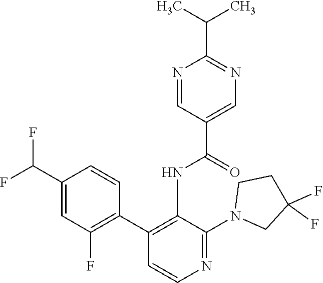 [SMILES: CC(C)c1ncc(C(=O)Nc2c(-c3ccc(C(F)F)cc3F)ccnc2N2CCC(F)(F)C2)cn1]

To a solution of N-[2-(3,3-difluoropyrrolidin-1-yl)-4-(2-fluoro-4-formyl-phenyl)-3-pyridyl]-2-isopropyl-pyrimidine-5-carboxamide (95% pure, 0.110 g, 0.223 mmol) in dichloromethane (3.30 mL) was added diethylaminosulfur trifluoride (0.0735 mL, 0.556 mmol), and the mixture was stirred at 22° C. for 18 h. The mixture was diluted with aqueous sodium hydroxide (2 M, 1.11 mL, 2.23 mmol) and stirred at 50° C. for 1 h. After cooling to ambient temperature, the mixture was diluted with saturated aqueous sodium bicarbonate (20 mL) and extracted with ethyl acetate (3×20 mL). The combined organic phase was washed with brine (20 mL), dried over anhydrous sodium sulfate, and concentrated in vacuo. Purification of the residue by column chromatography, eluting with a gradient of 0-10% of methanol in dichloromethane, followed by preparative HPLC, elution with a gradient of 52-62% of acetonitrile in water containing 10 mM of ammonium formate, afforded the title compound as a colorless solid (0.0110 g, 9% yield): ¹H NMR (300 MHz; DMSO-d₆) δ 10.24 (s, 1H), 8.89 (s, 2H), 8.23 (d, J=4.9 Hz, 1H), 7.59-7.35 (m, 3H), 7.02 (t, J=55.8 Hz, 1H), 6.83 (s, 1H), 4.04-3.66 (m, 4H), 3.16 (hept, J=6.2 Hz, 1H), 2.40 (dd, J=14.2, 7.1 Hz, 2H), 1.26 (d, J=6.9 Hz, 6H); ¹⁹F NMR (376 MHz; DMSO-d₆) δ-101.42, -110.50 (d, J=55.3 Hz), −113.08 (dd, J=9.4, 7.6 Hz); MS (ES+) m/z 492.2 (M+1).

### Example 196, 197, and 198

### Synthesis of 6-(3,3-difluoropyrrolidin-1-yl)-12-isopropyl-5,8,12,13-tetrazatetracyclo[16.4.0.02,7.010,14]docosa-1(22),2(7),3,5,10,13,18,20-octaen-9-one, (15R)-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9-one, and (15S)-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9 one

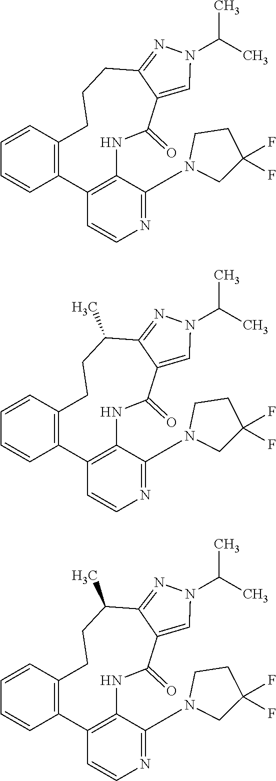 [SMILES: CC(C)n1cc2c(n1)CCCc1ccccc1-c1ccnc(N3CCC(F)(F)C3)c1NC2=O ; CC(C)n1cc2c(n1)[C@@H](C)CCc1ccccc1-c1ccnc(N3CCC(F)(F)C3)c1NC2=O ; CC(C)n1cc2c(n1)[C@H](C)CCc1ccccc1-c1ccnc(N3CCC(F)(F)C3)c1NC2=O]

### Step 1. Preparation of 4-(2-allylphenyl)-2-(3,3-difluoropyrrolidin-1-yl)pyridin-3-amine;hydrochloride

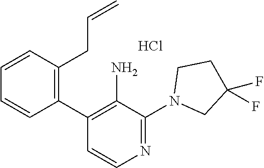 [SMILES: C=CCc1ccccc1-c1ccnc(N2CCC(F)(F)C2)c1N ; Cl]

To a solution of 2-(3,3-difluoropyrrolidin-1-yl)-4-iodo-pyridin-3-amine hydrochloride (0.700 g, 1.84 mmol), 2-(2-allylphenyl)-4,4,5,5-tetramethyl-1,3,2-dioxaborolane (0.748 g, 2.76 mmol), and potassium carbonate (0.890 g, 6.44 mmol) in 1,4-dioxane (22.0 mL) and water (7.33 mL) was added [1,1′-bis(diphenylphosphino)ferrocene]dichloropalladium(II), complex with dichloromethane (0.451 g, 0.552 mmol), and the mixture was stirred at 100° C. for 90 minutes. After cooling to ambient temperature, the mixture was diluted with saturated aqueous sodium bicarbonate solution (120 mL), and the aqueous phase was extracted with ethyl acetate (3×120 mL). The organic phase was washed with brine (120 mL), dried over anhydrous sodium sulfate, and concentrated in vacuo. The residue was purified by column chromatography, eluting with a gradient of 0-10% of methanol in dichloromethane. The residue was diluted with diethyl ether (5 mL) and hydrochloric acid (2 M in diethyl ether, 1.38 mL, 2.76 mmol) was added. Filtration of the solid, washing with diethyl ether (3×50 mL), afforded the title compound as a brown solid (0.35 g, 49% yield): ¹H NMR (300 MHz; DMSO-d₆) δ 7.62 (d, J=5.9 Hz, 1H), 7.50-7.35 (m, 3H), 7.17 (dd, J=7.7, 1.6 Hz, 1H), 6.88 (d, J=5.9 Hz, 1H), 5.80 (ddt, J=16.7, 10.1, 6.6 Hz, 1H), 5.00-4.83 (m, 2H), 4.19-3.97 (m, 2H), 3.95-3.75 (m, 2H), 3.20 (qd, J=15.5, 6.7 Hz, 2H), 2.57 (dt, J=14.8, 7.4 Hz, 2H); MS (ES+) m/z 316.2 (M+1).

### Step 2. Synthesis of ethyl 3-bromo-1-isopropyl-pyrazole-4-carboxylate

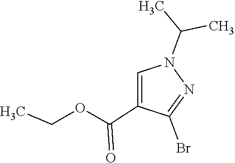

To a solution of ethyl 3-bromo-1H-pyrazole-4-carboxylate (3.00 g, 13.0 mmol) and cesium carbonate (8.48 g, 26.0 mmol) in acetonitrile (72.0 mL) was added 2-bromopropane (1.83 mL, 19.5 mmol), and the mixture was stirred at 60° C. for 1 h. After cooling to ambient temperature, the mixture was concentrated in vacuo, and the residue was diluted with ethyl acetate (200 mL) and saturated aqueous sodium bicarbonate solution (200 mL). The aqueous phase was extracted with ethyl acetate (2×200 mL). The organic phase was washed with brine, dried over anhydrous sodium sulfate, filtered, and concentrated in vacuo. Purification of the residue by column chromatography, eluting with a gradient of 0-12% of ethyl acetate in hexanes, afforded the title compound as a colorless oil (2.11 g, 62% yield): ¹H NMR (400 MHz; CDCl₃) δ 7.85 (s, 1H), 4.42 (hept, J=6.7 Hz, 1H), 4.26 (q, J=7.1 Hz, 2H), 1.47 (d, J=6.7 Hz, 6H), 1.31 (t, J=7.1 Hz, 3H); MS (ES-) m/z 260.0 (M−1), 262.0 (M−1).

### Step 3. Preparation of ethyl 3-[(E)-3-[2-[3-amino-2-(3,3-difluoropyrrolidin-1-yl)-4-pyridyl]phenyl]prop-1-enyl]-1-isopropyl-pyrazole-4-carboxylate and ethyl 3-[1-[[2-[3-amino-2-(3,3-difluoropyrrolidin-1-yl)-4-pyridyl]phenyl]methyl]vinyl]-1-isopropyl-pyrazole-4-carboxylate

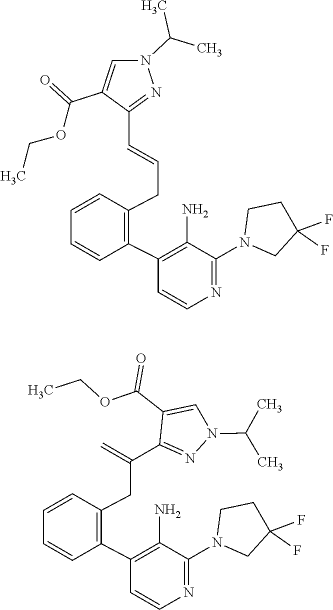 [SMILES: CCOC(=O)c1cn(C(C)C)nc1/C=C/Cc1ccccc1-c1ccnc(N2CCC(F)(F)C2)c1N ; C=C(Cc1ccccc1-c1ccnc(N2CCC(F)(F)C2)c1N)c1nn(C(C)C)cc1C(=O)OCC]

To a solution of 4-(2-allylphenyl)-2-(3,3-difluoropyrrolidin-1-yl)pyridin-3-amine hydrochloride (0.350 g, 0.995 mmol), ethyl 5-bromo-1-isopropyl-pyrazole-4-carboxylate (0.577 g, 1.99 mmol), tris-o-tolylphosphane (0.182 g, 0.597 mmol) and triethylamine (0.555 mL, 3.98 mmol) in N,N-dimethylformamide (6.72 mL) was added palladium(II) acetate (0.0670 g, 0.298 mmol), and the mixture was stirred at 120° C. for 18 h. After cooling to ambient temperature, the mixture was diluted with saturated aqueous sodium bicarbonate (50 mL). The aqueous phase was extracted with ethyl acetate (3×50 mL). The organic layer was washed with brine (50 mL), dried over anhydrous sodium sulfate, filtered, and concentrated in vacuo. Purification of the residue by column chromatography, eluting with a gradient of 0-100% of ethyl acetate in hexanes, afforded the title compounds in a mixture: MS (ES+) m/z 495.8 (M+1).

### Step 4. Preparation of ethyl 3-[3-[2-[3-amino-2-(3,3-difluoropyrrolidin-1-yl)-4-pyridyl]phenyl]propyl]-1-isopropyl-pyrazole-4-carboxylate and ethyl 3-[2-[2-[3-amino-2-(3,3-difluoropyrrolidin-1-yl)-4-pyridyl]phenyl]-1-methyl-ethyl]-1-isopropyl-pyrazole-4-carboxylate

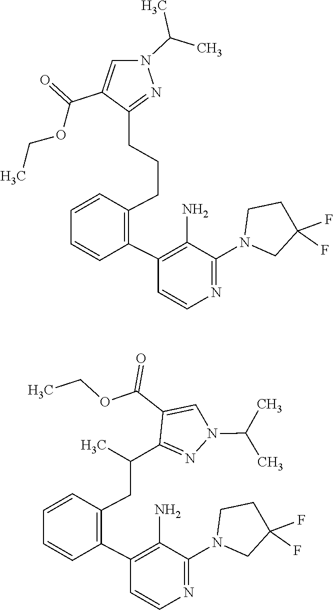 [SMILES: CCOC(=O)c1cn(C(C)C)nc1CCCc1ccccc1-c1ccnc(N2CCC(F)(F)C2)c1N ; CCOC(=O)c1cn(C(C)C)nc1C(C)Cc1ccccc1-c1ccnc(N2CCC(F)(F)C2)c1N]

To a mixture of palladium (10% on carbon matrix, 0.121 g, 0.114 mmol) in methanol (4.37 mL) was added a mixture of ethyl 3-[(E)-3-[2-[3-amino-2-(3,3-difluoropyrrolidin-1-yl)-4-pyridyl]phenyl]prop-1-enyl]-1-isopropyl-pyrazole-4-carboxylate and ethyl 3-[1-[[2-[3-amino-2-(3,3-difluoropyrrolidin-1-yl)-4-pyridyl]phenyl]methyl]vinyl]-1-isopropyl-pyrazole-4-carboxylate (mixture of isomers, 0.125 g, 0.227 mmol) and the mixture was stirred at 22° C. for 1 h 15 minutes under hydrogen. The mixture was diluted with dichloromethane (20 mL) and filtered through a bed of diatomaceous earth (i.e., Celite®). The solid was washed with dichloromethane (200 mL). Concentration of the filtrate in vacuo afforded the title compounds in a mixture: MS (ES+) m/z 498.4 (M+1).

### Step 5. Preparation of lithium;3-[3-[2-[3-amino-2-(3,3-difluoropyrrolidin-1-yl)-4-pyridyl]phenyl]propyl]-1-isopropyl-pyrazole-4-carboxylate and lithium;3-[2-[2-[3-amino-2-(3,3-difluoropyrrolidin-1-yl)-4-pyridyl]phenyl]-1-methyl-ethyl]-1-isopropyl-pyrazole-4-carboxylate

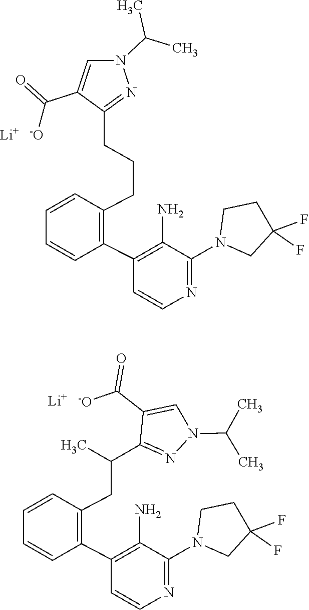

To a solution of ethyl 3-[3-[2-[3-amino-2-(3,3-difluoropyrrolidin-1-yl)-4-pyridyl]phenyl]propyl]-1-isopropyl-pyrazole-4-carboxylate and ethyl 3-[2-[2-[3-amino-2-(3,3-difluoropyrrolidin-1-yl)-4-pyridyl]phenyl]-1-methyl-ethyl]-1-isopropyl-pyrazole-4-carboxylate (mixture of isomers, 0.120 g, 0.241 mmol) in 1,4-dioxane (1.80 mL) and water (1.80 mL) was added lithium hydroxide monohydrate (0.0506 g, 1.21 mmol) and the mixture was stirred at 90° C. for 4 h. After cooling to ambient temperature, the mixture was concentrated in vacuo. Purification of the residue by reverse phase chromatography, eluting with a gradient of 5-100% of acetonitrile in water, containing 10 mM of ammonium bicarbonate, afforded the title compounds in a mixture: MS (ES+) m/z 470.5 (M+1).

### Step 6. Preparation of 6-(3,3-difluoropyrrolidin-1-yl)-12-isopropyl-5,8,12,13-tetrazatetracyclo[16.4.0.02,7.010,14]docosa-1(22),2(7),3,5,10,13,18,20-octaen-9-one, (1 5R)-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9-one,and (15S)-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9-one

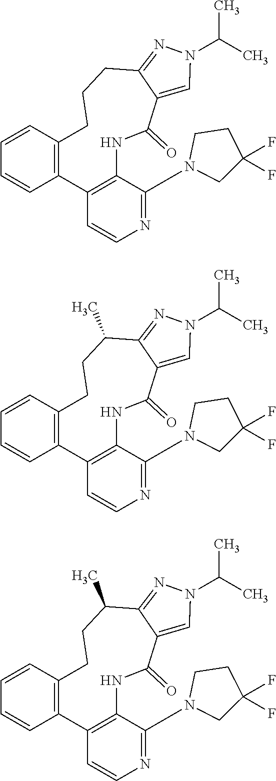 [SMILES: CC(C)n1cc2c(n1)CCCc1ccccc1-c1ccnc(N3CCC(F)(F)C3)c1NC2=O ; CC(C)n1cc2c(n1)[C@@H](C)CCc1ccccc1-c1ccnc(N3CCC(F)(F)C3)c1NC2=O ; CC(C)n1cc2c(n1)[C@H](C)CCc1ccccc1-c1ccnc(N3CCC(F)(F)C3)c1NC2=O]

To a mixture of 2-chloro-1-methyl-pyridin-1-ium;iodide (0.0537 g, 0.210 mmol) in dichloromethane (23.0 mL) was added lithium;3-[3-[2-[3-amino-2-(3,3-difluoropyrrolidin-1-yl)-4-pyridyl]phenyl]propyl]-1-isopropyl-pyrazole-4-carboxylate and lithium;3-[2-[2-[3-amino-2-(3,3-difluoropyrrolidin-1-yl)-4-pyridyl]phenyl]-1-methyl-ethyl]-1-isopropyl-pyrazole-4-carboxylate (mixture of isomers, 0.0750 g, 0.158 mmol), and triethylamine (0.110 mL, 0.0798 mmol) in dichloromethane (7.55 mL) over 4 h at 30° C., and the mixture was stirred at 30° C. for 20 h. After cooling to ambient temperature, the mixture was diluted with saturated aqueous sodium bicarbonate (100 mL), and the aqueous phase was extracted with ethyl acetate (3×100 mL). The organic phase was washed with brine (300 ml), dried over anhydrous sodium sulfate, filtered and concentrated in vacuo. Purification by column chromatography, eluting with a gradient of 0-100% of ethyl acetate in hexanes, followed by preparative reverse phase HPLC, eluting with a gradient of 49-59% of acetonitrile in water, containing 10 mM of ammonium formate, afforded rac-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9-one as a colorless solid (0.025 g), and 6-(3,3-difluoropyrrolidin-1-yl)-12-isopropyl-5,8,12,13-tetrazatetracyclo[16.4.0.02,7.010,14]docosa-1(22),2(7),3,5,10,13,18,20-octaen-9-one as a colorless solid (0.0150 g, 63.2%): ¹H NMR (400 MHz; DMSO-d₆) δ 7.89 (d, J=5.0 Hz, 1H), 7.43 (d, J=7.8 Hz, 1H), 7.37 (td, J=7.6, 1.5 Hz, 1H), 7.29 (s, 1H), 7.27 (t, J=7.4 Hz, 2H), 7.15 (dd, J=7.8, 1.4 Hz, 1H), 6.37 (d, J=4.9 Hz, 1H), 4.25 (p, J=6.7 Hz, 1H), 4.21-4.11 (m, 1H), 3.98-3.86 (m, 2H), 3.75 (t, J=9.7 Hz, 1H), 2.79-2.63 (m, 1H), 2.55-2.51 (m, 1H), 2.48-2.31 (m, 2H), 2.13-2.07 (m, 1H), 1.98-1.88 (m, 2H), 1.74 (t, J=13.7 Hz, 1H), 1.20 (d, J=1.1 Hz, 3H), 1.19 (d, J=1.1 Hz, 3H); MS (ES+) m/z 452.3 (M+1).

Purification of rac-6-(3,3-difluoropyrrolidin-1-yl)-12-isopropyl-15-methyl-5,8,12,13-tetrazatetracyclo[15.4.0.02,7.010,14]henicosa-1(21),2(7),3,5,10,13,17,19-octaen-9-one by chiral SFC, eluting with a gradient of 5-60% of acetonitrile and ethanol in water, containing 10 mM of ammonium formate, afforded the first eluting enantiomer (0.0100 g, 11%) as a colorless solid, and the second eluting enantiomer (0.00800 g, 8%) as a colorless solid: ¹H NMR (400 MHz; DMSO-d₆) δ 9.06-8.45 (m, 1H), 8.45-7.67 (m, 1H), 7.67-6.95 (m, 5H), 6.84-6.33 (m, 1H), 4.42-3.52 (m, 4H), 3.01-2.66 (m, 2H), 2.36-1.87 (m, 2H), 1.54-1.20 (m, 8H), 0.92-0.83 (m, 3H); MS (ES+) m/z 452.3 (M+1).

### Example 199

### Synthesis of N-[2-(3,3-difluoropyrrolidin-1-yl)-4-[4-(1-hydroxy-1-methyl-ethyl)phenyl]-3-pyridyl]-2-isopropyl-pyrimidine-5-carboxamide

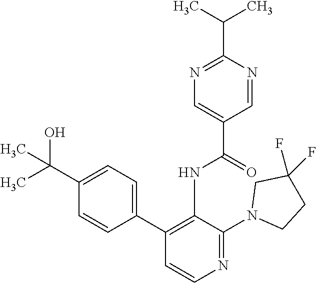 [SMILES: CC(C)c1ncc(C(=O)Nc2c(-c3ccc(C(C)(C)O)cc3)ccnc2N2CCC(F)(F)C2)cn1]

To a mixture of N-[2-(3,3-difluoropyrrolidin-1-yl)-4-iodo-3-pyridyl]-2-isopropyl-pyrimidine-5-carboxamide (0.0500 g, 0.100 mmol), [4-(1-hydroxy-1-methyl-ethyl)phenyl]boronic acid (0.0380 g, 0.201 mmol), and potassium carbonate (0.0347 g, 0.251 mmol) in degassed dioxane (1.20 mL) and water (0.400 mL) was added [1,1′-bis(diphenylphosphino)ferrocene]dichloropalladium(II), complex with dichloromethane (0.0246 g, 0.0301 mmol), and the mixture was stirred at 100° C. for 1 h. After cooling to ambient temperature, the mixture was diluted with ethyl acetate (10 mL), and filtered through a bed of diatomaceous earth (i.e., Celite®). The solid was washed with ethyl acetate (30 mL), and the filtrate was concentrated in vacuo. Purification of the residue by column chromatography, eluting with a gradient of 0-10% of methanol in dichloromethane, followed by preparative reverse phase HPLC, eluting with a gradient of 36-46% acetonitrile in water containing 10 mM of ammonium formate, afforded the title compound as a colorless solid (0.030 g, 62% yield): ¹H NMR (400 MHz; DMSO-d₆) δ 10.07 (s, 1H), 8.86 (s, 2H), 8.13 (d, J=5.0 Hz, 1H), 7.46-7.33 (m, 2H), 7.33-7.18 (m, 2H), 6.75 (d, J=5.0 Hz, 1H), 4.95 (s, 1H), 3.91-3.58 (m, 4H), 3.13 (p, J=6.9 Hz, 1H), 2.44-2.29 (m, 2H), 1.31 (s, 6H), 1.22 (d, J=6.9 Hz, 6H); MS (ES+) m/z 482.3 (M+1).

### Example 200

### Synthesis of N-[2-(3,3-difluoropyrrolidin-1-yl)-4-[3-(1-hydroxy-1-methyl-ethyl)phenyl]-3-pyridyl]-2-isopropyl-pyrimidine-5-carboxamide

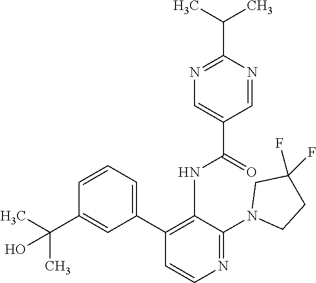 [SMILES: CC(C)c1ncc(C(=O)Nc2c(-c3cccc(C(C)(C)O)c3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0500 g, 0.106 mmol), [3-(1-hydroxy-1-methyl-ethyl)phenyl]boronic acid (0.0285 g, 0.158 mmol) and [1,1′ bis(diphenylphosphino)ferrocene]dichloropalladium(II), complex with dichloromethane (0.0173 g, 0.0211 mmol) in 1,4-dioxane (1.00 mL) and water (0.200 mL) was added potassium carbonate (0.0365 g, 0.264 mmol), and the mixture was stirred at 100° C. for 2 h. After cooling to ambient temperature, the mixture was diluted with ethyl acetate (25 mL), and passed through a bed of diatomaceous earth (i.e., Celite®). The solid was washed with ethyl acetate (50 mL) and the filtrate was concentrated in vacuo. Purification of the residue by reverse phase chromatography, eluting with a gradient of 15-100% of acetonitrile in water containing 10 mM of ammonium formate followed by preparative reverse phase HPLC, eluting with a gradient of 36-46% of acetonitrile in water containing 10 mM of ammonium formate, afforded the title compound as a colorless solid (0.0250 g, 49% yield): ¹H NMR (500 MHz; DMSO-d₆) δ 10.14 (s, 1H), 8.95 (s, 2H), 8.19 (d, J=4.9 Hz, 1H), 7.48 (t, J=1.8 Hz, 1H), 7.44-7.38 (m, 1H), 7.33 (t, J=7.7 Hz, 1H), 7.21-7.15 (m, 1H), 6.80 (d, J=5.0 Hz, 1H), 4.97 (s, 1H), 4.08-3.61 (m, 4H), 3.22-3.13 (m, 1H), 2.50-2.41 (m, 2H), 1.26 (d, J=6.9 Hz, 6H), 1.25 (s, 6H); ¹⁹F NMR (376 MHz; DMSO-d₆) 3-101.14 (d, J=180.8 Hz); MS (ES+) m/z 482.3 (M+1).

### Example 201

### Synthesis of N-[2-(3,3-difluoropyrrolidin-1-yl)-4-(2-fluorophenyl)-3-pyridyl]-1,3-dihydropyrrolo[3,4-c]pyridine-2-carboxamide

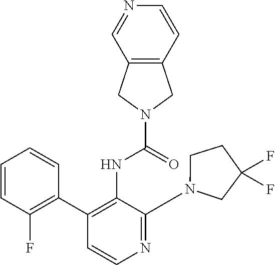 [SMILES: O=C(Nc1c(-c2ccccc2F)ccnc1N1CCC(F)(F)C1)N1Cc2ccncc2C1]

To a solution of 2-(3,3-difluoropyrrolidin-1-yl)-4-(2-fluorophenyl)pyridin-3-amine (0.0500 g, 0.136 mmol) in tetrahydrofuran (1.00 mL) was added triphosgene (0.00267 g, 0.0900 mmol), and the mixture was stirred at 0° C. for 2 h. A mixture of 2,3-dihydro-1H-pyrrolo[3,4-c]pyridin-2-ium chloride (0.0427 g, 0.273 mmol) and N,N-diisopropylethylamine (0.117 mL, 0.682 mmol) in N,N-dimethylformamide (1.00 mL) was added and the mixture was stirred at 22° C. for 1 h. The mixture was diluted with ethyl acetate (50 mL), and the organic phase was washed with water (50 mL). The organic phase was dried over anhydrous sodium sulfate, filtered, and concentrated in vacuo. Purification of the residue by column chromatography, eluting with a gradient of 15-100% of ethyl acetate in hexanes, followed by purification of the residue by reverse phase chromatography, eluting with a gradient of 15-100% of acetonitrile in water containing 10 mM of ammonium bicarbonate, afforded the title compound as a colorless solid (0.0100 g, 16% yield): ¹H NMR (400 MHz; DMSO-d₆) 58.47 (d, J=1.1 Hz, 1H), 8.41 (d, J=5.0 Hz, 1H), 8.07 (d, J=5.0 Hz, 1H), 7.88 (s, 1H), 7.33-7.21 (m, 3H), 7.17 (ddd, J=9.7, 8.3, 1.2 Hz, 1H), 7.09 (td, J=7.5, 1.2 Hz, 1H), 6.68 (dd, J=5.0, 1.1 Hz, 1H), 4.41 (d, J=9.6 Hz, 4H), 3.93 (t, J=13.6 Hz, 2H), 3.77 (t, J=7.3 Hz, 2H), 2.46-2.31 (m, 2H); ¹⁹F NMR (376 MHz; DMSO-d₆) δ-100.65, -114.43; MS (ES+) m/z 440.2.

### Example 202

### Synthesis of N-[2-(3,3-difluoropyrrolidin-1-yl)-4-(2-fluorophenyl)-3-pyridyl]-5-methoxy-isoindoline-2-carboxamide

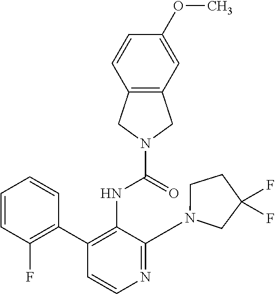 [SMILES: COc1ccc2c(c1)CN(C(=O)Nc1c(-c3ccccc3F)ccnc1N1CCC(F)(F)C1)C2]

To a solution of 2-(3,3-difluoropyrrolidin-1-yl)-4-(2-fluorophenyl)pyridin-3-amine (0.050 g, 0.136 mmol) in tetrahydrofuran (1.00 mL) was added triphosgene (0.0267 g, 0.0900 mmol), and the mixture was stirred at 0° C. for 2 h. A mixture of 5-methoxyisoindoline hydrochloride (0.0506 g, 0.273 mmol) and N,N-diisopropylethylamine (0.117 mL, 0.682 mmol) in tetrahydrofuran (1.00 mL) was added and the mixture was stirred at 22° C. for 1 h. The mixture was diluted with ethyl acetate (50 mL), and the organic phase was washed with water (50 mL). The organic phase was dried over anhydrous sodium sulfate, filtered, and concentrated in vacuo. Purification of the residue by column chromatography, eluting with a gradient of 15-100% of ethyl acetate in hexanes, followed by purification of the residue by reverse phase chromatography, eluting with a gradient of 15-100% of acetonitrile in water containing 10 mM of ammonium bicarbonate, afforded the title compound as a colorless solid (0.0150 g, 23% yield): ¹H NMR (300 MHz; DMSO-d₆) δ 8.12 (d, J=5.0 Hz, 1H), 7.81 (s, 1H), 7.39-7.26 (m, 2H), 7.26-7.03 (m, 3H), 6.84 (d, J=7.8 Hz, 2H), 6.77-6.69 (m, 1H), 4.37 (d, J=12.0 Hz, 4H), 3.99 (t, J=13.6 Hz, 2H), 3.83 (t, J=7.2 Hz, 2H), 3.74 (s, 3H), 2.42 (dt, J=14.1, 7.1 Hz, 2H); ⁹F NMR (376 MHz; DMSO-d₆) (−100.65, -114.46; MS (ES+) m/z 469.2.

### Example 203

### Synthesis of tert-butyl 6-[[2-(3,3-difluoropyrrolidin-1-yl)-4-(2-fluorophenyl)-3-pyridyl]carbamoyloxy]-2-azaspiro[3.3]heptane-2-carboxylate

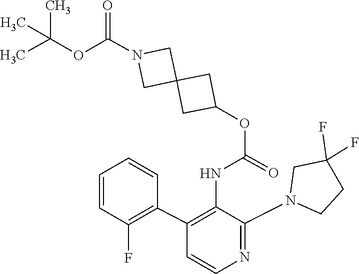 [SMILES: CC(C)(C)OC(=O)N1CC2(CC(OC(=O)Nc3c(-c4ccccc4F)ccnc3N3CCC(F)(F)C3)C2)C1]

To a solution of 2-(3,3-difluoropyrrolidin-1-yl)-4-(2-fluorophenyl)pyridin-3-amine (0.200 g, 0.546 mmol) in tetrahydrofuran (2.00 mL) was added triphosgene (0.107 mg, 0.360 mmol), and the mixture was stirred at 0° C. for 2 h. A mixture of tert-butyl 6-hydroxy-2-azaspiro[3.3]heptane-2-carboxylate (0.233, 1.09 mmol) and N,N-diisopropylethylamine (0.374 mL, 2.18 mmol) in tetrahydrofuran (1.00 mL) was added and the mixture was stirred at 22° C. for 2 h. N,N-dimethylformamide (1.00 mL) was added and the mixture was stirred at 22° C. for 16 h. The mixture was diluted with ethyl acetate (50 mL), and the organic phase was washed with water (50 mL). The organic phase was dried over anhydrous sodium sulfate, filtered, and concentrated in vacuo. Purification of the residue by column chromatography, eluting with a gradient of 15-100% of ethyl acetate in hexanes, afforded the title compound as a colorless solid (80% pure, 0.227 g, 62% yield). Purification of the residue (0.0400 g) by preparative reverse phase HPLC, eluting with a gradient of 60-70% of acetonitrile in water containing 10 mM of ammonium formate, afforded the title compound as a colorless solid (0.010 g, 25% yield): ¹H NMR (400 MHz; DMSO-d₆) δ 8.72 (s, 0.7H, rotamer), 8.52 (s, 0.3H, rotamer), 8.11 (d, J=5.0 Hz, 1H), 7.51-7.34 (m, 1H), 7.33-7.09 (m, 3H), 6.71 (d, J=5.1 Hz, 1H), 4.49 (q, J=6.8 Hz, 0.7H, rotamer), 4.40-4.33 (m, 0.3H, rotamer), 3.96-3.58 (m, 8H), 2.49-2.40 (m, 2H), 2.40-2.34 (m, 2H), 1.85 (bs, 2H), 1.35 (s, 9H); ¹⁹F NMR (376 MHz; DMSO-d₆) δ -100.60 (t, J=13.9 Hz), −114.62; MS (ES+) m/z 533.3.

### Example 204

### Synthesis of N-[2-(3,3-difluoropyrrolidin-1-yl)-4-(3-fluoro-2-pyridyl)-3-pyridyl]-2-isopropyl-pyrimidine-5-carboxamide

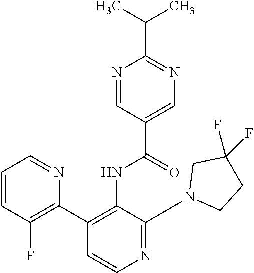 [SMILES: CC(C)c1ncc(C(=O)Nc2c(-c3ncccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.250 g, 0.528 mmol), tributyl-(3-fluoro-2-pyridyl)stannane (0,245 mg, 0.634 mmol), and 2-dicyclohexylphosphino-2′,4′,6′-triisopropylbiphenyl (0.137 g, 0.317 mmol) in 1,4-dioxane (2.00 mL) was added palladium(II) acetate (0.0356 g, 0.158 mmol), and the mixture was stirred at 100° C. for 16 h. After cooling to ambient temperature, the mixture was diluted with ethyl acetate (10 mL). The mixture was filtered through diatomaceous earth (i.e., Celite®) washing with ethyl acetate (20 mL) and the filtrate was concentrated in vacuo. Purification of the residue by column chromatography, eluting with a gradient of 0-60% of acetone in dichloromethane, followed by purification by reverse phase chromatography, eluting with a gradient of 34-44% of acetonitrile in water containing 10 mM of ammonium formate, afforded the title compound as a yellow solid (0.0360 g, 15% yield): ¹H NMR (400 MHz; DMSO-d₆) δ 10.25 (s, 1H), 8.90 (s, 2H), 8.43 (dt, J=4.6, 1.5 Hz, 1H), 8.25 (d, J=4.9 Hz, 1H), 7.81-7.75 (m, 1H), 7.45 (dt, J=8.5, 4.3 Hz, 1H), 6.92 (dd, J=4.9, 1.3 Hz, 1H), 3.91 (t, J=13.3 Hz, 2H), 3.76 (t, J=7.3 Hz, 2H), 3.22-3.11 (m, 1H), 2.49-2.36 (m, 2H), 1.27 (d, J=6.9 Hz, 6H); ¹⁹F NMR (376 MHz; DMSO-d₆) δ-101.08 (t, J=13.7 Hz), −120.82 (d, J=10.5 Hz); MS (ES+) m/z 443.3 (M+1).

### Example 205

### Synthesis of N-[2-(3,3-difluoropyrrolidin-1-yl)-4-(6-methoxy-2-pyridyl)-3-pyridyl]-2-isopropyl-pyrimidine-5-carboxamide

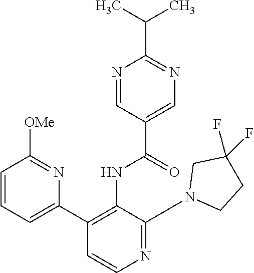 [SMILES: COc1cccc(-c2ccnc(N3CCC(F)(F)C3)c2NC(=O)c2cnc(C(C)C)nc2)n1]

To a mixture of N-[2-(3,3-difluoropyrrolidin-1-yl)-4-iodo-3-pyridyl]-2-isopropyl-pyrimidine-5-carboxamide (0.0590 g, 0.125 mmol), 2-methoxy-6-(4,4,5,5-tetramethyl-1,3,2-dioxaborolan-2-yl)pyridine (0.0449 g, 0.187 mmol) and potassium carbonate (0.0533 g, 0.386 mmol) in 1,4-dioxane (0.960 mL) and water (0.240 mL) was added methanesulfonato(tri-t-butylphosphino)(2′-amino-1,1′-biphenyl-2-yl)palladium(II) (0.0145 g, 0.0248 mmol), and the mixture was stirred at 90° C. for 1 h.

After cooling to ambient temperature, the mixture was diluted with brine (5 mL) and ethyl acetate (25 mL). The aqueous phase was extracted with ethyl acetate (2×25 mL). The combined organic phases were dried over sodium sulfate, filtered and concentrated in vacuo. Purification of the residue by column chromatography, eluting with a gradient of 0-40% of ethyl acetate in hexanes, followed by reverse phase chromatography, eluting with a gradient of 10-100% of acetonitrile in water containing 10 mM of ammonium formate, afforded the title compound as a colorless solid (0.0152 g, 27% yield): ¹H NMR (400 MHz; DMSO-d₆) δ 10.19 (s, 1H), 9.02 (s, 2H), 8.23 (d, J=5.0 Hz, 1H), 7.72 (dd, J=8.3, 7.3 Hz, 1H), 7.16 (dd, J=7.3, 0.8 Hz, 1H), 6.99 (d, J=5.0 Hz, 1H), 6.76 (dd, J=8.4, 0.7 Hz, 1H), 3.90 (s, 2H), 3.77 (s, 5H), 3.19 (hept, J=6.9 Hz, 1H), 2.43 (dq, J=14.3, 7.2 Hz, 2H), 1.29 (d, J=6.9 Hz, 6H); ¹⁹F NMR (376 MHz, DMSO-d₆) δ-101.30-101.0 (m); MS (ES+) m/z 455.3 (M+1).

### Example 206

### Synthesis of N-[2-(3,3-difluoropyrrolidin-1-yl)-4-[6-(trifluoromethyl)-2-pyridyl]-3-pyridyl]-2-isopropyl-pyrimidine-5-carboxamide

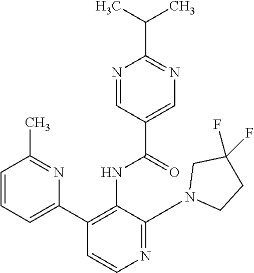 [SMILES: Cc1cccc(-c2ccnc(N3CCC(F)(F)C3)c2NC(=O)c2cnc(C(C)C)nc2)n1]

To a mixture of N-[2-(3,3-difluoropyrrolidin-1-yl)-4-iodo-3-pyridyl]-2-isopropyl-pyrimidine-5-carboxamide (0.101 g, 0.213 mmol), 2-(4,4,5,5-tetramethyl-1,3,2-dioxaborolan-2-yl)-6-(trifluoromethyl)pyridine (0.0873 g, 0.313 mmol) and potassium carbonate (0.0929 g, 0.672 mmol) in 1,4-dioxane (1.60 mL) and water (0.400 mL) was added methanesulfonato(tri-t-butylphosphino)(2′-amino-1,1′-biphenyl-2-yl)palladium(II) (0.0302 g, 0.0517 mmol), and the mixture was stirred at 90° C. for 1 h. After cooling to ambient temperature, the mixture was filtered washing with ethyl acetate (15 mL) and concentrated in vacuo. Purification of the residue by column chromatography, eluting with a gradient of 0-10% of methanol in dichloromethane, followed by reverse phase chromatography, eluting with a gradient of 20-100% of acetonitrile in water containing 10 mM of ammonium formate, afforded the title compound as an yellow solid (0.0735 g, 70% yield): ¹H NMR (500 MHz; DMSO-d₆) δ 10.28 (s, 1H), 9.00 (s, 2H), 8.28 (d, J=5.0 Hz, 1H), 8.14 (t, J=7.9 Hz, 1H), 7.89-7.81 (m, 2H), 6.99 (d, J=5.0 Hz, 1H), 3.92 (s, 2H), 3.77 (s, 2H), 3.18 (hept, J=6.9 Hz, 1H), 2.48-2.38 (m, 2H), 1.27 (d, J=6.9 Hz, 6H); ¹⁹F NMR (376 MHz, DMSO-d₆) δ-66.54 (s),-101.15 (s); MS (ES+) m/z 493.2 (M+1).

### Example 207

### Synthesis of N-[2-(3,3-difluoropyrrolidin-1-yl)-4-(3-fluoro-2-pyridyl)-3-pyridyl]-2-isopropoxy-pyrimidine-5-carboxamide

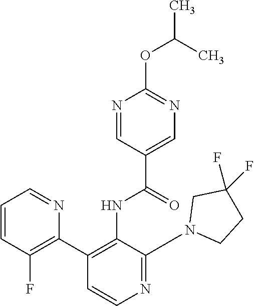 [SMILES: CC(C)Oc1ncc(C(=O)Nc2c(-c3ncccc3F)ccnc2N2CCC(F)(F)C2)cn1]

To a solution of N-[2-(3,3-difluoropyrrolidin-1-yl)-4-iodo-3-pyridyl]-2-isopropoxy-pyrimidine-5-carboxamide (0.100 g, 0.204 mmol), tributyl-(3-fluoro-2-pyridyl)stannane (0.158 g, 0.409 mmol), and 2-dicyclohexylphosphino-2′,4′,6′-triisopropylbiphenyl (0.0532 g, 0.123 mmol) in 1,4-dioxane (2.00 mL) was added palladium(II) acetate (0.0138 g, 0.0613 mmol), and the mixture was stirred at 100° C. for 20 h. After cooling to ambient temperature, the mixture was diluted with ethyl acetate (10 mL). The mixture was filtered through diatomaceous earth (i.e., Celite®) washing with ethyl acetate (20 mL) and the filtrate was concentrated in vacuo. Purification of the residue by column chromatography, eluting with a gradient of 0-60% of acetone in dichloromethane, followed by purification by reverse phase chromatography, eluting with a gradient of 20-50% of acetonitrile in water containing 10 mM of ammonium formate, afforded the title compound as a colorless solid (0.0120 g, 13% yield): ¹H NMR (400 MHz; DMSO-d₆) δ 10.10 (br s, 1H), 8.79 (s, 2H), 8.41 (dt, J=4.6, 1.5 Hz, 1H), 8.24 (d, J=5.0 Hz, 1H), 7.77 (ddd, J=10.0, 8.5, 1.3 Hz, 1H), 7.43 (dt, J=8.5, 4.3 Hz, 1H), 6.91 (dd, J=4.9, 1.3 Hz, 1H), 5.24 (p, J=6.2 Hz, 1H), 3.90 (t, J=13.3 Hz, 2H), 3.75 (t, J=7.3 Hz, 2H), 2.42 (dq, J=14.3, 7.2 Hz, 2H), 1.32 (d, J=6.2 Hz, 6H); ¹⁹F NMR (376 MHz; DMSO-d₆) b -100.92-101.09 (m), −120.78 (d, J=7.0 Hz); MS (ES+) m/z 459.2 (M+1).

### Example 208

### Synthesis of 6-(3,3-difluoropyrrolidin-1-yl)-13-methoxy-5,8,12,14-tetrazatetracyclo[16.4.0.02,7.010,15]docosa-1(22),2(7),3,5,10(15),11,13,18,20-nonaen-9-one

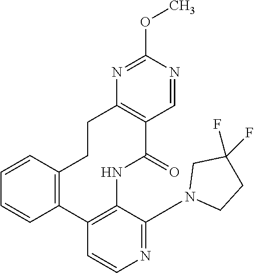 [SMILES: COc1ncc2c(n1)CCc1ccccc1-c1ccnc(N3CCC(F)(F)C3)c1NC2=O]

### Step 1. Preparation of ethyl 4-[2-(2-chlorophenyl)ethynyl]-2-methoxy-pyrimidine-5-carboxylate

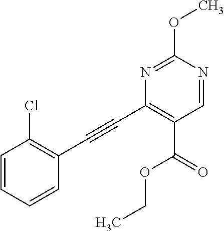 [SMILES: CCOC(=O)c1cnc(OC)nc1C#Cc1ccccc1Cl]

To a solution of ethyl 4-chloro-2-methoxy-pyrimidine-5-carboxylate (0.317 g, 1.46 mmol), 1-chloro-2-ethynyl-benzene (0.0889 mL, 0.732 mmol), copper(I) iodide (0.0209 g, 0.110 mmol) and triethylamine (0.307 mL, 2.20 mmol) in acetonitrile (1.00 mL) was added tetrakis(triphenylphosphine)palladium(0) (0.00423 g, 0.00366 mmol), and the mixture was stirred at 70° C. for 50 min. After cooling to ambient temperature, the mixture was diluted with ethyl acetate (5.00 mL). The organic phase was concentrated in vacuo. Purification of the residue by column chromatography, eluting with a gradient of 0-50% of ethyl acetate in hexanes, afforded the title compound as an orange oil (0.114 g, 49% yield): ¹H NMR (300 MHz; CDC₃) δ 9.12 (s, 1H), 7.70 (dd, J=7.5, 1.9 Hz, 1H), 7.46 (dd, J=7.9, 1.4 Hz, 1H), 7.36 (td, J=7.7, 1.9 Hz, 1H), 7.33-7.26 (m, 1H), 4.43 (q, J=7.1 Hz, 2H), 4.11 (s, 3H), 1.39 (t, J=7.1 Hz, 3H); MS (ES+) m/z 318.3 (M+1), 320.1 (M+1)

### Step 2. Preparation of ethyl 4-[2-(2-chlorophenyl)ethyl]-2-methoxy-pyrimidine-5-carboxylate

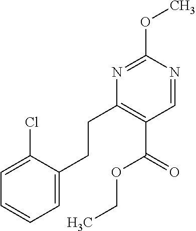 [SMILES: CCOC(=O)c1cnc(OC)nc1CCc1ccccc1Cl]

To a solution of ethyl 4-[2-(2-chlorophenyl)ethynyl]-2-methoxy-pyrimidine-5-carboxylate (0.114 g, 0.360 mmol) in dichloromethane (1.00 mL) and methanol (1.00 mL) was added palladium (10% on matrix carbon, 0.0192 g, 0.281 mmol), and the mixture was stirred at 23° C. for 2 h under hydrogen. The mixture was passed through a bed of diatomaceous earth (i.e., Celite®). The solid was washed with dichloromethane (25 mL) and the filtrate was concentrated in vacuo. Purification of the residue by column chromatography, eluting with a gradient of 0-40% of ethyl acetate in hexanes, afforded the title compound as a yellow solid (0.0900 g, 78% yield): ¹H NMR (300 MHz; CDCl₃) δ 9.00 (d, J=3.1 Hz, 1H), 7.36-7.31 (m, 1H), 7.25-7.20 (m, 1H), 7.17-7.12 (m, 2H), 4.34 (q, J=7.2 Hz, 2H), 4.04 (s, 3H), 3.51-3.42 (m, 2H), 3.20 (dd, J=9.4, 6.3 Hz, 2H), 1.38 (td, J=7.1, 4.3 Hz, 3H); MS (ES+) m/z 321.6 (M+1), 323.3 (M+1).

### Step 3. Preparation of ethyl 2-methoxy-4-[2-[2-(4,4,5,5-tetramethyl-1,3,2-dioxaborolan-2-yl)phenyl]ethyl]pyrimidine-5-carboxylate

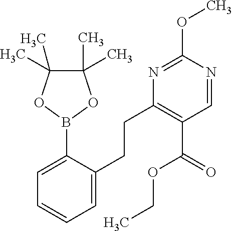 [SMILES: CCOC(=O)c1cnc(OC)nc1CCc1ccccc1B1OC(C)(C)C(C)(C)O1]

To a mixture of ethyl 4-[2-(2-chlorophenyl)ethyl]-2-methoxy-pyrimidine-5-carboxylate (0.600 g, 1.87 mmol), 4,4,4′,4′,5,5,5′,5′-octamethyl-2,2′-bi-1,3,2-dioxaborolane (0.522 g, 2.06 mmol), potassium acetate (0.275 g, 2.81 mmol), tris(dibenzylideneacetone)dipalladium(0) (0.0269 g, 0.0468 mmol) and [2-(2-methoxyphenyl)-1-methyl-indol-3-yl]-diphenyl-phosphane (0.0788 g, 0.187 mmol) was added 1,4-dioxane (5.00 mL), and the mixture was stirred at 110° C. for 2 h. After cooling to ambient temperature, the mixture was passed through a bed of diatomaceous earth (i.e., Celite®). The solid was washed with ethyl acetate (50 mL) and the filtrate was concentrated in vacuo. Purification of the residue by column chromatography, eluting with a gradient of 0-25% of ethyl acetate in hexanes, afforded the title compound as a colorless oil (75% pure, 0.617 g, 60% yield): ¹H NMR (300 MHz; CDCl₃) δ 8.94 (s, 1H), 7.77 (dd, J=7.6, 1.5 Hz, 1H), 7.35-7.26 (m, 1H), 7.20-7.12 (m, 2H), 4.33-4.25 (m, 2H), 4.02 (s, 3H), 3.52-3.41 (m, 2H), 3.39-3.31 (m, 2H), 1.37-1.33 (m, 3H), 1.31 (s, 12H); MS (ES+) m/z 413.2 (M+1).

### Step 4. Preparation of ethyl 4-[2-[2-[3-amino-2-(3,3-difluoropyrrolidin-1-yl)-4-pyridyl]phenyl]ethyl]-2-methoxy-pyrimidine-5-carboxylate

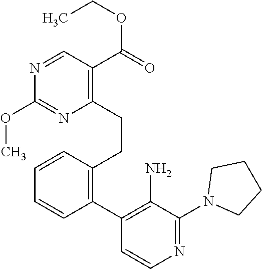 [SMILES: CCOC(=O)c1cnc(OC)nc1CCc1ccccc1-c1ccnc(N2CCCC2)c1N]

To a solution of 2-(3,3-difluoropyrrolidin-1-yl)-4-iodo-pyridin-3-amine;hydrochloride (0.350 g, 0.920 mmol), ethyl 2-methoxy-4-[2-[2-(4,4,5,5-tetramethyl-1,3,2-dioxaborolan-2-yl)phenyl]ethyl]pyrimidine-5-carboxylate (0.607 g, 1.10 mmol), and potassium carbonate (0.445 g, 3.22 mmol) in 1,4-dioxane (9.00 mL) and water (3.00 mL) was added [1,1′-bis(diphenylphosphino)ferrocene]dichloropalladium(II), complex with dichloromethane (0.225 g, 0.276 mmol), and the mixture was stirred at 90° C. for 60 minutes. After cooling to ambient temperature, the mixture was diluted with saturated aqueous sodium bicarbonate (50 mL), and the aqueous phase was extracted with ethyl acetate (3×50 mL). The organic phase was washed with brine (100 mL), dried over anhydrous sodium sulfate and concentrated in vacuo. The residue was purified by column chromatography, eluting with a gradient of 0-60% of ethyl acetate in hexanes, afforded the title compound as a yellow oil (0.417 g, 94% yield): ¹H NMR (300 MHz; CDCl₃) δ 8.92 (s, 1H), 7.77 (d, J=5.0 Hz, 1H), 7.37-7.27 (m, 3H), 7.19-7.13 (m, 1H), 6.69 (d, J=4.9 Hz, 1H), 4.28 (q, J=7.1 Hz, 2H), 3.92 (s, 3H), 3.78-3.61 (m, 2H), 3.61-3.49 (m, 4H), 3.42-3.18 (m, 2H), 3.05-2.81 (m, 2H), 2.53-2.32 (m, 2H), 1.35 (t, J=7.1 Hz, 3H); MS (ES+) m/z 484.1 (M+1).

### Step 5. Preparation of lithium;4-[2-[2-[3-amino-2-(3,3-difluoropyrrolidin-1-yl)-4-pyridyl]phenyl]ethyl]-2-methoxy-pyrimidine-5-carboxylate

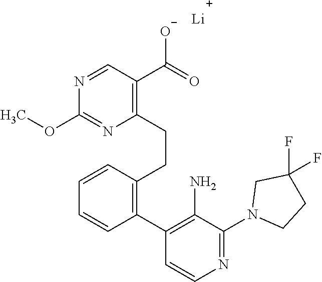 [SMILES: [Li+] ; COc1ncc(C(=O)[O-])c(CCc2ccccc2-c2ccnc(N3CCC(F)(F)C3)c2N)n1]

To a solution of ethyl 4-[2-[2-[3-amino-2-(3,3-difluoropyrrolidin-1-yl)-4-pyridyl]phenyl]ethyl]-2-methoxy-pyrimidine-5-carboxylate (0.400 g, 0.827 mmol) in 1,4-dioxane (10.0 mL) and water (10.0 mL) was added lithium hydroxide monohydrade (0.174 g, 4.14 mmol) and the mixture was stirred at 90° C. for 1 h. After cooling to ambient temperature, the mixture was concentrated in vacuo. Purification of the residue by reverse phase chromatography, eluting with a gradient of 10-100% of acetonitrile in water, containing 10 mM of ammonium formate, afforded the title compound as colorless solid (0.180 g, 47% yield): ¹H NMR (300 MHz; DMSO-d₆) δ 8.71 (s, 1H), 7.61 (d, J=4.9 Hz, 1H), 7.39-7.25 (m, 3H), 7.13-7.08 (m, 1H), 6.64 (d, J=4.9 Hz, 1H), 4.14 (s, 2H), 3.74 (s, 3H), 3.68 (dd, J=13.7, 8.1 Hz, 2H), 3.47 (td, J=7.1, 3.1 Hz, 2H), 3.35 (ddd, J=13.2, 9.9, 5.8 Hz, 1H), 3.19 (ddd, J=13.3, 9.6, 6.4 Hz, 1H), 2.92-2.69 (m, 2H), 2.41 (dq, J=14.5, 7.3 Hz, 2H); MS (ES-) m/z 454.3 (M−1).

### Step 6. Preparation of 6-(3,3-difluoropyrrolidin-1-yl)-13-methoxy-5,8,12,14-tetrazatetracyclo[16.4.0.02,7.010,15]docosa-1(22),2(7),3,5,10(15),11,13,18,20

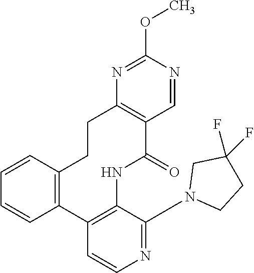 [SMILES: COc1ncc2c(n1)CCc1ccccc1-c1ccnc(N3CCC(F)(F)C3)c1NC2=O]

To a mixture of 2-chloro-1-methyl-pyridin-1-ium iodide (0.177 g, 0.694 mmol) in dichloromethane (55.0 mL) was added lithium;4-[2-[2-[3-amino-2-(3,3-difluoropyrrolidin-1-yl)-4-pyridyl]phenyl]ethyl]-2-methoxy-pyrimidine-5-carboxylate (0.0800 g, 0.173 mmol) and triethylamine (0.121 mL, 0.867 mmol) in dichloromethane (15.0 mL) over 5 h at 30° C., and the mixture was stirred at 30° C. for 18 h. After cooling to ambient temperature, the mixture was diluted with saturated aqueous sodium bicarbonate (100 mL), and the aqueous phase was extracted with ethyl acetate (3×100 mL). The organic phase was washed with brine (200 ml), dried over anhydrous sodium sulfate, filtered and concentrated in vacuo. Purification by column chromatography, eluting with a gradient of 0-100% of ethyl acetate in hexanes, followed by reverse phase chromatography, eluting with a gradient of 5-100% of acetonitrile in water containing 10 mM of ammonium bicarbonate, afforded the title compound as a colorless solid (0.0130 g, 17% yield): ¹H NMR (300 MHz; DMSO-d₆) δ 9.60 (s, 1H), 8.39 (s, 1H), 7.91 (d, J=4.9 Hz, 1H), 7.47-7.29 (m, 3H), 7.08 (d, J=7.1 Hz, 1H), 6.56 (d, J=4.9 Hz, 1H), 4.22-3.83 (m, 2H), 3.82 (s, 3H), 3.62-3.36 (m, 2H), 3.26-3.02 (m, 4H), 2.31-2.11 (m, 2H); MS (ES+) m/z 438.1 (M+1).

### Example 209

### Synthesis of N-[2-(3,3-difluoropyrrolidin-1-yl)-4-(3-fluoro-2-pyridyl)-3-pyridyl]-6-isopropoxy-pyridine-3-carboxamide

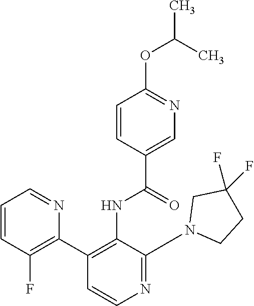 [SMILES: CC(C)Oc1ccc(C(=O)Nc2c(-c3ncccc3F)ccnc2N2CCC(F)(F)C2)cn1]

To a solution of N-[2-(3,3-difluoropyrrolidin-1-yl)-4-iodo-3-pyridyl]-6-isopropoxy-pyridine-3-carboxamide (0.100 g, 0.205 mmol), tributyl-(3-fluoro-2-pyridyl)stannane (0.158 g, 0.410 mmol), and 2-dicyclohexylphosphino-2′,4′,6′-triisopropylbiphenyl (0.0533 g, 0.123 mmol) in 1,4-dioxane (2.00 mL) was added palladium(II) acetate (0.0138 g, 0.0614 mmol), and the mixture was stirred at 100° C. for 20 h. After cooling to ambient temperature, the mixture was diluted with ethyl acetate (10 mL). The mixture was passed through diatomaceous earth (i.e., Celite®). The solid was washed with ethyl acetate (20 mL) and the filtrate was concentrated in vacuo. Purification of the residue by reverse phase chromatography, eluting with a gradient of 15-60% of acetonitrile in water containing 10 mM of ammonium formate, followed by purification of the residue by preparative reverse phase HPLC, eluting with a gradient of 41-51% of acetonitrile in water containing 10 mM of ammonium formate, afforded the title compound as a yellow solid (0.00800 g, 9% yield): ¹H NMR (400 MHz; DMSO-d₆), 9.85 (s, 1H), 8.43 (dd, J=2.6, 0.8 Hz, 1H), 8.37 (dt, J=4.6, 1.6 Hz, 1H), 8.19 (d, J=5.0 Hz, 1H), 7.87 (dd, J=8.6, 2.6 Hz, 1H), 7.71 (ddd, J=10.0, 8.5, 1.3 Hz, 1H), 7.38 (dt, J=8.5, 4.3 Hz, 1H), 6.85 (dd, J=4.9, 1.2 Hz, 1H), 6.72 (dd, J=8.7, 0.7 Hz, 1H), 5.23 (p, J=6.2 Hz, 1H), 3.86 (t, J=13.4 Hz, 2H), 3.72 (t, J=7.3 Hz, 2H), 2.38 (dq, J=14.3, 7.2 Hz, 2H), 1.25 (d, J=6.2 Hz, 6H); ¹⁹F NMR (376 MHz; DMSO-d₆) δ -101.02 (s), −120.68 (s) (d, J=10.4 Hz); MS (ES+) m/z 458.2 (M+1).

### Example 210

### Synthesis of N-[2-(3,3-difluoropyrrolidin-1-yl)-4-(2-pyridyl)-3-pyridyl]-2-isopropoxy-pyrimidine-5-carboxamide

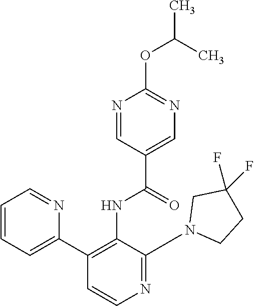 [SMILES: CC(C)Oc1ncc(C(=O)Nc2c(-c3ccccn3)ccnc2N2CCC(F)(F)C2)cn1]

To a solution of N-[2-(3,3-difluoropyrrolidin-1-yl)-4-iodo-3-pyridyl]-2-isopropoxy-pyrimidine-5-carboxamide (0.100 g, 0.204 mmol), tributyl(2-pyridyl)stannane (0.0993 mL, 0.409 mmol), and 2-dicyclohexylphosphino-2′,4′,6′-triisopropylbiphenyl (0.0532 g, 0.123 mmol) in 1,4-dioxane (2.00 mL) was added palladium(II) acetate (0.0138 g, 0.0613 mmol), and the mixture was stirred at 100° C. for 20 h. After cooling to ambient temperature, the mixture was diluted with ethyl acetate (10 mL). The mixture was filtered through diatomaceous earth (i.e., Celite®) washing with ethyl acetate (20 mL) and the filtrate was concentrated in vacuo. Purification of the residue by reverse phase chromatography, eluting with a gradient of 20-50% of acetonitrile in water containing 10 mM of ammonium formate, followed by purification by preparative reverse phase HPLC, eluting with a gradient of 39-49% of acetonitrile in water containing 10 mM of ammonium formate, afforded the title compound as a colorless solid (0.00700 g, 8% yield): ¹H NMR (400 MHz; DMSO-d₆) δ 10.15 (s, 1H), 8.88 (s, 2H), 8.60 (ddd, J=4.8, 1.8, 1.0 Hz, 1H), 8.23 (d, J=5.0 Hz, 1H), 7.82 (td, J=7.8, 1.9 Hz, 1H), 7.56 (dt, J=7.9, 1.1 Hz, 1H), 7.34 (ddd, J=7.6, 4.8, 1.1 Hz, 1H), 6.96 (d, J=5.0 Hz, 1H), 5.32-2.20 (m, 1H), 3.97-2.81 (m, 2H), 3.75 (s, 2H), 2.48-2.35 (m, 2H), 1.33 (d, J=6.2 Hz, 6H); MS (ES+) m/z 441.3 (M+1).

### Example 211

### Synthesis of N-[2-(3,3-difluoropyrrolidin-1-yl)-4-(6-methyl-2-pyridyl)-3-pyridyl]-2-isopropyl-pyrimidine-5-carboxamide

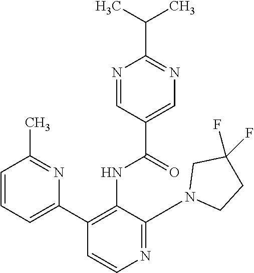 [SMILES: Cc1cccc(-c2ccnc(N3CCC(F)(F)C3)c2NC(=O)c2cnc(C(C)C)nc2)n1]

To a solution of N-[2-(3,3-difluoropyrrolidin-1-yl)-4-iodo-3-pyridyl]-2-isopropoxy-pyrimidine-5-carboxamide (0.0967 g, 0.204 mmol), tributyl-(6-methyl-2-pyridyl)stannane (0.117 g, 0.307 mmol), and 2-dicyclohexylphosphino-2′,4′,6′-triisopropylbiphenyl (0.0532 g, 0.123 mmol) in 1,4-dioxane (2.00 mL) was added palladium(II) acetate (0.0138 g, 0.0613 mmol), and the mixture was stirred at 100° C. for 20 h. After cooling to ambient temperature, the mixture was diluted with ethyl acetate (10 mL). The mixture was filtered through diatomaceous earth (i.e., Celite®) washing with ethyl acetate (20 mL) and the filtrate was concentrated in vacuo. Purification of the residue by reverse phase chromatography, eluting with a gradient of 20-50% of acetonitrile in water containing 10 mM of ammonium formate, followed by purification by preparative reverse phase HPLC, eluting with a gradient of 39-49% of acetonitrile in water containing 10 mM of ammonium formate, afforded the title compound as a yellow solid (0.0150 g, 16% yield): ¹H NMR (400 MHz; DMSO-d₆) δ 10.27 (s, 1H), 9.02 (s, 2H), 8.22 (d, J=5.0 Hz, 1H), 7.70 (t, J=7.8 Hz, 1H), 7.37 (dt, J=7.8, 0.8 Hz, 1H), 7.19 (d, J=7.7 Hz, 1H), 6.95 (d, J=5.0 Hz, 1H), 3.99-.381 (m, 2H), 3.75 (br s, 2H), 3.19 (hept, J=6.9 Hz, 1H), 2.48-2.35 (m, 5H), 1.28 (d, J=6.8 Hz, 6H); MS (ES+) m/z 439.3 (M+1).

### Example 212

### Synthesis of N-[2-(3,3-difluoropyrrolidin-1-yl)-4-(5-fluoro-2-pyridyl)-3-pyridyl]-2-isopropyl-pyrimidine-5-carboxamide

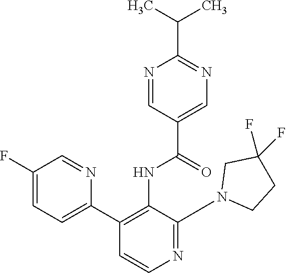 [SMILES: CC(C)c1ncc(C(=O)Nc2c(-c3ccc(F)cn3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.120 g, 0.254 mmol), tributyl-(5-fluoro-2-pyridyl)stannane (0.147 g, 0.380 mmol) and 2-dicyclohexylphosphino-2′,4′,6′-triisopropylbiphenyl (0.0660 g, 0.152 mmol) in 1,4-dioxane (2.00 mL) was added palladium(II) acetate (0.0171 g, 0.0761 mmol), and the mixture was stirred at 100° C. for 18 h. After cooling to ambient temperature, the mixture was diluted with ethyl acetate (10 mL). The mixture was filtered through diatomaceous earth (i.e., Celite®) washing with ethyl acetate (20 mL) and the filtrate was concentrated in vacuo.

Purification of the residue by reverse phase chromatography, eluting with a gradient of 20-70% of acetonitrile in water containing 10 mM of ammonium formate, followed by purification by preparative reverse phase HPLC, eluting with a gradient of 42-52% of acetonitrile in water containing 10 mM of ammonium bicarbonate, afforded the title compound as a yellow solid (0.0170 g, 15% yield): ¹H NMR (300 MHz; DMSO-d₆) δ 10.31 (s, 1H), 9.01 (s, 2H), 8.62 (d, J=2.9 Hz, 1H), 8.24 (d, J=5.0 Hz, 1H), 7.79 (td, J=8.8, 3.0 Hz, 1H), 7.65 (dd, J=8.8, 4.5 Hz, 1H), 6.96 (d, J=5.0 Hz, 1H), 3.90 (t, J=13.4 Hz, 2H), 3.75 (t, J=7.2 Hz, 2H), 3.18 (h, J=6.9 Hz, 1H), 2.41 (dt, J=14.3, 7.2 Hz, 2H), 1.29 (d, J=6.9 Hz, 6H); ¹⁰F NMR (282 MHz; DMSO-d₆) δ-101.16, -127.88; MS (ES+) m/z 443.3 (M+1).

### Example 213

### Synthesis of N-[4-(2,6-difluorophenyl)-2-(3,3-difluoropyrrolidin-1-yl)-3-pyridyl]-2-isopropyl-pyrimidine-5-carboxamide

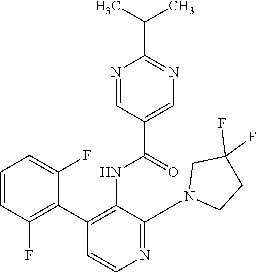 [SMILES: CC(C)c1ncc(C(=O)Nc2c(-c3c(F)cccc3F)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.0750 g, 0.158 mmol), (2,6-difluorophenyl)boronic acid (0.0500 g, 0.317 mmol) and potassium fluoride (0.0304 g, 0.523 mmol) in tetrehydrofuran (1.00 mL) and water (0.100 mL) was added mesyl[(tri-t-butylphosphine)-2-(2-aminobiphenyl)]palladium(II) (0.0136 g, 0.0238 mmol), and the mixture was stirred at 60° C. for 3 h. After cooling to ambient temperature, the mixture was diluted with ethyl acetate (5.0 mL), and the organic phase was washed with water (5.0 mL) and brine (5.0 mL). The organic phase was dried over anhydrous sodium sulfate, filtered, and concentrated in vacuo. Purification of the residue by reverse phase chromatography, eluting with a gradient of 20-70% of acetonitrile in water containing 10 mM of ammonium formate, afforded the title compound as a yellow solid (0.0283 g, 39% yield): ¹H NMR (300 MHz; DMSO-d₆) δ 10.28 (s, 1H), 8.87 (s, 2H), 8.23 (d, J=4.9 Hz, 1H), 7.41 (tt, J=8.5, 6.6 Hz, 1H), 7.13 (t, J=8.5 Hz, 2H), 6.86 (d, J=5.0 Hz, 1H), 3.89 (t, J=13.3 Hz, 2H), 3.74 (t, J=7.3 Hz, 2H), 3.16 (p, J=6.9 Hz, 1H), 2.42 (dt, J=14.2, 7.2 Hz, 2H), 1.26 (d, J=6.9 Hz, 6H); ¹⁹F NMR (282 MHz; DMSO-d₆) δ-101.06, -111.94; MS (ES+) m/z 460.7 (M+1).

### Example 214

### Synthesis of N-[2-(3,3-difluoropyrrolidin-1-yl)-4-(3,4-dihydro-2H-pyran-6-yl)-3-pyridyl]-2-isopropoxy-pyrimidine-5-carboxamide

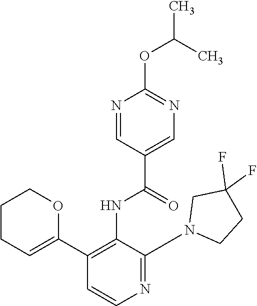 [SMILES: CC(C)Oc1ncc(C(=O)Nc2c(C3=CCCCO3)ccnc2N2CCC(F)(F)C2)cn1]

To a solution of N-[2-(3,3-difluoropyrrolidin-1-yl)-6-iodo-phenyl]-2-isopropoxy-pyrimidine-5-carboxamide (0.0750 g, 0.154 mmol), 2-(3,4-dihydro-2H-pyran-6-yl)-4,4,5,5-tetramethyl-1,3,2-dioxaborolane (0.0484 g, 0.230 mmol) and potassium carbonate (0.0743 g, 0.538 mmol) in 1,4-dioxane (1.50 mL) and water (0.500 mL) was added 1,1′-bis(diphenylphosphino)ferrocenedichloropalladium(II) (0.0251 g, 0.0307 mmol), and the mixture was stirred at 60° C. for 30 min. After cooling to ambient temperature, the mixture was diluted with ethyl acetate (5.0 mL), and the organic phase was washed with water (5.0 mL) and brine (5.0 mL). The organic phase was dried over anhydrous sodium sulfate, filtered, and concentrated in vacuo. Purification of the residue by reverse phase chromatography, eluting with a gradient of 20-100% of acetonitrile in water containing 10 mM of ammonium formate, afforded the title compound as a yellow solid (0.0346 g, 51% yield): ¹H NMR (300 MHz; DMSO-d₆), 9.87 (s, 1H), 9.07 (s, 2H), 8.08 (d, J=5.0 Hz, 1H), 6.76 (d, J=5.0 Hz, 1H), 5.30 (p, J=6.1 Hz, 1H), 5.05 (t, J=3.9 Hz, 1H), 3.94-3.77 (m, 4H), 3.70 (t, J=7.3 Hz, 2H), 2.38 (dq, J=14.2, 7.1 Hz, 2H), 2.01 (td, J=6.3, 3.8 Hz, 2H), 1.70 (p, J=6.0 Hz, 2H), 1.36 (d, J=6.2 Hz, 6H); ¹⁹F NMR (282 MHz; DMSO-d₆) δ-100.97; MS (ES+) m/z 446.0 (M+1).

### Example 215

### Synthesis of N-(2-(3,3-difluoropyrrolidin-1-yl)-4-(1H-pyrazol-5-yl)pyridin-3-yl)-6-isopropylnicotinamide

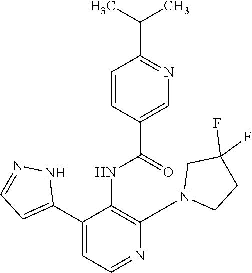 [SMILES: CC(C)c1ccc(C(=O)Nc2c(-c3ccn[nH]3)ccnc2N2CCC(F)(F)C2)cn1]

### Step 1. Preparation of 2-(3,3-difluoropyrrolidin-1-yl)-4-(1H-pyrazol-5-yl)pyridin-3-amine

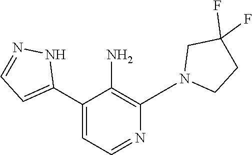 [SMILES: Nc1c(-c2ccn[nH]2)ccnc1N1CCC(F)(F)C1]

To a mixture of 2-(3,3-difluoropyrrolidin-1-yl)-4-iodopyridin-3-amine (2.00 g, 6.15 mmol), 3-(4,4,5,5-tetramethyl-1,3,2-dioxaborolan-2-yl)-1H-pyrazole (2.39 g, 12.3 mmol), potassium carbonate (2.13 g, 15.4 mmol) and [1,1′-bis(diphenylphosphino)ferrocene]dichloropalladium(II) (1.35 g, 1.85 mmol) was added dioxane (15 mL) and water (5 mL), under an atmosphere of nitrogen. The mixture was stirred at 100° C. for 2 h. After cooling to room temperature, the mixture was concentrated under reduced pressure. The residue was purified by flash silica gel chromatography, eluting with 0-30% of ethyl acetate in petroleum ether gradient to give 2-(3,3-difluoropyrrolidin-1-yl)-4-(1H-pyrazol-5-yl)pyridin-3-amine as a yellow solid (1.60 g, 97% yield): ¹H NMR (400 MHz, DMSO-d₆) J 13.16 (s, 1H), 7.88 (dd, J=1.6, 2.0 Hz, 1H), 7.56 (d, J=5.2 Hz, 1H), 7.27 (d, J=5.2 Hz, 1H), 6.87 (t, J=2.0 Hz, 1H), 6.12 (s, 2H), 3.69 (t, J=13.8 Hz, 2H), 3.46 (t, J=7.2 Hz, 2H), 2.48-2.38 (m, 2H); MS (ES+) m/z 266.1 (M+1).

### Step 2. Preparation of N-(2-(3,3-difluoropyrrolidin-1-yl)-4-(1H-pyrazol-5-yl)pyridin-3-yl)-6-isopropylnicotinamide

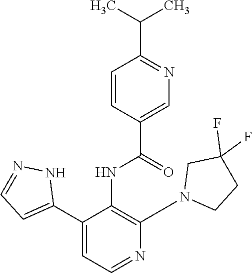 [SMILES: CC(C)c1ccc(C(=O)Nc2c(-c3ccn[nH]3)ccnc2N2CCC(F)(F)C2)cn1]

To a mixture of 2-(3,3-difluoropyrrolidin-1-yl)-4-(1H-pyrazol-5-yl)pyridin-3-amine (0.0700 g, 0.264 mmol), 6-isopropylnicotinic acid (0.0880 g, 0.533 mmol), 2,4,6-tripropyl-1,3,5,2,4,6-trioxatriphosphinane2,4,6-trioxide (0.252 g, 0.396 mmol, 50% in ethyl acetate) in tetrahydrofuran (5 mL) was added N-ethyl-N-isopropylpropan-2-amine (0.103 g, 0.797 mmol) and the mixture was stirred at 25° C. for 1 h. Then the mixture was stirred at 70° C. for 12 h. After cooling to ambient temperature, the mixture was concentrated under reduced pressure. The residue was purified by flash silica gel chromatography, eluting with 0-20% of ethyl acetate in petroleum ether to afforded the title compound as a colorless solid (0.0161 g, 14% yield): ¹H NMR (400 MHz, CDCl₃) δ 9.35 (d, J=1.8 Hz, 1H), 8.57 (d, J=2.8 Hz, 1H), 8.47-8.39 (m, 1H), 7.86 (d, J=5.4 Hz, 1H), 7.43 (d, J=8.4 Hz, 1H), 7.31 (d, J=4.8 Hz, 1H), 7.28-7.27 (m, 1H), 7.05 (d, J=2.8 Hz, 1H), 5.78 (s, 1H), 4.00-3.53 (m, 4H), 3.36-3.14 (m, 1H), 2.62-2.41 (m, 2H), 1.42 (s, 3H), 1.40 (s, 3H); MS (ES+) m/z 413.4 (M+1).

### Example 216-221

In a similar manner as described in Example 215, utilizing the appropriately substituted starting materials and intermediates, the following compounds were

| Example | Structure | | MS (ES+) | |
|---|---|---|---|---|
| No. | Name | Yield | m/z | ¹H-NMR |
| 216 | 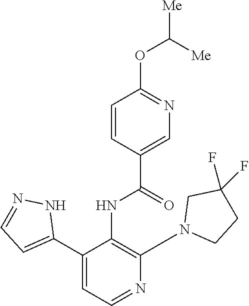 [SMILES: CC(C)Oc1ccc(C(=O)Nc2c(-c3ccn[nH]3)ccnc2N2CCC(F)(F)C2)cn1] N-(2-(3,3-difluoropyrrolidin- 1-yl)-4-(1H-pyrazol-5- yl)pyridin-3-yl)-6- isopropoxynicotinamide | 3% | 429.0 (M + 1) | (400 MHz; CDCl₃): δ 9.06 (d, J = 2.5 Hz, 1H), 8.52 (d, J = 3.0 Hz, 1H), 8.38 (dd, J = 8.8, 2.5 Hz, 1H), 7.80 (d, J = 5.3 Hz, 1H), 7.24 (d, J = 5.3 Hz, 1H), 6.99 (d, J = 3.0 Hz, 1H), 6.82 (d, J = 8.8 Hz, 1H), 5.66 (s, 2H), 5.49 (dt, J = 12.4, 6.2 Hz, 1H), 3.69 (t, J = 13.1 Hz, 2H), 3.54 (t, J = 7.1 Hz, 2H), 2.47 (tt, J = 14.4, 7.2 Hz, 2H), 1.43 (d, J = 6.2 Hz, 7H). |
| 217 | 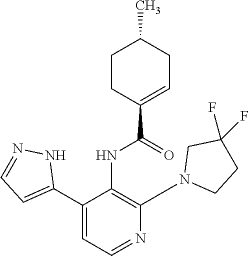 (1r,4r)-N-(2-(3,3-difluoropyrrolidin- 1-yl)-4-(1H-pyrazol-5- yl)pyridin-3-yl)-4-methylcyclohexane-1- carboxamide | 18% | 405.2 (M + 1) | (400 MHz; DMSO- d₆) δ 8.09 (t, J = 4.2 Hz, 1H), 7.59- 7.55 (m, 1H), 7.46- 7.38 (m, 2H), 7.35- 7.19 (m, 4H), 6.72- 6.70 (m, 1H), 4.02- 3.88 (m, 2H), 3.85- 3.66 (m, 3H), 3.18 (s, 3H), 3.14-2.95 (m, 4H), 2.50-2.37 (m, 2H), 1.72-1.55 (m, 4H) |
| 218 | 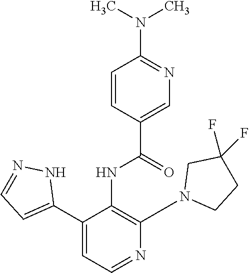 [SMILES: CN(C)c1ccc(C(=O)Nc2c(-c3ccn[nH]3)ccnc2N2CCC(F)(F)C2)cn1] N-(2-(3,3-difluoropyrrolidin- 1-yl)-4-(1H-pyrazol-5- yl)pyridin-3-yl)-6-(dimethylamino) nicotinamide formate salt | 23% | 414.0 (M + 1) | (400 MHz; DMSO- d₆) δ 13.37-12.90 (m, 1H), 9.77 (s, 1H), 8.73 (s, 1H), 8.42 (s, 1H), 8.16- 8.07 (m, 1H), 8.06-7.98 (m, 1H), 7.77-7.64 (m, 1H), 7.20 (d, J = 3.4 Hz, 1H), 6.70 (d, J = 9.0 Hz, 1H), 6.55 (s, 1H), 3.98- 3.66 (m, 4H), 3.11 (s, 6H), 2.43-2.35 (m, 2H) |
| 219 | 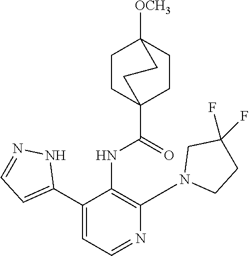 [SMILES: COC12CCC(C(=O)Nc3c(-c4ccn[nH]4)ccnc3N3CCC(F)(F)C3)(CC1)CC2] N-(2-(3,3-difluoropyrrolidin- 1-yl)-4-(1H-pyrazol-5- yl)pyridin-3-yl)-4-methoxybicyclo [2.2.2]octane-1-carboxamide | 41% | 432.2 (M + 1) | (400 MHz, MeOD- d₄) δ8.07 (d, J = 3.2 Hz, 1H), 7.73 (s, 1H), 7.02 (d, J = 1.2 Hz, 1H), 6.60 (s, 1H), 3.88- 3.68 (m, 4H), 3.18 (s, 3H), 2.39 (td, J = 6.8, 13.6 Hz, 2H), 1.91 (d, J = 5.2 Hz, 6H), 1.79- 1.66 (m, 6H) |
| 220 | 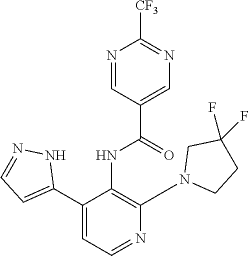 [SMILES: O=C(Nc1c(-c2ccn[nH]2)ccnc1N1CCC(F)(F)C1)c1cnc(C(F)(F)F)nc1] N-(2-(3,3-difluoropyrrolidin- 1-yl)-4-(1H-pyrazol-5- yl)pyridin-3-yl)-2-(trifluoromethyl) pyrimidine-5-carboxamide | 28% | 440.2 (M + 1) | (400 MHz, DMSO- d₆) δ 13.13 (s, 1H), 10.56 (s, 1H), 9.52 (s, 2H), 8.16 (d, J = 5.2 Hz, 1H), 7.75 (s, 1H), 7.19 (d, J = 4.4 Hz, 1H), 6.70 (s, 1H), 3.96-3.84 (m, 2H), 3.82-3.67 (m, 2H), 2.44-2.35 (m, 2H) |
| 221 | 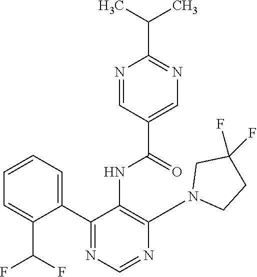 [SMILES: CC(C)c1ncc(C(=O)Nc2c(-c3ccccc3C(F)F)ncnc2N2CCC(F)(F)C2)cn1] N-(4-(2-(difluoromethyl) phenyl)-6-(3,3-difluoropyrrolidin- 1-yl)pyrimidin-5-yl)-2- isopropylpyrimidine-5-carboxamide | 77% | 475.2 (M + 1) | (400 MHz, CDCl₃) δ 8.69-8.61 (m, 3H), 7.74 (d, J = 7.2 Hz, 1H), 7.54- 7.43 (m, 2H), 7.32 (d, J = 7.2 Hz, 1H), 7.16 (s, 1H), 6.85-6.52 (t, 1H), 4.08-3.91 (m, 4H), 3.25 (td, J = 7.0, 13.8 Hz, 1H), 2.49-2.35 (m, 2H), 1.33 (d, J = 7.0 Hz, 6H) |

### Example 222

### Synthesis of N-(2-(3,3-difluoropyrrolidin-1-yl)-4-(1H-pyrazol-5-yl)pyridin-3-yl)isoindoline-2-carboxamide

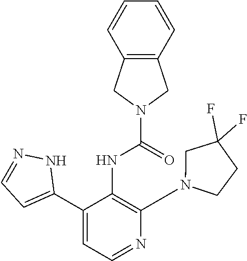 [SMILES: O=C(Nc1c(-c2ccn[nH]2)ccnc1N1CCC(F)(F)C1)N1Cc2ccccc2C1]

### Step 1. Preparation of N-(2-(3,3-difluoropyrrolidin-1-yl)-4-iodopyridin-3-yl)isoindoline-2-carboxamide

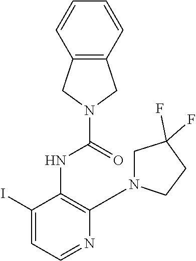 [SMILES: O=C(Nc1c(I)ccnc1N1CCC(F)(F)C1)N1Cc2ccccc2C1]

A mixture of tert-butyl (2-(3,3-difluoropyrrolidin-1-yl)-4-iodopyridin-3-yl)carbamate (0.200 g, 0.470 mmol), isoindoline (0.0561 g, 0.470 mmol), 4-dimethylaminopyridin (0.0632 g, 0.517 mmol) and 4A molecular sieve (0.300 g) in dimethylformamide (2 mL) was stirred at 25° C. for 1 h under an atmosphere of nitrogen. The reaction mixture was stirred at 80° C. for 12 h and 110° C. for 12 h. The reaction mixture was cooled to ambient temperature. The mixture was purified by reversed-phase column chromatography, eluting with 0.1% formic acid in water to afford the title compound as a gray solid (0.0800 g, 36% yield); ¹H NMR (400 MHz, DMSO-d₆) δ 8.20 (s, 1H), 7.67 (d, J=5.0 Hz, 1H), 7.40-7.32 (m, 4H), 7.25 (d, J=5.0 Hz, 1H), 4.86 (s, 4H), 4.10-3.97 (m, 1H), 3.92-3.77 (m, 2H), 3.75-3.60 (m, 1H), 2.46-2.67 (m, 2H).

### Step 2. Preparation of N-(2-(3,3-difluoropyrrolidin-1-yl)-4-(1H-pyrazol-5-yl)pyridin-3-yl)isoindoline-2-carboxamide

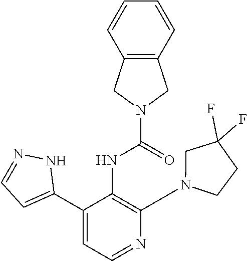 [SMILES: O=C(Nc1c(-c2ccn[nH]2)ccnc1N1CCC(F)(F)C1)N1Cc2ccccc2C1]

To a mixture of N-(2-(3,3-difluoropyrrolidin-1-yl)-4-iodopyridin-3-yl)isoindoline-2-carboxamide (0.0400 g, 0.0851 mmol), 5-(4,4,5,5-tetramethyl-1,3,2-dioxaborolan-2-yl)-1H-pyrazole (0.0247 g, 0.127 mmol), potassium carbonate (0.0352 g, 0.255 mmol) and [1,1-bis(diphenylphosphino)ferrocene]dichloro-palladium(II) (0.00622 g, 0.00851 mmol) was added dioxane (2 mL) and water (0.4 mL). The mixture was stirred at 60° C. for 2 h under nitrogen. The reaction mixture was cooled to ambient temperature and concentrated under reduced pressure. The residue was purified by prep-HPLC, eluting with 18-48% of acetonitrile in water containing formic acid (0.1%) to afford the title compound as a colorless solid (0.0183 g, 33% yield): ¹H NMR (400 MHz, DMSO-d₆) δ 13.14 (br s, 1H), 8.31 (s, 1H), 8.04 (d, J=3.4 Hz, 1H), 7.77 (s, 1H), 7.40-7.27 (m, 4H), 7.16 (s, 1H), 6.66 (d, J=0.8 Hz, 1H), 4.73 (s, 4H), 4.03-3.88 (m, 2H), 3.78 (s, 2H), 2.45-2.35 (m, 2H); MS (ES+) m/z 411.2 (M+1).

### Example 223

### Synthesis of N-(4-(3,3-difluoropyrrolidin-1-yl)-6-(1H-pyrazol-5-yl)pyrimidin-5-yl)-6-isopropylnicotinamide

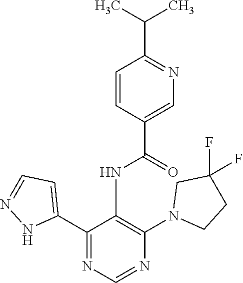 [SMILES: CC(C)c1ccc(C(=O)Nc2c(-c3ccn[nH]3)ncnc2N2CCC(F)(F)C2)cn1]

### Step 1. Preparation of 4-(3,3-difluoropyrrolidin-1-yl)-5-nitro-6-(1H-pyrazol-5-yl)pyrimidine

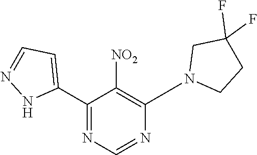 [SMILES: O=[N+]([O-])c1c(-c2ccn[nH]2)ncnc1N1CCC(F)(F)C1]

To a mixture of 4-chloro-6-(3,3-difluoropyrrolidin-1-yl)-5-nitropyrimidine (1.00 g, 3.78 mmol), 5-(4,4,5,5-tetramethyl-1,3,2-dioxaborolan-2-yl)-1H-pyrazole (0.807 g, 4.16 mmol) and potassium carbonate (1.57 g, 11.3 mmol) in dioxane (16 mL) and water (2 mL) was added [1,1′-bis(diphenylphosphino)ferrocene]dichloropalladium(II) (0.277 g, 0.378 mmol). The mixture was stirred at 90° C. under nitrogen atmosphere for 12 h.

After cooling to ambient temperature, the mixture was poured into water (20 mL) and extracted with ethyl acetate (3×20 mL). The combined organic extracts were dried over anhydrous sodium sulfate, filtered and concentrated in vacuo. The residue was purified by column chromatography, eluting with 17% ethyl acetate in petroleum ether to afford the title compound as a black-brown solid (0.500 g, 44% yield): ¹H NMR (400 MHz, DMSO-d₆) δ 8.66-8.60 (t, 2H), 7.90 (s, J=1.2 Hz, 1H), 6.66 (dd, J=1.6, 2.8 Hz, 1H), 3.96 (t, J=12.8 Hz, 2H), 3.79 (t, 2H), 2.62-2.52 (m, 2H); MS (ES+) m/z 297.0 (M+1).

### Step 2. Preparation of 4-(3,3-difluoropyrrolidin-1-yl)-6-(1H-pyrazol-5-yl)pyrimidin-5-amine

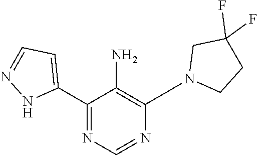 [SMILES: Nc1c(-c2ccn[nH]2)ncnc1N1CCC(F)(F)C1]

To a mixture of 4-(3,3-difluoropyrrolidin-1-yl)-5-nitro-6-(1H-pyrazol-5-yl)pyrimidine (0.500 g, 1.69 mmol) in methanol (8 mL) was added palladium on carbon (0.200 g, 1.69 mmol, 10% purity) in one portion. The mixture was stirred at 25° C. under hydrogen atmosphere for 12 h. The resulting mixture was filtered over diatomaceous earth (i.e., Celite®) and concentrated under reduced pressure to afford 4-(3,3-difluoropyrrolidin-1-yl)-6-(1H-pyrazol-5-yl)pyrimidin-5-amine as a colorless oil (0.450 g, 74% yield): ¹H NMR (400 MHz, DMSO-d₆) δ 8.64 (dd, J=0.8, 2.8 Hz, 1H), 8.06 (s, 1H), 7.90 (d, J=0.8, 1.6 Hz, 1H), 6.60 (dd, J=1.8, 2.8 Hz, 1H), 5.93 (s, 2H), 4.06-3.99 (m, 2H), 3.86-3.82 (m, 2H), 2.48-2.36 (m, 2H); MS (ES+) m/z 267.1 (M+1).

### Step 3. Preparation of N-(4-(3,3-difluoropyrrolidin-1-yl)-6-(1H-pyrazol-5-yl)pyrimidin-5-yl)-6-isopropylnicotinamide

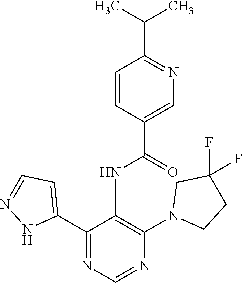 [SMILES: CC(C)c1ccc(C(=O)Nc2c(-c3ccn[nH]3)ncnc2N2CCC(F)(F)C2)cn1]

To a mixture of 4-(3,3-difluoropyrrolidin-1-yl)-6-(1H-pyrazol-5-yl)pyrimidin-5-amine (0.100 g, 0.376 mmol), 6-isopropylnicotinic acid (0.0744 g, 0.450 mmol) and N,N-diisopropylethylamine (0.145 g, 1.13 mmol) in tetrahydrofuran (2 mL) was added 2-chloro-1-methyl-pyridin-1-ium iodide (0.115 g, 0.451 mmol). The mixture was stirred at 60° C. for 12 h. After cooling to ambient temperature, the reaction mixture was concentrated under reduced pressure. The residue was poured into water (20 mL) and extracted with ethyl acetate (3×15 mL). The combined organic extracts were dried over anhydrous sodium sulfate, filtered and concentrated in vacuo. The residue was purified by prep-HPLC eluting with a gradient of 35-65% of acetonitrile in water containing 0.225% formic acid, to afford the title compound as a colorless solid (0.0177 g, 11% yield): ¹H NMR (400 MHz, CDCl₃) δ 10.91 (s, 1H), 9.16 (d, J=2.0 Hz, 1H), 8.55 (d, J=2.8 Hz, 1H), 8.39 (s, 1H), 8.22 (dd, J=2.0, 8.0 Hz, 1H), 7.83 (d, J=0.8 Hz, 1H), 7.35 (d, J=8.2 Hz, 1H), 6.46 (t, J=2.0 Hz, 1H), 4.04-3.83 (m, 4H), 3.19 (m, J=6.8, 13.8 Hz, 1H), 2.52-2.34 (m, 2H), 1.37 (d, J=6.8 Hz, 6H); MS (ES+) m/z 414.1 (M+1).

### Example 224

### Synthesis of N-(2-(3,3-difluoropyrrolidin-1-yl)-4-(1H-pyrazol-5-yl)pyridin-3-yl)-4-(2-(trifluoromethyl)oxetan-2-yl)benzamide formate salt

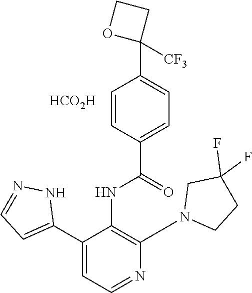 [SMILES: O=C(Nc1c(-c2ccn[nH]2)ccnc1N1CCC(F)(F)C1)c1ccc(C2(C(F)(F)F)CCO2)cc1 ; "HCO2H"]

### Step 1. Preparation of N-(2-(3,3-difluoropyrrolidin-1-yl)-4-iodopyridin-3-yl)-4-(2,2,2-trifluoroacetyl)benzamide

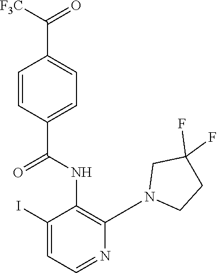 [SMILES: O=C(Nc1c(I)ccnc1N1CCC(F)(F)C1)c1ccc(C(=O)C(F)(F)F)cc1]

To a mixture of 4-(2,2,2-trifluoroacetyl)benzoic acid (0.250 g, 1.15 mmol), N,N-diisopropylethylamine (0.741 g, 5.73 mmol) and 2-chloro-1-methyl-pyridin-1-ium iodide (0.381 g, 1.49 mmol) in tetrahydrofuran (5 mL) was added 2-(3,3-difluoropyrrolidin-1-yl)-4-iodo-pyridin-3-amine (0.373 g, 1.15 mmol) in one portion at 20° C. The mixture was stirred at 65° C. for 12 h. The mixture was cooled to 20° C. and evaporated under reduced pressure. The mixture was poured into saturated aqueous sodium bicarbonate (10 mL) and extracted with ethyl acetate (3×10 mL). The combined organic layers were washed with brine (10 mL), dried over anhydrous sodium sulfate, filtered and the filtrate was evaporated under reduced pressure. The combined residue was purified by reversed phase column, eluting with 0.1% formic acid in water to afford the title compound as a light yellow solid (0.350 g, 58% yield): ¹H NMR (400 MHz, CDCl₃) δ 8.25 (d, J=8.0 Hz, 2H), 8.13 (d, J=8.0 Hz, 2H), 7.79 (d, J=5.2 Hz, 1H), 7.47 (br s, 1H), 7.30 (d, J=5.2 Hz, 1H), 3.92-3.72 (m, 4H), 2.44-2.29 (m, 2H).

### Step 2. Preparation of N-(2-(3,3-difluoropyrrolidin-1-yl)-4-iodo-pyridin-3-yl)-4-(2-(trifluoromethyl)oxetan-2-yl)benzamide

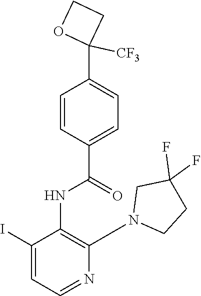 [SMILES: O=C(Nc1c(I)ccnc1N1CCC(F)(F)C1)c1ccc(C2(C(F)(F)F)CCO2)cc1]

To a mixture of potassium 2-methylpropan-2-olate (0.224 g, 2.00 mmol) in dimethylsulfoxide (6 mL) was added trimethylsulfonium iodide (0.408 g, 2.00 mmol) in one portion at 20 0° C. The mixture was stirred at 20° C. for 10 min then a solution of N-(2-(3,3-difluoropyrrolidin-1-yl)-4-iodopyridin-3-yl)-4-(2,2,2-trifluoro-acetyl)benzamide (0.300 g, 0.571 mmol) in dimethylsulfoxide (1 mL) was added. The mixture was stirred at 20° C. for 15 h. The mixture was quenched with methanol (1 mL). The residue was purified by reversed phase column, eluting with 0.1 formic acid to afford the title compound as a colorless solid (0.200 g, 63% yield): ¹H NMR (400 MHz, CDCl₃) δ 8.01 (d, J=8.0 Hz, 2H), 7.76 (d, J=5.2 Hz, 1H), 7.60 (d, J=8.0 Hz, 3H), 7.29-7.26 (m, 1H), 4.97-4.81 (m, 1H), 4.72-4.57 (m, 1H), 3.97-3.71 (m, 4H), 3.39-3.28 (m, 1H), 3.01-2.89 (m, 1H), 2.44-2.28 (m, 2H).

### Step 3. Preparation of N-(2-(3,3-difluoropyrrolidin-1-yl)-4-(1H-pyrazol-5-yl)pyridin-3-yl)-4-(2-(trifluoromethyl)oxetan-2-yl)benzamide formate salt

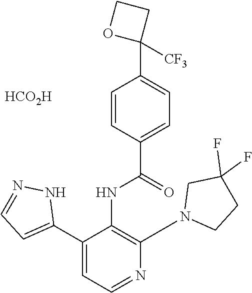 [SMILES: "HCO2H" ; O=C(Nc1c(-c2ccn[nH]2)ccnc1N1CCC(F)(F)C1)c1ccc(C2(C(F)(F)F)CCO2)cc1]

To a mixture of N-(2-(3,3-difluoropyrrolidin-1-yl)-4-iodopyridin-3-yl)i-534C (trifluoromethyl)oxetan-2-yl)-benzamide (0.200 g, 0.361 mmol) and 3-(4,4,5,5-tetramethyl-1,3,2-dioxaborolan-2-yl)-1H-pyrazole (0.140 g, 0.723 mmol) in dioxane (6 mL) and water (2 mL) were added [1,1′-bis(diphenylphosphino)ferrocene]-dichloropalladium(II) (0.0794 g, 0.108 mmol) and potassium carbonate (0.0999 g, 0.723 mmol) in one portion at 20 0C. The mixture was stirred at 100° C. under nitrogen atmosphere for 1 h. The mixture was cooled to 20° C. and poured into water (10 mL). The mixture was extracted with ethyl acetate (3×10 mL). The combined organic layers were washed with brine (10 mL), dried over anhydrous sodium sulfate, filtered and the filtrate was evaporated under reduced pressure. The residue was purified by prep-HPLC, eluting with a gradient of 25-55% acetonitrile in water containing 0.225% formic acid, to afford the title compound as a colorless solid (0.0223 g, 70% yield, 95%): ¹H NMR (400 MHz, DMSO-d₆) δ 13.22-13.05 (m, 1H), 10.13 (s, 1H), 8.32 (s, 1H), 8.13 (d, J=4.0 Hz, 1H), 8.07 (d, J=8.0 Hz, 2H), 7.75 (d, J=1.6 Hz, 1H), 7.57 (d, J=8.4 Hz, 2H), 7.20 (d, J=1.6 Hz, 1H), 6.59 (br s, 1H), 4.84-4.71 (m, 1H), 4.67-4.52 (m, 1H), 4.00-3.62 (m, 4H), 3.27-3.23 (m, 1H), 3.10-2.99 (m, 1H), 2.45-2.34 (m, 2H); MS (ES+) m/z 494.2 (M+1).

### Example 225

In a similar manner as described in Example 224, utilizing the appropriately substituted starting materials and intermediates, the following compounds were

| Example | Structure | | MS (ES+) | |
|---|---|---|---|---|
| No. | Name | Yield | m/z | ¹H-NMR |
| 225 | 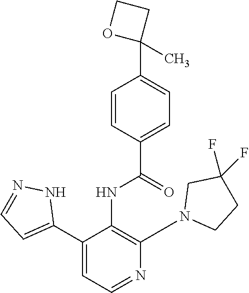 [SMILES: CC1(c2ccc(C(=O)Nc3c(-c4ccn[nH]4)ccnc3N3CCC(F)(F)C3)cc2)CCO1] N-(2-(3,3-difluoropyrrolidin-1-yl)-4- (1H-pyrazol-5-yl)pyridin-3-yl)-4-(2- methyloxetan-2-yl)benzamide | 11% | 440.2 (M + 1) | (400 MHz, DMSO-d₆) δ 13.36-12.86 (m, 1H), 10.22-9.79 (m, 1H), 8.17-8.07 (m, 1H), 7.99 (d, J = 8.2 Hz, 2H), 7.70 (s, 1H), 7.52 (d, J = 8.4 Hz, 2H), 7.19 (d, J = 4.8 Hz, 1H), 6.57 (d, J = 2.2 Hz, 1H), 4.55 (td, J = 6.4, 8.4 Hz, 1H), 4.40 (td, J = 6.4, 8.8 Hz, 1H), 3.94- 3.68 (m, 4H), 2.82 (ddd, J = 6.8, 8.8, 10.8 Hz, 1H), 2.72-2.66 (m, 1H), 2.44-2.32 (m, 2H), 1.68 (s, 3H). |

### Example 226

### Synthesis of N-(2-(3,3-difluoropyrrolidin-1-yl)-4-(2-fluorophenyl)pyridin-3-yl)-5-isopropylpyrazine-2-carboxamide

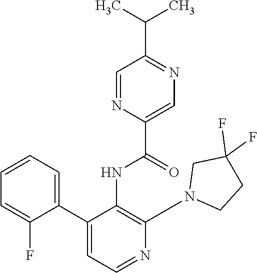 [SMILES: CC(C)c1cnc(C(=O)Nc2c(-c3ccccc3F)ccnc2N2CCC(F)(F)C2)cn1]

### Step 1. Preparation of methyl 5-(prop-1-en-2-yl)pyrazine-2-carboxylate

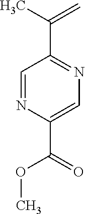

To a mixture of methyl 5-chloropyrazine-2-carboxylate (5.00 g, 28.9 mmol), 2-isopropenyl-4,4,5,5-tetramethyl-1,3,2-dioxaborolane (7.30 g, 43.5 mmol), cesium carbonate (28.3 g, 86.9 mmol) and [1,1′-bis(diphenyl-phosphino)ferrocene]dichloropalladium(II) (2.12 g, 2.90 mmol) was added dioxane (50 mL) and water (10 mL). The mixture was stirred at 80° C. for 12 h under a nitrogen atmosphere. The reaction mixture was cooled to ambient temperature, filtered and concentrated under reduced pressure. The residue was purified by flash silica gel chromatography, eluting with a gradient of 0-20% of ethyl acetate in petroleum ether to afford the title compound as a colorless solid (2.50 g, 48% yield): ¹H NMR (400 MHz, CDCl₃) δ 9.24 (d, J=1.4 Hz, 1H), 8.88 (d, J=1.4 Hz, 1H), 6.08 (s, 1H), 5.61-5.49 (m, 1H), 4.04 (s, 3H), 2.26 (s, 3H).

### Step 2. Preparation of methyl 5-isopropylpyrazine-2-carboxylate

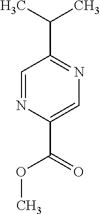

To a solution of methyl 5-(prop-1-en-2-yl)pyrazine-2-carboxylate (1.00 g, 5.61 mmol) and 2-nitrobenzenesulfonyl chloride (2.49 g, 11.2 mmol) in acetonitrile (60 ml) was added hydrazine monohydrate (1.13 g, 22.6 mmol) at 0° C. under nitrogen, then the mixture was stirred at 25° C. for 48 h. To the mixture was added water (50 mL), then the mixture was extracted with ethyl acetate (3×50 mL). The combined organic layers were washed with brine (3×50 mL), dried over sodium sulfate, filtered and concentrated under reduced pressure. The residue was purified by flash silica gel chromatography, eluting with a gradient of 0-25% of ethyl acetate in petroleum ether, followed by preparative HPLC, eluting with a gradient of 15-45% of acetonitrile in water containing 0.1% trifluoroacetic acid. The desired fraction was collected and extracted with ethyl acetate (3×50 mL). The combined organic layers were washed with brine (100 mL), dried over sodium sulfate, filtered and concentrated under reduced pressure to afford the title compound as a yellow oil (0.600 g, 59%): ¹H NMR (400 MHz, CDCl₃) δ 9.22 (d, J=1.4 Hz, 1H), 8.59 (d, J=1.4 Hz, 1H), 4.03 (s, 3H), 3.21 (td, J=6.8, 13.8 Hz, 1H), 1.37 (d, J=6.8 Hz, 6H).

### Step 3. Preparation of 5-isopropylpyrazine-2-carboxylic acid

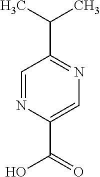

To a mixture of methyl 5-isopropylpyrazine-2-carboxylate (0.100 g, 0.555 mmol) and lithium hydroxide (0.0466 g, 1.11 mmol) was added tetrahydrofuran (5 mL) and water (5 mL). The mixture was stirred at 25° C. for 12 h. The reaction mixture was concentrated under reduced pressure to remove tetrahydrofuran. The residue was diluted with water (10 mL). The pH was adjusted to 2 with 1 N hydrochloric acid. The mixture was extracted with dichloromethane (3×10 mL). The combined organic layers were washed with brine (30 mL), dried over sodium sulfate, filtered and concentrated under reduced pressure to afford the title compound as a colorless solid (0.0600 g, crude):¹H NMR (400 MHz, DMSO-d₆) δ 13.56 (s, 1H), 9.10 (d, J=1.2 Hz, 1H), 8.73 (d, J=1.2 Hz, 1H), 3.21 (spt, J=6.8 Hz, 1H), 1.28 (d, J=6.8 Hz, 6H).

### Step 4. Preparation of N-(2-(3,3-difluoropyrrolidin-1-yl)-4-(2-fluorophenyl)pyridin-3-yl)-5-isopropylpyrazine-2-carboxamide

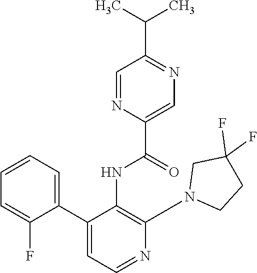 [SMILES: CC(C)c1cnc(C(=O)Nc2c(-c3ccccc3F)ccnc2N2CCC(F)(F)C2)cn1]

To a mixture of 5-isopropylpyrazine-2-carboxylic acid (0.0600 g, 0.361 mmol), 2-(3,3-difluoropyrrolidin-1-yl)-4-(2-fluorophenyl)pyridin-3-amine (0.105 g, 0.361 mmol), 2-chloro-1-methyl-pyridin-1-ium iodide (0.110 g, 0.433 mmol) and N,N-diisopropylethylamine (0.233 g, 1.81 mmol) in tetrahydrofuran (5 mL) was stirred at 65° C. for 12 h. The reaction mixture was cooled to ambient temperature and concentrated under reduced pressure. The residue was purified by preparative HPLC, eluting with a gradient of 45-75% of acetonitrile in water containing 0.225% formic acid to afford the title compound as a light yellow solid (0.0832 g, 52% yield): ¹H NMR (400 MHz, MeOD-d₄) δ 8.94 (d, J=1.4 Hz, 1H), 8.57 (d, J=1.4 Hz, 1H), 8.20 (d, J=5.0 Hz, 1H), 7.39-7.32 (m, 1H), 7.32-7.27 (m, 1H), 7.12 (dt, J=1.0, 7.6 Hz, 1H), 7.07 (ddd, J=1.0, 8.6, 10.0 Hz, 1H), 6.82 (d, J=5.0 Hz, 1H), 3.89 (t, J=13.2 Hz, 2H), 3.81 (t, J=7.0 Hz, 2H), 3.21 (td, J=6.8, 13.8 Hz, 1H), 2.46-2.27 (m, 2H), 1.34 (d, J=7.0 Hz, 6H); MS (ES+) m/z 442.0 (M+1).

### Example 227

### Synthesis of N-(2-(3,3-difluoropyrrolidin-1-yl)-4-(2-fluorophenyl)pyridin-3-yl)-5-isopropoxypyrazine-2-carboxamide

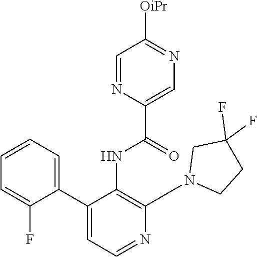 [SMILES: CC(C)Oc1cnc(C(=O)Nc2c(-c3ccccc3F)ccnc2N2CCC(F)(F)C2)cn1]

### Step 1. Preparation of 5-chloro-N-(2-(3,3-difluoropyrrolidin-1-yl) -4-(2-fluorophenyl)pyridin-3-yl)pyrazine-2-carboxamide

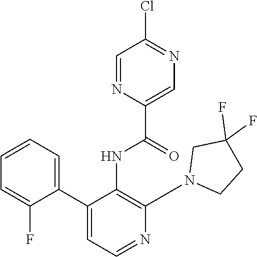 [SMILES: O=C(Nc1c(-c2ccccc2F)ccnc1N1CCC(F)(F)C1)c1cnc(Cl)cn1]

To a solution of 5-chloropyrazine-2-carboxylic acid (0.108 g, 0.682 mmol), N-ethyl-N-isopropylpropan-2-amine (0.132 g, 1.02 mmol, 0.178 mL) and 2-chloro-1-methylpyridinium iodide (0.131 g, 0.511 mmol) was added tetrahydrofuran (3 mL). The mixture was stirred at 20° C. for 10 min, then 2-(3,3-difluoropyrrolidin-1-yl)-4-(2-fluorophenyl)pyridin-3-amine (0.100 g, 0.341 mmol) was added. The resulting mixture was stirred at 65° C. for 12 h. The reaction mixture was filtered over diatomaceous earth (i.e., Celite®). The filtrate was diluted with ethyl acetate (20 mL) and water (20 mL). The aqueous phase was extracted with ethyl acetate (3×10 mL). The combined extracts were washed with brine (20 mL), dried over sodium sulfate, and filtered. The filtrate was concentrated under reduced pressure. The residue was purified by flash silica gel chromatography, eluting with a gradient of 55-65% of ethyl acetate in petroleum ether, to afford the title compound as a colorless solid (0.096 g, 0.221 mmol, 65% yield): ¹H NMR (400 MHz, CDCl₃) δ 9.05-8.97 (m, 2H), 8.62-8.47 (m, 1H), 8.29 (d, J=5.2 Hz, 1H), 7.38-7.28 (m, 2H), 7.22-7.12 (m, 1H), 7.03 (t, J=9.4 Hz, 1H), 6.81 (d, J=5.2 Hz, 1H), 3.94-3.87 (m, 4H), 2.42-2.35 (m, 1H).

### Step 2. Preparation of N-(2-(3,3-difluoropyrrolidin-1-yl)-4-(2-fluorophenyl)pyridin-3-yl)-5-isopropoxypyrazine-2-carboxamide

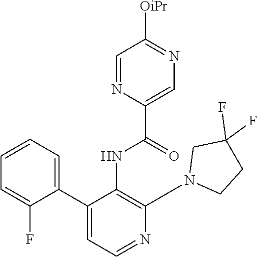 [SMILES: CC(C)Oc1cnc(C(=O)Nc2c(-c3ccccc3F)ccnc2N2CCC(F)(F)C2)cn1]

To a solution of 5-chloro-N-(2-(3,3-difluoropyrrolidin-1-yl)-4-(2-fluorophenyl)pyridin-3-yl)pyrazine-2-carboxamide (0.0960 g, 0.221 mmol) in isopropanol (5 mL) was added cesium carbonate (0.216 g, 0.664 mmol). The mixture was stirred at 80° C. for 4 h. The reaction mixture was cooled to ambient temperature and filtered over diatomaceous earth (i.e., Celite®). The filtrate was concentrated under reduced pressure. The residue was purified by preparative HPLC, eluting with a gradient of 55-75% of acetonitrile in water containing 0.225% formic acid, to afford the title compound as a colorless solid (0.0343 g, 33% yield): ¹H NMR (400 MHz, DMSO-d₆) δ 10.00 (s, 1H), 8.60 (d, J=1.2 Hz, 1H), 8.24 (d, J=1.2 Hz, 1H), 8.17 (d, J=5.2 Hz, 1H), 7.43-7.27 (m, 2H), 7.20 (d, J=9.3 Hz, 1H), 7.10 (dt, J=0.9, 7.5 Hz, 1H), 6.78 (dd, J=0.9, 5.0 Hz, 1H), 5.36-5.22 (m, 1H), 3.89 (t, J=13.6 Hz, 2H), 3.73 (t, J=7.2 Hz, 2H), 2.38 (td, J=7.2, 14.2 Hz, 2H), 1.33 (d, J=6.0 Hz, 6H); MS (ES+) m/z 458.2 (M+1).

### Example 228-235

In a similar manner as described in Example 227 step 1, utilizing the appropriately substituted starting materials and intermediates, the following compounds were prepared:

| | | | MS | |
|---|---|---|---|---|
| Example | Structure | | (ES+) | |
| No. | Name | Yield | m/z | ¹H-NMR |
| 228 | 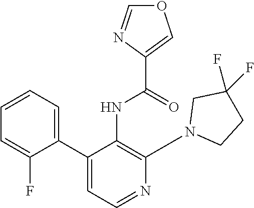 [SMILES: O=C(Nc1c(-c2ccccc2F)ccnc1N1CCC(F)(F)C1)c1cocn1] N-(2-(3,3-difluoropyrrolidin-1- yl)-4-(2-fluorophenyl)pyridin- 3-yl)oxazole-4-carboxamide | 24% | 389.0 (M + 1) | (400 MHz, DMSO-d₆) δ 9.73 (s, 1H), 8.59 (d, J = 0.8 Hz, 1H), 8.49 (d, J = 0.8 Hz, 1H), 8.16 (d, J = 5.0 Hz, 1H), 7.35 (q, J = 7.2 Hz, 2H), 7.26-7.18 (m, 1H), 7.17-7.10 (m, 1H), 6.77 (d, J = 4.4 Hz, 1H), 3.90 (t, J = 13.6 Hz, 2H), 3.74 (t, J = 7.2 Hz, 2H), 2.47-2.31 (m, 2H). |
| 229 | 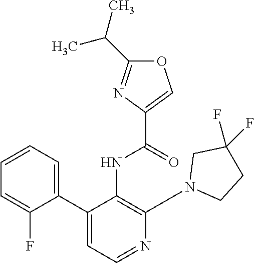 [SMILES: CC(C)c1nc(C(=O)Nc2c(-c3ccccc3F)ccnc2N2CCC(F)(F)C2)co1] N-(2-(3,3-difluoropyrrolidin-1- yl)-4-(2-fluorophenyl)pyridin- 3-yl)-2-isopropyloxazole-4- carboxamide | 5% | 430.1 (M + 1) | (400 MHz; MeOD) δ 8.19 (t, J = 2.5 Hz, 2H), 7.40-7.34 (m, 2H), 7.19- 7.11 (m, 2H), 6.82 (d, J = 5.0 Hz, 1H), 3.91 (t, J = 13.4 Hz, 2H), 3.83 (t, J = 7.2 Hz, 2H), 3.12 (dt, J = 13.9, 7.0 Hz, 1H), 2.41 (tt, J = 13.8, 7.0 Hz, 2H), 1.36 (d, J = 7.0 Hz, 6H). |
| 230 | 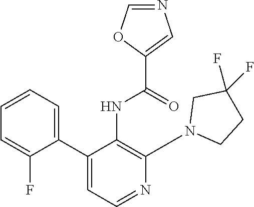 [SMILES: O=C(Nc1c(-c2ccccc2F)ccnc1N1CCC(F)(F)C1)c1cnco1] N-(2-(3,3-difluoropyrrolidin-1- yl)-4-(2-fluorophenyl)pyridin- 3-yl) oxazole-5-carboxamide | 43% | 389.0 (M + 1) | (400 MHz; DMSO-d₆) δ 10.06 (s, 1H), 8.54 (s, 1H), 8.20 (d, J = 5.0 Hz, 1H), 7.77 (s, 1H), 7.39- 7.31 (m, 1H), 7.30-7.21 (m, 1H), 7.17 (td, J = 7.5, 1.0 Hz, 1H), 6.80 (dd, J = 4.9, 0.8 Hz, 1H), 3.92-3.85 (m, 2H), 3.74 (t, J = 7.0 Hz, 2H), 2.48-2.40 (m, 2H). |
| 231 | 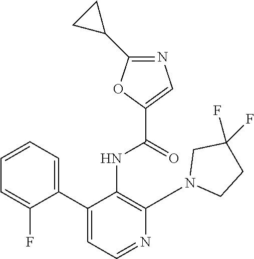 [SMILES: O=C(Nc1c(-c2ccccc2F)ccnc1N1CCC(F)(F)C1)c1cnc(C2CC2)o1] 2-cyclopropyl-N-(2-(3,3- difluoropyrrolidin-1-yl)-4-(2- fluorophenyl)pyridin-3-yl)oxazole-5- carboxamide | 54% | 429.0 (M + 1) | (400 MHz; CDCl₃) δ 8.27 (d, J = 5.0 Hz, 1H), 7.47-7.45 (m, 1H), 7.36 (td, J = 6.7, 1.7 Hz, 2H), 7.22 (td, J = 7.5, 0.8 Hz, 1H), 7.11 (t, J = 9.3 Hz, 1H), 6.79 (d, J = 5.0 Hz, 1H), 3.96-3.92 (m, 2H), 3.90-3.85 (m, 2H), 2.46- 2.36 (m, 2H), 2.14-2.08 (m, 1H), 1.18-1.16 (m, 4H). |
| 232 | 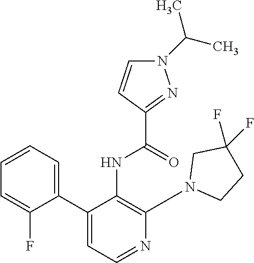 [SMILES: CC(C)n1ccc(C(=O)Nc2c(-c3ccccc3F)ccnc2N2CCC(F)(F)C2)n1] N-(2-(3,3-difluoropyrrolidin-1- yl)-4-(2-fluorophenyl)pyridin- 3-yl)-1-isopropyl-1H-pyrazole-3- carboxamide | 1% | 430.1 (M + 1) | (400 MHz; MeOD) δ 8.13 (d, J = 6.3 Hz, 1H), 7.70 (d, J = 2.4 Hz, 1H), 7.48-7.39 (m, 2H), 7.25- 7.18 (m, 2H), 7.13 (d, J = 6.2 Hz, 1H), 6.63 (d, J = 2.4 Hz, 1H), 4.59 (dt, J = 13.4, 6.7 Hz, 1H), 4.17 (t, J = 12.3 Hz, 2H), 4.01 (t, J = 7.3 Hz, 2H), 2.63-2.52 (m, 2H), 1.51 (d, J = 6.7 Hz, 6H). |
| 233 | 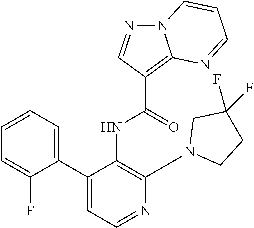 [SMILES: O=C(Nc1c(-c2ccccc2F)ccnc1N1CCC(F)(F)C1)c1cnn2cccnc12] N-(2-(3,3-difluoropyrrolidin-1- yl)-4-(2-fluorophenyl)pyridin- 3-yl)pyrazolo[1,5-a]pyrimidine-3- carboxamide | 17% | 439.0 (M + 1) | (400 MHz; DMSO-d₆) δ 9.35 (s, 1H), 9.30 (dd, J = 7.0, 1.5 Hz, 1H), 8.82 (dd, J = 4.2, 1.5 Hz, 1H), 8.50 (s, 1H), 8.21 (d, J = 5.0 Hz, 1H), 7.38 (td, J = 7.6, 1.5 Hz, 1H), 7.28 (td, J = 6.4, 3.4 Hz, 2H), 7.18-7.11 (m, 2H), 6.82 (d, J = 4.9 Hz, 1H), 3.91 (t, J = 13.5 Hz, 2H), 3.76 (t, J = 7.3 Hz, 2H), 2.44-2.34 (m, 2H). |
| 234 | 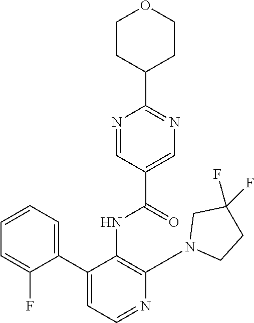 [SMILES: O=C(Nc1c(-c2ccccc2F)ccnc1N1CCC(F)(F)C1)c1cnc(C2CCOCC2)nc1] N-(2-(3,3-difluoropyrrolidin-1- yl)-4-(2-fluorophenyl)pyridin- 3-yl)-2-(tetrahydro-2H-pyran-4- yl)pyrimidine-5-carboxamide | 47% | 484.0 (M + 1) | (400 MHz; CDCl₃) δ 8.88 (s, 2H), 8.29 (d, J = 5.0 Hz, 1H), 7.66-7.63 (m, 1H), 7.36 (dd, J = 6.8, 6.2 Hz, 2H), 7.23 (td, J = 7.5, 1.0 Hz, 1H), 7.14 (t, J = 9.3 Hz, 1H), 6.81 (d, J = 5.0 Hz, 1H), 4.19-4.16 (m, 2H), 3.95- 3.83 (m, 4H), 3.57 (td, J = 11.0, 3.4 Hz, 2H), 3.21-3.14 (m, 1H), 2.40 (tt, J = 13.7, 6.9 Hz, 2H), 2.20-1.95 (m, 4H). |
| 235 | 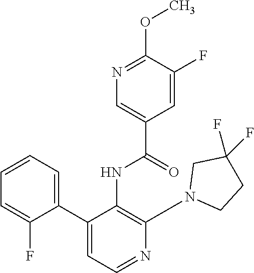 [SMILES: COc1ncc(C(=O)Nc2c(-c3ccccc3F)ccnc2N2CCC(F)(F)C2)cc1F] N-(2-(3,3-difluoropyrrolidin-1- yl)-4-(2-fluorophenyl)pyridin- 3-yl)-5-fluoro-6-methoxynicotinamide | 49% | 447.2 (M + 1) | (400 MHz; DMSO-d₆): δ 9.96 (s, 1H), 8.32 (d, J = 2.0 Hz, 1H), 8.20 (d, J = 5.0 Hz, 1H), 7.85 (dd, J = 11.0, 2.0 Hz, 1H), 7.38-7.33 (m, 1H), 7.31- 7.27 (m, 1H), 7.26-7.21 (m, 1H), 7.17 (td, J = 7.5, 1.0 Hz, 1H), 6.80 (d, J = 5.0 Hz, 1H), 3.99 (s, 3H), 3.92-3.86 (m, 2H), 3.77-3.72 (m, 2H), 2.46-2.40 (m, 2H). |

### Example 236

### Synthesis of N-(2-(3,3-difluoropyrrolidin-1-yl)-4-(1H-pyrazol-5-yl)pyridin-3-yl)-6-(pyrrolidin-1-yl)nicotinamide

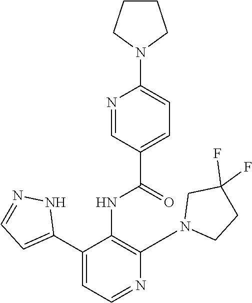 [SMILES: O=C(Nc1c(-c2ccn[nH]2)ccnc1N1CCC(F)(F)C1)c1ccc(N2CCCC2)nc1]

### Step 1. Preparation of N-(2-(3,3-difluoropyrrolidin-1-y1)-4-iodopyridin-3-yl)-6-fluoronicotinamide

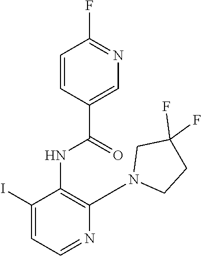 [SMILES: O=C(Nc1c(I)ccnc1N1CCC(F)(F)C1)c1ccc(F)nc1]

A mixture of methyl 6-fluoropyridine-3-carboxylic acid (0.325 g, 2.30 mmol), N-ethyl-N-isopropylpropan-2-amine (0.601 g, 4.65 inmol) and 2-chloro-1-10 mnethylpyridiniumn iodide (0.590 g, 2.31 minol in tetrahydrofuran (10 ml-) was stirred at 25 00 for 30 mr. Then 2-(3,3-difluoropyrrolidin-1-yl)-4-iodo-pyridin-3-amine (0.500 g, 1.54 mmol) was added. The resulting mixture was stirred at 70° C. for 12 h. The reaction mixture was cooled to ambient temperature. Ethyl acetate (30 mL) and water (30 mL) were added and the layers were separated. The aqueous phase was extracted with ethyl acetate (2×20 mL). The combined extracts were washed with brine (20 mL), dried over sodium sulfate and filtered. The filtrate was concentrated under reduced pressure. The residue was purified by flash silica gel chromatography, eluting with a gradient of 55-65% of ethyl acetate in petroleum ether as a colorless solid (0.339 g, 49% yield):¹H NMR (400 MHz, CDCl₃) δ 8.83 (s, 1H), 8.48-8.33 (m, 1H), 7.77 (d, J=5.1 Hz, 1H), 7.57-7.40 (m, 1H), 7.28 (d, J=5.1 Hz, 1H), 7.12 (dd, J=2.6, 8.4 Hz, 1H), 3.97-3.68 (m, 4H), 2.36 (t, J=7.0 Hz, 2H); MS (ES+) m/z 448.9 (M+1).

### Step 2. Preparation of N-(2-(3,3-difluoropyrrolidin-1-yl)-4-iodopyridin-3-yl)-6-(pyrrolidin-1-yl)nicotinamide

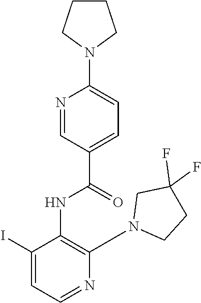 [SMILES: O=C(Nc1c(I)ccnc1N1CCC(F)(F)C1)c1ccc(N2CCCC2)nc1]

To a solution of N-[2-(3,3-difluoropyrrolidin-1-yl)-4-iodo-3-pyridyl]-6-fluoro-pyridine-3-carboxamide (0.0300 g, 0.0669 mmol) and pyrrolidine (0.00800 g, 0.112 mmol) in dimethylsulfoxide (2 mL) was added N-ethyl-N-isopropylpropan-2-amine (0.0260 g, 0.201 mmol). The mixture was stirred at 80° C. for 12 h. The reaction mixture was cooled to ambient temperature. Ethyl acetate (30 mL) and water (30 mL) were added and layers were separated. The aqueous phase was extracted with ethyl acetate (2×20 mL). The combined extracts were washed with brine (20 mL), dried over sodium sulfate and filtered. The filtrate was concentrated under reduced pressure and the residue was purified by reversed phase column chromatography, eluting with a gradient of 60-70% of acetonitrile in water containing 0.1% formic acid, to afford the title compound as a colorless solid (0.0300 g, 90% yield): ¹H NMR (400 MHz, CDCl₃) δ 8.81 (s, 1H), 8.06 (dd, J=1.6, 8.8 Hz, 1H), 7.73 (d, J=5.4 Hz, 1H), 7.47-7.33 (m, 1H), 7.24 (d, J=5.2 Hz, 1H), 6.48 (d, J=8.8 Hz, 1H), 3.96-3.73 (m, 4H), 3.57 (s, 4H), 2.41-2.25 (m, 2H), 2.07 (s, 4H); MS (ES+) m/z 499.9 (M+1).

### Step 3. Preparation of N-(2-(3,3-difluoropyrrolidin-1-yl)-4-(1H-pyrazol-5-yl)pyridin-3-yl)-6-(pyrrolidin-1-yl)nicotinamide

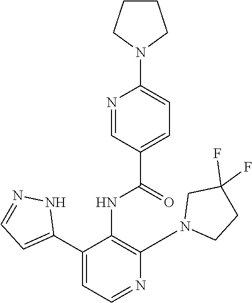 [SMILES: O=C(Nc1c(-c2ccn[nH]2)ccnc1N1CCC(F)(F)C1)c1ccc(N2CCCC2)nc1]

To a mixture of N-[2-(3,3-difluoropyrrolidin-1-yl)-4-iodo-3-pyridyl]-6-pyrrolidin-1-yl-pyridine-3-carboxamide (0.0300 g, 0.0600 mmol), 3-(4,4,5,5-tetramethyl-1,3,2-dioxaborolan-2-yl)-1H-pyrazole (0.0240 g, 0.124 mmol) and potassium carbonate (0.0250 g, 0.181 mmol) in dioxane (2 mL) and water (0.2 mL) was added [1,1′-bis(diphenylphosphino)ferrocene]dichloropalladium(II) (0.00300 g, 0.00410 mmol). The mixture was stirred at 100° C. for 2 h under a nitrogen atmosphere. The reaction mixture was cooled to ambient temperature. Ethyl acetate (20 mL) and water (20 mL) were added and layers were separated. The aqueous phase was extracted with ethyl acetate (20 mL×2). The combined extracts were washed with brine (30 mL), dried over sodium sulfate and filtered. The filtrate was concentrated under reduced pressure and the residue was purified by prep-HPLC, eluting with a gradient of 29-59% of acetonitrile in water containing 10 mM ammonium bicarbonate, to afford the title compound as a grey solid (0.00650 g, 23% yield): ¹H NMR (400 MHz, DMSO-d₆) δ 5 13.26-12.98 (m, 1H), 9.73 (s, 1H), 8.72 (s, 1H), 8.10 (d, J=2.3 Hz, 1H), 8.01 (d, J=8.8 Hz, 1H), 7.85-7.64 (m, 1H), 7.21 (s, 1H), 6.64-6.43 (m, 2H), 3.99-3.64 (m, 4H), 3.45 (s, 4H), 2.46-2.27 (m, 2H), 1.96 (s, 4H); MS (ES+) m/z 440.1 (M+1).

### Example 237-240

In a similar manner as described in Example 236, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 237 | 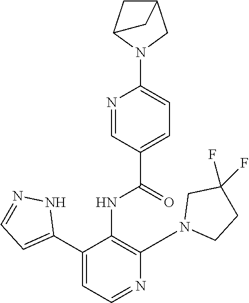 [SMILES: O=C(Nc1c(-c2ccn[nH]2)ccnc1N1CCC(F)(F)C1)c1ccc(N2CC3CC2C3)nc1] 6-(2-azabicyclo[2.1.1] hexan-2-yl)-N-(2-(3,3- difluoropyrrolidin-1- yl)-4-(1H-pyrazol-5- yl)pyridin-3-yl)nicotinamide | 33% | 452.1 (M + 1) | (400 MHz; DMSO-d₆) δ 13.15-13.12 (m, 1H), 9.74 (s, 1H), 8.70 (s, 1H), 8.14-8.10 (m, 1H), 7.99 (dd, J = 8.7, 2.0 Hz, 1H), 7.74-7.73 (m, 1H), 7.22-7.21 (m, 1H), 6.65 (d, J = 8.7 Hz, 1H), 6.56 (s, 1H), 4.87-4.86 (m, 1H), 3.95-3.66 (m, 4H), 3.34 (s, 2H), 3.12- 2.90 (m, 1H), 2.46-2.35 (m, 2H), 2.02-1.98 (m, 2H), 1.37 (dd, J = 4.3, 1.6 Hz, 2H). |
| 238 | 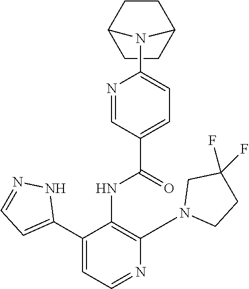 [SMILES: O=C(Nc1c(-c2ccn[nH]2)ccnc1N1CCC(F)(F)C1)c1ccc(N2C3CCC2CC3)nc1] 6-(7-azabicyclo[2.2.1] heptan-7-yl)-N-(2-(3,3- difluoropyrrolidin-1- yl)-4-(1H-pyrazol-5- yl)pyridin-3-yl)nicotinamide | 30% | 466.2 (M + 1) | (400 MHz; DMSO-d₆) δ 13.3-13.1 (broad s, 1H), 8.72 (s, 1H), 8.11 (dd, J = 4.7, 0.5 Hz, 1H), 8.03- 8.01 (m, 1H), 7.70 (s, 1H), 7.18-7.17 (m, 1H), 6.93 (d, J = 8.8 Hz, 1H), 6.56 (s, 1H), 4.61 (s, 2H), 3.94-3.66 (m, 4H), 2.46-2.33 (m, 4H), 1.69- 1.67 (m, 3H), 1.49 (d, J = 7.0 Hz, 3H). |
| 239 | 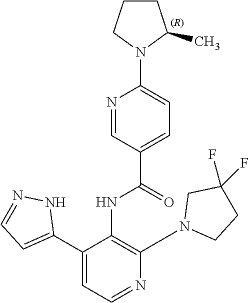 (R)-N-(2-(3,3-difluoropyrrolidin-1- yl)-4-(1H-pyrazol-5-yl)pyridin-3-yl)-6-(2- methylpyrrolidin-1-yl)nicotinamide | 54% | 454.1 (M + 1) | (400 MHz; DMSO-d₆) δ 13.15-13.11 (m, 1H), 9.73 (s, 1H), 8.74-8.71 (m, 1H), 8.12-8.10 (m, 1H), 8.01 (dd, J = 8.8, 2.0 Hz, 1H), 7.74-7.72 (m, 1H), 7.24-7.20 (m, 1H), 6.58-6.52 (m, 2H), 4.24-4.20 (m, 1H), 3.95- 3.66 (m, 5H), 3.57-3.52 (m, 1H), 2.45-2.34 (m, 2H), 2.10-1.94 (m, 3H), 1.73-1.69 (m, 1H), 1.19 (d, J = 6.2 Hz, 3H). |
| 240 | 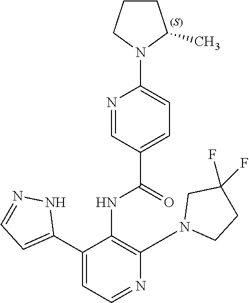 (S)-N-(2-(3,3-difluoropyrrolidin-1- yl)-4-(1H-pyrazol-5-yl)pyridin-3-yl)-6-(2- methylpyrrolidin-1-yl)nicotinamide | 46% | 454.3 (M + 1) | (400 MHz; DMSO-d₆) δ 13.15-13.12 (m, 1H), 9.74 (s, 1H), 8.73 (s, 1H), 8.11 (d, J = 4.5 Hz, 1H), 8.01 (dd, J = 8.8, 1.8 Hz, 1H), 7.74-7.69 (m, 1H), 7.23-7.19 (m, 1H), 6.54 (t, J = 7.6 Hz, 2H), 4.23-4.21 (m, 1H), 3.96-3.66 (m, 5H), 3.57- 3.51 (m, 1H), 2.45-2.35 (m, 2H), 2.10-1.95 (m, 3H), 1.72-1.70 (m, 1H), 1.19 (d, J = 6.2 Hz, 3H). |

### Example 241

### Synthesis of N-(2-(3,3-difluoropyrrolidin-1-yl)-4-(1H-pyrazol-5-yl)pyridin-3-yl)-4-isopropylbenzamide

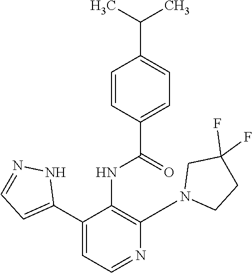 [SMILES: CC(C)c1ccc(C(=O)Nc2c(-c3ccn[nH]3)ccnc2N2CCC(F)(F)C2)cc1]

### Step 1. Preparation of 4-bromo-N-(2-(3,3-difluoropyrrolidin-1-yl)-4-iodopyridin-3-yl)benzamide

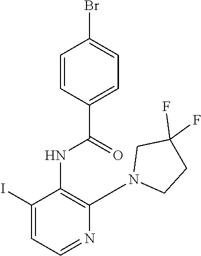 [SMILES: O=C(Nc1c(I)ccnc1N1CCC(F)(F)C1)c1ccc(Br)cc1]

A mixture of 4-bromobenzoic acid (1.86 g, 9.25 mmol), 2-chloro-1-methyl-pyridin-1-ium; iodide (6.29 g, 24.6 mmol) and N-ethyl-N-isopropylpropan-2-amine (3.18 g, 24.6 mmol) in tetrahydrofuran (20 mL) was stirred at 25° C. for 0.5 h. To the mixture was added 2-(3,3-difluoropyrrolidin-1-yl)-4-iodopyridin-3-amine (2.00 g, 6.15 mmol).

The mixture was stirred at 65° C. for 12 h. After cooling to ambient temperature, the mixture was concentrated under reduced pressure. The residue was purified by flash silica gel chromatography, eluting with a gradient of 0-25% of ethyl acetate in petroleum ether to afford the title compound as a colorless solid (0.900 g, 26% yield): ¹H NMR (400 MHz, DMSO-d₆) δ 10.22 (s, 1H), 7.94 (d, J=8.4 Hz, 2H), 7.80 (d, J=8.4 Hz, 2H), 7.75 (d, J=5.2 Hz, 1H), 7.32 (d, J=5.2 Hz, 1H), 4.01-3.85 (m, 1H), 3.83-3.67 (m, 2H), 3.64-3.53 (m, 1H), 2.44-2.29 (m, 2H).

### Step 2. Preparation of 4-bromo-N-(2-(3,3-difluoropyrrolidin-1-yl)-4-(1H-pyrazol-5-yl)pyridin-3-yl)benzamide

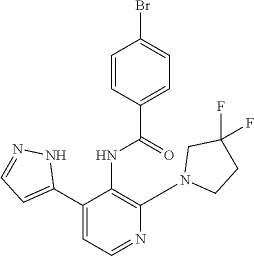 [SMILES: O=C(Nc1c(-c2ccn[nH]2)ccnc1N1CCC(F)(F)C1)c1ccc(Br)cc1]

To a mixture of 4-bromo-N-(2-(3,3-difluoropyrrolidin-1-yl)-4-iodopyridin-3-yl)benzamide (0.800 g, 1.57 mmol), 3-(4,4,5,5-tetramethyl-1,3,2-dioxaborolan-2-yl)-1H-pyrazole (0.307 g, 1.58 mmol), potassium carbonate (0.545 g, 3.94 mmol) and [1,1′-bis(diphenylphosphino)ferrocene]dichloropalladium(II) (0.346 g, 0.473 mmol), under a nitrogen atmosphere, was added dioxane (9 mL) and water (3 mL). The mixture was stirred at 50° C. for 1 h. After cooling to ambient temperature, the mixture was concentrated under reduced pressure. The residue was purified by flash silica gel chromatography twice, eluting with 50-65% of ethyl acetate in petroleum ether, then eluting with 50-60% of ethyl acetate in petroleum ether. The title compound was afforded as a yellow solid (0.116 g, 16% yield): ¹H NMR (400 MHz, DMSO-d₆) δ 13.11 (s, 1H), 10.09 (s, 1H), 8.12 (d, J=5.2 Hz, 1H), 7.92 (d, J=8.2 Hz, 2H), 7.82-7.68 (m, 3H), 7.21 (d, J=5.2 Hz, 1H), 6.57 (s, 1H), 3.97-3.60 (m, 4H), 2.40 (td, J=6.8, 13.2 Hz, 2H).

### Step 3. Preparation of N-(2-(3,3-difluoropyrrolidin-1-yl)-4-(1H-pyrazol-5-yl)pyridin-3-yl)-4-(prop-1-en-2-yl)benzamide

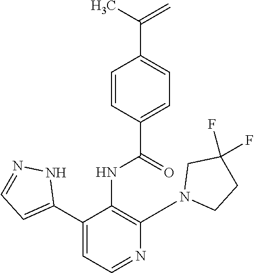 [SMILES: C=C(C)c1ccc(C(=O)Nc2c(-c3ccn[nH]3)ccnc2N2CCC(F)(F)C2)cc1]

To a solution of 4-bromo-N-(2-(3,3-difluoropyrrolidin-1-yl)-4-(1H-pyrazol-5-yl)pyridin-3-yl)benzamide (0.116 g, 0.259 mmol), 4,4,5,5-tetramethyl-2-(prop-1-en-2-yl)-1,3,2-dioxaborolane (0.132 g, 0.786 umol) and cesium carbonate (0.254 g, 0.780 mmol) in dioxane (2 mL) and water (0.4 mL) was added [1,1′-bis(diphenylphosphino)ferrocene]dichloropalladium(II) (0.038 g, 0.0519 mmol) at 25° C. under an atmosphere of nitrogen. Then the mixture was stirred at 90° C. for 12 h. After cooling to ambient temperature, the mixture was concentrated under reduced pressure. The residue was purified by flash silica gel chromatography, eluting with a gradient of 50-64% of ethyl acetate in petroleum ether, to afford the title compound as a yellow solid (0.0400 g, 32% yield): ¹H NMR (400 MHz, DMSO-d₆) δ 13.11 (s, 1H), 10.03 (s, 1H), 8.12 (d, J=5.2 Hz, 1H), 7.97 (d, J=8.4 Hz, 2H), 7.73 (s, 1H), 7.66 (d, J=8.4 Hz, 2H), 7.22 (d, J=5.2 Hz, 1H), 6.58 (s, 1H), 5.57 (s, 1H), 5.23 (s, 1H), 3.98-3.63 (m, 4H), 2.46-2.29 (m, 2H), 2.16 (s, 3H).

### Step 4. Preparation of N-(2-(3,3-difluoropyrrolidin-1-yl)-4-(1H-pyrazol-5-yl)pyridin-3-yl)-4-isopropylbenzamide

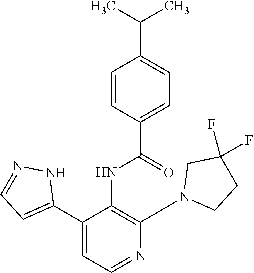 [SMILES: CC(C)c1ccc(C(=O)Nc2c(-c3ccn[nH]3)ccnc2N2CCC(F)(F)C2)cc1]

To a solution of N-(2-(3,3-difluoropyrrolidin-1-yl)-4-(1H-pyrazol-5-yl)pyridin-3-yl)-4-(prop-1-en-2-yl)benzamide (0.0400 g, 0.0980 mmol) in methanol (2 mL) was added palladium on carbon (10 mg, 10% purity) under a nitrogen atmosphere. The suspension was degassed and purged with hydrogen 3 times. The mixture was stirred under hydrogen (15 Psi) at 25° C. for 1 h. The mixture was filtered and the filtrate was concentrated under reduced pressure. The residue was purified by prep-HPLC, eluting with a gradient of 28-58% of acetonitrile in water containing 0.2% formic acid to afford the title compound as a colorless solid (0.0161 g, 39% yield): ¹H NMR (400 MHz, DMSO-d₆) δ 13.14 (s, 1H), 9.98 (s, 1H), 8.12 (d, J=5.0 Hz, 1H), 7.91 (d, J=8.0 Hz, 2H), 7.70 (d, J=1.0 Hz, 1H), 7.40 (d, J=8.0 Hz, 2H), 7.19 (s, 1H), 6.56 (s, 1H), 3.95-3.63 (m, 4H), 2.97 (td, J=6.8, 13.8 Hz, 1H), 2.39 (dt, J=6.8, 13.8 Hz, 2H), 1.24 (d, J=6.8 Hz, 6H); MS (ES+) m/z 412.0 (M+1).

### Example 242

### Synthesis of N-(2-(3,3-difluoropyrrolidin-1-yl)-4-(1H-pyrazol-5-yl)pyridin-3-yl)-4-methoxybenzamide

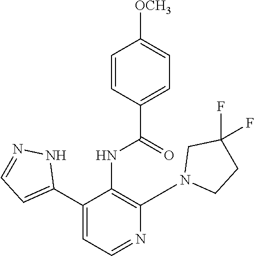 [SMILES: COc1ccc(C(=O)Nc2c(-c3ccn[nH]3)ccnc2N2CCC(F)(F)C2)cc1]

### Step 1. Preparation of 4-bromo-N-(2-(3,3-difluoropyrrolidin-1-yl)-4-(1-(tetrahydro-2H-pyran-2-yl)-1H-pyrazol-3-yl)pyridin-3-yl)benzamide

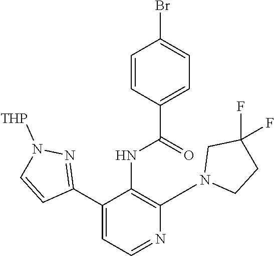

To a mixture of 4-bromo-N-(2-(3,3-difluoropyrrolidin-1-yl)-4-iodopyridin-3-yl)benzamide (0.920 g, 1.81 mmol), 1-(tetrahydro-2H-pyran-2-yl)-3-(4,4,5,5-tetramethyl-1,3,2-dioxaborolan-2-yl)-1H-pyrazole (0.504 g, 1.81 mmol), potassium carbonate (0.626 g, 4.53 mmol) and [1,1′-bis(diphenylphosphino)ferrocene]dichloropalladium(II) (0.398 g, 0.544 mmol) was added dioxane (9 mL) and water (3 mL). The mixture was stirred at 50° C. for 12 h. After cooling to ambient temperature, the mixture was concentrated under reduced pressure. The residue was purified by flash silica gel chromatography, eluting with a gradient of 0-39% of ethyl acetate in petroleum ether, to afford the title compound as a colorless oil (0.100 g, 10% yield): ¹H NMR (400 MHz, DMSO-d₆) δ 9.88 (s, 1H), 8.23 (d, J=5.0 Hz, 1H), 7.77-7.67 (m, 2H), 7.67-7.57 (m, 2H), 7.44 (d, J=1.6 Hz, 1H), 6.81 (d, J=5.0 Hz, 1H), 6.24 (s, 1H), 4.99 (dd, J=2.0, 10.0 Hz, 1H), 3.93 (d, J=10.4 Hz, 2H), 3.81 (dd, J=1.6, 4.2 Hz, 2H), 3.70 (s, 1H), 3.49-3.38 (m, 1H), 2.44 (td, J=7.0, 14.2 Hz, 2H), 2.24 (d, J=10.8 Hz, 1H), 1.91 (s, 1H), 1.66 (d, J=11.6 Hz, 1H), 1.59-1.41 (m, 3H).

### Step 2. Preparation of N-(2-(3,3-difluoropyrrolidin-1-yl)-4-(1-(tetrahydro-2H-pyran-2-yl)-1H-pyrazol-3-yl)pyridin-3-yl)-4-(4,4,5,5-tetramethyl-1,3,2-dioxaborolan-2-yl)benzamide

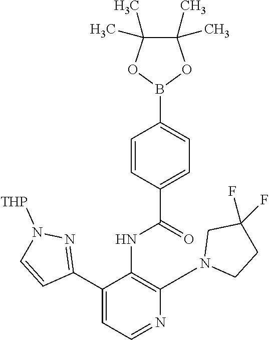

To a solution of 4-bromo-N-(2-(3,3-difluoropyrrolidin-1-yl)-4-(1-(tetrahydro-2H-pyran-2-yl)-1H-pyrazol-3-yl)pyridin-3-yl)benzamide (0.0900 g, 0.170 mmol), 4,4,4′,4′,5,5,5′,5′-octamethyl-2,2′-bi(1,3,2-dioxaborolane) (0.0560 g, 0.221 mmol) and potassium acetate (0.0340 g, 0.346 mmol) in dioxane (2 mL) was added [1,1′-bis(diphenylphosphino)ferrocene]dichloropalladium(II) (0.013 g, 0.018 mmol) at 25° C. Then the mixture was stirred at 80° C. for 12 h. After cooling to ambient temperature, the mixture was poured into water (20 mL). The mixture was extracted with ethyl acetate (3×20 mL). The combined organic layer was washed with brine (20 mL), dried over sodium sulfate, filtered and the filtrate was evaporated under reduced pressure to give to afford the title compound as a brown oil (0.0900 g, crude): ¹H NMR (400 MHz, DMSO-d₆) δ 9.86 (s, 1H), 8.23 (d, J=5.0 Hz, 1H), 7.93 (s, 1H), 7.83-7.69 (m, 3H), 7.45-7.41 (m, 1H), 6.81 (d, J=4.8 Hz, 1H), 6.25 (s, 1H), 5.01 (dd, J=2.0, 9.8 Hz, 1H), 3.98-3.89 (m, 2H), 3.88-3.75 (m, 2H), 3.74-3.65 (m, 1H), 3.48-3.39 (m, 1H), 2.47-2.35 (m, 2H), 2.23 (d, J=9.8 Hz, 1H), 1.95-1.85 (m, 1H), 1.66 (d, J=12.2 Hz, 1H), 1.57-1.43 (m, 3H), 1.30 (s, 1OH), 1.07 (s, 2H).

### Step 3. Preparation of N-(2-(3,3-difluoropyrrolidin-1-yl)-4-(1-(tetrahydro-2H-pyran-2-yl)-1H-pyrazol-3-yl)pyridin-3-yl)-4-hydroxybenzamide

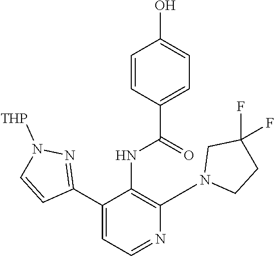

To a solution of N-(2-(3,3-difluoropyrrolidin-1-yl)-4-(1-(tetrahydro-2H-pyran-2-yl)-1H-pyrazol-3-yl)pyridin-3-yl)-4-(4,4,5,5-tetramethyl-1,3,2-dioxaborolan-2-yl)benzamide (0.0900 g, 0.155 mmol) in dichloromethane (4 mL) was added hydrogen peroxide (1.20 mL, 30% purity) at 0 0C. The mixture was stirred at 25° C. for 12 h. The reaction mixture was quenched by saturated aqueous sodium sulfite (20 mL) at 0° C., and then extracted with dichloromethane (3×20 mL). The combined organic layers were washed with brine (20 mL), dried over sodium sulfate, filtered and concentrated under reduced pressure. The residue was purified by flash silica gel chromatography, eluting with a gradient of 30-66% of ethyl acetate in petroleum ether to afford the title compound as a colorless solid (0.0450 g, 57% yield): ¹H NMR (400 MHz, DMSO-d₆) c 10.08 (s, 1H), 9.49 (s, 1H), 8.21 (d, J=4.8 Hz, 1H), 7.59 (d, J=8.4 Hz, 2H), 7.43 (s, 1H), 6.83-6.75 (m, 3H), 6.24 (s, 1H), 5.01 (d, J=8.8 Hz, 1H), 3.93 (d, J=11.0 Hz, 2H), 3.79 (s, 2H), 3.71 (d, J=6.4 Hz, 1H), 3.50-3.39 (m, 1H), 2.42 (td, J=6.8, 14.2 Hz, 2H), 2.23 (d, J=10.4 Hz, 1H), 1.90 (s, 1H), 1.71-1.62 (m, 1H), 1.57-1.41 (m, 3H).

### Step 4. Preparation of N-(2-(3,3-difluoropyrrolidin-1-yl)-4-(1-(tetrahydro-2H-pyran-2-yl)-1H-pyrazol-3-yl)pyridin-3-yl)-4-methoxybenzamide

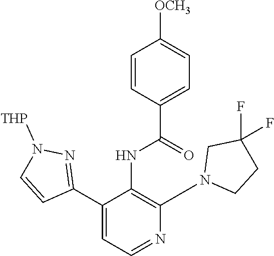

To a solution of N-(2-(3,3-difluoropyrrolidin-1-yl)-4-(1-(tetrahydro-2H-pyran-2-yl)-1H-pyrazol-3-yl)pyridin-3-yl)-4-hydroxybenzamide (0.0200 g, 0.0426 mmol) and potassium carbonate (0.0120 g, 0.0868 mmol) in acetone (1 mL) was added methyl iodide (0.00600 g, 0.0423 mmol) at 0° C. Then the mixture was stirred at 25° C. for 12 h. The mixture was concentrated under reduced pressure. The residue was purified by prep-HPLC, eluting with a gradient of 38-68% of acetonitrile in water containing 0.225% formic acid, to afford the title compound as a colorless solid (0.0100 g, 38% yield): ¹H NMR (400 MHz, CDCl₃) δ 8.24 (d, J=4.8 Hz, 1H), 7.61-7.49 (m, 3H), 6.88 (d, J=8.8 Hz, 2H), 6.73 (d, J=4.4 Hz, 1H), 6.25 (s, 1H), 5.19-5.02 (m, 1H), 4.15 (d, J=10.8 Hz, 1H), 3.89-3.81 (m, 7H), 3.61 (t, J=11.2 Hz, 1H), 2.38 (td, J=6.2, 12.8 Hz, 3H), 2.07 (s, 3H), 1.91 (d, J=13.4 Hz, 2H).

### Step 5. Preparation of N-(2-(3,3-difluoropyrrolidin-1-yl)-4-(1H-pyrazol-5-yl)pyridin-3-yl)-4-methoxybenzamide

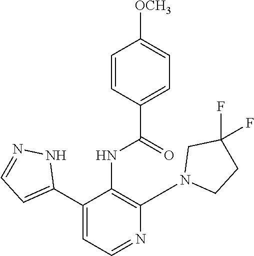 [SMILES: COc1ccc(C(=O)Nc2c(-c3ccn[nH]3)ccnc2N2CCC(F)(F)C2)cc1]

To a solution of N-(2-(3,3-difluoropyrrolidin-1-yl)-4-(1-(tetrahydro-2H-pyran-2-yl)-1H-pyrazol-3-yl)pyridin-3-yl)-4-methoxybenzamide (0.00800 g, 0.0166 mmol) in dichloromethane (1 mL) was added hydrochloric acid/dioxane (4 M, 1 mL) at 25° C. Then the mixture was stirred at 25° C. for 1 h. The mixture was concentrated under 20 reduced pressure. The residue was purified by prep-HPLC, eluting with a gradient of 22-42% of acetonitrile in water containing 0.225% formic acid, to afford the title compound as a gray gum (0.00630 g, 94% yield): ¹H NMR (400 MHz, DMSO-d₆) δ 10.02 (s, 1H), 8.11 (d, J=5.4 Hz, 1H), 7.98 (d, J=8.8 Hz, 2H), 7.70 (d, J=2.0 Hz, 1H), 7.26 (d, J=5.2 Hz, 1H), 7.07 (d, J=8.8 Hz, 2H), 6.60 (d, J=2.2 Hz, 1H), 4.11-3.92 (m, 2H), 3.84 (s, 5H), 2.41 (dd, J=6.6, 13.6 Hz, 2H); MS (ES+) m/z 400.2 (M+1).

### Example 243

### Synthesis of N-(2-(3,3-difluoropyrrolidin-1-yl)-4-(1H-pyrazol-5-yl)pyridin-3-yl)-4-(oxetan-2-yl)benzamide

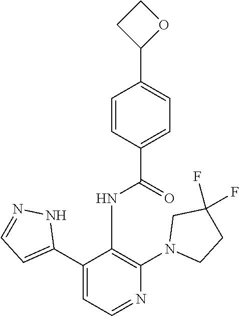 [SMILES: O=C(Nc1c(-c2ccn[nH]2)ccnc1N1CCC(F)(F)C1)c1ccc(C2CCO2)cc1]

### Step 1. Preparation of 4-(oxetan-2-yl)benzoic acid

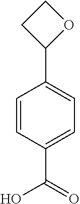 [SMILES: O=C(O)c1ccc(C2CCO2)cc1]

A mixture of methyl 4-(oxetan-2-yl)benzoate (1.20 g, 6.24 mmol) and lithium hydroxide monohydrate (1.31 g, 31.2 mmol) in methanol (20 mL)/water (20 mL) was stirred at 25° C. for 12 h. The reaction mixture was concentrated under reduced pressure. The mixture was adjusted to pH=5 with 1 M hydrochloric acid. The mixture was extracted with dichloromethane (3×200 mL). The combined organic layers were washed with brine (200 mL), dried over sodium sulfate, filtered and concentrated under reduced pressure to afford the title compound as a yellow solid (0.900 g, crude): ¹H NMR (400 MHz, DMSO-d₆) δ 13.63-12.21 (m, 1H), 7.96 (d, J=8.2 Hz, 2H), 7.54 (d, J=8.2 Hz, 2H), 5.80 (t, J=7.2 Hz, 1H), 4.70 (dt, J=5.8, 7.8 Hz, 1H), 4.56 (td, J=5.6, 9.2 Hz, 1H), 3.02 (dtd, J=5.8, 8.2, 10.8 Hz, 1H), 2.55-2.52 (m, 1H).

### Step 2. Preparation of N-(2-(3,3-difluoropyrrolidin-1-yl)-4-iodopyridin-3-yl)-4-(oxetan-2-yl)benzamide

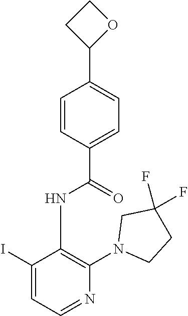 [SMILES: O=C(Nc1c(I)ccnc1N1CCC(F)(F)C1)c1ccc(C2CCO2)cc1]

To a mixture of 4-(oxetan-2-yl)benzoic acid (0.100 g, 0.561 mmol), 2-(3,3-difluoropyrrolidin-1-yl)-4-iodo-pyridin-3-amine (0.182 g, 0.561 mmol), 2-chloro-1-methyl-pyridin-1-ium;iodide (0.172 g, 0.673 mmol) and N,N-diisopropylethylamine (0.217 g, 1.68 mmol) was added tetrahydrofuran (2 mL). The mixture was stirred at 65° C. for 12 h. The reaction mixture was cooled to ambient temperature and concentrated under reduced pressure. The residue was purified by flash silica gel chromatography, eluting with a gradient of 0-50% of ethyl acetate in petroleum ether, then prep-HPLC, eluting with a gradient of 35-65% of acetonitrile in water containing ammonium bicarbonate to afford the title compound as a white solid (0.250 g, 10% yield): ¹H NMR (400 MHz, CDCl₃) δ 8.00 (d, J=8.2 Hz, 2H), 7.78 (d, J=5.2 Hz, 1H), 7.61 (d, J=8.0 Hz, 2H), 7.42 (s, 1H), 7.30 (s, 1H), 5.92 (t, J=7.6 Hz, 1H), 4.90 (dt, J=6.0, 7.8 Hz, 1H), 4.73 (td, J=5.8, 9.2 Hz, 1H), 3.88 (t, J=13.2 Hz, 2H), 3.82 (t, J=7.4 Hz, 2H), 3.14 (dtd, J=5.8, 8.2, 11.0 Hz, 1H), 2.76-2.61 (m, 1H), 2.44-2.29 (m, 2H).

### Step 3. Preparation of N-(2-(3,3-difluoropyrrolidin-1-yl)-4-(1H-pyrazol-5-yl)pyridin-3-yl)-4-(oxetan-2-yl)benzamide

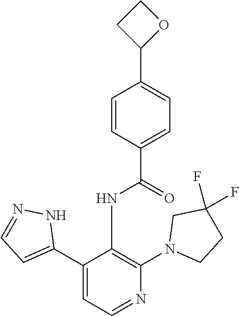 [SMILES: O=C(Nc1c(-c2ccn[nH]2)ccnc1N1CCC(F)(F)C1)c1ccc(C2CCO2)cc1]

To a mixture of N-(2-(3,3-difluoropyrrolidin-1-yl)-4-iodopyridin-3-yl)-4-(oxetan-2-yl)benzamide (0.100 g, 0.206 mmol), 5-(4,4,5,5-tetramethyl-1,3,2-dioxaborolan-2-yl)-1H-pyrazole (0.0599 g, 0.309 mmol), [1,1′-bis(diphenylphosphino)ferrocene]dichloropalladium(II)-dichloromethane (0.0168 g, 0.0206 mmol) and potassium carbonate (0.0569 g, 0.412 mmol) was added dioxane (2 mL) and water (0.4 mL). The mixture was stirred at 100° C. for 12 h under nitrogen atmosphere. The reaction mixture was cooled to ambient temperature and concentrated under reduced pressure. The residue was purified by prep-HPLC, eluting with a gradient of 28-58% of acetonitrile in water containing ammonium bicarbonate to afford the title compound as a colorless solid (0.0585 g, 66% yield): ¹H NMR (400 MHz, DMSO-d₆) δ 13.12 (s, 1H), 10.05 (s, 1H), 8.12 (d, J=4.4 Hz, 1H), 8.01 (d, J=7.8 Hz, 2H), 7.74 (s, 1H), 7.58 (d, J=8.0 Hz, 2H), 7.22 (d, J=4.4 Hz, 1H), 6.58 (s, 1H), 5.81 (t, J=7.4 Hz, 1H), 4.71 (dt, J=5.8, 7.8 Hz, 1H), 4.59 (td, J=5.8, 9.2 Hz, 1H), 4.03-3.61 (m, 4H), 3.10-2.96 (m, 1H), 2.63-2.54 (m, 1H), 2.45-2.34 (m, 2H); MS (ES+) m/z 426.1 (M+1).

### Example 244

### Synthesis of 2-(3-acrylamidoazetidin-1-yl)-N-(2-(3,3-difluoropyrrolidin-1-yl)-4-(2-fluorophenyl)pyridin-3-yl)pyrimidine-5-carboxamide

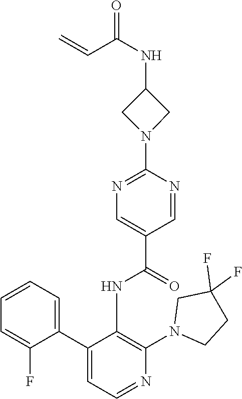 [SMILES: C=CC(=O)NC1CN(c2ncc(C(=O)Nc3c(-c4ccccc4F)ccnc3N3CCC(F)(F)C3)cn2)C1]

Step 1. Preparation of tert-butyl (1-(5-((2-(3,3-difluoropyrrolidin-1-yl)-4-(2-fluorophenyl)pyridin-3-yl)carbamoyl)pyrimidin-2-yl)azetidin-3-yl)carbamate

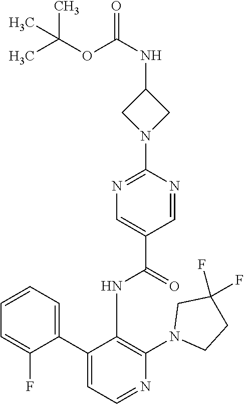 [SMILES: CC(C)(C)OC(=O)NC1CN(c2ncc(C(=O)Nc3c(-c4ccccc4F)ccnc3N3CCC(F)(F)C3)cn2)C1]

To a solution of 2-chloro-N-(2-(3,3-difluoropyrrolidin-1-yl)-4-(2-fluorophenyl)pyridin-3-yl)pyrimidine-5-carboxamide (0.500 g, 1.15 mmol), tert-butyl azetidin-3-ylcarbamate (0.300 g, 1.73 mmol) and N-ethyl-N-isopropylpropan-2-amine (0.447 g, 3.46 mmol) was added dimethylsulfoxide (5 mL). The mixture was stirred at 25° C. for 12 h. The reaction mixture was diluted with water (20 mL) and extracted with ethyl acetate (2×50 mL). The combined organic layers were washed with brine (2×10 mL), dried over sodium sulfate, filtered and concentrated under reduced pressure. The residue was purified by flash silica gel chromatography, eluting with a gradient of 80-90% of ethyl acetate in petroleum ether, to afford the title compound as a yellow solid (0.320 g, 46% yield): ¹H NMR (400 MHz, DMSO-d₆) δ 9.72 (s, 1H), 8.56 (s, 2H), 8.18 (d, J=4.8 Hz, 1H), 7.60 (d, J=7.4 Hz, 1H), 7.38-7.31 (m, 1H), 7.30-7.19 (m, 2H), 7.18-7.13 (m, 1H), 6.78 (d, J=4.8 Hz, 1H), 4.42 (d, J=6.4 Hz, 1H), 4.29 (t, J=8.6 Hz, 2H), 3.90 (dd, J=5.2, 9.4 Hz, 3H), 3.73 (s, 2H), 3.31 (s, 1H), 2.38 (d, J=7.4 Hz, 2H), 1.39 (s, 9H); MS (ES+) m/z 570.4 (M+1).

### Step 2. Preparation of 2-(3-aminoazetidin-1-yl)-N-(2-(3,3-difluoropyrrolidin-1-yl)-4-(2-fluorophenyl)pyridin-3-yl)pyrimidine-5-carboxamide hydrochloride salt

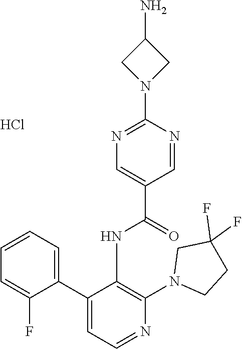 [SMILES: NC1CN(c2ncc(C(=O)Nc3c(-c4ccccc4F)ccnc3N3CCC(F)(F)C3)cn2)C1 ; Cl]

To a solution of tert-butyl (1-(5-((2-(3,3-difluoropyrrolidin-1-yl)-4-(2-fluorophenyl)pyridin-3-yl)carbamoyl)pyrimidin-2-yl)azetidin-3-yl)carbamate (0.300 g, 0.526 mmol) in dichloromethane (5 mL) was added hydrogen chloride in dioxane (4 M, 5 mL). The mixture was stirred at 25° C. for 2 h. The mixture was concentrated under reduced pressure to afford the title compound as a colorless solid (0.260 g, crude): ¹H NMR (400 MHz, DMSO-d₆) δ 510.13-9.75 (m, 1H), 8.63 (s, 2H), 8.50-8.39 (m, 2H), 8.18 (d, J=4.8 Hz, 1H), 7.40-7.28 (m, 2H), 7.26-7.19 (m, 1H), 7.19-7.13 (m, 1H), 6.81 (s, 1H), 4.34 (dd, J=7.0, 9.6 Hz, 2H), 4.13-4.06 (m, 4H), 3.94-3.89 (m, 2H), 3.45-3.42 (m, 1H), 2.43-2.37 (m, 2H); MS (ES+) m/z 470.3 (M+1).

### Step 3. Preparation of 2-(3-acrylamidoazetidin-1-yl)-N-(2-(3,3-difluoropyrrolidin-1-yl)-4-(2-fluorophenyl)pyridin-3-yl)pyrimidine-5-carboxamide

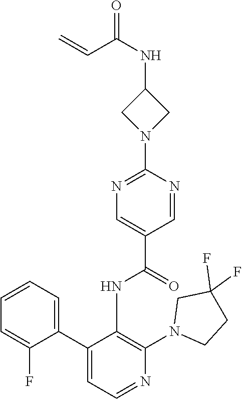 [SMILES: C=CC(=O)NC1CN(c2ncc(C(=O)Nc3c(-c4ccccc4F)ccnc3N3CCC(F)(F)C3)cn2)C1]

To a solution of 2-(3-aminoazetidin-1-yl)-N-(2-(3,3-difluoropyrrolidin-1-yl)-4-(2-fluorophenyl)pyridin-3-yl)pyrimidine-5-carboxamide (0.100 g, 0.197 mmol, hydrochloride) and trimethylamine (0.100 g, 0.988 mmol) in dichloromethane (3 mL) was added 3-chloropropanoyl chloride (0.0130 g, 0.102 mmol) at 0° C. The mixture was stirred at 25° C. for 2 h. The mixture was concentrated under reduced pressure.

The residue was purified by prep-HPLC, eluting with a gradient of 29-59% of acetonitrile in water containing ammonium bicarbonate to afford the title compound as a colorless solid (0.0218 g, 20% yield): ¹H NMR (400 MHz, DMSO-d₆) δ 9.74 (s, 1H), 8.80 (d, J=7.2 Hz, 1H), 8.58 (s, 2H), 8.18 (d, J=4.8 Hz, 1H), 7.38-7.32 (m, 1H), 7.30-7.20 (m, 2H), 7.19-7.13 (m, 1H), 6.78 (d, J=4.8 Hz, 1H), 6.25-6.08 (m, 2H), 5.69-5.62 (m, 1H), 4.75-4.61 (m, 1H), 4.37 (t, J=8.6 Hz, 2H), 3.94 (dd, J=5.2, 9.8 Hz, 2H), 3.91-3.80 (m, 2H), 3.74 (s, 2H), 2.44-2.37 (m, 2H); MS (ES+) m/z 524.3 (M+1); MS (ES+) m/z 524.3 (M+1).

### Example 245

### Synthesis of 6-acryloyl-N-(2-(3,3-difluoropyrrolidin-1-yl)-4-(2-fluorophenyl)pyridin-3-yl)-2,6-diazaspiro[3.3]heptane-2-carboxamide

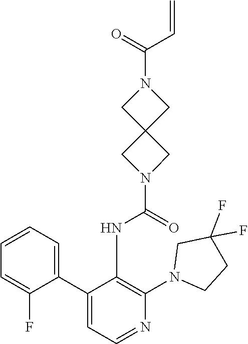 [SMILES: C=CC(=O)N1CC2(C1)CN(C(=O)Nc1c(-c3ccccc3F)ccnc1N1CCC(F)(F)C1)C2]

To a solution of N-(2-(3,3-difluoropyrrolidin-1-yl)-4-(2-fluorophenyl)pyridin-3-yl)-2,6-diazaspiro[3.3]heptane-2-carboxamide (0.0700 g, 0.131 mmol, trifluoroacetate) and trimethylamine (0.0670 g, 0.662 mmol) in dichloromethane (5 mL) at 0° C. was added acryloyl chloride (0.00600 g, 0.0660 mmol). The mixture was stirred at 25° C. for 1 h. The mixture was concentrated under reduced pressure. The residue was purified by prep-HPLC, eluting with a gradient of 21-54% of acetonitrile in water containing ammonium bicarbonate, to afford the title compound as a colorless solid (0.0159 g, 24% yield): ¹H NMR (400 MHz, DMSO-d₆) δ 8.09 (d, J=4.8 Hz, 1H), 7.93 (s, 1H), 7.51-7.43 (m, 1H), 7.32 (d, J=9.2 Hz, 1H), 7.29-7.23 (m, 2H), 6.71 (d, J=5.2 Hz, 1H), 6.32-6.19 (m, 1H), 6.11-5.99 (m, 1H), 5.65 (dd, J=2.4, 10.2 Hz, 1H), 4.21 (s, 2H), 3.91 (s, 4H), 3.74 (t, J=6.8 Hz, 2H), 3.67 (s, 4H), 2.46-2.39 (m, 2H); MS (ES+) m/z 472.3 (M+1).

### Example 246

### Synthesis of N-(4-(2,5-difluorophenyl)-6-(3,3-difluoropyrrolidin-1-yl)pyrimidin-5-yl)-5-fluoro-6-methoxynicotinamide

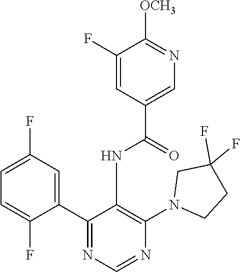 [SMILES: COc1ncc(C(=O)Nc2c(-c3cc(F)ccc3F)ncnc2N2CCC(F)(F)C2)cc1F]

This compound was prepared in a similar manner as described in Example 217, utilizing the appropriately substituted starting materials and intermediates, to afford the title compound as a colorless solid: ¹H NMR (400 MHz, DMSO-d₆) δ 3 10.10 (s, 1H), 8.61 (s, 1H), 8.35 (d, J=2.0 Hz, 1H), 7.89 (dd, J=2.0, 11.2 Hz, 1H), 7.36-7.22 (m, 2H), 7.18 (dt, J=2.8, 5.6 Hz, 1H), 4.28-4.07 (m, 1H), 4.04-3.82 (m, 5H), 3.79-3.64 (m, 1H), 2.46 (d, J=7.2 Hz, 2H); MS (ES+) m/z 466.1 (M+1).

### Example 247

### Synthesis of 2-(cyclopropylmethoxy)-N-(2′-(3,3-difluoropyrrolidin-1-yl)-[2,4′-bipyridin]-3′-yl)pyrimidine-5-carboxamide

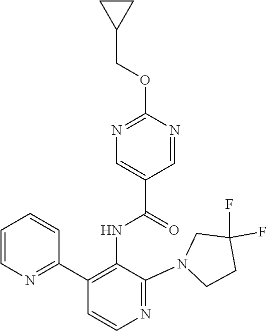 [SMILES: O=C(Nc1c(-c2ccccn2)ccnc1N1CCC(F)(F)C1)c1cnc(OCC2CC2)nc1]

### Step 1. Preparation of tert-butyl (2′-chloro-[2,4′-bipyridin]-3′-yl)-carbamate

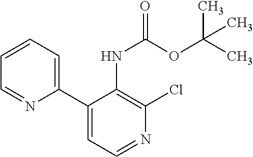 [SMILES: CC(C)(C)OC(=O)Nc1c(-c2ccccn2)ccnc1Cl]

To a mixture of tert-butyl (2-chloro-4-iodopyridin-3-yl)carbamate (0.400 g, 1.13 mmol), pyridin-2-ylzinc(II) bromide (0.5 M, 5.64 mL) and tetrakis[triphenylphosphine]palladium(0) (0.130 g, 0.113 mmol) was added tetrahydrofuran (5 mL). The mixture was stirred at 90° C. for 4 h under an atmosphere of argon under microwave irradiation. After cooling to ambient temperature, the combined mixture (four batches) was added to saturated ammonium chloride (80 mL), extracted with ethyl acetate (3×50 mL), dried and filtered. The filtrate was concentrated in vacuo. The residue was purified by column chromatography on silica gel, eluting with 33% of ethyl acetate in petroleum ether, followed by column chromatography on silica gel, eluting with dichloromethane. The residue was dissolved in dichloromethane (10 mL) at 25° C. Hexane (20 mL) was added and the mixture was stirred for 1 h. The precipitate was filtered. The filter cake was collected and dried in vacuo to afford the title compound as a light-yellow solid (0.600 g, 1.88 mmol, 48% yield): ¹H NMR (400 MHz, CDCl₃) δ 88.73 (d, J=4.8 Hz, 1H), 8.32 (d, J=5.2 Hz, 1H), 7.85 (dt, J=7.6, 1.6 Hz, 1H), 7.78 (br s, 1H), 7.68 (d, J=8.0 Hz, 1H), 7.45 (d, J=5.2 Hz, 1H), 7.36 (ddd, J=7.6, 4.8, 0.8 Hz, 1H), 1.34 (s, 9H).

### Step 2. Preparation of 2′-(3,3-difluoropyrrolidin-1-yl)-[2,4′-bipyridin]-3′-amine

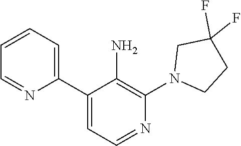 [SMILES: Nc1c(-c2ccccn2)ccnc1N1CCC(F)(F)C1]

A mixture of tert-butyl (2′-chloro-[2,4′-bipyridin]-3′-yl)carbamate (0.200 g, 0.654 mmol), 3,3-difluoropyrro-lidine hydrochloride (0.600 g, 4.18 mmol) and N,N-diisopropylethylamine (0.803 g, 6.21 mmol) in 1-methyl-2-pyrrolidinone (8 mL) in a sealed microwave tube (20 mL) was stirred at 230° C. in a microwave reactor for 4 h. After cooling to ambient temperature, the mixture was poured into water (40 mL). The mixture was extracted with ethyl acetate/petroleum ether (3×200 mL, 9/1). The organic phase was dried over sodium sulfate, filtered and concentrated in vacuo. The residue was purified by reverse phase HPLC, eluting with 0.1% ammonium hydroxide, to afford the title compound as a yellow solid (0.450 g, 50% yield): ¹H NMR (400 MHz, CDCl₃) δ 8.66 (d, J=4.8 Hz, 1H), 7.88-7.70 (m, 3H), 7.25 (s, 1H), 7.19 (d, J=5.2 Hz, 1H), 5.99 (s, 2H), 3.67 (t, J=13.2 Hz, 2H), 3.51 (t, J=7.2 Hz, 2H), 2.44 (tt, J=7.2, 14.4 Hz, 2H).

### Step 3. Preparation of 2-chloro-N-(2′-(3,3-difluoropyrrolidin-1-yl)-[2,4′-bipyridin]-3′-yl)pyrimidine-5-carboxamide

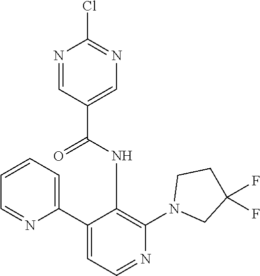 [SMILES: O=C(Nc1c(-c2ccccn2)ccnc1N1CCC(F)(F)C1)c1cnc(Cl)nc1]

To a mixture of 2-chloropyrimidine-5-carboxylic acid (0.100 g, 0.631 mmol) in tetrahydrofuran (4 mL) was added N,N-diisopropylethylamine (0.408 g, 3.15 mmol), 2-chloro-1-methyl-pyridin-1-ium iodide (0.322 g, 1.26 mmol) and 2-(3,3-difluoropyrrolidin-1-yl)-4-(2-pyridyl)pyridin-3-amine (0.174 g, 0.631 mmol). The mixture was stirred 70° C. for 12 h. Two batches were combined and added to ice-water (5 mL). The mixture was extracted with ethyl acetate (4×10 mL), dried over sodium sulfate, filtered and concentrated in vacuo. The residue was purified by column chromatograph on silica gel, eluting with a 5:1 mixture of dichloromethane and ethyl acetate containing 0.6% of triethylamine, to afford the title compound as a yellow solid (0.100 g, 34% yield): ¹H NMR (400 MHz, CDCl₃) δ 11.48 (br s, 1H), 9.10 (s, 2H), 8.67 (d, J=4.4 Hz, 1H), 8.28 (d, J=5.2 Hz, 1H), 7.93-7.85 (m, 1H), 7.79 (d, J=8.0 Hz, 1H), 7.41-7.34 (m, 1H), 7.02 (d, J=5.2 Hz, 1H), 3.91-3.70 (m, 4H), 2.48-2.33 (m, 2H); MS (ES+) m/z 417.1(M+1).

### Step 4. Preparation of 2-(cyclopropylmethoxy)-N-(2′-(3,3-difluoropyrrolidin-1-yl)-[2,4′-bipyridin]-3′-yl)pyrimidine-5-carboxamide

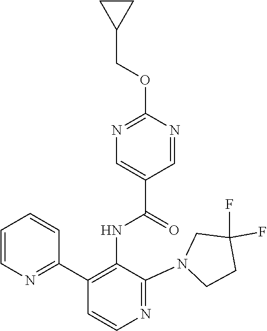 [SMILES: O=C(Nc1c(-c2ccccn2)ccnc1N1CCC(F)(F)C1)c1cnc(OCC2CC2)nc1]

To a solution of 2-chloro-N-(2′-(3,3-difluoropyrrolidin-1-yl)-[2,4′-bipyridin]-3′-yl)pyrimidine-5-carboxamide (0.0300 g, 0.0720 mmol) in tetrahydrofuran (0.25 mL) and dimethylformamide (0.25 mL) was added cesium carbonate (0.0938 g, 0.288 mmol), 1,4-diazabicyclo[2.2.2]octane (0.00161 g, 0.0144 mmol) and cyclopropylmethanol (0.0104 g, 0.144 mmol). The mixture was stirred at 50° C. for 12 h. After cooling to ambient temperature, the mixture was filtered. The residue was purified by prep-HPLC, eluting with a gradient of 28-58% of acetonitrile in water containing 0.225% formic acid, to afford the title compound as a yellow solid (0.0104 g,32% yield): ¹H NMR (400 MHz, CDCl₃) δ 11.09 (br s, 1H), 9.00 (s, 2H), 8.67 (d, J=4.4 Hz, 1H), 8.26 (d, J=5.2 Hz, 1H), 7.86 (dt, J=1.6, 7.6 Hz, 1H), 7.75 (d, J=8.0 Hz, 1H), 7.34 (dd, J=5.2, 7.2 Hz, 1H), 6.99 (d, J=5.2 Hz, 1H), 4.29 (d, J=7.2 Hz, 2H), 3.91-3.72 (m, 4H), 2.40 (tt, J=7.2, 13.6 Hz, 2H), 1.42-1.30 (m, 1H), 0.70-0.59 (m, 2H), 0.47-0.35 (m, 2H); MS (ES+) m/z 453.2(M+1).

### Example 248-249

In a similar manner as described in Example 247, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 248 | 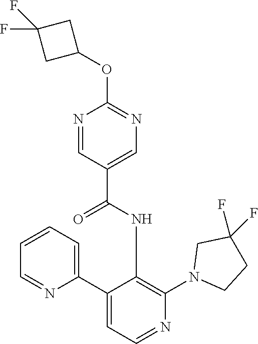 [SMILES: O=C(Nc1c(-c2ccccn2)ccnc1N1CCC(F)(F)C1)c1cnc(OC2CC(F)(F)C2)nc1] 2-(3,3-difluorocyclobutoxy)-N-(2′-(3,3- difluoropyrrolidin-1-yl)-[2,4′- bipyridin]-3′-yl)pyrimidine-5- carboxamide | 33% | 488.2 (M + 1) | (400 MHz; CDCl₃) δ 11.23 (dd, J = 1.0, 0.7 Hz, 1H), 9.03 (s, 2H), 8.69 (dd, J = 4.8, 0.9 Hz, 1H), 8.28 (d, J = 5.0 Hz, 1H), 7.89 (td, J = 7.8, 1.8 Hz, 1H), 7.78 (d, J = 8.0 Hz, 1H), 7.37 (ddd, J = 7.5, 5.0, 0.9 Hz, 1H), 7.02 (d, J = 5.1 Hz, 1H), 5.26-5.20 (m, 1H), 3.87 (q, J = 7.5 Hz, 2H), 3.80-3.77 (m, 2H), 3.25-3.15 (m, 2H), 2.93- 2.81 (m, 2H), 2.47-2.37 (m, 2H). |
| 249 | 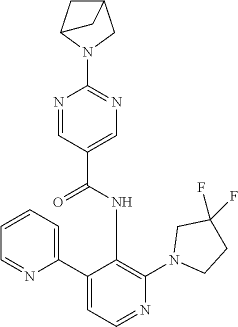 [SMILES: O=C(Nc1c(-c2ccccn2)ccnc1N1CCC(F)(F)C1)c1cnc(N2CC3CC2C3)nc1] 2-(2-azabicyclo[2.1.1] hexan-2-yl)-N-(2′-(3,3- difluoropyrrolidin-1-yl)-[2,4′- bipyridin]-3′-yl)pyrimidine-5- carboxamide | 35% | 464.2 (M + 1) | (400 MHz, CDCl₃) δ 10.70 (s, 1H), 8.78 (s, 2H), 8.69 (dd, J = 0.8, 4.8 Hz, 1H), 8.23 (d, J = 5.2 Hz, 1H), 7.84 (dt, J = 1.6, 7.6 Hz, 1H), 7.71 (d, J = 8.0 Hz, 1H), 7.35-7.29 (m, 1H), 6.95 (d, J = 5.2 Hz, 1H), 5.02 (d, J = 7.2 Hz, 1H), 3.94-3.74 (m, 4H), 3.60 (s, 2H), 3.07-2.95 (m, 1H), 2.39 (tt, J = 7.2, 13.6 Hz, 2H), 2.09 (s, 2H), 1.52 (dd, J = 1.6, 4.4 Hz, 2H). |

### Example 250

### Synthesis of N-(2′-((3S,4R)-3,4-difluoropyrrolidin-1-yl)-3-fluoro-[2,4′-bipyridin]-3′-yl)-5-fluoro-6-methoxynicotinamide

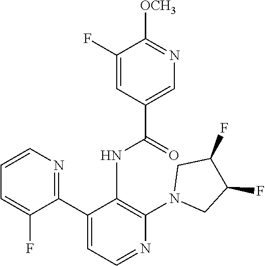 [SMILES: COc1ncc(C(=O)Nc2c(-c3ncccc3F)ccnc2N2C[C@@H](F)[C@@H](F)C2)cc1F]

### Step 1. Preparation of 2-((3S,4R)-3,4-difluoropyrrolidin-1-yl)-4-iodonicotinic acid

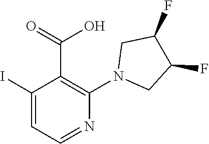 [SMILES: O=C(O)c1c(I)ccnc1N1C[C@@H](F)[C@@H](F)C1]

To the mixture of 2-fluoro-4-iodo-pyridine-3-carboxylic acid (2.20 g, 8.24 mmol) and potassium carbonate (2.28 g, 16.5 mmol) in dimethyl formamide (50 mL) was added (3R,4S)-3,4-difluoropyrrolidine hydrochloride (1.18 g, 8.24 mmol). The mixture was stirred at 85° C. for 12 h. After cooling to ambient temperature the mixture was diluted with ethyl acetate (250 mL). The reaction mixture was filtered and the filter cake was washed with ethyl acetate (30 mL). The filtrate was concentrated in vacuo to afford the title compound as a yellow solid (4.47g, crude):¹H NMR (400 MHz, DMSO-d₆) δ 7.45 (d, J=5.2 Hz, 1H), 6.93 (d, J=5.2 Hz, 1H), 5.45-5.30 (m, 1H), 5.29-5.17 (m, 1H), 4.04-3.87 (m, 2H), 3.80-3.66 (m, 2H).

### Step 2. Preparation of 2-((3S,4R)-3,4-difluoropyrrolidin-1-yl)-4-iodopyridin-3-amine

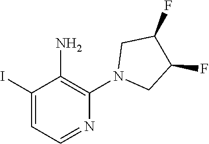 [SMILES: Nc1c(I)ccnc1N1C[C@@H](F)[C@@H](F)C1]

To the mixture of 2-((3S,4R)-3,4-difluoropyrrolidin-1-yl)-4-iodonicotinic acid (3.47 g, 9.80 mmol) and triethylamine (2.48 g, 24.5 mmol) in 1-methylpyrrolidin-2-one (150 mL) was added diphenylphosphoryl azide (4.05 g, 14.7 mmol). The mixture was stirred at 95° C. for 12 h. After cooling to ambient temperature, the mixture was diluted with saturated aqueous sodium bicarbonate (200 ml), and extracted with ethyl acetate (3×200 mL). The combined organic phases were washed with brine (200 mL), dried over anhydrous sodium sulfate, filtered and concentrated in vacuo. The residue was purified by flash chromatography on silica gel, eluting with 10% of ethyl acetate in petroleum ether to afford the title compound as a brown solid (1.11 g, 34% yield): ¹H NMR (400 MHz, DMSO-d₆) δ 7.34 (d, J=5.6 Hz, 1H), 7.21 (d, J=5.6 Hz, 1H), 5.49-5.39 (m, 1H), 5.35-5.26 (m, 1H), 4.03-3.84 (m, 2H), 3.84-3.63 (m, 2H).

### Step 3. Preparation of N-(2-((3S,4R)-3,4-difluoropyrrolidin-1-yl)-4-iodopyridin-3-yl)-5-fluoro-6-methoxynicotinamide

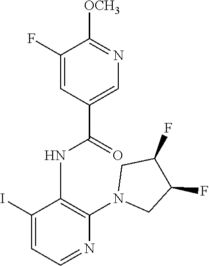 [SMILES: COc1ncc(C(=O)Nc2c(I)ccnc2N2C[C@@H](F)[C@@H](F)C2)cc1F]

To a mixture of 5-fluoro-6-methoxy-pyridine-3-carboxylic acid (0.405 g, 2.37 mmol) and 2-chloro-1-methyl-pyridin-1-ium iodide (0.605 g, 2.37 mmol) in tetrahydrofuran (10 mL) was added N-ethyl-N-isopropylpropan-2-amine (1.11 g, 8.61 mmol) and 2-((3S,4R)-3,4-difluoropyrrolidin-1-yl)-4-iodopyridin-3-amine (0.700 g, 2.15 mmol) in one portion at 20° C. The mixture was stirred at 70° C. for 12 h. The mixture was cooled to 20° C. and poured into water (20 mL). The mixture was extracted with ethyl acetate (3×20 mL). The combined organic layer was washed with brine (20 mL), dried over sodium sulfate, filtered and the filtrate was evaporated under reduced pressure. The residue was purified by flash chromatography on silica gel, eluting with a mixture of 54/46 petroleum ether in ethyl acetate, followed by preparative HPLC, eluting with a gradient of 30-60% of acetonitrile in water containing 0.225% formic acid, to afford the title compound as a colorless solid (0.0660 g, 6% yield); ¹H NMR (400 MHz, DMSO-d₆) δ 10.25 (s, 1H), 8.68 (d, J=2.0 Hz, 1H), 8.16 (dd, J=2.0, 10.8 Hz, 1H), 7.74 (d, J=4.8 Hz, 1H), 7.30 (d, J=5.2 Hz, 1H), 5.44-5.33 (m, 1H), 5.31-5.20 (m, 1H), 4.04 (s, 3H), 3.77 (d, J=4.4 Hz, 1H), 3.72 (d, J=4.0 Hz, 1H), 3.60-3.44 (m, 2H).

### Step 4. Preparation of N-(2′-((3S,4R)-3,4-difluoropyrrolidin-1-yl)-3-fluoro-[2,4′-bipyridin]-3′-yl)-5-fluoro-6-methoxynicotinamide

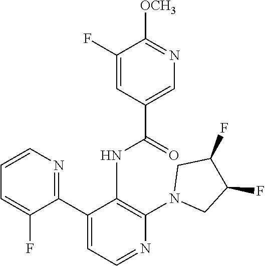 [SMILES: COc1ncc(C(=O)Nc2c(-c3ncccc3F)ccnc2N2C[C@@H](F)[C@@H](F)C2)cc1F]

To a mixture of N-(2-((3S,4R)-3,4-difluoropyrrolidin-1-yl)-4-iodopyridin-3-yl)-5-fluoro-6-methoxynicotin-amide (0.0500 g, 0.105 mmol) and tributyl-(3-fluoro-2-pyridyl)stannane (0.0606 g, 0.157 mmol) in toluene (5 mL) was added tetrakis[triphenylphosphine]palladium(0) (0.0121 g, 0.0105 mmol) and copper(I) iodide (0.00199 g, 0.0105 mmol) in one portion at 20° C. The mixture was stirred at 110° C. under nitrogen atmosphere for 12 h. The mixture was cooled to 20° C. and evaporated under reduced pressure. The residue was diluted with saturated aqueous sodium bicarbonate (10 mL) and ethyl acetate (10 mL). The layers were separated, and the aqueous phase was extracted with ethyl acetate (2×10 mL). The combined organic layers were washed with brine (10 mL), dried over anhydrous sodium sulfate, filtered and the filtrate was evaporated under reduced pressure. The residue was purified by column chromatography on silica gel, eluting with 90-100% of ethyl acetate in petroleum ether, followed by prep-NPLC, eluting with a gradient of 10-50% of ethanol in hexanes. The product was further purified by prep-HPLC, eluting with a gradient of 31-61% of acetonitrile in water containing ammonium bicarbonate to afford the title compound as a colorless solid (0.0247 g, 51% yield): ¹H NMR (400 MHz, DMSO-d₆) δ 5 10.03 (s, 1H), 8.41 (d, J=4.4 Hz, 1H), 8.36 (d, J=1.6 Hz, 1H), 8.22 (d, J=4.8 Hz, 1H), 7.87 (dd, J=1.6, 10.8 Hz, 1H), 7.75 (t, J=9.2 Hz, 1H), 7.43 (td, J=4.4, 8.4 Hz, 1H), 6.87 (d, J=4.8 Hz, 1H), 5.47-5.35 (m, 1H), 5.33-5.21 (m, 1H), 3.98 (s, 3H), 3.95-3.80 (m, 2H), 3.79-3.61 (m, 2H); MS (ES+) m/z 448.1 (M+1).

### Example 251

### Synthesis of N-(2-((3S,4R)-3,4-difluoropyrrolidin-1-yl)-4-(3,4-dihydro-2H-pyran-6-yl)pyridin-3-yl)-5-fluoro-6-methoxynicotinamide

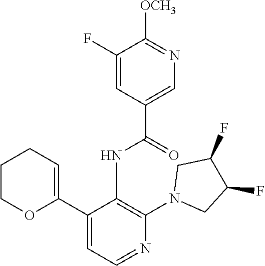 [SMILES: COc1ncc(C(=O)Nc2c(C3=CCCCO3)ccnc2N2C[C@@H](F)[C@@H](F)C2)cc1F]

### Step 1. Preparation of 2-((3S,4R)-3,4-difluoropyrrolidin-1-yl)-4-(3,4-dihydro-2H-pyran-6-yl)pyridin-3-amine

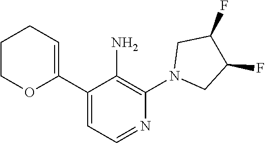 [SMILES: Nc1c(C2=CCCCO2)ccnc1N1C[C@@H](F)[C@@H](F)C1]

To the mixture of 2-((3S,4R)-3,4-difluoropyrrolidin-1-yl)-4-iodopyridin-3-amine (0.200 g, 0.0615 mmol), 2-(3,4-dihydro-2H-pyran-6-yl)-4,4,5,5-tetramethyl-1,3,2-dioxaborolane (0.388 g, 1.85 mmol) and potassium carbonate (0.128 g, 0.923 mmol) in dioxane (7 mL) and water (0.7 mL) was added [1,1′-bis(diphenylphosphino)ferrocene]dichloropalladium(II) (0.0450 g, 0.0615 mmol). The mixture was stirred at 90° C. under nitrogen atmosphere for 12 h. The mixture was cooled to 20° C. and poured into water (20 mL). The mixture was extracted with ethyl acetate (3×20 mL). The combined organic layers were washed with brine (50 mL), dried over sodium sulfate, filtered and the filtrate was evaporated under reduced pressure. The residue was purified by flash chromatography on silica gel, eluting with 18% of ethyl acetate in petroleum ether, to afford the title compound as a brown oil (0.122 g, 70% yield):¹H NMR (400 MHz, CDCl₃) δ 7.65 (d, J=5.2 Hz, 1H), 6.84 (d, J=5.2 Hz, 1H), 5.30-5.22 (m, 1H), 5.22-5.17 (m, 1H), 5.16-5.10 (m, 1H), 4.31 (s, 2H), 4.24-4.17 (m, 2H), 3.81-3.61 (m, 4H), 2.23 (dt, J=4.0, 6.4 Hz, 2H), 1.95 (dd, J=4.8, 5.6 Hz, 2H).

### Step 2. Preparation of N-(2-((3S,4R)-3,4-difluoropyrrolidin-1-yl)-4-(3,4-dihydro-2H-pyran-6-yl)pyridin-3-yl)-5-fluoro-6-methoxynicotinamide

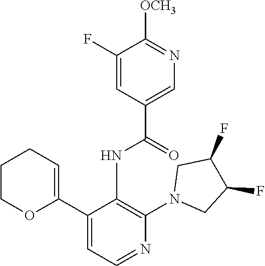 [SMILES: COc1ncc(C(=O)Nc2c(C3=CCCCO3)ccnc2N2C[C@@H](F)[C@@H](F)C2)cc1F]

To the mixture of 5-fluoro-6-methoxy-pyridine-3-carboxylic acid (0.0803 g, 0.469 mmol) and 2-chloro-1-methyl-pyridin-1-ium iodide (0.120 g, 0.469 mmol) in tetrahydrofuran (4 mL) were added diisopropylethylamine (0.221 g, 1.71 mmol) and 2-((3S,4R)-3,4-difluoropyrrolidin-1-yl)-4-(3,4-dihydro-2H-pyran-6-yl)pyridin-3-amine (0.120 g, 0.427 mmol) in one portion. The mixture was stirred at 70° C. for 12 h. The mixture was cooled to 20° C. and poured into water (20 mL). The mixture was extracted with ethyl acetate (3×20 mL). The combined organic layer was washed with brine (20 mL), dried over sodium sulfate, filtered and the filtrate was evaporated under reduced pressure. The residue was purified by flash chromatography on silica gel, eluting with 30% of ethyl acetate in petroleum ether, followed by prep-HPLC, eluting with a gradient of 38-68% acetonitrile in water containing ammonium bicarbonate. The product was purified by prep-NPLC, eluting with a gradient of 15-55% of ethanol in hexanes, followed by prep-HPLC, eluting with a gradient of 38-68% of acetonitrile in water containing ammonium bicarbonate to afford the title compound as a colorless solid (20.6 mg, 12% yield): ¹H NMR (400 MHz, DMSO-d₆) δ 9.82 (s, 1H), 8.63 (d, J=1.6 Hz, 1H), 8.12 (dd, J=1.6, 10.8 Hz, 1H), 8.06 (d, J=5.2 Hz, 1H), 6.73 (d, J=4.8 Hz, 1H), 5.43-5.32 (m, 1H), 5.24 (dd, J=3.6, 8.8 Hz, 1H), 5.04 (t, J=3.6 Hz, 1H), 4.03 (s, 3H), 3.98-3.54 (m, 6H), 2.05-1.97 (m, 2H), 1.76-1.63 (m, 2H); MS (ES+) m/z 435.1 (M+1).

### Example 252

### Synthesis of N-(2-(2,2-dimethylpyrrolidin-1-yl)-4-(o-tolyl)pyridin-3-yl)-2-isopropylpyrimidine-5-carboxamide

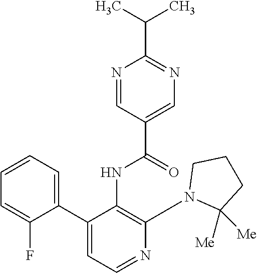 [SMILES: CC(C)c1ncc(C(=O)Nc2c(-c3ccccc3F)ccnc2N2CCCC2(C)C)cn1]

### Step 1. Preparation of 2-(2,2-dimethylpyrrolidin-1-yl)-4-(2-fluorophenyl)-3-nitropyridine

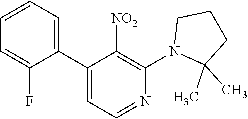 [SMILES: CC1(C)CCCN1c1nccc(-c2ccccc2F)c1[N+](=O)[O-]]

To a mixture of 2-chloro-4-(2-fluorophenyl)-3-nitropyridine (0.200 g, 0.792 mmol) in dimethylsulfoxide (3 mL) were added N,N-diisopropylethylamine (0.306 g, 2.38 mmol) and 2,2-dimethylpyrrolidine (0.0942 g, 0.950 mmol). The mixture was stirred at 120° C. for 12 h. The reaction mixture was cooled to ambient temperature, poured into brine (20 mL), and extracted with ethyl acetate (3×15 mL). The combined organic extracts were washed with brine (30 mL) and dried over anhydrous sodium sulfate, filtered and the filtrate was concentrated in vacuo. The residue was purified by column chromatography on silica gel, eluting with 33 # of ethyl acetate in petroleum ether to afford the title compound as a yellow oil (0.230 g, 61% yield): ¹H NMR (400 MHz,CDCl₃) δ 8.24 (d, J=4.8 Hz, 1H), 7.43-7.35 (m, 1H), 7.25-7.10 (m, 3H), 6.47 (d, J=4.8 Hz, 1H), 3.18 (t, J=6.4 Hz, 2H), 1.98-1.83 (m, 4H), 1.67 (s, 6H).

### Step 2. Preparation of 2-(2,2-dimethylpyrrolidin-1-yl)-4-(2-fluorophenyl)pyridin-3-amine

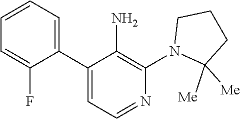 [SMILES: CC1(C)CCCN1c1nccc(-c2ccccc2F)c1N]

To a mixture of 2-(2,2-dimethylpyrrolidin-1-yl)-4-(2-fluorophenyl)-3-nitropyridine (0.200 g, 0.634 mmol) in methanol (3 mL) was added palladium on activated carbon (0.200 g, 10 wt %) under a nitrogen atmosphere. The mixture was stirred at 25° C. under a hydrogen atmosphere (15 psi, balloon) for 12 h. The reaction mixture was filtered over diatomaceous earth (i.e., Celite®) and the filtrate was concentrated under reduced pressure to afford the title compound as a black-brown oil (0.150 g, 83% yield): ¹H NMR (400 MHz, DMSO-d₆) δ 7.67 (d, J=4.8 Hz, 1H), 7.51-7.37 (m, 2H), 7.36-7.26 (m, 2H), 6.74 (d, J=4.8 Hz, 1H), 4.46 (s, 2H), 3.35 (t, 2H), 1.90 (m, J=7.2 Hz, 2H), 1.77-1.68 (m, 2H), 1.22 (s, 6H).

### Step 3. Preparation of N-(2-(2,2-dimethylpyrrolidin-1-yl)-4-(o-tolyl)pyridin-3-yl)-2-isopropylpyrimidine-5-carboxamide

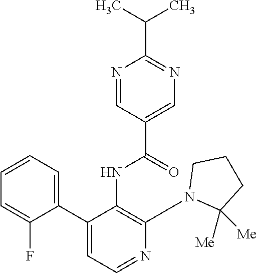 [SMILES: CC(C)c1ncc(C(=O)Nc2c(-c3ccccc3F)ccnc2N2CCCC2(C)C)cn1]

To a mixture of 2-(2,2-dimethylpyrrolidin-1-yl)-4-(2-fluorophenyl)pyridin-3-amine (0.0500 g, 0.175 mmol), 2-isopropylpyrimidine-5-carboxylic acid (0.0349 g, 0.210 mmol) and 2-chloro-1-methyl-pyridin-1-ium iodide (0.0537 g, 0.21 mmol) in tetrahydrofuran (2 mL) was added N,N-diisopropylethylamine (0.0679 g, 0.525 mmol). 15 The mixture was stirred at 70° C. for 12 h. After cooling to ambient temperature, the reaction mixture was concentrated under reduced pressure. The residue was poured into water (20 mL) and extracted with ethyl acetate (3×15 mL). The combined organic extracts were dried over anhydrous sodium sulfate, filtered and the filtrate was concentrated in vacuo. The residue was purified by prep-HPLC, eluting with a gradient 20 of 43-79% of acetonitrile in water containing 0.1% of formic acid, to afford the title compound as a colorless solid (0.0228 g, 29% yield): ¹H NMR (400 MHz, DMSO-d₆) δ 5 9.96 (s, 1H), 8.81 (s, 2H), 8.11 (d, J=4.8 Hz, 1H), 7.38-7.09 (m, 4H), 6.57 (d, J=4.8 Hz, 1H), 3.49 (s, 2H), 3.20-3.10 (m, 1H), 1.83-1.74 (m, 2H), 1.73-1.68 (m, 2H), 1.61-1.54 (m, 6H), 1.25 (d, J=6.8 Hz, 6H); MS (ES+) m/z 434.3 (M+1)

### Example 253

In a similar manner as described in Example 252, utilizing the appropriately substituted starting materials and intermediates, the following compounds were prepared:

| Example | Structure | | MS (ES+) | |
|---|---|---|---|---|
| No. | Name | Yield | m/z | ¹H-NMR |
| 253 | 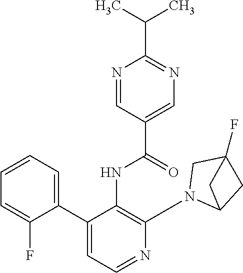 [SMILES: CC(C)c1ncc(C(=O)Nc2c(-c3ccccc3F)ccnc2N2CC3(F)CC2C3)cn1] N-(2-(4-fluoro-2-azabicyclo[2.1.1] hexan-2-yl)-4-(2- fluorophenyl)pyridin-3-yl)-2- isopropylpyrimidine-5-carboxamide | 9% | 436.2 (M + 1) | (400 MHz, MeOD-d₄) δ 8.91 (s, 2H), 8.21 (d, J = 4.8 Hz, 1H), 7.42- 7.30 (m, 2H), 7.24- 7.10 (m, 2H), 6.88 (d, J = 4.8 Hz, 1H), 4.75- 4.63 (m, 1H), 3.33 (s, 2H), 3.23 (td, J = 6.8, 13.6 Hz, 1H), 2.13 (s, 2H), 2.02-1.97 (m, 2H), 1.33 (d, J = 7.2 Hz, 6H) |

### Example 254

### Synthesis of N-(4-(3,3-difluoropyrrolidin-1-yl)-6-(3-fluoropyridin-2-yl)pyrimidin-5-yl)-5-fluoro-6-methoxynicotinamide

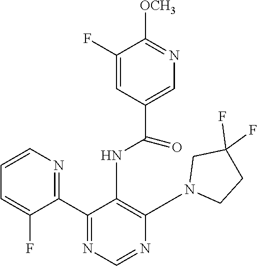 [SMILES: COc1ncc(C(=O)Nc2c(-c3ncccc3F)ncnc2N2CCC(F)(F)C2)cc1F]

### Step 1. Preparation of 4-chloro-6-(3-fluoropyridin-2-yl)pyrimidin-5-amine

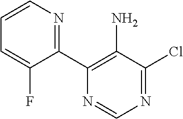 [SMILES: Nc1c(Cl)ncnc1-c1ncccc1F]

To a solution of 4,6-dichloropyrimidin-5-amine (0.450 g, 2.74 mmol) and 3-fluoro-2-(tributylstannyl)pyridine (1.00 g, 2.59 mmol) in toluene (15 mL) were added tetrakis(triphenylphosphine)palladium(0) (0.317 g, 0.274 mmol) and cuprous iodide (0.0260 g, 0.136 mmol) under nitrogen. The mixture was stirred at 120° C. for 3 h under microwave irradiation. The mixture was cooled to 25° C. Potassium fluoride (1.00 g) was added to the mixture. The reaction mixture was diluted with water (20 mL) and extracted with ethyl acetate (2×20 mL). The combined organic layers were washed with brine (20 mL), dried over sodium sulfate, filtered and concentrated under reduced pressure. The residue was purified by prep-HPLC, eluting with a gradient of 12-42% of acetonitrile in water containing 0.225% formic acid, to afford the title compound as a yellow solid (0.100 g, 15% yield): ¹H NMR (400 MHz, DMSO-d₆) 58.59 (s, 1H), 8.35 (s, 1H), 7.99-7.88 (m, 1H), 7.66 (dd, J=4.4, 8.4 Hz, 1H), 6.45 (s, 2H); MS (ES+) m/z 225.1, 227.1 (M+1).

### Step 2. Preparation of 4-(3,3-difluoropyrrolidin-1-yl)-6-(3-fluoropyridin-2-yl)pyrimidin-5-amine

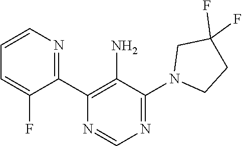 [SMILES: Nc1c(-c2ncccc2F)ncnc1N1CCC(F)(F)C1]

A solution of 4-chloro-6-(3-fluoropyridin-2-yl)pyrimidin-5-amine (0.100 g, 0.445 mmol), 3,3-difluoropyrrolidine hydrochloride (0.128 g, 0.891 mmol) and N,N-diisopropylethylamine (0.288 g, 2.23 mmol) in dimethylsulfoxide (3 mL) was stirred at 120° C. for 12 h. The reaction mixture was cooled to 25° C. The reaction mixture was diluted with water (10 mL) and extracted with ethyl acetate (2×15 mL). The combined organic layers were washed with brine (10 mL), dried over sodium sulfate, filtered and concentrated under reduced pressure. The residue was purified by flash silica gel chromatography, eluting with a gradient of 60-70% of ethyl acetate in petroleum ether, to afford the title compound as a yellow oil (0.100 g, 68% yield) as yellow oil: ¹H NMR (400 MHz, DMSO-d₆) δ 8.53 (td, J=1.6, 4.4 Hz, 1H), 8.16 (s, 1H), 7.87 (ddd, J=1.2, 8.4, 10.8 Hz, 1H), 7.58 (td, J=4.0, 8.4 Hz, 1H), 5.68-4.97 (m, 2H), 3.99 (t, J=13.6 Hz, 2H), 3.80 (t, J=7.2 Hz, 2H), 2.48-2.40 (m, 2H); MS (ES+) m/z 296.1 (M+1).

### Step 3. Preparation of N-(4-(3,3-difluoropyrrolidin-1-yl)-6-(3-fluoropyridin-2-yl)pyrimidin-5-yl)-5-fluoro-6-methoxynicotinamide

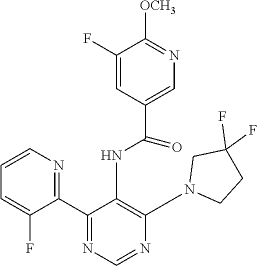 [SMILES: COc1ncc(C(=O)Nc2c(-c3ncccc3F)ncnc2N2CCC(F)(F)C2)cc1F]

A solution of 4-(3,3-difluoropyrrolidin-1-yl)-6-(3-fluoropyridin-2-yl)pyrimidin-5-amine (0.0500 g, 0.169 mmol), 5-fluoro-6-methoxynicotinic acid (0.0320 g, 0.187 mmol), 2-chloro-1-methylpyridinium iodide (0.0650 g, 0.254 mmol) and N,N-diisopropylethylamine (0.0660 g, 0.510 mmol) in tetrahydrofuran (1 mL) was stirred at 70° C. for 12 h. The mixture was cooled to 25° C. The mixture was concentrated under reduced pressure. The residue was purified by prep-HPLC, eluting with a gradient of 25-55% of acetonitrile in water containing 0.225% formic acid, to afford the title compound as a brown solid (0.0137 g, 17% yield): ¹H NMR (400 MHz, MeOD) δ8.61 (s, 1H), 8.44 (d, J=3.2 Hz, 1H), 8.29 (d, J=2.0 Hz, 1H), 7.76-7.65 (m, 2H), 7.49 (td, J=4.0, 8.4 Hz, 1H), 4.07 (s, 1H), 4.05 (s, 3H), 4.00 (d, J=16.8 Hz, 3H), 2.52-2.40 (m, 2H); MS (ES+) m/z 449.1 (M+1).

### Example 255

### Synthesis of N-(4-(3,3-difluoropyrrolidin-1-yl)-6-(3-fluoropyridin-2-yl)pyrimidin-5-yl)-5-fluoro-6-methoxynicotinamide

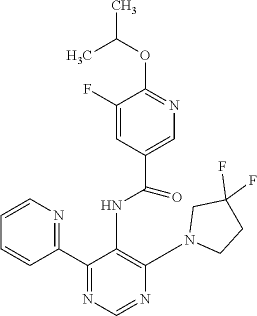 [SMILES: CC(C)Oc1ncc(C(=O)Nc2c(-c3ccccn3)ncnc2N2CCC(F)(F)C2)cc1F]

In a similar manner as described in Example 246, utilizing the appropriately substituted starting materials and intermediates, the title compound was prepared as a purple solid (0.0257 g, 41% yield,): ¹H NMR (400 MHz, DMSO-d₆) δ 10.39 (s, 1H), 8.61 (s, 1H), 8.55 (d, J=4.4 Hz, 1H), 8.47 (d, J=2.0 Hz, 1H), 7.98 (dd, J=2.0, 11.2 Hz, 1H), 7.95-7.89 (m, 2H), 7.45-7.38 (m, 1H), 5.40 (td, J=6.0, 12.4 Hz, 1H), 4.20-4.06 (m, 1H), 4.05-3.84 (m, 2H), 3.81-3.68 (m, 1H), 2.48-2.40 (m, 2H), 1.35 (d, J=6.0 Hz, 6H).

### Example 256

### Synthesis of N-(5-(3,3-difluoropyrrolidin-1-yl)-7-(2-fluorophenyl)-3-methyl-[1,2,4]triazolo[4,3-a]pyridin-6-yl)-2-isopropylpyrimidine-5-carboxamide

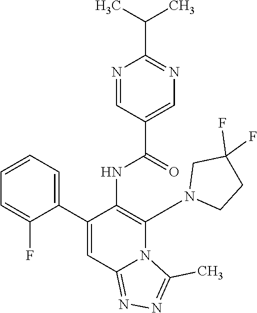 [SMILES: Cc1nnc2cc(-c3ccccc3F)c(NC(=O)c3cnc(C(C)C)nc3)c(N3CCC(F)(F)C3)n12]

### Step 1. Preparation of 3-methyl-6-nitro-[1,2,4]triazolo[4,3-a]pyridine

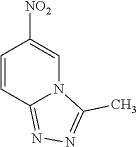

A mixture of (5-nitro-2-pyridyl)hydrazine (1.0 g, 6.5 mmol) and ethanol (25 mL) was charged with trimethylorthopropionate (11 mL). The reaction mixture was heated to reflux for 1 h. After cooling to ambient temperature, the reaction mixture was concentrated in vacuo and the resulting solid was used as is in the next reaction (1.16 g, 99% yield): ¹H-NMR (300 MHz; CDCl₃): δ 9.04 (dd, J=2.0, 1.0 Hz, 1H), 8.02 (dd, J=10.1, 2.0 Hz, 1H), 7.85 (dd, J=10.1, 0.9 Hz, 1H), 2.90 (s, 3H).

### Step 2. Preparation of 3-methyl-[1,2,4]triazolo[4,3-a]pyridin-6-amine

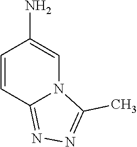

A mixture of 3-methyl-6-nitro-[1,2,4]triazolo[4,3-a]pyridine (1.2 g, 6.5 mmol) and methanol (13 mL) was sparged with nitrogen for 5 min. The reaction mixture was charged with palladium on carbon (0.11 g, 1.1 mmol) and ammonium formate (8.2 g, 130 mmol). The reaction mixture was heated to reflux for 1 h. The reaction mixture was charged with palladium on carbon (0.11 g, 1.1 mmol) and ammonium formate (8.2 g, 130 mmol) and left for a further 3 h. After cooling to ambient temperature, the reaction mixture was diluted with ethyl acetate (100 mL), filtered, and concentrated in vacuo. Purification of the residue by column chromatography, eluting with 0 to 50% methanol in ethyl acetate, afforded the title compound as a green solid (0.48 g, 50% yield): ¹H-NMR (300 MHz; CDCl₃): 57.97 (dd, J=2.2, 0.8 Hz, 1H), 7.48 (dd, J=9.4, 0.8 Hz, 1H), 7.06 (dd, J=9.4, 2.2 Hz, 1H), 3.50 (s, 2H), 2.56 (s, 3H).

### Step 3. Preparation of 5,7-dibromo-3-methyl-[1,2,4]triazolo[4,3-a]pyridin-6-amine

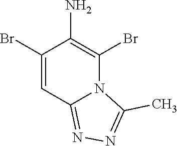

A mixture of 3-methyl-[1,2,4]triazolo[4,3-a]pyridin-6-amine (0.48 g, 3.3 mmol), sodium bicarbonate (0.96 g, 11 mmol), and anhydrous dichloromethane (11 mL) was cooled in an ice/water bath. Bromine (1.6 g, 9.8 mmol) was added via an addition funnel, the reaction mixture was warmed to ambient temperature and stirred for 3 h. The reaction mixture was diluted with ethyl acetate (200 mL), washed with saturated sodium thiosulfate (2×50 mL), dried over anhydrous magnesium sulfate, filtered, and concentrated in vacuo. Purification of the residue by column chromatography, eluting with 5 to 100% ethyl acetate in heptane, afforded the title compound as a brown solid (0.55 g, 55% yield): ¹H-NMR (300 MHz; CDCl₃): δ 7.85 (s, 1H), 4.36 (s, 2H), 2.62 (s, 3H).

### Step 4. Preparation of 5-bromo-7-(2-fluorophenyl)-3-methyl-[1,2,4]triazolo[4,3-a]pyridin-6-amine

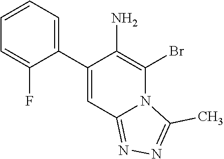 [SMILES: Cc1nnc2cc(-c3ccccc3F)c(N)c(Br)n12]

A mixture of 5,7-dibromo-3-methyl-[1,2,4]triazolo[4,3-a]pyridin-6-amine (0.55 g, 1.8 mmol), 1,4-dioxane (6.0 mL), and water (1.8 mL) was sparged with nitrogen for 10 min. The reaction mixture was charged with 2-fluorophenylboronic acid (0.33 g, 2.3 mmol), potassium carbonate (0.50 g, 3.6 mmol), and [1,1′-bis(diphenylphosphino)ferrocene]dichloropalladium(II), complex with dichloromethane (0.15 g, 0.18 mmol). The reaction mixture was sparged with nitrogen for 2 minutes then stirred at 80° C. for 90 min. After cooling to ambient temperature, the reaction mixture was diluted with ethyl acetate (200 mL) and washed with saturated ammonium chloride solution (2×50 mL). The organic layer was dried over anhydrous magnesium sulfate, filtered, and concentrated in vacuo. The residue was purified by column chromatography, eluting with 5 to 100% ethyl acetate in heptane, afforded the title compound as a colorless solid (0.35 g, 61% yield): MS (ES+) m/z 321.0 (M+1), 323.0 (M+1).

### Step 5. Preparation of 5-(3,3-difluoropyrrolidin-1-yl)-7-(2-fluorophenyl)-3-methyl-[1,2,4]triazolo[4,3-a]pyridin-6-amine

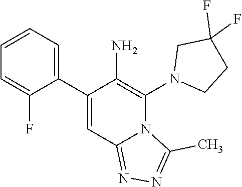 [SMILES: Cc1nnc2cc(-c3ccccc3F)c(N)c(N3CCC(F)(F)C3)n12]

A mixture of 5-bromo-7-(2-fluorophenyl)-3-methyl-[1,2,4]triazolo[4,3-a]pyridin-6-amine (0.15 g, 0.47 mmol) and 1,2-dimethoxyethane (4.7 mL) was sparged with nitrogen for 5 min. The vial was charged with palladium acetate (0.010 g, 0.047 mmol), cyclopenta-2,4-dien-1-yl-[(1R)-2-[(1S)-1-ditert-butylphosphanylethyl]-3-dicyclohexylphosphanyl-cyclopenta-2,4-dien-1-yl]iron (0.026 g, 0.047 mmol), 3,3-difluoropyrrolidine hydrochloride (0.20 g, 1.4 mmol), and a 1.3 M solution of lithium hexamethyldisilazide in tetrahydrofuran (2.2 mL, 2.8 mmol). The vial was sealed and heated to 90° C. for 90 min. After cooling to ambient temperature, the reaction mixture was diluted with ethyl acetate (150 mL). The organic layer was washed with saturated ammonium chloride (2×50 mL), dried over anhydrous magnesium sulfate, filtered, and concentrated in vacuo. Purification of the residue by column chromatography, eluting with 5 to 75% ethyl acetate in heptane, afforded the title compound as a yellow solid (0.10 g, 62% yield): MS (ES+) m/z 348.2 (M+1).

### Step 6. Preparation of N-(5-(3,3-difluoropyrrolidin-1-yl)-7-(2-fluorophenyl)-3-methyl-[1,2,4]triazolo[4,3-a]pyridin-6-yl)-2-isopropylpyrimidine-5-carboxamide

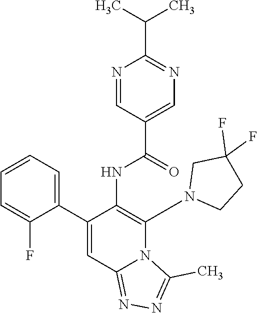 [SMILES: Cc1nnc2cc(-c3ccccc3F)c(NC(=O)c3cnc(C(C)C)nc3)c(N3CCC(F)(F)C3)n12]

To a mixture of 5-(3,3-difluoropyrrolidin-1-yl)-7-(2-fluorophenyl)-3-methyl-[1,2,4]triazolo[4,3-a]pyridin-6-amine (0.10 g, 0.29 mmol), 2-chloro-1-methylpyridinium iodide (0.18 g, 0.73 mmol), 2-isopropylpyrimidine-5-carboxylic acid (0.053 g, 0.32 mmol), and anhydrous tetrahydrofuran (3 mL) was added N,N-diisopropylethylamine (0.38 g, 2.9 mmol). The reaction vessel was sealed and heated to 60° C. for 1 h. After cooling to ambient temperature, the reaction mixture was diluted with ethyl acetate (200 mL). The organic layer was washed with 1M sodium hydroxide (50 mL) and saturated ammonium chloride (2×50 mL), dried over anhydrous magnesium sulfate, filtered, and concentrated in vacuo. The residue was purified by column chromatography, eluting with a gradient of 8 to 100% of ethyl acetate in heptane, to provide the title compound as a colorless solid (0.062 g, 42% yield): ¹H-NMR (300 MHz; CDCl₃): δ 10.24 (s, 1H), 8.86 (s, 2H), 7.46-7.45 (m, 1H), 7.44-7.22 (m, 4H), 4.13 (t, J=13.5 Hz, 2H), 3.83 (t, J=7.0 Hz, 2H), 3.18 (quintet, J=6.9 Hz, 1H), 2.54-2.52 (m, 3H), 2.49-2.41 (m, 2H), 1.28-1.26 (m, 6H); MS (ES+) m/z 496.2 (M+1).

### Example 257

### Synthesis of N-(2-(3,3-difluoropyrrolidin-1-yl)-4-(2-fluorophenyl)pyridin-3-yl)-6-(tetrahydrofuran-2-yl)nicotinamide

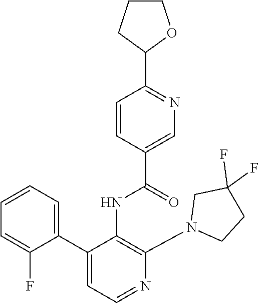 [SMILES: O=C(Nc1c(-c2ccccc2F)ccnc1N1CCC(F)(F)C1)c1ccc(C2CCCO2)nc1]

### Step 1. Preparation of 6-chloro-N-(2-(3,3-difluoropyrrolidin-1-yl)-4-(2-fluorophenyl)pyridin-3-yl)nicotinamide

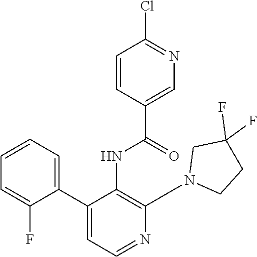 [SMILES: O=C(Nc1c(-c2ccccc2F)ccnc1N1CCC(F)(F)C1)c1ccc(Cl)nc1]

To a mixture of 2-(3,3-difluoropyrrolidin-1-yl)-4-(2-fluorophenyl)pyridin-3-amine hydrochloride (0.50 g, 1.5 mmol), 2-chloro-1-methylpyridinium iodide (0.93 g, 3.6 mmol), 6-chloronicotinic acid (0.29 g, 1.8 mmol), and anhydrous tetrahydrofuran (25 mL) was added N,N-diisopropylethylamine (2.0 g, 15 mmol). The reaction vessel was sealed and heated to 60° C. for 4 h. After cooling to ambient temperature, the reaction mixture was diluted with ethyl acetate (150 mL). The organic layer was washed with 1M sodium hydroxide (50 mL), saturated ammonium chloride (2×50 mL), dried over anhydrous magnesium sulfate, filtered, and concentrated in vacuo. The residue was purified by column chromatography, eluting with a gradient of 10 to 70% of ethyl acetate in heptane, to provide the title compound as a colorless solid (0.54 g, 82% yield): MS (ES+) m/z 433.0 (M+1), 435.2 (M+1).

### Step 2. Preparation of N-(2-(3,3-difluoropyrrolidin-1-yl)-4-(2-fluorophenyl)pyridin-3-yl)-6-(tetrahydrofuran-2-yl)nicotinamide

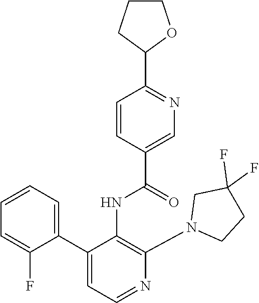 [SMILES: O=C(Nc1c(-c2ccccc2F)ccnc1N1CCC(F)(F)C1)c1ccc(C2CCCO2)nc1]

A mixture of 6-chloro-N-(2-(3,3-difluoropyrrolidin-1-yl)-4-(2-fluorophenyl)pyridin-3-yl)nicotinamide (0.10 g, 0.23 mmol), cesium carbonate (0.14 g, 0.42 mmol), (R/S)-2-tetrahydrofuroic acid (0.046 g, 0.39 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0026 g, 0.0023 mmol), dichloro(dimethoxyethane)nickel (0.0051 g, 0.023 mmol), and 4-tert-butyl-2-(4-tert-butyl-2-pyridyl)pyridine (0.0093 g, 0.035 mmol) was dissolved in N,N-dimethylformamide (3.8 mL). The headspace in the vial was sparged with nitrogen, the vial was sealed, and the reaction mixture was stirred in front of Kessil PR160L lights (440 nm) for 18 h. The reaction mixture was diluted with ethyl acetate (100 mL). The organic layer was washed with saturated ammonium chloride (2×50 mL), dried over anhydrous magnesium sulfate, filtered, and concentrated in vacuo. The residue was purified by column chromatography, eluting with a gradient of 5 to 75% of ethyl acetate in heptane, to provide the title compound as a colorless solid (0.020 g, 17% yield): ¹H-NMR (400 MHz; DMSO-d₆) δ 10.03 (s, 1H), 8.71 (d, J=2.1 Hz, 1H), 8.20 (d, J=5.0 Hz, 1H), 7.99 (dd, J=8.2, 2.2 Hz, 1H), 7.49-7.46 (m, 1H), 7.38-7.27 (m, 2H), 7.26-7.22 (m, 1H), 7.19-7.15 (m, 1H), 6.81 (d, J=4.9 Hz, 1H), 4.91 (dd, J=7.3, 6.1 Hz, 1H), 4.01-3.95 (m, 1H), 3.95-3.82 (m, 3H), 3.82-3.70 (m, 2H), 2.48-2.38 (m, 2H), 2.37-2.28 (m, 1H), 1.95-1.81 (m, 3H); MS (ES+) m/z 469.2 (M+1).

### Example 258

### Synthesis of N-(2-(3,3-difluoropyrrolidin-1-yl)-4-(2-fluorophenyl)pyridin-3-yl)-6-(1-methoxyethyl)nicotinamide

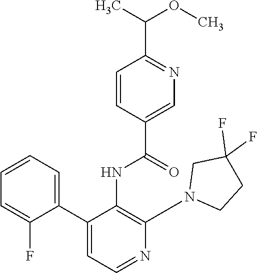 [SMILES: COC(C)c1ccc(C(=O)Nc2c(-c3ccccc3F)ccnc2N2CCC(F)(F)C2)cn1]

A mixture of 6-chloro-N-(2-(3,3-difluoropyrrolidin-1-yl)-4-(2-fluorophenyl)pyridin-3-yl)nicotinamide (0.10 g, 0.23 mmol), cesium carbonate (0.14 g, 0.42 mmol), 2-methoxypropanoic acid (0.041 g, 0.39 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0026 g, 0.0023 mmol), dichloro(dimethoxyethane)nickel (0.0051 g, 0.023 mmol), and 4-tert-butyl-2-(4-tert-butyl-2-pyridyl)pyridine (0.0093 g, 0.035 mmol) was dissolved in N,N-dimethylformamide (3.8 mL). The headspace in the vial was sparged with nitrogen, the vial was sealed, and the reaction mixture was stirred in front of Kessil PR160L lights (440 nm) for 18 h. The reaction mixture was diluted with ethyl acetate (100 mL). The organic layer was washed with saturated ammonium chloride (2×50 mL), dried over anhydrous magnesium sulfate, filtered, and concentrated in vacuo. The residue was purified by column chromatography, eluting with a gradient of 10 to 60% of ethyl acetate in heptane, to provide the title compound as a colorless solid (0.013 g, 12% yield): ¹H-NMR (400 MHz; DMSO-d₆) δ 10.04 (s, 1H), 8.71 (dd, J=2.2, 0.7 Hz, 1H), 8.20 (d, J=5.0 Hz, 1H), 8.02 (dd, J=8.2, 2.3 Hz, 1H), 7.47 (d, J=8.2 Hz, 1H), 7.39-7.29 (m, 2H), 7.24 (ddd, J=10.0, 8.6, 1.2 Hz, 1H), 7.18 (td, J=7.5, 1.1 Hz, 1H), 6.81 (d, J=4.9 Hz, 1H), 4.40 (q, J=6.5 Hz, 1H), 3.93-3.86 (m, 2H), 3.76-3.73 (m, 2H), 3.21 (s, 3H), 2.43 (td, J=14.2, 7.2 Hz, 2H), 1.35 (d, J=6.5 Hz, 3H); MS (ES+) m/z 457.2 (M+1).

### Example 259

### Synthesis of N-(2-(3,3-difluoropyrrolidin-1-yl)-4-(2-fluorophenyl)pyridin-3-yl)-2-(tetrahydrofuran-2-yl)pyrimidine-5-carboxamide

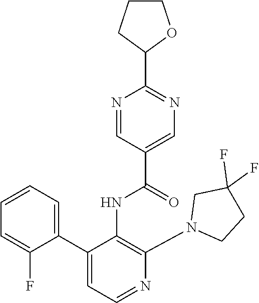 [SMILES: O=C(Nc1c(-c2ccccc2F)ccnc1N1CCC(F)(F)C1)c1cnc(C2CCCO2)nc1]

To a mixture of 2-(3,3-difluoropyrrolidin-1-yl)-4-(2-fluorophenyl)pyridin-3-amine hydrochloride (0.25 g, 0.77 mmol), 2-chloro-1-methylpyridinium iodide (0.59 g, 2.3 mmol), 2-(tetrahydrofuran-2-yl)pyrimidine-5-carboxylic acid (0.15 g, 0.77 mmol), and anhydrous tetrahydrofuran (15 mL) was added N,N-diisopropylethylamine (1.0 g, 7.7 mmol). The reaction vessel was sealed and heated to 60° C. for 90 min. After cooling to ambient temperature, the reaction mixture was diluted with ethyl acetate (200 mL). The organic layer was washed with saturated ammonium chloride (2×50 mL), dried over anhydrous magnesium sulfate, filtered, and concentrated in vacuo. The residue was purified by column chromatography, eluting with a gradient of 10 to 100% of ethyl acetate in heptane, to provide the title compound as a purple solid (0.16 g, 44% yield): ¹H-NMR (400 MHz; DMSO-d₆) δ 10.24 (s, 1H), 8.91 (q, J=2.7 Hz, 2H), 8.22 (d, J=5.0 Hz, 1H), 7.40-7.34 (m, 1H), 7.34-7.29 (m, 1H), 7.29-7.23 (m, 1H), 7.19 (td, J=7.5, 1.1 Hz, 1H), 6.83-6.82 (m, 1H), 5.01-4.98 (m, 1H), 4.01-3.84 (m, 4H), 3.83-3.72 (m, 2H), 2.50-2.39 (m, 2H), 2.33-2.26 (m, 1H), 2.06-1.99 (m, 2H), 1.96-1.89 (m, 1H); MS (ES+) m/z: 470.0 (M+1).

### Example 260

### Synthesis of N-(2-(3,3-difluoropyrrolidin-1-yl)-4-(2-fluorophenyl)pyridin-3-yl)-2-(isoxazolidin-2-yl)pyrimidine-5-carboxamide

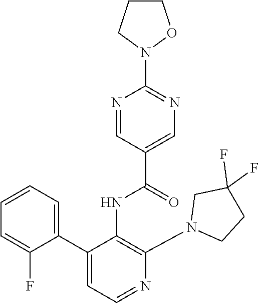 [SMILES: O=C(Nc1c(-c2ccccc2F)ccnc1N1CCC(F)(F)C1)c1cnc(N2CCCO2)nc1]

To a mixture of 2-chloro-N-[2-(3,3-difluoropyrrolidin-1-yl)-4-(2-fluorophenyl)-3-pyridyl]pyrimidine-5-carboxamide (0.80 g, 0.18 mmol), isoxazolidine hydrochloride (0.040 g, 0.37 mmol), and anhydrous N,N-dimethylformamide (1.8 mL) was added anhydrous potassium carbonate (0.10 g, 0.74 mmol). The reaction vessel was sealed and stirred at ambient temperature overnight. The reaction mixture was diluted with ethyl acetate (125 mL). The organic layer was washed with saturated ammonium chloride (2×50 mL), dried over anhydrous magnesium sulfate, filtered, and concentrated in vacuo. The residue was purified by column chromatography, eluting with a gradient of 15 to 100% of ethyl acetate in heptane, to provide the title compound as a colorless solid (0.066 g, 74% yield): ¹H-NMR (400 MHz; DMSO-d₆) δ 9.89 (s, 1H), 8.69-8.67 (m, 2H), 8.19 (d, J=5.0 Hz, 1H), 7.39-7.33 (m, 1H), 7.31-7.27 (m, 1H), 7.24 (ddd, J=10.0, 8.6, 1.2 Hz, 1H), 7.18 (td, J=7.5, 1.1 Hz, 1H), 6.80 (d, J=5.0 Hz, 1H), 3.95-3.92 (m, 2H), 3.92-3.86 (m, 2H), 3.85-3.81 (m, 2H), 3.80-3.70 (m, 2H), 2.50-2.38 (m, 3H), 2.30-2.23 (m, 2H); MS (ES+) m/z 471.2 (M+1).

### Example 261

### Synthesis of (5-((2-(3,3-difluoropyrrolidin-1-yl)-4-(2-fluorophenyl)pyridin-3-yl)carbamoyl)pyrimidin-2-yl)-L-valine

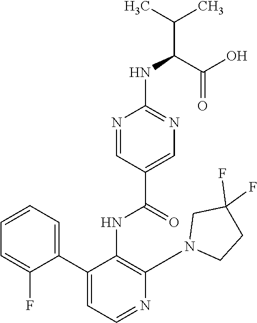 [SMILES: CC(C)[C@H](Nc1ncc(C(=O)Nc2c(-c3ccccc3F)ccnc2N2CCC(F)(F)C2)cn1)C(=O)O]

### Step 1. Preparation of tert-butyl (5-((2-(3,3-difluoropyrrolidin-1-yl)-4-(2-fluorophenyl)pyridin-3-yl)carbamoyl)pyrimidin-2-yl)-L-valinate

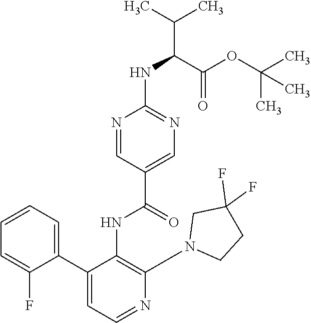 [SMILES: CC(C)[C@H](Nc1ncc(C(=O)Nc2c(-c3ccccc3F)ccnc2N2CCC(F)(F)C2)cn1)C(=O)OC(C)(C)C]

To a mixture of 2-chloro-N-[2-(3,3-difluoropyrrolidin-1-yl)-4-(2-fluorophenyl)-3-pyridyl]pyrimidine-5-carboxamide (0.10 g, 0.18 mmol), L-valine tert-butyl ester hydrochloride (0.074 g, 0.35 mmol), and anhydrous N,N-dimethylformamide (3.1 mL) was added anhydrous potassium carbonate (0.073 g, 0.53 mmol). The reaction vessel was sealed and stirred at 50° C. overnight. A further aliquot of L-valine tert-butyl ester hydrochloride (0.074 g, 0.35 mmol) and anhydrous potassium carbonate (0.073 g, 0.53 mmol) were added to the reaction mixture. The mixture was resealed and heated to 50° C. for a further 3 days. The reaction mixture was cooled to ambient temperature and diluted with ethyl acetate (120 mL). The organic layer was washed with saturated ammonium chloride (2×50 mL), dried over anhydrous magnesium sulfate, filtered, and concentrated in vacuo. The residue was purified by column chromatography, eluting with a gradient of 10 to 100% of ethyl acetate in heptane, to provide the title compound as a colorless solid (0.090 g, 90% yield): ¹H-NMR (400 MHz; CDCl₃) δ 8.57-8.52 (m, 2H), 8.27 (d, J=5.0 Hz, 1H), 7.38-7.33 (m, 2H), 7.21 (td, J=7.5, 1.0 Hz, 2H), 7.15-7.10 (m, 1H), 6.78 (d, J=4.9 Hz, 1H), 5.97-5.94 (m, 1H), 3.94-3.87 (m, 1H), 3.87-3.82 (m, 2H), 3.19 (d, J=4.8 Hz, 2H), 2.45-2.34 (m, 2H), 2.06-1.98 (m, 1H), 1.02-1.00 (m, 9H), 0.93-0.90 (m, 6H); MS (ES+) m/z 571.6 (M+1).

### Step 2. Preparation of (5-((2-(3,3-difluoropyrrolidin-1-yl)-4-(2-fluorophenyl)pyridin-3-yl)carbamoyl)pyrimidin-2-yl)-L-valine

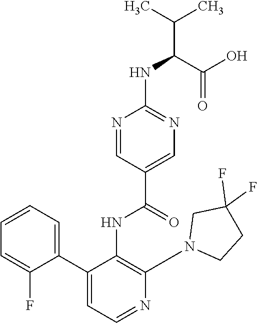 [SMILES: CC(C)[C@H](Nc1ncc(C(=O)Nc2c(-c3ccccc3F)ccnc2N2CCC(F)(F)C2)cn1)C(=O)O]

To a mixture of tert-butyl (5-((2-(3,3-difluoropyrrolidin-1-yl)-4-(2-fluorophenyl)pyridin-3-yl)carbamoyl)pyrimidin-2-yl)-L-valinate (0.090 g, 0.16 mmol), and anhydrous dichloromethane (5.0 mL) was added trifluoroacetic acid (5.0 mL). The reaction vessel was sealed and stirred at ambient temperature overnight. The reaction mixture was concentrated in vacuo. The residue was purified by reverse phase column chromatography, eluting with a gradient of 10 to 95% of acetonitrile in water, to provide the title compound as a colorless solid (0.16 g, 44% yield): ¹H-NMR (400 MHz; CDCl₃) S 12.54-12.49 (m, 1H), 9.71 (s, 1H), 8.54 (s, 2H), 8.18 (d, J=5.0 Hz, 1H), 7.97 (d, J=7.9 Hz, 1H), 7.38-7.33 (m, 1H), 7.30-7.23 (m, 2H), 7.18 (qd, J=7.6, 0.8 Hz, 1H), 6.79 (d, J=4.9 Hz, 1H), 4.25-4.21 (m, 1H), 3.95-3.82 (m, 2H), 3.81-3.69 (m, 2H), 2.50-2.38 (m, 2H), 2.20-2.12 (m, 1H), 1.00-0.93 (m, 6H); MS (ES+) m/z 515.2 (M+1).

### Example 262

### Synthesis of N-[2-(3,3-difluoropyrrolidin-1-yl)-4-(2-fluorophenyl)-3-pyridyl]-5-fluoro-6-(1-hydroxy-1-methyl-ethyl)pyridine-3-carboxamide

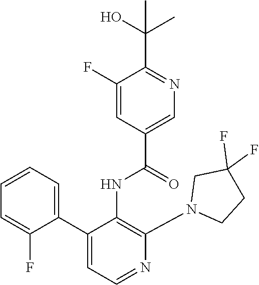 [SMILES: CC(C)(O)c1ncc(C(=O)Nc2c(-c3ccccc3F)ccnc2N2CCC(F)(F)C2)cc1F]

To a mixture of 2-(3,3-difluoropyrrolidin-1-yl)-4-(2-fluorophenyl)pyridin-3-amine hydrochloride (0.10 g, 0.30 mmol), 2-chloro-1-methylpyridinium iodide (0.19 g, 0.76 mmol), 5-fluoro-6-(1-hydroxy-1-methyl-ethyl)pyridine-3-carboxylic acid (0.079 g, 0.39 mmol), and anhydrous tetrahydrofuran (6.1 mL) was added N,N-diisopropylethylamine (0.39 g, 3.0 mmol). The reaction vessel was sealed and heated to 60° C. for 2 h. After cooling to ambient temperature, the reaction mixture was diluted with methanol (5 mL) and 5M sodium hydroxide (1 mL). The reaction mixture was sealed and heated to 50° C. for 30 min. After cooling to ambient temperature, the reaction mixture was diluted with ethyl acetate (100 mL). The organic layer was washed with 1M sodium hydroxide (50 mL), saturated ammonium chloride (50 mL), dried over anhydrous magnesium sulfate, filtered, and concentrated in vacuo. The residue was purified by column chromatography, eluting with a gradient of 10 to 80% of ethyl acetate in heptane, to provide the title compound as a clear, colorless solid (0.12 g, 83% yield): ¹H-NMR (400 MHz; DMSO-d₆) δ 10.14 (s, 1H), 8.57 (t, J=1.6 Hz, 1H), 8.21 (d, J=5.0 Hz, 1H), 7.81 (dd, J=11.9, 1.8 Hz, 1H), 7.40-7.34 (m, 1H), 7.31 (td, J=7.5, 1.4 Hz, 1H), 7.29-7.23 (m, 1H), 7.21-7.17 (m, 1H), 6.82 (d, J=4.9 Hz, 1H), 5.33-5.31 (m, 1H), 3.95-3.82 (m, 2H), 3.82-3.70 (m, 2H), 2.50-2.39 (m, 3H), 1.52-1.46 (m, 6H); MS (ES+) m/z 475.2 (M+1).

### Example 263

### Synthesis of N-[4-(2,5-difluorophenyl)-2-(4,4-difluoro-1-piperidyl)-3-pyridyl]-2-isopropyl-pyrimidine-5-carboxamide

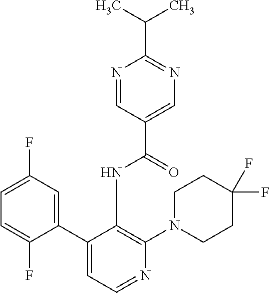 [SMILES: CC(C)c1ncc(C(=O)Nc2c(-c3cc(F)ccc3F)ccnc2N2CCC(F)(F)CC2)cn1]

### Step 1. Preparation of 4-(2,5-difluorophenyl)-2-(4,4-difluoropiperidin-1-yl)-3-nitropyridine

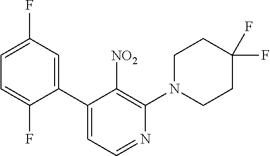 [SMILES: O=[N+]([O-])c1c(-c2cc(F)ccc2F)ccnc1N1CCC(F)(F)CC1]

To a solution of 2-chloro-4-(2,5-difluorophenyl)-3-nitropyridine (0.15 g, 0.55 mmol) in N,N-dimethylformamide (3.0 mL) were added 4-fluoropiperidine hydrochloride (0.18 g, 1.1 mmol) and potassium carbonate (0.23 g, 1.7 mmol). The mixture was stirred at 70° C. for 16 h. The reaction mixture was diluted with water (5 mL) and extracted with ethyl acetate (3×10 mL). The combined organic layers were washed with brine (2×10 mL), dried over sodium sulfate, filtered, and concentrated in vacuo. The residue was purified by column chromatography, eluting with a gradient of 0 to 30% ethyl acetate in petroleum ether to provide the title compound as a brown solid (0.120 g, 57% yield): ¹H-NMR (400 MHz; CDCl₃) δ 8.36 (d, J=4.8 Hz, 1H), 7.27-7.10 (m, 2H), 6.99-6.94 (m, 1H), 6.80 (d, J=5.2 Hz, 1H), 3.58-3.48 (m, 4H), 2.15-2.07 (m, 4H).

### Step 2. Preparation of 4-(2,5-difluorophenyl)-2-(4,4-difluoropiperidin-1-yl)pyridin-3-amine

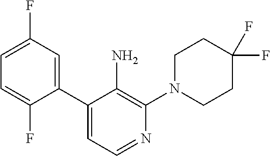 [SMILES: Nc1c(-c2cc(F)ccc2F)ccnc1N1CCC(F)(F)CC1]

To a solution of 4-(2,5-difluorophenyl)-2-(4,4-difluoropiperidin-1-yl)-3-nitropyridine (0.12 g, 0.34 mmol) in ethanol (5 mL) and water (3 mL) was added ammonium chloride (0.090 g, 1.7 mmol) and ferrous powder (0.19 g, 3.4 mmol). The mixture was stirred at 70° C. for 1 h. The reaction mixture was filtrated through diatomaceous earth (i.e., Celite®). The filter cake was washed with dichloromethane (2×15 mL). The filtrate was concentrated in vacuo. The residue was purified by column chromatography, eluting with a gradient of 0 to 30% ethyl acetate in petroleum ether to furnish the title compound as a brown solid (0.056 g, 46% yield): ¹H-NMR (400 MHz; DMSO-d₆) δ 7.62 (d, J=5.2 Hz, 1H), 7.41-7.35 (m, 1H), 7.34-7.30 (m, 1H), 7.29-7.24 (m, 1H), 6.79 (d, J=4.8 Hz, 1H), 4.69 (s, 2H), 3.12 (t, J=5.2 Hz, 4H), 2.25-2.12 (m, 4H).

### Step 3. Preparation of N-[4-(2,5-difluorophenyl)-2-(4,4-difluoro-1-piperidyl)-3-pyridyl]-2-isopropyl-pyrimidine-5-carboxamide

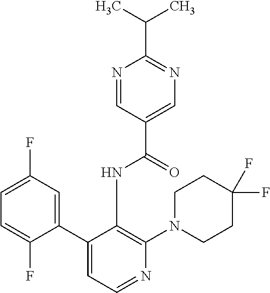 [SMILES: CC(C)c1ncc(C(=O)Nc2c(-c3cc(F)ccc3F)ccnc2N2CCC(F)(F)CC2)cn1]

To a solution of 2-isopropylpyrimidine-5-carboxylic acid (0.038 g, 0.23 mmol) and 2-chloro-1-methyl-pyridin-1-ium iodide (0.059 g, 0.23 mmol) in tetrahydrofuran (3 mL) was added N,N-diisopropylethylamine (0.079 g, 0.62 mmol). After stirring at 20° C. for 30 min, 4-(2,5-difluorophenyl)-2-(4,4-difluoropiperidin-1-yl)pyridin-3-amine (0.050 g, 0.15 mmol) was added. The resulting mixture was stirred at 70° C. for 16 h. The reaction mixture was diluted with water (5 mL) and extracted with ethyl acetate (3×5 mL). The combined organic layers were washed with brine (2×10 mL), dried over sodium sulfate, filtered, and concentrated in vacuo. The residue was purified by preparative reverse phase HPLC, eluting with a gradient of 46-76% of acetonitrile in water, containing formic acid to afford the title compound as a yellow solid (0.027 g, 37% yield) as a yellow solid: ¹H-NMR (400 MHz; CDCl₃) 1 8.92 (s, 2H), 8.34 (d, J=5.2 Hz, 1H), 7.55 (s, 1H), 7.11-7.00 (m, 4H), 3.36-3.33 (m, 4H), 3.31-3.26 (m, 1H), 2.13-2.03 (m, 4H), 1.37 (d, J=7.2 Hz, 6H); MS (ES+) m/z 474.2 (M+1).

### Example 264

### Synthesis of N-(2-(3,3-difluoropyrrolidin-1-yl)-4-(oxazol-5-yl)pyridin-3-yl)-2-isopropylpyrimidine-5-carboxamide

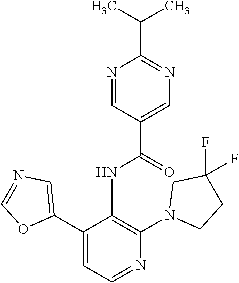 [SMILES: CC(C)c1ncc(C(=O)Nc2c(-c3cnco3)ccnc2N2CCC(F)(F)C2)cn1]

### Step 1. Preparation of potassium 2-(3,3-difluoropyrrolidin-1-yl)-4-iodo-pyridine-3-carboxylate

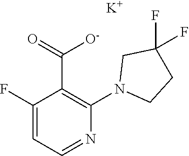 [SMILES: [K+] ; O=C([O-])c1c(F)ccnc1N1CCC(F)(F)C1]

To a mixture of 2-fluoro-4-iodo-pyridine-3-carboxylic acid (12.5 g, 46.8 mmol) and potassium carbonate (12.9 g, 93.6 mmol) in N,N-dimethylformamide (500 mL) was added 3,3-difluoropyrrolidin-1-ium chloride (6.72 g, 46.8 mmol), and the mixture was stirred at 85° C. for 20 h. After cooling to ambient temperature, the mixture was diluted with ethyl acetate (2500 mL) and filtered through diatomaceous earth (i.e., Celite®) washing with ethyl acetate (500 mL), and the filtrate was concentrated in vacuo. The residue was stirred in a mixture of ethyl acetate (20 mL) and diethyl ether (250 mL) for 30 minutes and the solid was filtered, washing with diethyl ether (50 mL). The residue was dried in vacuo, to afford the title compound as a brown solid (9.57 g, 47% yield): ¹H NMR (400 MHz, DMSO-d₆) δ 7.45 (d, J=5.2 Hz, 1H), 6.94 (d, J=5.2 Hz, 1H), 3.95 (t, J=13.9 Hz, 2H), 3.73 (t, J=7.3 Hz, 2H), 2.38 (tt, J=14.4, 7.3 Hz, 2H); MS (ES+) m/z 355.2 (M+1).

### Step 2. Preparation of [2-(3,3-difluoropyrrolidin-1-yl)-4-iodo-3-pyridyl]ammonium chloride

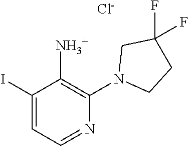 [SMILES: [NH3+]c1c(I)ccnc1N1CCC(F)(F)C1 ; [Cl-]]

To a mixture of potassium 2-(3,3-difluoropyrrolidin-1-yl)-4-iodo-pyridine-3-carboxylate (7.50 g, 19.1 mmol) and triethylamine (6.66 mL, 47.8 mmol) in N-methylpyrrolidone (190 mL) was added diphenylphosphoryl azide (6.17 mL, 28.7 mmol), and the mixture was stirred at 95° C. for 3 h. After cooling to ambient temperature, the mixture was diluted with saturated aqueous sodium bicarbonate (1000 mL), and the aqueous phase was extracted with ethyl acetate (3×1000 mL). The organic phase was washed with brine (1000 mL), dried over anhydrous sodium sulfate, filtered, and concentrated in vacuo. The residue was purified by column chromatography, eluting with a gradient of 0-40% of ethyl acetate in hexanes. The residue was diluted with diethyl ether (50 mL), and hydrochloric acid (2 M solution in diethyl ether, 11.5 mL, 22.9 mmol) was added. Filtration, washing with diethyl ether (5×100 mL), and drying of the residue in vacuo afforded the title compound as a pink solid (4.80 g, 66%): ¹H NMR (400 MHz, DMSO-d₆) δ 7.44-7.33 (m, 1H), 7.25 (d, J=5.6 Hz, 1H), 6.09 (s, 3H), 3.85 (t, J=13.5 Hz, 2H), 3.59 (dd, J=14.5, 7.4 Hz, 2H), 2.59-2.41 (m, 2H); MS (ES+) m/z 326.3 (M+1).

### Step 3. Preparation of N-[2-(3,3-difluoropyrrolidin-1-yl)-4-iodo-3-pyridyl]-2-isopropyl-pyrimidine 5-carboxamide

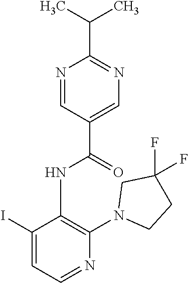 [SMILES: CC(C)c1ncc(C(=O)Nc2c(I)ccnc2N2CCC(F)(F)C2)cn1]

To a solution of 2-(3,3-difluoropyrrolidin-1-yl)-4-iodo-pyridin-3-amine hydrochloride (2.00 g, 5.53 mmol), 2-isopropylpyrimidine-5-carboxylic acid (1.37 g, 8.30 mmol), and 2-chloro-1-methyl-pyridin-1-ium iodide (5.65 g, 22.1 mmol) in tetrahydrofuran (50.0 mL) was added N,N-diisopropylethylamine (3.79 mL, 22.1 mmol), and the mixture was stirred at 65° C. for 20 h. After cooling to ambient temperature, the mixture was diluted with ethyl acetate (50 mL), and the organic phase was washed with saturated aqueous sodium bicarbonate (50 mL). The organic phase was dried over anhydrous sodium sulfate, filtered, and concentrated in vacuo. The residue was diluted with methanol (100 mL), filtered, washing with methanol (50 mL), and the solid was concentrated in vacuo to afford the title compound as a colorless solid (1.73 g, 66% yield): ¹H NMR (400 MHz, CDCl₃) 59.20 (s, 2H), 7.78 (d, J=4.8 Hz, 1H), 7.37 (s, 1H), 7.28 (d, J=5.1 Hz, 1H), 3.92-3.73 (m, 4H), 3.37-3.26 (m, 1H), 2.42-2.30 (m, 2H), 1.41 (d, J=6.9 Hz, 6H); MS (ES+) m/z 474.4 (M+1).

### Step 4. Preparation of N-(2-(3,3-difluoropyrrolidin-1-yl)-4-(oxazol-5-yl)pyridin-3-yl)-2-isopropylpyrimidine-5-carboxamide

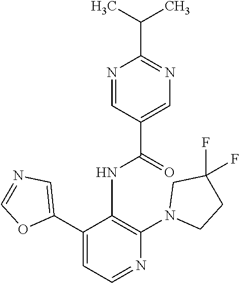 [SMILES: CC(C)c1ncc(C(=O)Nc2c(-c3cnco3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.080 g, 0.169 mmol), 5-(4,4,5,5-tetramethyl-[1,3,2]dioxaborolan-2-yl)-oxazole (0.049 g, 0.254 mmol), and potassium carbonate (0.070 g, 0.507 mmol) in degassed 1,4-dioxane (1.00 mL) and water (0.110 mL) was added [1,1′ bis(diphenylphosphino)ferrocene]dichloropalladium(II), complex with dichloromethane (0.014 g, 0.017 mmol) and the mixture was stirred at 100° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. Purification of the residue by preparative reverse phase HPLC, eluting with a gradient of 10-95% acetonitrile in water 0.5% formic acid, to provide the title compound as a colorless solid (0.0202 g, 28% yield): ¹H-NMR (300 MHz; DMSO-d₆): 5 10.53-10.49 (S, H), 9.27 (s, 2H), 8.55 (s, 1H), 8.24 (d, J=5.2 Hz, 1H), 7.57 (s, 1H), 7.17 (d, J=5.2 Hz, 1H), 3.93-3.71 (m, 4H), 3.25 (dt, J=13.9, 7.0 Hz, 1H), 2.46-2.37 (m, 2H), 1.34 (d, J=6.9 Hz, 6H); MS (ESI+) m/z 415.2 (M+1).

### Examples 265-278

In a similar manner as described in EXAMPLE 191, utilizing the appropriately substituted starting materials and intermediates, the following compounds were

| Example | Structure | Amount | MS (ES+) | |
|---|---|---|---|---|
| No. | Name | Yield | m/z | ¹H NMR |
| 265 | 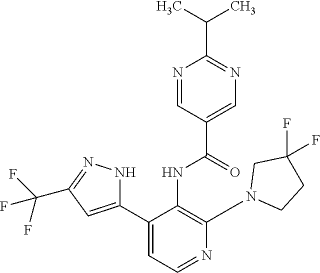 [SMILES: CC(C)c1ncc(C(=O)Nc2c(-c3cc(C(F)(F)F)n[nH]3)ccnc2N2CCC(F)(F)C2)cn1] N-(2-(3,3-difluoropyrrolidin-1-yl)-4- (3-(trifluoromethyl)-1H-pyrazol-5-yl)pyridin-3-yl)- 2-isopropylpyrimidine-5-carboxamide | 0.011 g 6.6% | 482.2 (M + 1) | (300 MHz, DMSO-d₆) δ 10.49 (s, 1H), 9.14 (s, 2H), 8.26 (d, J = 5.0 Hz, 1H), 7.04 (d, J = 5.1 Hz, 1H), 6.89 (s, 1H), 3.95-3.78 (m, 4H), 3.20 (m, 1H), 2.47-2.37 (m, 2H), 1.32 (s, 3H), 1.29 (s, 3H) |
| 266 | 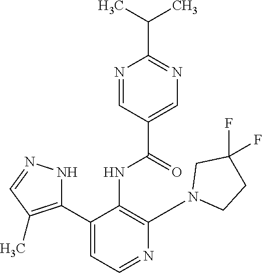 [SMILES: Cc1cn[nH]c1-c1ccnc(N2CCC(F)(F)C2)c1NC(=O)c1cnc(C(C)C)nc1] N-[2-(3,3-difluoropyrrolidin-1-yl)-4- (4-methyl-1H-pyrazol-5-yl)-3-pyridyl]-2-isopropyl- pyrimidine-5-carboxamide | 0.016 g 22% | 428.2 (M + 1) | (300 MHz, DMSO-d₆) δ 10.28 (s, 1H), 9.02 (s, 2H), 8.17 (d, J = 5.0 Hz, 1H), 7.48 (s, 1H), 6.84 (d, J = 5.0 Hz, 1H), 3.92-3.71 (m, 4H), 3.19 (dt, J = 13.8, 6.9 Hz, 1H), 2.47-2.35 (m, 2H), 1.98 (s, 3H), 1.30 (s, 3H), 1.28 (s, 3H) |
| 267 | 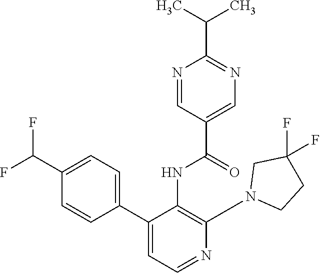 [SMILES: CC(C)c1ncc(C(=O)Nc2c(-c3ccc(C(F)F)cc3)ccnc2N2CCC(F)(F)C2)cn1] N-[4-[4-(difluoromethyl)phenyl]- 2-(3,3-difluoropyrrolidin-1-yl)-3-pyridyl]-2- isopropyl-pyrimidine-5-carboxamide | 0.066 g 83% | 474.3 (M + 1) | (300 MHz, DMSO-d₆) δ 10.23 (s, 1H), 8.95 (s, 2H), 8.22 (d, J = 5.0 Hz, 1H), 7.59 (d, J = 8.3 Hz, 2H), 7.53 (d, J = 8.4 Hz, 2H), 6.83 (d, J = 5.0 Hz, 1H), 3.93- 3.72 (m, 4H), 3.18 (7, J = 6.9 Hz, 1H), 2.42 (dt, J = 14.2, 7.2 Hz, 2H), 1.28 (s, 3H), 1.26 (s, 3H) |
| 268 | 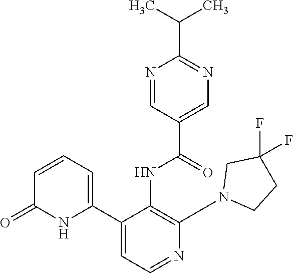 [SMILES: CC(C)c1ncc(C(=O)Nc2c(-c3cccc(=O)[nH]3)ccnc2N2CCC(F)(F)C2)cn1] N-[2-(3,3-difluoropyrrolidin-1-yl)-4- (6-oxo-1H-pyridin-2-yl)-3-pyridyl]-2-isopropyl- pyrimidine-5-carboxamide | 0.013 g 17% | 441.2 (M + 1) | (300 MHz, DMSO-d₆) δ 11.63 (s, 1H), 10.21 (s, 1H), 9.04 (s, 2H), 8.22 (d, J = 5.0 Hz, 1H), 7.47-7.41 (m, 1H), 6.88 (d, J = 5.0 Hz, 1H), 6.34-6.30 (m, 1H), 3.93-3.83 (m, 2H), 3.76-3.71 (m, 2H), 3.20 (dt, J = 13.8, 6.9 Hz, 1H), 2.41 (dt, J = 14.3, 7.2 Hz, 2H), 1.29 (d, J = 6.9 Hz, 6H) |
| 269 | 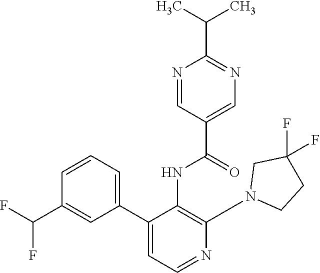 [SMILES: CC(C)c1ncc(C(=O)Nc2c(-c3cccc(C(F)F)c3)ccnc2N2CCC(F)(F)C2)cn1] N-[4-[3-(difluoromethyl)phenyl]- 2-(3,3-difluoropyrrolidin-1-yl)-3-pyridyl]-2- isopropyl-pyrimidine-5-carboxamide | 0.061 g 76% | 474.2 (M + 1) | (300 MHz, DMSO-d₆) δ 10.22 (s, 1H), 8.93 (s, 2H), 8.23 (d, J = 5.0 Hz, 1H), 7.61 (d, J = 1.0 Hz, 1H), 7.56-7.52 (m, 3H), 7.07 (t, J = 55.7 Hz, 1H), 6.84 (d, J = 5.0 Hz, 1H), 3.97-3.69 (m, 4H), 3.17 (7, J = 6.9 Hz, 1H), 2.42 (dt, J = 14.3, 7.1 Hz, 2H), 1.26 (d, J = 6.9 Hz, 6H) |
| 270 | 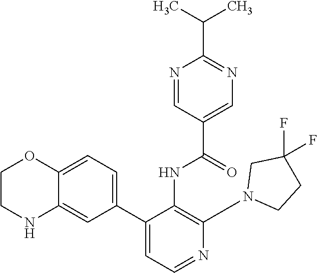 [SMILES: CC(C)c1ncc(C(=O)Nc2c(-c3ccc4c(c3)NCCO4)ccnc2N2CCC(F)(F)C2)cn1] N-[2-(3,3-difluoropyrrolidin-1-yl)-4- (3,4-dihydro-2H-1,4-benzoxazin-6-yl)-3- pyridyl]-2-isopropyl-pyrimidine-5-carboxamide | 0.061 g 74% | 481.2 (M + 1) | (300 MHz, DMSO-d₆) δ 10.09 (s, 1H, -NH), 8.99 (s, 2H), 8.12 (d, J = 5.0 Hz, 1H), 6.71 (d, J = 5.0 Hz, 1H), 6.57 (m, 3H), 5.87 (s, 1H -NH), 4.08 (t, J = 4.1 Hz, 2H), 3.95-3.65 (m, 4H), 3.24-3.15 (m, 3H), 2.46-2.35 (m, 2H), 1.29 (d, J = 6.9 Hz, 6H) |
| 271 | 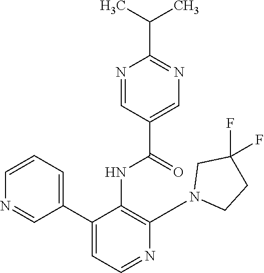 [SMILES: CC(C)c1ncc(C(=O)Nc2c(-c3cccnc3)ccnc2N2CCC(F)(F)C2)cn1] N-[2-(3,3-difluoropyrrolidin-1-yl)-4- (3-pyridyl)-3-pyridyl]-2-isopropyl-pyrimidine-5- carboxamide | 0.031 g 49% | 425.2 (M + 1) | (400 MHz, DMSO-d₆) δ 10.29 (s, 1H), 8.95 (s, 2H), 8.58 (d, J = 2.1 Hz, 1H), 8.52 (dd, J = 4.8, 1.4 Hz, 1H), 8.25 (d, J = 4.9 Hz, 1H), 7.80 (dt, J = 7.9, 1.9 Hz, 1H), 7.42 (dd, J = 7.9, 4.8 Hz, 1H), 6.87 (d, J = 5.0 Hz, 1H), 3.96-3.72 (m, 4H), 3.17 (dq, J = 13.8, 6.9 Hz, 1H), 2.48-2.39 (m, 2H), 1.28 (d, J = 6.9 Hz, 6H) |
| 272 | 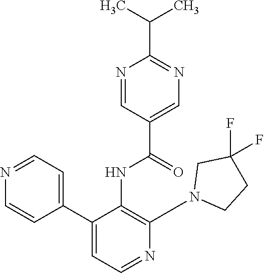 [SMILES: CC(C)c1ncc(C(=O)Nc2c(-c3ccncc3)ccnc2N2CCC(F)(F)C2)cn1] N-[2-(3,3-difluoropyrrolidin-1-yl)-4- (4-pyridyl)-3-pyridyl]-2-isopropyl-pyrimidine-5- carboxamide | 0.043 g 68% | 425.2 (M + 1) | (400 MHz, DMSO-d₆) δ 10.27 (s, 1H), 8.96 (s, 2H), 8.58 (dd, J = 4.4, 1.6 Hz, 2H), 8.25 (d, J = 5.0 Hz, 1H), 7.39- 7.37 (m, 2H), 6.84 (d, J = 5.0 Hz, 1H), 3.96- 3.73 (m, 4H), 3.19 (7, J = 6.9 Hz, 1H), 2.44 (td, J = 14.2, 7.2 Hz, 2H), 1.28 (d, J = 6.9 Hz, 6H) |
| 273 | 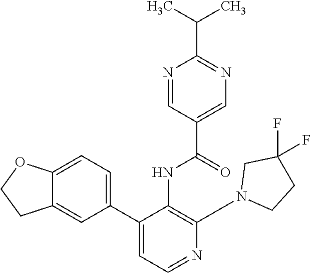 [SMILES: CC(C)c1ncc(C(=O)Nc2c(-c3ccc4c(c3)CCO4)ccnc2N2CCC(F)(F)C2)cn1] N-(2-(3,3-difluoropyrrolidin-1-yl)-4- (2,3-dihydrobenzofuran-5-yl)pyridin-3-yl)-2- isopropylpyrimidine-5-carboxamide | 0.062 g 90% | 466.2 (M + 1) | (400 MHz, DMSO-d₆) δ 10.12 (s, 1H), 8.99 (s, 2H), 8.15 (d, J = 5.0 Hz, 1H), 7.26 (s, 1H), 7.16 (dd, J = 8.2, 1.7 Hz, 1H), 6.78 (d, J = 4.8 Hz, 1H), 4.50 (t, J = 8.7 Hz, 2H), 3.94-3.67 (m, 4H), 3.21 (dt, J = 13.8, 6.9 Hz, 1H), 3.16- 3.10 (m, 2H), 2.47-2.38 (m, 2H), 1.29 (d, J = 6.9 Hz, 6H) |
| 274 | 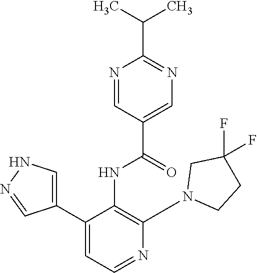 [SMILES: CC(C)c1ncc(C(=O)Nc2c(-c3cn[nH]c3)ccnc2N2CCC(F)(F)C2)cn1] N-(2-(3,3-difluoropyrrolidin-1-yl)-4- (1H-pyrazol-4-yl)pyridin-3-yl)-2- isopropylpyrimidine-5-carboxamide | 0.019 g 33% | 414.2 (M + 1) | (400 MHz, DMSO-d₆) δ 13.05 (s, 1H), 10.38 (s, 1H), 9.24 (s, 2H), 8.10 (d, J = 5.1 Hz, 1H), 7.93 (s, 2H), 7.03 (d, J = 5.2 Hz, 1H), 3.94- 3.77 (m, 3H), 3.70-3.66 (m, 1H), 3.24 (dquintet, J = 13.8, 6.9 Hz, 1H), 2.47-2.37 (m, 2H), 1.33 (d, J = 6.9 Hz, 6H) |
| 275 | 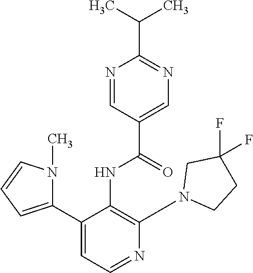 [SMILES: CC(C)c1ncc(C(=O)Nc2c(-c3cccn3C)ccnc2N2CCC(F)(F)C2)cn1] N-(2-(3,3-difluoropyrrolidin-1-yl)-4- (1-methyl-1H-pyrrol-2-yl)pyridin-3-yl)-2- isopropylpyrimidine-5-carboxamide | 0.038 g 70% | 427.2 (M + 1) | (400 MHz, DMSO-d₆) δ 10.05 (s, 1H), 8.98 (s, 2H), 8.17 (d, J = 5.0 Hz, 1H), 6.82 (d, J = 5.0 Hz, 1H), 6.80 (dd, J = 2.2, 2.1 Hz, 1H), 6.11 (dd, J = 3.6, 1.7 Hz, 1H), 5.97 (dd, J = 3.5, 2.7 Hz, 1H), 3.91-3.72 (m, 4H), 3.49 (s, 3H), 3.20 (dquintet, J = 13.8, 6.9 Hz, 1H), 2.43 (td, J = 14.2, 7.2 Hz, 2H), 1.29 (d, J = 6.9 Hz, 6H) |
| 276 | 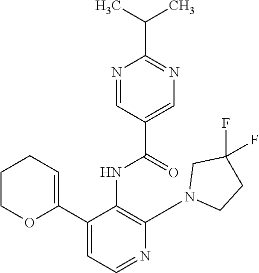 [SMILES: CC(C)c1ncc(C(=O)Nc2c(C3=CCCCO3)ccnc2N2CCC(F)(F)C2)cn1] N-(2-(3,3-difluoropyrrolidin-1-yl)-4- (3,4-dihydro-2H-pyran-6-yl)pyridin-3-yl)-2- isopropylpyrimidine-5-carboxamide | 0.014 g 22% | 430.2 (M + 1) | (400 MHz, DMSO-d₆) δ 10.07 (s, 1H), 9.20 (s, 2H), 8.09 (d, J = 5.0 Hz, 1H), 6.78 (d, J = 5.0 Hz, 1H), 5.07 (t, J = 3.8 Hz, 1H), 3.92-3.81 (m, 4H), 3.76-3.67 (m, 2H), 3.22 (dt, J = 13.7, 6.8 Hz, 1H), 2.41 (tt, J = 14.2, 7.1 Hz, 2H), 2.02 (q, J = 5.1 Hz, 2H), 1.74-1.68 (m, 2H), 1.32 (d, J = 6.9 Hz, 6H) |
| 277 | 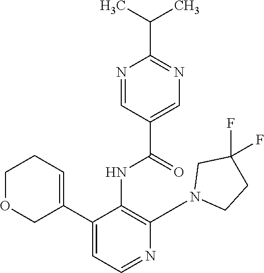 [SMILES: CC(C)c1ncc(C(=O)Nc2c(C3=CCCOC3)ccnc2N2CCC(F)(F)C2)cn1] N-(2-(3,3-difluoropyrrolidin-1-yl)-4- (3,6-dihydro-2H-pyran-5-yl)pyridin-3-yl)-2- isopropylpyrimidine-5-carboxamide | 0.11 g 60% | 430.2 (M + 1) | (400 MHz, DMSO-d₆) δ 10.10 (s, 1H), 9.17 (s, 2H), 8.10 (d, J = 5.0 Hz, 1H), 6.70 (d, J = 5.0 Hz, 1H), 5.81 (dt, J = 3.6, 1.9 Hz, 1H), 4.13-4.08 (m, 2H), 3.89-3.82 (m, 2H), 3.72 (s, 2H), 3.61 (t, J = 5.4 Hz, 2H), 3.26 (dt, J = 13.8, 6.9 Hz, 1H), 2.42 (tt, J = 14.2, 7.1 Hz, 2H), 2.03-1.99 (m, 2H), 1.32 (d, J = 6.9 Hz, 6H) |
| 278 | 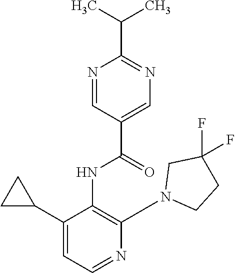 [SMILES: CC(C)c1ncc(C(=O)Nc2c(C3CC3)ccnc2N2CCC(F)(F)C2)cn1] N-[4-cyclopropyl-2-(3,3-difluoropyrrolidin-1-yl)-3- pyridyl]-2-isopropyl-pyrimidine-5- carboxamide | 0.0072 g 12% | 388.2 (M + 1) | (400 MHz, DMSO-d₆) δ 10.27 (s, 1H), 9.28 (s, 2H), 7.99 (d, J = 5.1 Hz, 1H), 6.36 (d, J = 5.2 Hz, 1H), 3.88-3.79 (m, 2H), 3.73-3.67 (m, 2H), 3.24 (dt, J = 13.7, 6.8 Hz, 1H), 2.40 (tt, J = 14.0, 7.0 Hz, 2H), 1.95-1.88 (m, 1H), 1.32 (d, J = 6.9 Hz, 6H), 0.93-0.91 (m, 2H), 0.69-0.68 (m, 2H) |

### Example 279

### Synthesis of N-[2-(3,3-difluoropyrrolidin-1-yl)-4-(1H-imidazol-4-yl)-3-pyridyl]-2-isopropYpy-yrimidine-5-carboxamide

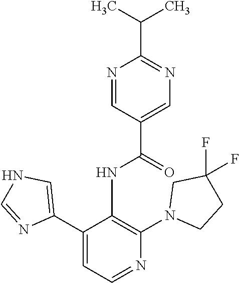 [SMILES: CC(C)c1ncc(C(=O)Nc2c(-c3c[nH]cn3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.080 g, 0.17 mmol), 1-tetrahydropyran-2-yl-4-(4,4,5,5-tetramethyl-1,3,2-dioxaborolan-2-yl)imidazole (0.071 g, 0.25 mmol), and potassium carbonate (0.070 g, 0.51 mmol) in degassed 1,4-dioxane (1.0 mL) and water (0.11 mL) was added [1,1′-bis(diphenylphosphino)ferrocene]dichloropalladium(II), complex with dichloromethane (0.014 g, 0.017 mmol) and the mixture was stirred at 100° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. The crude residue was re-dissolved in methanol and a few drops of concentrated HCl was added. The solution was heated to 50 degrees for 1 h and concentrated in vacuo. Purification of the residue by preparative reverse phase HPLC, eluting with a gradient of 10-95% acetonitrile in water 0.5% formic acid, provided the title compound as a colorless solid (0.027 g, 39% yield): ¹H-NMR (300 MHz; DMSO-d₆) δ 12.27 (s, 1H), 10.48 (s, 1H), 9.28 (s, 2H), 8.09 (d, J=5.2 Hz, 1H), 7.80-7.78 (m, 1H), 7.53 (s, 1H), 7.32 (d, J=5.3 Hz, 1H), 3.89-3.79 (m, 2H), 3.74-3.67 (m, 2H), 3.29-3.20 (m, 1H), 2.45-2.34 (m, 2H), 1.33 (d, J=6.9 Hz, 6H); MS (ESI+) m/z 414.2 (M+1).

### Example 280

### Synthesis of N-[2-(3,3-difluoropyrrolidin-1-yl)-4-oxazol-2-yl-3-pyridyl]-2-isopropyl-pyrimidine-5-carboxamide

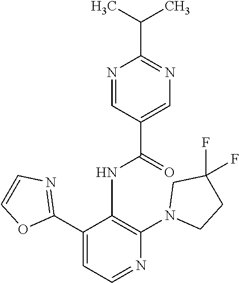 [SMILES: CC(C)c1ncc(C(=O)Nc2c(-c3ncco3)ccnc2N2CCC(F)(F)C2)cn1]

To a solution of oxazole (0.012 mL, 0.18 mmol) in THF (1.0 mL) was added n-butyllithium (1.6M in hexane) (0.13 mL, 0.20 mmol) at −78° C., followed by zinc chloride (1 M in diethyl ether) (0.34 mL, 0.34 mmol). Stirring was continued at the ambient temperature for 30 min. To the mixture was added N-[2-(3,3-difluoropyrrolidin-1-yl)-4-iodo-3-pyridyl]-2-isopropyl-pyrimidine-5-carboxamide (0.080 g, 0.17 mmol) and tetrakis(triphenylphosphine)palladium (0) (0.020 g, 0.017 mmol) and the mixture was stirred at 60° C. for 5 h. The mixture was cooled to ambient temperature and partitioned between water and ethyl acetate. The organic layer was dried over anhydrous magnesium sulfate and concentrated in vacuo. Purification of the residue by preparative reverse phase HPLC, eluting with a gradient of 10-95% acetonitrile in water containing 0.5% formic acid, provided the title compound as a colorless solid (0.017 g, 24% yield): ¹H-NMR (300 MHz; DMSO-d₆) δ 10.43 (s, 1H), 9.23 (s, 2H), 8.29-8.28 (m, 2H), 7.43 (d, J=0.5 Hz, 1H), 7.36 (d, J=5.1 Hz, 1H), 3.97-3.87 (m, 2H), 3.81-3.76 (m, 2H), 3.29-3.20 (m, 1H), 2.47-2.37 (m, 2H), 1.33 (d, J=6.9 Hz, 6H); MS (ESI+) m/z 415.2 (M+1).

### Example 281

### Synthesis of N-[2-(3,3-difluoropyrrolidin-1-yl)-4-[4-(trifluoromethyl)-1H-pyrazol-5-yl]-3-pyridyl]-2-isopropyl-pyrimidine-5-carboxamide

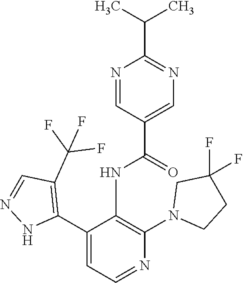 [SMILES: CC(C)c1ncc(C(=O)Nc2c(-c3[nH]ncc3C(F)(F)F)ccnc2N2CCC(F)(F)C2)cn1]

### Step 1. Preparation of N-[2-(3,3-difluoropyrrolidin-1-yl)-4-[4-(trifluoromethyl)-2-(2-trimethylsilylethoxymethyl)pyrazol-3-yl]-3-pyridyl]-2-isopropyl-pyrimidine-5-carboxamide

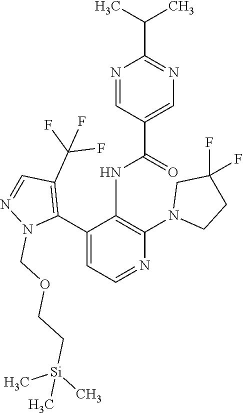 [SMILES: CC(C)c1ncc(C(=O)Nc2c(-c3c(C(F)(F)F)cnn3COCC[Si](C)(C)C)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.135 g, 0.29 mmol), trimethyl-[2-[[5-(4,4,5,5-tetramethyl-1,3,2-dioxaborolan-2-yl)-4-(trifluoromethyl)pyrazol-1-yl]methoxy]ethyl]silane (0.168 g, 0.43 mmol), and potassium carbonate (0.118 g, 0.86 mmol) in degassed 1,4-dioxane (1.7 mL) and water (0.19 mL) was added [1,1′ bis(diphenylphosphino)ferrocene]dichloropalladium(II), complex with dichloromethane (0.023 g, 0.029 mmol) and the mixture was stirred at 100° C. for 16 h. After cooling to ambient temperature, the mixture was diluted with ethyl acetate (10 mL). The mixture was passed through a bed of diatomaceous earth (i.e., Celite®). The solid was washed with ethyl acetate (20 mL) and the filtrate was concentrated in vacuo. The residue was used as is in the next step (0.126 g, 72%): MS (ESI+) m/z 612.4 (M+1).

### Step 2. Preparation of N-[2-(3,3-difluoropyrrolidin-1-yl)-4-[4-(trifluoromethyl)-1H-pyrazol-5-yl]-3-pyridyl]-2-isopropyl-pyrimidine-5-carboxamide

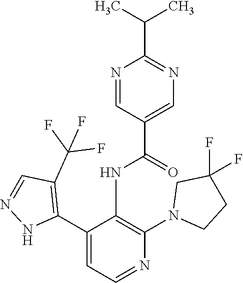 [SMILES: CC(C)c1ncc(C(=O)Nc2c(-c3[nH]ncc3C(F)(F)F)ccnc2N2CCC(F)(F)C2)cn1]

A solution of N-[2-(3,3-difluoropyrrolidin-1-yl)-4-[4-(trifluoromethyl)-2-(2-trimethylsilylethoxymethyl)pyrazol-3-yl]-3-pyridyl]-2-isopropyl-pyrimidine-5-carboxamide (0.126 g, 0.21 mmol) in dichloromethane (1.4 mL) and trifluoroacetic acid (0.80 mL, 10.5 mmol) was stirred at ambient temperature for 4 h. The reaction mixture was concentrated in vacuo and purified by column chromatography, eluting with a gradient of 20% to 65% of ethyl acetate in heptane, to provide the title compound as a colorless solid (0.021 g, 21% yield). ¹H-NMR (300 MHz; DMSO-d₆) δ 13.71 (s, 1H), 10.10 (s, 1H), 8.98 (d, J=8.6 Hz, 2H), 8.44-8.38 (m, 1H), 8.22 (d, J=5.0 Hz, 1H), 6.82 (d, J=4.9 Hz, 1H), 3.94-3.84 (m, 2H), 3.77-3.72 (m, 2H), 3.19 (7, J=6.9 Hz, 1H), 2.47-2.36 (m, 2H), 1.29 (d, J=6.9 Hz, 6H); MS (ESI+) m/z 482.2 (M+1).

### Example 282

### Synthesis of N-(2-(3,3-difluoropyrrolidin-1-yl)-4-(thiazol-2-yl)pyridin-3-yl)-2-isopropylpyrimidine-5-carboxamide

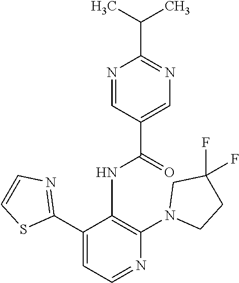 [SMILES: CC(C)c1ncc(C(=O)Nc2c(-c3nccs3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.070 g, 0.15 mmol) in THF (1.2 mL) was added 2-thiazolylzinc bromide in THF (0.5M, 0.35 mL, 0.177 mmol) and stirred for 8 h at 65 degree. Upon completion, the mixture was cooled to ambient temperature and partitioned between water and ethyl acetate. The organic layer was dried over anhydrous magnesium sulfate and concentrated in vacuo. Purification of the residue by preparative reverse phase HPLC, eluting with a gradient of 5-95% acetonitrile in water containing 0.5% formic acid provided the title compound as a colorless solid (0.011 g, 17% yield): ¹H-NMR (400 MHz; DMSO-d₆) δ 10.68 (s, 1H), 9.30 (s, 2H), 8.25 (d, J=5.1 Hz, 1H), 8.01 (d, J=3.2 Hz, 1H), 7.88 (d, J=3.2 Hz, 1H), 7.52 (d, J=5.2 Hz, 1H), 3.96-3.72 (m, 4H), 3.30-3.21 (m, 1H), 2.48-2.41 (m, 2H), 1.34 (d, J=6.9 Hz, 6H); MS (ESI+) m/z 431.2 (M+1).

### Example 283

### Synthesis of N-[2-(3,3-difluoropyrrolidin-1-yl)-4-pyrrolidin-2-yl-3-pyridyl]-2-isopropyl-pyrimidine-5-carboxamide

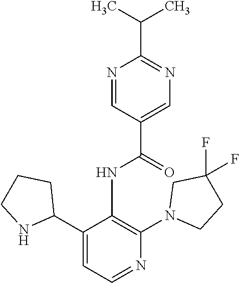 [SMILES: CC(C)c1ncc(C(=O)Nc2c(C3CCCN3)ccnc2N2CCC(F)(F)C2)cn1]

### Step 1. Preparation of tert-butyl 2-(2-(3,3-difluoropyrrolidin-1-yl)-3-(2-isopropylpyrimidine-5-carboxamido)pyridin-4-yl)pyrrolidine-1-carboxylate

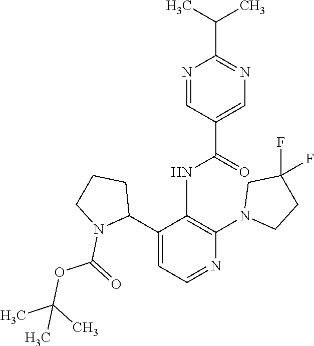 [SMILES: CC(C)c1ncc(C(=O)Nc2c(C3CCCN3C(=O)OC(C)(C)C)ccnc2N2CCC(F)(F)C2)cn1]

To N-[2-(3,3-difluoropyrrolidin-1-yl)-4-iodo-3-pyridyl]-2-isopropyl-pyrimidine-5-carboxamide (0.080 g, 0.17 mmol) was added N-(tert-butoxycarbonyl)-L-proline (0.055 g, 0.25 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0019 g, 0.0017 mmol), dichloro(dimethoxyethane)nickel (0.0037 g, 0.017 mmol), 4-tert-butyl-2-(4-tert-butyl-2-pyridyl)pyridine (0.13 g, 0.50 mmol), cesium carbonate (0.083 g, 0.25 mmol), and N,N-dimethylformamide (4.2 mL). The vial was sealed, and the reaction mixture was stirred in front of Kessil PR160L lights (440 nm) for 24 h. The reaction mixture was diluted with ethyl acetate (15 mL) and washed with saturated ammonium chloride solution (2×20 mL), water (20 mL), and brine (20 mL). The organic layer was dried over anhydrous magnesium sulfate, filtered, and concentrated in vacuo. The crude residue was used as is in the next step (0.079 g, 90%): MS (ESI+) m/z 517.2 (M+1).

### Step 2. Preparation of N-[2-(3,3-difluoropyrrolidin-1-yl)-4-pyrrolidin-2-yl-3-pyridyl]-2-isopropyl-pyrimidine-5-carboxamide

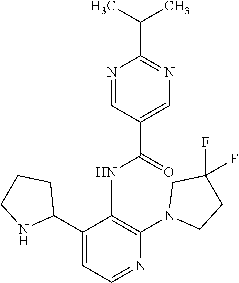 [SMILES: CC(C)c1ncc(C(=O)Nc2c(C3CCCN3)ccnc2N2CCC(F)(F)C2)cn1]

To a solution of tert-butyl 2-(2-(3,3-difluoropyrrolidin-1-yl)-3-(2-isopropylpyrimidine-5-carboxamido)pyridin-4-yl)pyrrolidine-1-carboxylate (0.079 g, 0.15 mmol) in dichloromethane (1.5 mL) and methanol (0.96 mL) was added 4M hydrochloric acid in dioxane (1.5 mL, 0.15 mmol) and stirred at ambient temperature for 1.5 h. The reaction mixture was neutralized with saturated sodium bicarbonate and sodium carbonate solution till the pH reached 6.5. The mixture was concentrated in vacuo and purified by preparative reverse-phase HPLC, using 5% to 60% of acetonitrile in water containing 0.5% formic acid as eluent, afforded the title compound as a colorless solid (0.021 g, 21% yield). ¹H-NMR (400 MHz; DMSO-d₆) δ 9.26 (s, 2H), 8.09 (d, J=5.1 Hz, 1H), 7.05 (d, J=5.1 Hz, 1H), 4.21-4.20 (m, 1H), 3.90-3.64 (m, 5H), 3.24 (tq, J=13.9, 7.0 Hz, 1H), 2.98-2.88 (m, 2H), 2.40 (tt, J=14.0, 7.1 Hz, 2H), 2.09-2.02 (m, 1H), 1.72-1.65 (m, 2H), 1.48-1.39 (m, 1H), 1.33 (d, J=6.9 Hz, 6H); MS (ESI+) m/z 417.2 (M+1).

### Example 284

### Synthesis of N-[2-(3,3-difluoropyrrolidin-1-yl)-4-tetrahydropyran-2-yl-3-pyridyl]-2-isopropyl-pyrimidine-5-carboxamide

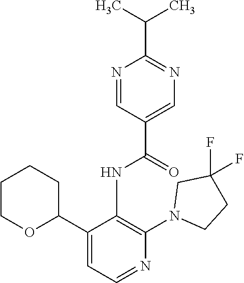 [SMILES: CC(C)c1ncc(C(=O)Nc2c(C3CCCCO3)ccnc2N2CCC(F)(F)C2)cn1]

To N-[2-(3,3-difluoropyrrolidin-1-yl)-4-iodo-3-pyridyl]-2-isopropyl-pyrimidine-5-carboxamide (0.060 g, 0.13 mmol) was added tetrahydropyran-2-carboxylic acid (0.025 g, 0.19 mmol), (4,4″-di-t-butyl-2,2″-bipyridine)bis[3,5-difluoro-2-[5-trifluoromethyl-2-pyridinyl-kN)phenyl-kC]iridium(III) hexafluorophosphate (0.0014 g, 0.0013 mmol), dichloro(dimethoxyethane)nickel (0.0028 g, 0.013 mmol), 4-tert-butyl-2-(4-tert-butyl-2-pyridyl)pyridine (0.051 g, 0.019 mmol), cesium carbonate (0.062 g, 0.19 mmol), and N,N-dimethylformamide (3.2 mL). The vial was sealed, and the reaction mixture was stirred in front of Kessil PR160L lights (440 nm) for 24 h. The reaction mixture was diluted with ethyl acetate (15 mL) and washed with saturated ammonium chloride solution (2×20 mL), water (20 mL), and brine (20 mL). The organic layer was dried over anhydrous magnesium sulfate, filtered, and concentrated in vacuo. Purification of the residue by preparative reverse phase HPLC, eluting with a gradient of 5-95% acetonitrile in water containing 0.5% formic acid, provided the title compound as a yellow solid (0.0080 g, 14%): ¹H-NMR (400 MHz; DMSO-d₆) δ 10.20 (s, 1H), 9.25 (s, 2H), 8.12 (d, J=5.1 Hz, 1H), 6.91 (d, J=5.1 Hz, 1H), 4.36 (m, 1H), 4.00-3.97 (m, 1H), 3.85-3.79 (m, 2H), 3.70-3.66 (m, 2H), 3.48-3.42 (m, 1H), 3.29-3.19 (m, 2H), 2.46-2.35 (m, 2H), 1.79-1.76 (m, 2H), 1.55-1.54 (m, 3H), 1.34 (d, J=6.9 Hz, 6H); MS (ESI+) m/z 432.3 (M+1).

### Examples 285-286

In a similar manner as described in EXAMPLE 191, utilizing the appropriately substituted starting materials and intermediates, the following compounds were

| Example | Structure | | MS (ES+) | |
|---|---|---|---|---|
| No. | Name | Amount | m/z | ¹H NMR |
| 285 | 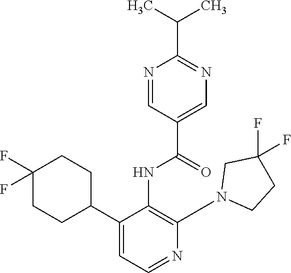 [SMILES: CC(C)c1ncc(C(=O)Nc2c(C3CCC(F)(F)CC3)ccnc2N2CCC(F)(F)C2)cn1] N-[4-(4,4-difluorocyclohexyl)- 2-(3,3-difluoropyrrolidin-1- yl)-3-pyridyl]-2-isopropyl-pyrimidine- 5-carboxamide | 0.0045 g 5.0% | 466.2 (M + 1) | (400 MHz, MeOD) δ 9.27 (s, 2H), 8.46 (s, 1H), 8.12 (d, J = 5.2 Hz, 1H), 6.87 (d, J = 5.2 Hz, 1H), 3.79 (dt, J = 29.8, 10.2 Hz, 4H), 3.38-3.36 (m, 1H), 2.88-2.79 (m, 1H), 2.39 (tt, J = 13.7, 6.9 Hz, 2H), 2.16-2.10 (m, 2H), 1.90-1.73 (m, 6H), 1.41 (d, J = 6.9 Hz, 6H) |
| 286 | 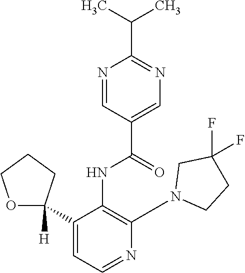 [SMILES: CC(C)c1ncc(C(=O)Nc2c([C@H]3CCCO3)ccnc2N2CCC(F)(F)C2)cn1] N-[2-(3,3-difluoropyrrolidin-1- yl)-4-[(2R)-tetrahydrofuran-2- yl]-3-pyridyl]-2-isopropyl-pyrimidine- 5-carboxamide | 0.0018 g 2.5% | 418.2 (M + 1) | (400 MHz, MeOD) δ 9.32-9.24 (m, 2H), 8.38 (s, 1H), 8.14 (d, J = 5.2 Hz, 1H), 7.00 (d, J = 5.2 Hz, 1H), 5.01 (t, J = 7.3 Hz, 1H), 4.10 (q, J = 7.3 Hz, 1H), 3.92-3.74 (m, 6H), 2.44-2.30 (m, 3H), 2.01-1.94 (m, 2H), 1.78-1.71 (m, 1H), 1.41 (d, J = 6.9 Hz, 6H) |

### Example 287

### Synthesis of N-[2-(3,3-difluoropyrrolidin-1-yl)-4-(2-trimethylsilylethynyl)-3-pyridyl]-2-isopropyl-pyrimidine-5-carboxamide

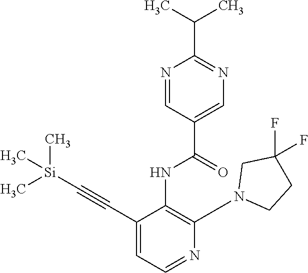 [SMILES: CC(C)c1ncc(C(=O)Nc2c(C#C[Si](C)(C)C)ccnc2N2CCC(F)(F)C2)cn1]

A solution of N-[2-(3,3-difluoropyrrolidin-1-yl)-4-iodo-3-pyridyl]-2-isopropyl-pyrimidine-5-carboxamide (0.12 g, 0.25 mmol), trimethylsilylacetylene (0.070 mL, 0.51 mmol), trans-dichlorobis(triphenylphosphine)palladium (II) (0.0089 g, 0.013 mmol), copper(I) iodide (0.0048 g, 0.025 mmol), and triethylamine (0.18 mL, 1.3 mmol) in THF (1.7 mL) was stirred at 65° C. for 8 h. The volatiles were concentrated in vacuo and the residue was taken up in water and extracted with diethyl ether. The organic layer was dried over anhydrous magnesium sulfate, filtered, and concentrated in vacuo. Purification of the residue by column chromatography (Biotage Sfar KP-Amino D), eluting with a gradient of 0-40% ethyl acetate in heptane provide the title compound as a colorless solid (0.0063 g, 5.4% yield): ¹H-NMR (400 MHz; CDCl₃), 9.27 (s, 2H), 8.09 (m, 1H), 6.87 (d, J=4.4 Hz, 1H), 4.05-3.78 (m, 4H), 3.36 (dquintet, J=13.8, 6.9 Hz, 1H), 2.44-2.34 (m, 2H), 1.41 (d, J=6.9 Hz, 6H), 0.13-0.06 (m, 9H); MS (ESI+) m/z 444.2 (M+1).

### Example 288

### Synthesis of N-[2-(3,3-difluoropyrrolidin-1-yl)-4-(1H-indol-2-yl)-3-pyridyl]-2-isopropyl-pyrimidine-5-carboxamide

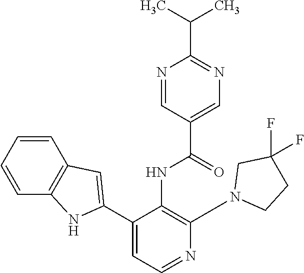 [SMILES: CC(C)c1ncc(C(=O)Nc2c(-c3cc4ccccc4[nH]3)ccnc2N2CCC(F)(F)C2)cn1]

### Step 1. Preparation of N-[4-[1-(benzenesulfonyl)indol-2-yl]-2-(3,3-difluoropyrrolidin-1-yl)-3-pyridyl]-2-isopropyl-pyrimidine-5-carboxamide

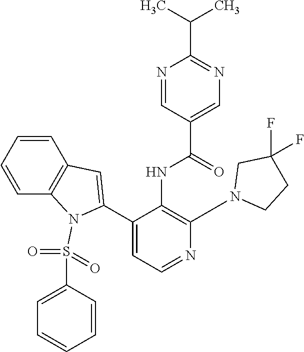 [SMILES: CC(C)c1ncc(C(=O)Nc2c(-c3cc4ccccc4n3S(=O)(=O)c3ccccc3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.10 g, 0.21 mmol), [1-(benzenesulfonyl)indol-2-yl]boronic acid (0.095 g, 0.32 mmol), and cesium carbonate (0.14 g, 0.42 mmol) in degassed 1,4-dioxane (2.4 mL) and water (0.28 mL) was added tetrakis(triphenylphosphine)palladium(0) (0.024 g, 0.021 mmol) and the mixture was stirred at 90° C. for 16 h. After cooling to ambient temperature, the mixture was diluted with ethyl acetate (20 mL). The mixture was passed through a bed of diatomaceous earth (i.e., Celite®). The solid was washed with ethyl acetate (30 mL) and the filtrate was concentrated in vacuo. The residue was used as is in the next step (0.11 g, 89%): MS (ESI+) m/z 603.4 (M+1).

### Step 2. Preparation of N-[2-(3,3-difluoropyrrolidin-1-yl)-4-(1H-indol-2-yl)-3-pyridyl]-2-isopropyl-pyrimidine-5-carboxamide

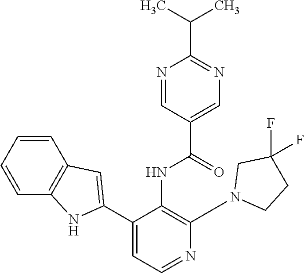 [SMILES: CC(C)c1ncc(C(=O)Nc2c(-c3cc4ccccc4[nH]3)ccnc2N2CCC(F)(F)C2)cn1]

To a solution of N-[4-[1-(benzenesulfonyl)indol-2-yl]-2-(3,3-difluoropyrrolidin-1-yl)-3-pyridyl]-2-isopropyl-pyrimidine-5-carboxamide (0.110 g, 0.18 mmol) in methanol (1.2 mL) was added cesium carbonate (0.21 g, 0.64 mmol). The mixture was stirred at 70° C. for 5 h. The reaction mixture was concentrated in vacuo and purified by preparative reverse-phase HPLC, eluting with a gradient of 5% to 35% of acetonitrile in water containing 0.5% formic acid, to afford the title compound as a colorless solid (0.039 g, 46% yield): ¹H-NMR (300 MHz; DMSO-d₆) δ 11.33 (s, 1H), 10.17 (s, 1H), 9.05 (s, 2H), 8.19 (d, J=5.1 Hz, 1H), 7.64 (d, J=7.9 Hz, 1H), 7.54 (d, J=2.6 Hz, 1H), 7.41 (d, J=8.0 Hz, 1H), 7.15-7.12 (m, 1H), 7.09-7.04 (m, 2H), 4.01-3.67 (m, 4H), 3.19 (dquintet, J=13.8, 6.9 Hz, 1H), 2.43 (td, J=14.1, 7.2 Hz, 2H), 1.29 (d, J=6.9 Hz, 6H); MS (ESI+) m/z 463.2 (M+1).

### Example 289

### Synthesis of 2-(3,3-difluoropyrrolidin-1-yl)-4-(2H-indazol-3-yl)-3-pyridyl]-2-isopropyl-pyrimidine-5-carboxamide

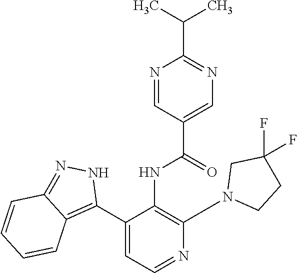 [SMILES: CC(C)c1ncc(C(=O)Nc2c(-c3[nH]nc4ccccc34)ccnc2N2CCC(F)(F)C2)cn1]

### Step 1. Preparation of potassium 2-(3,3-difluoropyrrolidin-1-yl)-4-iodo-pyridine-3-carboxylate

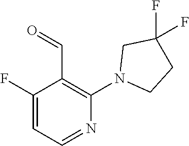 [SMILES: O=Cc1c(F)ccnc1N1CCC(F)(F)C1]

To a mixture of 2-fluoro-4-iodonicotinaldehyde (2.0 g, 8.0 mmol) and potassium carbonate (2.2 g, 16 mmol) in DMSO (30 mL) was added 3,3-difluoropyrrolidine hydrochloride (1.3 g, 8.8 mmol), and the mixture was stirred at 50° C. for 4 h. The mixture was partitioned between ethyl acetate (60 mL) and water. The layers were separated, and the organic layer was dried over anhydrous magnesium sulfate, filtered, and concentrated in vacuo. Purification of the residue by column chromatography, eluting with a gradient of 0-50% ethyl acetate in heptane, afforded the title compound as a yellow solid (2.4 g, 90% yield): ¹H NMR (300 MHz, DMSO-d₆) δ 10.02 (s, 1H), 7.90 (d, J=4.9 Hz, 1H), 7.39 (d, J=4.9 Hz, 1H), 3.65 (t, J=13.1 Hz, 2H), 3.56 (t, J=7.3 Hz, 2H), 2.56-2.42 (m, 2H); MS (ESI+) m/z 339.2 (M+1).

### Step 2. Preparation of 2-(3,3-difluoropyrrolidin-1-yl)-4-[1-(2-trimethylsilylethoxymethyl)indazol-3-yl]pyridine-3-carbaldehyde

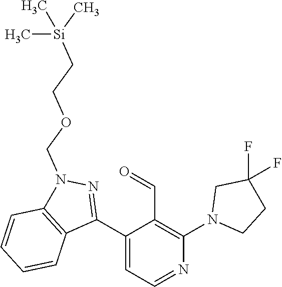 [SMILES: C[Si](C)(C)CCOCn1nc(-c2ccnc(N3CCC(F)(F)C3)c2C=O)c2ccccc21]

To a solution of 2-(indazol-1-yl-methoxy)ethyl-trimethyl-silane (0.35 g, 1.42 mmol) in THF (4.7325 mL) was added n-butyllithium (1.6M in hexane) (1.1 mL, 1.8 mmol) at −78° C., followed by the addition of zinc chloride (1M in diethyl ether, 2.7 mL, 2.7 mmol). After warming to the ambient temperature, stirring was continued for 30 min. To the mixture was added 2-(3,3-difluoropyrrolidin-1-yl)-4-iodo-pyridine-3-carbaldehyde (0.40 g, 1.2 mmol) and tetrakis(triphenylphosphine)palladium (0) (0.14 g, 0.12 mmol). The reaction mixture was stirred at 60° C. for 5 h. The mixture was cooled and partitioned between water and ethyl acetate, the organic layer was dried over anhydrous magnesium sulfate, filtered, and concentrated in vacuo. Purification of the residue by column chromatography, eluting with a gradient of 5-95% ethyl acetate in heptane, provide the title compound as a yellow solid (0.45 g, 83% yield): MS (ES+) m/z 459.2 (M+1).

### Step 3. Preparation of 2-(3,3-difluoropyrrolidin-1-yl)-4-[1-(2-trimethylsilylethoxymethyl)indazol-3-yl]pyridine-3-carboxylic acid; hydrochloride

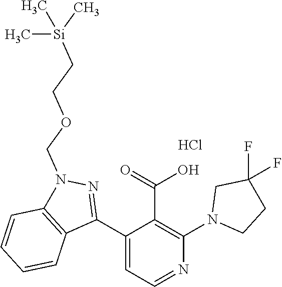 [SMILES: C[Si](C)(C)CCOCn1nc(-c2ccnc(N3CCC(F)(F)C3)c2C(=O)O)c2ccccc21 ; Cl]

To 2-(3,3-difluoropyrrolidin-1-yl)-4-[1-(2-trimethylsilylethoxymethyl)indazol-3-yl]pyridine-3-carbaldehyde (0.44 g, 0.96 mmol) was added tert-butanol (19 mL), dichloromethane (3.3 mL), and 2-methyl-2-butene (3.0 mL). The reaction mixture was cooled to 0° C. before a mixture of sodium dihydrogenphosphate (0.40 g, 3.3 mmol) and sodium chlorite (0.27 g, 2.4 mmol) in water (5.2 mL) was added dropwise. The reaction mixture was allowed to warm to ambient temperature and stirred for 18 h. The reaction mixture was diluted with ethyl acetate (20 mL) and washed with 1 M hydrochloric acid until the pH of the aqueous solution was <2. The aqueous layer was washed with ethyl acetate (3×40 mL), and the combined organic phases were washed with 1 M hydrochloric acid (2×40 mL) and brine (2×50 mL). The organic solution was dried over anhydrous magnesium sulfate, filtered, and concentrated in vacuo. The colorless oil was used without further purification (0.64 g, 100% yield): MS (ES+) m/z 475.3 (M+1).

### Step 4. Preparation of 2-(3,3-difluoropyrrolidin-1-yl)-4-[1-(2-trimethylsilylethoxymethyl)indazol-3-yl]pyridin-3-amine

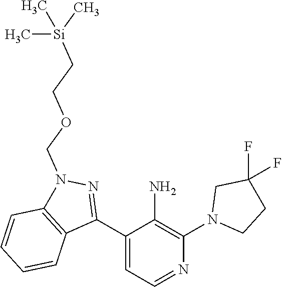 [SMILES: C[Si](C)(C)CCOCn1nc(-c2ccnc(N3CCC(F)(F)C3)c2N)c2ccccc21]

To 2-(3,3-difluoropyrrolidin-1-yl)-4-[1-(2-trimethylsilylethoxymethyl)indazol-3-yl]pyridine-3-carboxylic acid; hydrochloride (0.49 g, 1.0 mmol) was added N-methyl-2-pyrrolidone (10 mL), diphenylphosphonic azide (0.33 mL, 1.5 mmol), and triethylamine (0.36 mL, 2.6 mmol). The solution was heated at 95° C. for 2 h. The reaction mixture was cooled to ambient temperature and diluted with ethyl acetate (30 mL). The reaction mixture was washed with saturated ammonium chloride (30 mL), saturated sodium bicarbonate (2×30 mL), water (30 mL), and brine (30 mL). The organic layer was dried over anhydrous magnesium sulfate, filtered, and concentrated in vacuo. Purification of the residue by column chromatography, eluting with a gradient of 0-60% ethyl acetate in heptane, afforded the title compound as a colorless solid (0.21 g, 46% yield): MS (ES+) m/z 446.2 (M+1).

### Step 5. Preparation of N-[2-(3,3-difluoropyrrolidin-1-yl)-4-[1-(2-trimethylsilylethoxymethyl)indazol-3-yl]-3-pyridyl]-2-isopropyl-pyrimidine-5-carboxamide

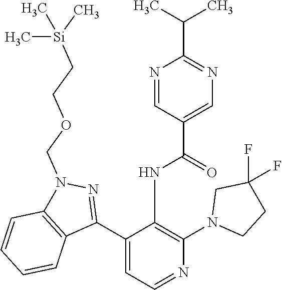 [SMILES: CC(C)c1ncc(C(=O)Nc2c(-c3nn(COCC[Si](C)(C)C)c4ccccc34)ccnc2N2CCC(F)(F)C2)cn1]

To a mixture of 2-(3,3-difluoropyrrolidin-1-yl)-4-[1-(2-trimethylsilylethoxymethyl)indazol-3-yl]pyridin-3-amine (0.21 g, 0.44 mmol), 2-isopropylpyrimidine-5-carboxylic acid (0.087 g, 0.52 mmol) and 2-chloro-1-methylpyridinium iodide (0.45 g, 1.7 mmol) and N,N-diisopropylethylamine (0.30 mL, 1.7 mmol) was added anhydrous tetrahydrofuran (5.5 mL). The reaction mixture was stirred at 65° C. for 16 h. The reaction mixture was cooled to ambient temperature and diluted with methanol (3 mL), and 1 M sodium hydroxide (2 mL). The mixture was stirred for 20 min at ambient temperature before it was diluted with ethyl acetate (10 mL). The organic layer was washed with saturated ammonium chloride solution (2×30 mL), dried over anhydrous magnesium sulfate, filtered, and concentrated in vacuo. Purification of the residue by column chromatography, eluting with a gradient of 0 to 60% of ethyl acetate in heptane, provided the title compound as a colorless solid (0.17 g, 64% yield): MS (ES+) m/z 594.4 (M+1).

### Step 6. Preparation of 2-(3,3-difluoropyrrolidin-1-yl)-4-(2H-indazol-3-yl)-3-pyridyl]-2-isopropyl-pyrimidine-5-carboxamide

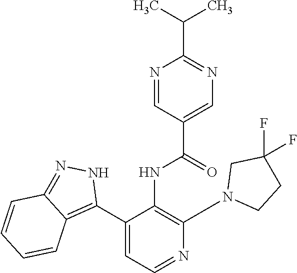 [SMILES: CC(C)c1ncc(C(=O)Nc2c(-c3[nH]nc4ccccc34)ccnc2N2CCC(F)(F)C2)cn1]

To N-[2-(3,3-difluoropyrrolidin-1-yl)-4-[1-(2-trimethylsilylethoxymethyl)indazol-3-yl]-3-pyridyl]-2-isopropyl-pyrimidine-5-carboxamide (0.17 g, 0.28 mmol) was added anhydrous dichloromethane (1.9 mL) and trifluoroacetic acid (1.9 mL) at ambient temperature. The solution was stirred at for 8 h. The reaction mixture was diluted with ethyl acetate (10 mL), washed with saturated sodium bicarbonate (3×20 mL), dried over anhydrous magnesium sulfate, filtered, and concentrated in vacuo. Purification of the residue by preparative reverse phase HPLC, eluting with a gradient of 5-90% acetonitrile in water containing 0.5% formic acid, provided the title compound as a colorless solid (0.49 g, 38% yield): ¹H-NMR (400 MHz; DMSO-d₆) δ 13.41 (s, 1H), 10.23 (s, 1H), 9.02 (s, 2H), 8.28 (d, J=5.0 Hz, 1H), 7.82 (d, J=8.2 Hz, 1H), 7.55 (d, J=8.4 Hz, 1H), 7.40-7.36 (m, 1H), 7.17 (q, J=4.9 Hz, 2H), 3.99-3.73 (m, 4H), 3.19 (7, J=6.9 Hz, 1H), 2.44 (td, J=14.2, 7.2 Hz, 2H), 1.29 (d, J=6.9 Hz, 6H); MS (ESI+) m/z 464.2 (M+1)

### Example 290

### Synthesis of N-[2-(3,3-difluoropyrrolidin-1-yl)-4-pyrimidin-2-yl-3-pyridyl]-2-isopropyl-pyrimidine-5-carboxamide

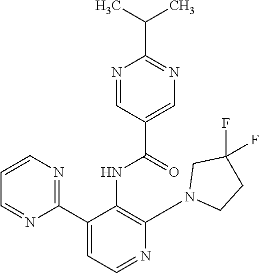 [SMILES: CC(C)c1ncc(C(=O)Nc2c(-c3ncccn3)ccnc2N2CCC(F)(F)C2)cn1]

To a solution of N-[2-(3,3-difluoropyrrolidin-1-yl)-4-iodo-3-pyridyl]-2-isopropyl-pyrimidine-5-carboxamide (0.080 g, 0.17 mmol), tetrakis(triphenylphosphine)palladium (0) (0.020 g, 0.017 mmol), copper(I) iodide (0.0032 g, 0.017 mmol) in 1,4-dioxane (1.2 mL) was added 2-(tributylstannyl)pyrimidine (0.094 g, 0.25 mmol) and stirred at 90° C. for 16 h. Upon completion, the mixture was cooled and partitioned between water and ethyl acetate. The organic layer was dried over anhydrous magnesium sulfate and concentrated in vacuo. Purification of the residue by preparative reverse phase HPLC, eluting with a gradient of 5-80% acetonitrile in water containing 0.5% formic acid, provided the title compound as a light yellow solid (0.026 g, 35% yield): ¹H-NMR (400 MHz; DMSO-d₆) δ 10.37 (s, 1H), 9.07 (s, 2H), 8.87 (d, J=4.9 Hz, 2H), 8.28 (d, J=5.0 Hz, 1H), 7.46 (t, J=4.9 Hz, 1H), 7.25 (d, J=5.0 Hz, 1H), 3.92 (t, J=13.4 Hz, 2H), 3.78 (t, J=7.2 Hz, 2H), 3.21 (dquintet, J=13.8, 6.9 Hz, 1H), 2.43 (td, J=14.2, 7.1 Hz, 2H), 1.30 (d, J=6.9 Hz, 6H); MS (ESI+) m/z 426.2 (M+1).

### Example 291

### Synthesis of N-[4-cyano-2-(3,3-difluoropyrrolidin-1-yl)-3-pyridyl]-2-isopropyl-pyrimidine-5-carboxamide

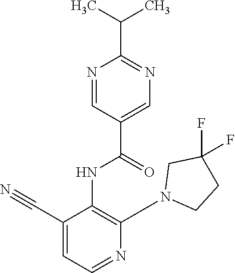 [SMILES: CC(C)c1ncc(C(=O)Nc2c(C#N)ccnc2N2CCC(F)(F)C2)cn1]

A solution of N-[2-(3,3-difluoropyrrolidin-1-yl)-4-iodo-3-pyridyl]-2-isopropyl-pyrimidine-5-carboxamide (0.10 g, 0.21 mmol), tetrakis(triphenylphosphine)palladium (0) (0.024 g, 0.021 mmol) and zinc cyanide (0.030 g, 0.25 mmol) in anhydrous N,N-dimethylformamide (1.4 mL) was stirred at 100° C. for 16 h under a nitrogen atmosphere. Upon completion, the mixture was cooled and partitioned between water and ethyl acetate. The organic layer was dried over anhydrous magnesium sulfate, filtered, and concentrated in vacuo. Purification of the residue by preparative reverse phase HPLC, eluting with a gradient of 5-80% acetonitrile in water containing 0.5% formic acid, provided the title compound as a colorless solid (0.017 g, 21% yield): ¹H-NMR (400 MHz; DMSO-d₆) δ 10.85 (s, 1H), 9.26 (s, 2H), 8.34 (d, J=4.9 Hz, 1H), 7.25 (d, J=4.9 Hz, 1H), 3.93 (t, J=13.1 Hz, 2H), 3.78 (t, J=7.3 Hz, 2H), 3.25 (dq, J=13.8, 6.9 Hz, 1H), 2.45 (dt, J=14.1, 7.1 Hz, 2H), 1.33 (d, J=6.9 Hz, 6H); MS (ESI-) m/z 371.2 (M−1).

### Example 292

### Synthesis of N-[2-(3,3-difluoropyrrolidin-1-yl)-4-tetrahydropyran-3-yl-3-pyridyl]-2-isopropyl-pyrimidine-5-carboxamide

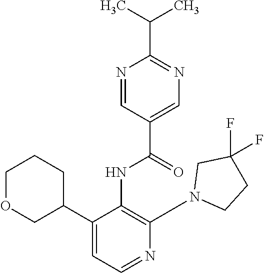 [SMILES: CC(C)c1ncc(C(=O)Nc2c(C3CCCOC3)ccnc2N2CCC(F)(F)C2)cn1]

To a mixture of N-(2-(3,3-difluoropyrrolidin-1-yl)-4-(3,4-dihydro-2H-pyran-6-yl)pyridin-3-yl)-2-isopropylpyrimidine-5-carboxamide (0.066 g, 0.15 mmol) in methanol (0.85 mL) and ethyl acetate (0.85 mL) was added ammonium formate (0.19 g, 3.07 mmol) and 10% palladium on carbon (0.025 g). The reaction mixture was stirred at 65° C. for 4 h. After cooling to ambient temperature, the mixture was diluted with ethyl acetate (20 mL), filtered through a bed of diatomaceous earth (i.e., Celite®), and concentrated in vacuo. The residue was purified by preparative reverse-phase HPLC, using a gradient of 5 to 75% acetonitrile in water containing 0.5% formic acid, to afford the title compound as a colorless solid (0.027 g, 40% yield): ¹H-NMR (400 MHz; DMSO-d₆): a 10.21 (s, 1H), 9.27 (s, 2H), 8.10 (d, J=5.1 Hz, 1H), 6.86 (d, J=5.2 Hz, 1H), 3.87-3.66 (m, 6H), 3.31-3.20 (m, 3H), 2.88-2.83 (m, 1H), 2.39 (tq, J=14.2, 7.1 Hz, 2H), 1.82-1.78 (m, 1H), 1.76-1.66 (m, 1H), 1.62-1.48 (m, 2H), 1.34 (d, J=6.9 Hz, 6H); MS (ESI+) m/z 432.2 (M+1).

In a similar manner as described in examples disclosed herein, utilizing the appropriately substituted starting materials and intermediates, the following compounds were prepared:

| Example | Structure | Amount | MS (ES+) | |
|---|---|---|---|---|
| No. | Name | Yield | m/z | ¹H NMR |
| 293 | 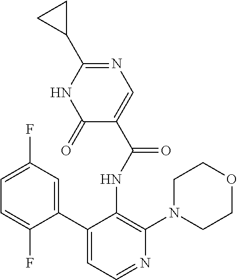 [SMILES: O=C(Nc1c(-c2cc(F)ccc2F)ccnc1N1CCOCC1)c1cnc(C2CC2)[nH]c1=O] 2-cyclopropyl-N-(4-(2,5-difluorophenyl)-2- morpholinopyridin-3-yl)-6-oxo-1,6- dihydropyrimidine-5-carboxamide | 0.087 g 28% | 454.2 (M + 1) | (300 MHz, DMSO-d₆) δ 13.69 (s, 0.4H), 10.81 (s, 1H), 8.30 (s, 1H), 8.25 (d, J = 5.0 Hz, 1H), 7.31-7.15 (m, 3H), 7.04 (dd, J = 5.0, 0.8 Hz, 1H), 3.77-3.59 (m, 4H), 3.19-3.00 (m, 4H), 2.04-1.96 (m, 1H), 1.15- 1.09 (m, 4H) |

### Example 294

### 2-isopropyl-N-(4-phenyl-2-(6-(2,2,2-trifluoroethyl)-2,6-diazaspiro[3.3]heptan-2-yl)pyridin-3-yl)pyrimidine-5-carboxamide

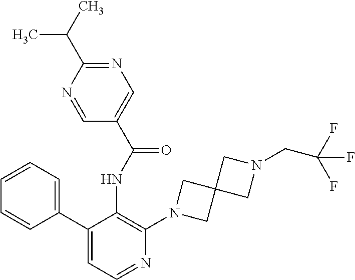 [SMILES: CC(C)c1ncc(C(=O)Nc2c(-c3ccccc3)ccnc2N2CC3(CN(CC(F)(F)F)C3)C2)cn1]

### Step 1. Preparation of tert-butyl 6-(3-nitro-4-phenylpyridin-2-yl)-2,6-diazaspiro[3.3]heptane-2-carboxylate

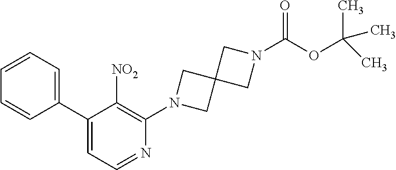 [SMILES: CC(C)(C)OC(=O)N1CC2(C1)CN(c1nccc(-c3ccccc3)c1[N+](=O)[O-])C2]

To a mixture of 2-chloro-3-nitro-4-phenylpyridine (0.810 g, 3.45 mmol) in anhydrous N-methyl-2-pyrrolidone (40.0 mL) was added N,N-diisopropylethylamine (6.20 mL, 34.5 mmol) and tert-butyl 2,6-diazaspiro[3.3]heptane-2-carboxylate hydrochloride (1.22 g, 5.18 mmol). The reaction was stirred at 100° C. for 4 h. After cooling to ambient temperature, the mixture was diluted with ethyl acetate (300 mL) and the organic phase was washed with saturated ammonium chloride (100 mL), water (4×100 mL), and brine (100 mL), then dried over anhydrous magnesium sulfate, filtered and concentrated in vacuo. The residue was purified by column chromatography, eluting with a gradient of 15 to 100% ethyl acetate in heptane, to afford the title compound as a yellow solid (1.129 g, 79% yield): MS (ES+) m/z 397.2 (M+1).

### Step 2. Preparation of 2-(3-nitro-4-phenylpyridin-2-yl)-2,6-diazaspiro[3.3]heptane trifluoroacetic acid salt

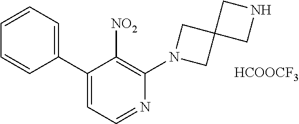

To a mixture of tert-butyl 6-(3-nitro-4-phenylpyridin-2-yl)-2,6-diazaspiro[3.3]heptane-2-carboxylate (1.13 g, 2.73 mmol) in anhydrous dichloromethane (14 mL) was added trifluoroacetic acid (3.14 mL, 41.0 mmol). The reaction was stirred at 40° C. for 2 h. After cooling to ambient temperature, the mixture was concentrated in vacuo to afford the title compound as a crude yellow solid (1.44 g, 128% yield): MS (ES+) m/z 297.2 (M+1).

### Step 3. Preparation of 2-(3-nitro-4-phenylpyridin-2-yl)-6-(2,2,2-trifluoroethyl)-2,6-diazaspiro[3.3]heptane

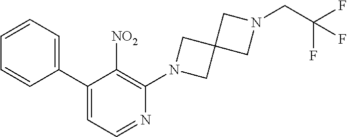 [SMILES: O=[N+]([O-])c1c(-c2ccccc2)ccnc1N1CC2(CN(CC(F)(F)F)C2)C1]

To a mixture of 2-(3-nitro-4-phenylpyridin-2-yl)-2,6-diazaspiro[3.3]heptane trifluoroacetic acid salt (0.300 g, 0.731 mmol) and N,N-diisopropylethylamine (2.55 mL, 14.6 mmol) in anhydrous tetrahydrofuran (7.30 mL) at 0° C. was added 2,2,2-trifluoroethyl trifluoromethanesulfonate (0.32 mL, 2.2 mmol). The reaction mixture was stirred at ambient temperature for 16 h. The mixture was diluted with ethyl acetate (100 mL) and the organic phase was washed with water (30 mL) and brine (30 mL), dried over anhydrous magnesium sulfate, filtered, and concentrated in vacuo to afford the title compound as a yellow solid (0.249 g, 90% yield): MS (ES+) m/z 379.2 (M+1).

### Step 4. Preparation of 4-phenyl-2-(6-(2,2,2-trifluoroethyl)-2,6-diazaspiro[3.3]heptan-2-yl)pyridin-3-amine

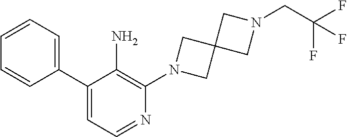 [SMILES: Nc1c(-c2ccccc2)ccnc1N1CC2(CN(CC(F)(F)F)C2)C1]

A mixture of 2-(3-nitro-4-phenylpyridin-2-yl)-6-(2,2,2-trifluoroethyl)-2,6-diazaspiro[3.3]heptane (0.249 g, 0.657 mmol) in methanol (15 mL) and ethyl acetate (15 mL) was degassed with nitrogen for 10 minutes. To the reaction mixture was added ammonium formate (2.49 g, 39.4 mmol) and 10% palladium on carbon (0.185 g). The reaction was stirred at 65° C. for 1 h. After cooling to ambient temperature, the mixture was diluted in 150 mL of ethyl acetate, filtered through a bed of diatomaceous earth (i.e., Celite®) and concentrated in vacuo, to afford the title compound as a crude red oil (0.249 g, 109% yield): MS (ES+) m/z 349.2 (M+1).

### Step 5. Preparation of 2-isopropyl-N-(4-phenyl-2-(6-(2,2,2-trifluoroethyl)-2,6-diazaspiro[3.3]heptan-2-yl)pyridin-3-yl)pyrimidine-5-carboxamide

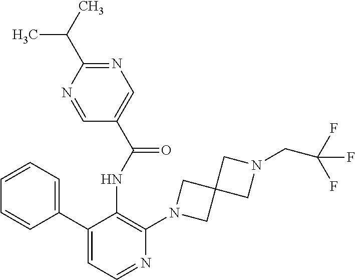 [SMILES: CC(C)c1ncc(C(=O)Nc2c(-c3ccccc3)ccnc2N2CC3(CN(CC(F)(F)F)C3)C2)cn1]

To a mixture of 4-phenyl-2-(6-(2,2,2-trifluoroethyl)-2,6-diazaspiro[3.3]heptan-2-yl)pyridin-3-amine (0.085 g, 0.24 mmol) in anhydrous tetrahydrofuran (2.4 mL) was added N,N-diisopropylethylamine (0.85 mL, 4.9 mmol), 2-chloro-1-methylpyridinium iodide (0.187 g, 0.732 mmol), and 2-isopropylpyrimidine-5-carboxylic acid (0.049 g, 0.29 mmol). The reaction mixture was stirred at 65° C. for 4 h. After cooling to ambient temperature, the mixture was diluted with ethyl acetate (150 mL), filtered through a bed of diatomaceous earth (i.e., Celite®) and concentrated in vacuo. Purification of the residue by reverse-phase column chromatography, eluting with a gradient of 10 to 100% of acetonitrile in water containing 0.5% formic acid, afforded the title compound as a colorless oil. Further purification of the residue by reverse-phase column chromatography, using a gradient of 0 to 100% of acetonitrile in water containing 0.5% formic acid as eluent, afforded the title compound as a colorless solid (0.028 g, 23% yield): ¹H NMR (300 MHz, DMSO-d₆) δ 10.05 (s, 1H), 8.99 (s, 2H), 8.12 (d, J=5.0 Hz, 1H), 7.38-7.29 (m, 5H), 6.71 (d, J=5.1 Hz, 1H), 4.19-4.02 (m, 4H), 3.47 (s, 4H), 3.16 (m, 3H), 1.28 (d, J=6.9 Hz, 6H); MS (ES+) 497.2 m/z (M+1).

### Example 295

### Synthesis of N-(2-(6-acetyl-2,6-diazaspiro[3.3]heptan-2-yl)-4-phenylpyridin-3-yl)-2-isopropylpyrimidine-5-carboxamide

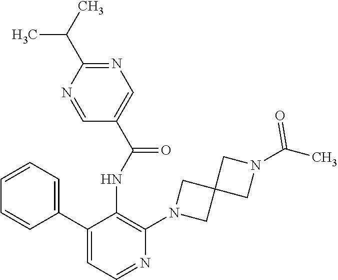 [SMILES: CC(=O)N1CC2(C1)CN(c1nccc(-c3ccccc3)c1NC(=O)c1cnc(C(C)C)nc1)C2]

### Step 1. Preparation of 1-(6-(3-nitro-4-phenylpyridin-2-yl)-2,6-diazaspiro[3.3]heptan-2-yl)ethan-1-one

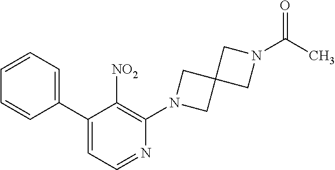 [SMILES: CC(=O)N1CC2(C1)CN(c1nccc(-c3ccccc3)c1[N+](=O)[O-])C2]

To a mixture of 2-(3-nitro-4-phenylpyridin-2-yl)-2,6-diazaspiro[3.3]heptane trifluoroacetic acid salt (0.300 g, 0.731 mmol) and N,N-diisopropylethylamine (2.55 mL, 14.6 mmol) in anhydrous tetrahydrofuran (7.30 mL) at 0° C. was added acetyl chloride (0.16 mL, 2.2 mmol). The reaction mixture was stirred at ambient temperature for 2.5 h. The mixture was diluted with ethyl acetate (100 mL) and the organic phase was washed with water (30 mL) and brine (30 mL), dried over anhydrous magnesium sulfate, filtered, and concentrated in vacuo. The residue was purified by column chromatography, eluting with a gradient of 0 to 50% methanol in ethyl acetate, to afford the title compound as an orange solid (0.196 g, 79% yield): MS (ES+) m/z 339.2 (M+1).

### Step 2. Preparation of 1-(6-(3-amino-4-phenylpyridin-2-yl)-2,6-diazaspiro[3.3]heptan-2-yl)ethan-1-one

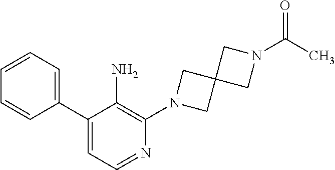 [SMILES: CC(=O)N1CC2(C1)CN(c1nccc(-c3ccccc3)c1N)C2]

A mixture of 1-(6-(3-nitro-4-phenylpyridin-2-yl)-2,6-diazaspiro[3.3]heptan-2-yl)ethan-1-one (0.196 g, 0.579 mmol) in methanol (15 mL) and ethyl acetate (15 mL) was degassed with nitrogen for 10 minutes. To the reaction mixture was added ammonium formate (1.10 g, 17.4 mmol) and 10% palladium on carbon (0.100 g). The reaction was stirred at 65° C. for 1 h. After cooling to ambient temperature, the mixture was diluted in 150 mL of ethyl acetate, filtered through a bed of diatomaceous earth (i.e., Celite®) and concentrated in vacuo, to afford the title compound as a crude brown solid (0.178 g, 100% yield): MS (ES+) m/z 309.2 (M+1).

### Step 3. Preparation of N-(2-(6-acetyl-2,6-diazaspiro[3.3]heptan-2-yl)-4-phenylpyridin-3-yl)-2-isopropylpyrimidine-5-carboxamide

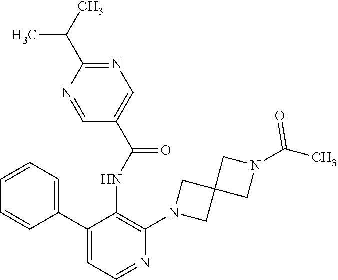 [SMILES: CC(=O)N1CC2(C1)CN(c1nccc(-c3ccccc3)c1NC(=O)c1cnc(C(C)C)nc1)C2]

To a mixture of 1-(6-(3-amino-4-phenylpyridin-2-yl)-2,6-diazaspiro[3.3]heptan-2-yl)ethan-1-one (0.101 g, 0.328 mmol) in anhydrous tetrahydrofuran (6.6 mL) was added N,N-diisopropylethylamine (0.57 mL, 3.3 mmol), 2-chloro-1-methylpyridinium iodide (0.251 g, 0.983 mmol), and 2-isopropylpyrimidine-5-carboxylic acid (0.065 g, 0.39 mmol). The reaction mixture was stirred at 65° C. for 2 h. After cooling to ambient temperature, the mixture was diluted in ethyl acetate (150 mL), filtered through a bed of diatomaceous earth (i.e., Celite®) and concentrated in vacuo. Purification of the residue by reverse-phase column chromatography, using a gradient of 0 to 100% of acetonitrile in water containing 0.5% formic acid as eluent, afforded the title compound as a colorless solid (0.084 g, 56% yield): ¹H NMR (300 MHz, DMSO-d₆) δ 10.08 (s, 1H), 8.98 (s, 2H), 8.13 (d, J=5.0 Hz, 1H), 7.40-7.30 (m, 5H), 6.73 (d, J=5.1 Hz, 1H), 4.24-4.07 (m, 6H), 3.96 (s, 2H), 3.18 (sept, J=6.9 Hz, 1H), 1.70 (s, 3H), 1.28 (d, J=6.9 Hz, 6H); 457.2 m/z (M+1).

### Example 296

### Synthesis of N-(2-(3-fluoro-3-methylpyrrolidin-1-yl)-4-phenylpyridin-3-yl)-2-isopropylpyrimidine-5-carboxamide

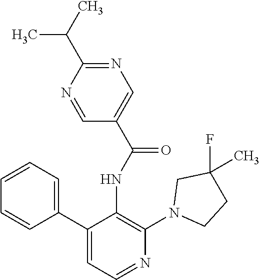 [SMILES: CC(C)c1ncc(C(=O)Nc2c(-c3ccccc3)ccnc2N2CCC(C)(F)C2)cn1]

### Step 1. Preparation of 2-(3-fluoro-3-methylpyrrolidin-1-yl)-3-nitro-4-phenylpyridine

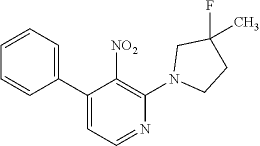 [SMILES: CC1(F)CCN(c2nccc(-c3ccccc3)c2[N+](=O)[O-])C1]

To a mixture of 2-chloro-3-nitro-4-phenylpyridine (0.504 g, 2.10 mmol) in anhydrous N-methyl-2-pyrrolidone (18.0 mL) was added N,N-diisopropylethylamine (3.10 mL, 17.9 mmol) and 3-fluoro-3-methylpyrrolidine hydrochloride (0.250 g, 1.79 mmol). The reaction was stirred at 80° C. for 16 h. After cooling to ambient temperature, the mixture was diluted in ethyl acetate (100 mL) and the organic phase was washed with saturated ammonium chloride (30 mL), water (4×30 mL), and brine (30 mL), then dried over anhydrous magnesium sulfate, filtered, and concentrated in vacuo. The residue was purified by column chromatography, eluting with a gradient of 0 to 65% ethyl acetate in heptane, to afford the title compound as a yellow oil (0.623 g, 92% yield): MS (ES+) m/z 302.2 (M+1).

### Step 2. Preparation of 2-(3-fluoro-3-methylpyrrolidin-1-yl)-4-phenylpyridin-3-amine

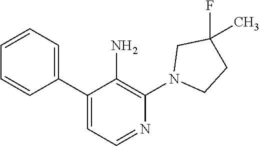 [SMILES: CC1(F)CCN(c2nccc(-c3ccccc3)c2N)C1]

A mixture of 2-(3-fluoro-3-methylpyrrolidin-1-yl)-3-nitro-4-phenylpyridine (0.623 g, 1.65 mmol) in methanol (5.5 mL) and ethyl acetate (5.5 mL) was degassed with nitrogen for 10 minutes. To the reaction mixture was added ammonium formate (3.13 g, 49.6 mmol) and 10% palladium on carbon (0.088 g). The reaction was stirred at 65° C. for 1 h. After cooling to ambient temperature, the mixture was diluted in 150 mL of ethyl acetate, filtered through a bed of diatomaceous earth (i.e., Celite®) and concentrated in vacuo. The residue was purified by column chromatography, eluting with a gradient of 0 to 100% ethyl acetate in heptane, to afford the title compound as a yellow oil (0.409 g, 91% yield): MS (ES+) m/z 272.2 (M+1).

### Step 3. Preparation of N-(2-(3-fluoro-3-methylpyrrolidin-1-yl)-4-phenylpyridin-3-yl)-2-isopropylpyrimidine-5-carboxamide

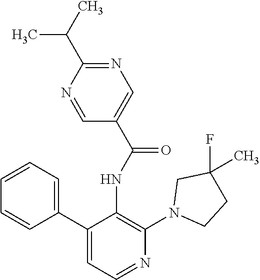 [SMILES: CC(C)c1ncc(C(=O)Nc2c(-c3ccccc3)ccnc2N2CCC(C)(F)C2)cn1]

To a mixture of 2-(3-fluoro-3-methylpyrrolidin-1-yl)-4-phenylpyridin-3-amine (0.104 g, 0.383 mmol) in anhydrous tetrahydrofuran (3.8 mL) was added N,N-diisopropylethylamine (1.3 mL, 7.7 mmol), 2-chloro-1-methylpyridinium iodide (0.294 g, 1.15 mmol), and 2-isopropylpyrimidine-5-carboxylic acid (0.076 g, 0.46 mmol). The reaction was stirred at 65° C. for 4 h. After cooling to ambient temperature, the mixture was diluted in ethyl acetate (100 mL) and the organic phase was washed with saturated ammonium chloride (2×30 mL) and brine (30 mL), dried over anhydrous magnesium sulfate, filtered, and concentrated in vacuo. Purification of the residue by reverse-phase column chromatography, using a gradient of 0 to 100% acetonitrile in water containing 0.5% formic acid as eluent, afforded the title compound as a colorless solid (0.085 g, 53% yield): ¹H NMR (300 MHz, DMSO-d₆) δ 10.19-10.10 (m, 1H), 8.91 (s, 2H), 8.13 (d, J=5.0 Hz, 1H), 7.42-7.26 (m, 5H), 6.68 (d, J=5.0 Hz, 1H), 3.76-3.62 (m, 4H), 3.15 (sept, J=6.9 Hz, 1H), 2.18-1.88 (m, 2H), 1.50 (d, J=20.9 Hz, 3H), 1.26 (d, J=6.9 Hz, 6H); MS (ES+) 420.2 m/z (M+1).

### Example 297

### Synthesis of N-(2-((3S,4R)-3,4-difluoropyrrolidin-1-yl)-4-(2-fluorophenyl)pyridin-3-yl)-2-isopropylpyrimidine-5-carboxamide

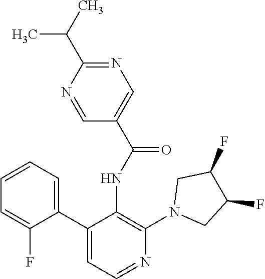 [SMILES: CC(C)c1ncc(C(=O)Nc2c(-c3ccccc3F)ccnc2N2C[C@@H](F)[C@@H](F)C2)cn1]

### Step 1. Preparation of 2-((3S,4R)-3,4-difluoropyrrolidin-1-yl)-4-(2-fluorophenyl)-3-nitropyridine

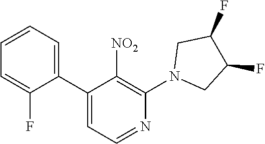 [SMILES: O=[N+]([O-])c1c(-c2ccccc2F)ccnc1N1C[C@@H](F)[C@@H](F)C1]

To a mixture of 2-chloro-4-(2-fluorophenyl)-3-nitro-pyridine (0.800 g, 3.17 mmol) in anhydrous N-methyl-2-pyrrolidone (16.0 mL) was added N,N-diisopropylethylamine (5.70 mL, 31.7 mmol) and (3R,4S)-3,4-difluoropyrrolidine hydrochloride (0.682 g, 4.75 mmol). The reaction was stirred at 50° C. for 16 h. After cooling to ambient temperature, the mixture was diluted in ethyl acetate (100 mL) and the organic phase was washed with saturated ammonium chloride (30 mL), water (4×30 mL), brine (30 mL), dried over anhydrous magnesium sulfate, filtered and concentrated in vacuo. The residue was purified by column chromatography, eluting with a gradient of 0 to 35% ethyl acetate in heptane, to afford the title compound as a red oil (0.958 g, 94% yield): MS (ES+) m/z 324.0 (M+1).

### Step 2. 2-((3S,4R)-3,4-difluoropyrrolidin-1-yl)-4-(2-fluorophenyl)pyridin-3-amine

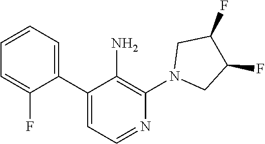 [SMILES: Nc1c(-c2ccccc2F)ccnc1N1C[C@@H](F)[C@@H](F)C1]

A mixture of 2-((3S,4R)-3,4-difluoropyrrolidin-1-yl)-4-(2-fluorophenyl)-3-nitropyridine (0.958 g, 2.96 mmol) in methanol (10 mL) and ethyl acetate (10 mL) was degassed with nitrogen for 10 minutes. To the reaction mixture was added ammonium formate (5.61 g, 88.9 mmol), 10% palladium on carbon (0.095 g). The reaction was stirred at 65° C. for 1.5 h. After cooling to ambient temperature, the mixture was diluted in 150 mL of ethyl acetate, filtered through a bed of diatomaceous earth (i.e., Celite®) and concentrated in vacuo. The residue was purified by column chromatography, eluting with a gradient of 15 to 65% ethyl acetate in heptane, to afford the title compound as a pink oil (0.801 g, 92% yield): MS (ES+) m/z 294.2 (M+1).

### Step 3. Preparation of 2-((3S,4R)-3,4-difluoropyrrolidin-1-yl)-4-(2-fluorophenyl)pyridin-3-amine

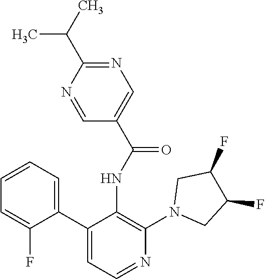 [SMILES: CC(C)c1ncc(C(=O)Nc2c(-c3ccccc3F)ccnc2N2C[C@@H](F)[C@@H](F)C2)cn1]

To a mixture of 2-((3S,4R)-3,4-difluoropyrrolidin-1-yl)-4-(2-fluorophenyl)pyridin-3-amine (0.083 g, 0.283 mmol) in anhydrous tetrahydrofuran (2.8 mL) was added N,N-diisopropylethylamine (0.50 mL, 2.8 mmol), 2-chloro-1-methylpyridinium iodide (0.217 g, 0.849 mmol), and 2-isopropylpyrimidine-5-carboxylic acid (0.056 g, 0.34 mmol). The reaction mixture was stirred at 65° C. for 3 h. After cooling to ambient temperature, the mixture was diluted in ethyl acetate (100 mL) and the organic phase was washed with saturated ammonium chloride (2×30 mL) and brine (30 mL), dried over anhydrous magnesium sulfate, filtered, and concentrated in vacuo. Purification of the residue by reverse-phase column chromatography, using a gradient of 0 to 100% acetonitrile in water containing 0.5% formic acid as eluent, afforded the title compound as a colorless solid (0.060 g, 48% yield): ¹H NMR (300 MHz, DMSO-d₆) δ 10.20 (s, 1H), 8.88 (s, 2H), 8.19 (d, J=5.0 Hz, 1H), 7.41-7.16 (m, 4H), 6.79 (dd, J=5.0, 0.7 Hz, 1H), 5.47-5.22 (m, 2H), 3.94-3.65 (m, 4H), 3.16 (sept, J=6.9 Hz, 1H), 1.26 (d, J=6.9 Hz, 6H); 442.2 m/z (M+1).

### Examples 298 and 299

In a similar manner as described in examples disclosed herein, utilizing the appropriately substituted starting materials and intermediates, the following compounds were prepared:

| Example | Structure | | MS (ES+) | |
|---|---|---|---|---|
| No. | Name | Amount | m/z | ¹H NMR |
| 298 | 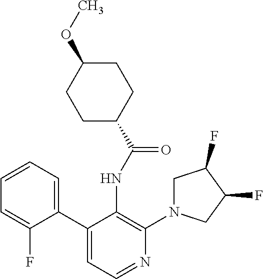 [SMILES: CO[C@H]1CC[C@H](C(=O)Nc2c(-c3ccccc3F)ccnc2N2C[C@@H](F)[C@@H](F)C2)CC1] (1r,4R)-N-(2-((3S,4R)-3,4- difluoropyrrolidin-1-yl)-4-(2- fluorophenyl)pyridin-3-yl)-4- methoxycyclohexane-1- carboxamide | 0.056 g 46% | 434.2 (M + 1) | (300 MHz, DMSO-d6) δ 9.23 (s, 1H), 8.10 (d, J = 4.9 Hz, 1H), 7.46-7.38 (m, 1H), 7.28-7.15 (m, 3H), 6.67 (dd, J = 5.0, 0.5 Hz, 1H), 5.48-5.41 (m, 1H), 5.31-5.24 (m, 1H), 3.95-3.60 (m, 4H), 3.18 (s, 3H), 2.98-2.89 (m, 1H), 2.06-1.97 (m, 1H), 1.95-1.85 (m, 2H), 1.61-0.86 (m, 6H) |
| 299 | 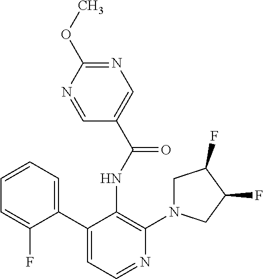 [SMILES: COc1ncc(C(=O)Nc2c(-c3ccccc3F)ccnc2N2C[C@@H](F)[C@@H](F)C2)cn1] N-(2-((3S,4R)-3,4- difluoropyrrolidin-1-yl)-4-(2- fluorophenyl)pyridin-3-yl)-2- methoxypyrimidine-5- carboxamide | 0.043 g 55% | 430.2 (M + 1) | (300 MHz, DMSO-de) δ 10.06 (s, 1H), 8.81 (d, J = 1.4 Hz, 2H), 8.18 (dd, J = 4.9, 1.3 Hz, 1H), 7.38-7.15 (m, 4H), 6.78 (d, J = 4.7 Hz, 1H), 5.42- 5.25 (m, 2H), 3.79 (m, 7H) |

### Example 300

### Synthesis of N-(2-((3S,4R)-3,4-difluoropyrrolidin-1-yl)-4-(2-fluorophenyl)pyridin-3-yl)-2-isopropylpyriinidine-5-carboxamide

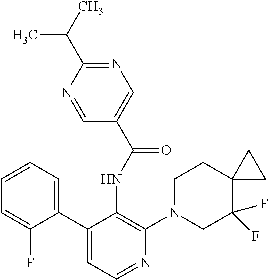 [SMILES: CC(C)c1ncc(C(=O)Nc2c(-c3ccccc3F)ccnc2N2CCC3(CC3)C(F)(F)C2)cn1]

### Step 1. Preparation of 4,4-difluoro-6-(4-(2-fluorophenyl)-3-nitropyridin-2-yl)-6-azaspiro[2.5]octane

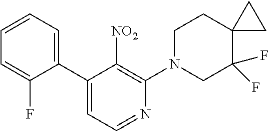 [SMILES: O=[N+]([O-])c1c(-c2ccccc2F)ccnc1N1CCC2(CC2)C(F)(F)C1]

To a mixture of 2-chloro-4-(2-fluorophenyl)-3-nitro-pyridine (0.358 g, 1.42 mmol) in anhydrous N,N-dimethylformamide (5.5 mL) was added N,N-diisopropylethylamine (2.0 mL, 11 mmol) and 4,4-difluoro-6-azaspiro[2.5]octane hydrochloride (0.200 g, 1.09 mmol). The reaction was stirred at 50° C. for 16 h, followed by 80° C. for 5 h. After cooling to ambient temperature, the mixture was diluted in ethyl acetate (100 mL) and the organic phase was washed with saturated ammonium chloride (30 mL), water (4×30 mL), brine (30 mL), dried over anhydrous magnesium sulfate, filtered and concentrated in vacuo. The residue was purified by column chromatography, eluting with a gradient of 0 to 65% ethyl acetate in heptane, to afford the title compound as a yellow oil (0.437 g, 111% yield): MS (ES+) m/z 364.3 (M+1).

### Step 2. Preparation of 2-(4,4-difluoro-6-azaspiro[2.5]octan-6-yl)-4-(2-fluorophenyl)pyridin-3-amine

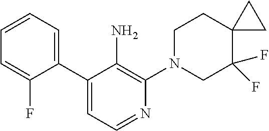 [SMILES: Nc1c(-c2ccccc2F)ccnc1N1CCC2(CC2)C(F)(F)C1]

A mixture of 4,4-difluoro-6-(4-(2-fluorophenyl)-3-nitropyridin-2-yl)-6-azaspiro[2.5]octane (0.437 g, 1.20 mmol) in methanol (5.5 mL) and ethyl acetate (5.5 mL) was degassed with nitrogen for 10 minutes. To the reaction mixture was added ammonium formate (1.37 g, 21.8 mmol), 10% palladium on carbon (0.116 g). The reaction was stirred at 65° C. for 0.5 h then ambient temperature for 16 h. The mixture was diluted in 100 mL of ethyl acetate, filtered through a bed of diatomaceous earth (i.e., Celite®) and concentrated in vacuo. The residue was purified by column chromatography, eluting with a gradient of 5 to 75% ethyl acetate in heptane, to afford the title compound as a pink oil (0.105 g, 29% yield): MS (ES+) m/z 334.3 (M+1).

### Step 3. Preparation of N-(2-((3S,4R)-3,4-difluoropyrrolidin-1-yl)-4-(2-fluorophenyl)pyridin-3-yl)-2-isopropylpyrimidine-5-carboxamide

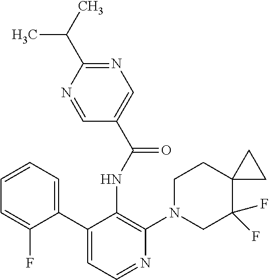 [SMILES: CC(C)c1ncc(C(=O)Nc2c(-c3ccccc3F)ccnc2N2CCC3(CC3)C(F)(F)C2)cn1]

To a mixture of 2-(4,4-difluoro-6-azaspiro[2.5]octan-6-yl)-4-(2-fluorophenyl)pyridin-3-amine (0.105 g, 0.314 mmol) in anhydrous tetrahydrofuran (6.3 mL) was added N,N-diisopropylethylamine (0.55 mL, 3.1 mmol), 2-chloro-1-methylpyridinium iodide (0.241 g, 0.943 mmol), and 2-isopropylpyrimidine-5-carboxylic acid (0.057 g, 0.35 mmol). The reaction mixture was stirred at 65° C. for 16 h. After cooling to ambient temperature, the mixture was diluted in ethyl acetate (150 mL) and the organic phase was washed with saturated ammonium chloride (2×50 mL) and brine (50 mL), dried over anhydrous magnesium sulfate, filtered, and concentrated in vacuo. Purification of the residue by column chromatography, using a gradient of 15 to 100% ethyl acetate in heptane as eluent, afforded the title compound as a colorless solid (0.090 g, 60% yield): ¹H NMR (400 MHz, DMSO-d₆) δ 10.20 (s, 1H), 8.90 (s, 2H), 8.31 (d, J=5.0 Hz, 1H), 7.41-7.33 (m, 2H), 7.24 (dt, J=26.6, 8.2 Hz, 2H), 7.08 (dd, J=5.0, 0.7 Hz, 1H), 3.54 (t, J=11.2 Hz, 2H), 3.37-3.35 (m, 2H), 3.17 (sept, J=6.9 Hz, 1H), 1.68-1.66 (m, 2H), 1.26 (d, J=6.9 Hz, 6H), 0.80-0.77 (m, 2H), 0.52-0.50 (m, 2H); ¹⁹F NMR (376 MHz, DMSO-d₆) δ -110.1, -114.7; MS (ES+) m/z 482.2 (M+1).

### Example 301

### Synthesis of N-(2-(1,1-difluoro-5-azaspiro[2.3]hexan-5-yl)-4-(2-fluorophenyl)pyridin-3-yl)-2-isopropylpyrimidine-5-carboxamide

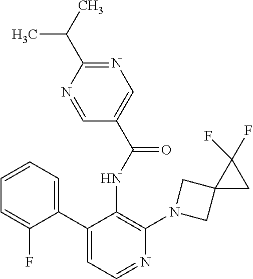 [SMILES: CC(C)c1ncc(C(=O)Nc2c(-c3ccccc3F)ccnc2N2CC3(C2)CC3(F)F)cn1]

### Step 1. Preparation of 1,1-difluoro-5-(4-(2-fluorophenyl)-3-nitropyridin-2-yl)-5-azaspiro[2.3]hexane

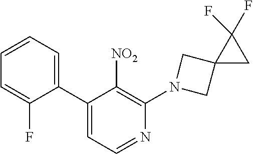 [SMILES: O=[N+]([O-])c1c(-c2ccccc2F)ccnc1N1CC2(C1)CC2(F)F]

To a mixture of 2-chloro-4-(2-fluorophenyl)-3-nitro-pyridine (0.082 g, 0.32 mmol) in anhydrous N-methyl-2-pyrrolidone (4.0 mL) was added N,N-diisopropylethylamine (0.37 mL, 2.1 mmol) and 4,4-difluoro-5-azaspiro[2.3]hexane hydrochloride (0.032 g, 0.21 mmol). The reaction mixture was stirred at 50° C. for 24 h. After cooling to ambient temperature, the mixture was diluted in ethyl acetate (100 mL) and the organic phase was washed with saturated ammonium chloride (2×30 mL) and brine (30 mL), dried over anhydrous magnesium sulfate, filtered and concentrated in vacuo. The residue was purified by column chromatography, eluting with a gradient of 0 to 100% ethyl acetate in heptane, to afford the title compound as a colorless oil (0.071 g, 97% yield): MS (ES+) m/z 336.0 (M+1).

### Step 2. Preparation of 2-(1,1-difluoro-5-azaspiro[2.3]hexan-5-yl)-4-(2-fluorophenyl)pyridin-3-amine

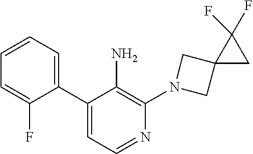 [SMILES: Nc1c(-c2ccccc2F)ccnc1N1CC2(C1)CC2(F)F]

A mixture of 1,1-difluoro-5-(4-(2-fluorophenyl)-3-nitropyridin-2-yl)-5-azaspiro[2.3]hexane (0.071 g, 0.21 mmol) in methanol (2.1 mL) and ethyl acetate (2.1 mL) was degassed with nitrogen for 10 minutes. To the reaction mixture was added 10% palladium on carbon (0.025 g) and the reaction mixture was stirred under hydrogen atmosphere and ambient temperature for 1 h. The mixture was diluted in 100 mL of ethyl acetate, filtered through a bed of diatomaceous earth (i.e., Celite®) and concentrated in vacuo. The residue was purified by column chromatography, eluting with a gradient of 5 to 75% ethyl acetate in heptane, to afford the title compound as a yellow oil (0.029 g, 45% yield): MS (ES+) m/z 306.0 (M+1).

### Step 3. Preparation of N-(2-(1,1-difluoro-5-azaspiro[2.3]hexan-5-yl)-4-(2-fluorophenyl)pyridin-3-yl)-2-isopropylpyrimidine-5-carboxamide

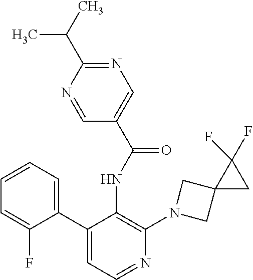 [SMILES: CC(C)c1ncc(C(=O)Nc2c(-c3ccccc3F)ccnc2N2CC3(C2)CC3(F)F)cn1]

To a mixture of 2-(1,1-difluoro-5-azaspiro[2.3]hexan-5-yl)-4-(2-fluorophenyl)pyridin-3-amine (0.029 g, 0.093 mmol) in anhydrous tetrahydrofuran (2.0 mL) was added N,N-diisopropylethylamine (0.16 mL, 0.93 mmol), 2-chloro-1-methylpyridinium iodide (0.048 g, 0.19 mmol), and 2-isopropylpyrimidine-5-carboxylic acid (0.016 g, 0.09 mmol). The reaction mixture was stirred at 65° C. for 16 h. After cooling to ambient temperature, the mixture was diluted in ethyl acetate (75 mL) and the organic phase was washed with saturated ammonium chloride (2×25 mL) and brine (25 mL), dried over anhydrous magnesium sulfate, filtered, and concentrated in vacuo. Purification of the residue by preparative reverse phase HPLC, using a gradient of 10 to 85% acetonitrile in water, containing 0.5% formic acid as eluent, afforded the title compound as a colorless solid (0.012 g, 29% yield): ¹H NMR (400 MHz, DMSO-d₆) δ 10.13 (s, 1H), 8.90 (s, 2H), 8.19 (d, J=5.0 Hz, 1H), 7.40-7.35 (m, 1H), 7.32 (td, J=7.6, 1.6 Hz, 1H), 7.28-7.23 (m, 1H), 7.19 (td, J=7.5, 1.0 Hz, 1H), 6.81 (d, J=5.3 Hz, 1H), 4.22-4.11 (m, 4H), 3.16 (sept, J=6.9 Hz, 1H), 1.73 (t, J=8.8 Hz, 2H), 1.26 (d, J=6.9 Hz, 6H); ¹⁹F NMR (376 MHz, DMSO-d₆) 3-114.6, -137.7; (ES+) m/z 454.2 (M+1).

### Example 302

### Synthesis of N-(2-(1,1-difluoro-5-azaspiro[2.4]heptan-5-yl)-4-(2-fluorophenyl)pyridin-3-yl)-2-isopropylpyrimidine-5-carboxamide

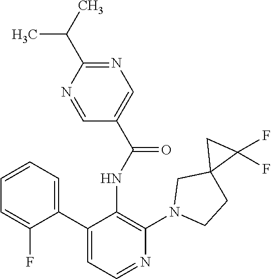 [SMILES: CC(C)c1ncc(C(=O)Nc2c(-c3ccccc3F)ccnc2N2CCC3(C2)CC3(F)F)cn1]

### Step 1. Preparation of 1,1-difluoro-5-(4-(2-fluorophenyl)-3-nitropyridin-2-yl)-5-azaspiro[2.4]heptane

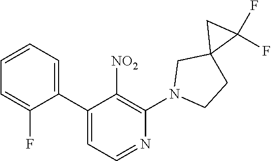 [SMILES: O=[N+]([O-])c1c(-c2ccccc2F)ccnc1N1CCC2(C1)CC2(F)F]

To a mixture of 2-chloro-4-(2-fluorophenyl)-3-nitro-pyridine (0.370 g, 1.46 mmol) in anhydrous N-methyl-2-pyrrolidone (7.3 mL) was added N,N-diisopropylethylamine (2.6 mL, 15 mmol) and 1,1-difluoro-5-azaspiro[2.4]heptane hydrochloride (0.250 g, 4.46 mmol). The reaction was stirred at 50° C. for 5 h. After cooling to ambient temperature, the mixture was diluted in ethyl acetate (100 mL) and the organic phase was washed with saturated ammonium chloride (30 mL), water (3×30 mL), and brine (30 mL), then dried over anhydrous magnesium sulfate, filtered, and concentrated in vacuo. The residue was purified by column chromatography, eluting with a gradient of 0 to 65% ethyl acetate in heptane, to afford the title compound as a yellow oil (0.392 g, 77% yield): MS (ES+) m/z 350.0 (M+1).

### Step 2. Preparation of 2-(1,1-difluoro-5-azaspiro[2.4]heptan-5-yl)-4-(2-fluorophenyl)pyridin-3-amine

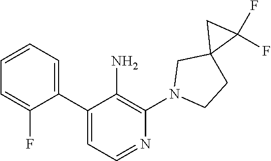 [SMILES: Nc1c(-c2ccccc2F)ccnc1N1CCC2(C1)CC2(F)F]

A mixture of 11,1-difluoro-5-(4-(2-fluorophenyl)-3-nitropyridin-2-yl)-5-azaspiro[2.4]heptane (0.392 g, 1.12 mmol) in methanol (5.6 mL) and ethyl acetate (5.6 mL) was degassed with nitrogen for 10 minutes. To the reaction mixture was added 10% palladium on carbon (0.075 g) and the reaction was stirred under hydrogen atmosphere and ambient temperature for 16 h. To the reaction mixture was added 10% palladium on carbon (0.075 g) and the reaction was stirred under hydrogen atmosphere and ambient temperature for 4 h. The mixture was diluted in 200 mL of ethyl acetate, filtered through a bed of diatomaceous earth (i.e., Celite®) and concentrated in vacuo. The residue was purified by column chromatography, eluting with a gradient of 0 to 70% ethyl acetate in heptane, to afford the title compound as a red oil (0.246 g, 67% yield): MS (ES+) m/z 320.0 (M+1).

### Step 3. Preparation of N-(2-(1,1-difluoro-5-azaspiro[2.4]heptan-5-yl)-4-(2-fluorophenyl)pyridin-3-yl)-2-isopropylpyrimidine-5-carboxamide

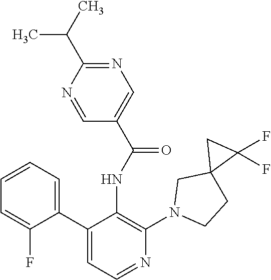 [SMILES: CC(C)c1ncc(C(=O)Nc2c(-c3ccccc3F)ccnc2N2CCC3(C2)CC3(F)F)cn1]

To a mixture of 2-(1,1-difluoro-5-azaspiro[2.4]heptan-5-yl)-4-(2-fluorophenyl)pyridin-3-amine (0.086 g, 0.27 mmol) in anhydrous tetrahydrofuran (5.4 mL) was added N,N-diisopropylethylamine (0.47 mL, 2.7 mmol), 2-chloro-1-methylpyridinium iodide (0.069 g, 0.27 mmol), and 2-isopropylpyrimidine-5-carboxylic acid (0.060 g, 0.36 mmol). The reaction mixture was stirred at 65° C. for 16 h. After cooling to ambient temperature, the mixture was diluted with ethyl acetate (100 mL) and the organic phase was washed with saturated ammonium chloride (2×35 mL) and brine (35 mL), dried over anhydrous magnesium sulfate, filtered, and concentrated in vacuo. The residue was purified by column chromatography, eluting with a gradient of 20 to 100% ethyl acetate in heptane, to afford the title compound as a colorless solid (0.089 g, 70% yield): ¹H NMR (400 MHz, DMSO-d₆) δ 10.16 (s, 1H), 8.85 (s, 2H), 8.17 (d, J=4.9 Hz, 1H), 7.37-7.32 (m, 1H), 7.31-7.28 (m, 1H), 7.26-7.21 (m, 1H), 7.17 (td, J=7.5, 0.8 Hz, 1H), 6.73 (d, J=4.9 Hz, 1H), 3.75-3.57 (m, 4H), 3.15 (sept, J=6.9 Hz, 1H), 2.12-1.97 (m, 2H), 1.66-1.54 (m, 2H), 1.26 (d, J=6.9 Hz, 6H); MS (ES+) m/z 488.2 (M+1).

### Biological Example 1

As disclosed above, typical assays for testing compounds of the disclosure are known, for example as disclosed in Crestey, F. et al., ACS Chem Neurosci (2015), Vol. 6, pp. 1302-1308, AA43279 (Frederiksen, K. et al., Eur J Neurosci (2017), Vol. 46, pp. 1887-1896) and Lu AE98134 (von Schoubyea, N. L. et a/., Neurosci Lett (2018), Vol. 662, pp. 29-35) which employs the use of automated planar patch clamp techniques to study the effects of the chemical agent on the gating of sodium channels. The sodium channel isoforms of interest are stably expressed in Human Embryonic Kidney Cells and the currents that flow through those channels in response to a depolarizing voltage clamp step from −120 mV to 0 mV are measured in the presence of increasing concentrations of the chemical agents. The area under the sodium current trace which correlates to the magnitude of sodium flux through the cell mebrane is used to quantify the effects on gating of the channels. Other parameters that are measured in the assay include the peak current, time constant of open state inactivation and the voltage dependence of steady state inactivation properties. The concentration responses are used to determine potency of each chemical agents effects on modulating the sodium channel isoform gating.

Each of the aforementioned references are hereby incorporated by reference in their entirety.

TABLE 2
Biological activity of representative
compounds of formula (I) or (II)

| Example No. | EC₅₀ | EmpiricalEₘₐₓ |
|---|---|---|
| 1 | +++ | +++ |
| 2 | +++ | ++ |
| 3 | ++++ | +++ |
| 4 | +++ | ++++ |
| 5 | ++++ | +++ |
| 6 | + | ++ |
| 7 | + | ++ |
| 8 | ++ | ++ |
| 9 | + | ++ |
| 10 | +++ | +++ |
| 11 | ++ | ++ |
| 12 | ++++ | + |
| 13 | + | + |
| 14 | + | + |
| 15 | + | + |
| 16 | ++++ | + |
| 17 | ++ | + |
| 18 | ++ | ++ |
| 19 | +++ | +++ |
| 20 | ++++ | ++++ |
| 21 | ++++ | +++ |
| 22 | +++ | ++ |
| 23 | +++ | +++ |
| 24 | ++++ | +++ |
| 25 | ++++ | ++++ |
| 26 | ++++ | ++++ |
| 27 | ++++ | ++++ |
| 28 | ++++ | ++++ |
| 29 | ++++ | + |
| 30 | ++++ | + |
| 31 | ++++ | ++++ |
| 32 | +++ | ++++ |
| 33 | +++ | +++ |
| 34 | ++++ | ++++ |
| 35 | + | + |
| 36 | ++ | ++ |
| 37 | +++ | +++ |
| 38 | ++++ | ++++ |
| 39 | ++++ | ++++ |
| 40 | ++++ | ++++ |
| 41 | ++++ | ++++ |
| 42 | +++ | ++++ |
| 43 | ++++ | ++++ |
| 44 | +++ | +++ |
| 45 | ++++ | +++ |
| 46 | +++ | ++ |
| 47 | +++ | +++ |
| 48 | +++ | +++ |
| 49 | ++++ | ++++ |
| 50 | ++++ | ++++ |
| 51 | ++++ | ++++ |
| 52 | + | + |
| 53 | + | ++ |
| 54 | ++++ | +++ |
| 55 | + | ++ |
| 56 | ++++ | +++ |
| 57 | ++++ | +++ |
| 58 | ++++ | +++ |
| 59 | +++ | ++ |
| 60 | ++++ | +++ |
| 61 | + | ++ |
| 62 | ++ | ++ |
| 63 | +++ | ++++ |
| 64 | +++ | ++++ |
| 65 | +++ | +++ |
| 66 | ++++ | ++ |
| 67 | +++ | +++ |
| 68 | +++ | +++ |
| 69 | ++++ | +++ |
| 70 | ++++ | +++ |
| 71 | +++ | +++ |
| 72 | ++++ | ++++ |
| 73 | +++ | ++ |
| 74 | +++ | ++++ |
| 75 | ++++ | ++++ |
| 76 | ++++ | ++++ |
| 77 | ++++ | +++ |
| 78 | ++++ | ++++ |
| 79 | ++++ | ++++ |
| 80 | ++++ | +++ |
| 81 | +++ | +++ |
| 82 | ++++ | ++++ |
| 83 | +++ | ++++ |
| 84 | +++ | +++ |
| 85 | +++ | ++++ |
| 86 | ++++ | ++++ |
| 87 | +++ | +++ |
| 88 | ++++ | ++++ |
| 89 | + | + |
| 90 | + | ++ |
| 91 | +++ | ++++ |
| 92 | ++++ | +++ |
| 93 | ++++ | ++++ |
| 94 | ++++ | +++ |
| 95 | +++ | +++ |
| 96 | ++++ | ++++ |
| 97 | +++ | +++ |
| 98 | ++++ | ++++ |
| 99 | +++ | ++ |
| 100 | ++++ | ++++ |
| 101 | ++++ | ++++ |
| 102 | +++ | ++++ |
| 103 | ++++ | ++++ |
| 104 | +++ | +++ |
| 105 | ++++ | +++ |
| 106 | +++ | ++ |
| 107 | +++ | +++ |
| 108 | ++++ | + |
| 109 | ++++ | +++ |
| 110 | ++ | ++ |
| 111 | ++++ | ++++ |
| 112 | ++++ | ++++ |
| 113 | ++++ | ++ |
| 114 | ++++ | +++ |
| 115 | ++ | ++ |
| 116 | ++++ | +++ |
| 117 | +++ | ++ |
| 118 | +++ | ++ |
| 119 | ++++ | +++ |
| 120 | ++++ | +++ |
| 121 | ++++ | ++++ |
| 122 | ++++ | ++++ |
| 123 | ++++ | +++ |
| 124 | ++++ | +++ |
| 125 | ++++ | +++ |
| 126 | ++++ | +++ |
| 127 | +++ | +++ |
| 128 | +++ | ++++ |
| 129 | ++++ | +++ |
| 130 | +++ | +++ |
| 131 | +++ | +++ |
| 132 | ++++ | ++++ |
| 133 | ++++ | ++++ |
| 134 | ++++ | ++++ |
| 135 | ++++ | ++++ |
| 136 | + | ++ |
| 137 | ++++ | ++++ |
| 138 | ++++ | ++++ |
| 139 | ++++ | +++ |
| 140 | ++++ | ++++ |
| 141 | +++ | +++ |
| 142 | ++++ | ++++ |
| 143 | +++ | +++ |
| 144 | +++ | +++ |
| 145 | ++++ | ++++ |
| 146 | ++++ | +++ |
| 147 | +++ | +++ |
| 148 | +++ | ++ |
| 149 | ++++ | ++++ |
| 150 | ++++ | ++++ |
| 151 | ++++ | ++++ |
| 152 | ++++ | ++++ |
| 153 | + | ++ |
| 154 | ++++ | ++++ |
| 155 | ++++ | +++ |
| 156 | + | + |
| 157 | + | + |
| 158 | ++++ | ++++ |
| 159 | +++ | ++ |
| 160 | ++++ | ++++ |
| 161 | +++ | +++ |
| 162 | ++++ | ++++ |
| 163 | +++ | +++ |
| 164 | +++ | ++++ |
| 165 | ++++ | ++++ |
| 166 | ++++ | +++ |
| 167 | ++++ | ++++ |
| 168 | ++ | + |
| 169 | ++++ | + |
| 170 | ++++ | +++ |
| 171 | ++++ | +++ |
| 172 | ++++ | ++++ |
| 173 | ++++ | ++++ |
| 174 | ++++ | ++++ |
| 175 | ++++ | ++++ |
| 176 | ++++ | ++++ |
| 177 | ++++ | ++++ |
| 178 | ++++ | +++ |
| 179 | ++++ | ++++ |
| 180 | +++ | ++ |
| 181 | +++ | ++++ |
| 182 | ++++ | ++++ |
| 183 | +++ | ++++ |
| 184 | ++++ | ++++ |
| 185 | ++++ | ++++ |
| 186 | ++++ | +++ |
| 187 | +++ | +++ |
| 188 | ++++ | ++++ |
| 189 | ++++ | ++++ |
| 190 | ++++ | ++++ |
| 191 | +++ | — |
| 192 | ++++ | — |
| 193 | ++++ | — |
| 194 | ++ | — |
| 195 | ++++ | — |
| 196 | ++ | — |
| 197 | ++ | — |
| 198 | ++ | — |
| 199 | ++ | — |
| 200 | +++ | — |
| 201 | ++++ | — |
| 202 | ++++ | — |
| 203 | +++ | — |
| 204 | ++++ | — |
| 205 | +++ | — |
| 206 | ++ | — |
| 207 | ++++ | — |
| 208 | ++ | — |
| 209 | ++++ | — |
| 210 | +++ | — |
| 211 | +++ | — |
| 212 | +++ | — |
| 213 | ++++ | — |
| 214 | ++++ | — |
| 215 | +++ | — |
| 216 | ++++ | — |
| 217 | ++ | — |
| 218 | +++ | — |
| 219 | ++ | — |
| 220 | +++ | — |
| 221 | ++ | — |
| 222 | +++ | — |
| 223 | ++ | — |
| 224 | ++++ | — |
| 225 | ++++ | — |
| 226 | ++++ | — |
| 227 | ++++ | — |
| 228 | +++ | — |
| 229 | +++ | — |
| 230 | +++ | — |
| 231 | ++++ | — |
| 232 | ++ | — |
| 233 | +++ | — |
| 234 | ++ | — |
| 235 | ++++ | — |
| 236 | +++ | — |
| 237 | ++++ | — |
| 238 | ++++ | — |
| 239 | ++++ | — |
| 240 | +++ | — |
| 241 | ++++ | — |
| 242 | +++ | — |
| 243 | +++ | — |
| 244 | ++++ | — |
| 245 | +++ | — |
| 246 | ++++ | — |
| 247 | ++++ | — |
| 248 | ++++ | — |
| 249 | +++ | — |
| 250 | +++ | — |
| 251 | +++ | — |
| 252 | +++ | — |
| 253 | ++++ | — |
| 254 | +++ | — |
| 255 | +++ | — |
| 256 | ++ | — |
| 257 | ++++ | — |
| 258 | ++++ | — |
| 259 | ++++ | — |
| 260 | ++++ | — |
| 261 | ++++ | — |
| 262 | — | — |
| 263 | — | — |
| 264 | ++ | — |
| 265 | ++ | — |
| 266 | ++ | — |
| 267 | ++++ | — |
| 268 | ++ | — |
| 269 | ++++ | — |
| 270 | ++ | — |
| 271 | +++ | — |
| 272 | +++ | — |
| 273 | +++ | — |
| 274 | ++ | — |
| 275 | +++ | — |
| 276 | ++++ | — |
| 277 | +++ | — |
| 278 | ++ | — |
| 279 | ++ | — |
| 280 | ++ | — |
| 281 | ++ | — |
| 282 | ++ | — |
| 283 | ++ | — |
| 284 | +++ | — |
| 285 | +++ | — |
| 286 | ++ | — |
| 287 | +++ | — |
| 288 | ++++ | — |
| 289 | +++ | — |
| 290 | ++ | — |
| 291 | ++ | — |
| 292 | ++ | — |
| 293 | ++ | — |
| 294 | ++ | — |
| 295 | ++ | — |
| 296 | ++++ | — |
| 297 | ++++ | — |
| 298 | +++ | — |
| 299 | ++++ | — |
| 300 | ++++ | — |
| 301 | ++++ | — |
| 302 | +++ | — |
| 303 | ++++ | — |
| 304 | ++++ | — |
| 305 | ++++ | — |
| 306 | ++++ | — |
| 307 | ++++ | — |

For EC₅₀ values:
++++ indicates a value less than 1 μM
+++ indicates a value from 1 up to 10 μM
++ indicates a value from 10 up to 50 μM
+ indicates a value of 50 μM or more
For Empirical Eₘₐₓ values:
++++ indicates a value greater than 7.5
+++ indicates a value from 5.0 up to 7.5 μM
++ indicates a value from 2.0 up to 5.0 μM
+ indicates a value less than 2.0 μM

All of the U.S. patents, U.S. patent application publications, U.S. patent applications, foreign patents, foreign patent applications and non-patent publications referred to in this specification are incorporated herein by reference in their entireties.

Although the foregoing disclosure has been described in some detail to facilitate understanding, it will be apparent that certain changes and modifications may be practiced within the scope of the appended claims. Accordingly, the described embodiments are to be considered as illustrative and not restrictive, and the disclosure is not to be limited to the details given herein, but may be modified within the scope and equivalents of the appended claims.

1. A compound of formula (I): 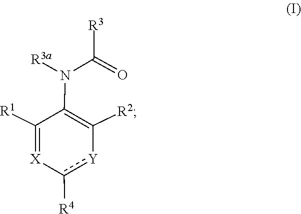
wherein: [symbol]
represents a double or single bond such that all valences are satisfied;
Y is N or NR^{4a};
X is C(R⁷) or N;
R¹ is selected from: 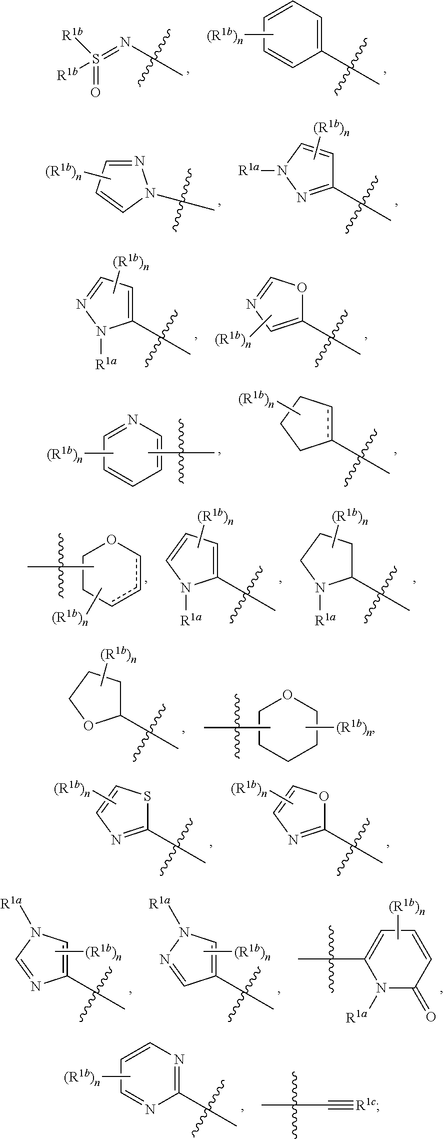
wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
R^{1c} is N or —Si(CH₃)₃;
R² is selected from: 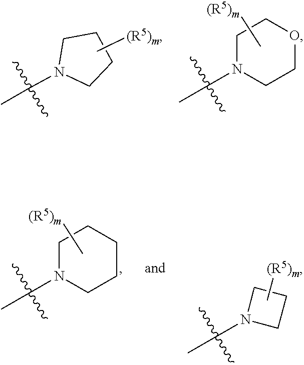
wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain;
R³ is alkyl, —R⁸—N(R⁹)₂, —R⁸—OR⁹, or
R³ is selected from: 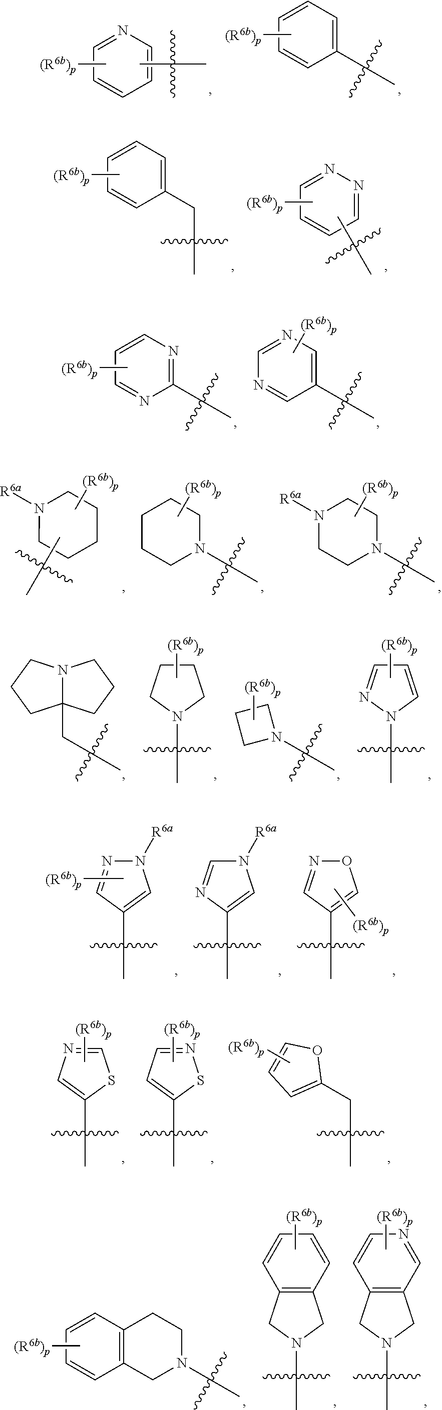 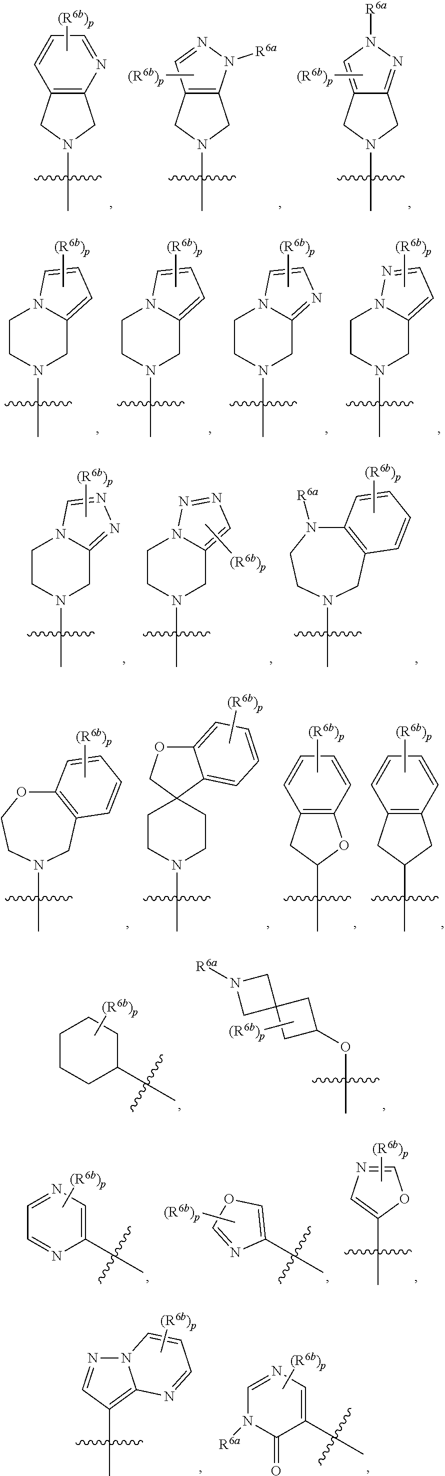
wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain;
or an occurrence of R^{6b} and an occurrence of Rib join to form an optionally substituted alkylene chain;
R^{3a} is hydrogen or alkyl;
R⁴ is hydrogen, alkyl, —R⁸—OR⁹, halo, haloalkyl, or cyano;
or R⁴ together with the carbon to which it is attached, joins with R^{4a} together with the nitrogen to which it is attached to form an optionally substituted 5-membered N-heteroaryl;
R⁷ is hydrogen, alkyl, halo, or —R⁸—OR⁹
each R⁸ is independently a direct bond or an optionally substituted alkylene chain;
each R⁹ is independently hydrogen, alkyl, haloalkyl, carboxyalkyl, optionally substituted cycloalkyl,
optionally substituted cycloalkylalkyl, or optionally substituted aryl; and
or two R⁹'s, together with the nitrogen to which they are both attached, form an optionally substituted heterocyclyl;
provided that:
when X is N, R³ is selected from: 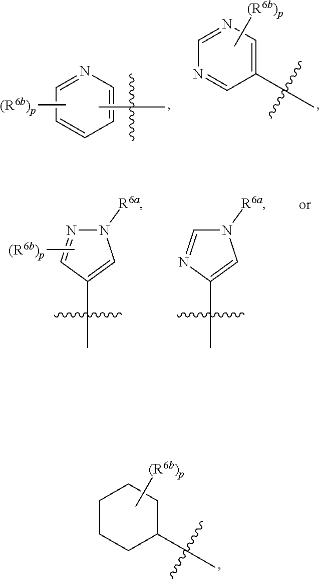
as a stereoisomer, enantiomer, or tautomer thereof or a mixture thereof, or a pharmaceutically acceptable salt, solvate, or prodrug thereof.

2. The compound of claim 1, wherein the compound has the following formula (Ia): 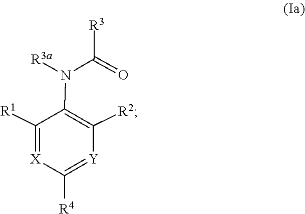
X, Rᵢ, R², R³, R^{3a}, and R⁴ are each as defined above in claim 1;
as a stereoisomer, enantiomer, or tautomer thereof or a mixture thereof, or a pharmaceutically acceptable salt, solvate, or prodrug thereof.

3. The compound of claim 1, wherein the compound has the following formula (Ib): 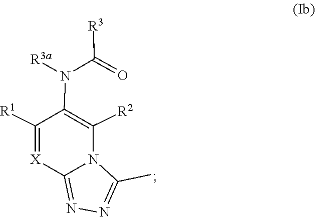
X, Rᵢ, R², R³, R^{3a}, and R⁴ are each as defined above in claim 1;
as a stereoisomer, enantiomer, or tautomer thereof or a mixture thereof; or a pharmaceutically acceptable salt, solvate, or prodrug thereof.

4-8. (canceled)

9. The compound of claim 1, wherein:
R¹ is selected from: 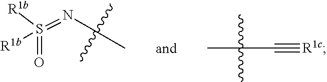
wherein:
each Rib is independently halo, alkyl, haloalkyl, cyano, heterocyclylalkyl,
—R⁸—N(R⁹)₂, —R⁸—C(═O)N(R⁹)₂, or —R⁸—OR⁹
R^{1c} is N or —Si(CH₃)₃;
as a stereoisomer, enantiomer, or tautomer thereof or a mixture thereof, or a pharmaceutically acceptable salt, solvate, or prodrug thereof.

10. The compound of claim 1, wherein:
R¹ is selected from: 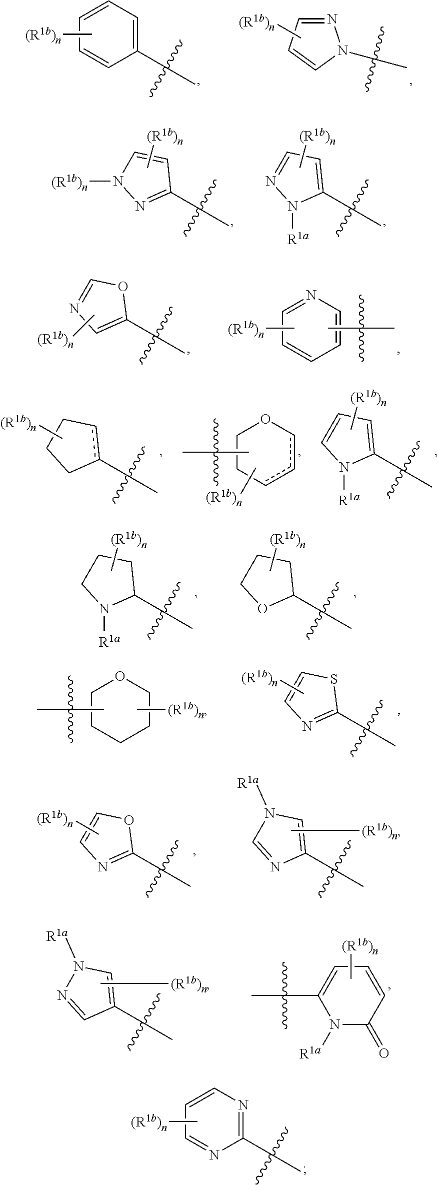
wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl, —R⁸—N(R⁹)₂, —R⁸—C(═O)N(R⁹)₂, or —R⁸—OR⁹
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
as a stereoisomer, enantiomer, or tautomer thereof or a mixture thereof, or a pharmaceutically acceptable salt, solvate, or prodrug thereof.

11. The compound of f claim 1, wherein:
Rᵢ is selected from: 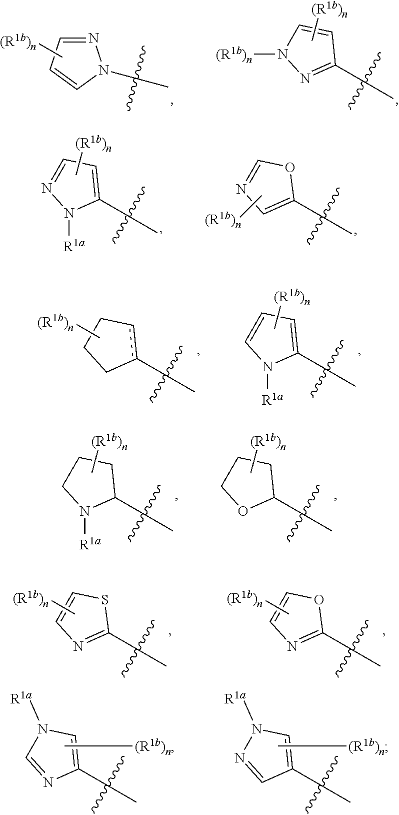
wherein:
each occurrence of [symbol] independently represents a double or single bond such that all valences are satisfied;
n is 0, 1, 2, 3, 4, or 5;
R^{1a} is hydrogen, or alkyl;
each Rib is independently halo, alkyl, haloalkyl, cyano, heterocyclylalkyl,
—R⁸—N(R⁹)₂, —R⁸—C(═O)N(R⁹)₂, or —R⁸—OR⁹
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
as a stereoisomer, enantiomer, or tautomer thereof or a mixture thereof, or a pharmaceutically acceptable salt, solvate, or prodrug thereof.

12. (canceled)

13. The compound of claim 1, wherein:
Rᵢ is selected from: 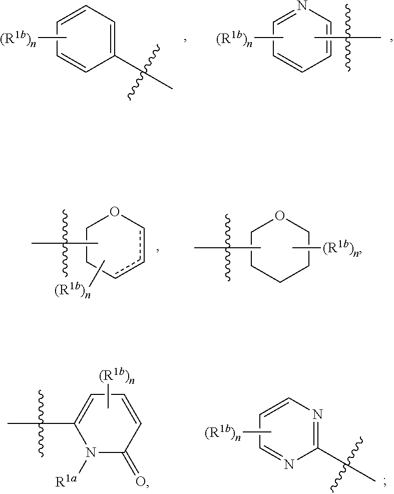
wherein:
each occurrence of [symbol] independently represents a double or single bond such that all valences are satisfied;
n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
as a stereoisomer, enantiomer, or tautomer thereof or a mixture thereof, or a pharmaceutically acceptable salt, solvate, or prodrug thereof.

14-15. (canceled)

16. The compound of claim 1, wherein
R¹ has one of the following structures: 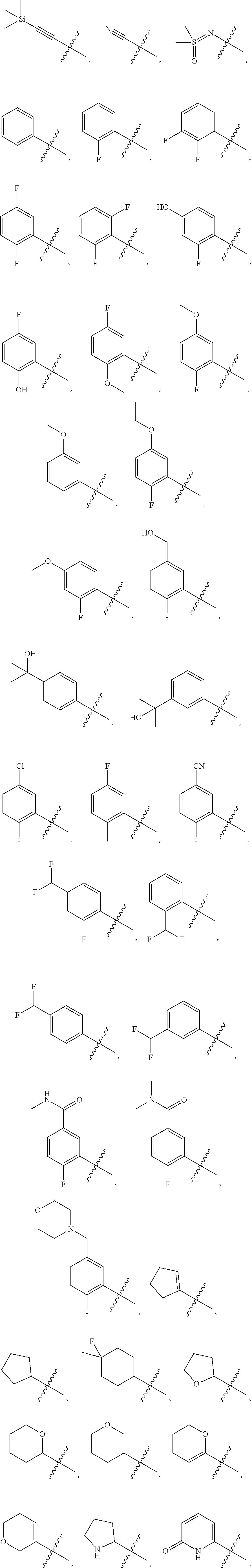 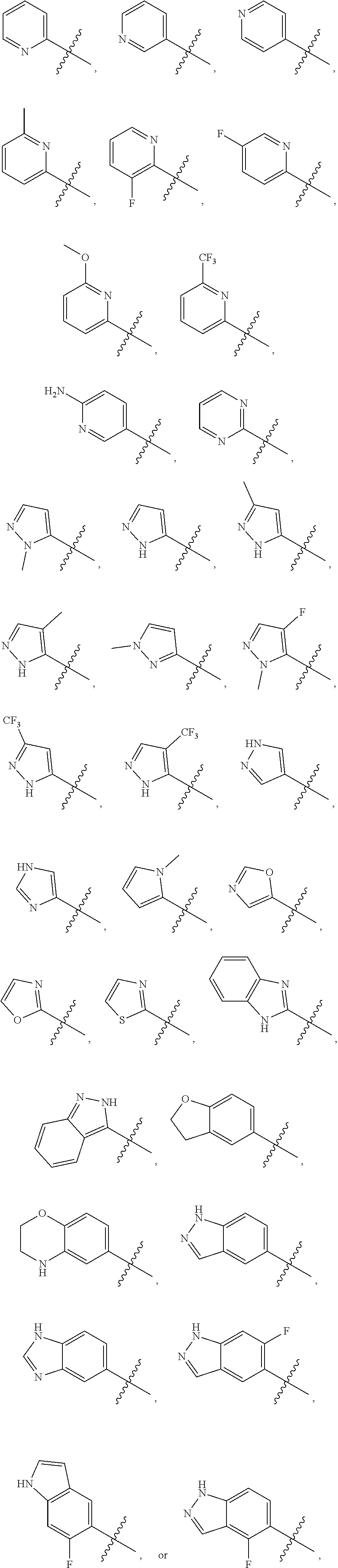
as a stereoisomer, enantiomer, or tautomer thereof or a mixture thereof, or a pharmaceutically acceptable salt, solvate, or prodrug thereof.

17-26. (canceled)

27. The compound of claim 1, wherein:
R² has one of the following structures: 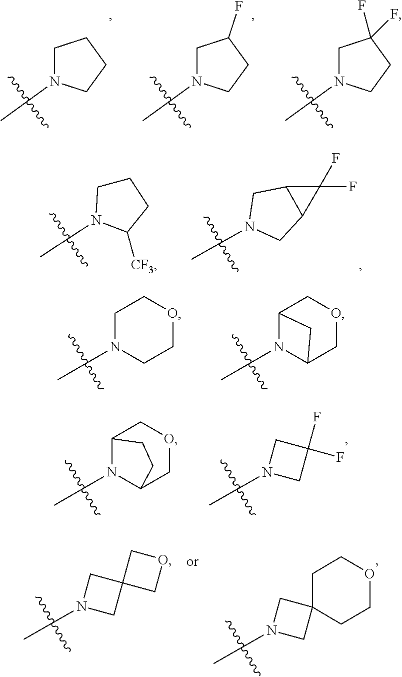
as a stereoisomer, enantiomer, or tautomer thereof or a mixture thereof, or a pharmaceutically acceptable salt, solvate, or prodrug thereof.

28. The compound of claim 1, wherein:
R² has one of the following structures: 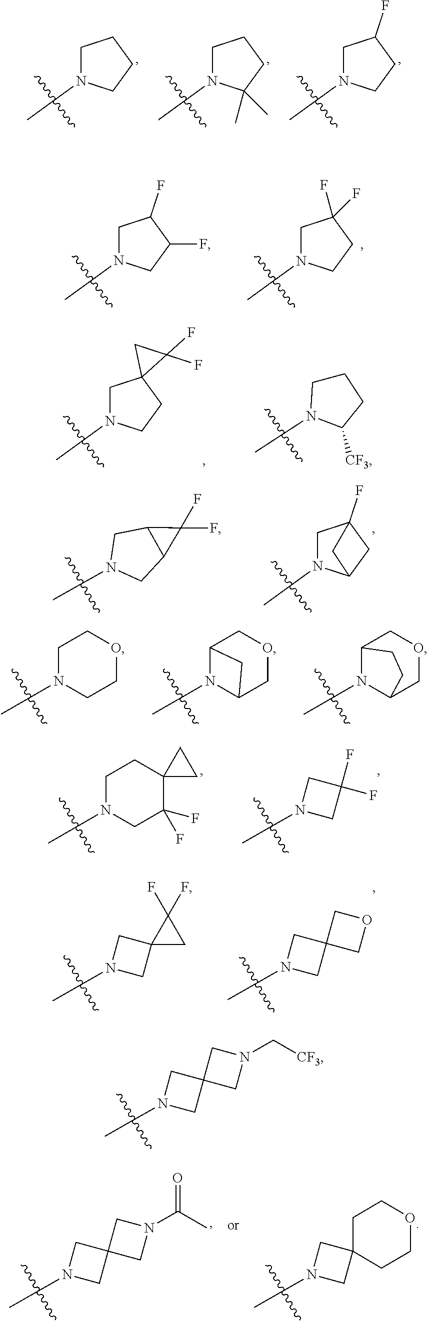
as a stereoisomer, enantiomer, or tautomer thereof or a mixture thereof, or a pharmaceutically acceptable salt, solvate, or prodrug thereof.

29-38. (canceled)

39. The compound of claim 1, wherein:
R³ has one of the following structures: 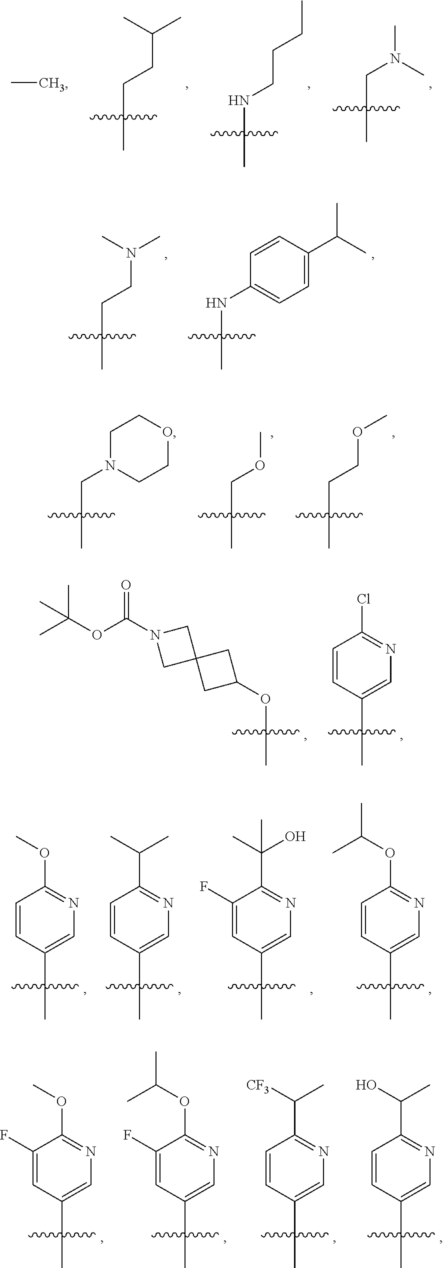 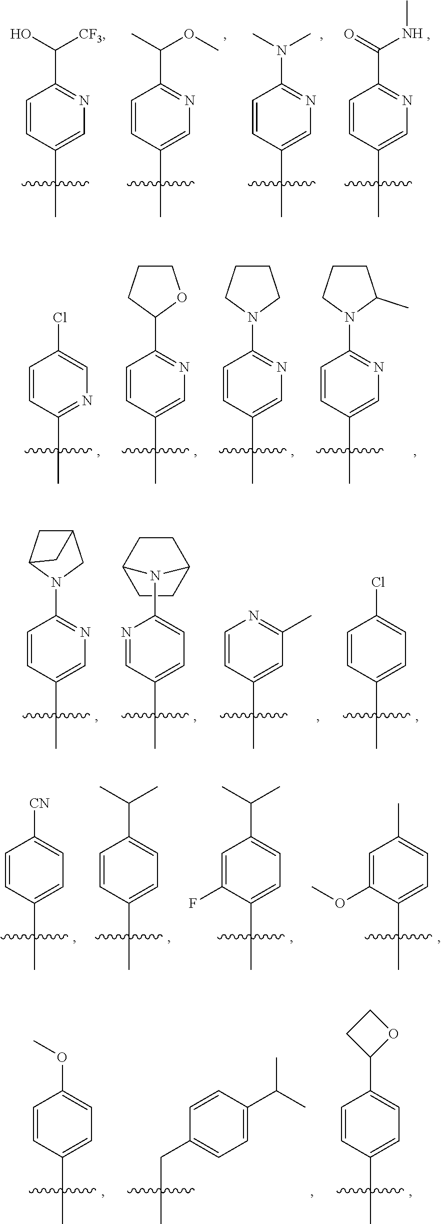 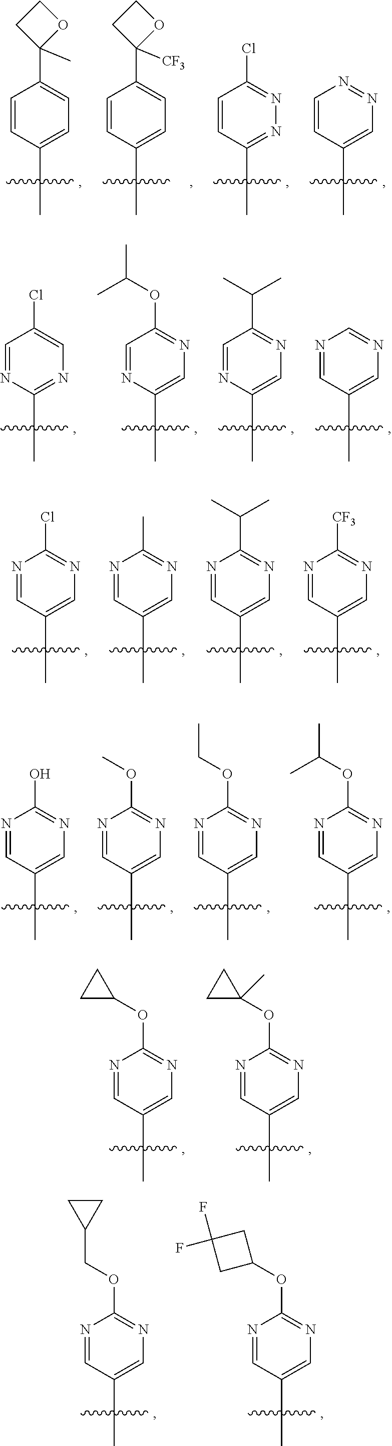 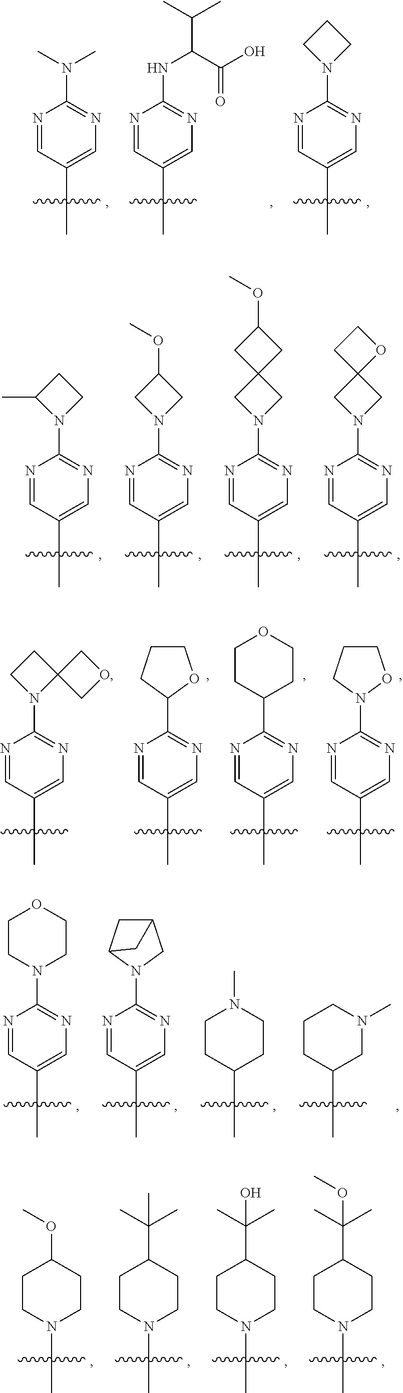 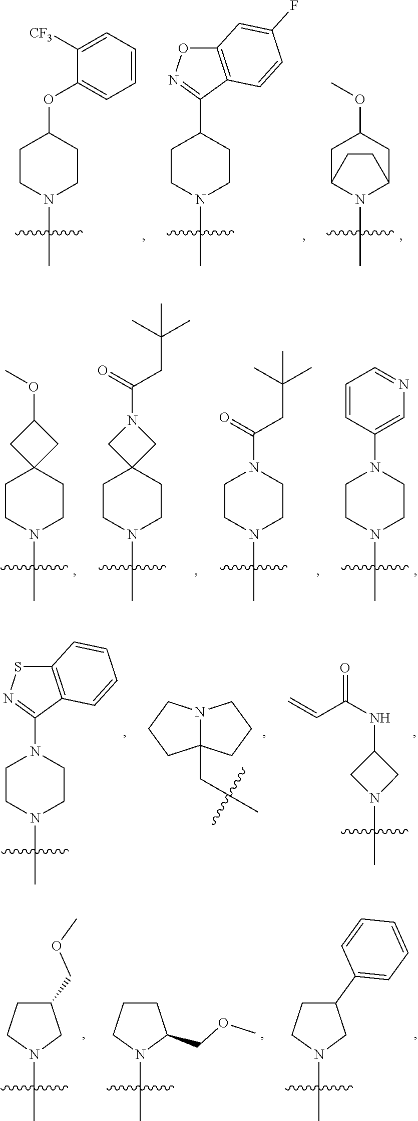 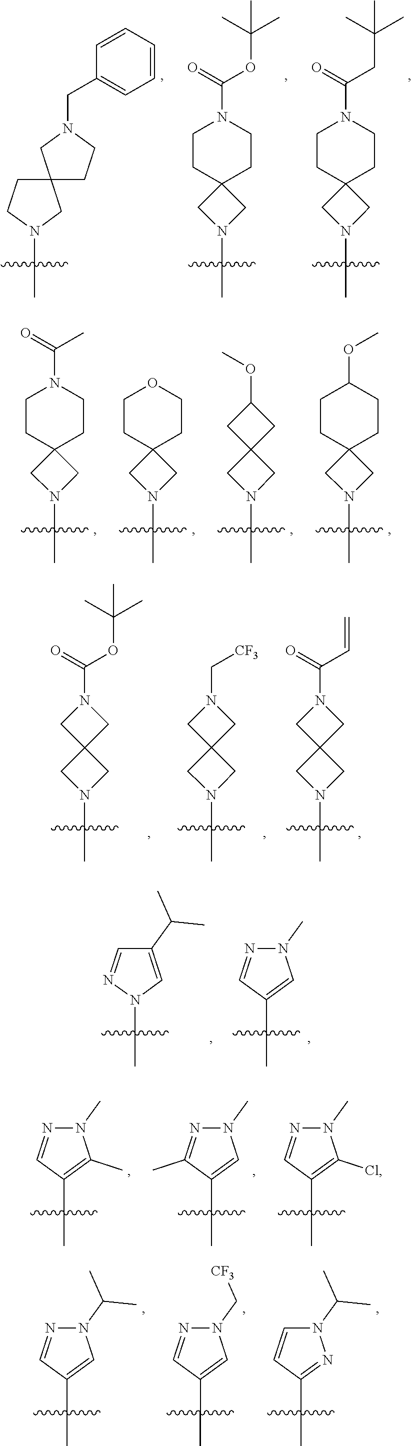 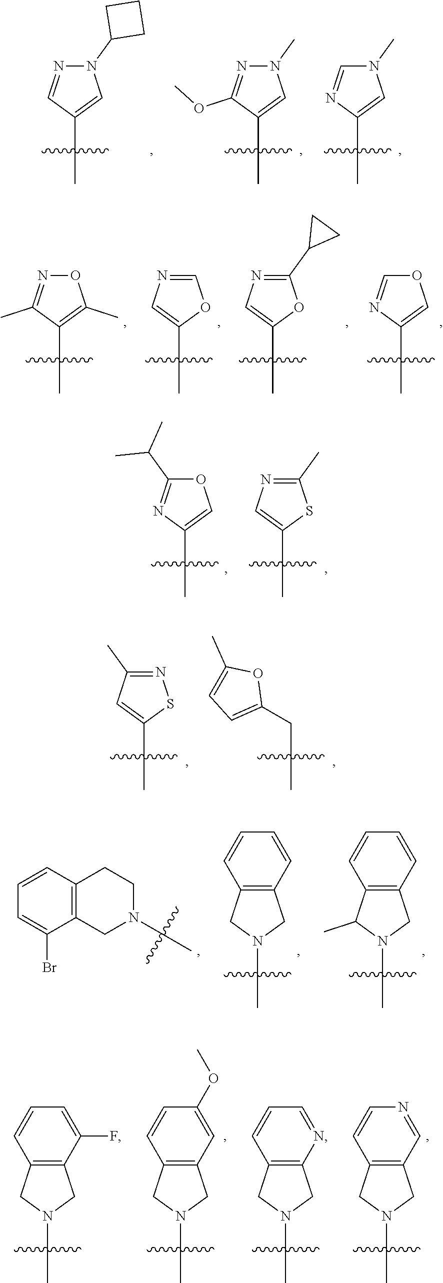 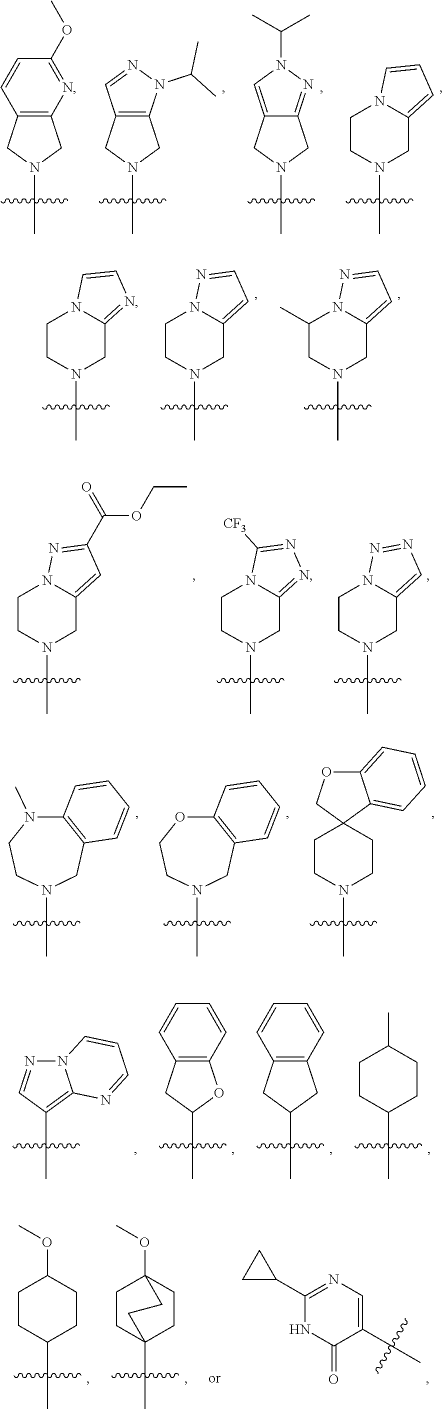
as a stereoisomer, enantiomer, or tautomer thereof or a mixture thereof, or a pharmaceutically acceptable salt, solvate, or prodrug thereof.

40-56. (canceled)

57. The compound of claim 1, wherein:
R³ and R¹ together have one of the following structures: 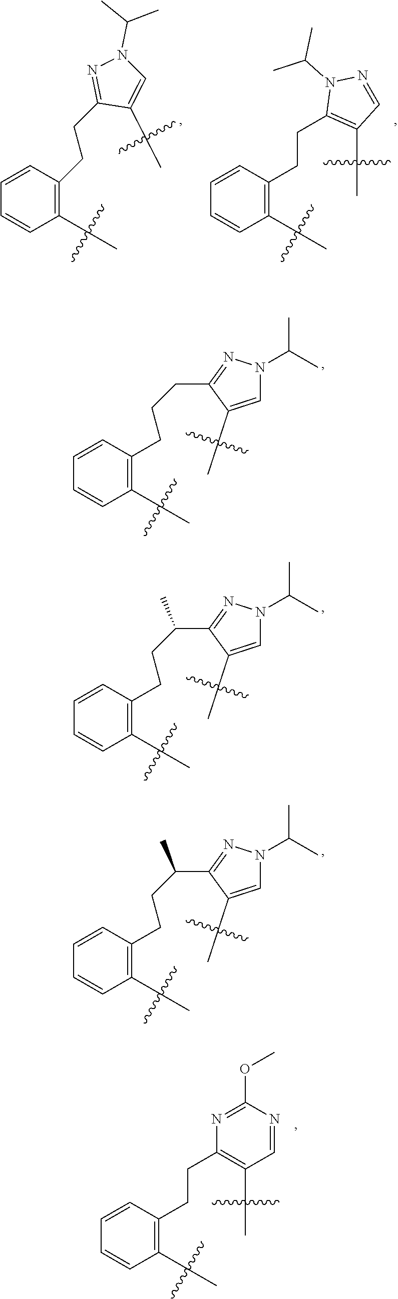 [SMILES: *c1ccccc1CCc1nn(C(C)C)cc1* ; *c1ccccc1CCc1c(*)cnn1C(C)C ; *c1ccccc1CCCc1nn(C(C)C)cc1* ; *c1ccccc1CC[C@H](C)c1nn(C(C)C)cc1* ; *c1ccccc1CC[C@@H](C)c1nn(C(C)C)cc1* ; *c1ccccc1CCc1nc(OC)ncc1*]
as a stereoisomer, enantiomer, or tautomer thereof or a mixture thereof, or a pharmaceutically acceptable salt, solvate, or prodrug thereof.

58. The compound of claim 1, wherein:
R^{3a} is hydrogen;
as a stereoisomer, enantiomer, or tautomer thereof or a mixture thereof, or a pharmaceutically acceptable salt, solvate, or prodrug thereof.

59. (canceled)

60. The compound of claim 1, wherein:
R^{3a} is methyl;
as a stereoisomer, enantiomer, or tautomer thereof or a mixture thereof, or a pharmaceutically acceptable salt, solvate, or prodrug thereof.

61. The compound of claim 1, wherein:
R⁴ is hydrogen, methyl, fluoro, chloro, —OH, —OCH₃, —CF₃, or cyano;
as a stereoisomer, enantiomer, or tautomer thereof or a mixture thereof, or a pharmaceutically acceptable salt, solvate, or prodrug thereof.

62-71. (canceled)

72. The compound of nm claim 1, wherein:
R⁷ is methyl;
as a stereoisomer, enantiomer, or tautomer thereof or a mixture thereof, or a pharmaceutically acceptable salt, solvate, or prodrug thereof.

73. The compound of claim 1, wherein the compound is selected from the group consisting of: 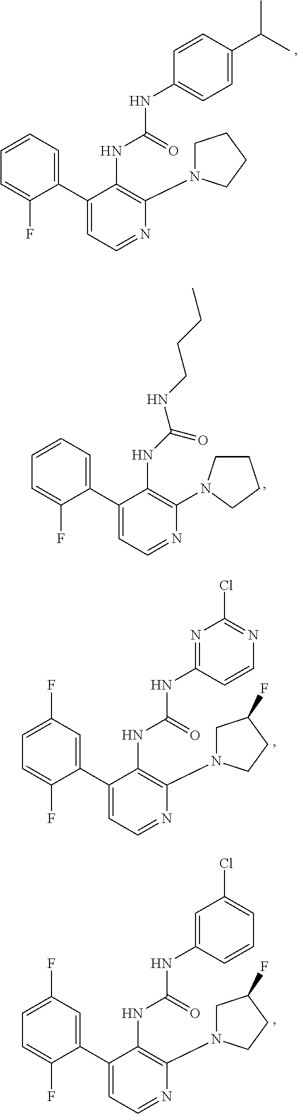 [SMILES: CC(C)c1ccc(NC(=O)Nc2c(-c3ccccc3F)ccnc2N2CCCC2)cc1 ; CCCCNC(=O)Nc1c(-c2ccccc2F)ccnc1N1CCCC1 ; O=C(Nc1ccnc(Cl)n1)Nc1c(-c2cc(F)ccc2F)ccnc1N1CC[C@H](F)C1 ; O=C(Nc1cccc(Cl)c1)Nc1c(-c2cc(F)ccc2F)ccnc1N1CC[C@H](F)C1] 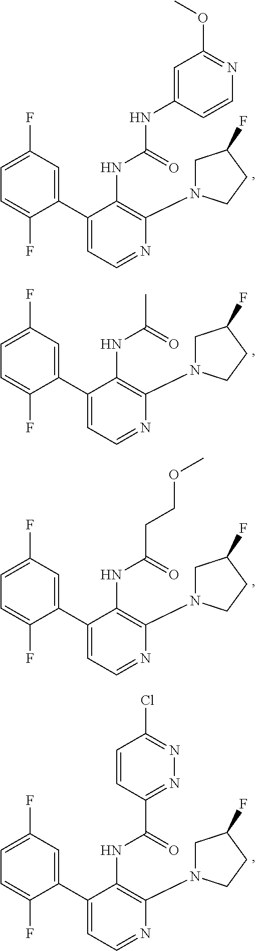 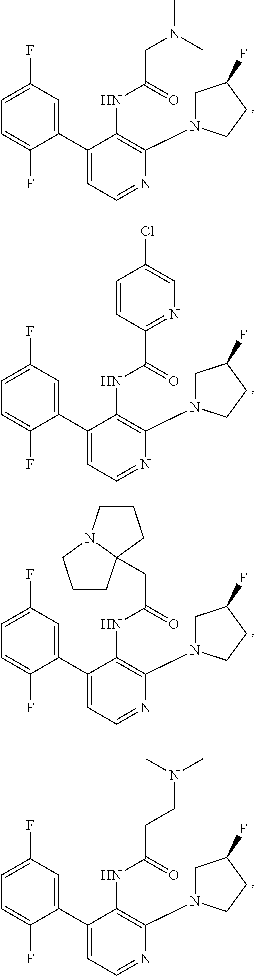 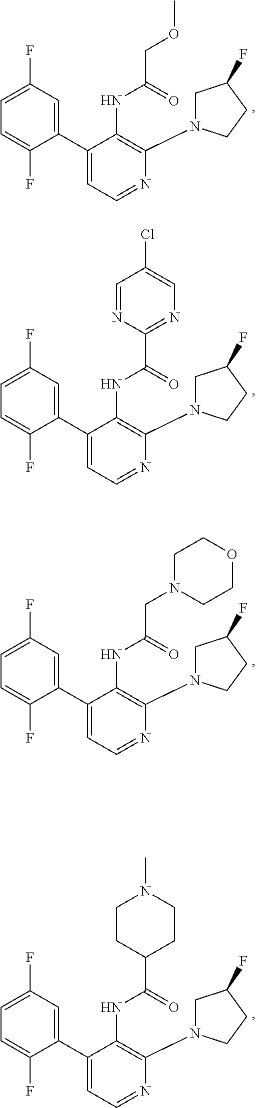 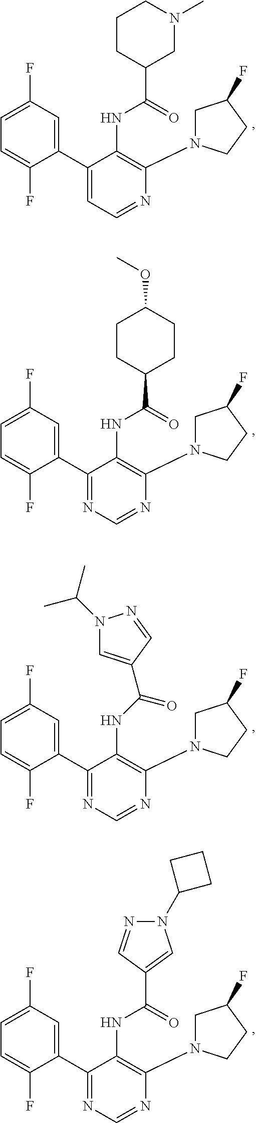 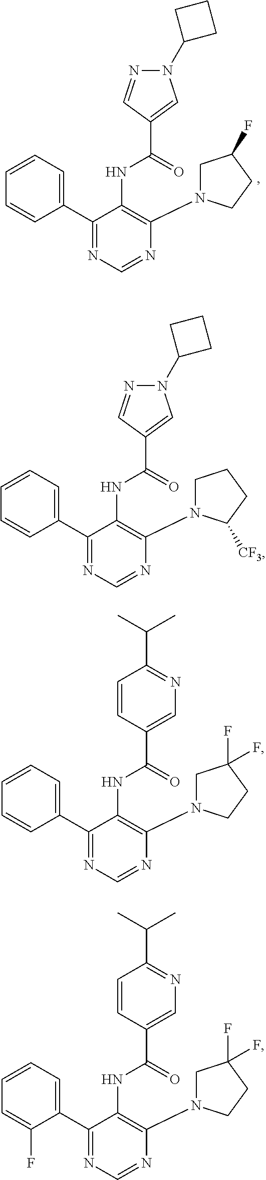 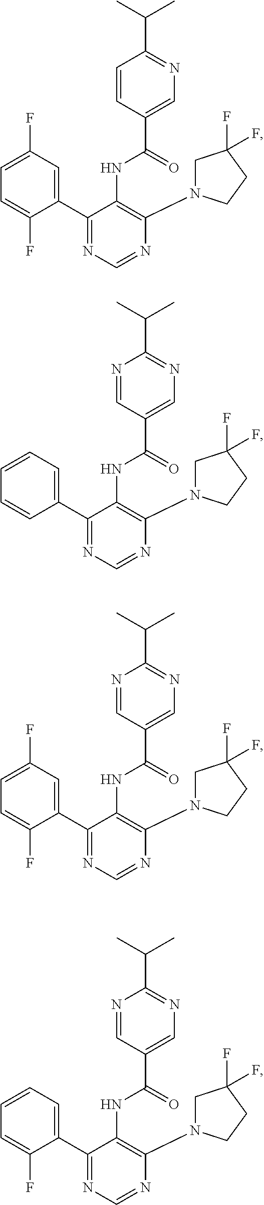 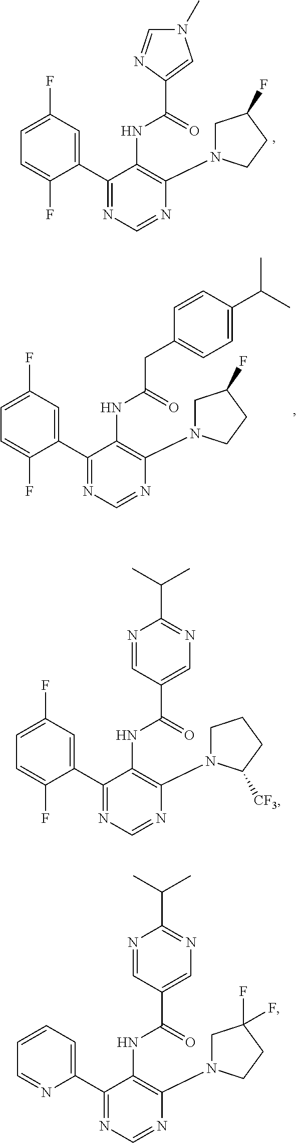 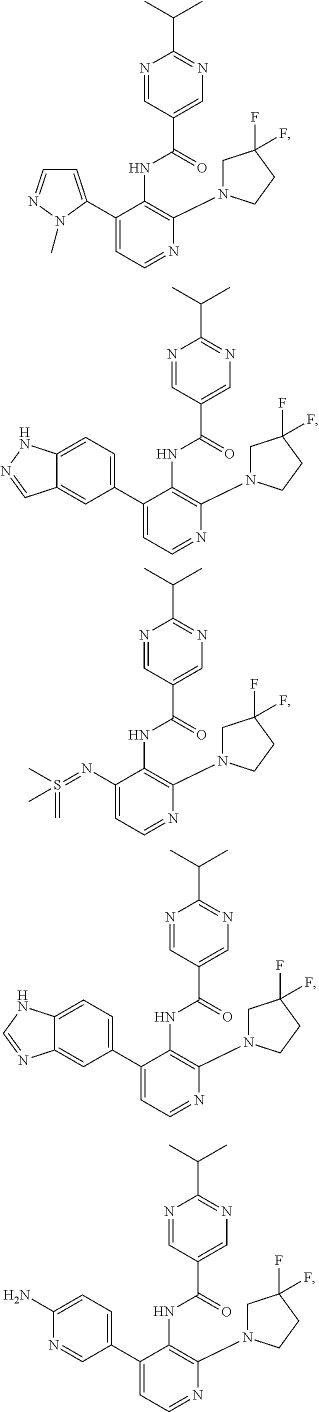 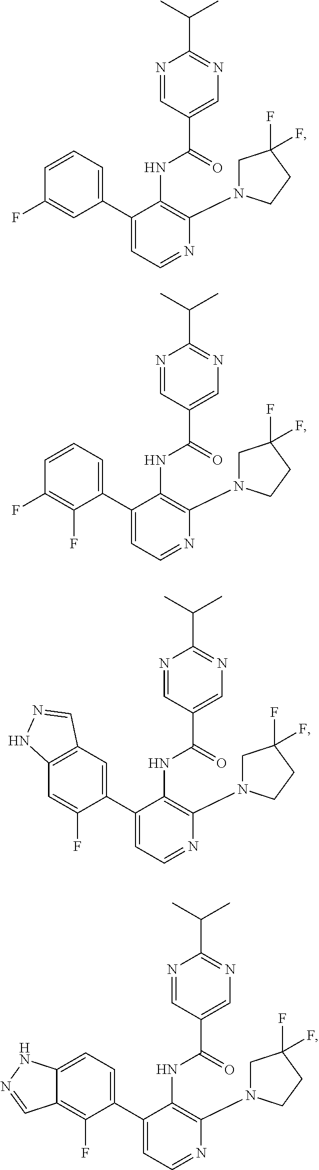 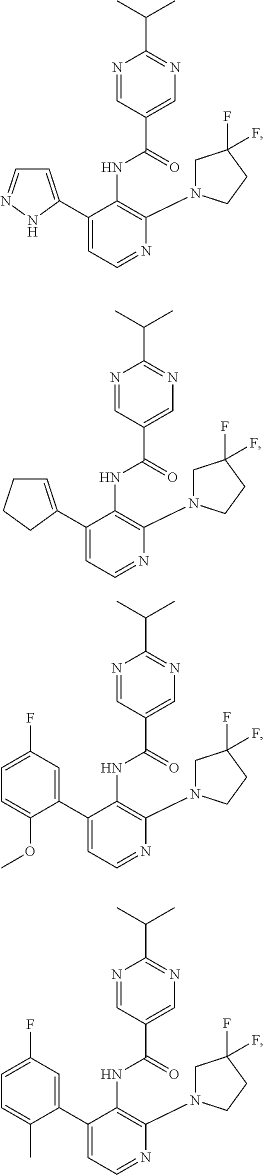 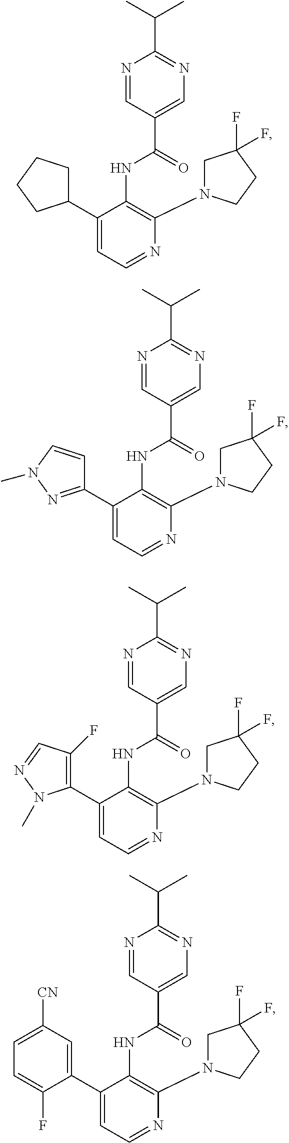 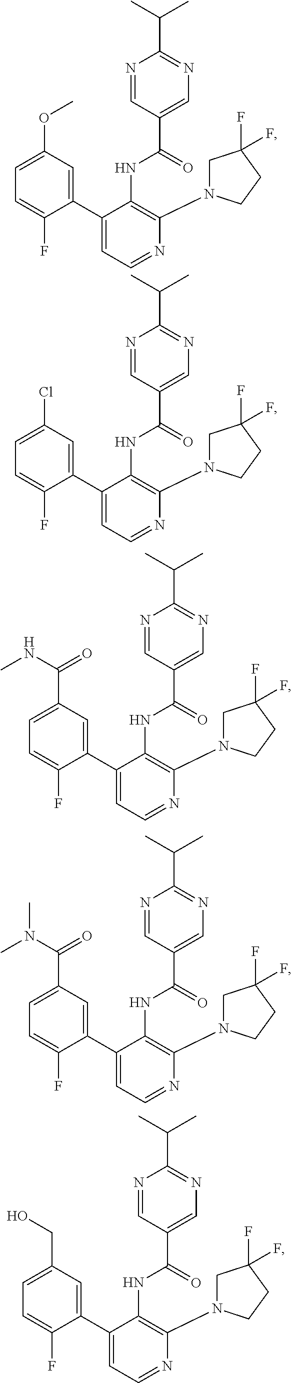 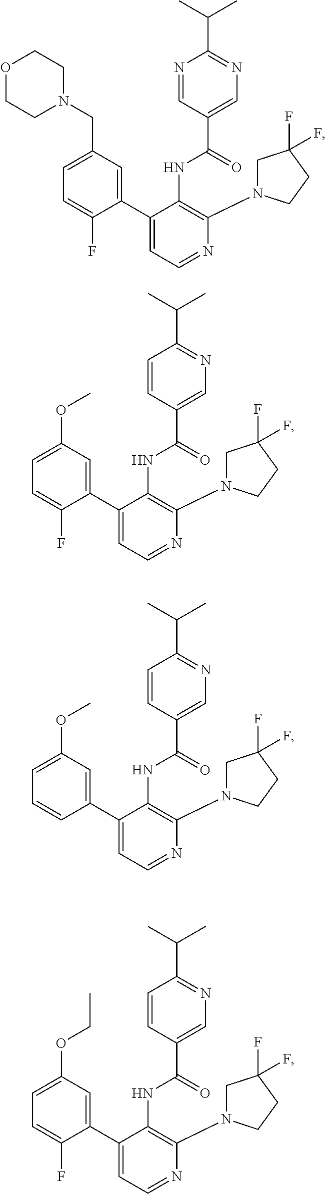 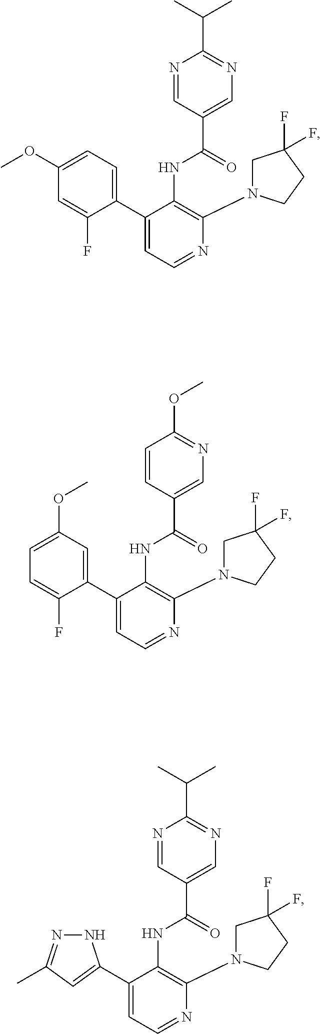 [SMILES: COc1ccc(-c2ccnc(N3CCC(F)(F)C3)c2NC(=O)c2cnc(C(C)C)nc2)c(F)c1 ; COc1ccc(F)c(-c2ccnc(N3CCC(F)(F)C3)c2NC(=O)c2ccc(OC)nc2)c1 ; Cc1cc(-c2ccnc(N3CCC(F)(F)C3)c2NC(=O)c2cnc(C(C)C)nc2)[nH]n1] 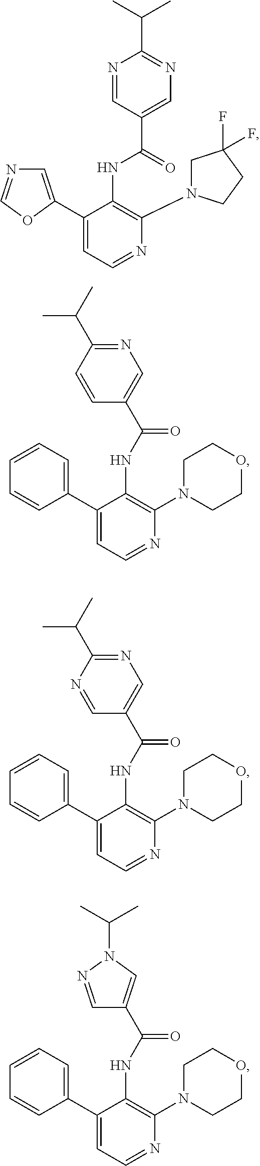 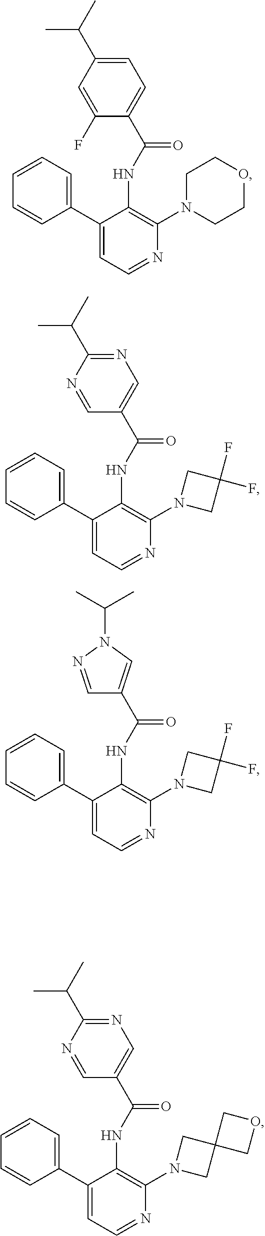 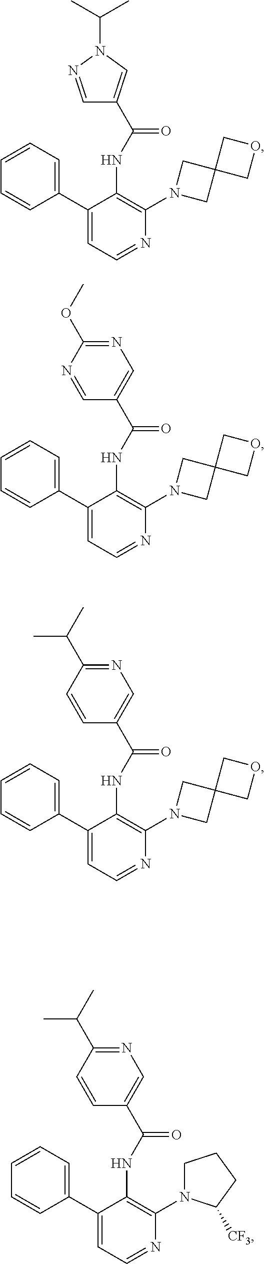 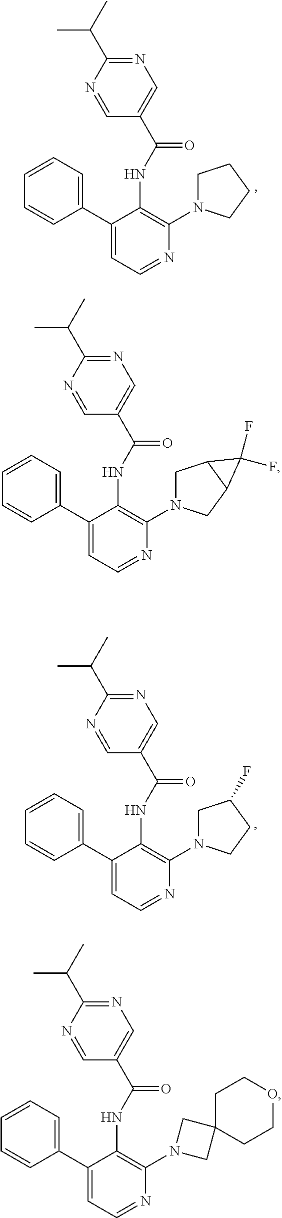 [SMILES: CC(C)c1ncc(C(=O)Nc2c(-c3ccccc3)ccnc2N2CCCC2)cn1 ; CC(C)c1ncc(C(=O)Nc2c(-c3ccccc3)ccnc2N2CC3C(C2)C3(F)F)cn1 ; CC(C)c1ncc(C(=O)Nc2c(-c3ccccc3)ccnc2N2CC[C@@H](F)C2)cn1 ; CC(C)c1ncc(C(=O)Nc2c(-c3ccccc3)ccnc2N2CC3(CCOCC3)C2)cn1] 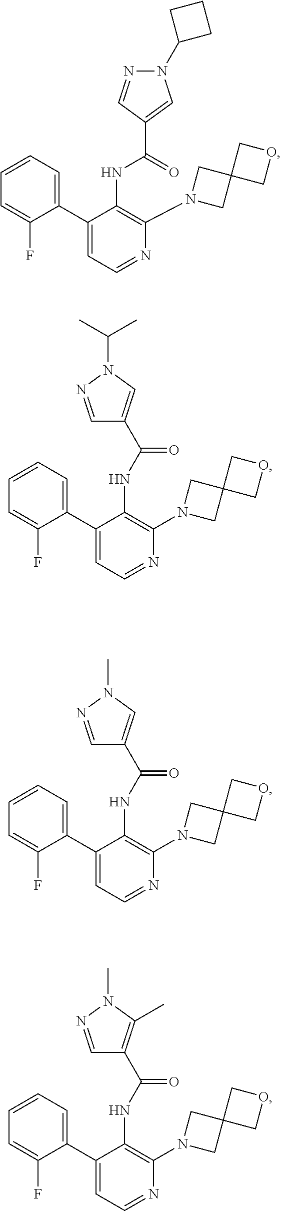 [SMILES: O=C(Nc1c(-c2ccccc2F)ccnc1N1CC2(COC2)C1)c1cnn(C2CCC2)c1 ; CC(C)n1cc(C(=O)Nc2c(-c3ccccc3F)ccnc2N2CC3(COC3)C2)cn1 ; Cn1cc(C(=O)Nc2c(-c3ccccc3F)ccnc2N2CC3(COC3)C2)cn1 ; Cc1c(C(=O)Nc2c(-c3ccccc3F)ccnc2N2CC3(COC3)C2)cnn1C] 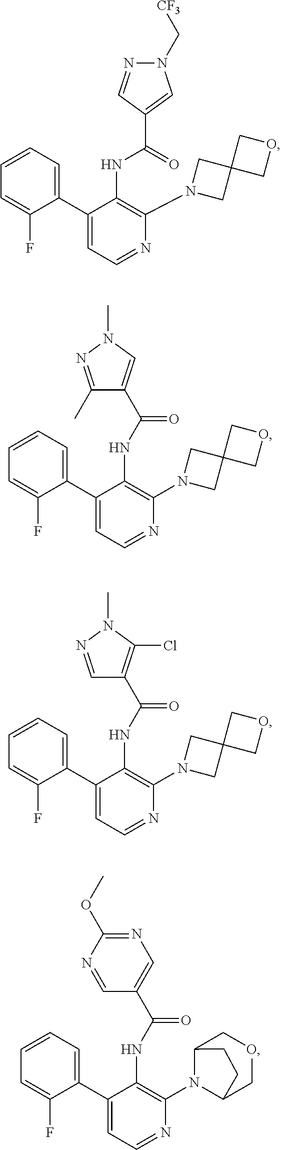 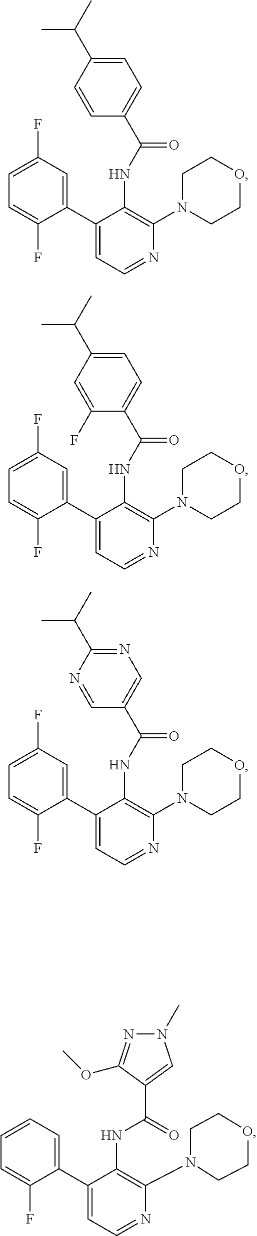 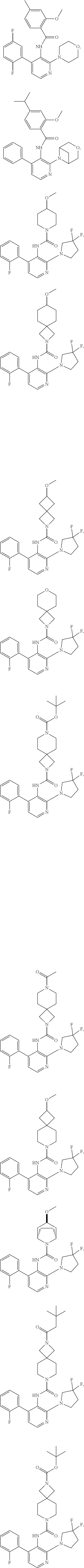 [chemical structure] [chemical structure] [chemical structure] 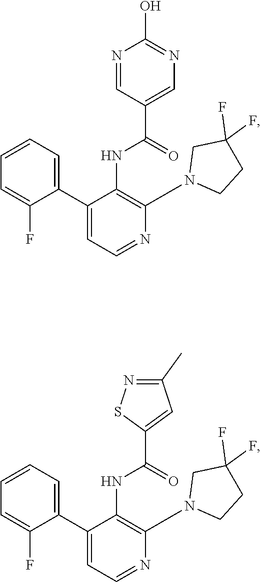 [SMILES: O=C(Nc1c(-c2ccccc2F)ccnc1N1CCC(F)(F)C1)c1cnc(O)nc1 ; Cc1cc(C(=O)Nc2c(-c3ccccc3F)ccnc2N2CCC(F)(F)C2)sn1] 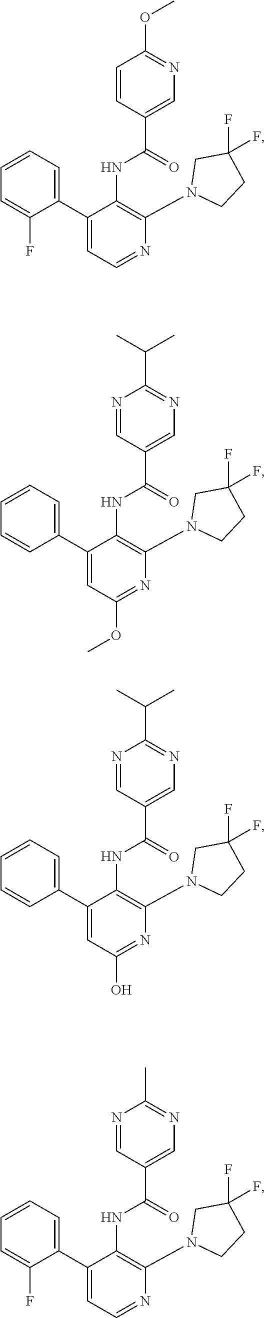 [SMILES: COc1ccc(C(=O)Nc2c(-c3ccccc3F)ccnc2N2CCC(F)(F)C2)cn1 ; COc1cc(-c2ccccc2)c(NC(=O)c2cnc(C(C)C)nc2)c(N2CCC(F)(F)C2)n1 ; CC(C)c1ncc(C(=O)Nc2c(-c3ccccc3)cc(O)nc2N2CCC(F)(F)C2)cn1 ; Cc1ncc(C(=O)Nc2c(-c3ccccc3F)ccnc2N2CCC(F)(F)C2)cn1] 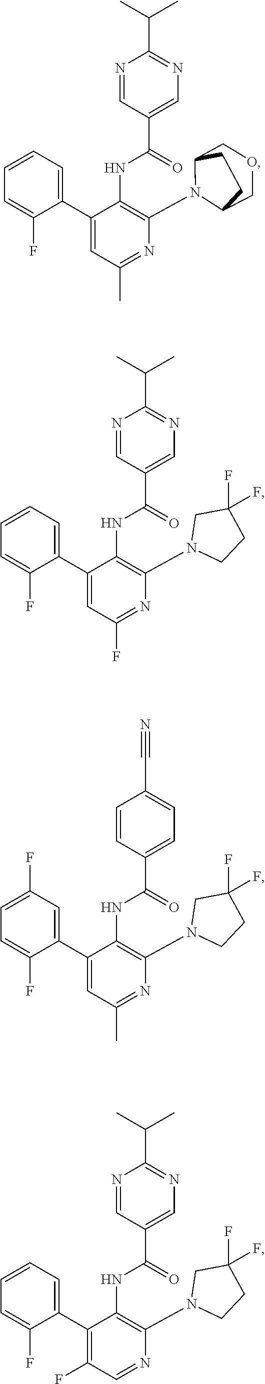 [SMILES: Cc1cc(-c2ccccc2F)c(NC(=O)c2cnc(C(C)C)nc2)c(N2[C@@H]3CC[C@H]2COC3)n1 ; CC(C)c1ncc(C(=O)Nc2c(-c3ccccc3F)cc(F)nc2N2CCC(F)(F)C2)cn1 ; Cc1cc(-c2cc(F)ccc2F)c(NC(=O)c2ccc(C#N)cc2)c(N2CCC(F)(F)C2)n1 ; CC(C)c1ncc(C(=O)Nc2c(N3CCC(F)(F)C3)ncc(F)c2-c2ccccc2F)cn1] 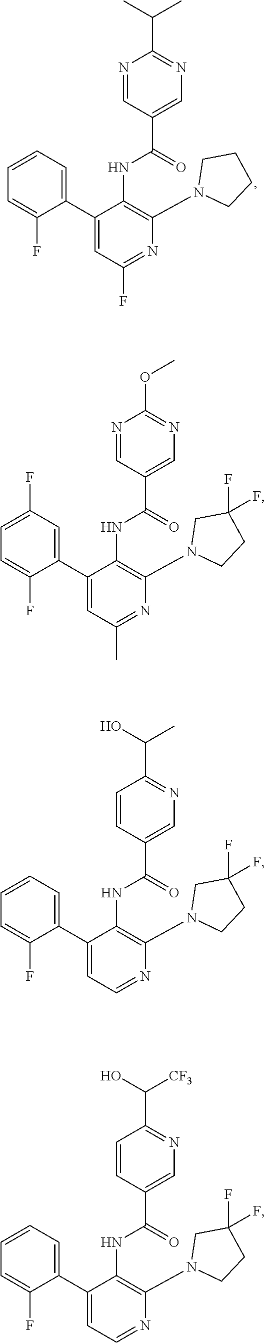 [SMILES: CC(C)c1ncc(C(=O)Nc2c(-c3ccccc3F)cc(F)nc2N2CCCC2)cn1 ; COc1ncc(C(=O)Nc2c(-c3cc(F)ccc3F)cc(C)nc2N2CCC(F)(F)C2)cn1 ; CC(O)c1ccc(C(=O)Nc2c(-c3ccccc3F)ccnc2N2CCC(F)(F)C2)cn1 ; O=C(Nc1c(-c2ccccc2F)ccnc1N1CCC(F)(F)C1)c1ccc(C(O)C(F)(F)F)nc1] 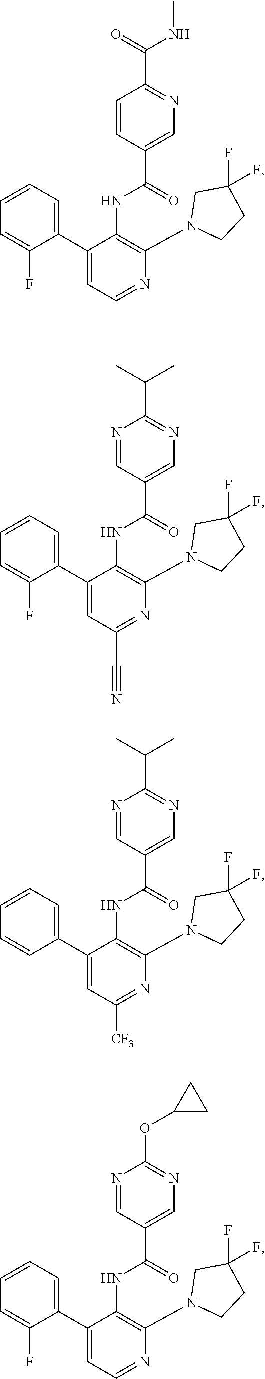 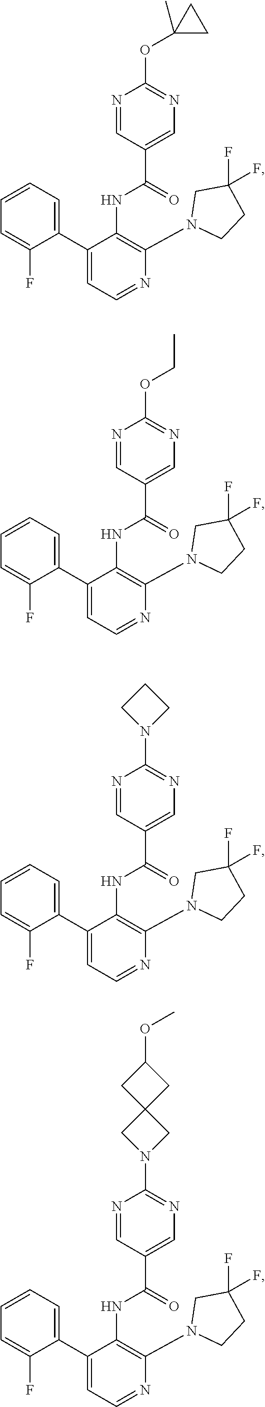 [SMILES: CC1(Oc2ncc(C(=O)Nc3c(-c4ccccc4F)ccnc3N3CCC(F)(F)C3)cn2)CC1 ; CCOc1ncc(C(=O)Nc2c(-c3ccccc3F)ccnc2N2CCC(F)(F)C2)cn1 ; O=C(Nc1c(-c2ccccc2F)ccnc1N1CCC(F)(F)C1)c1cnc(N2CCC2)nc1 ; COC1CC2(C1)CN(c1ncc(C(=O)Nc3c(-c4ccccc4F)ccnc3N3CCC(F)(F)C3)cn1)C2] 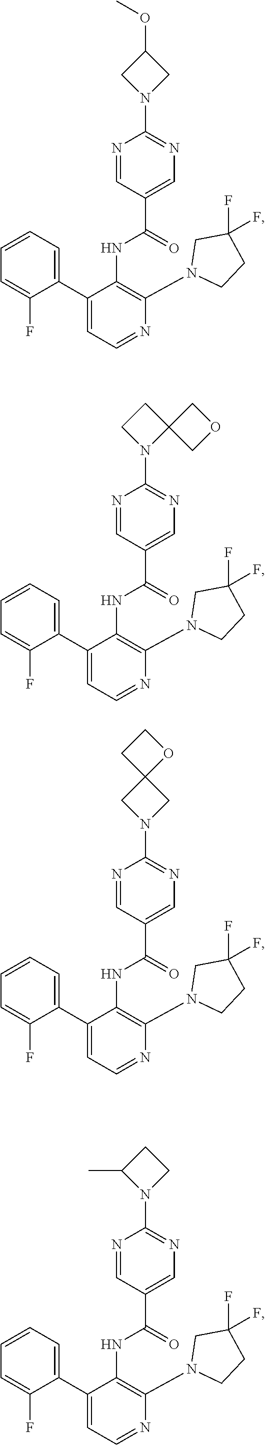 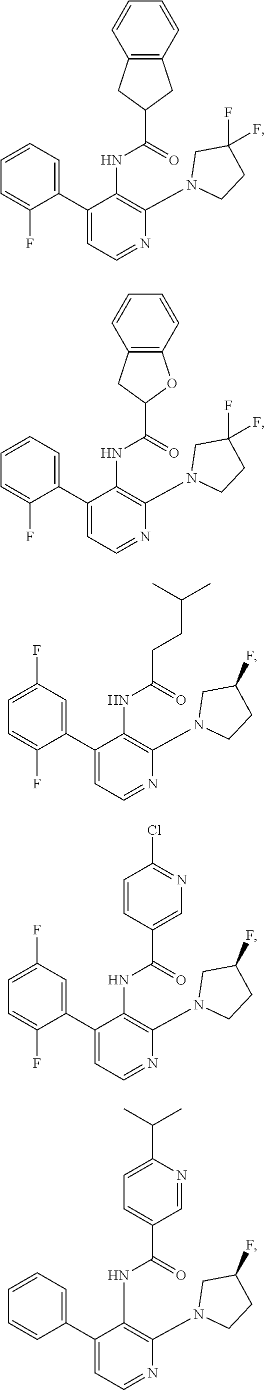 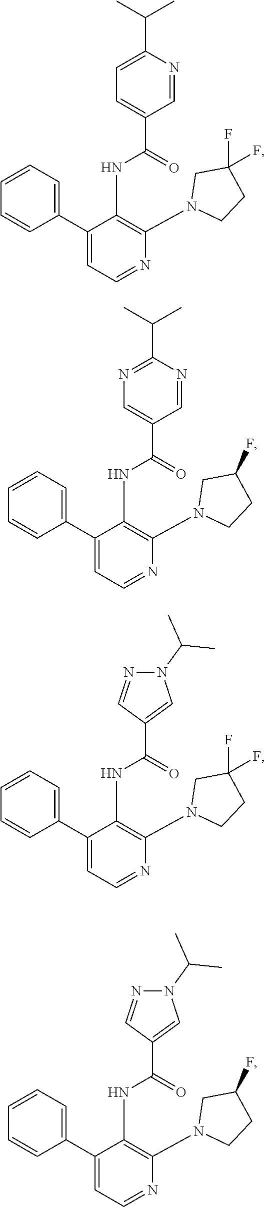 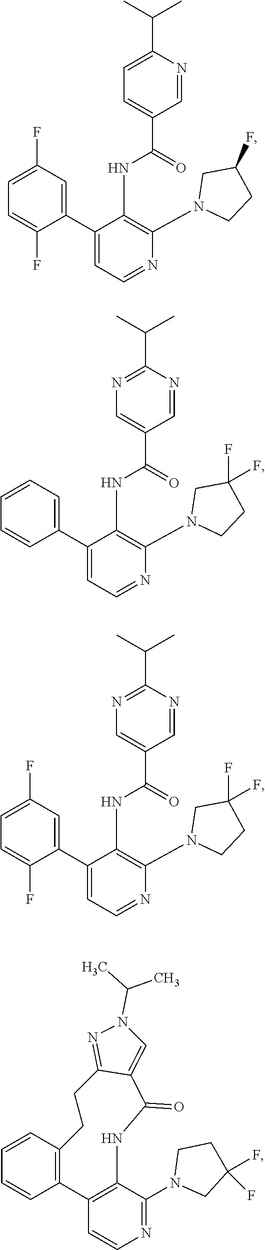 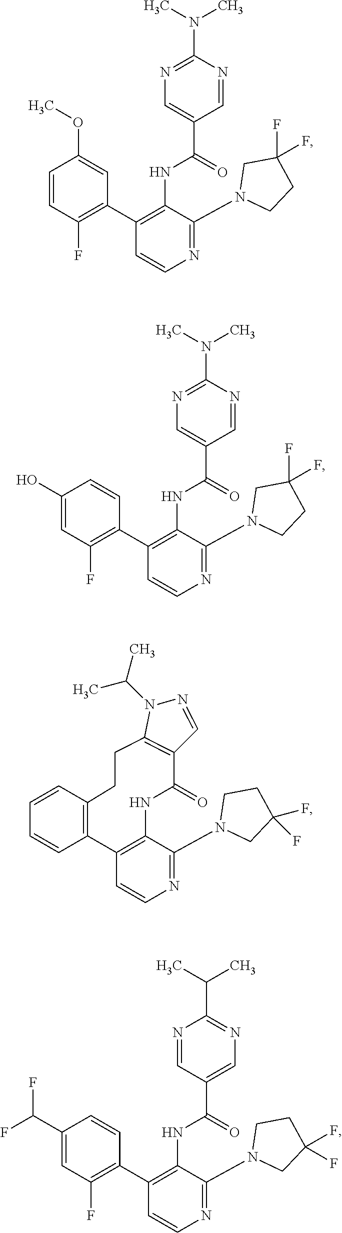 [SMILES: COc1ccc(F)c(-c2ccnc(N3CCC(F)(F)C3)c2NC(=O)c2cnc(N(C)C)nc2)c1 ; CN(C)c1ncc(C(=O)Nc2c(-c3ccc(O)cc3F)ccnc2N2CCC(F)(F)C2)cn1 ; CC(C)n1ncc2c1CCc1ccccc1-c1ccnc(N3CCC(F)(F)C3)c1NC2=O ; CC(C)c1ncc(C(=O)Nc2c(-c3ccc(C(F)F)cc3F)ccnc2N2CCC(F)(F)C2)cn1] 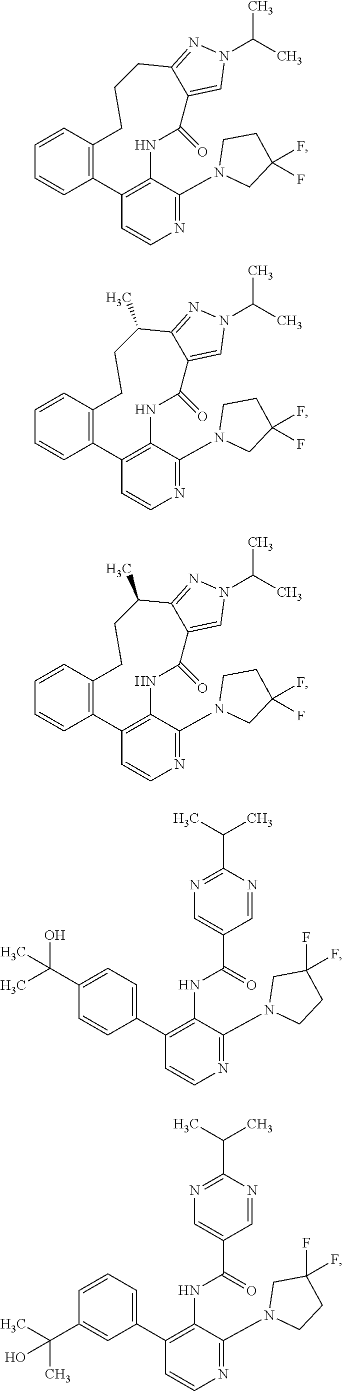 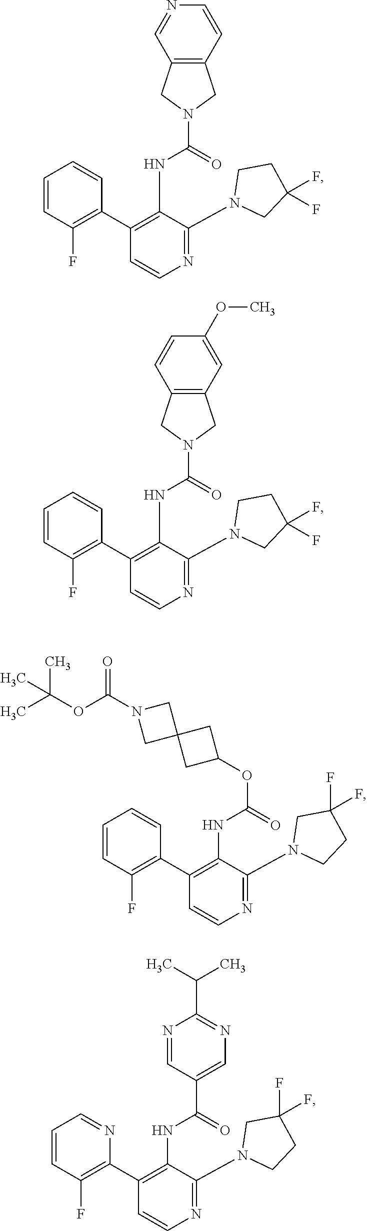 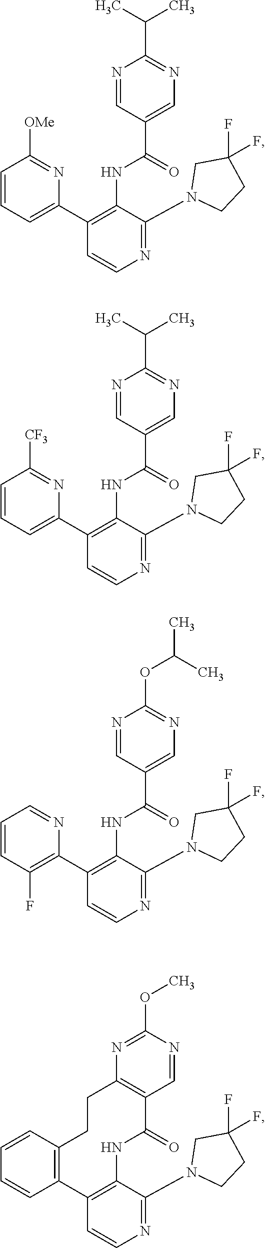 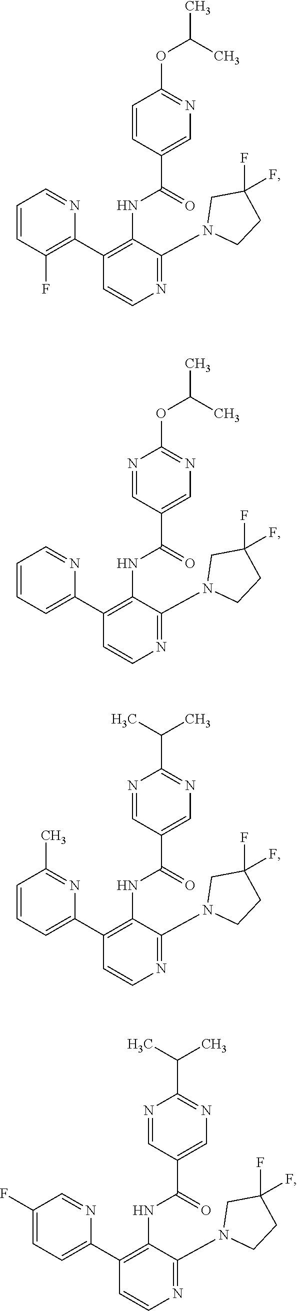 [SMILES: CC(C)Oc1ccc(C(=O)Nc2c(-c3ncccc3F)ccnc2N2CCC(F)(F)C2)cn1 ; CC(C)Oc1ncc(C(=O)Nc2c(-c3ccccn3)ccnc2N2CCC(F)(F)C2)cn1 ; Cc1cccc(-c2ccnc(N3CCC(F)(F)C3)c2NC(=O)c2cnc(C(C)C)nc2)n1 ; CC(C)c1ncc(C(=O)Nc2c(-c3ccc(F)cn3)ccnc2N2CCC(F)(F)C2)cn1] 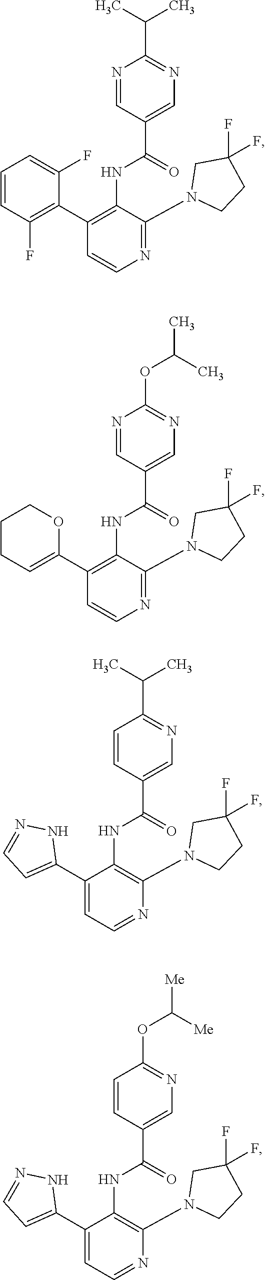 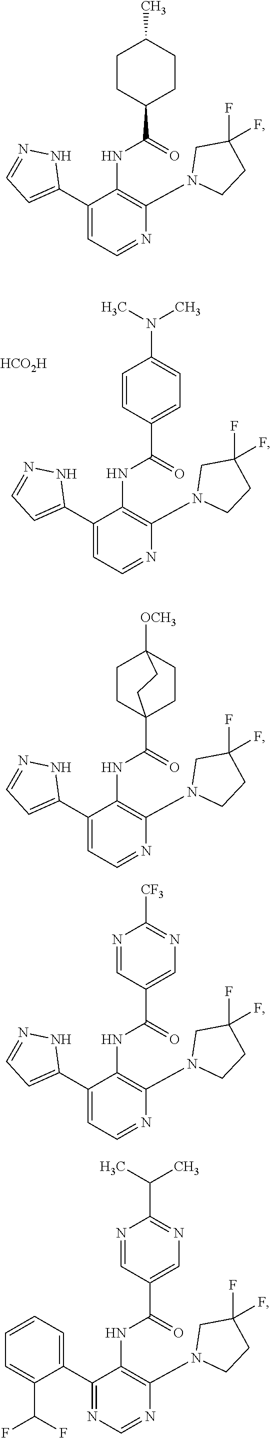 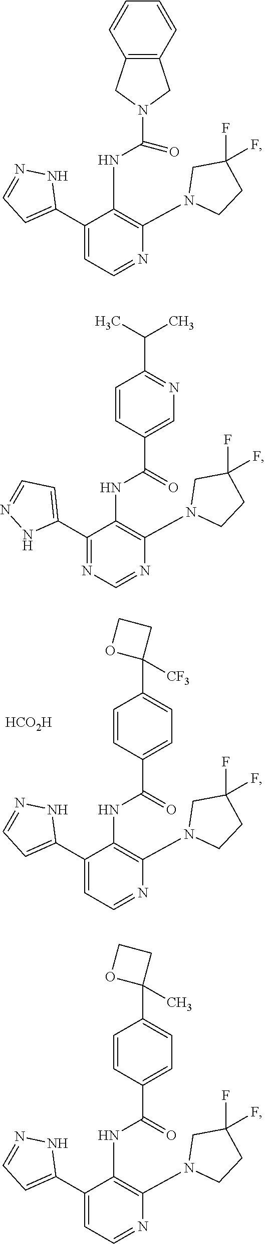 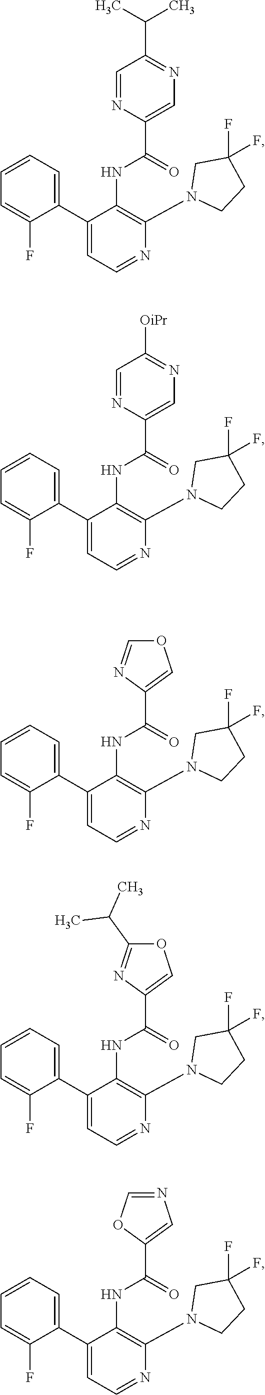 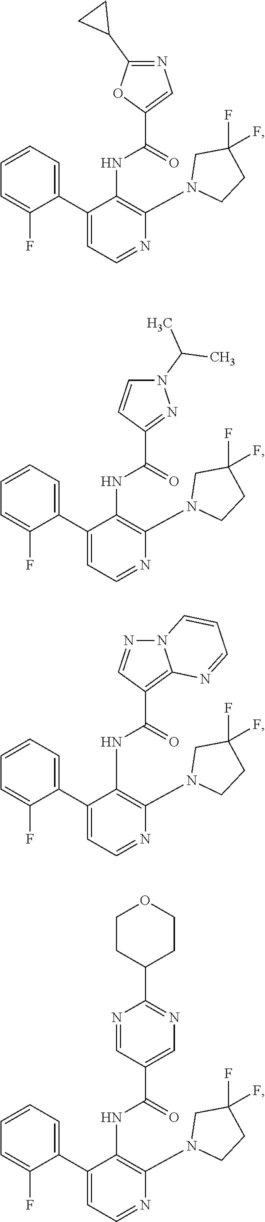 [SMILES: O=C(Nc1c(-c2ccccc2F)ccnc1N1CCC(F)(F)C1)c1cnc(C2CC2)o1 ; CC(C)n1ccc(C(=O)Nc2c(-c3ccccc3F)ccnc2N2CCC(F)(F)C2)n1 ; O=C(Nc1c(-c2ccccc2F)ccnc1N1CCC(F)(F)C1)c1cnn2cccnc12 ; O=C(Nc1c(-c2ccccc2F)ccnc1N1CCC(F)(F)C1)c1cnc(C2CCOCC2)nc1] 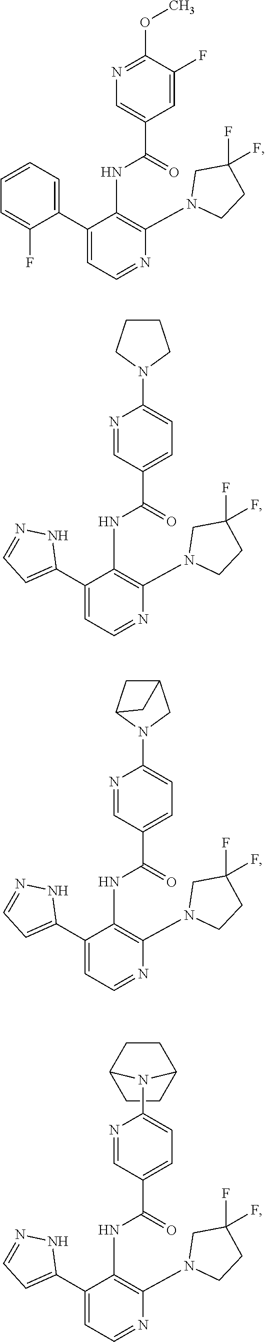 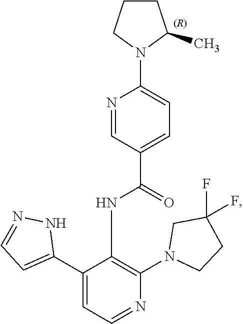 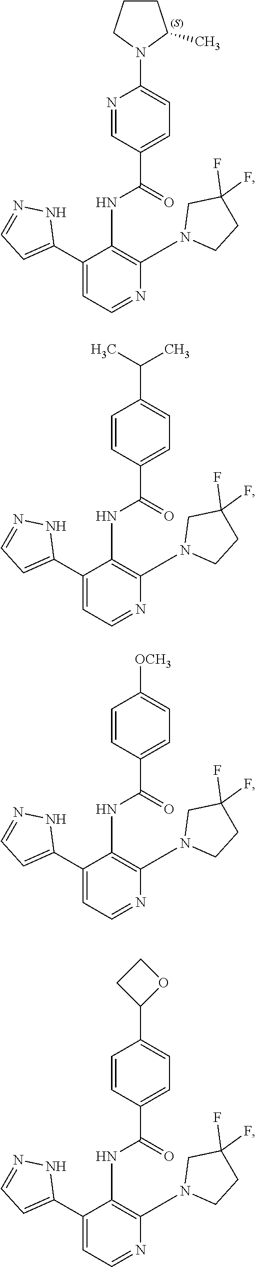 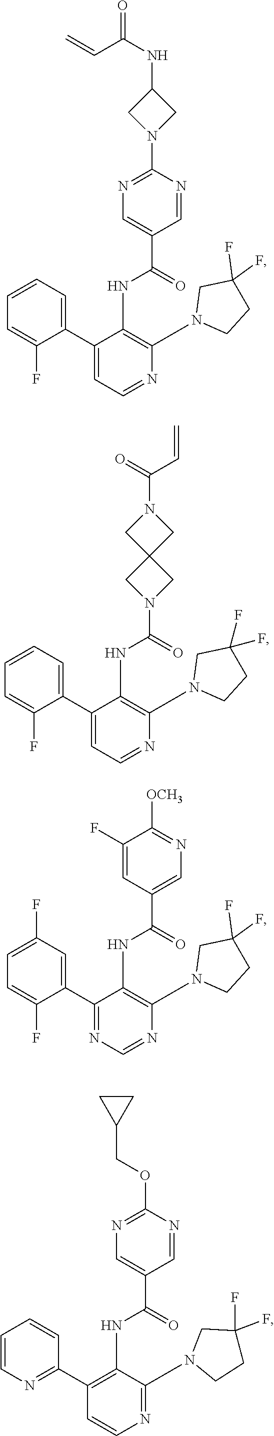 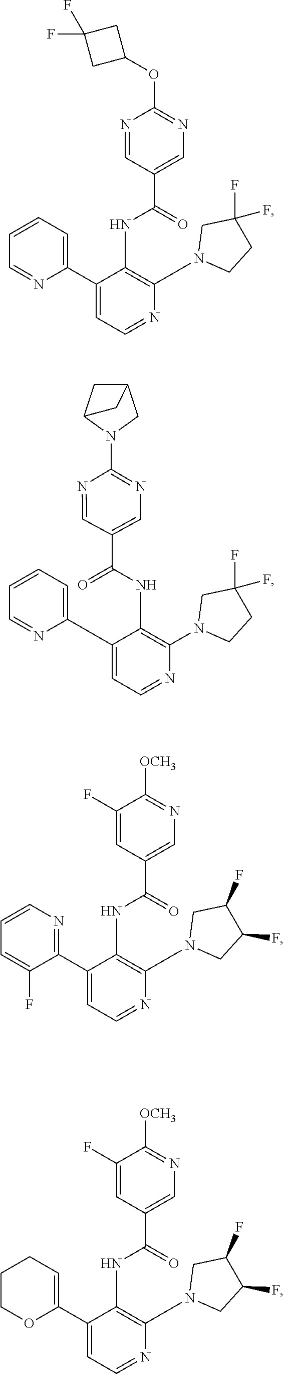 [SMILES: O=C(Nc1c(-c2ccccn2)ccnc1N1CCC(F)(F)C1)c1cnc(OC2CC(F)(F)C2)nc1 ; O=C(Nc1c(-c2ccccn2)ccnc1N1CCC(F)(F)C1)c1cnc(N2CC3CC2C3)nc1 ; COc1ncc(C(=O)Nc2c(-c3ncccc3F)ccnc2N2C[C@@H](F)[C@@H](F)C2)cc1F ; COc1ncc(C(=O)Nc2c(C3=CCCCO3)ccnc2N2C[C@@H](F)[C@@H](F)C2)cc1F] 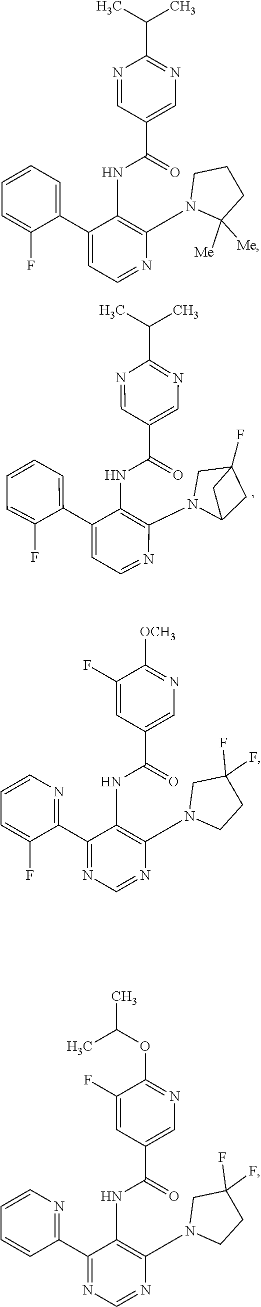 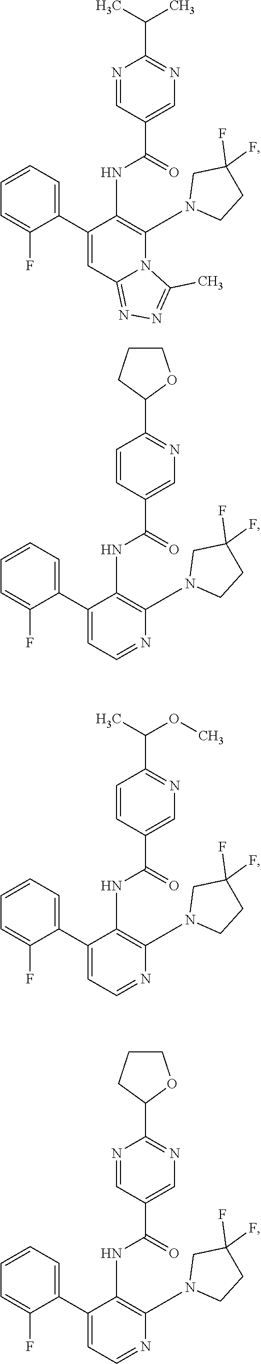 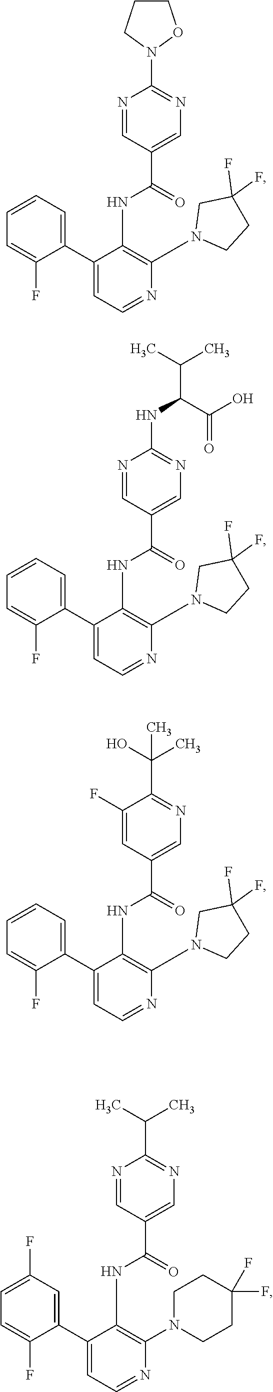 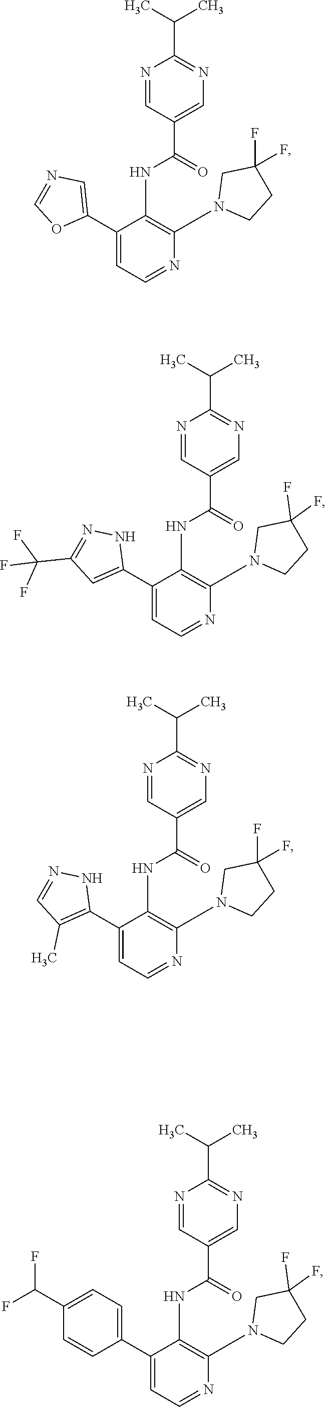 [SMILES: CC(C)c1ncc(C(=O)Nc2c(-c3cnco3)ccnc2N2CCC(F)(F)C2)cn1 ; CC(C)c1ncc(C(=O)Nc2c(-c3cc(C(F)(F)F)n[nH]3)ccnc2N2CCC(F)(F)C2)cn1 ; Cc1cn[nH]c1-c1ccnc(N2CCC(F)(F)C2)c1NC(=O)c1cnc(C(C)C)nc1 ; CC(C)c1ncc(C(=O)Nc2c(-c3ccc(C(F)F)cc3)ccnc2N2CCC(F)(F)C2)cn1] 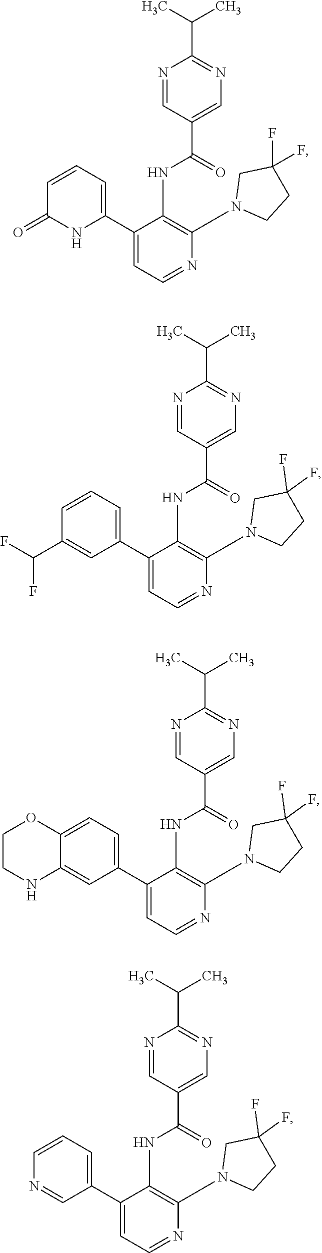 [SMILES: CC(C)c1ncc(C(=O)Nc2c(-c3cccc(=O)[nH]3)ccnc2N2CCC(F)(F)C2)cn1 ; CC(C)c1ncc(C(=O)Nc2c(-c3cccc(C(F)F)c3)ccnc2N2CCC(F)(F)C2)cn1 ; CC(C)c1ncc(C(=O)Nc2c(-c3ccc4c(c3)NCCO4)ccnc2N2CCC(F)(F)C2)cn1 ; CC(C)c1ncc(C(=O)Nc2c(-c3cccnc3)ccnc2N2CCC(F)(F)C2)cn1] 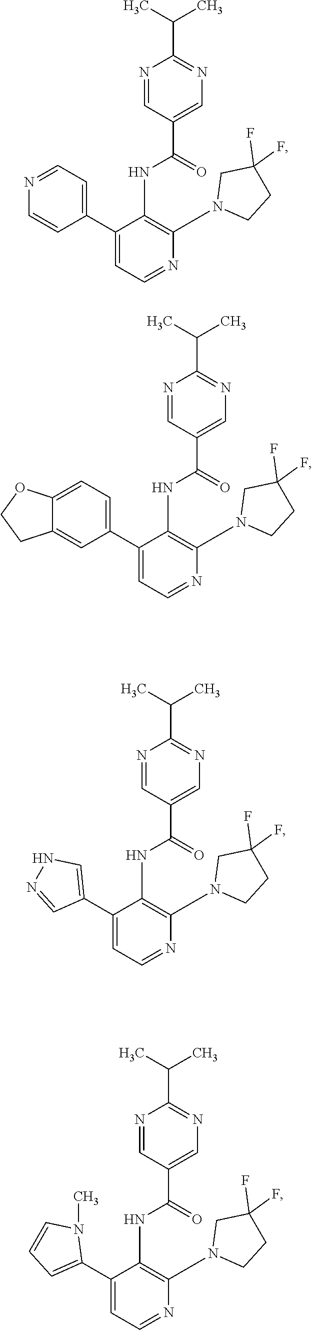 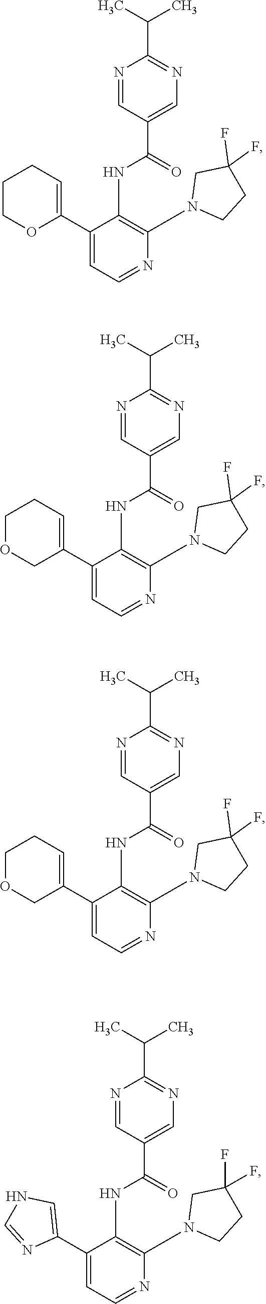 [SMILES: CC(C)c1ncc(C(=O)Nc2c(C3=CCCCO3)ccnc2N2CCC(F)(F)C2)cn1 ; CC(C)c1ncc(C(=O)Nc2c(C3=CCCOC3)ccnc2N2CCC(F)(F)C2)cn1 ; CC(C)c1ncc(C(=O)Nc2c(C3=CCCOC3)ccnc2N2CCC(F)(F)C2)cn1 ; CC(C)c1ncc(C(=O)Nc2c(-c3c[nH]cn3)ccnc2N2CCC(F)(F)C2)cn1] 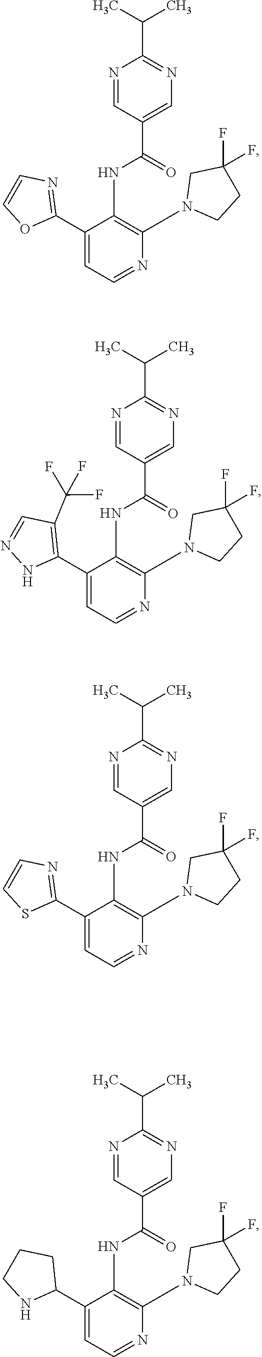 [SMILES: CC(C)c1ncc(C(=O)Nc2c(-c3ncco3)ccnc2N2CCC(F)(F)C2)cn1 ; CC(C)c1ncc(C(=O)Nc2c(-c3[nH]ncc3C(F)(F)F)ccnc2N2CCC(F)(F)C2)cn1 ; CC(C)c1ncc(C(=O)Nc2c(-c3nccs3)ccnc2N2CCC(F)(F)C2)cn1 ; CC(C)c1ncc(C(=O)Nc2c(C3CCCN3)ccnc2N2CCC(F)(F)C2)cn1] 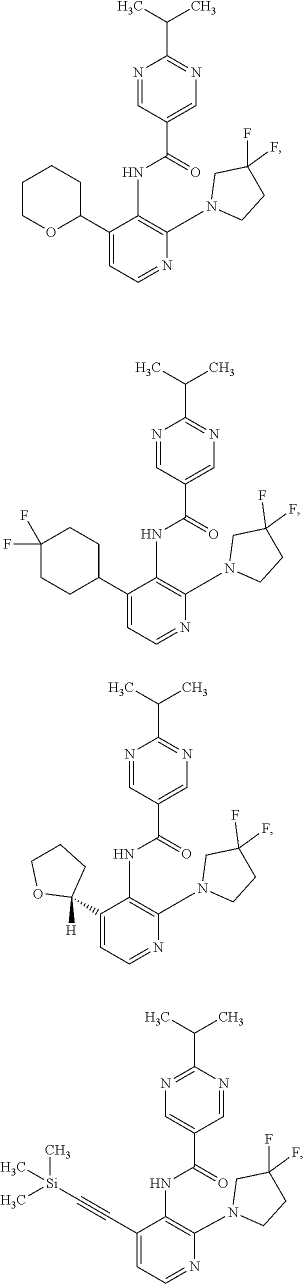 [SMILES: CC(C)c1ncc(C(=O)Nc2c(C3CCCCO3)ccnc2N2CCC(F)(F)C2)cn1 ; CC(C)c1ncc(C(=O)Nc2c(C3CCC(F)(F)CC3)ccnc2N2CCC(F)(F)C2)cn1 ; CC(C)c1ncc(C(=O)Nc2c([C@H]3CCCO3)ccnc2N2CCC(F)(F)C2)cn1 ; CC(C)c1ncc(C(=O)Nc2c(C#C[Si](C)(C)C)ccnc2N2CCC(F)(F)C2)cn1] 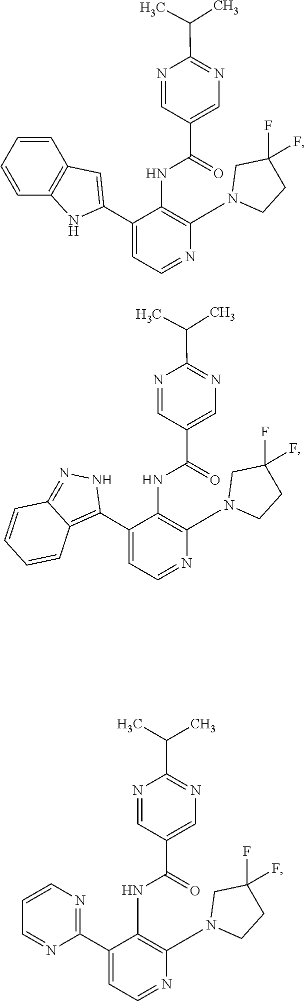 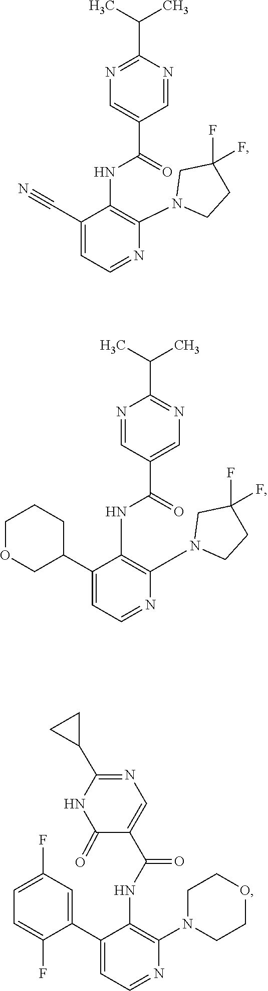 [SMILES: CC(C)c1ncc(C(=O)Nc2c(C#N)ccnc2N2CCC(F)(F)C2)cn1 ; CC(C)c1ncc(C(=O)Nc2c(C3CCCOC3)ccnc2N2CCC(F)(F)C2)cn1 ; O=C(Nc1c(-c2cc(F)ccc2F)ccnc1N1CCOCC1)c1cnc(C2CC2)[nH]c1=O] 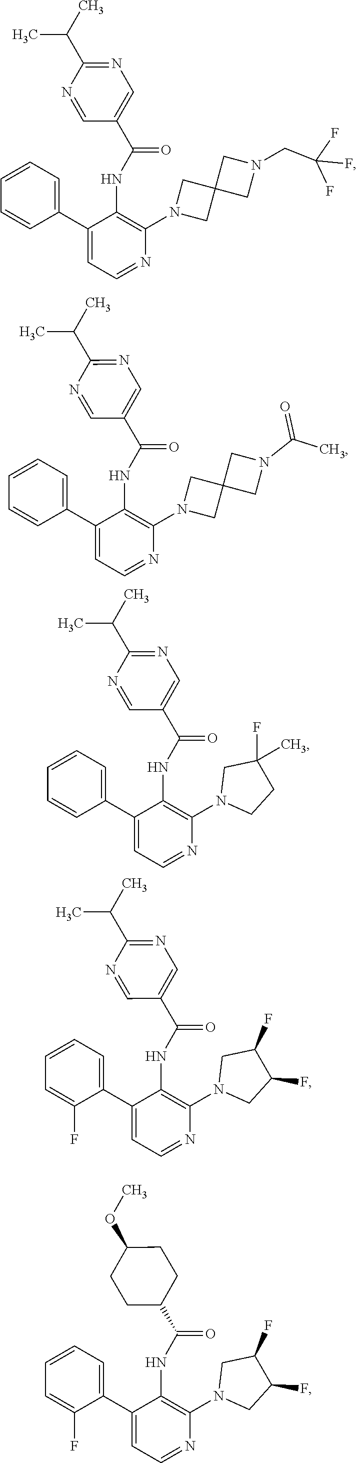 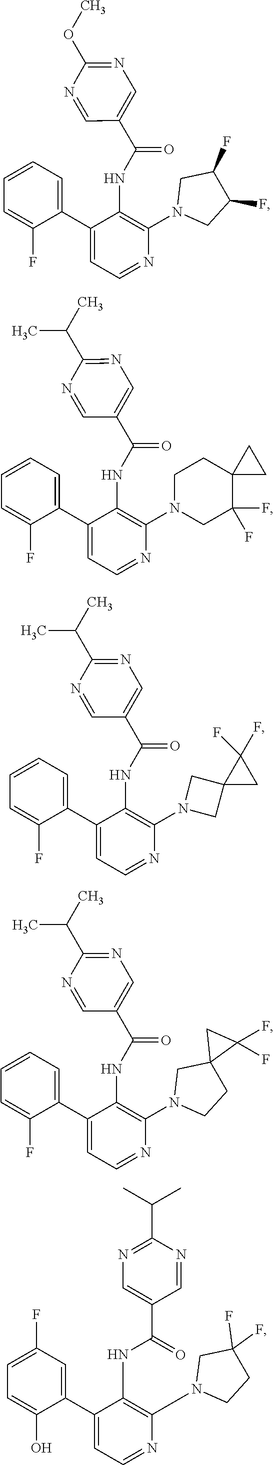 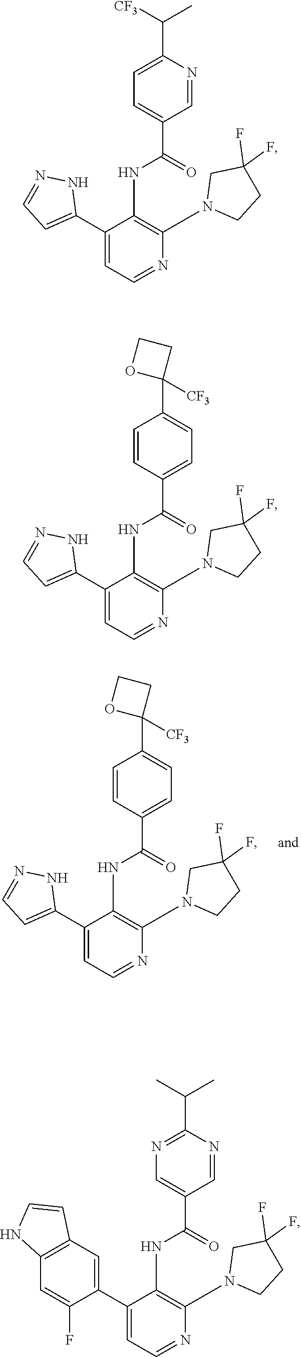
as a stereoisomer, enantiomer, or tautomer thereof or a mixture thereof; or a pharmaceutically acceptable salt, solvate, or prodrug thereof.

74. A pharmaceutical composition comprising a pharmaceutically acceptable excipient and a compound of formula (I): 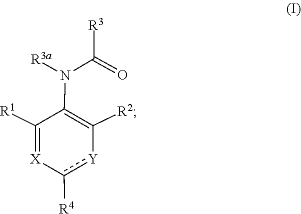
wherein [symbol]
represents a double or single bond such that all valences are satisfied;
Y is N or NR^{4a};
X is C(R⁷) or N;
R¹ is selected from: 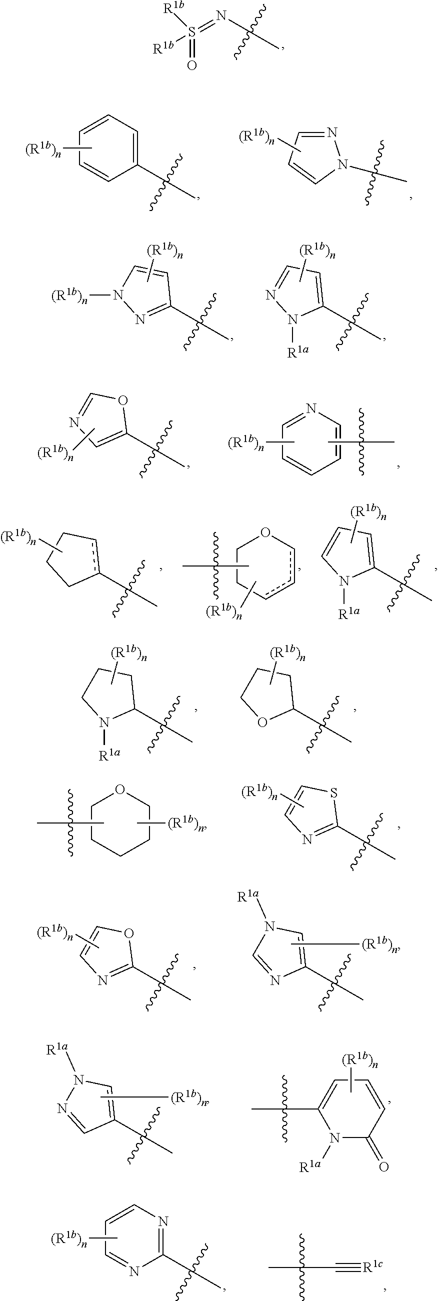
wherein:
each occurrence of [symbol] independently represents a double or single bond such that all valences are satisfied;
n is 0, 1, 2, 3, 4, or 5;
R^{1a} is hydrogen, or alkyl;
each Rib is independently halo, alkyl, haloalkyl, cyano, heterocyclylalkyl,
—R⁸—N(R⁹)₂, —R⁸—C(═O)N(R⁹)₂, or —R⁸—OR⁹
or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
R^{1c} is N or —Si(CH₃)₃;
R² is selected from: 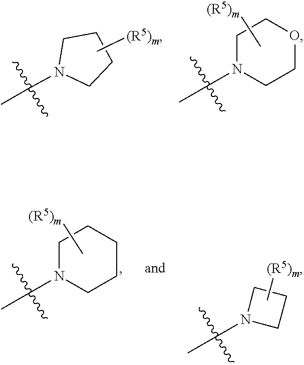
wherein:
m is 0, 1, 2, 3, or 4;
each R⁵ is independently halo, alkyl, haloalkyl or —R¹⁰—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain;
R³ is alkyl, —R⁸—N(R⁹)₂, —R⁸—OR⁹, or
R³ is selected from: 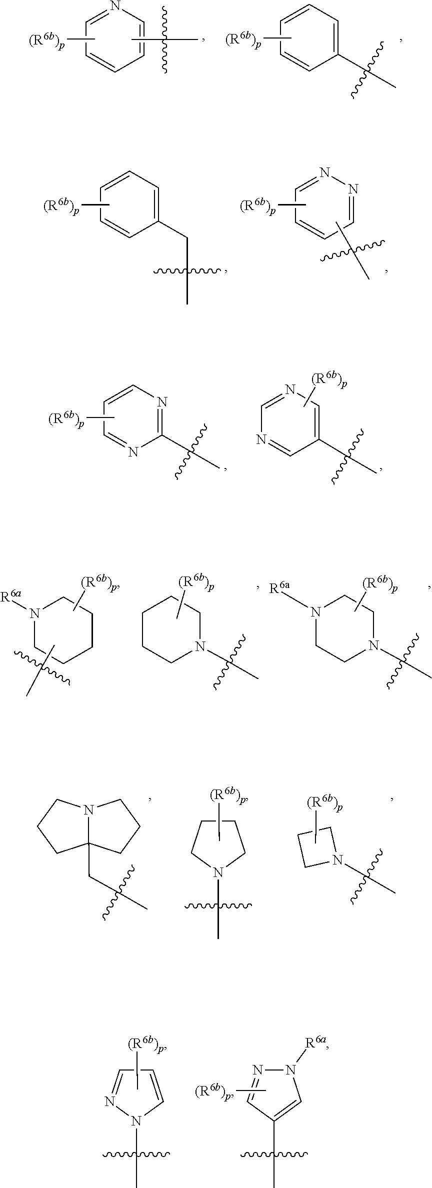 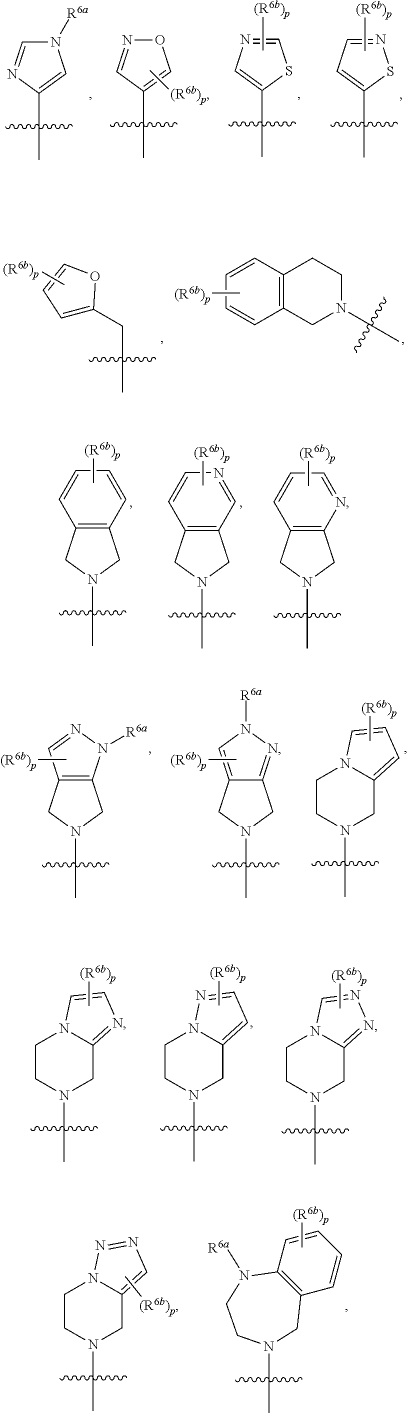 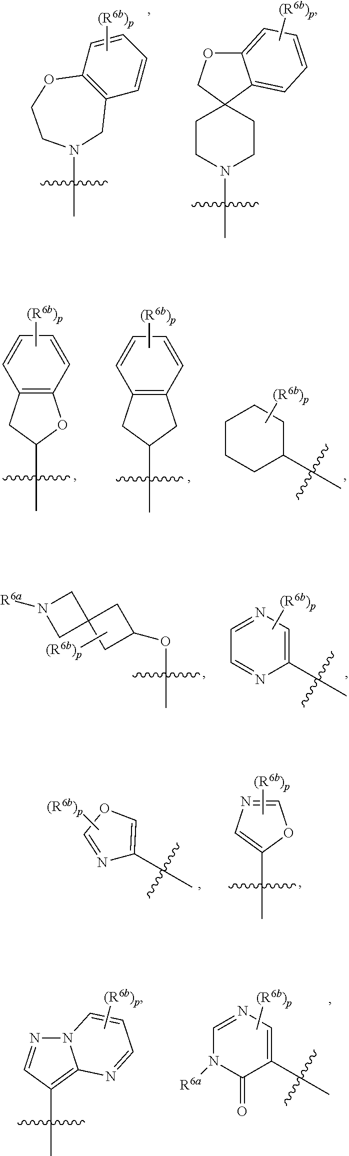
wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain;
or an occurrence of R^{6b} and an occurrence of Rib join to form an optionally substituted alkylene chain;
R^{3a} is hydrogen or alkyl;
R⁴ is hydrogen, alkyl, —R⁸—OR⁹, halo, haloalkyl, or cyano;
or R⁴ together with the carbon to which it is attached, joins with R^{4a} together with the nitrogen to which it is attached to form an optionally substituted 5-membered N-heteroaryl;
R⁷ is hydrogen, alkyl, halo, or —R⁸—OR⁹ each R⁸ is independently a direct bond or an optionally substituted alkylene chain;
each R⁹ is independently hydrogen, alkyl, haloalkyl, carboxyalkyl, optionally substituted cycloalkyl, optionally substituted cycloalkylalkyl, or optionally substituted aryl; and
or two R⁹'s, together with the nitrogen to which they are both attached, form an optionally substituted heterocyclyl;
provided that:
when X is N, R³ is selected from: 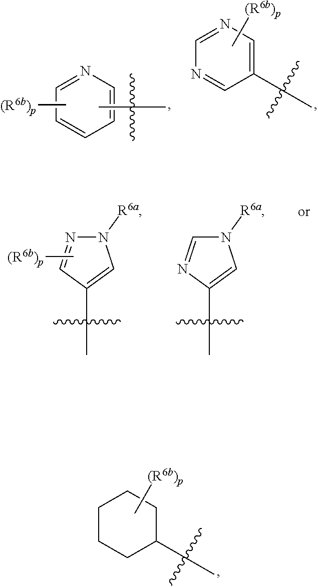
as a stereoisomer, enantiomer, or tautomer thereof or a mixture thereof, or a pharmaceutically acceptable salt, solvate, or prodrug thereof.

75. A method of treating a disease or condition in a mammal modulated by a voltage-gated sodium channel, wherein the method comprises administering to a mammal in need thereof a therapeutically effective amount of a compound of formula (I): 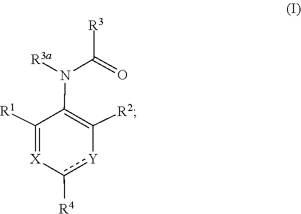
wherein: [symbol] [symbol]
represents a double or single bond such that all valences are satisfied;
Y is N or NR^{4a};
X is C(R⁷) or N;
R¹ is selected from: 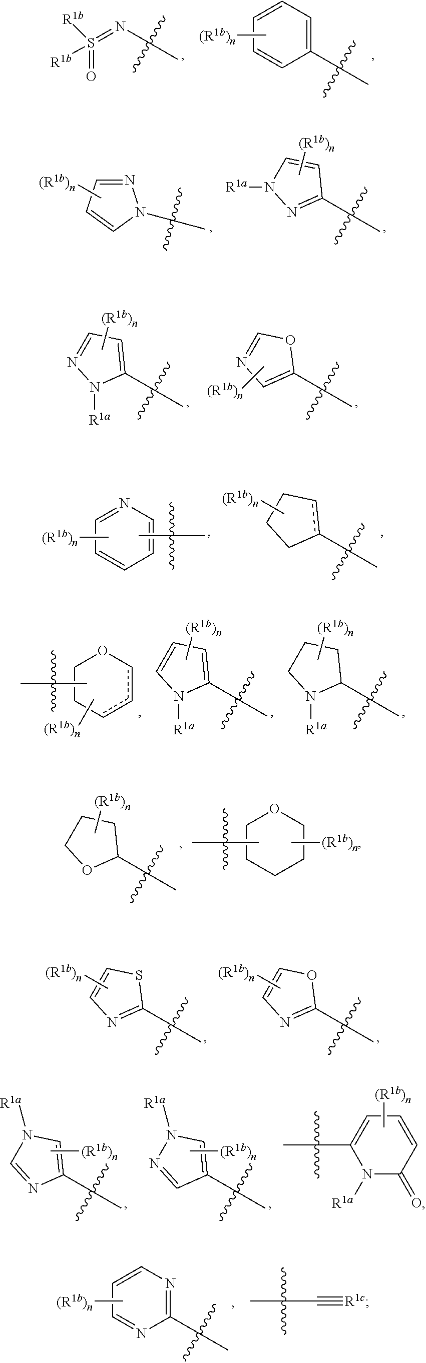
wherein:
each occurrence of independently represents a double or single bond such that all valences are satisfied; [symbol] n is 0, 1, 2, 3, 4, or 5;
R^{1a} is hydrogen, or alkyl;
each R^{1b} is independently halo, alkyl, haloalkyl, cyano, heterocyclylalkyl,
—R⁸—N(R⁹)₂, —R⁸—C(═O)N(R⁹)₂, or —R⁸—OR⁹ or two R^{1b}'s attached to adjacent carbons, together with the carbons to which they are attached, form an optionally substituted N-heteroaryl, an optionally substituted N-heterocycylyl, an optionally substituted O-heterocycylyl, or an optionally substituted aryl;
R^{1c} is N or —Si(CH₃)₃;
R² is selected from: 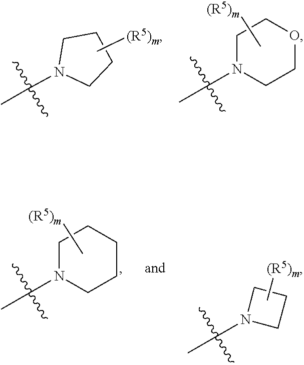
wherein:
m is 0, 1, 2, 3, or 4;
each R⁵ is independently halo, alkyl, haloalkyl or —Rᵢ°—CN;
or two R⁵'s, together with the carbon to which they are both attached, form an an optionally substituted O-heterocyclyl;
or two R⁵'s, together with the carbon to which they are both attached, form an optionally substituted N-heterocyclyl;
or two R⁵'s, together with the carbon to which they are both attached, form an optionally substituted cycloalkyl; and
or two R⁵'s join to form an optionally substituted alkylene chain;
R³ is alkyl, —R⁸—N(R⁹)₂, —R⁸—OR⁹, or
R³ is selected from: 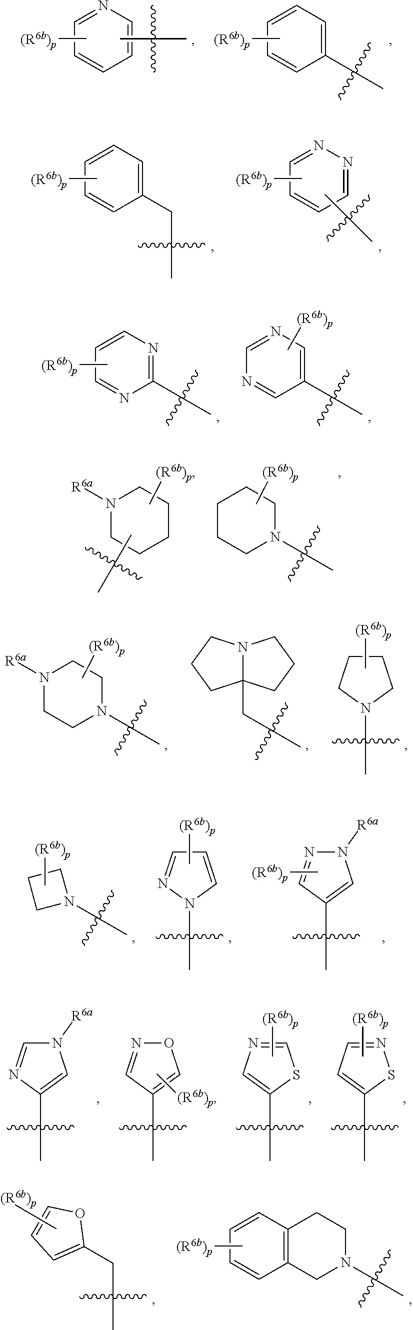 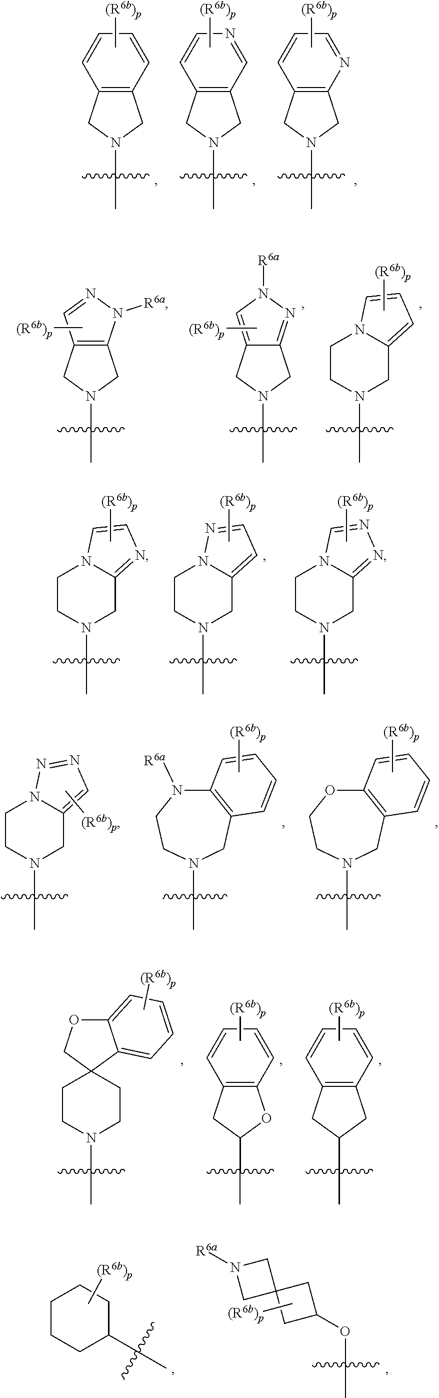 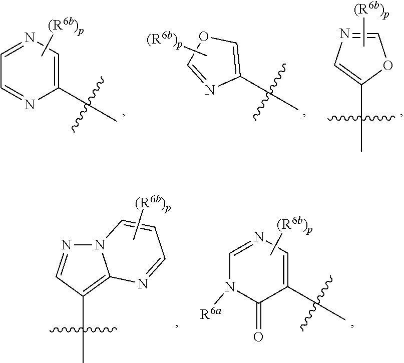
wherein:
p is 0, 1, 2, 3, 4, or 5;
R^{6a} is hydrogen, alkyl, cycloalkyl, haloalkyl, —C(═O)R⁹, optionally substituted arylalkyl, or optionally substituted heteroaryl;
each R^{6b} is independently alkyl, halo, haloalkyl, —R⁸—OR⁹, —R⁸—N(R⁹)₂, —R⁸—C(═O)OR⁹, —R⁸—C(═O)N(R⁹)₂,optionally substituted cycloalkyl, optionally substituted aryl, optionally substituted heterocyclyl, or optionally substituted heteroaryl;
or two R^{6b}'s, together with the carbon to which they are both attached, form an optionally substituted N-heterocyclyl;
or two R^{6b}'s, together with the carbon to which they are both attached, form an optionally substituted O-heterocyclyl;
or two R^{6b}'s, together with the carbon to which they are both attached, form an optionally substituted cycloalkyl;
or two R^{6b}'s join to form an optionally substituted alkylene chain;
or an occurrence of R^{6b} and an occurrence of R^{1b} join to form an optionally substituted alkylene chain;
R^{3a} is hydrogen or alkyl;
R⁴ is hydrogen, alkyl, —R⁸—OR⁹, halo, haloalkyl, or cyano;
or R⁴ together with the carbon to which it is attached, joins with R^{4a} together with the nitrogen to which it is attached to form an optionally substituted 5-membered N-heteroaryl;
R⁷ is hydrogen, alkyl, halo, or —R⁸—OR⁹ each R⁸ is independently a direct bond or an optionally substituted alkylene chain;
each R⁹ is independently hydrogen, alkyl, haloalkyl, carboxyalkyl, optionally substituted cycloalkyl, optionally substituted cycloalkylalkyl, or optionally substituted aryl; and
or two R⁹'s, together with the nitrogen to which they are both attached, form an optionally substituted heterocyclyl;
provided that:
when X is N, R³ is selected from: 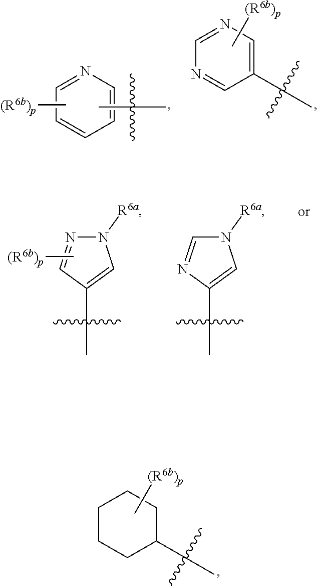
as a stereoisomer, enantiomer, or tautomer thereof or a mixture thereof, or a pharmaceutically acceptable salt, solvate, or prodrug thereof.

76. The method of claim 75, wherein the disease or condition is selected from epilepsy, seizure disorders, partial seizures, generalized seizures, photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures plus (GEFS+), Rett syndrome, multiple sclerosis, Schizophrenia, autism, ataxia, hypotonia and paroxysmal dyskinesia, Alzheimer's disease, Tauopathies, Pick's disease, progressive supranuclear palsy, corticobasal syndrome, frontotemporal dementias, Argyrophilic grain disease, frontotemporal lobar degeneration, globular glial tauopathies, MAPT mutation, primary age-related tauopathy, neurofibrillary tangle dementia, chronic traumatic encephalopathy (CTE), aging-related tau astrogliopathy, Richardson syndrome, Down Syndrome, parkinsonism, pure akinesia with gait freezing, motor neuron symptoms or cerebellar ataxia, posttraumatic stress disorders (PTSD), and any combination thereof.
